(19) United States
(12) Patent Application Publication
(10) Pub. No.: US 2014/0141488 A1
(43) Pub. Date: May 22, 2014

(54) SEQUENCE OF 55 NEW FOUND PROTEINS AND THEIR APPLICATION

(71) Applicant: Kieu Hoang, Agoura Hills, CA (US)

(72) Inventor: Kieu Hoang, Agoura Hills, CA (US)

(21) Appl. No.: 13/756,034

(22) Filed: Jan. 31, 2013

Related U.S. Application Data
(60) Provisional application No. 61/593,164, filed on Jan. 31, 2012.
(60) Provisional application No. 61/593,183, filed on Jan. 31, 2012.
(60) Provisional application No. 61/593,196, filed on Jan. 31, 2012.
(60) Provisional application No. 61/648,281, filed on May 17, 2012.
(60) Provisional application No. 61/692,273, filed on Aug. 23, 2012.
(60) Provisional application No. 61/710,930, filed on Oct. 8, 2012.

(51) Int. Cl. C07K 14/76; C07K 14/75; C07K 14/745

(52) U.S. Cl. CPC C07K 14/76; C07K 14/7455; C07K 14/75
USPC 435/194; 530/350; 530/381; 530/382; 530/387.1; 530/362; 530/383; 530/395

(57) ABSTRACT

Sequence of 55 New Found Proteins—2 new proteins in Cryoprecipitate—8 new proteins in Fraction III—8 new proteins in Prothrombin Complex Concentrate—2 new found proteins in AFCC (Fraction 33)—3 new proteins in Fraction IV and 4 new found proteins in AFOD (Fraction 42)—2 in HemoRAAS®, 3 in FibroRAAS®, 5 in GammaRAAS®, 3 in AFCC®, 1 in Fraction 3-2, 2 in Fraction 3, 4 in FibingluRAAS® (Thrombin), 3 in AFOD®, 1 in AlbuRAAS®, 1 in FibingluRAAS® (High concentrate Fibrinogen), 1 in AFCC® (From fraction IV), 2 in Transferrin from Human Plasma and their name KH1 through KH55, and 16 existing proteins in which good KH healthy cells exists and their application.

### CROSS REFERENCE TO RELATED APPLICATIONS

The benefit under 35 USC 120 is hereby claimed of the filing dates of provisional application No. 61/593,164, filed on Jan. 31, 2012, provisional application No. 61/593,183, filed on Jan. 31, 2012, provisional application No. 61/593,196, filed on Jan. 31, 2012, provisional application No. 61/648,281, filed on May 17, 2012, provisional application No. 61/692,273, filed on Aug. 23, 2012 and provisional application No. 61/710,930, filed on Oct. 8, 2012, all of which are hereby incorporated herein by reference in their entireties.

### BACKGROUND

As we know there are 100,000 proteins in a human being, and each of the protein has its own function to interact with each other or one another to provide all necessary elements to keep the human being healthy. Proteins are known to treat certain diseases, to provide coagulant factors and immune deficiency.

### INVENTION

In order to discover more proteins to cure many diseases that are not curable at the moment. The inventor has discovered 52 newfound proteins:
1) 2 new proteins in Cryoprecipitate
2) 8 new proteins in Fraction III
3) 8 new proteins in Prothrombin Complex Concentrate
4) 2 new found proteins in AFCC
5) 3 new proteins in Fraction IV
6) 4 new found proteins in AFOD
7) 2 new proteins in HemoRAAS® (Human Factor VIII)
8) 3 new proteins in FibroRAAS® (Human Fibrinogen)
9) 5 new proteins in GammaRAAS® (Human Immunoglobulin)
10) 3 new found proteins in AFCC®
11) 1 new proteins in Fraction 3-2
12) 2 new found proteins in Fraction 3
13) 4 new found proteins in FibingluRAAS® Human Thrombin
14) 3 new found protein in AFOD®
15) 1 new found protein in AlbuRAAS® (Human Albumin)
16) 1 new found protein in FibingluRAAS® High Concentrate Human Fibrinogen
Originally we have found only 21 new proteins however when we go into further process to segregate each group of protein in the Fraction 33 from fraction III (AFCC) we have found 2 more new proteins. And the same thing happened with the fraction 42 from fraction IV (AFOD) we have found an additional 4 new proteins. More new proteins may be found when we go into the further process to segregate proteins from fraction 31, 32 or 41, 43 (under different patent application) for example. In fact, we found an additional 26 new proteins. Of this 26 we have been able to identify the sequence for 25 of them. One additional protein found in Human Immunoglobulin is still under investigation for its sequence. The function of which is still under investigation. More new proteins may be found when we go into the further process to segregate proteins from different fractions (under different patent application) for example.
   1) 2 new proteins which have been discovered in cryoprecipitate have been named KH1 and KH2. The function of which are under investigation. See FIG. 7A—Protein Sequence of Cryoprecipitate
   2) 8 new found proteins which have been discovered in Fraction III named KH3, KH4, KH5, KH6, KH7, KH8, KH9 and KH10. The function of which are under investigation. See FIG. 8A—Fraction III. See also FIG. 8B—Fraction III.
   3) 8 new proteins which have been discovered in Prothrombin Complex Concentrate named KH11, KH12, KH13, KH14, KH15, KH16, KH17 and KH18. The function of these proteins are being investigated. See FIG. 9A—Prothrombin Complex Concentrate. See FIG. 9B—Prothrombin Complex Concentrate
   4) 2 new proteins which have been discovered in AFCC (Fraction 33 under different patent application for the AFCC process) named KH19 and KH20. The function of these proteins are being investigated. See FIG. 10A—AFCC. See FIG. 10B—AFCC See FIG. 10C—AFCC
   5) 3 new proteins which have been discovered in Fraction IV named KH21, KH22 and KH23. The function of these proteins are being investigated. FIG. 11—Fraction IV
   6) 4 new proteins which have been discovered in AFOD (Fraction 42 under different patent application for the AFOD process) named KH24, KH25, KH26 and KH27. The function of these proteins are being investigated. See FIG. 12A—AFOD. See FIG. 12 B—AFOD. See FIG. 12C—AFOD

### BRIEF DESCRIPTION OF THE DRAWINGS

FIG. 1—ATIII

FIG. 2—CP98

FIG. 3—TF

FIG. 4 Alpha 1 AT

FIG. 5 FR IV HA (after pasteurization)

FIG. 6—AFCC

FIG. 7A—Protein Sequence of Cryoprecipitate

FIG. 7B—Protein Sequence of Cryoprecipitate

FIG. 8A—Fraction III

FIG. 8B—Fraction III

FIG. 9A—Prothrombin Complex Concentrate

FIG. 9B—Prothrombin Complex Concentrate

FIG. 10A—AFCC

FIG. 10B—AFCC

FIG. 10C—AFCC

FIG. 11—Fraction IV

FIG. 12A—AFOD

FIG. 12B—AFOD

FIG. 12C—AFOD

FIG. 13A—2D Electrophoresis of Anti Thrombin III

FIG. 13B—2D Electrophoresis of CP98

FIG. 13C—2D Electrophoresis of Transferrin

FIG. 13D—2D Electrophoresis of α1-Anti Trypsin

FIG. 13E—2D Electrophoresis of Human Albumin after pasteurization from Fraction IV

FIG. 13F—2D Electrophoresis of AFCC®

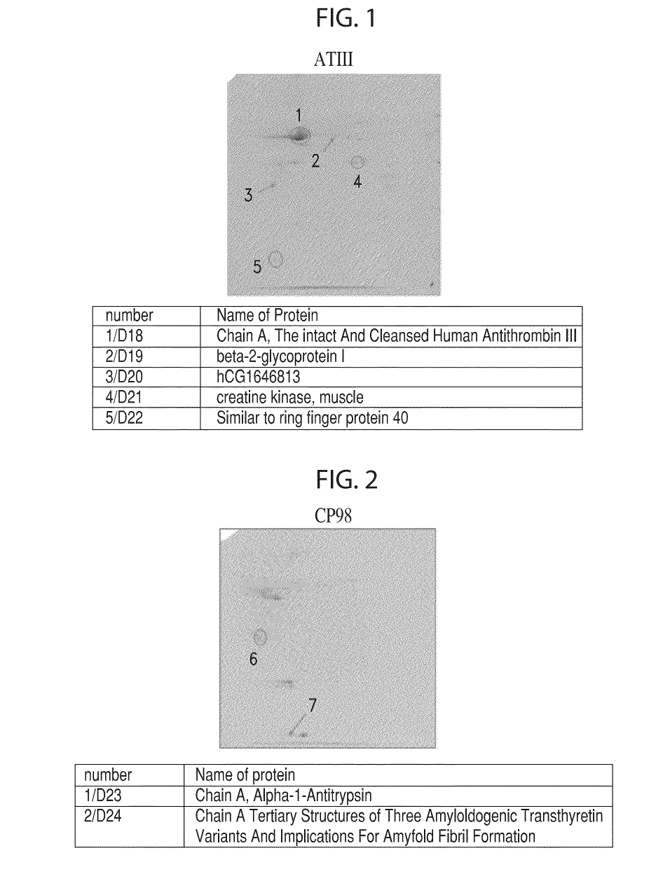

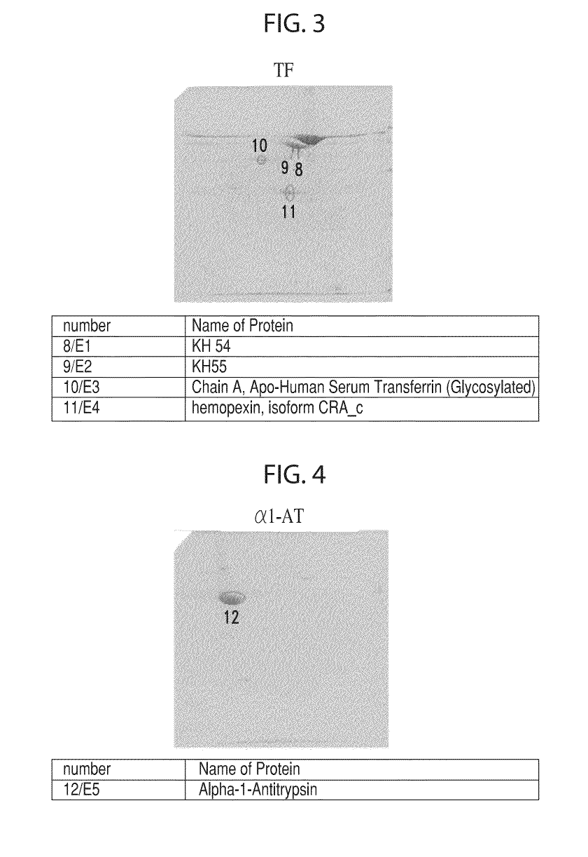

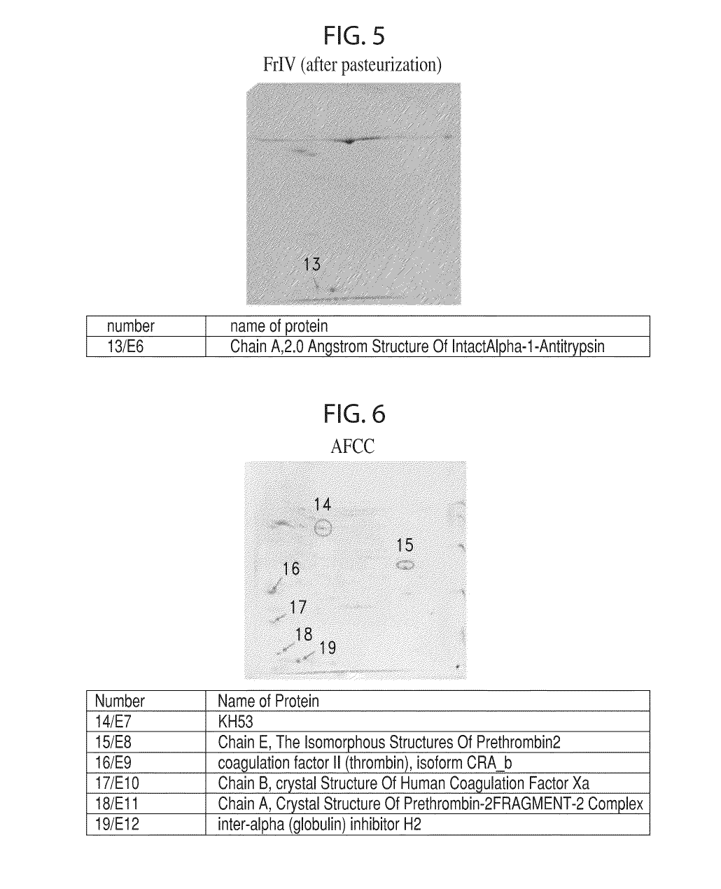

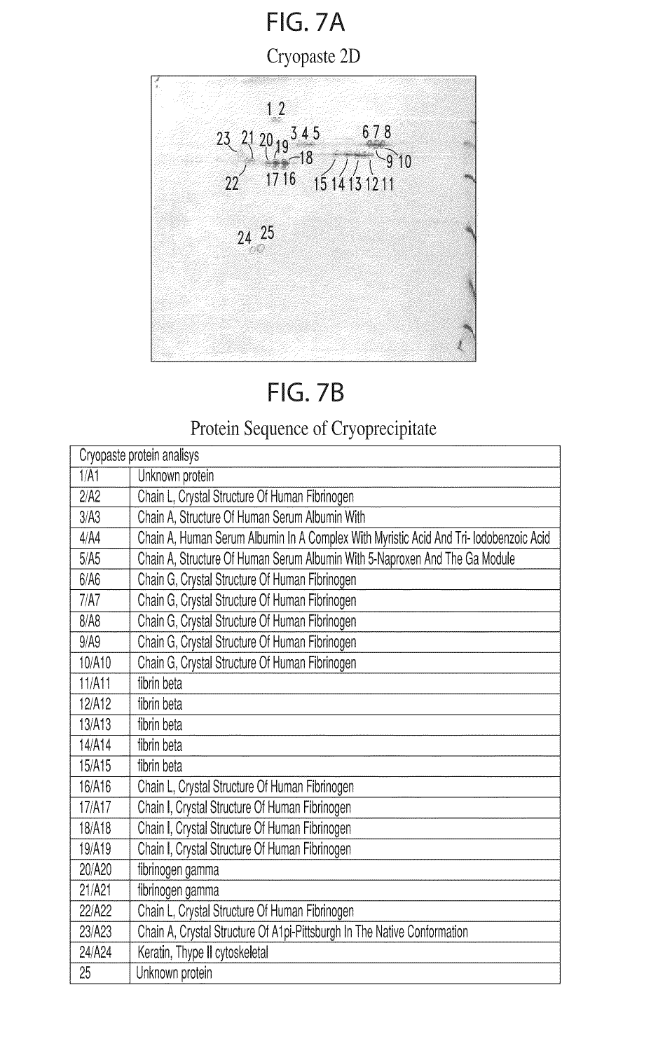

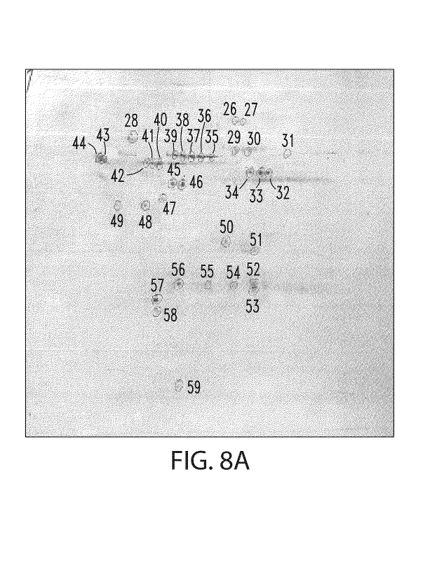

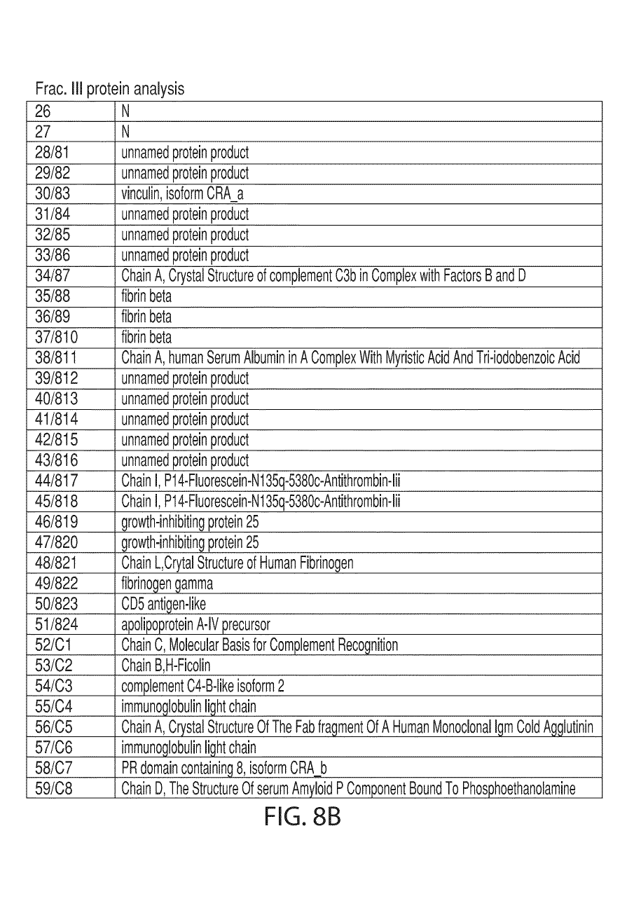

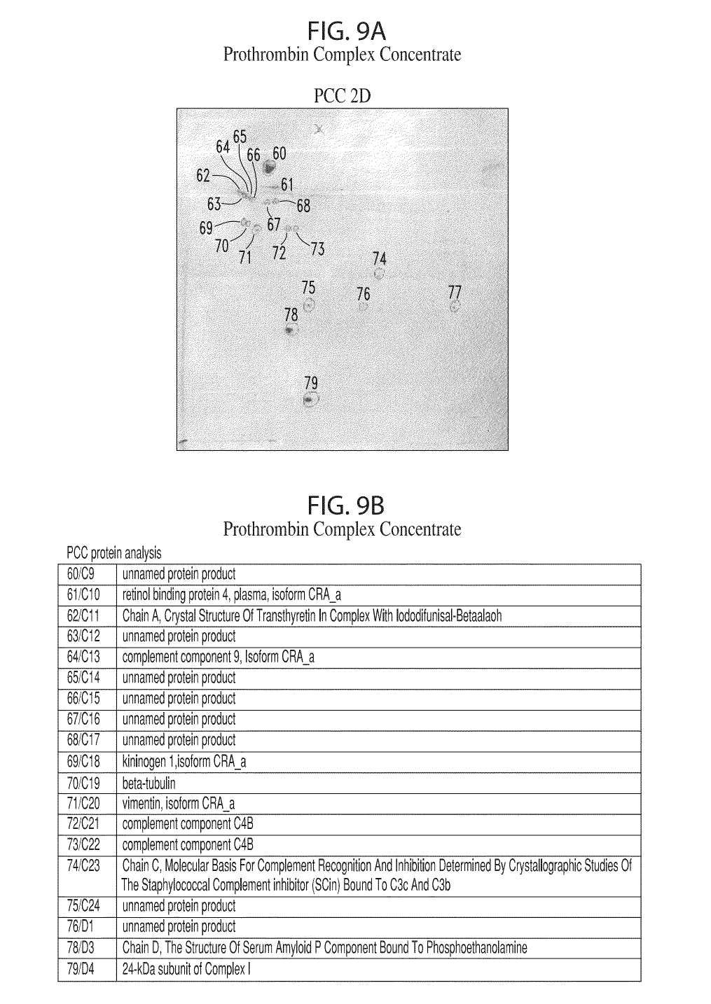

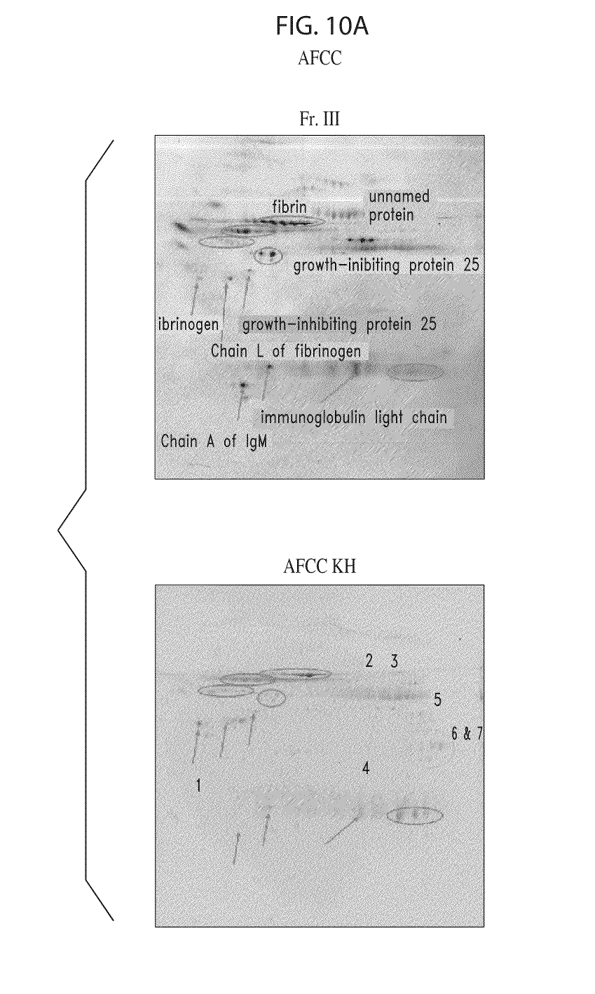

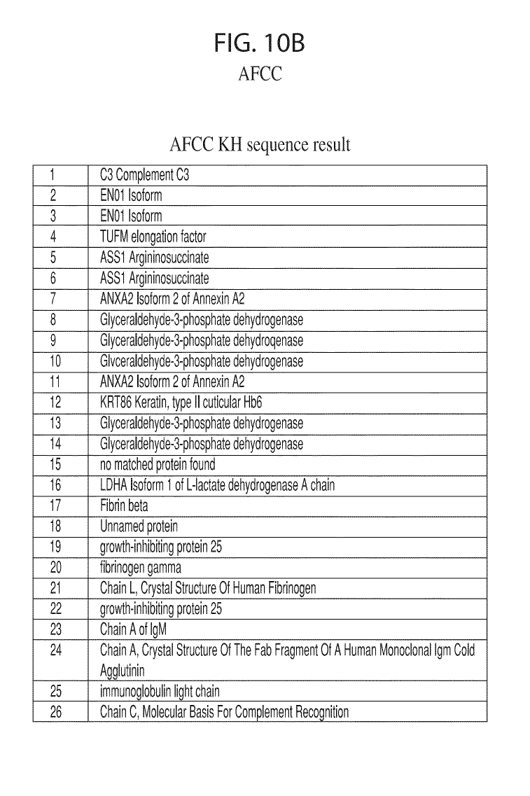

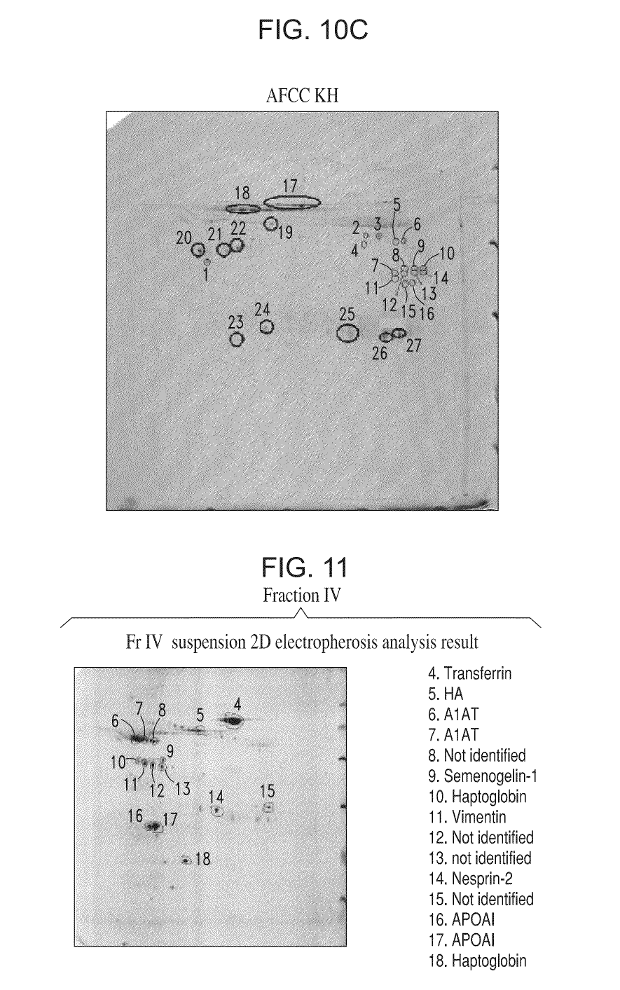

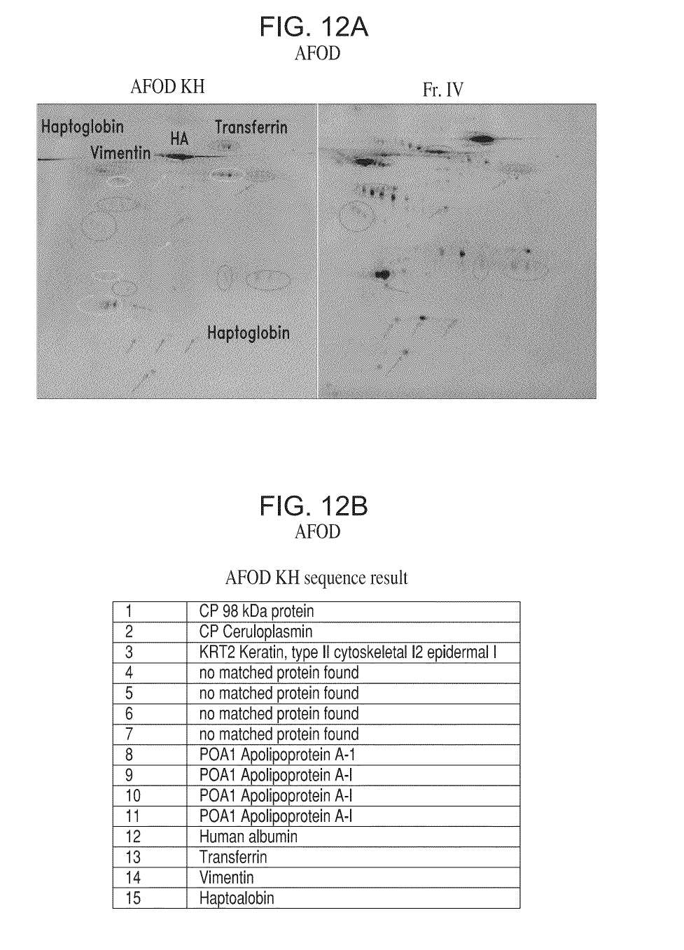

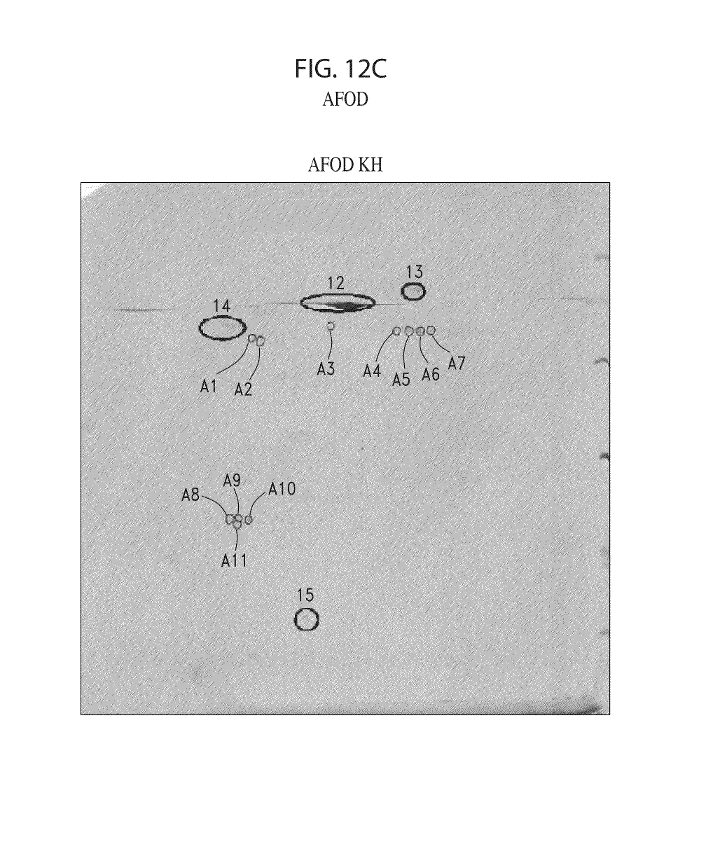

The following are the sequence of these 27 newfound proteins in Cryoprecipitate, Fraction III, Prothrombin Complex Concentrate, AFCC (fraction 33), fraction IV & AFOD (fraction 42).

### 1—Cryoprecipitate

The sequence of these 2 new found proteins and their function are being investigated.

| | | | |
|---|---|---|---|
| | 1/A1 | | Instr./Gel |
| | | | Origin |
| | [1] Sample Project | | Instrument |
| | 20110503 | | Sample |
| | | | Name |
| Protein Name- | KH1 (SEQ ID NO. KH1) | Accession No. | |

| | |
|---|---|
| nitric oxide synthase 1 (neuronal), | gi\|119618515 |
| isoform CRA_b [Homo sapiens] | |

Peptide Information

| Calc. Mass | Obsrv. Mass | ±da | ±ppm | Start Seq. | End Seq. | Sequence |
|---|---|---|---|---|---|---|
| 805.4236 | 805.3599 | −.0637 | −79 | 988 | 994 | IMTQQGK |
| 821.4185 | 821.4248 | .0063 | 8 | 988 | 994 | IMTQQGK |
| 848.4625 | 848.4198 | −.0427 | −50 | 47 | 53 | LQVFDAR |
| 851.4158 | 851.4299 | .0141 | 17 | 881 | 886 | SFWQQR |
| 865.427 | 865.416 | −.011 | −13 | 367 | 374 | LMGQAMAK |
| 865.427 | 865.416 | −.011 | −13 | 367 | 374 | LMGQAMAK |
| 887.4832 | 887.3946 | −.0886 | −100 | 686 | 692 | VELLEER |
| 887.4832 | 887.3946 | −.0886 | −100 | 686 | 692 | VELLEER |
| 899.3629 | 899.3597 | −.0032 | −4 | 1045 | 1052 | DTDEVFSS |
| 903.4166 | 903.3696 | −.047 | −52 | 480 | 488 | SSGDGPDLR |
| 903.4166 | 903.3696 | −.047 | −52 | 480 | 488 | SSGDGPDLR |
| 907.4488 | 907.4044 | −.0444 | −49 | 237 | 243 | MNLDMRK |
| 929.3417 | 929.41 | .0683 | 73 | 220 | 226 | DYCDNSR |
| 931.4955 | 931.4161 | −.0794 | −85 | 341 | 349 | GTNGTPTKR |
| 1037.5231 | 1037.5519 | .0288 | 28 | 367 | 375 | LMGQAMAKR |
| 1103.5439 | 1103.4888 | −.0551 | −50 | 576 | 585 | ANNSLIS |
| | | | | | | NDR |
| 1103.5439 | 1103.4888 | −.0551 | −50 | 576 | 585 | ANNSLIS |
| | | | | | | NDR |
| 1163.5725 | 1163.5321 | −.0404 | −35 | 1 | 9 | MERLEEV |
| | | | | | | NK |
| 1167.6256 | 1167.5096 | −.116 | −99 | 378 | 388 | ATILYATE |
| | | | | | | TGK |

immunoglobulin heavy chain variable region [Homo sapiens gi|10636642]

Peptide Information

| Calc. Mass | Obsrv. Mass | ±da | ±ppm | Start Seq. | End Seq. | Sequence |
|---|---|---|---|---|---|---|
| 815.3981 | 815.3336 | −.0645 | −79 | 1 | 6 | AMHWVR |
| 824.3897 | 824.3629 | −.0268 | −33 | 27 | 33 | NYADSVR |
| 870.4581 | 870.5102 | .0521 | 60 | 34 | 40 | GRFTFSR |
| 891.4352 | 891.429 | −.0062 | −7 | 67 | 74 | ALDCSLGR |
| 1037.5123 | 1037.5519 | .0396 | 38 | 27 | 35 | NYADSVRGR |

| | |
|---|---|
| fibrinogen gamma | gi\|223170 |

Peptide Information

| | | | | Start | | |
|---|---|---|---|---|---|---|
| Calc. Mass | Obsrv. Mass | ±da | ±ppm | Seq. | End Seq. | Sequence |
| 842.4229 | 842.4064 | −.0165 | −20 | 375 | 380 | WYSMKK |
| 842.4229 | 842.4064 | −.0165 | −20 | 375 | 380 | WYSMKK |
| 851.441 | 851.4299 | −.0111 | −13 | 207 | 212 | NWIQYK |
| 909.442 | 909.4585 | .0165 | 18 | 89 | 95 | MLEEIMK |
| 979.5359 | 979.4785 | −.0574 | −59 | 206 | 212 | KNWIQYK |
| 1034.5266 | 1034.531 | .0044 | 4 | 267 | 275 | VGPEADKYR |
| 1037.537 | 1037.5519 | .0149 | 14 | 88 | 95 | KMLEEIMK |
| 1150.5085 | 1150.5156 | .0071 | 6 | 257 | 266 | TSTADYAMFK |
| 1194.4878 | 1194.4916 | .0038 | 3 | 6 | 14 | DNCCILDER |

| | |
|---|---|
| Chain I, Crystal Structure Of Human Fibrinogen | gi\|237823922 |

Protein Group

| | |
|---|---|
| Chain C, Crystal Structure Of Human Fibrinogen | gi\|237823916 |
| Chain F, Crystal Structure Of Human Fibrinogen | gi\|237823919 |
| Chain L, Crystal Structure Of Human Fibrinogen | gi\|237823925 |

Peptide Information

| Calc. Mass | Obsrv. Mass | ±da | ±ppm | Start Seq. | End Seq. | Sequence |
|---|---|---|---|---|---|---|
| 842.4229 | 842.4064 | −.0165 | −20 | 376 | 381 | WYSMKK |
| 842.4229 | 842.4064 | −.0165 | −20 | 376 | 381 | WYSMKK |
| 851.441 | 851.4299 | −.0111 | −13 | 207 | 212 | NWIQYK |
| 909.442 | 909.4585 | .0165 | 18 | 89 | 95 | MLEEIMK |
| 979.5359 | 979.4785 | −.0574 | −59 | 206 | 212 | KNWIQYK |
| 1034.5266 | 1034.531 | .0044 | 4 | 267 | 275 | VGPEADKYR |
| 1037.537 | 1037.5519 | .0149 | 14 | 88 | 95 | KMLEEIMK |

| | |
|---|---|
| fibrinogen gamma chain, isoform | gi\|119625314 |
| CRA_e [Homo sapiens] | |

| Calc. Mass | Obsrv. Mass | ±da | ±ppm | Start | End | Sequence |
|---|---|---|---|---|---|---|
| 842.4229 | 842.4064 | −.0165 | −20 | 384 | 389 | WYSMKK |
| 842.4229 | 842.4064 | −.0165 | −20 | 384 | 389 | WYSMKK |
| 851.441 | 851.4299 | −.0111 | −13 | 215 | 220 | NWIQYK |
| 909.442 | 909.4585 | .0165 | 18 | 115 | 121 | MLEEIMK |
| 979.5359 | 979.4785 | −.0574 | −59 | 214 | 220 | KNWIQYK |
| 1034.5266 | 1034.531 | .0044 | 4 | 275 | 283 | VGPEADKYR |
| 1037.537 | 1037.5519 | .0149 | 14 | 114 | 121 | KMLEEIMK |
| 1150.5085 | 1150.5156 | .0071 | 6 | 265 | 274 | TSTADYAMFK |
| 1194.4878 | 1194.4916 | .0038 | 3 | 32 | 40 | DNCCILDER |

| | |
|---|---|
| fibrinogen gamma chain, isoform | gi\|1196253 |
| sapiens] | |

| Calc. Mass | Obsrv. Mass | ±da | ±ppm | Start Seq. | End Seq. | Sequence |
|---|---|---|---|---|---|---|
| 842.4229 | 842.4064 | −.0165 | −20 | 385 | 390 | WYSMKK |
| 842.4229 | 842.4064 | −.0165 | −20 | 385 | 390 | WYSMKK |
| 851.441 | 851.4299 | −.0111 | −13 | 216 | 221 | NWIQYK |
| 909.442 | 909.4585 | .0165 | 18 | 98 | 104 | MLEEIMK |
| 979.5359 | 979.4785 | −.0574 | −59 | 215 | 221 | KNWIQYK |
| | 979.4785 | | | | | |
| 1034.5266 | 1034.531 | .0044 | 4 | 276 | 284 | VGPEADKYR |
| 1037.537 | 1037.5519 | .0149 | 14 | 97 | 104 | KMLEEIMK |
| 1150.5085 | 1150.5156 | .0071 | 6 | 266 | 275 | TSTADYAMFK |
| 1194.4878 | 1194.4916 | .0038 | 3 | 15 | 23 | DNCCILDER |

| | |
|---|---|
| fibrinogen gamma chain, isoform | gi\|119625320 |
| GRA_j [Homo sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 842.4229 | 842.4064 | −.0165 | −20 | 387 | 392 | WYSMKK |
| 842.4229 | 842.4064 | −.0165 | −20 | 387 | 392 | WYSMKK |
| 851.441 | 851.4299 | −.0111 | −13 | 233 | 238 | NWIQYK |
| 909.442 | 909.4585 | .0165 | 18 | 115 | 121 | MLEEIMK |
| 979.5359 | 979.4785 | −.0574 | −59 | 232 | 238 | KNWIQYK |
| 1034.5266 | 1034.531 | .0044 | 4 | 293 | 301 | VGPEADKYR |
| 1037.537 | 1037.5519 | .0149 | 14 | 114 | 121 | KMLEEIMK |
| 1150.5085 | 1150.5156 | .0071 | 6 | 283 | 292 | TSTADYAMFK |
| 1194.4878 | 1194.4916 | .0038 | 3 | 32 | 40 | DNCCILDER |

Protein Group

| | |
|---|---|
| Chain C, A Fragment Of Streptococcal M1 Protein In | gi\|328877090 |
| Complex With Human Fibrinogen | |
| Chain C, Bc1 Fragment Of Streptococcal M1 Protein In | gi\|328877076 |
| Complex With Human Fibrinogen | |
| Chain C, Crystal Structure Of Bbetad432a Variant | gi\|220702428 |
| Fibrinogen Fragment D With The Peptide Ligand | |
| Gly-His-Arg-Pro-Amide | |
| Chain C, Crystal Structure Of Crosslinked Fragment D | gi\|2781215 |
| Chain C, Crystal Structure Of Fibrinogen Fragment D | gi\|2781209 |
| Chain C, Crystal Structure Of Fragment D From Human | gi\|5542261 |
| Fibrinogen With The Peptide Ligand | |
| Gly-His-Arg-Pro-Amide | |
| Chain C, Crystal Structure Of Fragment Double-D From | gi\|5542245 |
| Human | |
| Fibrin | |
| Chain C, Crystal Structure Of Fragment Double-D From | gi\|5542251 |
| Human Fibrin With The Peptide Ligand | |
| Gly-His-Arg-Pro-Amide | |
| Chain C, Crystal Structure Of Fragment Double-D From | gi\|3891882 |
| Human Fibrin With Two Different Bound Ligands | |
| Chain F, A Fragment Of Streptococcal M1 Protein In | gi\|328877093 |
| Complex With Human Fibrinogen | |
| Chain F, Bc1 Fragment Of Streptococcal M1 Protein In | gi\|328877079 |
| Complex With Human Fibrinogen | |
| Chain F, Crystal Structure Of Bbetad432a Variant | gi\|220702431 |
| Fibrinogen Fragment D With The Peptide Ligand | |
| Gly-His-Arg-Pro-Amide | |
| Chain F, Crystal Structure Of Crosslinked Fragment D | gi\|2781218 |
| Chain F, Crystal Structure Of Fibrinogen Fragment D | gi\|2781212 |
| Chain F, Crystal Structure Of Fragment D From Human | gi\|5542264 |
| Gly-His-Arg-Pro-Amide | |
| Chain F, Crystal Structure Of Fragment Double-D From | gi\|5542248 |
| Human Fibrin | |
| Human Fibrin With The Peptide Ligand | |
| Gly-His-Arg-Pro-Amide | |
| Chain F, Crystal Structure Of Fragment Double-D From | gi\|3891885 |
| Human Fibrin With Two Different Bound Ligands | |
| Complex With Human Fibrinogen | |
| Complex With Human Fibrinogen | |

Peptide Information

| | | | | Start | | |
|---|---|---|---|---|---|---|
| Calc. Mass | Obsrv. Mass | ±da | ±ppm | Seq. | End Seq. | Sequence |
| 842.4229 | 842.4064 | −.0165 | −20 | 289 | 294 | WYSMKK |
| 842.4229 | 842.4064 | −.0165 | −20 | 289 | 294 | WYSMKK |
| 851.441 | 851.4299 | −.0111 | −13 | 120 | 125 | NWIQYK |
| 909.442 | 909.4585 | .0165 | 18 | 2 | 8 | MLEEIMK |
| 979.5359 | 979.4785 | −.0574 | −59 | 119 | 125 | KNWIQYK |
| 1034.5266 | 1034.531 | .0044 | 4 | 180 | 188 | VGPEADKYR |
| 1037.537 | 1037.5519 | .0149 | 14 | 1 | 8 | KMLEEIMK |
| 1150.5085 | 1150.5156 | .0071 | 6 | 170 | 179 | TSTADYAMFK |

| | |
|---|---|
| nitric oxide synthase 1 (neuronal), isoform | gi\|119618514 |
| CRA_a [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 805.4236 | 805.3599 | −.0637 | −79 | 1022 | 1028 | IMTQQGK |
| 821.4185 | 821.4248 | .0063 | 8 | 1022 | 1028 | IMTQQGK |
| 848.4625 | 848.4198 | −.0427 | −50 | 47 | 53 | LQVFDAR |
| 851.4158 | 851.4299 | .0141 | 17 | 915 | 920 | SFWQQR |
| 865.427 | 865.416 | −.011 | −13 | 367 | 374 | LMGQAMAK |
| 865.427 | 865.416 | −.011 | −13 | 367 | 374 | LMGQAMAK |
| 887.4832 | 887.3946 | −.0886 | −100 | 720 | 726 | VELLEER |
| 887.4832 | 887.3946 | −.0886 | −100 | 720 | 726 | VELLEER |
| 899.3629 | 899.3597 | −.0032 | −4 | 1079 | 1086 | DTDEVFSS |
| 903.4166 | 903.3696 | −.047 | −52 | 514 | 522 | SSGDGP |
| | | | | | | DLR |
| 903.4166 | 903.3696 | −.047 | −52 | 514 | 522 | SSGDGP |
| | | | | | | DLR |
| 907.4488 | 907.4044 | −.0444 | −49 | 237 | 243 | MNLDMRK |
| 929.3417 | 929.41 | .0683 | 73 | 220 | 226 | DYCDNSR |
| 931.4955 | 931.4161 | −.0794 | −85 | 341 | 349 | GTNGTPT |
| | | | | | | KR |
| 1037.5231 | 1037.5519 | .0288 | 28 | 367 | 375 | LMGQAM |
| | | | | | | AKR |
| 1103.5439 | 1103.4888 | −.0551 | −50 | 610 | 619 | ANNSLIS |
| | | | | | | NDR |
| 1103.5439 | 1103.4888 | −.0551 | −50 | 610 | 619 | ANNSLIS |
| | | | | | | NDR |
| 1163.5725 | 1163.5321 | −.0404 | −35 | 1 | 9 | MERLEEV |
| | | | | | | NK |
| 1167.6256 | 1167.5096 | −.116 | −99 | 378 | 388 | ATILYATE |
| | | | | | | TGK |

| | |
|---|---|
| fibrinogen gamma chain isoform | gi\|70906437 |
| gamma-A precursor | |
| [Homo sapiens] | |

Protein Group

| | |
|---|---|
| Fibrinogen gamma chain [Homo sapiens] | gi\|18203820 |
| Fibrinogen gamma chain [Homo sapiens] | gi\|13937874 |
| PRO2061 [Homo sapiens] | gi\|6650830 |
| fibrinogen gamma chain, isoform CRA_m | gi\|1196253 |
| sapiens] | |
| [Homo sapiens] | |
| fibrinogen, gamma polypeptide [Homo | |
| sapiens] | |
| unnamed protein product [Homo sapiens] | gi\|158255084 |

Peptide Information

| Calc. | Obsrv. | | | Start | End | |
|---|---|---|---|---|---|---|
| Mass | Mass | ±da | ±ppm | Seq. | Seq. | Sequence |
| 842.4229 | 842.4064 | −.0165 | −20 | 402 | 407 | WYSMKK |
| 842.4229 | 842.4064 | −.0165 | −20 | 402 | 407 | WYSMKK |
| 851.441 | 851.4299 | −.0111 | −13 | 233 | 238 | NWIQYK |
| 909.442 | 909.4585 | .0165 | 18 | 115 | 121 | MLEEIMK |
| 979.5359 | 979.4785 | −.0574 | −59 | 232 | 238 | KNWIQYK |
| 1034.5266 | 1034.531 | .0044 | 4 | 293 | 301 | VGPEADK |
| | | | | | | YR |
| 1037.537 | 1037.5519 | .0149 | 14 | 114 | 121 | KMLEEIMK |
| 1150.5085 | 1150.5156 | .0071 | 6 | 283 | 292 | TSTADYA |
| | | | | | | MFK |
| 1194.4878 | 1194.4916 | .0038 | 3 | 32 | 40 | DNCCILD |
| | | | | | | ER |

| | |
|---|---|
| hCG2000669, isoform CRA_a | gi\|119615136 |
| [Homo sapiens] | |

Protein Group

| | |
|---|---|
| Homeobox B6 [Homo sapiens] | gi\|15779175 |
| RecName: Full = Homeobox protein Hox-B6; AltName: | gi\|116242515 |
| Full = Homeobox protein Hox-2.2; | |
| AltName: Full = Homeobox protein | |
| Hox-2B; AltName: Full = Homeobox | |
| protein | |
| Hu-2 | |
| hCG2000669, isoform CRA_a [Homo sapiens] | gi\|119615135 |
| hCG2000669, isoform CRA_a [Homo sapiens] | gi\|119615137 |
| hCG2000669, isoform CRA_a [Homo sapiens] | gi\|119615134 |
| homeobox B6 [Homo sapiens] | gi\|11138932 |
| homeobox protein Hox-B6 [Homo sapiens] | gi\|23503237 |
| homeobox protein [Homo sapiens] | gi\|6706217 |
| unnamed protein product [Homo sapiens] | gi\|158257832 |

Peptide Information

| Calc. | Obsrv. | | | Start | End | |
|---|---|---|---|---|---|---|
| Mass | Mass | ±da | ±ppm | Seq. | Seq. | Sequence |
| 823.3251 | 823.3582 | .0331 | 40 | 106 | 112 | SDCAQDK |
| 863.4522 | 863.4052 | −.047 | −54 | 192 | 197 | IWFQNR |
| 865.3951 | 865.416 | .0209 | 24 | 164 | 169 | EFHYNR |
| 865.3951 | 865.416 | .0209 | 24 | 164 | 169 | EFHYNR |
| 951.42 | 951.4047 | −.0153 | −16 | 105 | 112 | KSDCAQDK |
| 951.42 | 951.4047 | −.0153 | −16 | 105 | 112 | KSDCAQDK |
| 1019.5533 | 1019.4705 | −.0828 | −81 | 192 | 198 | IWFQNRR |
| 1023.5356 | 1023.5074 | −.0282 | −28 | 156 | 163 | YQTLELEK |

| | |
|---|---|
| Chain F, Crystal Structure Of D-Dimer | gi\|162330006 |
| From Human Fibrin Complexed | |
| With Met-His-Arg-Pro-Tyr-Amide. | |

Peptide Information

| | | | | Start | | |
|---|---|---|---|---|---|---|
| Calc. Mass | Obsrv. Mass | ±da | ±ppm | Seq. | End Seq. | Sequence |
| 842.4229 | 842.4064 | −.0165 | −20 | 289 | 294 | WYSMKK |
| 842.4229 | 842.4064 | −.0165 | −20 | 289 | 294 | WYSMKK |
| 851.441 | 851.4299 | −.0111 | −13 | 120 | 125 | NWIQYK |
| 909.442 | 909.4585 | .0165 | 18 | 2 | 8 | MLEEIMK |
| 979.5359 | 979.4785 | −.0574 | −59 | 119 | 125 | KNWIQYK |
| 1034.5266 | 1034.531 | .0044 | 4 | 180 | 188 | VGPEADKYR |
| 1037.537 | 1037.5519 | .0149 | 14 | 1 | 8 | KMLEEIMK |
| 1150.5085 | 1150.5156 | .0071 | 6 | 170 | 179 | TSTADYAMFK |

| | |
|---|---|
| inactive phospholipase D5 isoform 1 | gi\|307775443 |
| [Homo sapiens] | |

Protein Group

| | |
|---|---|
| PLDc protein [Homo sapiens] | gi\|46392519 |
| Phospholipase D family, member 5 [Homo sapiens] | gi\|71682896 |
| Phospholipase D family, member 5 [Homo sapiens] | gi\|71682143 |
| Phospholipase D family, member 5 [Homo sapiens] | gi\|71682147 |
| RecName: Full = Inactive phospholipase | |
| D5; | |
| Short = Inactive PLD 5; AltName: Full = Inactive | gi\|152112223 |
| choline | |
| phosphatase 5; AltName: Full = Inactive | |
| phosphatidylcholine-hydrolyzing | |
| phospholipase D5; AltName: Full = PLDc | |
| inactive phospholipase D5 isoform 2 [Homo sapiens] | gi\|307775435 |
| inactive phospholipase D5 isoform 3 [Homo sapiens] | gi\|307775437 |
| phospholipase D family, member 5 [Homo sapiens] | \|55960909 |
| phospholipase D family, member 5 [Homo sapiens] | gi\|55960908 |
| phospholipase D family, member 5 [Homo sapiens] | gi\|55665367 |
| phospholipase D family, member 5 [Homo sapiens] | gi\|55959384 |
| phospholipase D family, member 5, isoform | gi\|119590518 |
| CRA_a [Homo sapiens] | |
| phospholipase D family, member 5, isoform | gi\|119590517 |
| CRA_a [Homo sapiens] | |
| phospholipase D family, member 5, isoform | gi\|119590519 |
| CRA_b [Homo sapiens] | |
| unnamed protein product [Homo sapiens] | gi\|221040766 |
| unnamed protein product [Homo sapiens] | gi\|21758029 |
| unnamed protein product [Homo sapiens] | gi\|21750120 |

Peptide Information

| Calc. | Obsrv. | | | Start | End | |
|---|---|---|---|---|---|---|
| Mass | Mass | ±da | ±ppm | Seq. | Seq. | Sequence |
| 865.3898 | 865.416 | .0262 | 30 | 523 | 531 | TATDDTGGK |

| | |
|---|---|
| RecName: Full = Zinc finger | gi\|145559547 |
| protein 493 | |

Protein Group

| | |
|---|---|
| LDL induced EC protein [Homo sapiens] | gi\|5924319 |
| LDL induced endothelial cell protein | gi\|8518179 |
| [Homo sapiens] | |
| RecName: Full = Zinc finger protein 580; | gi\|55976777 |
| AltName: Full = LDL-induced EC | |
| protein | |
| ZNF493 protein [Homo sapiens] | gi\|85726510 |
| Zinc finger protein 580 [Homo sapiens] | gi\|17389292 |
| similar to hypothetical protein FLJ13659, | gi\|119605302 |
| isoform CRA_a [Homo sapiens] | |
| similar to hypothetical protein FLJ13659, | gi\|119605305 |
| isoform CRA_d [Homo sapiens] | |
| unnamed protein product [Homo sapiens] | gi\|189054604 |
| unnamed protein product [Homo sapiens] | gi\|21754914 |
| unnamed protein product [Homo sapiens] | gi\|34534405 |
| unnamed protein product [Homo sapiens] | gi\|34535891 |
| zinc finger protein 493 isoform 1 [Homo sapiens] | gi\|115511042 |
| zinc finger protein 493 isoform 3 [Homo sapiens] | gi\|115511044 |
| zinc finger protein 493 variant [Homo sapiens] | gi\|62087752 |
| zinc finger protein 580 [Homo sapiens] | gi\|7705881 |
| zinc finger protein 580 [Homo sapiens] | gi\|119592805 |
| zinc finger protein 580 [Homo sapiens] | gi\|254540128 |
| zinc finger protein 580 [Homo sapiens] | gi\|46361970 |

Peptide Information

| | Obsrv. | | | Start | End | |
|---|---|---|---|---|---|---|
| Calc. Mass | Mass | ±da | ±ppm | Seq. | Seq. | Sequence |
| 842.4591 | 842.4064 | −.0527 | −63 | 624 | 630 | RSSHLSR |
| | 842.4064 | | | | | |

| | |
|---|---|
| hCG2015903 [Homo sapiens] | gi\|119616226 |

Peptide Information

| | Obsrv. | | | Start | End | |
|---|---|---|---|---|---|---|
| Calc. Mass | Mass | ±da | ±ppm | Seq. | Seq. | Sequence |
| 842.4591 | 842.4064 | −.0527 | −63 | 44 | 50 | RSHSVTR |

RecName: Full = Klotho; Contains: RecName:
Full = Klotho peptide; Flags: Precursor

Protein Group

| | |
|---|---|
| | gi\|77416517 |
| 179I15.1 (klotho) [Homo sapiens] | gi\|16116628 |
| Klotho protein (KL) [Homo sapiens] | gi\|2924562 |
| Klotho protein (KL) [Homo sapiens] | gi\|2924561 |
| klotho [Homo sapiens] | gi\|55663139 |
| klotho [Homo sapiens] | gi\|2618596 |
| klotho precursor [Homo sapiens] | gi\|24497614 |
| klotho, isoform CRA_a [Homo sapiens] | gi\|119628930 |
| klotho, isoform CRA_b [Homo sapiens] | gi\|119628931 |
| unnamed protein product [Homo sapiens] | gi\|209985373 |
| unnamed protein product [Homo sapiens] | gi\|209985371 |

Peptide Information

| | Obsrv. | | | Start | End | |
|---|---|---|---|---|---|---|
| Calc. Mass | Mass | ±da | ±ppm | Seq. | Seq. | Sequence |
| 842.4189 | 842.4064 | −.0125 | −15 | 1 | 8 | MPASAPPR |

| | |
|---|---|
| unnamed protein product [Homo sapiens] | gi\|40039486 |

Peptide Information

| Calc. | | | | Star | End | |
|---|---|---|---|---|---|---|
| Mass | Obsrv. | ±da | ±ppm | Seq. | Seq. | Sequence |
| 1103.515 | 1103.4888 | −.0262 | −24 | 58 | 68 | IPGSPXESMGR |

| | |
|---|---|
| Chain A, Crystal Structure Of The Small | gi\|1193890 |
| (Gemdndcam) In Complex To Mg.Gdp | |

Protein Group

| | |
|---|---|
| Chain B, Crystal Structure Of The Small | gi\|1193890 |
| (Gemdndcam) In Complex To Mg.Gdp | |

Peptide Information

| | Obsrv. | | | | | |
|---|---|---|---|---|---|---|
| Calc. Mass | Mass | ±da | ±ppm | Star | End | Sequence |
| 1149.5681 | 1149.517 | −.0511 | −44 | 12 | 138 | XREVSVSEGR |

| | |
|---|---|
| coiled-coil domain-containing protein | gi\|2245932 |
| 60 | |

Protein Group

| | |
|---|---|
| Coiled-coil domain containing 60 [Homo | gi\|2625179 |
| RecName: Full = Coiled-coil domain-containing | gi\|296434427 |
| protein | |
| 60 | |
| coiled-coil domain containing 60, isoform | gi\|119618556 |
| CRA_d [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 1103.599 | 1103.4888 | −.1102 | −100 | 222 | 230 | IPTMRVTNR |

| | |
|---|---|
| unnamed protein product [Homo sapiens] | gi\|21749960 |

Peptide Information

| Calc. | Obsrv. | | | Start | End | |
|---|---|---|---|---|---|---|
| Mass | Mass | ±da | ±ppm | Seq. | Seq. | Sequence |
| 1103.4747 | 1103.4888 | .0141 | 13 | 646 | 654 | MMEASLY |
| | | | | | | DK |

| | | | |
|---|---|---|---|
| | 24/A24 | | Instr./Gel |
| | | | Origin |
| | [1] Sample Project | | Instrument |
| | 20110503 | | Sample |
| | | | Name |
| Protein Name- | KH2 (SEQ ID NO. KH2) | Accession No. | |

| | |
|---|---|
| keratin, type II cytoskeletal 2 | gi\|47132620 |
| epidermal [Homo sapiens] | |
| Protein Group | |
| Keratin 2 [Homo sapiens] | gi\|68563400 |
| Keratin 2 [Homo sapiens] | gi\|68563334 |
| Keratin 2 [Homo sapiens] | |
| Keratin 2 [Homo sapiens] | gi\|64654451 |

RecName: Full = Keratin, type II
cytoskeletal 2
epidermal; AltName: Full = Cytokeratin-2e; Short = CK-2e;
AltName: Full = Epithelial keratin-2e; AltName:
Full = Keratin-2 epidermis; AltName: Full = Keratin-2e;

| | |
|---|---|
| Short = K2e; AltName: Full = | gi\|239938650 |
| Type-II keratin Kb2 | |
| keratin 2A (epidermal ichthyosis bullosa | gi\|119617047 |
| of Siemens) [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 831.4935 | 831.4731 | −.0204 | −25 | 62 | 70 | SLVGLGGTK |
| 996.5221 | 996.6213 | .0992 | 100 | 431 | 439 | GEHALKDAR |
| 1001.5374 | 1001.5252 | −.0122 | −12 | 176 | 183 | AQEREQIK |
| 1107.543 | 1107.5515 | .0085 | 8 | 354 | 362 | AQYEEIAQR |
| 1254.6074 | 1254.649 | .0416 | 33 | 21 | 34 | GFSSGSAVVSGGSR |
| 1272.5493 | 1272.5642 | .0149 | 12 | 1 | 10 | MSCQISCKSR |
| 1320.5829 | 1320.6151 | .0322 | 24 | 46 | 61 | HGGGGGGFGGGG |
| | | | | | | FGSR |
| 1320.5829 | 1320.6151 | .0322 | 24 | 46 | 61 | HGGGGGGFGGGG |
| | | | | | | FGSR |
| 1740.7057 | 1740.7317 | .026 | 15 | 525 | 544 | GSSSGGGYSSGSS |
| | | | | | | SYGS |
| | | | | | | GGR |
| 1741.7485 | 1741.7495 | .001 | 1 | 550 | 572 | GGSGGGGSISGGG |
| | | | | | | YGS |
| | | | | | | GGGSGGR |
| 1752.8837 | 1752.7648 | −.1189 | −68 | 288 | 302 | DVDNAYMIKVELQSK |
| 1838.9144 | 1838.9127 | −.0017 | −1 | 71 | 92 | SISISVAGGGGGFG |
| | | | | | | AAG |
| | | | | | | GFGGR |

| | |
|---|---|
| keratin 2e [Homo sapiens] | gi\|2565257 |

Protein Group

| | |
|---|---|
| epidermal cytokeratin 2 [Homo sapiens] | gi\|181402 |

Peptide Information

| | | | | Start | End | |
|---|---|---|---|---|---|---|
| Calc. Mass | Obsrv. Mass | ±da | ±ppm | Seq. | Seq. | Sequence |
| 831.4935 | 831.4731 | −.0204 | −25 | 62 | 70 | SLVGLGGTK |
| 996.5221 | 996.6213 | .0992 | 100 | 437 | 445 | GEHALKDAR |
| 1001.5374 | 1001.5252 | −.0122 | −12 | 182 | 189 | AQEREQIK |
| 1107.543 | 1107.5515 | .0085 | 8 | 360 | 368 | AQYEEIAQR |
| 1254.6074 | 1254.649 | .0416 | 33 | 21 | 34 | GFSSGSAVVSGGSR |
| 1272.5493 | 1272.5642 | .0149 | 12 | 1 | 10 | MSCQISCKSR |
| 1320.5829 | 1320.6151 | .0322 | 24 | 46 | 61 | HGGGGGGFGG |
| | | | | | | GGFGSR |
| 1320.5829 | 1320.6151 | .0322 | 24 | 46 | 61 | HGGGGGGFGG |
| | | | | | | GGFGSR |
| 1740.7057 | 1740.7317 | .026 | 15 | 531 | 550 | GSSSGGGYSSG |
| | | | | | | SSSYGS |
| | | | | | | GGR |
| 1741.7485 | 1741.7495 | .001 | 1 | 556 | 578 | GGSGGGGSISG |
| | | | | | | GGYGS |
| | | | | | | GGGSGGR |
| 1752.8837 | 1752.7648 | −.1189 | −68 | 294 | 308 | DVDNAYMIKVEL |
| | | | | | | QSK |
| 1838.9144 | 1838.9127 | −.0017 | −1 | 71 | 92 | SISISVAGGGGG |
| | | | | | | FGAAG |
| | | | | | | GFGGR |

| | |
|---|---|
| unnamed protein product [Homo sapiens] | gi\|215415640 |

Peptide Information

| Calc. | Obsrv. | | | Start | End | |
|---|---|---|---|---|---|---|
| Mass | Mass | ±da | ±ppm | Seq. | Seq. | Sequence |
| 831.4359 | 831.4731 | .0372 | 45 | 189 | 195 | LAEYHAK |
| 1012.5786 | 1012.5838 | .0052 | 5 | 207 | 215 | AKPALEDL |
| | | | | | | R |
| 1226.5437 | 1226.5537 | .01 | 8 | 1 | 10 | DEPPQSPW |
| | | | | | | DR |
| 1226.5437 | 1226.5537 | .01 | 8 | 1 | 10 | DEPPQSPW |
| | | | | | | DR |
| 1301.6484 | 1301.6486 | .0002 | 0 | 161 | 171 | THLAPYSD |
| | | | | | | ELR |
| 1301.6484 | 1301.6486 | .0002 | 0 | 161 | 171 | THLAPYSD |
| | | | | | | ELR |

| | |
|---|---|
| Chain A, Solution Structure Of Double | gi\|293651900 |
| Super Helix Model | |

Protein Group

| | |
|---|---|
| Chain A, Crystal Structure Of Lipid-Free Human | gi\|90108664 |
| Apolipoprotein A-I | |
| Chain B, Crystal Structure Of Lipid-Free Human | gi\|90108665 |
| Apolipoprotein A-I | |
| Chain B, Solution Structure Of Double Super Helix | gi\|293651901 |
| Model | |
| Chain C, Crystal Structure Of Lipid-Free Human | gi\|90108666 |
| Apolipoprotein A-I | |
| apoA1 [Homo sapiens] | gi\|490098 |
| apoAI [Homo sapiens] | gi\|1330306 |
| unnamed protein product [Homo sapiens] | gi\|215415678 |
| unnamed protein product [Homo sapiens] | gi\|215415656 |
| unnamed protein product [Homo sapiens] | |
| unnamed protein product [Homo sapiens] | gi\|215415652 |
| unnamed protein product [Homo sapiens] | gi\|215415650 |
| unnamed protein product [Homo sapiens] | gi\|215415648 |
| unnamed protein product [Homo sapiens] | gi\|215415644 |
| unnamed protein product [Homo sapiens] | gi\|215415638 |
| unnamed protein product [Homo sapiens] | gi\|215415636 |
| unnamed protein product [Homo sapiens] | gi\|215415626 |
| unnamed protein product [Homo sapiens] | gi\|215415624 |

Peptide Information

| Calc. | Obsrv. | | | Start | End | |
|---|---|---|---|---|---|---|
| Mass | Mass | ±da | ±ppm | Seq. | Seq. | Sequence |
| 831.4359 | 831.4731 | .0372 | 45 | 189 | 195 | LAEYHAK |
| 1012.5786 | 1012.5838 | .0052 | 5 | 207 | 215 | AKPALEDL |
| | | | | | | R |
| 1226.5437 | 1226.5537 | .01 | 8 | 1 | 10 | DEPPQSPW |
| | | | | | | DR |
| 1226.5437 | 1226.5537 | .01 | 8 | 1 | 10 | DEPPQSPW |
| | | | | | | DR |
| 1301.6484 | 1301.6486 | .0002 | 0 | 161 | 171 | THLAPYSD |
| | | | | | | ELR |
| 1301.6484 | 1301.6486 | .0002 | 0 | 161 | 171 | THLAPYSD |
| | | | | | | ELR |

| | |
|---|---|
| lipoprotein Gln I | gi\|229479 |

Protein Group

| | |
|---|---|
| protein, lipid binding AI | gi\|229513 |

Peptide Information

| Calc. | Obsrv. | | | Start | End | |
|---|---|---|---|---|---|---|
| Mass | Mass | ±da | ±ppm | Seq. | Seq. | Sequence |
| 831.4359 | 831.4731 | .0372 | 45 | 192 | 198 | LAEYHAK |
| 1012.5786 | 1012.5838 | .0052 | 5 | 210 | 218 | AKPALEDL |
| | | | | | | R |
| 1226.5437 | 1226.5537 | .01 | 8 | 1 | 10 | DEPPQSP |
| | | | | | | WDR |
| 1226.5437 | 1226.5537 | .01 | 8 | 1 | 10 | DEPPQSP |
| | | | | | | WDR |
| 1301.6484 | 1301.6486 | .0002 | 0 | 164 | 174 | THLAPYS |
| | | | | | | DELR |
| 1301.6484 | 1301.6486 | .0002 | 0 | 164 | 174 | THLAPYS |
| | | | | | | DELR |

| | |
|---|---|
| chromodomain helicase DNA binding | gi\|119603201 |
| protein 9, isoform | |
| CRA_c [Homo sapiens] | |

Peptide Information

| | Obsrv. | | | Start | | |
|---|---|---|---|---|---|---|
| Calc. Mass | Mass | ±da | ±ppm | Seq. | End Seq. | Sequence |
| 800.4876 | 800.43 | −.0576 | −72 | 1437 | 1444 | DLLIGAAK |
| 802.457 | 802.4542 | −.0028 | −3 | 1067 | 1072 | AEWLRK |
| 806.4254 | 806.4277 | .0023 | 3 | 42 | 48 | KVESESK |
| 807.3843 | 807.409 | .0247 | 31 | 2283 | 2290 | NSDLGSSK |
| 854.5458 | 854.5139 | −.0319 | −37 | 2069 | 2076 | ILTGPVVR |
| 856.4999 | 856.5127 | .0128 | 15 | 9 | 16 | GRPPSSKK |
| 887.4832 | 887.4777 | −.0055 | −6 | 819 | 825 | EIEDLLR |
| 903.4828 | 903.4889 | .0061 | 7 | 1729 | 1735 | DRVMINR |
| 917.4839 | 917.4818 | −.0021 | −2 | 1450 | 1456 | TDYHILR |
| 932.5121 | 932.4993 | −.0128 | −14 | 1507 | 1514 | GIILEEMK |
| 932.5121 | 932.4993 | −.0128 | −14 | 1507 | 1514 | GIILEEMK |
| 948.4931 | 948.4807 | −.0124 | −13 | 797 | 805 | AVLQSMSGR |
| 960.5407 | 960.5235 | −.0172 | −18 | 1 | 8 | MSSVKRPR |
| 1012.5105 | 1012.5838 | .0733 | 72 | 760 | 767 | CHRIGQNK |
| 1107.5542 | 1107.5515 | −.0027 | −2 | 321 | 329 | DYKNGNQLR |
| 1185.6949 | 1185.6342 | −.0607 | −51 | 52 | 62 | ANRIISEAIAK |
| 1191.5826 | 1191.6141 | .0315 | 26 | 1168 | 1177 | ADPALCFLER |
| 1198.7042 | 1198.6854 | −.0188 | −16 | 1539 | 1549 | TLIKSEPVSPK |
| 1301.7059 | 1301.6486 | −.0573 | −44 | 897 | 908 | KAEIDIEAISGR |
| 1301.7059 | 1301.6486 | −.0573 | −44 | 897 | 908 | KAEIDIEAISGR |
| 1320.6287 | 1320.6151 | −.0136 | −10 | 348 | 359 | NCILADEMGLGK |
| 1320.6287 | 1320.6151 | −.0136 | −10 | 348 | 359 | NCILADEMGLGK |
| 1332.6552 | 1332.6293 | −.0259 | −19 | 610 | 619 | KCCNHPYLIK |
| 1332.6552 | 1332.6293 | −.0259 | −19 | 610 | 619 | KCCNHPYLIK |
| 1741.7698 | 1741.7495 | −.0203 | −12 | 826 | 841 | RGAYGAIMEEE |
| | | | | | | DEGSK |
| 1803.8177 | 1803.739 | −.0787 | −44 | 1793 | 1808 | EEHDQSTQMSK |
| | | | | | | EGGLK |
| 1803.8177 | 1803.739 | −.0787 | −44 | 1793 | 1808 | EEHDQSTQMSK |
| | | | | | | EGGLK |
| 1838.9218 | 1838.9127 | −.0091 | −5 | 1163 | 1177 | YNTIRADPALCF |
| | | | | | | LER |
| 2396.1133 | 2395.948 | −.1653 | −69 | 1522 | 1542 | EEPQSSEEESM |
| | | | | | | SSVETR TLIK |

| | |
|---|---|
| mitotic kinesin-related protein [Homo sapiens] | gi\|15919888 |

Peptide Information

| | Obsrv. | | | Start | End | |
|---|---|---|---|---|---|---|
| Calc. Mass | Mass | ±da | ±ppm | Seq. | Seq. | Sequence |
| 832.4563 | 832.4887 | .0324 | 39 | 1461 | 1466 | KWLEEK |
| 895.493 | 895.4698 | −.0232 | −26 | 191 | 197 | MNLKPHR |
| 917.4839 | 917.4818 | −.0021 | −2 | 336 | 343 | HQSVAFTK |
| 932.52 | 932.4993 | −.0207 | −22 | 351 | 358 | SHSIFTIK |
| 932.52 | 932.4993 | −.0207 | −22 | 351 | 358 | SHSIFTIK |
| 933.5363 | 933.4963 | −.04 | −43 | 1552 | 1559 | RIISETSK |
| 948.5149 | 948.4807 | −.0342 | −36 | 118 | 126 | VFGPATTQK |
| 960.4996 | 960.5235 | .0239 | 25 | 903 | 910 | EEKAELNK |
| 982.4363 | 982.4796 | .0433 | 44 | 1282 | 1289 | EEDYADLK |
| 988.5058 | 988.5498 | .044 | 45 | 1571 | 1579 | RISSADPDK |
| 1033.5347 | 1033.5291 | −.0056 | −5 | 1371 | 1378 | EKIIEDMR |
| 1199.7246 | 1199.6718 | −.0528 | −44 | 576 | 585 | LLDLIEDLKK |
| 1229.71 | 1229.8047 | .0947 | 77 | 1116 | 1125 | EKETLIQQLK |
| 1258.6385 | 1258.5676 | −.0709 | −56 | 629 | 638 | EILEENAERR |
| 1301.677 | 1301.6486 | −.0284 | −22 | 811 | 821 | LICNETVEVPK |
| 1301.677 | 1301.6486 | −.0284 | −22 | 811 | 821 | LICNETVEVPK |
| 1315.729 | 1315.6655 | −.0635 | −48 | 1560 | 1570 | IETQIMDIKPK |
| 1320.6464 | 1320.6151 | −.0313 | −24 | 359 | 369 | ILQIEDSEMSR |
| 1320.6464 | 1320.6151 | −.0313 | −24 | 359 | 369 | ILQIEDSEMSR |
| 1487.8251 | 1487.7683 | −.0568 | −38 | 1560 | 1571 | IETQIMDIKPKR |
| 1505.7992 | 1505.7354 | −.0638 | −42 | 961 | 973 | IMKLSNEIETATR |
| 1505.7992 | 1505.7354 | −.0638 | −42 | 961 | 973 | IMKLSNEIETATR |
| 1740.7415 | 1740.7317 | −.0098 | −6 | 1658 | 1671 | SNEMEEDLVKCENK |
| 1741.9232 | 1741.7495 | −.1737 | −100 | 143 | 158 | GQSRLIFTYGLTNS |
| | | | | | | GK |
| 1838.9283 | 1838.9127 | −.0156 | −8 | 159 | 174 | TYTFQGTEENI |
| | | | | | | GILPR |

| | |
|---|---|
| unnamed protein product [Homo sapiens] | gi\|215415654 |

Protein Group

| | |
|---|---|
| unnamed protein product [Homo sapiens] | gi\|215415646 |
| unnamed protein product [Homo sapiens] | gi\|215415632 |
| unnamed protein product [Homo sapiens] | gi\|215415630 |
| unnamed protein product [Homo sapiens] | gi\|215415628 |
| unnamed protein product [Homo sapiens] | gi\|215415658 |

Peptide Information

| Calc. | Obsrv. | | | Start | End | |
|---|---|---|---|---|---|---|
| Mass | Mass | ±da | ±ppm | Seq. | Seq. | Sequence |
| 831.4359 | 831.4731 | .0372 | 45 | 189 | 195 | LAEYHAK |
| 1226.5437 | 1226.5537 | .01 | 8 | 1 | 10 | DEPPQSPW |
| | | | | | | DR |
| 1226.5437 | 1226.5537 | .01 | 8 | 1 | 10 | DEPPQSPW |
| | | | | | | DR |
| 1301.6484 | 1301.6486 | .0002 | 0 | 161 | 171 | THLAPYSD |
| | | | | | | ELR |
| 1301.6484 | 1301.6486 | .0002 | 0 | 161 | 171 | THLAPYSD |
| | | | | | | ELR |

| | |
|---|---|
| ciprofibrate bound protein p240 isoform | gi\|68164076 |
| PRIC320-2 [Homo sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 800.4876 | 800.43 | −.0576 | −72 | 1083 | 1090 | DLLIGAAK |
| 802.457 | 802.4542 | −.0028 | −3 | 713 | 718 | AEWLRK |
| 807.3843 | 807.409 | .0247 | 31 | 1929 | 1936 | NSDLGSSK |
| 854.5458 | 854.5139 | −.0319 | −37 | 1715 | 1722 | ILTGPVVR |
| 856.5363 | 856.5127 | −.0236 | −28 | 1731 | 1738 | RPKSGIAK |
| 887.4832 | 887.4777 | −.0055 | −6 | 465 | 471 | EIEDLLR |
| 903.4828 | 903.4889 | .0061 | 7 | 1375 | 1381 | DRVMINR |
| 917.4839 | 917.4818 | −.0021 | −2 | 1096 | 1102 | TDYHILR |
| 932.5121 | 932.4993 | −.0128 | −14 | 1153 | 1160 | GIILEEMK |
| 932.5121 | 932.4993 | −.0128 | −14 | 1153 | 1160 | GIILEEMK |
| 948.4931 | 948.4807 | −.0124 | −13 | 443 | 451 | AVLQSMSGR |
| 1012.5105 | 1012.5838 | .0733 | 72 | 406 | 413 | CHRIGQNK |
| 1191.5826 | 1191.6141 | .0315 | 26 | 814 | 823 | ADPALCFL |
| | | | | | | ER |
| 1198.7042 | 1198.6854 | −.0188 | −16 | 1185 | 1195 | TLIKSEPV |
| | | | | | | SPK |
| 1301.7059 | 1301.6486 | −.0573 | −44 | 543 | 554 | KAEIDIEAI |
| | | | | | | SGR |
| 1301.7059 | 1301.6486 | −.0573 | −44 | 543 | 554 | KAEIDIEAISGR |
| 1332.6552 | 1332.6293 | −.0259 | −19 | 256 | 265 | KCCNHPYLIK |
| 1332.6552 | 1332.6293 | −.0259 | −19 | 256 | 265 | KCCNHPYLIK |
| 1357.8058 | 1357.717 | −.0888 | −65 | 184 | 194 | LQVILKPMMLR |
| 1741.7698 | 1741.7495 | −.0203 | −12 | 472 | 487 | RGAYGAIMEEE |
| | | | | | | DEGSK |
| 1803.8177 | 1803.739 | −.0787 | −44 | 1439 | 1454 | EEHDQSTQMSK |
| | | | | | | EGGLK |
| 1803.8177 | 1803.739 | −.0787 | −44 | 1439 | 1454 | EEHDQSTQMSK |
| | | | | | | EGGLK |
| 1838.9218 | 1838.9127 | −.0091 | −5 | 809 | 823 | YNTIRADPALCF |
| | | | | | | LER |
| 2396.1133 | 2395.948 | −.1653 | −69 | 1168 | 1188 | EEPQSSEEESM |
| | | | | | | SSVETR TLIK |

parvalbumin [Homo sapiens]

Protein Group

| | |
|---|---|
| parvalbumin [Homo sapiens] | gi\|169158851 |
| | gi\|220897517 |

Peptide Information

| | Obsrv. | | | Start | End | |
|---|---|---|---|---|---|---|
| Calc. Mass | Mass | ±da | ±ppm | Seq. | Seq. | Sequence |
| 852.3953 | 852.4766 | .0813 | 95 | 83 | 90 | MLMAAGDK |
| 891.4781 | 891.4754 | −.0027 | −3 | 75 | 82 | DLSAKETK |
| 905.455 | 905.4752 | .0202 | 22 | 45 | 51 | VFHMLDK |
| 1033.5499 | 1033.5291 | −.0208 | −20 | 44 | 51 | KVFHMLDK |
| 1226.5756 | 1226.5537 | −.0219 | −18 | 80 | 90 | ETKMLMAAGDK |
| 1226.5756 | 1226.5537 | −.0219 | −18 | 80 | 90 | ETKMLMAAGDK |
| 1262.6296 | 1262.63 | .0004 | 0 | 1 | 11 | MTDLLNAEDIK |
| 1487.6497 | 1487.7683 | .1186 | 80 | 91 | 105 | DGDGKIGVDDPAD |
| | | | | | | VD |

| | |
|---|---|
| SH2/SH3 adaptor protein NCK-beta [Homo sapiens] | gi\|2921587 |

Peptide Information

| | | | | Start | End | |
|---|---|---|---|---|---|---|
| Calc. Mass | Obsrv. Mass | ±da | ±ppm | Seq. | Seq. | Sequence |
| 800.4485 | 800.43 | −.0185 | −23 | 42 | 48 | VRNAANR |
| 903.4948 | 903.4889 | −.0059 | −7 | 38 | 43 | TWWRVR |
| 932.4935 | 932.4993 | .0058 | 6 | 128 | 135 | EDELSLVK |
| 932.4935 | 932.4993 | .0058 | 6 | 128 | 135 | EDELSLVK |
| 960.5472 | 960.5235 | −.0237 | −25 | 74 | 82 | DTLGLGKTR |
| 1001.5877 | 1001.5252 | −.0625 | −62 | 2 | 10 | TEEVIVIAK |
| 1033.5022 | 1033.5291 | .0269 | 26 | 186 | 196 | GASLSNGQGSR |
| 1226.5695 | 1226.5537 | −.0158 | −13 | 294 | 303 | HQAQCALNER |
| 1226.5695 | 1226.5537 | −.0158 | −13 | 294 | 303 | HQAQCALNER |
| 1487.7853 | 1487.7683 | −.017 | −11 | 26 | 37 | VNERLWLLDDSK |
| 1719.896 | 1719.7832 | −.1128 | −66 | 336 | 349 | VQLVDNVYCIGQRR |
| 1752.8076 | 1752.7648 | −.0428 | −24 | 11 | 24 | WDYTAQQDQELDIK |

| | |
|---|---|
| Chain A, The Helix-Hinge-Helix | gi\|157831262 |
| Structural Motif In | |
| Human Apolipoprotein A-I Determined | |
| By Nmr Spectroscopy, 1 Structure | |

Protein Group

| | Obsrv. | | | Start | End | |
|---|---|---|---|---|---|---|
| Calc. Mass | Mass | ±da | ±ppm | Seq. | Seq. | Sequence |
| 1001.5261 | 1001.5252 | −.0009 | −1 | 37 | 46 | LEALKENGGA |
| 1301.6484 | 1301.6486 | .0002 | 0 | 20 | 30 | THLAPYSDELR |
| 1301.6484 | 1301.6486 | .0002 | 0 | 20 | 30 | THLAPYSDELR |

| | |
|---|---|
| RecName: Full = Protein FAM74A4 | gi\|74754292 |

Peptide Information

| | |
|---|---|
| Chain A, The Helix-Hinge-Helix | gi\|157831263 |
| Structural Motif In | |
| Human Apolipoprotein A-I Determined | |
| By Nmr | |
| Spectroscopy, 1 Structure | |

Peptide Information

| | Obsrv. | | | Start | End | |
|---|---|---|---|---|---|---|
| Calc. Mass | Mass | ±da | ±ppm | Seq. | Seq. | Sequence |
| 800.4009 | 800.43 | .0291 | 36 | 93 | 99 | RHGESSK |
| 831.4835 | 831.4731 | −.0104 | −13 | 71 | 76 | TWKQLR |
| 832.4675 | 832.4887 | .0212 | 25 | 57 | 62 | TWKELR |
| 903.4418 | 903.4889 | .0471 | 52 | 63 | 70 | NSETVPEK |
| 1104.5103 | 1104.5414 | .0311 | 28 | 77 | 85 | GCLQEDVQR |
| 1246.5481 | 1246.671 | .1229 | 99 | 7 | 18 | GCPGGDVETAQR |
| 1301.6696 | 1301.6486 | −.021 | −16 | 60 | 70 | ELRNSETVPEK |
| 1301.6696 | 1301.6486 | −.021 | −16 | 60 | 70 | ELRNSETVPEK |
| 1487.7384 | 1487.7683 | .0299 | 20 | 77 | 88 | GCLQEDVQRVQR |

| | |
|---|---|
| apolipoprotein A-1 A175P variant [Homo sapiens] | gi\|19569153 |

Peptide Information

| | Obsrv. | | | Start | End | |
|---|---|---|---|---|---|---|
| Calc. Mass | Mass | ±da | ±ppm | Seq. | Seq. | Sequence |
| 831.4359 | 831.4731 | .0372 | 45 | 35 | 41 | LAEYHAK |
| 1301.6484 | 1301.6486 | .0002 | 0 | 7 | 17 | THLAPYSDELR |
| 1301.6484 | 1301.6486 | .0002 | 0 | 7 | 17 | THLAPYSDELR |

| | |
|---|---|
| novel protein [Homo sapiens] | gi\|57209555 |

Peptide Information

| | Obsrv. | | | Start | End | |
|---|---|---|---|---|---|---|
| Calc. Mass | Mass | ±da | ±ppm | Seq. | Seq. | Sequence |
| 807.4471 | 807.409 | −.0381 | −47 | 90 | 96 | DLGLHPR |
| 887.5057 | 887.4777 | −.028 | −32 | 57 | 64 | ARGLDLSR |
| 903.4894 | 903.4889 | −.0005 | −1 | 139 | 146 | SVGEDKIR |
| 1246.675 | 1246.671 | −.004 | −3 | 47 | 58 | GLGSQTESLKAR |
| 1315.6787 | 1315.6655 | −.0132 | −10 | 67 | 77 | TCVVVAEERPR |
| 1740.7278 | 1740.7317 | .0039 | 2 | 33 | 46 | DTFCSYSVMELCTK |

| | |
|---|---|
| unnamed protein product [Homo sapiens] | gi\|7020094 |

Peptide Information

| Calc. Mass | Obsrv. | ±da | ±ppm | Start | End | Sequence |
|---|---|---|---|---|---|---|
| 814.4345 | 814.4791 | .0446 | 55 | 13 | 13 | YIEVYK |
| 832.4457 | 832.4887 | .043 | 52 | 28 | 29 | DCIRLR |
| 903.4319 | 903.4889 | .057 | 63 | 11 | 11 | FVSEEHR |
| 996.5625 | 996.6213 | .0588 | 59 | 23 | 24 | RYIELFR |
| 1191.5762 | 1191.614 | .0379 | 32 | 34 | 35 | AFMAAQKCHK |
| 1254.6113 | 1254.649 | .0377 | 30 | 4 | 49 | FSDPERVNYK |
| 1315.6965 | 1315.665 | −.031 | −24 | 24 | 25 | STAAEVQQVLNR |
| 1320.6465 | 1320.615 | −.0314 | −24 | 5 | 68 | MELIDDNTVVR |
| 1320.6465 | 1320.615 | −.0314 | −24 | 5 | 68 | MELIDDNTVVR |
| 1646.8021 | 1646.841 | .0397 | 24 | 2 | 15 | TEYLNFEKSSSVS |

parvalbumin [Homo sapiens]

Protein Group

| | |
|---|---|
| parvalbumin [Homo sapiens] | gi\|169158850 |
| | gi\|220897515 |

Peptide Information

| | Obsrv. | | | Start | End | |
|---|---|---|---|---|---|---|
| Calc. Mass | Mass | ±da | ±ppm | Seq. | Seq. | Sequence |
| 852.3953 | 852.4766 | .0813 | 95 | 53 | 60 | MLMAAGDK |
| 891.4781 | 891.4754 | −.0027 | −3 | 45 | 52 | DLSAKETK |
| 905.455 | 905.4752 | .0202 | 22 | 15 | 21 | VFHMLDK |
| 1033.5499 | 1033.5291 | −.0208 | −20 | 14 | 21 | KVFHMLDK |
| 1226.5756 | 1226.5537 | −.0219 | −18 | 50 | 60 | ETKMLMAAGDK |
| 1226.5756 | 1226.5537 | −.0219 | −18 | 50 | 60 | ETKMLMAAGDK |
| 1838.8655 | 1838.9127 | .0472 | 26 | 61 | 78 | DGDGKIGVDEFSTL |
| | | | | | | VAES |

| | |
|---|---|
| prefoldin subunit 6 [Homo sapiens] | gi\|123270303 |

Protein Group

| | |
|---|---|
| prefoldin subunit 6 [Homo sapiens] | gi\|168985736 |

Peptide Information

| | | | | Start | End | |
|---|---|---|---|---|---|---|
| Calc. Mass | Obsrv. Mass | ±da | ±ppm | Seq. | Seq. | Sequence |
| 802.4305 | 802.4542 | .0237 | 30 | 9 | 15 | LQGEVEK |
| 807.4359 | 807.409 | −.0269 | −33 | 16 | 21 | YQQLQK |
| 809.3934 | 809.3939 | .0005 | 1 | 26 | 32 | SMSGRQK |
| 832.4597 | 832.4887 | .029 | 35 | 1 | 7 | MAELIQK |
| 960.5546 | 960.5235 | −.0311 | −32 | 1 | 8 | MAELIQKK |
| 1258.675 | 1258.5676 | −.1074 | −85 | 67 | 78 | QELGEARATVGK |

| | |
|---|---|
| Chain A, Crystal Structure OfHuman | gi\|110590538 |
| Pyrroline-5-Carboxylate Synthetase | |

Protein Group

| | |
|---|---|
| Chain B, Crystal Structure Of | gi\|110590539 |
| Human | |
| Pyrroline-5-Carboxylate Synthetase | |

Peptide Information

| | Obsrv. | | | Start | End | |
|---|---|---|---|---|---|---|
| Calc. Mass | Mass | ±da | ±ppm | Seq. | Seq. | Sequence |
| 995.636 | 995.6278 | −.0082 | −8 | 219 | 226 | XIDLIIPR |

| | |
|---|---|
| ribosomal protein L39 variant | gi\|62087734 |
| [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 995.5533 | 995.6278 | .0745 | 75 | 1 | 8 | RVPHFSPR |

### 2—Fraction III

The sequence of these 8 new found proteins and their function are being investigated.

| | | | |
|---|---|---|---|
| | 29/B2 | | Instr./Gel |
| | | | Origin |
| | [1] Sample Project | | Instrument |
| | 20110503 | | Sample |
| | | | Name |
| Protein Name- | KH3 (SEQ ID NO. KH3) | Accession No. | |

| | |
|---|---|
| unnamed protein product [Homo sapiens]- | gi\|40044478 |
| Peptide Information | |

| | Obsrv. | | | Start | End | |
|---|---|---|---|---|---|---|
| Calc. Mass | Mass | ±da | ±ppm | Seq. | Seq. | Sequence |
| 841.4124 | 841.3352 | −.0772 | −92 | 36 | 42 | GTEYLMK |
| 857.4073 | 857.3639 | −.0434 | −51 | 36 | 42 | GTEYLMK |
| 857.4073 | 857.3639 | −.0434 | −51 | 36 | 42 | GTEYLMK |
| 1017.5225 | 1017.4292 | −.0933 | −92 | 62 | 71 | GAQKTGGGWR |
| 1097.566 | 1097.4915 | −.0745 | −68 | 36 | 44 | GTEYLMK |
| | | | | | | QK |
| 1235.7107 | 1235.683 | −.0277 | −22 | 51 | 61 | GEELLLGV |
| | | | | | | HLR |

| | |
|---|---|
| voltage-dependent anion channel 3, isoform | gi\|119583621 |
| CRA_a [Homo sapiens] | |

Peptide Information

| Calc. Mass | Obsrv. Mass | ±da | ±ppm | Start Seq. | End Seq. | Sequence |
|---|---|---|---|---|---|---|
| 815.3604 | 815.3253 | −.0351 | −43 | 1 | 7 | MSFDTAK |
| 817.3951 | 817.3161 | −.079 | −97 | 113 | 120 | NFSAGGHK |
| 857.3644 | 857.3639 | −.0005 | −1 | 71 | 76 | YMLDCR |
| 857.3644 | 857.3639 | −.0005 | −1 | 71 | 76 | YMLDCR |
| 1014.4924 | 1014.4064 | −.086 | −85 | 1 | 9 | MSFDTAKSK |
| 1030.4874 | 1030.429 | −.0584 | −57 | 1 | 9 | MSFDTAKSK |

| | |
|---|---|
| cysteine-rich protein 2 variant [Homo sapiens] | gi\|62898063 |

Peptide Information

| Calc. | Obsrv. | | | Start | End | |
|---|---|---|---|---|---|---|
| Mass | Mass | ±da | ±ppm | Seq. | Seq. | Sequence |
| 807.3488 | 807.3163 | −.0325 | −40 | 5 | 10 | CPKCDK |
| 809.3392 | 809.3123 | −.0269 | −33 | 153 | 158 | CERCGK |
| 821.4008 | 821.3269 | −.0739 | −90 | 1 | 7 | MASKCPK |
| 837.3957 | 837.3484 | −.0473 | −56 | 1 | 7 | MASKCPK |
| 857.4403 | 857.3639 | −.0764 | −89 | 11 | 17 | TVYFAEK |
| 857.4403 | 857.3639 | −.0764 | −89 | 11 | 17 | TVYFAEK |
| 1012.4703 | 1012.4331 | −.0372 | −37 | 28 | 34 | FCLKCER |

| | |
|---|---|
| D53 [Homo sapiens] | gi\|1469924 |

Peptide Information

| Calc. | Obsrv. | | | Start | End | |
|---|---|---|---|---|---|---|
| Mass | Mass | ±da | ±ppm | Seq. | Seq. | Sequence |
| 807.3189 | 807.3163 | −.0026 | −3 | 55 | 60 | EEELQC |
| 825.3672 | 825.3186 | −.0486 | −59 | 1 | 6 | CSFQQR |
| 1036.4615 | 1036.4291 | −.0324 | −31 | 53 | 60 | TKEEELQC |

| | |
|---|---|
| cysteine-rich protein 2 [Homo sapiens] | gi\|4503049 |

Protein Group

| | |
|---|---|
| Cysteine-rich protein 2 [Homo sapiens] | gi\|12653331 |
| Cysteine-rich protein 2 [Homo sapiens] | gi\|21706672 |
| Cysteine-rich protein 2 [Homo sapiens] | gi\|118763570 |
| Cysteine-rich protein 2 [Homo sapiens] | gi\|12804957 |
| LIM domain protein ESP1/CRP1 | gi\|1587284 |
| LIM domain protein ESP1/CRP1 | |
| RecName: Full = Cysteine-rich protein 2; | gi\|1706133 |
| Short = CRP-2; AltName: Full = Protein ESP1 | |
| cystein-rich protein 2 [Homo sapiens] | gi\|1215803 |
| cysteine-rich protein 2 [Homo sapiens] | gi\|1399028 |
| cysteine-rich protein 2 [Homo sapiens] | gi\|54696684 |
| unnamed protein product [Homo sapiens] | gi\|189055126 |

Peptide Information

| Calc. | Obsrv. | | | Start | End | |
|---|---|---|---|---|---|---|
| Mass | Mass | ±da | ±ppm | Seq. | Seq. | Sequence |
| 807.3488 | 807.3163 | −.0325 | −40 | 5 | 10 | CPKCDK |
| 809.3392 | 809.3123 | −.0269 | −33 | 153 | 158 | CERCGK |
| 821.4008 | 821.3269 | −.0739 | −90 | 1 | 7 | MASKCPK |
| 837.3957 | 837.3484 | −.0473 | −56 | 1 | 7 | MASKCPK |
| 857.4403 | 857.3639 | −.0764 | −89 | 11 | 17 | TVYFAEK |
| 857.4403 | 857.3639 | −.0764 | −89 | 11 | 17 | TVYFAEK |
| 1012.4703 | 1012.4331 | −.0372 | −37 | 28 | 34 | FCLKCER |

| | |
|---|---|
| cysteine-rich protein 2 [Homo sapiens] | gi\|119602330 |

Peptide Information

| Calc. Mass | Obsrv. | ±da | ±ppm | Start | End | Sequence |
|---|---|---|---|---|---|---|
| 807.3 | 807.3163 | −.0325 | — | 5 | 10 | CPKCDK |
| 821.4 | 821.3269 | −.0739 | — | 1 | 7 | MASKCPK |
| 837.3 | 837.3484 | −.0473 | — | 1 | 7 | MASKCPK |
| 857.4 | 857.3639 | −.0764 | — | 1 | 17 | TVYFAEK |
| 857.4 | 857.3639 | −.0764 | — | 1 | 17 | TVYFAEK |
| 1012.4703 | 1012.433 | −.0372 | — | 2 | 34 | FCLKCER |

| | |
|---|---|
| PREDICTED: hypothetical protein | gi\|310120478 |
| LOC100507339 [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 808.3406 | 808.317 | −.0236 | −29 | 113 | 118 | WNGMER |
| 837.3593 | 837.3484 | −.0109 | −13 | 165 | 171 | VECNGMK |
| 840.392 | 840.3284 | −.0636 | −76 | 106 | 112 | VEYNGMK |
| 895.3727 | 895.3547 | −.018 | −20 | 172 | 178 | WSGMERN |
| 895.3727 | 895.3547 | −.018 | −20 | 172 | 178 | WSGMERN |

| | |
|---|---|
| unknown [Homo sapiens] | gi\|62420312 |

Peptide Information

| Calc. | Obsrv. | | | Start | End | |
|---|---|---|---|---|---|---|
| Mass | Mass | ±da | ±ppm | Seq. | Seq. | Sequence |
| 822.3992 | 822.3179 | −.0813 | −99 | 5 | 10 | EEFIER |
| 842.4553 | 842.3948 | −.0605 | −72 | 58 | 64 | NMALHLK |
| 842.4553 | 842.3948 | −.0605 | −72 | 58 | 64 | NMALHLK |
| 1036.531 | 1036.4291 | −.1019 | −98 | 11 | 19 | IQGLDFDTK |

| | |
|---|---|
| zinc finger protein 726 [Homo | gi\|299890850 |
| sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 804.3708 | 804.3198 | −.051 | −63 | 61 | 66 | EPWNMK |
| 820.3658 | 820.3197 | −.0461 | −56 | 61 | 66 | EPWNMK |
| 1038.5037 | 1038.4131 | −.0906 | −87 | 31 | 38 | NVMLENYR |
| 1077.5034 | 1077.4087 | −.0947 | −88 | 59 | 66 | EKEPWNMK |

| | |
|---|---|
| polyglutamine binding protein 1, | gi\|119571120 |
| isoform CRA_a [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 804.3846 | 804.3198 | −.0648 | −81 | 191 | 197 | ADREEGK |
| 811.3251 | 811.3227 | −.0024 | −3 | 1 | 7 | MSDETGR |
| 814.3802 | 814.3175 | −.0627 | −77 | 157 | 163 | GHDKSDR |
| 814.3802 | 814.3175 | −.0627 | −77 | 133 | 139 | SDRGHDK |
| 824.3897 | 824.3263 | −.0634 | −77 | 187 | 193 | GYDKADR |
| 848.4148 | 848.3552 | −.0596 | −70 | 235 | 241 | EEAFPQK |
| 848.4148 | 848.3552 | −.0596 | −70 | 235 | 241 | EEAFPQK |
| 852.421 | 852.3447 | −.0763 | −90 | 168 | 174 | GYDKVDR |
| | 852.3447 | | | | | |

| | |
|---|---|
| Chain A, Solution Structure Of The | gi\|159163957 |
| Lim Domain Of Human Cysteine- | |
| Rich Protein 2 | |

Peptide Information

| Calc. | Obsrv. | | | Start | End | |
|---|---|---|---|---|---|---|
| Mass | Mass | ±da | ±ppm | Seq. | Seq. | Sequence |
| 807.3488 | 807.3163 | −.0325 | −40 | 12 | 17 | CPKCDK |
| 857.4403 | 857.3639 | −.0764 | −89 | 18 | 24 | TVYFAEK |
| 857.4403 | 857.3639 | −.0764 | −89 | 18 | 24 | TVYFAEK |
| 1012.4703 | 1012.4331 | −.0372 | −37 | 35 | 41 | FCLKCER |

| | |
|---|---|
| similar to RIKEN cDNA A430101B06 | gi\|119582663 |
| gene, isoform CRA_a [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 800.3533 | 800.3335 | −.0198 | −25 | 53 | 60 | DPPDGSGR |
| 800.405 | 800.3335 | −.0715 | −89 | 47 | 52 | HLPDYR |
| 829.3574 | 829.297 | −.0604 | −73 | 29 | 35 | SYSESEK |
| 829.3574 | 829.297 | −.0604 | −73 | 29 | 35 | SYSESEK |

| | |
|---|---|
| ZNF726 protein [Homo sapiens] | gi\|71052071 |

Peptide Information

| Calc. | Obsrv. | | | Start | End | |
|---|---|---|---|---|---|---|
| Mass | Mass | ±da | ±ppm | Seq. | Seq. | Sequence |
| 804.3708 | 804.3198 | −.051 | −63 | 61 | 66 | EPWNMK |
| 820.3658 | 820.3197 | −.0461 | −56 | 61 | 66 | EPWNMK |
| 1038.5037 | 1038.4131 | −.0906 | −87 | 31 | 38 | NVMLENYR |
| 1077.5034 | 1077.4087 | −.0947 | −88 | 59 | 66 | EKEPWNMK |

| | |
|---|---|
| T-cell receptor delta chain [Homo | gi\|2239988 |
| sapiens] | |

Peptide Information

| | Obsrv. | | | | | |
|---|---|---|---|---|---|---|
| Calc. Mass | Mass | ±da | ±ppm | Start Seq. | End Seq. | Sequence |
| 817.3727 | 817.3161 | −.0566 | −69 | 15 | 21 | GSFYTDK |
| 1031.5044 | 1031.4188 | −.0856 | −83 | 15 | 23 | GSFYTDKLT |

| | |
|---|---|
| CDKN2AIPNL protein [Homo sapiens] | gi\|17390191 |

Protein Group

| | |
|---|---|
| similar to RIKEN cDNA A430101B06 gene, isoform | gi\|119582664 |
| CRA_b [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 800.3533 | 800.3335 | −.0198 | −25 | 53 | 60 | DPPDGSGR |
| 800.405 | 800.3335 | −.0715 | −89 | 47 | 52 | HLPDYR |
| 829.3574 | 829.297 | −.0604 | −73 | 29 | 35 | SYSESEK |
| 829.3574 | 829.297 | −.0604 | −73 | 29 | 35 | SYSESEK |

| | |
|---|---|
| unnamed protein product [Homo sapiens] | gi\|21336226 |

Protein Group

| | |
|---|---|
| HSPC072 [Homo sapiens] | gi\|6841382 |

Peptide Information

| | Obsrv. | | | | | |
|---|---|---|---|---|---|---|
| Calc. Mass | Mass | ±da | ±ppm | Start Seq. | End Seq. | Sequence |
| 838.3909 | 838.3401 | −.0508 | −61 | 2 | 8 | MMTATQR |
| 1017.4162 | 1017.4292 | .013 | 13 | 1 | 8 | MMMTATQR |
| 1097.519 | 1097.4915 | −.0275 | −25 | 2 | 10 | MMTATQRAR |

| | |
|---|---|
| RecName: Full = Putative RNA-binding | gi\|1710620 |
| protein 3; | |
| AltName: Full = RNA-binding motif protein | |
| 3; AltName: | |
| Full = RNPL | |

Protein Group

| | |
|---|---|
| RNA binding motif (RNP1, RRM) protein | gi\|13905080 |
| 3 [Homo sapiens] | |
| RNA binding motif (RNP1, RRM) protein | gi\|119571153 |
| 3, isoform CRA_c [Homo sapiens] | |
| RNA binding motif (RNP1, RRM) protein | gi\|119571152 |
| 3, isoform CRA_c [Homo sapiens] | |
| RNPL [Homo sapiens] | gi\|881954 |
| putative RNA-binding protein 3 [Homo sapiens] | gi\|5803137 |
| unnamed protein product [Homo sapiens] | gi\|257297086 |

Peptide Information

| Calc. | Obsrv. | | | Start | End | |
|---|---|---|---|---|---|---|
| Mass | Mass | ±da | ±ppm | Seq. | Seq. | Sequence |
| 804.3958 | 804.3198 | −.076 | −94 | 40 | 45 | DRETQR |
| 809.3536 | 809.3123 | −.0413 | −51 | 139 | 145 | NQGGYDR |
| 815.3907 | 815.3253 | −.0654 | −80 | 91 | 99 | GGGFGAHGR |
| 816.3635 | 816.3242 | −.0393 | −48 | 146 | 152 | YSGGNYR |

| | |
|---|---|
| APAF1 protein [Homo sapiens] | gi\|6687822 |

Peptide Information

| | | | | | End | |
|---|---|---|---|---|---|---|
| Calc. Mass | Obsrv. Mass | ±da | ±ppm | Start Seq. | Seq. | Sequence |
| 835.4091 | 835.3354 | −.0737 | −88 | 8 | 14 | SKVADCR |
| 1014.4785 | 1014.4064 | −.0721 | −71 | 10 | 18 | VADCRGHLS |

| | |
|---|---|
| immunoglobulin heavy chain variable region | gi\|157058627 |
| [Homo sapiens] | |

Peptide Information

| | Obsrv. | | | | | |
|---|---|---|---|---|---|---|
| Calc. Mass | Mass | ±da | ±ppm | Start Seq. | End Seq. | Sequence |
| 845.4039 | 845.3217 | −.0822 | −97 | 59 | 65 | YYADSVK |
| 1017.5265 | 1017.4292 | −.0973 | −96 | 31 | 38 | SYGIHWVR |
| 1053.5323 | 1053.4489 | −.0834 | −79 | 68 | 76 | FTVSRDNSK |

| | |
|---|---|
| hCG22885, isoform CRA_a [Homo sapiens] | gi\|119602066 |

Peptide Information

| | | | | Start | End | |
|---|---|---|---|---|---|---|
| Calc. Mass | Obsrv. Mass | ±da | ±ppm | Seq. | Seq. | Sequence |
| 842.4553 | 842.3948 | −.0605 | −72 | 1 | 8 | MPAAALPR |
| 842.4553 | 842.3948 | −.0605 | −72 | 1 | 8 | MPAAALPR |
| 1053.5337 | 1053.4489 | −.0848 | −80 | 35 | 43 | AGQKGYHHR |

| | | | |
|---|---|---|---|
| | 30/B3 | | Instr./Gel |
| | | | Origin |
| | [1] Sample Project | | Instrument |
| | 20110503 | | Sample |
| | | | Name |
| Protein Name- | KH4 (SEQ ID NO. KH4) | Accession No. | |

| | |
|---|---|
| vinculin, isoform CRA_a [Homo sapiens] | gi\|119574932 |

Peptide Information

| | Obsrv. | | | Start | End | |
|---|---|---|---|---|---|---|
| Calc. Mass | Mass | ±da | ±ppm | Seq. | Seq. | Sequence |
| 802.4417 | 802.3699 | −.0718 | −89 | 574 | 582 | AAAVGTANK |
| 803.4443 | 803.3769 | −.0674 | −84 | 873 | 879 | ALIQCAK |
| 804.4461 | 804.3667 | −.0794 | −99 | 196 | 203 | ALASIDSK |
| 807.3777 | 807.4016 | .0239 | 30 | 898 | 903 | QCTDKR |
| 815.4733 | 815.4049 | −.0684 | −84 | 337 | 344 | GALAEARK |
| 817.4236 | 817.3787 | −.0449 | −55 | 90 | 97 | NLGPGMTK |
| 818.4254 | 818.3765 | −.0489 | −60 | 967 | 974 | EAEAASIK |
| 820.4047 | 820.37 | −.0347 | −42 | 712 | 719 | AASDELSK |
| 824.4625 | 824.4018 | −.0607 | −74 | 416 | 423 | AAVHLEGK |
| 833.4185 | 833.4044 | −.0141 | −17 | 236 | 243 | VGELCAGK |
| 850.4087 | 850.3828 | −.0259 | −30 | 189 | 195 | DTEAMKR |
| 866.4036 | 866.402 | −.0016 | −2 | 189 | 195 | DTEAMKR |
| 976.5244 | 976.5515 | .0271 | 28 | 246 | 253 | REILGTCK |
| 1037.599 | 1037.5863 | −.0127 | −12 | 138 | 146 | IFVTTKNSK |
| 1229.7174 | 1229.837 | .1196 | 97 | 127 | 137 | ELLPVLISAMK |
| 1229.7174 | 1229.837 | .1196 | 97 | 127 | 137 | ELLPVLISAMK |
| 1235.6487 | 1235.6796 | .0309 | 25 | 116 | 126 | VMLVNSMN |
| | | | | | | TVK |
| 1642.015 | 1641.8651 | −.1499 | −91 | 915 | 929 | IPTISTQLKIL |
| | | | | | | STVK |

| | |
|---|---|
| proopiomelanocortin, POMC = long allele product | gi\|998478 |
| [human, pituitary glands, Peptide Partial, 33 aa] | |

Peptide Information

| | | | | Start | End | |
|---|---|---|---|---|---|---|
| Calc. Mass | Obsrv. Mass | ±da | ±ppm | Seq. | Seq. | Sequence |
| 836.4162 | 836.3588 | −.0574 | −69 | 10 | 15 | WDRFGR |
| 1037.535 | 1037.5863 | .0513 | 49 | 2 | 9 | KYVMGHFR |
| 1366.6473 | 1366.6088 | −.0385 | −28 | 3 | 12 | YVMGHFRWDR |
| 1540.6947 | 1540.6804 | −.0143 | −9 | 17 | 33 | NSSSSGSSGSSG [symbol] |
| | | | | | | GQKR |

| | |
|---|---|
| unnamed protein product [Homo sapiens] | gi\|40033811 |

[symbol]

indicates data missing or illegible when filed

Protein Group

| | |
|---|---|
| unnamed protein product [Homo sapiens] | gi\|10433748 |

Peptide Information

| | | | | Start | End | |
|---|---|---|---|---|---|---|
| Calc. Mass | Obsrv. Mass | ±da | ±ppm | Seq. | Seq. | Sequence |
| 803.3893 | 803.3769 | −.0124 | −15 | 339 | 344 | LEEEQR |
| 804.3846 | 804.3667 | −.0179 | −22 | 332 | 337 | EEENRK |
| 810.4104 | 810.3868 | −.0236 | −29 | 402 | 408 | KSGYNNK |
| 810.4104 | 810.3868 | −.0236 | −29 | 402 | 408 | KSGYNNK |
| 816.4573 | 816.3807 | −.0766 | −94 | 484 | 490 | NENLAKK |
| 818.4002 | 818.3765 | −.0237 | −29 | 352 | 357 | QKEEER |
| 825.3988 | 825.3849 | −.0139 | −17 | 363 | 368 | EYEEKK |
| 826.4457 | 826.3818 | −.0639 | −77 | 296 | 302 | AFVAYQK |
| 864.4131 | 864.4175 | .0044 | 5 | 492 | 498 | CSEELVK |
| 907.3639 | 907.4031 | .0392 | 43 | 27 | 34 | EDNESDAK |
| 912.4131 | 912.3611 | −.052 | −57 | 232 | 238 | MNDELYK |
| 919.4367 | 919.4315 | −.0052 | −6 | 2 | 9 | DDDDAKLK |
| 976.4945 | 976.5515 | .057 | 58 | 510 | 518 | TELGNSDLK |
| 1017.5098 | 1017.5444 | .0346 | 34 | 10 | 18 | AEIEAELDK |
| 1540.6948 | 1540.6804 | −.0144 | −9 | 227 | 238 | QEQDKMNDELYK |

| | |
|---|---|
| zinc finger protein 770 [Homo sapiens] | gi\|46094072 |

Protein Group

| | |
|---|---|
| RecName: Full = Zinc finger protein 770 | gi\|74757996 |
| Zinc finger protein 770 [Homo sapiens] | gi\|47938791 |
| hypothetical protein FLJ20582 [Homo sapiens] | gi\|119612730 |
| unnamed protein product [Homo sapiens] | gi\|158256312 |

Peptide Information

| Calc. Mass | Obsrv. Mass | ±da | ±ppm | Start Seq. | End Seq. | Sequence |
|---|---|---|---|---|---|---|
| 807.4723 | 807.4016 | −.0707 | −88 | 224 | 230 | GFKIQSK |
| 808.4312 | 808.3741 | −.0571 | −71 | 633 | 639 | SFRSPSK |
| 816.4573 | 816.3807 | −.0766 | −94 | 636 | 642 | SPSKLER |
| 823.3549 | 823.3546 | −.0003 | 0 | 83 | 88 | CSICQR |
| 823.3549 | 823.3546 | −.0003 | 0 | 83 | 88 | CSICQR |
| 824.3971 | 824.4018 | .0047 | 6 | 168 | 174 | MFPSQSK |
| 830.4155 | 830.4167 | .0012 | 1 | 328 | 333 | RSYNYK |
| 833.4437 | 833.4044 | −.0393 | −47 | 684 | 691 | VVALDSVM |
| 835.3978 | 835.3892 | −.0086 | −10 | 2 | 8 | MAENNLK |
| 845.4152 | 845.4222 | .007 | 8 | 35 | 41 | HFETPSK |
| 910.4893 | 910.4322 | −.0571 | −63 | 671 | 677 | HQLTHFK |
| 919.522 | 919.4315 | −.0905 | −98 | 234 | 240 | HKQIHTR |
| 998.4876 | 998.4788 | −.0088 | −9 | 139 | 146 | WALHPCSK |
| 998.4876 | 998.4788 | −.0088 | −9 | 139 | 146 | WALHPCSK |
| 1235.5773 | 1235.6796 | .1023 | 83 | 83 | 91 | CSICQRHFK |

| | |
|---|---|
| FGF-5 protein 1 [Homo sapiens] | gi\|182541 |

Peptide Information

| Calc. Mass | Obsrv. Mass | ±da | ±ppm | Start Seq. | End Seq. | Sequence |
|---|---|---|---|---|---|---|
| 807.4294 | 807.4016 | −.0278 | −34 | 29 | 35 | MHLGPPR |
| 823.4243 | 823.3546 | −.0697 | −85 | 29 | 35 | MHLGPPR |
| 823.4243 | 823.3546 | −.0697 | −85 | 29 | 35 | MHLGPPR |
| 848.4261 | 848.419 | −.0071 | −8 | 22 | 28 | AQNQPYK |
| 848.4261 | 848.419 | −.0071 | −8 | 22 | 28 | AQNQPYK |
| 864.3628 | 864.4175 | .0547 | 63 | 5 | 12 | CGEAGQSR |
| 1540.7034 | 1540.6804 | −.023 | −15 | 5 | 18 | CGEAGQSRG |
| | | | | | | TQPHR |

Protein Group

| | |
|---|---|
| Short = VACVase; Short = Valacyclovirase; | |
| Full = Biphenyl hydrolase-like protein; | |
| Full = Biphenyl hydrolase-related protein; | |
| Short = Bph-rp; | |
| antigen; Short = MCNAA; Flags: | |
| biphenyl hydrolase-like (serine | gi\|2895059 |
| epithelial mucin-associated antigen) | |
| biphenyl hydrolase-like protein [Homo sapiens] | gi\|3991880 |

Peptide Information

| | Obsrv. | | | Start | End | |
|---|---|---|---|---|---|---|
| Calc. Mass | Mass | ±da | ±ppm | Seq. | Seq. | Sequence |
| 807.3917 | 807.4016 | .0099 | 12 | 120 | 126 | DAVDLMK |
| 818.4553 | 818.3765 | −.0788 | −96 | 1 | 8 | MVAVLGGR |
| 835.4196 | 835.3892 | −.0304 | −36 | 285 | 291 | LAEDFLQ |
| 907.4672 | 907.4031 | −.0641 | −71 | 152 | 158 | YPSYIHK |
| 940.4921 | 940.4529 | −.0392 | −42 | 264 | 271 | LHLMPEGK |
| 1141.5498 | 1141.5339 | −.0159 | −14 | 98 | 107 | GYGHSRPPDR |
| 1227.7355 | 1227.8146 | .0791 | 64 | 1 | 12 | MVAVLGGRGVLR |

| | |
|---|---|
| hCG2045292 [Homo sapiens] | gi\|119593419 |

Peptide Information

| Calc. Mass | Obsrv. Mass | ±da | ±ppm | Start Seq. | End Seq. | Sequence |
|---|---|---|---|---|---|---|
| 807.4029 | 807.4016 | −.0013 | −2 | 4 | 9 | IEDMKR |
| 847.4407 | 847.4076 | −.0331 | −39 | 11 | 17 | ELTEEVK |
| 850.4338 | 850.3828 | −.051 | −60 | 2 | 8 | SKIEDMK |
| 866.4288 | 866.402 | −.0268 | −31 | 2 | 8 | SKIEDMK |
| 998.5305 | 998.4788 | −.0517 | −52 | 58 | 65 | LFSFEISR |
| 998.5305 | 998.4788 | −.0517 | −52 | 58 | 65 | LFSFEISR |
| 1017.4847 | 1017.5444 | .0597 | 59 | 18 | 26 | KGDQEEPSK |

| | |
|---|---|
| Chain A, Solution Structure Of Human Mekk3 Pb1 | gi\|149243322 |
| Domain | |

Protein Group

| | |
|---|---|
| Chain A, Solution Sturcture Of Human Mekk3 Pb1 | gi\|152149163 |
| Domain Cis Isomer | |

Peptide Information

| Calc. Mass | Obsrv. Mass | ±da | ±ppm | Start Seq. | End Seq. | Sequence |
|---|---|---|---|---|---|---|
| 815.4621 | 815.4049 | −.0572 | −70 | 63 | 69 | AIDILDR |
| 845.4839 | 845.4222 | −.0617 | −73 | 2 | 8 | QSDVRIK |
| 847.3792 | 847.4076 | .0284 | 34 | 56 | 62 | NQDDLDK |
| 919.4156 | 919.4315 | .0159 | 17 | 26 | 32 | YEDVEHK |
| 976.5244 | 976.5515 | .0271 | 28 | 1 | 8 | MQSDVRIK |

| | |
|---|---|
| KIAA1893 protein [Homo sapiens] | gi\|15620845 |

Peptide Information

| | Obsrv. | | | Start | End | |
|---|---|---|---|---|---|---|
| Calc. Mass | Mass | ±da | ±ppm | Seq. | Seq. | Sequence |
| 801.4213 | 801.3785 | −.0428 | −53 | 624 | 631 | ADPRASGK |
| 802.4305 | 802.3699 | −.0606 | −76 | 275 | 282 | VAPTSAEK |
| | 802.3699 | | | | | |
| 817.405 | 817.3787 | −.0263 | −32 | 481 | 488 | VNPESSGK |
| 818.4254 | 818.3765 | −.0489 | −60 | 704 | 711 | TESPSLGK |
| 823.4308 | 823.3546 | −.0762 | −93 | 227 | 233 | TYTVSPR |
| 823.4308 | 823.3546 | −.0762 | −93 | 227 | 233 | TYTVSPR |
| 830.4982 | 830.4167 | −.0815 | −98 | 283 | 290 | VDLVLSGK |
| 856.4774 | 856.5166 | .0392 | 46 | 187 | 194 | AEPEILGK |
| 858.4025 | 858.3828 | −.0197 | −23 | 155 | 161 | MDFKSSK |
| 858.4025 | 858.3828 | −.0197 | −23 | 155 | 161 | MDFKSSK |
| 866.4077 | 866.402 | −.0057 | −7 | 179 | 186 | ADPMFTGK |
| 912.4421 | 912.3611 | −.081 | −89 | 267 | 274 | EHPVSSEK |
| 931.4479 | 931.4319 | −.016 | −17 | 466 | 474 | EDPISAGSR |
| 1017.5211 | 1017.5444 | .0233 | 23 | 663 | 671 | KETPQASEK |
| 1598.7778 | 1598.7869 | .0091 | 6 | 195 | 209 | GDPVAPGRMDPMTVR |

| | |
|---|---|
| unnamed protein product [Homo sapiens] | gi\|193785261 |

Peptide Information

| | Obsrv. | | | Start | End | |
|---|---|---|---|---|---|---|
| Calc. Mass | Mass | ±da | ±ppm | Seq. | Seq. | Sequence |
| 802.4417 | 802.3699 | −.0718 | −89 | 574 | 582 | AAAVGTANK |
| 803.4443 | 803.3769 | −.0674 | −84 | 873 | 879 | ALIQCAK |
| 804.4461 | 804.3667 | −.0794 | −99 | 196 | 203 | ALASIDSK |
| 807.3777 | 807.4016 | .0239 | 30 | 898 | 903 | QCTDKR |
| 815.4733 | 815.4049 | −.0684 | −84 | 337 | 344 | GALAEARK |
| 817.4236 | 817.3787 | −.0449 | −55 | 90 | 97 | NLGPGMTK |
| 820.4047 | 820.37 | −.0347 | −42 | 712 | 719 | AASDELSK |
| 824.4625 | 824.4018 | −.0607 | −74 | 416 | 423 | AAVHLEGK |
| 833.4185 | 833.4044 | −.0141 | −17 | 236 | 243 | VGELCAGK |
| 850.4087 | 850.3828 | −.0259 | −30 | 189 | 195 | DTEAMKR |
| 866.4036 | 866.402 | −.0016 | −2 | 189 | 195 | DTEAMKR |
| 976.5244 | 976.5515 | .0271 | 28 | 246 | 253 | REILGTCK |
| 1037.599 | 1037.5863 | −.0127 | −12 | 138 | 146 | IFVTTKNSK |
| 1229.7174 | 1229.837 | .1196 | 97 | 127 | 137 | ELLPVLISAMK |
| 1229.7174 | 1229.837 | .1196 | 97 | 127 | 137 | ELLPVLISAMK |
| 1235.6487 | 1235.6796 | .0309 | 25 | 116 | 126 | VMLVNSMN |
| | | | | | | TVK |
| 1642.015 | 1641.8651 | −.1499 | −91 | 915 | 929 | IPTISTQLKIL |
| | | | | | | STVK |

| | |
|---|---|
| unnamed protein product [Homo sapiens] | gi\|40039998 |

Peptide Information

| | Obsrv. | | | Start | End | |
|---|---|---|---|---|---|---|
| Calc. Mass | Mass | ±da | ±ppm | Seq. | Seq. | Sequence |
| 816.4322 | 816.3807 | −.0515 | −63 | 62 | 68 | LREEGGR |
| 826.3512 | 826.3818 | .0306 | 37 | 74 | 80 | ARSFCGE |
| 836.3745 | 836.3588 | −.0157 | −19 | 54 | 61 | ESGTEASR |
| 1598.8875 | 1598.7869 | −.1006 | −63 | 2 | 16 | TTFVGLRPQPGGR |
| | | | | | | GR |

| | |
|---|---|
| unnamed protein product [Homo sapiens] | gi\|7019891 |

Peptide Information

| | | | | Start | End | |
|---|---|---|---|---|---|---|
| Calc. Mass | Obsrv. Mass | ±da | ±ppm | Seq. | Seq. | Sequence |
| 808.4233 | 808.3741 | −.0492 | −61 | 253 | 259 | VTKMDSK |
| 820.4159 | 820.37 | −.0459 | −56 | 362 | 368 | VERSDSK |
| 824.4182 | 824.4018 | −.0164 | −20 | 253 | 259 | VTKMDSK |
| 830.4366 | 830.4167 | −.0199 | −24 | 478 | 484 | VPSDKER |
| 833.4224 | 833.4044 | −.018 | −22 | 413 | 420 | AEAGSRSR |
| 845.4264 | 845.4222 | −.0042 | −5 | 83 | 89 | LADHFSR |
| 848.4512 | 848.419 | −.0322 | −38 | 398 | 405 | FGLGTPEK |
| 848.4512 | 848.419 | −.0322 | −38 | 398 | 405 | FGLGTPEK |
| 858.454 | 858.3828 | −.0712 | −83 | 369 | 376 | SSPQAGRR |
| 858.454 | 858.3828 | −.0712 | −83 | 369 | 376 | SSPQAGRR |
| 1141.5848 | 1141.5339 | −.0509 | −45 | 20 | 29 | QDLIHASTEK |
| 1598.7917 | 1598.7869 | −.0048 | −3 | 377 | 391 | YGVQVMGSGLLAE |
| | | | | | | MK |
| 1914.9478 | 1914.8485 | −.0993 | −52 | 299 | 315 | SERPPTILMTEEPS |
| | | | | | | SPK |
| 1914.9478 | 1914.8485 | −.0993 | −52 | 299 | 315 | SERPPTILMTEEPS |
| | | | | | | SPK |

| | |
|---|---|
| Chain B, Crystal Structure Of The Complex Of | gi\|122921331 |
| Human Mitogen Activated Protein | |
| Kinase Kinase 5 Phox | |
| Domain (Map2k5-Phox) With | |
| Human Mitogen Activated | |
| Protein Kinase Kinase | |
| Kinase 3 (Map3k3b-Phox) | |

Peptide Information

| | Obsrv. | | | Start | End | |
|---|---|---|---|---|---|---|
| Calc. Mass | Mass | ±da | ±ppm | Seq. | Seq. | Sequence |
| 804.3417 | 804.3667 | .025 | 31 | 1 | 7 | SMGHSNR |
| 815.4621 | 815.4049 | −.0572 | −70 | 69 | 75 | AIDILDR |
| 845.4839 | 845.4222 | −.0617 | −73 | 8 | 14 | QSDVRIK |
| 847.3792 | 847.4076 | .0284 | 34 | 62 | 68 | NQDDL |
| | | | | | | DK |
| | 847.4076 | | | | | |
| 919.4156 | 919.4315 | .0159 | 17 | 32 | 38 | YEDVE |
| | | | | | | HK |

| | |
|---|---|
| 2-aminoethanethiol dioxygenase | gi\|62177110 |
| [Homo sapiens] | |

Protein Group

| | |
|---|---|
| 2-aminoethanethiol (cysteamine) | gi\|62739235 |
| dioxygenase [Homo sapiens] | |
| RecName: Full = 2-aminoethanethiol | gi\|88984104 |
| dioxygenase; | |
| AltName: Full = Cysteamine dioxygenase | |
| chromosome 10 open reading frame 22 | gi\|123237966 |
| [Homo sapiens] | |
| chromosome 10 open reading frame 22, isoform CRA_a | gi\|119574620 |
| [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 807.4723 | 807.4016 | −.0707 | −88 | 123 | 129 | VLYGTVR |
| 976.4555 | 976.5515 | .096 | 98 | 23 | 33 | GSGGGRGASDR |
| 998.5265 | 998.4788 | −.0477 | −48 | 63 | 71 | AEDLNIAPR |
| 998.5265 | 998.4788 | −.0477 | −48 | 63 | 71 | AEDLNIAPR |
| 1235.6677 | 1235.6796 | .0119 | 10 | 13 | 22 | IARQACLTFR |
| 1366.6646 | 1366.6088 | −.0558 | −41 | 16 | 28 | QACLTFRGSGGGR |
| 1914.9314 | 1914.8485 | −.0829 | −43 | 105 | 122 | SGTSIPLHDHPGMH |
| | | | | | | GMLK |
| 1914.9314 | 1914.8485 | −.0829 | −43 | 105 | 122 | SGTSIPLHDHPGMH |
| | | | | | | GMLK |

| | |
|---|---|
| Rap guanine nucleotide exchange factor (GEF) 1 | gi\|57208996 |
| [Homo sapiens] | |

Protein Group

| | |
|---|---|
| Rap guanine nucleotide exchange factor (GEF) 1 | gi\|55958113 |
| [Homo sapiens] | |

Peptide Information

| Calc. Mass | Obsrv. Mass | ±da | ±ppm | Start Seq. | End Seq. | Sequence |
|---|---|---|---|---|---|---|
| 815.4846 | 815.4049 | −.0797 | −98 | 2 | 9 | SGGLGLRR |
| 856.5039 | 856.5166 | .0127 | 15 | 38 | 44 | FHSPKIK |
| 907.4302 | 907.4031 | −.0271 | −30 | 9 | 16 | RSPEMSGK |
| 1037.5085 | 1037.5863 | .0778 | 75 | 25 | 33 | SHLSSFTMK |
| 1211.6783 | 1211.7719 | .0936 | 77 | 78 | 87 | EVVSALRYFK |
| | 1211.7719 | | | | | |
| 1211.6783 | 1211.7719 | .0936 | 77 | 78 | 87 | EVVSALRYFK |
| 1380.6101 | 1380.6191 | .009 | 7 | 147 | 158 | WSDQVMLEGVNS |
| 1540.7499 | 1540.6804 | −.0695 | −45 | 25 | 37 | SHLSSFTMKLMDK |

| | |
|---|---|
| vinculin isoform meta-VCL [Homo sapiens] | gi\|7669550 |

Protein Group

| | |
|---|---|
| RecName: Full = Vinculin; AltName: | gi\|21903479 |
| Full = Metavinculin | |
| vinculin, isoform CRA_b [Homo sapiens] | gi\|119574934 |
| vinculin, isoform CRA_b [Homo sapiens] | gi\|119574933 |

Peptide Information

| | | | | Start | End | |
|---|---|---|---|---|---|---|
| Calc. Mass | Obsrv. Mass | ±da | ±ppm | Seq. | Seq. | Sequence |
| 802.4417 | 802.3699 | −.0718 | −89 | 647 | 655 | AAAVGTANK |
| 803.4443 | 803.3769 | −.0674 | −84 | 1014 | 1020 | ALIQCAK |
| 804.4461 | 804.3667 | −.0794 | −99 | 269 | 276 | ALASIDSK |
| 807.3777 | 807.4016 | .0239 | 30 | 1039 | 1044 | QCTDKR |
| 815.4733 | 815.4049 | −.0684 | −84 | 410 | 417 | GALAEARK |
| 817.4236 | 817.3787 | −.0449 | −55 | 163 | 170 | NLGPGMTK |
| 818.4254 | 818.3765 | −.0489 | −60 | 1108 | 1115 | EAEAASIK |
| 820.4047 | 820.37 | −.0347 | −42 | 785 | 792 | AASDELSK |
| 824.4625 | 824.4018 | −.0607 | −74 | 489 | 496 | AAVHLEGK |
| 833.4185 | 833.4044 | −.0141 | −17 | 309 | 316 | VGELCAGK |
| 850.4087 | 850.3828 | −.0259 | −30 | 262 | 268 | DTEAMKR |
| 866.4036 | 866.402 | −.0016 | −2 | 262 | 268 | DTEAMKR |
| 976.5244 | 976.5515 | .0271 | 28 | 319 | 326 | REILGTCK |
| 1037.599 | 1037.5863 | −.0127 | −12 | 211 | 219 | IFVTTKNSK |
| 1229.7174 | 1229.837 | .1196 | 97 | 200 | 210 | ELLPVLISAMK |
| 1229.7174 | 1229.837 | .1196 | 97 | 200 | 210 | ELLPVLISAMK |
| 1235.6487 | 1235.6796 | .0309 | 25 | 189 | 199 | VMLVNSMNTVK |
| 1642.015 | 1641.8651 | −.1499 | −91 | 1056 | 1070 | IPTISTQLKILSTVK |

| | |
|---|---|
| BRCA1 [Homo sapiens] | gi\|237930356 |

Peptide Information

| Calc. | | | | Start | End | |
|---|---|---|---|---|---|---|
| Mass | Obsrv. Mass | ±da | ±ppm | Seq. | Seq. | Sequence |
| 800.3978 | 800.361 | −.0368 | −46 | 19 | 24 | WVVSYF |
| 812.4301 | 812.3808 | −.0493 | −61 | 11 | 18 | YFLGAGGK |
| 910.4087 | 910.4322 | .0235 | 26 | 1 | 7 | AEFVCER |

| | |
|---|---|
| myeloid/lymphoid or mixed-lineage | gi\|56202443 |
| leukemia (trithorax homolog, | |
| Drosophila | |
| sapiens] | |

Protein Group

| | |
|---|---|
| myeloid/lymphoid or mixed-lineage leukemia (trithorax | gi\|57209650 |
| homolog, Drosophila sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 818.3751 | 818.3765 | .0014 | 2 | 1 | 7 | GGRDEER |
| 820.3431 | 820.37 | .0269 | 33 | 237 | 244 | SSDGRPDS |
| 830.4003 | 830.4167 | .0164 | 20 | 138 | 145 | AQSNGPEK |
| 837.4036 | 837.434 | .0304 | 36 | 76 | 81 | FRPDMR |
| 847.3985 | 847.4076 | .0091 | 11 | 42 | 47 | FYFQDK |
| 848.4988 | 848.419 | −.0798 | −94 | 121 | 127 | EGRFVLK |
| 848.4988 | 848.419 | −.0798 | −94 | 121 | 127 | EGRFVLK |
| 866.4301 | 866.402 | −.0281 | −32 | 231 | 236 | RMQEFR |
| 1017.5323 | 1017.5444 | .0121 | 12 | 173 | 181 | EALRQAS |
| | | | | | | DK |

| | |
|---|---|
| unnamed protein product | gi\|164691141 |
| [Homo sapiens] | |

Peptide Information

| | Calc. | Obsrv. | | Start | End | |
|---|---|---|---|---|---|---|
| Mass | Mass | ±da | ±ppm | Seq. | Seq. | Sequence |
| 804.4461 | 804.3667 | −.0794 | −99 | 138 | 144 | SKEIEAK |
| 825.3988 | 825.3849 | −.0139 | −17 | 5 | 11 | LSGEYEK |
| 830.4981 | 830.4167 | −.0814 | −98 | 140 | 146 | EIEAKIK |
| 835.456 | 835.3892 | −.0668 | −80 | 71 | 77 | LFEELGK |
| 848.3706 | 848.419 | .0484 | 57 | 207 | 213 | LPEMDID |
| 848.3706 | 848.419 | .0484 | 57 | 207 | 213 | LPEMDID |
| 867.3843 | 867.3481 | −.0362 | −42 | 12 | 18 | FVSEDDR |
| 976.4655 | 976.5515 | .086 | 88 | 206 | 213 | KLPEMDID |

| | |
|---|---|
| biphenyl hydrolase-like (serine | gi\|119575525 |
| hydrolase; breast epithelial | |
| mucin-associated antigen), | |
| isoform CRA_c | |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 803.3795 | 803.3769 | −.0026 | −3 | 15 | 22 | AEGGAGWR |
| 807.3917 | 807.4016 | .0099 | 12 | 177 | 183 | DAVDLMK |
| 835.4196 | 835.3892 | −.0304 | −36 | 342 | 348 | LAEDFLQ |
| 853.4461 | 853.3766 | −.0695 | −81 | 1 | 8 | MGKHPAGR |
| 907.4672 | 907.4031 | −.0641 | −71 | 209 | 215 | YPSYIHK |
| 940.4921 | 940.4529 | −.0392 | −42 | 321 | 328 | LHLMPEGK |
| 1141.5498 | 1141.5339 | −.0159 | −14 | 155 | 164 | GYGHSRPP |
| | | | | | | DR |

| | |
|---|---|
| 58/C7 | Instr./Gel Origin |
| [1] Sample Project | |
| 20110503 | |
| | Instrument Sample |

| | | |
|---|---|---|
| Accession No. | Protein Name | Name |

| | |
|---|---|
| | KH5 (SEQ ID NO. KH5) |
| gi\|119626258 | PR domain containing 8, |
| | isoform CRA_b [Homo sapiens] |
| | Peptide Information |

| Calc. | Obsrv. | | | Start | End | |
|---|---|---|---|---|---|---|
| Mass | Mass | ±da | ±ppm | Seq. | Seq. | Sequence |
| 817.446 | 817.423 | −.023 | −28 | 149 | 155 | MNGRLAR |
| 818.4003 | 818.369 | −.0313 | −38 | 2 | 8 | EDTGIQR |
| 916.521 | 916.5026 | −.0184 | −20 | 91 | 98 | LVQSARDK |
| 959.4475 | 959.4648 | .0173 | 18 | 145 | 152 | SHNKMNGR |
| 965.4357 | 965.4838 | .0481 | 50 | 1 | 8 | MEDTGIQR |
| 1051.6047 | 1051.6862 | .0815 | 78 | 66 | 73 | RTVPYIFR |

| | |
|---|---|
| rhomboid 5 homolog 2 (Drosophila), | gi\|119609809 |
| isoform CRA_c [Homo sapiens] | |

Peptide Information

| Calc. Mass | Obsrv. Mass | ±da | ±ppm | Start Seq. | End Seq. | Sequence |
|---|---|---|---|---|---|---|
| 803.437 | 803.3735 | −.0635 | −79 | 198 | 204 | SDLGQKR |
| 817.4162 | 817.423 | .0068 | 8 | 151 | 156 | ERDLER |
| 870.4866 | 870.5401 | .0535 | 61 | 2 | 9 | MSLIPGPR |
| 920.4737 | 920.4175 | −.0562 | −61 | 20 | 26 | HFAFDRK |
| 1023.5469 | 1023.4952 | −.0517 | −51 | 103 | 111 | NKGVYESVK |
| 1026.5878 | 1026.4934 | −.0944 | −92 | 2 | 10 | MSLIPGPRR |
| 1156.6514 | 1156.6008 | −.0506 | −44 | 318 | 327 | LWLSLFLHAG |
| 1156.6514 | 1156.6008 | −.0506 | −44 | 318 | 327 | LWLSLFLHAG |
| 1933.8597 | 1933.9708 | .1111 | 57 | 187 | 203 | WQDDTGPPMD |
| | | | | | | KSDLGQK |
| 1933.8597 | 1933.9708 | .1111 | 57 | 187 | 203 | WQDDTGPPMD |
| | | | | | | KSDLGQK |

| | |
|---|---|
| centaurin, delta 2, isoform CRA_a | gi\|119595269 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 800.4737 | 800.4516 | −.0221 | −28 | 759 | 764 | LLQDRR |
| 807.4723 | 807.3954 | −.0769 | −95 | 1313 | 1318 | LYKEVR |
| 808.3981 | 808.4063 | .0082 | 10 | 994 | 1000 | KCGQTSK |
| 811.3944 | 811.4203 | .0259 | 32 | 250 | 256 | EEPPPSR |
| 815.4733 | 815.4612 | −.0121 | −15 | 1211 | 1217 | NVGIREK |
| 846.5196 | 846.4929 | −.0267 | −32 | 865 | 871 | LGRLPYK |
| 869.4475 | 869.3638 | −.0837 | −96 | 352 | 358 | LDTDHLR |
| 869.4475 | 869.3638 | −.0837 | −96 | 352 | 358 | LDTDHLR |
| 870.5659 | 870.5401 | −.0258 | −30 | 167 | 174 | LLVSLPTK |
| 885.4108 | 885.4137 | .0029 | 3 | 572 | 578 | RCAGEHR |
| 894.4427 | 894.3779 | −.0648 | −72 | 765 | 771 | AREEFSR |
| 913.4672 | 913.4619 | −.0053 | −6 | 637 | 643 | RCHLEAK |
| 933.4636 | 933.41 | −.0536 | −57 | 401 | 408 | AESDVERK |
| 1933.916 | 1933.9708 | .0548 | 28 | 1369 | 1383 | HEKQQCQA |
| | | | | | | FGIMWVR |
| 1934.0171 | 1933.9708 | −.0463 | −24 | 736 | 752 | HYSVVLPT |
| | | | | | | VSHSGFL |
| | | | | | | YK |

| | |
|---|---|
| unnamed protein product | gi\|193783575 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 826.4457 | 826.3953 | −.0504 | −61 | 7 | 13 | LPPGWEK |
| 856.4709 | 856.5273 | .0564 | 66 | 57 | 63 | CSHLLVK |
| 856.4709 | 856.5273 | .0564 | 66 | 57 | 63 | CSHLLVK |
| 890.4512 | 890.3947 | −.0565 | −63 | 128 | 136 | GAQGMGL |
| | | | | | | TR |
| 982.5468 | 982.4728 | −.074 | −75 | 7 | 14 | LPPGWEKR |
| 984.4421 | 984.4333 | −.0088 | −9 | 137 | 145 | LGDPYHP |
| | | | | | | EG |
| 1693.8439 | 1693.8403 | −.0036 | −2 | 120 | 136 | GDLGAFSR |
| | | | | | | GAGQGMG |
| | | | | | | LTR |

| | |
|---|---|
| immunoglobulin light chain | gi\|194173413 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 869.357 | 869.3638 | .0068 | 8 | 214 | 220 | SFNRGEC |
| 869.357 | 869.3638 | .0068 | 8 | 214 | 220 | SFNRGEC |
| 890.4366 | 890.3947 | −.0419 | −47 | 190 | 196 | ADYEKHK |
| 1156.582 | 1156.6008 | .0188 | 16 | 52 | 60 | LLMYWAS |
| | | | | | | TR |
| 1156.582 | 1156.6008 | .0188 | 16 | 52 | 60 | LLMYWAS |
| | | | | | | TR |
| 1933.9536 | 1933.9708 | .0172 | 9 | 1 | 18 | DIVMTQSP |
| | | | | | | DSLAVSLG |
| | | | | | | ER |
| 1933.9536 | 1933.9708 | .0172 | 9 | 1 | 18 | DIVMTQSP |
| | | | | | | DSLAVSLG |
| | | | | | | ER |
| 1946.027 | 1946.0345 | .0075 | 4 | 115 | 132 | TVAAPSVF |
| | | | | | | IFPPSDEQ |
| | | | | | | LK |

| | |
|---|---|
| hCG2041853 | gi\|119606879 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 848.4512 | 848.4214 | −.0298 | −35 | 29 | 35 | DFQPITK |
| 848.4512 | 848.4214 | −.0298 | −35 | 29 | 35 | DFQPITK |
| 863.4734 | 863.439 | −.0344 | −40 | 90 | 96 | TFKPTNR |
| 894.3951 | 894.3779 | −.0172 | −19 | 17 | 23 | EDFQEAR |
| 1003.5054 | 1003.4797 | −.0257 | −26 | 82 | 89 | EAEEREIK |
| 1022.4901 | 1022.4993 | .0092 | 9 | 17 | 24 | EDFQEARK |

| | |
|---|---|
| unknown [Homo sapiens] | gi\|41474316 |

Peptide Information

| | Obsrv. | | | Start | End | |
|---|---|---|---|---|---|---|
| Calc. Mass | Mass | ±da | ±ppm | Seq. | Seq. | Sequence |
| 807.3917 | 807.3954 | .0037 | 5 | 11 | 17 | MDTDVVK |
| 815.437 | 815.4612 | .0242 | 30 | 2 | 9 | SSPSPGKR |
| 920.4757 | 920.4175 | −.0582 | −63 | 11 | 18 | MDTDVVKL |
| 963.4928 | 963.4443 | −.0485 | −50 | 10 | 17 | RMDTDVVK |

| | |
|---|---|
| NF-kappa-B essential modulator isoform | gi\|53792133 |
| a [Homo sapiens] | |

Protein Group

| | |
|---|---|
| IKBKG protein [Homo sapiens] | gi\|116283880 |
| IKBKG protein [Homo sapiens] | |
| IKBKG protein [Homo sapiens] | gi\|29792042 |
| IKBKG protein [Homo sapiens] | gi\|12653067 |
| IKBKG protein [Homo sapiens] | gi\|15082399 |
| IkB kinase gamma subunit [Homo sapiens] | gi\|3641280 |
| NEMO protein [Homo sapiens] | gi\|5031141 |
| NF-KB Essential Modulator [Homo sapiens] | gi\|8249458 |
| NF-kB essential modulator NEMO [Homo sapiens] | gi\|9802304 |
| NF-kappa-B essential modulator isoform a [Homo | gi\|4504631 |
| sapiens] | |
| NFkappaB essential modulator [Homo sapiens] | gi\|16596512 |
| RecName: Full = NF-kappa-B essential modulator; | gi\|6685695 |
| Short = NEMO; AltName: | |
| Full = FIP-3; AltName: | |
| Full = IkB kinase-associated | |
| protein 1; Short = IKKAP1; | |
| AltName: Full = Inhibitor of | |
| nuclear factor kappa-B | |
| kinase subunit gamma; | |
| Short = I-kappa-B kinase | |
| subunit gamma; Short = IKK- | |
| gamma; Short = IKKG; | |
| Short = IkB kinase subunit | |
| gamma; AltName: Full = NF- | |
| kappa-B essential modifier | |
| inhibitor of kappa light polypeptide gene enhancer in | gi\|54696110 |
| B-cells, kinase gamma [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 807.4029 | 807.3954 | −.0075 | −9 | 353 | 358 | IEDMRK |
| 808.4199 | 808.4063 | −.0136 | −17 | 303 | 309 | AQADIYK |
| 836.3897 | 836.3643 | −.0254 | −30 | 310 | 316 | ADFQAER |
| 872.4948 | 872.4182 | −.0766 | −88 | 117 | 123 | QKEQALR |
| 908.389 | 908.4195 | .0305 | 34 | 345 | 352 | ASCQESAR |
| 933.4709 | 933.41 | −.0609 | −65 | 257 | 264 | GMQLEDLK |
| 948.5109 | 948.464 | −.0469 | −49 | 247 | 255 | SSVVGSERK |
| 959.5016 | 959.4648 | −.0368 | −38 | 174 | 182 | ARAASEQAR |
| 984.5196 | 984.4333 | −.0863 | −88 | 1 | 7 | MNRHLWK |
| 1934.0668 | 1933.9708 | −.096 | −50 | 293 | 309 | IVMETVPVLKAQADI |
| | | | | | | YK |
| 1934.0668 | 1933.9708 | −.096 | −50 | 293 | 309 | IVMETVPVLKAQADI |
| | | | | | | YK |

| | |
|---|---|
| helix-loop-helix protein transcription | gi\|84445 |
| factor 4 [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 817.4414 | 817.423 | −.0184 | −23 | 259 | 265 | SEKPQTK |
| 832.4206 | 832.4689 | .0483 | 58 | 228 | 234 | RMANNAR |
| 836.4148 | 836.3643 | −.0505 | −60 | 241 | 247 | DINEAFK |
| 848.4155 | 848.4214 | .0059 | 7 | 228 | 234 | RMANNAR |
| 848.4155 | 848.4214 | .0059 | 7 | 228 | 234 | RMANNAR |
| 870.4791 | 870.5401 | .061 | 70 | 285 | 291 | ERNLNPK |
| 895.364 | 895.3758 | .0118 | 13 | 189 | 196 | SDDESSQK |
| 945.4571 | 945.4341 | −.023 | −24 | 111 | 119 | SASMVGT |
| | | | | | | HR |
| 1051.5895 | 1051.6862 | .0967 | 92 | 56 | 64 | LDDAIHV |
| | | | | | | LR |

Peptide Information

| | Obsrv. | | | Start | | |
|---|---|---|---|---|---|---|
| Calc. Mass | Mass | ±da | ±ppm | Seq. | End Seq. | Sequence |
| 832.5291 | 832.4689 | −.0602 | −72 | 27 | 33 | LSIPKFK |
| 959.523 | 959.4648 | −.0582 | −61 | 34 | 41 | VEKMIDPK |
| 1023.5945 | 1023.4952 | −.0993 | −97 | 68 | 77 | GVALSNVVHK |
| 1095.631 | 1095.5862 | −.0448 | −41 | 119 | 127 | TRNIIFFGK |
| 1933.8121 | 1933.9708 | .1587 | 82 | 51 | 67 | HIFSEDTSDFSGMS |
| | | | | | | ETK |
| 1933.8121 | 1933.9708 | .1587 | 82 | 51 | 67 | HIFSEDTSDFSGMS |
| | | | | | | ETK |

| | |
|---|---|
| hCG1788266, iso form CRA_a [Homo sapiens] | gi\|119575210 |

Peptide Information

| Calc. | Obsrv. | | | Start | End | |
|---|---|---|---|---|---|---|
| Mass | Mass | ±da | ±ppm | Seq. | Seq. | Sequence |
| 807.3851 | 807.3954 | .0103 | 13 | 348 | 353 | MVICER |
| 811.4713 | 811.4203 | −.051 | −63 | 38 | 43 | FIFEKK |
| 817.4778 | 817.423 | −.0548 | −67 | 356 | 362 | NKSVELK |
| 856.4675 | 856.5273 | .0598 | 70 | 175 | 181 | FKAFSTR |
| 856.4675 | 856.5273 | .0598 | 70 | 175 | 181 | FKAFSTR |
| 890.4764 | 890.3947 | −.0817 | −92 | 2 | 9 | TMDALLAR |
| 894.4349 | 894.3779 | −.057 | −64 | 524 | 531 | ATTSVEMR |
| 908.5101 | 908.4195 | −.0906 | −100 | 444 | 451 | AGFLHHVK |
| 965.4686 | 965.4838 | .0152 | 16 | 182 | 189 | EDAEKFAR |
| 1022.5121 | 1022.4993 | −.0128 | −13 | 348 | 355 | MVICERSK |
| 1037.5118 | 1037.6041 | .0923 | 89 | 1 | 9 | MTMDALL |
| | | | | | | AR |

| | |
|---|---|
| inhibitor of kappa light polypeptide | gi\|11959308 |
| B-cells, kinase gamma, isoform | |
| Protein Group | |
| inhibitor of kappa light polypeptide | gi\|11959308 |
| B-cells, kinase gamma, isoform | |

Peptide Information

| | Obsrv. | | | Start | End | |
|---|---|---|---|---|---|---|
| Calc. Mass | Mass | ±da | ±ppm | Seq. | Seq. | Sequence |
| 807.4029 | 807.3954 | −.0075 | −9 | 168 | 173 | IEDMRK |
| 808.4199 | 808.4063 | −.0136 | −17 | 118 | 124 | AQADIYK |
| 836.3897 | 836.3643 | −.0254 | −30 | 125 | 131 | ADFQAER |
| 908.389 | 908.4195 | .0305 | 34 | 160 | 167 | ASCQESAR |
| 933.4709 | 933.41 | −.0609 | −65 | 72 | 79 | GMQLEDLK |
| 948.5109 | 948.464 | −.0469 | −49 | 62 | 70 | SSVVGSERK |
| 1934.0668 | 1933.9708 | −.096 | −50 | 108 | 124 | IVMETVPVLKAQADI |
| | | | | | | YK |
| 1934.0668 | 1933.9708 | −.096 | −50 | 108 | 124 | IVMETVPVLKAQADI |
| | | | | | | YK |

| | |
|---|---|
| Chain A, Solution Structure Of The C- | gi\|159163551 |
| Terminal Gal-Bind | |
| Lectin Domain From Human Galectin-4 | |

Peptide Information

| | | | | Start | | |
|---|---|---|---|---|---|---|
| Calc. Mass | Obsrv. Mass | ±da | ±ppm | Seq. | End Seq. | Sequence |
| 815.4734 | 815.4612 | −.0122 | −15 | 33 | 40 | LQGGLTAR |
| 818.4407 | 818.369 | −.0717 | −88 | 47 | 54 | GYVPPTGK |
| 826.4457 | 826.3953 | −.0504 | −61 | 55 | 61 | SFAINFK |
| 895.4454 | 895.3758 | −.0696 | −78 | 115 | 121 | CGLDRFK |
| 1945.9919 | 1946.0345 | .0426 | 22 | 98 | 114 | ITHNPFGPGQFFD |
| | | | | | | SIR |

| | |
|---|---|
| leucine zipper protein Fip3p [Homo sapiens] | gi\|3511151 |

Peptide Information

| | | | | Start | End | |
|---|---|---|---|---|---|---|
| Calc. Mass | Obsrv. Mass | ±da | ±ppm | Seq. | Seq. | Sequence |
| 807.4029 | 807.3954 | −.0075 | −9 | 353 | 358 | IEDMRK |
| 808.4199 | 808.4063 | −.0136 | −17 | 303 | 309 | AQADIYK |
| 836.3897 | 836.3643 | −.0254 | −30 | 310 | 316 | ADFQAER |
| 872.4948 | 872.4182 | −.0766 | −88 | 117 | 123 | QKEQALR |
| 908.389 | 908.4195 | .0305 | 84 | 345 | 352 | ASCQESAR |
| 933.4709 | 933.41 | −.0609 | −65 | 257 | 264 | GMQLEDLK |
| 948.5109 | 948.464 | −.0469 | −49 | 247 | 255 | SSVVGSERK |
| 959.5016 | 959.4648 | −.0368 | −38 | 174 | 182 | ARAASEQAR |
| 984.5196 | 984.4333 | −.0863 | −88 | 1 | 7 | MNRHLWK |
| 1934.0668 | 1933.9708 | −.096 | −50 | 293 | 309 | IVMETVPVLKAQADI |
| | | | | | | YK |
| 1934.0668 | 1933.9708 | −.096 | −50 | 293 | 309 | IVMETVPVLKAQADI |
| | | | | | | YK |

| | |
|---|---|
| Chain A, Crystal Structure Of Human Nemo Cc2_lz | gi\|226438304 |
| Domain | |

Protein Group

| | |
|---|---|
| Chain B, Crystal Structure Of Human Nemo Cc2_Iz | gi\|226438305 |
| Domain | |

Peptide Information

| | | | | Start | End | |
|---|---|---|---|---|---|---|
| Calc. Mass | Obsrv. Mass | ±da | ±ppm | Seq. | Seq. | Sequence |
| 808.4199 | 808.4063 | −.0136 | −17 | 62 | 68 | AQADIYK |
| 836.3897 | 836.3643 | −.0254 | −30 | 69 | 75 | ADFQAER |
| 933.4709 | 933.41 | −.0609 | −65 | 16 | 23 | GMQLEDLK |
| 948.5109 | 948.464 | −.0469 | −49 | 6 | 14 | SSVVGSERK |
| 1934.0668 | 1933.9708 | −.096 | −50 | 52 | 68 | IVMETVPVLKAQA [symbol] |
| | | | | | | YK |
| 1934.0668 | 1933.9708 | −.096 | −50 | 52 | 68 | IVMETVPVLKAQA [symbol] |
| | | | | | | YK |

| | |
|---|---|
| unnamed protein product [Homo sapiens] | gi\|194381172 |

[symbol]

indicates data missing or illegible when filed

Peptide Information

| Calc. | Obsrv. | | | Start | End | |
|---|---|---|---|---|---|---|
| Mass | Mass | ±da | ±ppm | Seq. | Seq. | Sequence |
| 817.4414 | 817.423 | −.0184 | −23 | 244 | 250 | SEKPQTK |
| 832.4206 | 832.4689 | .0483 | 58 | 213 | 219 | RMANNAR |
| 836.4148 | 836.3643 | −.0505 | −60 | 226 | 232 | DINEAFK |
| 848.4155 | 848.4214 | .0059 | 7 | 213 | 219 | RMANNAR |
| 848.4155 | 848.4214 | .0059 | 7 | 213 | 219 | RMANNAR |
| 870.4791 | 870.5401 | .061 | 70 | 270 | 276 | ERNLNPK |
| 895.364 | 895.3758 | .0118 | 13 | 174 | 181 | SDDESSQK |
| 945.4571 | 945.4341 | −.023 | −24 | 96 | 104 | SASMVGT |
| | | | | | | HR |
| 1051.5895 | 1051.6862 | .0967 | 92 | 41 | 49 | LDDAIHV |
| | | | | | | LR |

| | |
|---|---|
| NADH dehydrogenase [ubiquinone] | gi\|297206774 |
| 1 alpha subcomplex sub [symbol] | |

[symbol]

indicates data missing or illegible when filed

Peptide Information

| Calc. | Obsrv. | | | Start | End | |
|---|---|---|---|---|---|---|
| Mass | Mass | ±da | ±ppm | Seq. | Seq. | Sequence |
| 807.4359 | 807.3954 | −.0405 | −50 | 35 | 40 | DFIEKR |
| 826.4352 | 826.3953 | −.0399 | −48 | 21 | 26 | IHLCQR |
| 856.5363 | 856.5273 | −.009 | −11 | 14 | 20 | LGLREIR |
| 856.5363 | 856.5273 | −.009 | −11 | 14 | 20 | LGLREIR |
| | 856.5273 | | | | | |
| 870.5519 | 870.5401 | −.0118 | −14 | 9 | 17 | GVGAKLG |
| | | | | | | LR |
| 1022.553 | 1022.4993 | −.0537 | −53 | 65 | 72 | LWARYASR |

| | |
|---|---|
| unnamed protein product [Homo sapiens] | gi\|40038502 |

Peptide Information

| | | | | Start | End | |
|---|---|---|---|---|---|---|
| Calc. Mass | Obsrv. Mass | ±da | ±ppm | Seq. | Seq. | Sequence |
| 825.4287 | 825.3579 | −.0708 | −86 | 1 | 6 | MRFEVK |
| 870.5308 | 870.5401 | .0093 | 11 | 87 | 93 | AFLHVKR |
| 908.4724 | 908.4195 | −.0529 | −58 | 22 | 28 | DLYLQEK |
| 963.3911 | 963.4443 | .0532 | 55 | 14 | 21 | TCSSMFSK |
| 1811.9513 | 1811.8953 | −.056 | −31 | 42 | 56 | IMYTVSFIIHIDGFR |

| | |
|---|---|
| metallophosphoesterase 1, isoform | gi\|119621966 |
| CRA_a [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 807.4359 | 807.3954 | −.0405 | −50 | 171 | 176 | VERFEK |
| 815.437 | 815.4612 | .0242 | 30 | 286 | 292 | EASQKPR |
| 856.4498 | 856.5273 | .0775 | 90 | 139 | 144 | FQKMFR |
| 856.4498 | 856.5273 | .0775 | 90 | 139 | 144 | FQKMFR |
| 872.4447 | 872.4182 | −.0265 | −30 | 139 | 144 | FQKMFR |
| 890.4438 | 890.3947 | −.0491 | −55 | 226 | 233 | EQARGSSR |
| 894.3774 | 894.3779 | .0005 | 1 | 95 | 100 | EWQMER |
| 945.5265 | 945.4341 | −.0924 | −98 | 145 | 152 | HPSHVQLK |
| 1156.549 | 1156.6008 | .0518 | 45 | 1 | 10 | MAMIELG |
| | | | | | | FGR |
| 1156.549 | 1156.6008 | .0518 | 45 | 1 | 10 | MAMIELG |
| | | | | | | FGR |

| | |
|---|---|
| unnamed protein product [Homo sapiens] | gi\|40042838 |

Peptide Information

| Calc. | Obsrv. | | | Start | End | |
|---|---|---|---|---|---|---|
| Mass | Mass | ±da | ±ppm | Seq. | Seq. | Sequence |
| 800.4447 | 800.4516 | .0069 | 9 | 56 | 62 | VGMQIPR |
| 803.4193 | 803.3735 | −.0458 | −57 | 16 | 22 | SPKGCVR |
| 815.5349 | 815.4612 | −.0737 | −90 | 8 | 15 | KALVGLSK |
| 817.4202 | 817.423 | .0028 | 3 | 99 | 105 | GWNILTN |
| 1037.5006 | 1037.6041 | .1035 | 100 | 77 | 84 | MCLVEIEK |

| | 39/B12 | | Instr./Gel Origin |
|---|---|---|---|
| | [1] Sample Project | | Instrument |
| | 20110503 | | Sample |
| | | | Name |
| Protein Name- | KH6 (SEQ ID NO. | Accession No. | |
| | KH6) | | |

unnamed protein product [Homo sapiens]

Peptide Information

| | Obsrv. | | | Start | | |
|---|---|---|---|---|---|---|
| Calc. Mass | Mass | ±da | ±ppm | Seq. | End Seq. | Sequence |
| 813.4399 | 813.4365 | −.0034 | −4 | 349 | 355 | LHIMAGR |
| 828.4111 | 828.4147 | .0036 | 4 | 55 | 60 | SHKWDR |
| 1070.5742 | 1070.5751 | .0009 | 1 | 191 | 199 | GEVPPRYPR |
| 1141.5889 | 1141.5903 | .0014 | 1 | 310 | 320 | GGYTLVSGYPK |
| 1142.6317 | 1142.6177 | −.014 | −12 | 80 | 89 | QGHNSVFLIK |
| 1158.4707 | 1158.4716 | .0009 | 1 | 203 | 211 | DYFMPCPGR |
| 1158.4707 | 1158.4716 | .0009 | 1 | 203 | 211 | DYFMPCPGR |
| 1220.606 | 1220.6044 | −.0016 | −1 | 69 | 79 | NFPSPVDAAFR |
| 1220.606 | 1220.6044 | −.0016 | −1 | 69 | 79 | NFPSPVDAAFR |
| 1268.6747 | 1268.6769 | .0022 | 2 | 186 | 196 | FDPVRGEVPPR |
| 1268.6747 | 1268.6769 | .0022 | 2 | 186 | 196 | FDPVRGEVPPR |
| 1495.6787 | 1495.6715 | −.0072 | −5 | 175 | 185 | YYCFQGNQFLR |
| 1495.6787 | 1495.6715 | −.0072 | −5 | 175 | 185 | YYCFQGNQFLR |
| 1528.6672 | 1528.6715 | .0043 | 3 | 200 | 211 | DVRDYFMPCPGR |
| 1528.6672 | 1528.6715 | .0043 | 3 | 200 | 211 | DVRDYFMPCPGR |
| 1534.7802 | 1534.7703 | −.0099 | −6 | 67 | 79 | WKNFPSPVDAAFR |
| 1712.7698 | 1712.7891 | .0193 | 11 | 129 | 143 | GECQAEGVLFFQ |
| | | | | | | GDR |
| 2364.1653 | 2364.1553 | −.01 | −4 | 108 | 128 | LLQDEFPGIPSP |
| | | | | | | LDAAVE CHR |
| 2498.2344 | 2498.2322 | −.0022 | −1 | 325 | 348 | EVGTPHGIILDSV |
| | | | | | | DAAFIC |
| | | | | | | PGSSR |
| 2498.2344 | 2498.2322 | −.0022 | −1 | 325 | 348 | EVGTPHGIILDSV |
| | | | | | | DAAFIC |
| | | | | | | PGSSR |

| | |
|---|---|
| hemopexin, isoform CRA_b | gi\|119589125 |
| [Homo sapiens] | |

Protein Group

| | |
|---|---|
| RecName: Full = Hemopexin; | gi\|1708182 |
| AltName: | |
| Full = Beta-1B-glycoprotein; Flags: | |
| Precursor | |
| hemopexin [Homo sapiens] | gi\|184497 |
| hemopexin precursor [Homo sapiens] | gi\|11321561 |
| hemopexin precursor [Homo sapiens] | gi\|386789 |
| unnamed protein product [Homo sapiens] | gi\|189053897 |
| unnamed protein product [Homo sapiens] | gi\|80747621 |

Peptide Information

| | Obsrv. | | | Start | | |
|---|---|---|---|---|---|---|
| Calc. Mass | Mass | ±da | ±ppm | Seq. | End Seq. | Sequence |
| 813.4399 | 813.4365 | −.0034 | −4 | 372 | 378 | LHIMAGR |
| 828.4111 | 828.4147 | .0036 | 4 | 78 | 83 | SHKWDR |
| 1070.5742 | 1070.5751 | .0009 | 1 | 214 | 222 | GEVPPRYPR |
| 1141.5889 | 1141.5903 | .0014 | 1 | 333 | 343 | GGYTLVSGYPK |
| 1142.6317 | 1142.6177 | −.014 | −12 | 103 | 112 | QGHNSVFLIK |
| 1158.4707 | 1158.4716 | .0009 | 1 | 226 | 234 | DYFMPCPGR |
| 1158.4707 | 1158.4716 | .0009 | 1 | 226 | 234 | DYFMPCPGR |
| 1220.606 | 1220.6044 | −.0016 | −1 | 92 | 102 | NFPSPVDAAFR |
| 1220.606 | 1220.6044 | −.0016 | −1 | 92 | 102 | NFPSPVDAAFR |
| 1268.6747 | 1268.6769 | .0022 | 2 | 209 | 219 | FDPVRGEVPPR |
| 1268.6747 | 1268.6769 | .0022 | 2 | 209 | 219 | FDPVRGEVPPR |
| 1495.6787 | 1495.6715 | −.0072 | −5 | 198 | 208 | YYCFQGNQFLR |
| 1495.6787 | 1495.6715 | −.0072 | −5 | 198 | 208 | YYCFQGNQFLR |
| 1528.6672 | 1528.6715 | .0043 | 3 | 223 | 234 | DVRDYFMPCPGR |
| 1528.6672 | 1528.6715 | .0043 | 3 | 223 | 234 | DVRDYFMPCPGR |
| 1534.7802 | 1534.7703 | −.0099 | −6 | 90 | 102 | WKNFPSPVDAA |
| | | | | | | FR |
| 1712.7698 | 1712.7891 | .0193 | 11 | 152 | 166 | GECQAEGVLFF |
| | | | | | | QGDR |
| 2364.1653 | 2364.1553 | −.01 | −4 | 131 | 151 | LLQDEFPGIPSP |
| | | | | | | LDAAVE |
| | | | | | | CHR |
| 2498.2344 | 2498.2322 | −.0022 | −1 | 348 | 371 | EVGTPHGIILDSV |
| | | | | | | DAAFIC |
| | | | | | | PGSSR |
| 2498.2344 | 2498.2322 | −.0022 | −1 | 348 | 371 | EVGTPHGIILDSV |
| | | | | | | DAAFIC |
| | | | | | | PGSSR |

| | |
|---|---|
| HPX protein [Homo sapiens] | gi\|13529281 |

Peptide Information

| | | | | Start | | |
|---|---|---|---|---|---|---|
| Calc. Mass | Obsrv. Mass | ±da | ±ppm | Seq. | End Seq. | Sequence |
| 828.4111 | 828.4147 | .0036 | 4 | 78 | 83 | SHKWDR |
| 1070.5742 | 1070.5751 | .0009 | 1 | 214 | 222 | GEVPPRYPR |
| 1142.6317 | 1142.6177 | −.014 | −12 | 103 | 112 | QGHNSVFLIK |
| 1158.4707 | 1158.4716 | .0009 | 1 | 226 | 234 | DYFMPCPGR |
| 1158.4707 | 1158.4716 | .0009 | 1 | 226 | 234 | DYFMPCPGR |
| 1220.606 | 1220.6044 | −.0016 | −1 | 92 | 102 | NFPSPVDAAFR |
| 1220.606 | 1220.6044 | −.0016 | −1 | 92 | 102 | NFPSPVDAAFR |
| 1268.6747 | 1268.6769 | .0022 | 2 | 209 | 219 | FDPVRGEVPPR |
| 1268.6747 | 1268.6769 | .0022 | 2 | 209 | 219 | FDPVRGEVPPR |
| 1495.6787 | 1495.6715 | −.0072 | −5 | 198 | 208 | YYCFQGNQFLR |
| 1495.6787 | 1495.6715 | −.0072 | −5 | 198 | 208 | YYCFQGNQFLR |
| 1528.6672 | 1528.6715 | .0043 | 3 | 223 | 234 | DVRDYFMPCPGR |
| 1528.6672 | 1528.6715 | .0043 | 3 | 223 | 234 | DVRDYFMPCPGR |
| 1534.7802 | 1534.7703 | −.0099 | −6 | 90 | 102 | WKNFPSPVDAA |
| | | | | | | FR |
| 1712.7698 | 1712.7891 | .0193 | 11 | 152 | 166 | GECQAEGVLFF |
| | | | | | | QGDR |
| 2364.1653 | 2364.1553 | −.01 | −4 | 131 | 151 | LLQDEFPGIPSP |
| | | | | | | LDAAVE |
| | | | | | | CHR |

| | |
|---|---|
| hemopexin, isoform CRA_d [Homo sapiens] | gi\|119589127 |

Peptide Information

| | | | | Start | | |
|---|---|---|---|---|---|---|
| Calc. Mass | Obsrv. Mass | ±da | ±ppm | Seq. | End Seq. | Sequence |
| 813.4399 | 813.4365 | −.0034 | −4 | 301 | 307 | LHIMAGR |
| 828.4111 | 828.4147 | .0036 | 4 | 78 | 83 | SHKWDR |
| 1141.5889 | 1141.5903 | .0014 | 1 | 262 | 272 | GGYTLVSGYPK |
| 1142.6317 | 1142.6177 | −.014 | −12 | 103 | 112 | QGHNSVFLIK |
| 1220.606 | 1220.6044 | −.0016 | −1 | 92 | 102 | NFPSPVDAAFR |
| 1220.606 | 1220.6044 | −.0016 | −1 | 92 | 102 | NFPSPVDAAFR |
| 1534.7802 | 1534.7703 | −.0099 | −6 | 90 | 102 | WKNFPSPVDAAFR |
| 2364.1653 | 2364.1553 | −.01 | −4 | 131 | 151 | LLQDEFPGIPSP |
| | | | | | | LDAAVE |
| | | | | | | CHR |
| 2498.2344 | 2498.2322 | −.0022 | −1 | 277 | 300 | EVGTPHGIILDSV |
| | | | | | | DAAFIC |
| | | | | | | PGSSR |
| 2498.2344 | 2498.2322 | −.0022 | −1 | 277 | 300 | EVGTPHGIILDSV |
| | | | | | | DAAFIC |
| | | | | | | PGSSR |

| | |
|---|---|
| hemopexin, isoform CRA_a [Homo sapiens] | gi\|119589124 |

Peptide Information

| | | | | Start | | |
|---|---|---|---|---|---|---|
| Calc. Mass | Obsrv. Mass | ±da | ±ppm | Seq. | End Seq. | Sequence |
| 813.4399 | 813.4365 | −.0034 | −4 | 118 | 124 | LHIMAGR |
| 1141.5889 | 1141.5903 | .0014 | 1 | 79 | 89 | GGYTLVSGYPK |
| 2498.2344 | 2498.2322 | −.0022 | −1 | 94 | 117 | EVGTPHGIILDSV |
| | | | | | | DAAFIC |
| | | | | | | PGSSR |
| 2498.2344 | 2498.2322 | −.0022 | −1 | 94 | 117 | EVGTPHGIILDSV |
| | | | | | | DAAFIC |
| | | | | | | PGSSR |

| | |
|---|---|
| hemopexin | gi\|226337 |

Peptide Information

| | Obsrv. | | | Start | | |
|---|---|---|---|---|---|---|
| Calc. Mass | Mass | ±da | ±ppm | Seq. | End Seq. | Sequence |
| 1141.5889 | 1141.5903 | .0014 | 1 | 78 | 88 | GGYTLVSGYPK |
| 2498.2344 | 2498.2322 | −.0022 | −1 | 93 | 116 | EVGTPHGIILDSV |
| | | | | | | DAAFIC |
| | | | | | | PGSSR |
| 2498.2344 | 2498.2322 | −.0022 | −1 | 93 | 116 | EVGTPHGIILDSV |
| | | | | | | DAAFIC |
| | | | | | | PGSSR |

| | |
|---|---|
| hemopexin, isoform CRA_c [Homo sapiens] | gi\|119589126 |

Peptide Information

| | | | | Start | | |
|---|---|---|---|---|---|---|
| Calc. Mass | Obsrv. Mass | ±da | ±ppm | Seq. | End Seq. | Sequence |
| 813.4399 | 813.4365 | −.0034 | −4 | 170 | 176 | LHIMAGR |
| 1141.5889 | 1141.5903 | .0014 | 1 | 131 | 141 | GGYTLVSGYPK |
| 2498.2344 | 2498.2322 | −.0022 | −1 | 146 | 169 | EVGTPHGIILDSV |
| | | | | | | DAAFIC |
| | | | | | | PGSSR |
| 2498.2344 | 2498.2322 | −.0022 | −1 | 146 | 169 | EVGTPHGIILDSV |
| | | | | | | DAAFIC |
| | | | | | | PGSSR |

| | |
|---|---|
| unnamed protein product [Homo sapiens] | gi\|221044726 |

Peptide Information

| | Obsrv. | | | | | |
|---|---|---|---|---|---|---|
| Calc. Mass | Mass | ±da | ±ppm | Start Seq. | End Seq. | Sequence |
| 828.4111 | 828.4147 | .0036 | 4 | 78 | 83 | SHKWDR |
| 1142.6317 | 1142.6177 | −.014 | −12 | 103 | 112 | QGHNSVFLIK |
| 1220.606 | 1220.6044 | −.0016 | −1 | 92 | 102 | NFPSPVDAAFR |
| 1220.606 | 1220.6044 | −.0016 | −1 | 92 | 102 | NFPSPVDAAFR |

| | |
|---|---|
| albumin, | gi\|119626074 |
| isoform CRA_k [Homo | |
| sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 875.5098 | 875.5068 | −.003 | −3 | 51 | 57 | LSQRFPK |
| 960.5625 | 960.5635 | .001 | 1 | 235 | 242 | FQNALLVR |
| 1084.6586 | 1084.5902 | −.0684 | −63 | 45 | 54 | ALAVARLSQR |
| 1084.6586 | 1084.5902 | −.0684 | −63 | 45 | 54 | ALAVARLSQR |
| 1141.694 | 1141.5903 | −.1037 | −91 | 406 | 417 | KLVAASQAALGL |
| 1149.5759 | 1149.6154 | .0395 | 34 | 25 | 34 | DAHKSEVAHR |
| 1311.7419 | 1311.6891 | −.0528 | −40 | 170 | 180 | HPDYSVVLLLR |
| 1358.6298 | 1358.6514 | .0216 | 16 | 378 | 389 | AVMDDFAAFVEK |
| 1467.8431 | 1467.8401 | −.003 | −2 | 169 | 180 | RHPDYSVVLLLR |
| 1511.8429 | 1511.7273 | −.1156 | −76 | 247 | 260 | VPQVSTPTLVE |
| | | | | | | VSR |
| 1546.7968 | 1546.7734 | −.0234 | −15 | 107 | 118 | LKECCEKPLLEK |
| 1639.9379 | 1639.8716 | −.0663 | −40 | 246 | 260 | KVPQVSTPTLV |
| | | | | | | EVSR |
| 1684.821 | 1684.8859 | .0649 | 39 | 95 | 108 | YICENQDSISS |
| | | | | | | KLK |
| 1684.821 | 1684.8859 | .0649 | 39 | 95 | 108 | YICENQDSISS |
| | | | | | | KLK |
| 1910.9318 | 1910.9291 | −.0027 | −1 | 317 | 332 | RPCFSALEVDE |
| | | | | | | TYVPK |

| | |
|---|---|
| unnamed protein product [Homo sapiens] | gi\|194391112 |

Peptide Information

Protein Group

| | | | | Start | End | |
|---|---|---|---|---|---|---|
| Calc. Mass | Obsrv. Mass | ±da | ±ppm | Seq. | Seq. | Sequence |
| 875.5098 | 875.5068 | −.003 | −3 | 157 | 163 | LSQRFPK |
| 927.4934 | 927.4922 | −.0012 | −1 | 76 | 82 | YLYEIAR |
| 960.5625 | 960.5635 | .001 | 1 | 341 | 348 | FQNALLVR |
| 1141.694 | 1141.5903 | −.1037 | −91 | 512 | 523 | KLVAASQAALGL |
| 1149.5759 | 1149.6154 | .0395 | 34 | 25 | 34 | DAHKSEVAHR |
| 1220.6885 | 1220.6044 | −.0841 | −69 | 66 | 75 | LVNEETFLKK |
| 1220.6885 | 1220.6044 | −.0841 | −69 | 66 | 75 | LVNEETFLKK |
| 1311.7419 | 1311.6891 | −.0528 | −40 | 276 | 286 | HPDYSVVLLLR |
| 1358.6298 | 1358.6514 | .0216 | 16 | 484 | 495 | AVMDDFAAFVEK |
| 1467.8431 | 1467.8401 | −.003 | −2 | 275 | 286 | RHPDYSVVLLLR |
| 1511.8429 | 1511.7273 | −.1156 | −76 | 353 | 366 | VPQVSTPTLVEVSR |
| 1546.7968 | 1546.7734 | −.0234 | −15 | 213 | 224 | LKECCEKPLLEK |
| 1639.9379 | 1639.8716 | −.0663 | −40 | 352 | 366 | KVPQVSTPTLVEV |
| | | | | | | SR |
| 1684.821 | 1684.8859 | .0649 | 39 | 201 | 214 | YICENQDSISSKLK |
| 1684.821 | 1684.8859 | .0649 | 39 | 201 | 214 | YICENQDSISSKLK |
| 1910.9318 | 1910.9291 | −.0027 | −1 | 423 | 438 | RPCFSALEVDETY |
| | | | | | | VPK |

| | |
|---|---|
| albumin, isoform CRA_m [Homo | gi\|119626076 |
| sapiens] | |
| PRO2619 [Homo sapiens] | gi\|11493459 |

Peptide Information

| | Obsrv. | | | Start | | |
|---|---|---|---|---|---|---|
| Calc. Mass | Mass | ±da | ±ppm | Seq. | End Seq. | Sequence |
| 875.5098 | 875.5068 | −.003 | −3 | 133 | 139 | LSQRFPK |
| 927.4934 | 927.4922 | −.0012 | −1 | 52 | 58 | YLYEIAR |
| 960.5625 | 960.5635 | .001 | 1 | 317 | 324 | FQNALLVR |
| 1141.694 | 1141.5903 | −.1037 | −91 | 488 | 499 | KLVAASQAALGL |
| 1311.7419 | 1311.6891 | −.0528 | −40 | 252 | 262 | HPDYSVVLLLR |
| 1358.6298 | 1358.6514 | .0216 | 16 | 460 | 471 | AVMDDFAAFV |
| | | | | | | EK |
| 1467.8431 | 1467.8401 | −.003 | −2 | 251 | 262 | RHPDYSVVLLLR |
| 1510.6083 | 1510.6826 | .0743 | 49 | 1 | 12 | MADCCAKQEP |
| | | | | | | ER |
| 1511.8429 | 1511.7273 | −.1156 | −76 | 329 | 342 | VPQVSTPTLVE |
| | | | | | | VSR |
| 1546.7968 | 1546.7734 | −.0234 | −15 | 189 | 200 | LKECCEKPLLEK |
| 1639.9379 | 1639.8716 | −.0663 | −40 | 328 | 342 | KVPQVSTPTLV |
| | | | | | | EVSR |
| 1684.821 | 1684.8859 | .0649 | 39 | 177 | 190 | YICENQDSISS |
| | | | | | | KLK |
| 1684.821 | 1684.8859 | .0649 | 39 | 177 | 190 | YICENQDSISS |
| | | | | | | KLK |
| 1910.9318 | 1910.9291 | −.0027 | −1 | 399 | 414 | RPCFSALEVDE |
| | | | | | | TYVPK |

ALB protein [Homo sapiens]

Protein Group

| | |
|---|---|
| growth-inhibiting protein 20 [Homo sapiens] | gi\|27692693 |
| | gi\|47118015 |

Peptide Information

| | | | | Start | End | |
|---|---|---|---|---|---|---|
| Calc. Mass | Obsrv. Mass | ±da | ±ppm | Seq. | Seq. | Sequence |
| 875.5098 | 875.5068 | −.003 | −3 | 51 | 57 | LSQRFPK |
| 960.5625 | 960.5635 | .001 | 1 | 235 | 242 | FQNALLVR |
| 1141.694 | 1141.5903 | −.1037 | −91 | 406 | 417 | KLVAASQAALGL |
| 1149.5759 | 1149.6154 | .0395 | 34 | 25 | 34 | DAHKSEVAHR |
| 1311.7419 | 1311.6891 | −.0528 | −40 | 170 | 180 | HPDYSVVLLLR |
| 1358.6298 | 1358.6514 | .0216 | 16 | 378 | 389 | AVMDDFAAFVEK |
| 1467.8431 | 1467.8401 | −.003 | −2 | 169 | 180 | RHPDYSVVLLLR |
| 1511.8429 | 1511.7273 | −.1156 | −76 | 247 | 260 | VPQVSTPTLVEVSR |
| 1546.7968 | 1546.7734 | −.0234 | −15 | 107 | 118 | LKECCEKPLLEK |
| 1639.9379 | 1639.8716 | −.0663 | −40 | 246 | 260 | KVPQVSTPTLVEV |
| | | | | | | SR |
| 1684.821 | 1684.8859 | .0649 | 39 | 95 | 108 | YICENQDSISSKLK |
| 1684.821 | 1684.8859 | .0649 | 39 | 95 | 108 | YICENQDSISSKLK |
| 1910.9318 | 1910.9291 | −.0027 | −1 | 317 | 332 | RPCFSALEVDETY |
| | | | | | | VPK |

| | |
|---|---|
| Chain A, Human Serum Albumin Mutant R218h | gi\|31615330 |
| Complexed With | |
| Thyroxine (3,3′,5,5′- | |
| Tetraiodo-L-Thyronine) | |

Protein Group

| | |
|---|---|
| Chain A, Human Serum Albumin Mutant R218h | gi\|31615333 |
| Complexed With Thyroxine | |
| (3,3′,5,5′-Tetraiodo-L-Thyronine) And Myristic Acid | |
| (Tetradecanoic Acid) | |

Peptide Information

| | Obsrv. | | | Start | | |
|---|---|---|---|---|---|---|
| Calc. Mass | Mass | ±da | ±ppm | Seq. | End Seq. | Sequence |
| 927.4934 | 927.4922 | −.0012 | −1 | 138 | 144 | YLYEIAR |
| 960.5625 | 960.5635 | .001 | 1 | 403 | 410 | FQNALLVR |
| 1141.694 | 1141.5903 | −.1037 | −91 | 574 | 585 | KLVAASQAALGL |
| 1149.615 | 1149.6154 | .0004 | 0 | 42 | 51 | LVNEVTEFAK |
| 1311.7419 | 1311.6891 | −.0528 | −40 | 338 | 348 | HPDYSVVLLLR |
| 1358.6298 | 1358.6514 | .0216 | 16 | 546 | 557 | AVMDDFAAFVEK |
| 1467.8431 | 1467.8401 | −.003 | −2 | 337 | 348 | RHPDYSVVLLLR |
| 1510.8278 | 1510.6826 | −.1452 | −96 | 213 | 225 | AWAVAHLSQRFPK |
| 1511.8429 | 1511.7273 | −.1156 | −76 | 415 | 428 | VPQVSTPTLVEVSR |
| 1546.7968 | 1546.7734 | −.0234 | −15 | 275 | 286 | LKECCEKPLLEK |
| 1639.9379 | 1639.8716 | −.0663 | −40 | 414 | 428 | KVPQVSTPTLVEV |
| | | | | | | SR |
| 1684.821 | 1684.8859 | .0649 | 39 | 263 | 276 | YICENQDSISSKLK |
| 1684.821 | 1684.8859 | .0649 | 39 | 263 | 276 | YICENQDSISSKLK |
| 1910.9318 | 1910.9291 | −.0027 | −1 | 485 | 500 | RPCFSALEVDETY |
| | | | | | | VPK |

| | |
|---|---|
| Chain A, Human Serum Albumin Complexed With | gi\|122920512 |
| Myristate And Aspirin | |

Peptide Information

| | | | | Start | End | |
|---|---|---|---|---|---|---|
| Calc. Mass | Obsrv. Mass | ±da | ±ppm | Seq. | Seq. | Sequence |
| 875.5098 | 875.5068 | −.003 | −3 | 219 | 225 | LSQRFPK |
| 927.4934 | 927.4922 | −.0012 | −1 | 138 | 144 | YLYEIAR |
| 960.5625 | 960.5635 | .001 | 1 | 403 | 410 | FQNALLVR |
| 1141.694 | 1141.5903 | −.1037 | −91 | 574 | 585 | KLVAASQAALGL |
| 1149.615 | 1149.6154 | .0004 | 0 | 42 | 51 | LVNEVTEFAK |
| 1311.7419 | 1311.6891 | −.0528 | −40 | 338 | 348 | HPDYSVVLLLR |
| 1358.6298 | 1358.6514 | .0216 | 16 | 546 | 557 | AVMDDFAAFVEK |
| 1467.8431 | 1467.8401 | −.003 | −2 | 337 | 348 | RHPDYSVVLLLR |
| 1511.8429 | 1511.7273 | −.1156 | −76 | 415 | 428 | VPQVSTPTLVEVSR |
| 1546.7968 | 1546.7734 | −.0234 | −15 | 275 | 286 | LKECCEKPLLEK |
| 1639.9379 | 1639.8716 | −.0663 | −40 | 414 | 428 | KVPQVSTPTLVEV |
| | | | | | | SR |
| 1684.821 | 1684.8859 | .0649 | 39 | 263 | 276 | YICENQDSISSKLK |
| 1684.821 | 1684.8859 | .0649 | 39 | 263 | 276 | YICENQDSISSKLK |
| 1910.9318 | 1910.9291 | −.0027 | −1 | 485 | 500 | RPCFSALEVDETY |
| | | | | | | VPK |

| | |
|---|---|
| Chain A, Human Serum Albumin | gi\|311771978 |
| Complexed With | |
| Dansyl-L-Asparagine And Myristic Acid | |

Protein Group

| | |
|---|---|
| Chain A, Human Serum Albumin | gi\|311771979 |
| Complexed With | |
| Dansyl-L-Arginine And Myristic Acid | |
| Chain A, Human Serum Albumin | gi\|311771969 |
| Complexed With | |
| Dansyl-L-Glutamate And Myristic Acid | |
| Chain A, Human Serum Albumin | gi\|78101694 |
| Complexed With | |
| Myristate And Azapropazone | |
| Chain A, Human Serum Albumin | gi\|78101695 |
| Complexed With | |
| Myristate, Azapropazone And | |
| Indomethacin | |
| Chain A, Human Serum Albumin | gi\|78101701 |
| Complexed With | |
| Myristate, Phenylbutazone And | |
| Indomethacin | |

Peptide Information

| | Obsrv. | | | Start | | |
|---|---|---|---|---|---|---|
| Calc. Mass | Mass | ±da | ±ppm | Seq. | End Seq. | Sequence |
| 875.5098 | 875.5068 | −.003 | −3 | 219 | 225 | LSQRFPK |
| 927.4934 | 927.4922 | −.0012 | −1 | 138 | 144 | YLYEIAR |
| 960.5625 | 960.5635 | .001 | 1 | 403 | 410 | FQNALLVR |
| 1141.694 | 1141.5903 | −.1037 | −91 | 574 | 585 | KLVAASQAALGL |
| 1149.615 | 1149.6154 | .0004 | 0 | 42 | 51 | LVNEVTEFAK |
| 1311.7419 | 1311.6891 | −.0528 | −40 | 338 | 348 | HPDYSVVLLLR |
| 1358.6298 | 1358.6514 | .0216 | 16 | 546 | 557 | AVMDDFAAFVEK |
| 1467.8431 | 1467.8401 | −.003 | −2 | 337 | 348 | RHPDYSVVLLLR |
| 1511.8429 | 1511.7273 | −.1156 | −76 | 415 | 428 | VPQVSTPTLVEVSR |
| 1546.7968 | 1546.7734 | −.0234 | −15 | 275 | 286 | LKECCEKPLLEK |
| 1639.9379 | 1639.8716 | −.0663 | −40 | 414 | 428 | KVPQVSTPTLVEV |
| | | | | | | SR |
| 1684.821 | 1684.8859 | .0649 | 39 | 263 | 276 | YICENQDSISSKLK |
| 1684.821 | 1684.8859 | .0649 | 39 | 263 | 276 | YICENQDSISSKLK |
| 1910.9318 | 1910.9291 | −.0027 | −1 | 485 | 500 | RPCFSALEVDETY |
| | | | | | | VPK |

| | |
|---|---|
| Chain A, Human Serum Albumin Complexed With | gi\|311771982 |
| Dansyl-L-Norvaline | |

Protein Group

| | |
|---|---|
| Chain A, Human Serum Albumin Complexed With | gi\|311771976 |
| Dansyl-L-Asparagine | |
| Chain A, Human Serum Albumin Complexed With | gi\|311771980 |
| Dansyl-L-Phenylalanine | |
| Chain A, Human Serum Albumin Complexed With | gi\|311771974 |
| Dansyl-L-Sarcosine | |
| Chain A, Human Serum Albumin Complexed With | gi\|330689346 |
| Iophenoxic Acid | |
| Chain A, Human Serum Albumin In Complex With | gi\|310689763 |
| Compound 1 | |
| Chain A, Human Serum Albumin In Complex With | gi\|310689765 |
| Compound 2 | |
| Chain A, Human Serum Albumin In Complex With | gi\|310689767 |
| Compound 3 | |
| Chain A, Human Serum Albumin With Bound Sulfate | gi\|294979706 |
| Chain B, Human Serum Albumin Complexed With | gi\|311771977 |
| Dansyl-L-Asparagine | |
| Chain B, Human Serum Albumin Complexed With | gi\|311771983 |
| Dansyl-L-Norvaline | |
| Chain B, Human Serum Albumin Complexed With | gi\|311771981 |
| Dansyl-L-Phenylalanine | |
| Chain B, Human Serum Albumin Complexed With | gi\|311771975 |
| Dansyl-L-Sarcosine | |
| Chain B, Human Serum Albumin Complexed With | gi\|330689347 |
| Iophenoxic Acid | |
| Chain B, Human Serum Albumin In Complex With | gi\|310689764 |
| Compound 1 | |
| Chain B, Human Serum Albumin In Complex With | gi\|310689766 |
| Compound 2 | |
| Chain B, Human Serum Albumin In Complex With | gi\|310689768 |
| Compound 3 | |
| Chain B, Human Serum Albumin With Bound Sulfate | gi\|294979707 |
| unnamed protein product [Homo sapiens] | gi\|317439073 |
| unnamed protein product [Homo sapiens] | gi\|311843689 |
| unnamed protein product [Homo sapiens] | gi\|311080790 |

Peptide Information

| | | | | Start | End | |
|---|---|---|---|---|---|---|
| Calc. Mass | Obsrv. Mass | ±da | ±ppm | Seq. | Seq. | Sequence |
| 875.5098 | 875.5068 | −.003 | −3 | 219 | 225 | LSQRFPK |
| 927.4934 | 927.4922 | −.0012 | −1 | 138 | 144 | YLYEIAR |
| 960.5625 | 960.5635 | .001 | 1 | 403 | 410 | FQNALLVR |
| 1141.694 | 1141.5903 | −.1037 | −91 | 574 | 585 | KLVAASQAALGL |
| 1149.615 | 1149.6154 | .0004 | 0 | 42 | 51 | LVNEVTEFAK |
| 1311.7419 | 1311.6891 | −.0528 | −40 | 338 | 348 | HPDYSVVLLLR |
| 1358.6298 | 1358.6514 | .0216 | 16 | 546 | 557 | AVMDDFAAFVEK |
| 1467.8431 | 1467.8401 | −.003 | −2 | 337 | 348 | RHPDYSVVLLLR |
| 1511.8429 | 1511.7273 | −.1156 | −76 | 415 | 428 | VPQVSTPTLVEVSR |
| 1546.7968 | 1546.7734 | −.0234 | −15 | 275 | 286 | LKECCEKPLLEK |
| 1639.9379 | 1639.8716 | −.0663 | −40 | 414 | 428 | KVPQVSTPTLVEV |
| | | | | | | SR |
| 1684.821 | 1684.8859 | .0649 | 39 | 263 | 276 | YICENQDSISSKLK |
| 1684.821 | 1684.8859 | .0649 | 39 | 263 | 276 | YICENQDSISSKLK |
| 1910.9318 | 1910.9291 | −.0027 | −1 | 485 | 500 | RPCFSALEVDETY |
| | | | | | | VPK |

| | |
|---|---|
| importin 4 variant [Homo sapiens] | gi\|62088532 |

Peptide Information

| Calc. | | | | | | |
|---|---|---|---|---|---|---|
| Mass | Obsrv. | ±da | ±ppm | Star Seq. | End Seq. | Sequence |
| 1220.5867 | 1220.6044 | .0177 | 15 | 97 | 108 | SSSSPTQLGNSR |

| | |
|---|---|
| NEDD4 family-interacting protein 2 isoform | gi\|2390467 |
| sapiens] | |

Protein Group

| | |
|---|---|
| KIAA1165 protein [Homo sapiens] | gi\|6330171 |
| NDFIP2 protein [Homo sapiens] | gi\|19913452 |
| NDFIP2 protein [Homo sapiens] | gi\|18314412 |
| NEDD4 family-interacting protein 2 isoform 2 [Homo | gi\|239046743 |
| sapiens] | |
| Nedd4 family interacting protein 2 [Homo sapiens] | gi\|119601005 |
| Nedd4 family interacting protein 2 [Homo sapiens] | gi\|55661722 |
| Nedd4 family interacting protein 2 [Homo sapiens] | gi\|55663185 |
| RecName: Full = NEDD4 family-interacting protein 2; | gi\|73921209 |
| AltName: Full = NEDD4 WW domain-binding protein | |
| 5A; | |
| AltName: Full = Putative MAPK-activating protein | |
| PM04/PM05/PM06/PM07; AltName: Full = Putative | |
| NF-kappa-B-activating protein 413 | |
| putative MAPK activating protein [Homo sapiens] | gi\|31455517 |
| putative MAPK activating protein [Homo sapiens] | gi\|31455511 |
| putative MAPK activating protein [Homo sapiens] | gi\|31455515 |
| putative MAPK activating protein [Homo sapiens] | gi\|31455513 |
| putative NFkB activating protein [Homo sapiens] | gi\|31455493 |
| unnamed protein product [Homo sapiens] | gi\|194386240 |
| unnamed protein product [Homo sapiens] | gi\|40032123 |
| unnamed protein product [Homo sapiens] | gi\|194378394 |
| unnamed protein product [Homo sapiens] | gi\|7023163 |

Peptide Information

| | Obsrv. | | | | | |
|---|---|---|---|---|---|---|
| Calc. Mass | Mass | ±da | ±ppm | Start Seq. | End Seq. | Sequence |
| 1220.5146 | 1220.6044 | .0898 | 74 | 319 | 329 | NMSESMAAAHR |

| | |
|---|---|
| Chain E, Crystal Structure Of Murine Thrombin | gi\|251837019 |
| Mutant W215aE217A IN Complex With The | |
| Extracellular Fragment Of Human Par1 | |

Protein Group

| | |
|---|---|
| Chain 2, Crystal Structure Of Human Alpha-Thrombin | gi\|4699691 |
| Inhibited With Sel2770. | |
| Chain 2, Human Alpha Thrombin Inhibited By Rppgf | gi\|28949036 |
| And Hirugen | |
| Chain 2, Selective Non Electrophilic Thrombin | gi\|10835495 |
| Inhibitors | |
| With Cyclohexyl Moieties. | |
| Chain 2, Selective Non Electrophilic Thrombin | gi\|10835492 |
| Inhibitors | |
| With Cyclohexyl Moieties. | |
| Chain 2, Selective Non-Electrophilic Thrombin | gi\|11514706 |
| Inhibitors | |
| Chain A, Crystal Structue Of Thrombin In Complex | |
| With | |
| A Potent P1 Heterocycle-Aryl Based Inhibitor | gi\|51247369 |
| Chain A, Crystal Structure Of Human Alpha-Thrombin | gi\|14278416 |
| Complexed With Bch-10556 And Exosite-Directed | |
| Peptide | |
| Chain A, Crystal Structure Of Human | gi\|11514051 |
| Alpha-ThrombinRwj-51438 Complex At 1.7 A | |
| Chain A, Crystal Structure Of Human Prethrombin-1 | gi\|311772208 |
| Chain A, Crystal Structure Of The Anticoagulant Slow | gi\|27065656 |
| Form Of Thrombin | |
| Chain A, Crystal Structure Of Thrombin In Complex | gi\|20150575 |
| With A Novel Bicyclic Lactam Inhibitor | |
| Chain A, Crystal Structure Of Thrombin In Complex | gi\|49259291 |
| With Compound 1 | |
| Chain A, Crystal Structure Of Thrombin In Complex | gi\|49259293 |
| With Compound 14b | |
| Chain A, Crystal Structure Of Thrombin In Complex | gi\|170292482 |
| With Inhibitor 15 | |
| Chain A, Design Of P1′ And P3′ Residues Of Trivalent | gi\|7766841 |
| Thrombin Inhibitors And Their Crystal Structures | |
| Chain A, Design Of P1′ And P3′ Residues Of Trivalent | gi\|7766843 |
| Thrombin Inhibitors And Their Crystal Structures | |
| Chain A, Nonbasic Thrombin Inhibitor Complex | gi\|67464458 |
| Chain A, Staphylocoagulase-Prethrombin-2 Complex | gi\|37927039 |
| Chain A, Structure Of Thrombin Complexed With | gi\|11514212 |
| Selective Non-Electophilic Inhibitors Having | |
| Cyclohexyl | |
| Moieties At P1 | |
| Chain A, Structure Of Thrombin Complexed With | gi\|11514286 |
| Selective Non-Electrophilic Inhibitors Having | |
| Cyclohexyl Moieties At P1 | |
| Chain A, Thrombin And P2 Pyridine N-Oxide Inhibitor | gi\|67464365 |
| Complex Structure | |
| Chain A, Thrombin In Complex With An Azafluorenyl | gi\|71042452 |
| Inhibitor 23b | |
| Chain A, Thrombin In Complex With An | gi\|78101061 |
| Oxazolopyridine Inhibitor 2 | |
| Chain A, Thrombin In Complex With An | gi\|78101058 |
| Oxazolopyridine Inhibitor | |
| 21 | |
| Chain A, Thrombin In Complex With | gi\|118137345 |
| Inhibitor | |
| Chain A, Thrombin In Complex With Selective | gi\|34810004 |
| Macrocyclic Inhibitor | |
| Chain A, Thrombin In Complex With Selective | gi\|34810002 |
| Macrocyclic Inhibitor At | |
| 1.8a | |
| Chain B, 1.9 Angstrom Crytal Structure Of Wild-Type | gi\|83753983 |
| Human Thrombin In The Sodium Free | |
| State | |
| Chain B, 2-(2-Chloro-6-Fluorophenyl)acetamides As | gi\|197304957 |
| Potent Thrombin Inhibitors | |
| Chain B, 2.1 Angstrom Structure Of A Nonproductive | gi\|116666780 |
| Complex Between Antithrombin, Synthetic Heparin | |
| Mimetic Sr123781 And Two S195a Thrombin | |
| Molecules | |
| Chain B, 2.5a Structure Of Anticoagulant Thrombin | gi\|48425307 |
| Variant | |
| E217k | |
| Chain B, Active Site Thrombin Inhibitors | gi\|157880622 |
| Chain B, Active Site Thrombin Inhibitors | |
| Chain B, Active Site Thrombin Inhibitors | gi\|60593850 |
| Chain B, Crystal Structure Of Human Alpha Thrombin | gi\|6435757 |
| In | |
| Complex With 5-Amidinoindole-4-Benzylpiperidine | |
| Inhibitor | |
| Chain B, Crystal Structure Of Human Alpha Thrombin | gi\|10835400 |
| In | |
| Complex With Benzo[b]thiophene | |
| Inhibitor 1 | |
| Chain B, Crystal Structure Of Human Alpha Thrombin | gi\|10835397 |
| In | |
| Complex With Benzo[b]thiophene | |
| Inhibitor 2 | |
| Chain B, Crystal Structure Of Human Alpha Thrombin | gi\|10835391 |
| In | |
| Complex With Benzothiophene Inhibitor 4 | |
| Chain B, Crystal Structure Of Human Aplha-Thrombin | gi\|10835394 |
| In Complex With Benzo[b]thiophene Inhibitor 3 | |
| Chain B, Crystal Structure Of Human Thrombin Mutant | gi\|261825076 |
| N143p In E Form | |
| Chain B, Crystal Structure Of Human Thrombin Mutant | gi\|261825074 |
| N143p In E:na+ Form | |
| Chain B, Crystal Structure Of Human Thrombin Mutant | gi\|291191316 |
| S195a In Complex Extracellular Fragment Of Human | |
| Par1 | |
| Chain B, Crystal Structure Of Na-Free Thrombin In | gi\|256032633 |
| Complex With Thrombomodulin | |
| Chain B, Crystal Structure Of Native S195a Thrombin | gi\|15825721 |
| With An Unoccupied Active Site | |
| Chain B, Crystal Structure Of | gi\|307776409 |
| Prethombin-2FRAGMENT-2 Complex | |
| Chain B, Crystal Structure Of The Anticoagulant Slow | gi\|49258988 |
| Form Of Thrombin | |
| Chain B, Crystal Structure Of The Anticoagulant Slow | gi\|27065657 |
| Form Of Thrombin | |
| Chain B, Crystal Structure Of The Anticoagulant | gi\|51247717 |
| Thrombin Mutant W215aE217A BOUND TO PPACK | |
| Chain B, Crystal Structure Of The Complex Between | gi\|99031771 |
| Thrombin And The Central “e” Region Of Fibrin | |
| Chain B, Crystal Structure Of The Complex Of Platelet | gi\|34811264 |
| Receptor Gpib-Alpha And Human Alpha-Thrombin | |
| Chain B, Crystal Structure Of The Potent | gi\|51247713 |
| Anticoagulant | |
| Thrombin Mutant W215aE217A IN FREE FORM | |
| Chain B, Crystal Structure Of The Procoagulant Fast | gi\|49258984 |
| Form Of Thrombin | |
| Chain B, Crystal Structure Of The Slow Form Of | gi\|116667325 |
| Thrombin In A Self-Inhibited | |
| Conformation | |
| Chain B, Crystal Structure Of The Slow Form Of | gi\|163931173 |
| Thrombin In A Self_inhibited | |
| Conformation | |
| Chain B, Crystal Structure Of The Thrombin Mutant | gi\|66360194 |
| D221aD222K | |
| Chain B, Crystal Structure Of The Thrombin Mutant | gi\|67464370 |
| G193a Bound To Ppack | |
| Chain B, Crystal Structure Of The Thrombin Mutant | gi\|67464373 |
| G193p Bound To Ppack | |
| Chain B, Crystal Structure Of The W215aE217A | gi\|251836913 |
| MUTANT OF HUMAN Thrombin (Space Group | |
| P2(1)2(1)2(1)) | |
| Chain B, Crystal Structure Of Thrombin Bound To | gi\|56967145 |
| Heparin | |
| Chain B, Crystal Structure Of Thrombin Bound To The | gi\|164414856 |
| Extracellular Fragment Of Par1 | |
| Chain B, Crystal Structure Of Thrombin In Complex | gi\|116667879 |
| With Fibrinogen Gamma′ Peptide | |
| Chain B, Crystal Structure Of Thrombin In Complex | gi\|237823757 |
| With Inhibitor | |
| Chain B, Crystal Structure Of Thrombin In Complex | gi\|47168355 |
| With L-378,622 | |
| Chain B, Cyanofluorophenylacetamides As Orally | gi\|223673888 |
| Efficacious Thrombin Inhibitors | |
| Chain B, Fast Form Of Thrombin Mutant R(77a)a | gi\|49258977 |
| Bound To Ppack | |
| Chain B, Human Thrombin Chimera With Human | gi\|146387601 |
| Residues 184a, 186, 186a, 186b, 186c And 222 | |
| Replaced By Murine Thrombin | |
| Equivalents. | |
| Chain B, Human Thrombin Mutant C191a-C220a In | gi\|152149319 |
| Complex With The Inhibitor Ppack | |
| Chain B, Inhibitor-Free Human Thrombin Mutant | gi\|152149317 |
| C191a- | |
| C220a | |
| Chain B, Novel Covalent Active Site Thrombin | gi\|9257032 |
| Inhibitors | |
| Chain B, Novel Covalent Active Site Thrombin | gi\|9257029 |
| Inhibitors | |
| Chain B, Novel Covalent Active Site Thrombin | gi\|8569363 |
| Inhibitors | |
| Chain B, Novel Covalent Active Site Thrombin | gi\|9257022 |
| Inhibitors | |
| Chain B, Novel Covalent Thrombin Inhibitor From | gi\|3891598 |
| Plant | |
| Extract | |
| Chain B, Ser195ala Mutant Of Human Thrombin | gi\|6980506 |
| Complexed With Fibrinopeptide A (7-16) | |
| Chain B, Slow Form Of Thrombin Bound With Ppack | gi\|49258995 |
| Chain B, Staphylocoagulase-Thrombin Complex | gi\|37927032 |
| Chain B, Structure Of Human Alpha-Thrombin Y225f | gi\|4699868 |
| Mutant Bound To D-Phe-Pro-Arg-Chloromethylketone | |
| Chain B, Structure Of Human Alpha-Thrombin Y225i | gi\|6980889 |
| Mutant Bound To D-Phe-Pro-Arg-Chloromethylketone | |
| Chain B, Structure Of Human Alpha-Thrombin Y225p | gi\|4699865 |
| Mutant Bound To D-Phe-Pro-Arg-Chloromethylketone | |
| Chain B, Structure Of Human Thrombin With Residues | gi\|332639879 |
| 145-150 Of Murine Thrombin. | |
| Chain B, Structure Of Thrombin Bound To The | gi\|169404722 |
| Inhibitor | |
| Fm19 | |
| Chain B, Structure Of Thrombin In 400 Mm Potassium | gi\|73536044 |
| Chloride | |
| Chain B, Structure Of Thrombin Mutant Delta(146- | gi\|238828222 |
| 149e) | |
| In The Free For | |
| Chain B, Thrombin In Complex With | gi\|185177551 |
| Inhibitor | |
| Chain B, Thrombin In Complex With Natural Product | gi\|56965950 |
| Inhibitor Oscillarin | |
| Chain B, Thrombin Inhibitor Complex | gi\|18655494 |
| Chain B, Thrombin Inhibitor Complex | gi\|16975148 |
| Chain B, Thrombin Inhibitor Complex | gi\|16975151 |
| Chain B, Thrombin Inhibitor Complex | |
| Chain B, Thrombin Inhibitor Complex | gi\|18655491 |
| Chain B, Thrombin Inhibitor With A Rigid Tripeptidyl | gi\|4929904 |
| Aldehydes | |
| Chain B, Thrombin | gi\|112490129 |
| Inhibitors | |
| Chain B, Thrombin | gi\|112490124 |
| Inhibitors | |
| Chain B, Thrombin | gi\|112490120 |
| Inhibitors | |
| Chain B, Thrombin | gi\|112490111 |
| Inhibitors | |
| Chain B, Thrombin | gi\|112490107 |
| Inhibitors | |
| Chain B, Thrombin | gi\|112490115 |
| Inhibitors | |
| Chain B, Thrombin Inhibitors With Rigid Tripeptidyl | gi\|4929962 |
| Aldehydes | |
| Chain B, Thrombin Inhibitors With Rigid Tripeptidyl | gi\|4929910 |
| Aldehydes | |
| Chain B, Thrombin-Hirugen Binary Complex At 1.26a | gi\|158430867 |
| Resolution | |
| Chain B, Thrombin-Hirugen-Gw420128 Ternary | gi\|158430873 |
| Complex At 1.39a | |
| Resolution | |
| Chain B, Thrombin-Hirugen-Gw473178 Ternary | gi\|158430870 |
| Complex At 1.32a | |
| Resolution | |
| Chain B, Thrombin-Hirugen-L405,426 | gi\|47168361 |
| Chain B, Thrombin-Hirugen_I-378,650 | gi\|47168358 |
| Chain B, X-Ray Structure Of The Complex Of Human | gi\|3891536 |
| Alpha Thrombin With The Inhibitor Sdz 229-357 | |
| Chain C, Antithrombin-Anhydrothrombin-Heparin | gi\|52695557 |
| Ternary Complex Structure | |
| Chain C, Crystal Structure Of The Complex Of Platelet | gi\|34811275 |
| Receptor Gpib-Alpha And Alpha-Thrombin At 2.6a | |
| Chain C, Crystallographic Structure Of Human | gi\|809212 |
| Gamma-Thrombin | |
| Chain D, 1.9 Angstrom Crytal Structure Of Wild-Type | gi\|83753985 |
| Human Thrombin In The Sodium Free | |
| State | |
| Chain D, 2.1 Angstrom Structure Of A Nonproductive | gi\|116666782 |
| Complex Between Antithrombin, Synthetic Heparin | |
| Mimetic Sr123781 And Two S195a Thrombin | |
| Molecules | |
| Chain D, 2.5a Structure Of Anticoagulant Thrombin | gi\|48425309 |
| Variant | |
| E217k | |
| Chain D, Crystal Structure Of Na-Free Thrombin In | gi\|256032635 |
| Complex With Thrombomodulin | |
| Chain D, Crystal Structure Of Native S195a Thrombin | gi\|15825723 |
| With An Unoccupied Active Site | |
| Chain D, Crystal Structure Of The Potent | gi\|51247715 |
| Anticoagulant | |
| Thrombin Mutant W215aE217A IN FREE FORM | |
| Chain D, Crystal Structure Of Thrombin Bound To | gi\|56967147 |
| Heparin | |
| Chain D, Crystal Structure Of Thrombin In Complex | gi\|116667881 |
| With Fibrinogen Gamma′ Peptide | |
| Chain D, Human Thrombin Hirugen Inhibitor Complex. | gi\|149242370 |
| Chain D, Human Thrombin Hirugen Inhibitor Complex. | gi\|149242365 |
| Chain D, Human Thrombin Hirugen Inhibitor Complex. | gi\|149242359 |
| Chain D, Novel Covalent Thrombin Inhibitor From | gi\|3891600 |
| Plant | |
| Extract | |
| Chain D, Staphylocoagulase-Prethrombin-2 Complex | gi\|37927041 |
| Chain D, Structure Of Thrombin In 400 Mm Potassium | gi\|73536046 |
| Chloride | |
| Chain E, Crystal Structure Of Human Thrombin Mutant | gi\|291191319 |
| S195a In Complex Extracellular Fragment Of Human | |
| Par1 | |
| Chain E, Crystal Structure Of Human Thrombin Mutant | gi\|251837025 |
| W215aE217A IN Complex With The Extracellular | |
| Fragment Of Human Par1 | |
| Chain E, Crystal Structure Of The Anticoagulant Slow | gi\|49258990 |
| Form Of Thrombin | |
| Chain E, Crystal Structure Of The Complex Between | gi\|99031773 |
| Thrombin And The Central “e” Region Of Fibrin | |
| Chain E, Crystal Structure Of The Procoagulant Fast | gi\|49258986 |
| Form Of Thrombin | |
| Chain E, Crystal Structure Of Thrombin Bound To The | gi\|164414859 |
| Extracellular Fragment Of Par1 | |
| Chain E, Fast Form Of Thrombin Mutant R(77a)a | gi\|49258979 |
| Bound To Ppack | |
| Chain E, Slow Form Of Thrombin Bound With Ppack | gi\|49258997 |
| Chain E, The Isomorphous Structures Of | gi\|809450 |
| Prethrombin2, | |
| Hirugen-And Ppack-Thrombin: Changes | |
| Accompanying | |
| Activation And Exosite Binding To | |
| Thrombin | |
| Chain F, Crystal Structure Of Na-Free Thrombin In | gi\|256032637 |
| Complex With Thrombomodulin | |
| Chain F, Crystal Structure Of Native S195a Thrombin | gi\|15825725 |
| With An Unoccupied Active Site | |
| Chain F, Crystal Structure Of Thrombin Bound To | gi\|56967149 |
| Heparin | |
| Chain F, Staphylocoagulase-Thrombin Complex | gi\|37927035 |
| Chain H, 1.6 A Structure Of The Pci-Thrombin-Heparin | gi\|185177830 |
| Complex | |
| Chain H, 2.5a Crystal Structure Of The | gi\|52695710 |
| Antithrombin-Thrombin-Heparin Ternary Complex | |
| Chain H, A Novel Serine Protease Inhibition Motif | gi\|157877660 |
| Involving A Multi-Centered Short Hydrogen Bonding | |
| Network At The Active Site | |
| Chain H, A Novel Serine Protease Inhibition Motif | gi\|157877655 |
| Involving A Multi-Centered Short Hydrogen Bonding | |
| Network At The Active Site | |
| Chain H, A Novel Serine Protease Inhibition Motif | gi\|18655740 |
| Involving A Multi-Centered Short Hydrogen Bonding | |
| Network At The Active Site | |
| Chain H, A Novel Serine Protease Inhibition Motif | gi\|18655743 |
| Involving A Multi-Centered Short Hydrogen Bonding | |
| Network At The Active Site | |
| Chain H, Active Site Mimetic Inhibition Of Thrombin | gi\|999824 |
| Chain H, Alpha-Thrombin (Hirugen) Complexed With | gi\|1943459 |
| Na-(N,N-Dimethylcarbamoyl)-Alpha-Azalysine | |
| Chain H, Alpha-Thrombin Complex With Sulfated | gi\|109156904 |
| Hirudin (Residues 54-65) And L-Arginine Template | |
| Inhibitor Cs107 | |
| Chain H, Alpha-Thrombin Complexed With Hirugen | gi\|1942044 |
| Chain H, Alpha-Thrombin Complexed With Hirugen | gi\|3891955 |
| And | |
| Proflavin | |
| Chain H, Bisphenylic Thrombin Inhibitors | gi\|237823760 |
| Chain H, Bisphenylic Thrombin Inhibitors | gi\|226887660 |
| Chain H, Bovine Thrombin--Bm51.1011 Complex | gi\|2781295 |
| Chain H, Changes In Interactions In Complexes Of | gi\|640438 |
| Hirudin Derivatives And Human Alpha-Thrombin Due | |
| To Different Crystal Forms | |
| Chain H, Changes In Interactions In Complexes Of | gi\|640434 |
| Hirudin Derivatives And Human Alpha-Thrombin Due | |
| To Different Crystal Forms | |
| Chain H, Complex Of A Divalent Inhibitor With | gi\|1633538 |
| Thrombin | |
| Chain H, Complex Of Human Alpha-Thrombin With A | gi\|1421172 |
| 15mer Oligonucleotide Ggttggtgtggttgg (Based On | |
| Nmr | |
| Model Of | |
| Dna | |
| Chain H, Complex Of Human Alpha-Thrombin With A | gi\|1421184 |
| 15mer Oligonucleotide Ggttggtgtggttgg (Based On | |
| X-Ray Model Of Dna) | |
| Chain H, Complex Of Human Alpha-Thrombin With | gi\|3319062 |
| The | |
| Bifunctional Boronate Inhibitor Borolog1 | |
| Chain H, Complex Of Human Alpha-Thrombin With | gi\|3319065 |
| The | |
| Bifunctional Boronate Inhibitor Borolog2 | |
| Chain H, Complex Of Recombinant Human Thrombin | gi\|33357811 |
| With A Designed Fluorinated Inhibitor | |
| Chain H, Complex Of Recombinant Human Thrombin | gi\|157881486 |
| With A Designed Inhibitor | |
| Chain H, Complex Of Thrombin With And Inhibitor | gi\|2624617 |
| Containing A Novel P1 Moiety | |
| Chain H, Complex Of Thrombin With Designed | gi\|71041796 |
| Inhibitor | |
| 7165 | |
| Chain H, Complex Structure Of Human Thrombin With | gi\|18655576 |
| N-Methyl-Arginine Inhibitor | |
| Chain H, Crystal Structure Analysis Of A Novel | gi\|62738009 |
| Oxyguanidine Bound To Thrombin | |
| Chain H, Crystal Structure Analysis Of A Novel | gi\|62738006 |
| Oxyguanidine Bound To Thrombin | |
| Chain H, Crystal Structure Of An Rna Aptamer Bound | gi\|212374978 |
| To Human Thrombin | |
| Chain H, Crystal Structure Of Human Alpha-Thrombin | gi\|1421284 |
| Complexed With Hirugen And | |
| P-Amidinophenylpyruvate) At 1.6 Angstroms | |
| Resolution | |
| Chain H, Crystal Structure Of Human Alpha-Thrombin | gi\|149241911 |
| In Complex With Suramin | |
| Chain H, Crystal Structure Of Human Alpha-Thrombin | gi\|7246027 |
| Inhibited With Sel2711. | |
| Chain H, Crystal Structure Of Sulfo-Hirudin | gi\|159795118 |
| Complexed | |
| To Thrombin | |
| Chain H, Crystal Structure Of The Complex Of Human | gi\|494209 |
| Alpha-Thrombin And Non-Hydrolyzable Bifunctional | |
| Inhibitors, Hirutonin-2 And Hirutonin-6 | |
| Chain H, Crystal Structure Of The Complex Of Human | gi\|494206 |
| Alpha-Thrombin And Non-Hydrolyzable Bifunctional | |
| Inhibitors, Hirutonin-2 And Hirutonin-6 | |
| Chain H, Crystal Structure Of The Heparin Cofactor | gi\|23200175 |
| li-S195a Thrombin | |
| Complex | |
| Chain H, Crystal Structure Of Thrombin Bound To | gi\|56967151 |
| Heparin | |
| Chain H, Crystal Structure Of Thrombin Complexed | gi\|6435561 |
| With An Guanidine-Mimetic Inhibitor | |
| Chain H, Crystal Structures Of Thrombin With | gi\|3212360 |
| Thiazole-Containing Inhibitors: Probes Of | |
| The S1′ Binding Site | |
| Chain H, Crystallographic Analysis At 3.0-Angstroms | gi\|493995 |
| Resolution Of The Binding To Human Thrombin Of | |
| Four | |
| Active Site-Directed Inhibitors | |
| Chain H, Crystallographic Analysis At 3.0-Angstroms | gi\|493989 |
| Resolution Of The Binding To Human Thrombin Of | |
| Four | |
| Active Site-Directed Inhibitors | |
| Chain H, Crystallographic Analysis At 3.0-Angstroms | gi\|493992 |
| Resolution Of The Binding To Human Thrombin Of | |
| Four | |
| Active Site-Directed Inhibitors | |
| Chain H, Crystallographic Analysis At 3.0-Angstroms | gi\|493986 |
| Resolution Of The Binding To Human Thrombin Of | |
| Four | |
| Active Site-Directed Inhibitors | |
| Chain H, Crystallographic Structures Of Thrombin | gi\|576213 |
| Complexed With Thrombin Receptor | |
| Peptides: Existence Of Expected And | |
| Novel Binding Modes | |
| Chain H, Crystallographic Structures Of Thrombin | gi\|576216 |
| Complexed With Thrombin Receptor | |
| Peptides: Existence Of Expected And | |
| Novel Binding Modes | |
| Chain H, Crystallographic Structures Of Thrombin | gi\|576210 |
| Complexed With Thrombin Receptor | |
| Peptides: Existence Of Expected And | |
| Novel Binding Modes | |
| Chain H, Crystallographic Structures Of Thrombin | gi\|576204 |
| Complexed With Thrombin Receptor | |
| Peptides: Existence Of Expected And | |
| Novel Binding Modes | |
| Chain H, Crystallographic Structures Of Thrombin | gi\|576220 |
| Complexed With Thrombin Receptor | |
| Peptides: Existence Of Expected And | |
| Novel Binding Modes | |
| Chain H, Crystallographic Structures Of Thrombin | gi\|576207 |
| Complexed With Thrombin Receptor | |
| Peptides: Existence Of Expected And | |
| Novel Binding Modes | |
| Chain H, D-Phe-Pro-Arg-Type Thrombin Inhibitor | gi\|38492568 |
| Chain H, Design And Discovery Of Novel, Potent | gi\|118137398 |
| Thrombin Inhibitors With A Solubilizing Cationic | |
| P1-P2-Linker | |
| Chain H, Design And Discovery Of Novel, Potent | gi\|118137395 |
| Thrombin Inhibitors With A Solubilizing Cationic | |
| P1-P2-Linker | |
| Chain H, Design And Discovery Of Novel, Potent | gi\|118137392 |
| Thrombin Inhibitors With A Solubilizing Cationic | |
| P1-P2-Linker | |
| Chain H, Design Of Weakly Basic Thrombin Inhibitors | gi\|34810220 |
| Incorporating Novel P1 Binding Functions: Molecular | |
| And X-Ray Crystallographic Studies. | |
| Chain H, Dissecting And Designing Inhibitor Selectivity | gi\|55669528 |
| Determinants At The S1 Site Using An Artificial Ala190 | |
| Protease (Ala190 Upa) | |
| Chain H, Elaborate Manifold Of Short Hydrogen Bond | gi\|31615744 |
| Arrays Mediating Binding Of Active Site-Directed | |
| Serine | |
| Protease Inhibitors | |
| Chain H, Exploring Thrombin S1 Pocket | gi\|211939077 |
| Chain H, Exploring Thrombin S1 Pocket | gi\|210060871 |
| Chain H, Exploring Thrombin S1 Pocket | gi\|210060874 |
| Chain H, Exploring Thrombin S1-Pocket | gi\|218681562 |
| Chain H, Exploring Thrombin S3 Pocket | gi\|223365748 |
| Chain H, Exploring Thrombin S3 Pocket | gi\|220702239 |
| Chain H, Exploring Thrombin S3 Pocket | gi\|220702242 |
| Chain H, Exploring Thrombin S3 Pocket | gi\|223365743 |
| Chain H, Exploring Thrombin S3 Pocket | |
| Chain H, Exploring Thrombin S3 Pocket | gi\|223365740 |
| Chain H, Exploring Thrombin S3 Pocket | gi\|221046516 |
| Chain H, Hirunorms Are True Hirudin Mimetics. The | gi\|2914466 |
| Crystal Structure Of Human Alpha-Thrombin:hirunorm | |
| V Complex | |
| Chain H, Human Alpha-Thrombin Complexed With A | gi\|1065046 |
| Peptidyl Pyridinium Methyl Ketone Containing Bivalent | |
| Inhibitor | |
| Chain H, Human Alpha-Thrombin Complexed With | gi\|1943483 |
| Ac-(D)phe-Pro-Boroornithine-Oh | |
| Chain H, Human Alpha-Thrombin Complexed With | gi\|1943480 |
| Ac-(D)phe-Pro-Boro-Homolys-Oh | |
| Chain H, Human Alpha-Thrombin Complexed With | gi\|1943477 |
| Ac-(D)phe-Pro-Boro-N-Butyl-Amidino-Glycine-Oh | |
| Chain H, Human Alpha-Thrombin Complexed With | gi\|1943530 |
| Ac-(D)phe-Pro-Boroarg-Oh | |
| Chain H, Human Alpha-Thrombin Complexed With | gi\|1943474 |
| Ac-(D)phe-Pro-Borolys-Oh | |
| Chain H, Human Alpha-Thrombin Complexed With | gi\|1943091 |
| [s-(R,R)]-1- | |
| (Aminoiminomethyl)-N-[[1[n-[(2- | |
| Naphthalenylsulfonyl)-L- | |
| Seryl]-Pyrrolidinyl]methyl]-3-Piperidenecarboxamide | |
| (Bms-189090) | |
| Chain H, Human Alpha-Thrombin Complexed With | gi\|1943088 |
| [s-(R,R)]-4- | |
| [(Aminoiminomethyl)amino]-N-[[1-[3-Hydroxy-2-[(2- | |
| Naphthalenylsulfonyl)amino]-1- | |
| Oxopropyl]-2-Pyrrolidinyl] | |
| Methyl]butanamide | |
| (Bms-186282) | |
| Chain H, Human Alpha-Thrombin In Complex With | gi\|6435720 |
| Bivalent, Benzamidine-Based Synthetic Inhibitor | |
| Chain H, Human Alpha-Thrombin Inhibition By | gi\|2781263 |
| Cbz-Pro-Azalys-Onp | |
| Chain H, Human Alpha-Thrombin Inhibition By | gi\|2781260 |
| Eoc-D-Phe-Pro- | |
| Azalys-Onp | |
| Chain H, Human Alpha-Thrombin Ternary Complex | gi\|2624876 |
| With Exosite Inhibitor Hirugen And Active Site Inhibitor | |
| Phch2oco-D-Dpa-Pro-Boroval | |
| Chain H, Human Alpha-Thrombin Ternary Complex | gi\|2624871 |
| With The Exosite Inhibitor Hirugen And Active Site | |
| Inhibitor Phch2oco-D-Dpa-Pro-Borompg | |
| Chain H, Human Thrombin Complex With Hirudin | gi\|1942457 |
| Variant | |
| Chain H, Human Thrombin Complexed With A | gi\|38492571 |
| D-Phe-Pro-Arg-Type Inhibitor And A C-Terminal | |
| Hirudin | |
| Derived Exo-Site Inhibitor | |
| Chain H, Human Thrombin Complexed | gi\|118137385 |
| With | |
| Fragment-Based Small Molecules Occupying The S1 | |
| Pocket | |
| Chain H, Human Thrombin Complexed | gi\|118137382 |
| With | |
| Fragment-Based Small Molecules Occupying The S1 | |
| Pocket | |
| Chain H, Human Thrombin Complexed With Novel | gi\|3212496 |
| Synthetic Peptide Mimetic Inhibitor And Hirugen | |
| Chain H, Human Thrombin Complexed With Novel | gi\|3212372 |
| Synthetic Peptide Mimetic Inhibitor And Hirugen | |
| Chain H, Human Thrombin With Active Site N-Methyl- | gi\|3212619 |
| D | |
| Phenylalanyl-N- | |
| [5-(Aminoiminomethyl)amino]-1- | |
| {{benzothiazolyl)carbon | |
| yl] Butyl]-L-Prolinamide Trifluroacetate | |
| And | |
| Exosite-Hirugen | |
| Chain H, Human Thrombin-In Complex With Ub-Thr10 | gi\|163931190 |
| Chain H, Human Thrombin-In Complex With Ub-Thr11 | gi\|163931193 |
| Chain H, Human Thrombin-Inhibitor | gi\|21465766 |
| Complex | |
| Chain H, Human Thrombin-Inhibitor | gi\|21465763 |
| Complex | |
| Chain H, Interaction Of Factor Xiii Activation Peptide | gi\|12084432 |
| With Alpha-Thrombin: Crystal Structure Of The | |
| Enzyme-Substrate | |
| Complex | |
| Chain H, Knoble Inhibitor | gi\|208435756 |
| Chain H, Molecular Basis For The Inhibition Of Human | gi\|494655 |
| Alpha-Thrombin By The Macrocyclic Peptide | |
| Cyclotheonamide A | |
| Chain H, Novel Covalent Thrombin Inhibitor From | gi\|3891595 |
| Plant | |
| Extract | |
| Chain H, Novel Non-Covalent Thrombin Inhibitors | gi\|49258947 |
| Incorporating P1 4,5,6,7-Tetrahydrobenzothiazole | |
| Arginine Side Chain | |
| Mimetics | |
| Chain H, Orally Active Thrombin | gi\|114793812 |
| Inhibitors | |
| Chain H, Orally Active Thrombin | gi\|109157608 |
| Inhibitors | |
| Chain H, Orally Active Thrombin Inhibitors In Complex | gi\|119388980 |
| With Thrombin And An Exosite | |
| Decapeptide | |
| Chain H, Orally Active Thrombin Inhibitors In Complex | gi\|119388977 |
| With Thrombin Inh12 | |
| Chain H, Recruiting Zinc To Mediate Potent, Specific | gi\|157838367 |
| Inhibition Of Serine Proteases | |
| Chain H, Recruiting Zinc To Mediate Potent, Specific | gi\|157838361 |
| Inhibition Of Serine Proteases | |
| Chain H, Recruiting Zinc To Mediate Potent, Specific | gi\|157838364 |
| Inhibition Of Serine Proteases | |
| Chain H, Selectivity At S1, H2o Displacement, Upa, | gi\|157877720 |
| Tpa, Ser190ALA190 PROTEASE, | |
| STRUCTURE-Based | |
| Drug Design | |
| Chain H, Selectivity At S1, H2o Displacement, Upa, | gi\|20663562 |
| Tpa, Ser190ALA190 PROTEASE, | |
| STRUCTURE-Based | |
| Drug Design | |
| Chain H, Serine Protease | gi\|157878283 |
| Chain H, Specifity For Plasminogen Activator Inhibitor-1 | gi\|4139650 |
| Chain H, Structural Basis For Selectivity Of A Small | gi\|157838402 |
| Molecule, S1-Binding, Sub-Micromolar Inhibitor Of | |
| Urokinase Type Plasminogen Activator | |
| Chain H, Structural Basis For Selectivity Of A Small | gi\|157838394 |
| Molecule, S1-Binding, Sub-Micromolar Inhibitor Of | |
| Urokinase Type Plasminogen Activator | |
| Chain H, Structural Basis For Selectivity Of A Small | gi\|157838399 |
| Molecule, S1-Binding, Sub-Micromolar Inhibitor Of | |
| Urokinase Type Plasminogen Activator | |
| Chain H, Structure Of A Retro-Binding Peptide | gi\|1311348 |
| Inhibitor | |
| Complexed With Human Alpha-Thrombin | |
| Chain H, Structure Of The Hirugen And Hirulog 1 | gi\|494778 |
| Complexes Of Alpha-Thrombin | |
| Chain H, Structure Of The Hirugen And Hirulog 1 | gi\|494150 |
| Complexes Of Alpha-Thrombin | |
| Chain H, Structure Of The Hirulog 3-Thrombin | gi\|493795 |
| Complex | |
| And Nature Of The S′ Subsites Of Substrates And | |
| Inhibitors | |
| Chain H, Structure Of The Hirulog 3-Thrombin | gi\|493792 |
| Complex | |
| And Nature Of The S′ Subsites Of Substrates And | |
| Inhibitors | |
| Chain H, Structure Of Thrombin Inhibited | gi\|3318750 |
| By | |
| Aeruginosin298-A From A Blue-Green | |
| Alga | |
| Chain H, Structures Of The Noncovalent Complexes | gi\|494787 |
| Of | |
| Human And Bovine Prothrombin Fragment 2 With | |
| Human Ppack-Thrombin | |
| Chain H, Structures Of The Noncovalent Complexes | gi\|494783 |
| Of | |
| Human And Bovine Prothrombin Fragment 2 With | |
| Human Ppack-Thrombin | |
| Chain H, Structures Of Thrombin Complexes With A | gi\|494650 |
| Designed And A Natural Exosite Inhibitor | |
| Chain H, Structures Of Thrombin Complexes With A | gi\|494647 |
| Designed And A Natural Exosite Inhibitor | |
| Chain H, Ternary Complex Of An Orally Active | gi\|110590819 |
| Thrombin Inhibitor With Human | |
| Thrombin And A C-Terminal Hirudin | |
| Derived Exo-Sit | |
| Inhibitor | |
| Chain H, The Crystal Structure Of | gi\|5542573 |
| Alpha-Thrombin-Hirunorm Iv Complex | |
| Reveals A Novel | |
| Specificity Site Recognition Mode. | |
| Chain H, The Interaction Of Thrombin With Fibrinogen: | gi\|494028 |
| A Structural Basis For Its Specificity | |
| Chain H, The Isomorphous Structures Of | gi\|809456 |
| Prethrombin2, | |
| Hirugen-And Ppack-Thrombin: Changes | |
| Accompanying | |
| Activation And Exosite Binding To | |
| Thrombin | |
| Chain H, The Isomorphous Structures Of | gi\|809453 |
| Prethrombin2, | |
| Hirugen-And Ppack-Thrombin: Changes | |
| Accompanying | |
| Activation And Exosite Binding To | |
| Thrombin | |
| Chain H, The Refined 1.9 Angstroms Crystal Structure | gi\|494462 |
| Of Human Alpha-Thrombin: Interaction | |
| With | |
| D-Phe-Pro-Arg Chloromethylketone And Significance | |
| Of | |
| The Tyr-Pro-Pro-Trp Insertion Segment | |
| Chain H, The Refined Structure Of The | gi\|494908 |
| Hirudin-Thrombin Complex | |
| Chain H, The Structure Of A Complex Of Recombinant | gi\|494874 |
| Hirudin And Human Alpha-Thrombin | |
| Chain H, The Structure Of A Nonadecapeptide Of The | gi\|809132 |
| Fifth Egf Domain Of Thrombomodulin Complexed With | |
| Thrombin | |
| Chain H, The Structure Of Alpha-Thrombin Inhibited | gi\|576170 |
| By | |
| A 15-Mer Single-Stranded Dna Aptamer | |
| Chain H, Thrombin Complexed With A Beta-Mimetic | gi\|3318985 |
| Thiazole-Containing | |
| Inhibitor | |
| Chain H, Thrombin Complexed With Hirugen And A | gi\|3212333 |
| Beta-Strand Mimetic | |
| Inhibitor | |
| Chain H, Thrombin In Complex With | gi\|210060868 |
| Inhibitor | |
| Chain H, Thrombin In Complex With | gi\|145579364 |
| Inhibitor | |
| Chain H, Thrombin | gi\|261824819 |
| Inhibition | |
| Chain H, Thrombin | gi\|226887656 |
| Inhibition | |
| Chain H, Thrombin | gi\|226887692 |
| Inhibition | |
| Chain H, Thrombin | gi\|220702250 |
| Inhibition | |
| Chain H, Thrombin | gi\|218681577 |
| Inhibition | |
| Chain H, Thrombin | gi\|218681570 |
| Inhibition | |
| Chain H, Thrombin | gi\|220702247 |
| Inhibition | |
| Chain H, Thrombin | gi\|220702253 |
| Inhibition | |
| Chain H, Thrombin | gi\|256032574 |
| Inhibitor | |
| Chain H, Thrombin Inhibitor Complex | gi\|88191873 |
| Chain H, Thrombin Inhibitor Complex | gi\|88191870 |
| Chain H, Thrombin Inhibitor Complex | gi\|88191864 |
| Chain H, Thrombin Inhibitor Complex | gi\|88191867 |
| Chain H, Thrombin Inhibitor From | gi\|2981772 |
| Theonalla, | |
| Cyclotheanamide-Based Macrocyclic Tripeptide Motif | gi\|218681567 |
| Chain H, Thrombin | |
| Inibition | |
| Chain H, Thrombin With 3-Cycle No F | gi\|192988238 |
| Chain H, Thrombin With 3-Cycle With F | gi\|192988242 |
| Chain H, Thrombin-Methoxy | gi\|157881466 |
| Chain H, Thrombin-Methoxy2 | gi\|110590949 |
| Chain H, Thrombin:suramin Complex | gi\|163931175 |
| Chain H, Understanding Thrombin | gi\|241913173 |
| Inhibition | |
| Chain H, Understanding Thrombin | gi\|241913162 |
| Inhibition | |
| Chain H, X-Ray Crystal Structure Of | gi\|13399530 |
| Human | |
| Alpha-Thrombin With A Tripeptide Phosphonate | |
| Inhibitor. | |
| Chain H, X-Ray Crystal Structure Of Thrombin | gi\|88191876 |
| Inhibited | |
| By Synthetic Cyanopeptide Analogue Ra-1008 | |
| Chain H, X-Ray Crystal Structure Of Thrombin | gi\|88191879 |
| Inhibited | |
| By Synthetic Cyanopeptide Analogue Ra-1014 | |
| Chain H, X-Ray Structure Of The Human | gi\|13400013 |
| Alpha-Thrombin Complex | |
| With A Tripeptide | |
| Phosphonate Inhibitor. | |
| Chain K, Interaction Of Factor Xiii Activation Peptide | gi\|12084434 |
| With Alpha-Thrombin: Crystal Structure Of The | |
| Enzyme-Substrate | |
| Complex | |
| Chain K, The Structure Of A Nonadecapeptide Of The | gi\|809135 |
| Fifth Egf Domain Of Thrombomodulin Complexed With | |
| Thrombin | |
| Coagulation factor II (thrombin) [Homo sapiens] | gi\|30802115 |
| RecName: Full = Prothrombin; AltName: | gi\|135807 |
| Full = Coagulation factor II; Contains: | |
| RecName: Full = Activation peptide | |
| fragment 1; Contains: RecName: | |
| Full = Activation peptide fragment 2; | |
| Contains: RecName: Full = Thrombin light | |
| chain; | |
| Contains: RecName: Full = Thrombin | |
| heavy chain; Flags: | |
| Precursor | |
| coagulation factor II (thrombin) [Homo sapiens] | gi\|18653448 |
| coagulation factor II (thrombin), isoform CRA_a [Homo | gi\|119588383 |
| sapiens] | |
| coagulation factor II (thrombin), isoform CRA_b [Homo | gi\|119588384 |
| sapiens] | |
| coagulation factor II (thrombin), isoform CRA_c [Homo | gi\|119588385 |
| sapiens] | |
| coagulation factor II precursor variant [Homo sapiens] | gi\|62897113 |
| coagulation factor II precursor variant [Homo sapiens] | gi\|62897109 |
| prothrombin B-chain [Homo sapiens] | gi\|38018092 |
| prothrombin [Homo sapiens] | gi\|339641 |
| prothrombin [Homo sapiens] | gi\|38018090 |
| prothrombin [Homo sapiens] | gi\|67624831 |
| prothrombin preproprotein [Homo sapiens] | gi\|4503635 |
| unnamed protein product [Homo sapiens] | gi\|1335344 |
| unnamed protein product [Homo sapiens] | gi\|110623182 |
| unnamed protein product [Homo sapiens] | gi\|194383496 |
| unnamed protein product [Homo sapiens] | gi\|194373497 |
| unnamed protein product [Homo sapiens] | gi\|189066554 |

Peptide Information

| Calc. | Obsrv. | | | Start | End | |
|---|---|---|---|---|---|---|
| Mass | Mass | ±da | ±ppm | Seq. | Seq. | Sequence |
| 1220.6271 | 1220.6044 | −.0227 | −19 | 53 | 62 | NFTENDLL |
| | | | | | | VR |

| | |
|---|---|
| hypothetical protein FLJ12998, | gi\|119615827 |
| isoform CRA_c [Homo sapiens] | |

Protein Group

| | |
|---|---|
| MTHFSD protein [Homo sapiens] | gi\|219520051 |
| Methenyltetrahydrofolate synthetase domain containing | gi\|85566723 |
| [Homo sapiens] | |
| Methenyltetrahydrofolate synthetase domain containing | gi\|85566720 |
| [Homo sapiens] | |
| RecName: Full = Methenyltetrahydrofolate synthase | gi\|209572652 |
| domain-containing protein | |
| Unknown (protein for MGC: 177233) [Homo sapiens] | gi\|219520048 |
| hypothetical protein FLJ12998, isoform CRA_a [Homo | gi\|119615825 |
| sapiens] | |
| hypothetical protein FLJ12998, isoform CRA_a [Homo | gi\|119615824 |
| sapiens] | |
| hypothetical protein FLJ12998, isoform CRA_d [Homo | gi\|119615828 |
| sapiens] | |
| methenyltetrahydrofolate synthase domain-containing | gi\|226823319 |
| protein isoform 1 [Homo sapiens] | |
| methenyltetrahydrofolate synthase domain-containing | gi\|226823322 |
| protein isoform 2 [Homo sapiens] | |
| methenyltetrahydrofolate synthase domain-containing | gi\|116875838 |
| protein isoform 3 [Homo sapiens] | |
| methenyltetrahydrofolate synthase domain-containing | gi\|226823324 |
| protein isoform 4 [Homo sapiens] | |
| methenyltetrahydrofolate synthase domain-containing | gi\|226823326 |
| protein isoform 5 [Homo sapiens] | |
| unnamed protein product [Homo sapiens] | gi\|194387430 |
| unnamed protein product [Homo sapiens] | i\|10436065 |
| unnamed protein product [Homo sapiens] | gi\|10434804 |
| unnamed protein product [Homo sapiens] | gi\|40038302 |
| unnamed protein product [Homo sapiens] | gi\|40034820 |

Peptide Information

| Calc. | Obsrv. | | | Start | End | |
|---|---|---|---|---|---|---|
| Mass | Mass | ±da | ±ppm | Seq. | Seq. | Sequence |
| 6271 | 1220.6044 | −.0227 | −19 | 63 | 73 | NYSVPIGLDSR |

| | | | |
|---|---|---|---|
| | 40/B13 | | Instr./Gel |
| | | | Origin |
| | [1] Sample Project | | Instrument |
| | 20110503 | | Sample |
| | | | Name |
| Protein Name- | KH7 (SEQ ID NO. KH7) | Accession No. | |

| | |
|---|---|
| unnamed protein product [Homo sapiens] | gi\|1335098 |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4447 | .0048 | 6 | 349 | 355 | LHIMAGR |
| 828.4111 | 828.4194 | .0083 | 10 | 55 | 60 | SHKWDR |
| 1070.5742 | 1070.5754 | .0012 | 1 | 191 | 199 | GEVPPRYPR |
| 1141.5889 | 1141.5918 | .0029 | 3 | 310 | 320 | GGYTLVSGYPK |
| 1142.6317 | 1142.6127 | −.019 | −17 | 80 | 89 | QGHNSVFLIK |
| 1158.4707 | 1158.4729 | .0022 | 2 | 203 | 211 | DYFMPCPGR |
| 1158.4707 | 1158.4729 | .0022 | 2 | 203 | 211 | DYFMPCPGR |
| 1220.606 | 1220.6071 | .0011 | 1 | 69 | 79 | NFPSPVDAAFR |
| 1220.606 | 1220.6071 | .0011 | 1 | 69 | 79 | NFPSPVDAAFR |
| 1268.6747 | 1268.6755 | .0008 | 1 | 186 | 196 | FDPVRGEVPPR |
| 1268.6747 | 1268.6755 | .0008 | 1 | 186 | 196 | FDPVRGEVPPR |
| 1495.6787 | 1495.6732 | −.0055 | −4 | 175 | 185 | YYCFQGNQFLR |
| 1495.6787 | 1495.6732 | −.0055 | −4 | 175 | 185 | YYCFQGNQFLR |
| 1512.6722 | 1512.702 | .0298 | 20 | 200 | 211 | DVRDYFMPCPGR |
| 1528.6672 | 1528.6807 | .0135 | 9 | 200 | 211 | DVRDYFMPCPGR |
| 1528.6672 | 1528.6807 | .0135 | 9 | 200 | 211 | DVRDYFMPCPGR |
| 1712.7698 | 1712.7638 | −.006 | −4 | 129 | 143 | GECQAEGVLFFQGDR |
| 2364.1653 | 2364.1492 | −.0161 | −7 | 108 | 128 | LLQDEFPGIPSP |
| | | | | | | LDAAVE |
| | | | | | | CHR |
| 2364.1653 | 2364.1492 | −.0161 | −7 | 108 | 128 | LLQDEFPGIPSP |
| | | | | | | LDAAVE |
| | 2364.1492 | | | | | CHR |
| 2498.2344 | 2498.2361 | .0017 | 1 | 325 | 348 | EVGTPHGIILDSV |
| | | | | | | DAAFIC |
| | | | | | | PGSSR |
| 2498.2344 | 2498.2361 | .0017 | 1 | 325 | 348 | EVGTPHGIILDSV |
| | | | | | | DAAFIC |
| | | | | | | PGSSR |

| | |
|---|---|
| hemopexin, isoform CRA_b [Homo sapiens] | gi\|119589125 |

Protein Group

| | |
|---|---|
| RecName: Full = Hemopexin; AltName: | gi\|1708182 |
| Full = Beta-1B- | |
| glycoprotein; Flags: | |
| Precursor | |
| hemopexin [Homo sapiens] | gi\|184497 |
| hemopexin precursor [Homo sapiens] | gi\|11321561 |
| hemopexin precursor [Homo sapiens] | gi\|386789 |
| unnamed protein product [Homo sapiens] | gi\|189053897 |
| unnamed protein product [Homo | gi\|80747621 |
| sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4447 | .0048 | 6 | 372 | 378 | LHIMAGR |
| 828.4111 | 828.4194 | .0083 | 10 | 78 | 83 | SHKWDR |
| 1070.5742 | 1070.5754 | .0012 | 1 | 214 | 222 | GEVPPRYPR |
| 1141.5889 | 1141.5918 | .0029 | 3 | 333 | 343 | GGYTLVSGYPK |
| 1142.6317 | 1142.6127 | −.019 | −17 | 103 | 112 | QGHNSVFLIK |
| 1158.4707 | 1158.4729 | .0022 | 2 | 226 | 234 | DYFMPCPGR |
| 1158.4707 | 1158.4729 | .0022 | 2 | 226 | 234 | DYFMPCPGR |
| 1220.606 | 1220.6071 | .0011 | 1 | 92 | 102 | NFPSPVDAAFR |
| 1220.606 | 1220.6071 | .0011 | 1 | 92 | 102 | NFPSPVDAAFR |
| 1268.6747 | 1268.6755 | .0008 | 1 | 209 | 219 | FDPVRGEVPPR |
| 1268.6747 | 1268.6755 | .0008 | 1 | 209 | 219 | FDPVRGEVPPR |
| 1495.6787 | 1495.6732 | −.0055 | −4 | 198 | 208 | YYCFQGNQFLR |
| 1495.6787 | 1495.6732 | −.0055 | −4 | 198 | 208 | YYCFQGNQFLR |
| 1512.6722 | 1512.702 | .0298 | 20 | 223 | 234 | DVRDYFMPCPGR |
| 1528.6672 | 1528.6807 | .0135 | 9 | 223 | 234 | DVRDYFMPCPGR |
| 1528.6672 | 1528.6807 | .0135 | 9 | 223 | 234 | DVRDYFMPCPGR |
| 1712.7698 | 1712.7638 | −.006 | −4 | 152 | 166 | GECQAEGVLFFQGDR |
| 2364.1653 | 2364.1492 | −.0161 | −7 | 131 | 151 | LLQDEFPGIPSP |
| | | | | | | LDAAVE |
| | | | | | | CHR |
| 2364.1653 | 2364.1492 | −.0161 | −7 | 131 | 151 | LLQDEFPGIPSP |
| | | | | | | LDAAVE |
| | | | | | | CHR |
| 2498.2344 | 2498.2361 | .0017 | 1 | 348 | 371 | EVGTPHGIILDSV |
| | | | | | | DAAFIC |
| | | | | | | PGSSR |
| 2498.2344 | 2498.2361 | .0017 | 1 | 348 | 371 | EVGTPHGIILDSV |
| | | | | | | DAAFIC |
| | | | | | | PGSSR |

| | |
|---|---|
| HPX protein [Homo sapiens] | gi\|13529281 |

Peptide Information

| | | | | Start | End | |
|---|---|---|---|---|---|---|
| Calc. Mass | Obsrv. Mass | ±da | ±ppm | Seq. | Seq. | Sequence |
| 828.4111 | 828.4194 | .0083 | 10 | 78 | 83 | SHKWDR |
| 1070.5742 | 1070.5754 | .0012 | 1 | 214 | 222 | GEVPPRYPR |
| 1142.6317 | 1142.6127 | −.019 | −17 | 103 | 112 | QGHNSVFLIK |
| 1158.4707 | 1158.4729 | .0022 | 2 | 226 | 234 | DYFMPCPGR |
| 1158.4707 | 1158.4729 | .0022 | 2 | 226 | 234 | DYFMPCPGR |
| 1220.606 | 1220.6071 | .0011 | 1 | 92 | 102 | NFPSPVDAAFR |
| 1220.606 | 1220.6071 | .0011 | 1 | 92 | 102 | NFPSPVDAAFR |
| 1268.6747 | 1268.6755 | .0008 | 1 | 209 | 219 | FDPVRGEVPPR |
| 1268.6747 | 1268.6755 | .0008 | 1 | 209 | 219 | FDPVRGEVPPR |
| 1495.6787 | 1495.6732 | −.0055 | −4 | 198 | 208 | YYCFQGNQFLR |
| 1495.6787 | 1495.6732 | −.0055 | −4 | 198 | 208 | YYCFQGNQFLR |
| 1512.6722 | 1512.702 | .0298 | 20 | 223 | 234 | DVRDYFMPCPGR |
| 1528.6672 | 1528.6807 | .0135 | 9 | 223 | 234 | DVRDYFMPCPGR |
| 1528.6672 | 1528.6807 | .0135 | 9 | 223 | 234 | DVRDYFMPCPGR |
| 1712.7698 | 1712.7638 | −.006 | −4 | 152 | 166 | GECQAEGVLFFQGDR |
| 2364.1653 | 2364.1492 | −.0161 | −7 | 131 | 151 | LLQDEFPGIPSP |
| | | | | | | LDAAVE |
| | | | | | | CHR |
| 2364.1653 | 2364.1492 | −.0161 | −7 | 131 | 151 | LLQDEFPGIPSP |
| | | | | | | LDAAVE |
| | | | | | | CHR |

| | |
|---|---|
| hemopexin, isoform CRA_d [Homo sapiens] | gi\|119589127 |

Peptide Information

| Calc. Mass | Obsrv. Mass | ±da | ±ppm | Start Seq. | End Seq. | Sequence |
|---|---|---|---|---|---|---|
| 813.4399 | 813.4447 | .0048 | 6 | 301 | 307 | LHIMAGR |
| 828.4111 | 828.4194 | .0083 | 10 | 78 | 83 | SHKWDR |
| 1141.5889 | 1141.5918 | .0029 | 3 | 262 | 272 | GGYTLVSGYPK |
| 1142.6317 | 1142.6127 | −.019 | −17 | 103 | 112 | QGHNSVFLIK |
| 1220.606 | 1220.6071 | .0011 | 1 | 92 | 102 | NFPSPVDAAFR |
| 1220.606 | 1220.6071 | .0011 | 1 | 92 | 102 | NFPSPVDAAFR |
| 2364.1653 | 2364.1492 | −.0161 | −7 | 131 | 151 | LLQDEFPGIPSPL |
| | | | | | | DAAVE |
| | | | | | | CHR |
| 2364.1653 | 2364.1492 | −.0161 | −7 | 131 | 151 | LLQDEFPGIPSPL |
| | | | | | | DAAVE |
| | | | | | | CHR |
| 2498.2344 | 2498.2361 | .0017 | 1 | 277 | 300 | EVGTPHGIILDSV |
| | | | | | | DAAFIC |
| | | | | | | PGSSR |
| 2498.2344 | 2498.2361 | .0017 | 1 | 277 | 300 | EVGTPHGIILDSV |
| | | | | | | DAAFIC |
| | | | | | | PGSSR |

| | |
|---|---|
| hemopexin, isoform CRA_a [Homo sapiens] | gi\|119589124 |

Peptide Information

| Calc. | Obsrv. | | | Start | End | |
|---|---|---|---|---|---|---|
| Mass | Mass | ±da | ±ppm | Seq. | Seq. | Sequence |
| 813.4399 | 813.4447 | .0048 | 6 | 118 | 124 | LHIMAGR |
| 1141.5889 | 1141.5918 | .0029 | 3 | 79 | 89 | GGYTLVSG |
| | | | | | | YPK |
| 2498.2344 | 2498.2361 | .0017 | 1 | 94 | 117 | EVGTPHGI |
| | | | | | | ILDSV |
| | | | | | | DAAFIC |
| | | | | | | PGSSR |
| 2498.2344 | 2498.2361 | .0017 | 1 | 94 | 117 | EVGTPHGI |
| | | | | | | ILDSV |
| | | | | | | DAAFIC |
| | | | | | | PGSSR |

| | |
|---|---|
| hemopexin | gi\|226337 |

Peptide Information

| Calc. | Obsrv. | | | Start | End | |
|---|---|---|---|---|---|---|
| Mass | Mass | ±da | ±ppm | Seq. | Seq. | Sequence |
| 1141.5889 | 1141.5918 | .0029 | 3 | 78 | 88 | GGYTLVSG |
| | | | | | | YPK |
| 2498.2344 | 2498.2361 | .0017 | 1 | 93 | 116 | EVGTPHGI |
| | | | | | | ILDSV |
| | | | | | | DAAFIC |
| | | | | | | PGSSR |
| 2498.2344 | 2498.2361 | .0017 | 1 | 93 | 116 | EVGTPHGI |
| | | | | | | ILDSV |
| | | | | | | DAAFIC |
| | | | | | | PGSSR |

| | |
|---|---|
| hemopexin, isoform CRA_c | gi\|119589126 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 813.4399 | 813.4447 | .0048 | 6 | 170 | 176 | LHIMAGR |
| 1141.5889 | 1141.5918 | .0029 | 3 | 131 | 141 | GGYTLVSG |
| | | | | | | YPK |
| 2498.2344 | 2498.2361 | .0017 | 1 | 146 | 169 | EVGTPHGI |
| | | | | | | ILDSV |
| | | | | | | DAAFIC |
| | | | | | | PGSSR |
| 2498.2344 | 2498.2361 | .0017 | 1 | 146 | 169 | EVGTPHGI |
| | | | | | | ILDSV |
| | | | | | | DAAFIC |
| | | | | | | PGSSR |

| | |
|---|---|
| unnamed protein product | gi\|221044726 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 828.4111 | 828.4194 | .0083 | 10 | 78 | 83 | SHKWDR |
| 1142.6317 | 1142.6127 | −.019 | −17 | 103 | 112 | QGHNSVF |
| | | | | | | LIK |
| 1220.606 | 1220.6071 | .0011 | 1 | 92 | 102 | NFPSPVDA |
| | | | | | | AFR |
| 1220.606 | 1220.6071 | .0011 | 1 | 92 | 102 | NFPSPVDA |
| | | | | | | AFR |

| | |
|---|---|
| plectin 8 [Homo sapiens] | gi\|40849940 |

Protein Group

| | |
|---|---|
| plectin isoform 1b [Homo sapiens] | gi\|41322919 |

Peptide Information

| | | | | Start | End | |
|---|---|---|---|---|---|---|
| Calc. Mass | Obsrv. Mass | ±da | ±ppm | Seq. | Seq. | Sequence |
| 819.4393 | 819.3938 | −.0455 | −56 | 4368 | 4375 | MSAAQALK |
| 828.3668 | 828.4194 | .0526 | 63 | 748 | 753 | KYSCDR |
| 860.4108 | 860.4073 | −.0035 | −4 | 2110 | 2116 | QVEEAER |
| 860.422 | 860.4073 | −.0147 | −17 | 1623 | 1629 | AREEAER |
| 930.489 | 930.4886 | −.0004 | 0 | 2450 | 2457 | EAIAELER |
| 956.4506 | 956.4131 | −.0375 | −39 | 656 | 663 | ESYSALMR |
| 1036.5786 | 1036.5348 | −.0438 | −42 | 486 | 493 | TEYNLRLK |
| 1037.6255 | 1037.5444 | −.0811 | −78 | 51 | 58 | WVNKHLIK |
| 1068.5143 | 1068.5271 | .0128 | 12 | 171 | 179 | MVEGYQGLR |
| 1084.5455 | 1084.5914 | .0459 | 42 | 655 | 663 | KESYSALMR |
| 1107.5729 | 1107.5511 | −.0218 | −20 | 2970 | 2978 | GRLCFEG |
| | | | | | | LR |
| 1128.6121 | 1128.5707 | −.0414 | −37 | 1199 | 1207 | QRELEQL |
| | | | | | | GR |
| 1142.6528 | 1142.6127 | −.0401 | −35 | 1450 | 1458 | IEEEIRVVR |
| 1161.6296 | 1161.6194 | −.0102 | −9 | 4339 | 4348 | GLVDKIMV |
| | | | | | | DR |
| 1177.5994 | 1177.6208 | .0214 | 18 | 2548 | 2557 | LVASMEE |
| | | | | | | ARR |
| 1193.5765 | 1193.6279 | .0514 | 43 | 982 | 990 | CQRCISELK |
| 1220.6344 | 1220.6071 | −.0273 | −22 | 3993 | 4003 | MGIVGPEF |
| | | | | | | KDK |
| 1220.6555 | 1220.6071 | −.0484 | −40 | 2235 | 2244 | VQMEELS |
| | | | | | | KLK |
| 1268.6191 | 1268.6755 | .0564 | 44 | 2265 | 2274 | FLQEEAEKMK |
| 1268.6191 | 1268.6755 | .0564 | 44 | 2265 | 2274 | FLQEEAEKMK |
| 1429.8236 | 1429.7775 | −.0461 | −32 | 286 | 296 | ELVLLLLQWMR |
| 1445.8112 | 1445.7743 | −.0369 | −26 | 100 | 111 | LQNVQIALDYLR |
| 1463.858 | 1463.9216 | .0636 | 43 | 1291 | 1304 | AQLEPVASPAKK |
| | | | | | | PK |
| 1477.7316 | 1477.7634 | .0318 | 22 | 3900 | 3912 | SQVMDEATALQLR |
| 1495.7792 | 1495.6732 | −.106 | −71 | 2903 | 2916 | DVFEKATVSAPF |
| | | | | | | GK |
| 1495.7792 | 1495.6732 | −.106 | −71 | 2903 | 2916 | DVFEKATVSAPF |
| | | | | | | GK |
| 1510.702 | 1510.6783 | −.0237 | −16 | 4531 | 4547 | YASGSSASLGGP |
| | | | | | | ESAVA |
| 1512.6925 | 1512.702 | .0095 | 6 | 3832 | 3844 | DTHDQLSEPSEVR |
| 1528.739 | 1528.6807 | −.0583 | −38 | 1214 | 1227 | ESADPLGAWLQ |
| | | | | | | DAR |
| 1528.739 | 1528.6807 | −.0583 | −38 | 1214 | 1227 | ESADPLGAWLQ |
| | | | | | | DAR |
| 1544.785 | 1544.7278 | −.0572 | −37 | 1392 | 1403 | ELQQRMQEEVVR |
| 1566.7759 | 1566.7615 | −.0144 | −9 | 2888 | 2902 | AAKGGELVYTDS |
| | | | | | | EAR |
| 1613.8745 | 1613.7207 | −.1538 | −95 | 3133 | 3148 | LLDAQLSTGGIV |
| | | | | | | DPSK |
| 1667.885 | 1667.7976 | −.0874 | −52 | 1114 | 1129 | EAQAVPATLPEL |
| | | | | | | EATK |
| 1684.8402 | 1684.8882 | .048 | 28 | 1214 | 1228 | ESADPLGAWLQ |
| | | | | | | DARR |
| 1684.8402 | 1684.8882 | .048 | 28 | 1214 | 1228 | ESADPLGAWLQ |
| | | | | | | DARR |
| 1726.9083 | 1726.808 | −.1003 | −58 | 3866 | 3881 | RDDGTGQLLLPL |
| | | | | | | SDAR |
| 1869.9777 | 1869.87 | −.1077 | −58 | 2275 | 2292 | QVAEEAARLSVA |
| | | | | | | AQEAAR |
| 1883.887 | 1883.8921 | .0051 | 3 | 3549 | 3564 | AEVVETTQVYTE |
| | | | | | | EETR |
| 1915.947 | 1915.8633 | −.0837 | −44 | 3773 | 3788 | DPYTEQTISLFQA |
| | | | | | | MKK |
| 2480.3396 | 2480.2053 | −.1343 | −54 | 3647 | 3667 | LTAEDLFEARIISL |
| | | | | | | ETYNLLR |
| 2480.3396 | 2480.2053 | −.1343 | −54 | 3647 | 3667 | LTAEDLFEARIISL |
| | | | | | | ETYNLL R |

| | |
|---|---|
| plectin 11 [Homo sapiens] | gi\|40849944 |

Protein Group

| | |
|---|---|
| plectin isoform 1a [Homo sapiens] | gi\|41322923 |

Peptide Information

| | Obsrv. | | | Start | End | |
|---|---|---|---|---|---|---|
| Calc. Mass | Mass | ±da | ±ppm | Seq. | Seq. | Sequence |
| 819.4393 | 819.3938 | −.0455 | −56 | 4368 | 4375 | MSAAQALK |
| 828.3668 | 828.4194 | .0526 | 63 | 748 | 753 | KYSCDR |
| 860.4108 | 860.4073 | −.0035 | −4 | 2110 | 2116 | QVEEAER |
| 860.422 | 860.4073 | −.0147 | −17 | 1623 | 1629 | AREEAER |
| | 860.4073 | | | | | |
| 956.4506 | 956.4131 | −.0375 | −39 | 656 | 663 | ESYSALMR |
| 1036.5786 | 1036.5348 | −.0438 | −42 | 486 | 493 | TEYNLRLK |
| 1037.6255 | 1037.5444 | −.0811 | −78 | 51 | 58 | WVNKHLIK |
| 1068.5143 | 1068.5271 | .0128 | 12 | 171 | 179 | MVEGYQGLR |
| 1084.5455 | 1084.5914 | .0459 | 42 | 655 | 663 | KESYSALMR |
| 1107.5729 | 1107.5511 | −.0218 | −20 | 2970 | 2978 | GRLCFEGLR |
| 1128.6121 | 1128.5707 | −.0414 | −37 | 1199 | 1207 | QRELEQLGR |
| 1142.6528 | 1142.6127 | −.0401 | −35 | 1450 | 1458 | IEEEIRVVR |
| 1161.6296 | 1161.6194 | −.0102 | −9 | 4339 | 4348 | GLVDKIMVDR |
| 1177.5994 | 1177.6208 | .0214 | 18 | 2548 | 2557 | LVASMEEARR |
| 1193.5765 | 1193.6279 | .0514 | 43 | 982 | 990 | CQRCISELK |
| 1220.6344 | 1220.6071 | −.0273 | −22 | 3993 | 4003 | MGIVGPEFKDK |
| 1220.6555 | 1220.6071 | −.0484 | −40 | 2235 | 2244 | VQMEELSKLK |
| 1268.6191 | 1268.6755 | .0564 | 44 | 2265 | 2274 | FLQEEAEKMK |
| 1268.6191 | 1268.6755 | .0564 | 44 | 2265 | 2274 | FLQEEAEKMK |
| 1429.8236 | 1429.7775 | −.0461 | −32 | 286 | 296 | ELVLLLLQWMR |
| 1445.8112 | 1445.7743 | −.0369 | −26 | 100 | 111 | LQNVQIALDYLR |
| 1463.858 | 1463.9216 | .0636 | 43 | 1291 | 1304 | AQLEPVASPAKK |
| | | | | | | PK |
| 1477.7316 | 1477.7634 | .0318 | 22 | 3900 | 3912 | SQVMDEATALQLR |
| 1495.7792 | 1495.6732 | −.106 | −71 | 2903 | 2916 | DVFEKATVSAPF |
| | | | | | | GK |
| 1495.7792 | 1495.6732 | −.106 | −71 | 2903 | 2916 | DVFEKATVSAPF |
| | | | | | | GK |
| 1510.702 | 1510.6783 | −.0237 | −16 | 4531 | 4547 | YASGSSASLGGP |
| | | | | | | ESAVA |
| 1512.6925 | 1512.702 | .0095 | 6 | 3832 | 3844 | DTHDQLSEPSEVR |
| 1528.739 | 1528.6807 | −.0583 | −38 | 1214 | 1227 | ESADPLGAWLQD |
| | | | | | | AR |
| 1528.739 | 1528.6807 | −.0583 | −38 | 1214 | 1227 | ESADPLGAWLQD |
| | | | | | | AR |
| 1544.785 | 1544.7278 | −.0572 | −37 | 1392 | 1403 | ELQQRMQEEVVR |
| 1566.7759 | 1566.7615 | −.0144 | −9 | 2888 | 2902 | AAKGGELVYTDS |
| | | | | | | EAR |
| 1613.8745 | 1613.7207 | −.1538 | −95 | 3133 | 3148 | LLDAQLSTGGIVD |
| | | | | | | PSK |
| 1667.885 | 1667.7976 | −.0874 | −52 | 1114 | 1129 | EAQAVPATLPELE |
| | | | | | | ATK |
| 1684.8402 | 1684.8882 | .048 | 28 | 1214 | 1228 | ESADPLGAWLQD |
| | | | | | | ARR |
| 1684.8402 | 1684.8882 | .048 | 28 | 1214 | 1228 | ESADPLGAWLQD |
| | | | | | | ARR |
| 1726.9083 | 1726.808 | −.1003 | −58 | 3866 | 3881 | RDDGTGQLLLPL |
| | | | | | | SDAR |
| 1869.9777 | 1869.87 | −.1077 | −58 | 2275 | 2292 | QVAEEAARLSVA |
| | | | | | | AQEAAR |
| 1883.887 | 1883.8921 | .0051 | 3 | 3549 | 3564 | AEVVETTQVYTEE |
| | | | | | | ETR |
| 1915.947 | 1915.8633 | −.0837 | −44 | 3773 | 3788 | DPYTEQTISLFQA |
| | | | | | | MKK |
| 2480.3396 | 2480.2053 | −.1343 | −54 | 3647 | 3667 | LTAEDLFEARIISL |
| | | | | | | ETYNLLR |
| 2480.3396 | 2480.2053 | −.1343 | −54 | 3647 | 3667 | LTAEDLFEARIISL |
| | | | | | | ETYNLL R |

| | |
|---|---|
| plectin 3 [Homo sapiens] | gi\|40849934 |

Protein Group

| | |
|---|---|
| plectin isoform 1e [Homo sapiens] | gi\|41322908 |

Peptide Information

| | | | | Start | End | |
|---|---|---|---|---|---|---|
| Calc. Mass | Obsrv. Mass | ±da | ±ppm | Seq. | Seq. | Sequence |
| 819.4393 | 819.3938 | −.0455 | −56 | 4346 | 4353 | MSAAQALK |
| 828.3668 | 828.4194 | .0526 | 63 | 726 | 731 | KYSCDR |
| 860.4108 | 860.4073 | −.0035 | −4 | 2088 | 2094 | QVEEAER |
| 860.422 | 860.4073 | −.0147 | −17 | 1601 | 1607 | AREEAER |
| 930.489 | 930.4886 | −.0004 | 0 | 2428 | 2435 | EAIAELER |
| 956.4506 | 956.4131 | −.0375 | −39 | 634 | 641 | ESYSALMR |
| 1036.5786 | 1036.5348 | −.0438 | −42 | 464 | 471 | TEYNLRLK |
| 1037.6255 | 1037.5444 | −.0811 | −78 | 29 | 36 | WVNKHLIK |
| 1068.5143 | 1068.5271 | .0128 | 12 | 149 | 157 | MVEGYQGLR |
| 1084.5455 | 1084.5914 | .0459 | 42 | 633 | 641 | KESYSALMR |
| 1107.5729 | 1107.5511 | −.0218 | −20 | 2948 | 2956 | GRLCFEGLR |
| 1128.6121 | 1128.5707 | −.0414 | −37 | 1177 | 1185 | QRELEQLGR |
| 1142.6528 | 1142.6127 | −.0401 | −35 | 1428 | 1436 | IEEEIRVVR |
| 1161.6296 | 1161.6194 | −.0102 | −9 | 4317 | 4326 | GLVDKIMVDR |
| 1177.5994 | 1177.6208 | .0214 | 18 | 2526 | 2535 | LVASMEEARR |
| 1193.5765 | 1193.6279 | .0514 | 43 | 960 | 968 | CQRCISELK |
| 1220.6344 | 1220.6071 | −.0273 | −22 | 3971 | 3981 | MGIVGPEFKDK |
| 1220.6555 | 1220.6071 | −.0484 | −40 | 2213 | 2222 | VQMEELSKLK |
| 1268.6191 | 1268.6755 | .0564 | 44 | 2243 | 2252 | FLQEEAEKMK |
| 1268.6191 | 1268.6755 | .0564 | 44 | 2243 | 2252 | FLQEEAEKMK |
| 1429.8236 | 1429.7775 | −.0461 | −32 | 264 | 274 | ELVLLLLQWMR |
| 1445.8112 | 1445.7743 | −.0369 | −26 | 78 | 89 | LQNVQIALDYLR |
| 1463.858 | 1463.9216 | .0636 | 43 | 1269 | 1282 | AQLEPVASPAKK |
| | | | | | | PK |
| 1477.7316 | 1477.7634 | .0318 | 22 | 3878 | 3890 | SQVMDEATALQLR |
| 1495.7792 | 1495.6732 | −.106 | −71 | 2881 | 2894 | DVFEKATVSAPF |
| | | | | | | GK |
| 1495.7792 | 1495.6732 | −.106 | −71 | 2881 | 2894 | DVFEKATVSAPF |
| | 1495.6732 | | | | | GK |
| 1510.702 | 1510.6783 | −.0237 | −16 | 4509 | 4525 | YASGSSASLGGP |
| | | | | | | ESAVA |
| 1512.6925 | 1512.702 | .0095 | 6 | 3810 | 3822 | DTHDQLSEPSEVR |
| 1528.739 | 1528.6807 | −.0583 | −38 | 1192 | 1205 | ESADPLGAWLQD |
| | | | | | | AR |
| 1528.739 | 1528.6807 | −.0583 | −38 | 1192 | 1205 | ESADPLGAWLQD |
| | | | | | | AR |
| 1544.785 | 1544.7278 | −.0572 | −37 | 1370 | 1381 | ELQQRMQEEVVR |
| 1566.7759 | 1566.7615 | −.0144 | −9 | 2866 | 2880 | AAKGGELVYTDS |
| | | | | | | EAR |
| 1613.8745 | 1613.7207 | −.1538 | −95 | 3111 | 3126 | LLDAQLSTGGIV |
| | | | | | | DPSK |
| 1667.885 | 1667.7976 | −.0874 | −52 | 1092 | 1107 | EAQAVPATLPEL |
| | | | | | | EATK |
| 1684.8402 | 1684.8882 | .048 | 28 | 1192 | 1206 | ESADPLGAWLQ |
| | | | | | | DARR |
| 1684.8402 | 1684.8882 | .048 | 28 | 1192 | 1206 | ESADPLGAWLQ |
| | | | | | | DARR |
| 1726.9083 | 1726.808 | −.1003 | −58 | 3844 | 3859 | RDDGTGQLLLPL |
| | | | | | | SDAR |
| 1869.9777 | 1869.87 | −.1077 | −58 | 2253 | 2270 | QVAEEAARLSVA |
| | | | | | | AQEAAR |
| 1883.887 | 1883.8921 | .0051 | 3 | 3527 | 3542 | AEVVETTQVYTE |
| | | | | | | EETR |
| 1915.947 | 1915.8633 | −.0837 | −44 | 3751 | 3766 | DPYTEQTISLFQA |
| | | | | | | MKK |
| 2480.3396 | 2480.2053 | −.1343 | −54 | 3625 | 3645 | LTAEDLFEARIISL |
| | | | | | | ETYNLLR |
| 2480.3396 | 2480.2053 | −.1343 | −54 | 3625 | 3645 | LTAEDLFEARIISL |
| | | | | | | ETYNLL R |

| | |
|---|---|
| plectin 2 [Homo sapiens] | gi\|40849932 |

Protein Group

| | |
|---|---|
| plectin isoform 1f [Homo sapiens] | gi\|41322912 |

Peptide Information

| | | | | Start | | |
|---|---|---|---|---|---|---|
| Calc. Mass | Obsrv. Mass | ±da | ±ppm | Seq. | End Seq. | Sequence |
| 819.4393 | 819.3938 | −.0455 | −56 | 4354 | 4361 | MSAAQALK |
| 828.3668 | 828.4194 | .0526 | 63 | 734 | 739 | KYSCDR |
| 860.4108 | 860.4073 | −.0035 | −4 | 2096 | 2102 | QVEEAER |
| 860.422 | 860.4073 | −.0147 | −17 | 1609 | 1615 | AREEAER |
| 930.489 | 930.4886 | −.0004 | 0 | 2436 | 2443 | EAIAELER |
| 956.4506 | 956.4131 | −.0375 | −39 | 642 | 649 | ESYSALMR |
| 1036.5786 | 1036.5348 | −.0438 | −42 | 472 | 479 | TEYNLRLK |
| 1037.6255 | 1037.5444 | −.0811 | −78 | 37 | 44 | WVNKHLIK |
| 1068.5143 | 1068.5271 | .0128 | 12 | 157 | 165 | MVEGYQGLR |
| 1084.5455 | 1084.5914 | .0459 | 42 | 641 | 649 | KESYSALMR |
| 1107.5729 | 1107.5511 | −.0218 | −20 | 2956 | 2964 | GRLCFEGLR |
| 1128.6121 | 1128.5707 | −.0414 | −37 | 1185 | 1193 | QRELEQL |
| | | | | | | GR |
| 1142.6528 | 1142.6127 | −.0401 | −35 | 1436 | 1444 | IEEEIRVVR |
| 1161.6296 | 1161.6194 | −.0102 | −9 | 4325 | 4334 | GLVDKIMV |
| | | | | | | DR |
| 1177.5994 | 1177.6208 | .0214 | 18 | 2534 | 2543 | LVASMEE |
| | | | | | | ARR |
| 1193.5765 | 1193.6279 | .0514 | 43 | 968 | 976 | CQRCISELK |
| 1220.6344 | 1220.6071 | −.0273 | −22 | 3979 | 3989 | MGIVGPEF |
| | | | | | | KDK |
| 1220.6555 | 1220.6071 | −.0484 | −40 | 2221 | 2230 | VQMEELS |
| | | | | | | KLK |
| 1268.6191 | 1268.6755 | .0564 | 44 | 2251 | 2260 | FLQEEAEK |
| | | | | | | MK |
| 1268.6191 | 1268.6755 | .0564 | 44 | 2251 | 2260 | FLQEEAEK |
| | | | | | | MK |
| 1429.8236 | 1429.7775 | −.0461 | −32 | 272 | 282 | ELVLLLLQ |
| | | | | | | WMR |
| 1445.8112 | 1445.7743 | −.0369 | −26 | 86 | 97 | LQNVQIALDYLR |
| 1463.858 | 1463.9216 | .0636 | 43 | 1277 | 1290 | AQLEPVASPAKK |
| | | | | | | PK |
| 1477.7316 | 1477.7634 | .0318 | 22 | 3886 | 3898 | SQVMDEATALQLR |
| 1495.7792 | 1495.6732 | −.106 | −71 | 2889 | 2902 | DVFEKATVSAPF |
| | | | | | | GK |
| 1495.7792 | 1495.6732 | −.106 | −71 | 2889 | 2902 | DVFEKATVSAPF |
| | | | | | | GK |
| 1510.702 | 1510.6783 | −.0237 | −16 | 4517 | 4533 | YASGSSASLGGP |
| | | | | | | ESAVA |
| 1512.6925 | 1512.702 | .0095 | 6 | 3818 | 3830 | DTHDQLSEPSEVR |
| 1528.739 | 1528.6807 | −.0583 | −38 | 1200 | 1213 | ESADPLGAWLQ |
| | | | | | | DAR |
| 1528.739 | 1528.6807 | −.0583 | −38 | 1200 | 1213 | ESADPLGAWLQ |
| | | | | | | DAR |
| 1544.785 | 1544.7278 | −.0572 | −37 | 1378 | 1389 | ELQQRMQEEVVR |
| 1566.7759 | 1566.7615 | −.0144 | −9 | 2874 | 2888 | AAKGGELVYTDS |
| | | | | | | EAR |
| 1613.8745 | 1613.7207 | −.1538 | −95 | 3119 | 3134 | LLDAQLSTGGIV |
| | | | | | | DPSK |
| 1667.885 | 1667.7976 | −.0874 | −52 | 1100 | 1115 | EAQAVPATLPEL |
| | | | | | | EATK |
| 1684.8402 | 1684.8882 | .048 | 28 | 1200 | 1214 | ESADPLGAWLQ |
| | | | | | | DARR |
| 1684.8402 | 1684.8882 | .048 | 28 | 1200 | 1214 | ESADPLGAWLQ |
| | | | | | | DARR |
| 1726.9083 | 1726.808 | −.1003 | −58 | 3852 | 3867 | RDDGTGQLLLPL |
| | | | | | | SDAR |
| 1869.9777 | 1869.87 | −.1077 | −58 | 2261 | 2278 | QVAEEAARLSVA |
| | | | | | | AQEAAR |
| 1883.887 | 1883.8921 | .0051 | 3 | 3535 | 3550 | AEVVETTQVYTE |
| | | | | | | EETR |
| 1915.947 | 1915.8633 | −.0837 | −44 | 3759 | 3774 | DPYTEQTISLFQA |
| | | | | | | MKK |
| 2480.3396 | 2480.2053 | −.1343 | −54 | 3633 | 3653 | LTAEDLFEARIISL |
| | | | | | | ETYNLLR |
| 2480.3396 | 2480.2053 | −.1343 | −54 | 3633 | 3653 | LTAEDLFEARIISL |
| | | | | | | ETYNLL R |

| | |
|---|---|
| plectin 7 [Homo sapiens] | gi\|40849938 |

Protein Group

| | |
|---|---|
| plectin isoform 1d [Homo sapiens] | gi\|41322910 |

Peptide Information

| | | | | Start | | |
|---|---|---|---|---|---|---|
| Calc. Mass | Obsrv. Mass | ±da | ±ppm | Seq. | End Seq. | Sequence |
| 819.4393 | 819.3938 | −.0455 | −56 | 4336 | 4343 | MSAAQALK |
| 828.3668 | 828.4194 | .0526 | 63 | 716 | 721 | KYSCDR |
| 860.4108 | 860.4073 | −.0035 | −4 | 2078 | 2084 | QVEEAER |
| 860.422 | 860.4073 | −.0147 | −17 | 1591 | 1597 | AREEAER |
| 930.489 | 930.4886 | −.0004 | 0 | 2418 | 2425 | EAIAELER |
| 956.4506 | 956.4131 | −.0375 | −39 | 624 | 631 | ESYSALMR |
| 1036.5786 | 1036.5348 | −.0438 | −42 | 454 | 461 | TEYNLRLK |
| 1037.6255 | 1037.5444 | −.0811 | −78 | 19 | 26 | WVNKHLIK |
| 1068.5143 | 1068.5271 | .0128 | 12 | 139 | 147 | MVEGYQGLR |
| 1084.5455 | 1084.5914 | .0459 | 42 | 623 | 631 | KESYSALMR |
| 1107.5729 | 1107.5511 | −.0218 | −20 | 2938 | 2946 | GRLCFEGLR |
| 1128.6121 | 1128.5707 | −.0414 | −37 | 1167 | 1175 | QRELEQLGR |
| 1142.6528 | 1142.6127 | −.0401 | −35 | 1418 | 1426 | IEEEIRVVR |
| 1161.6296 | 1161.6194 | −.0102 | −9 | 4307 | 4316 | GLVDKIMVDR |
| 1177.5994 | 1177.6208 | .0214 | 18 | 2516 | 2525 | LVASMEEARR |
| 1193.5765 | 1193.6279 | .0514 | 43 | 950 | 958 | CQRCISELK |
| 1220.6344 | 1220.6071 | −.0273 | −22 | 3961 | 3971 | MGIVGPEFKDK |
| 1220.6555 | 1220.6071 | −.0484 | −40 | 2203 | 2212 | VQMEELSKLK |
| 1268.6191 | 1268.6755 | .0564 | 44 | 2233 | 2242 | FLQEEAEKMK |
| 1268.6191 | 1268.6755 | .0564 | 44 | 2233 | 2242 | FLQEEAEKMK |
| 1429.8236 | 1429.7775 | −.0461 | −32 | 254 | 264 | ELVLLLLQWMR |
| 1445.8112 | 1445.7743 | −.0369 | −26 | 68 | 79 | LQNVQIALDYLR |
| 1463.858 | 1463.9216 | .0636 | 43 | 1259 | 1272 | AQLEPVASPAKK |
| | | | | | | PK |
| 1477.7316 | 1477.7634 | .0318 | 22 | 3868 | 3880 | SQVMDEATALQ |
| | | | | | | LR |
| 1495.7792 | 1495.6732 | −.106 | −71 | 2871 | 2884 | DVFEKATVSAPF |
| | | | | | | GK |
| 1495.7792 | 1495.6732 | −.106 | −71 | 2871 | 2884 | DVFEKATVSAPF |
| | | | | | | GK |
| 1510.702 | 1510.6783 | −.0237 | −16 | 4499 | 4515 | YASGSSASLGG |
| | | | | | | PESAVA |
| 1512.6925 | 1512.702 | .0095 | 6 | 3800 | 3812 | DTHDQLSEPSE |
| | | | | | | VR |
| 1528.739 | 1528.6807 | −.0583 | −38 | 1182 | 1195 | ESADPLGAWLQ |
| | | | | | | DAR |
| 1528.739 | 1528.6807 | −.0583 | −38 | 1182 | 1195 | ESADPLGAWLQ |
| | | | | | | DAR |
| 1544.785 | 1544.7278 | −.0572 | −37 | 1360 | 1371 | ELQQRMQEEVVR |
| 1566.7759 | 1566.7615 | −.0144 | −9 | 2856 | 2870 | AAKGGELVYTD |
| | | | | | | SEAR |
| 1613.8745 | 1613.7207 | −.1538 | −95 | 3101 | 3116 | LLDAQLSTGGIV |
| | | | | | | DPSK |
| 1667.885 | 1667.7976 | −.0874 | −52 | 1082 | 1097 | EAQAVPATLPEL |
| | | | | | | EATK |
| 1684.8402 | 1684.8882 | .048 | 28 | 1182 | 1196 | ESADPLGAWLQ |
| | | | | | | DARR |
| 1684.8402 | 1684.8882 | .048 | 28 | 1182 | 1196 | ESADPLGAWLQ |
| | | | | | | DARR |
| 1726.9083 | 1726.808 | −.1003 | −58 | 3834 | 3849 | RDDGTGQLLLPL |
| | | | | | | SDAR |
| 1869.9777 | 1869.87 | −.1077 | −58 | 2243 | 2260 | QVAEEAARLSVA |
| | | | | | | AQEAAR |
| 1883.887 | 1883.8921 | .0051 | 3 | 3517 | 3532 | AEVVETTQVYTE |
| | | | | | | EETR |
| 1915.947 | 1915.8633 | −.0837 | −44 | 3741 | 3756 | DPYTEQTISLFQA |
| | | | | | | MKK |
| 2480.3396 | 2480.2053 | −.1343 | −54 | 3615 | 3635 | LTAEDLFEARIISL |
| | | | | | | ETYNLLR |
| 2480.3396 | 2480.2053 | −.1343 | −54 | 3615 | 3635 | LTAEDLFEARIISL |
| | | | | | | ETYNLLR |

| | |
|---|---|
| plectin 10 [Homo sapiens] | gi\|40849942 |

Protein Group

| | |
|---|---|
| plectin isoform 1g [Homo sapiens] | gi\|41322914 |

Peptide Information

| | | | | Start | | |
|---|---|---|---|---|---|---|
| Calc. Mass | Obsrv. Mass | ±da | ±ppm | Seq. | End Seq. | Sequence |
| 819.4393 | 819.3938 | −.0455 | −56 | 4372 | 4379 | MSAAQALK |
| 828.3668 | 828.4194 | .0526 | 63 | 752 | 757 | KYSCDR |
| 860.4108 | 860.4073 | −.0035 | −4 | 2114 | 2120 | QVEEAER |
| 860.422 | 860.4073 | −.0147 | −17 | 1627 | 1633 | AREEAER |
| 930.489 | 930.4886 | −.0004 | 0 | 2454 | 2461 | EAIAELER |
| 956.4506 | 956.4131 | −.0375 | −39 | 660 | 667 | ESYSALMR |
| 1036.5786 | 1036.5348 | −.0438 | −42 | 490 | 497 | TEYNLRLK |
| 1037.6255 | 1037.5444 | −.0811 | −78 | 55 | 62 | WVNKHLIK |
| 1068.5143 | 1068.5271 | .0128 | 12 | 175 | 183 | MVEGYQGLR |
| 1084.5455 | 1084.5914 | .0459 | 42 | 659 | 667 | KESYSALMR |
| 1107.5729 | 1107.5511 | −.0218 | −20 | 2974 | 2982 | GRLCFEGLR |
| 1128.6121 | 1128.5707 | −.0414 | −37 | 1203 | 1211 | QRELEQLGR |
| 1142.6528 | 1142.6127 | −.0401 | −35 | 1454 | 1462 | IEEEIRVVR |
| 1161.6296 | 1161.6194 | −.0102 | −9 | 4343 | 4352 | GLVDKIMVDR |
| 1177.5994 | 1177.6208 | .0214 | 18 | 2552 | 2561 | LVASMEEARR |
| 1193.5765 | 1193.6279 | .0514 | 43 | 986 | 994 | CQRCISELK |
| 1220.6344 | 1220.6071 | −.0273 | −22 | 3997 | 4007 | MGIVGPEFKDK |
| 1220.6555 | 1220.6071 | −.0484 | −40 | 2239 | 2248 | VQMEELSKLK |
| 1268.6191 | 1268.6755 | .0564 | 44 | 2269 | 2278 | FLQEEAEKMK |
| | 1268.6755 | | | | | |
| 1268.6191 | 1268.6755 | .0564 | 44 | 2269 | 2278 | FLQEEAEKMK |
| 1429.8236 | 1429.7775 | −.0461 | −32 | 290 | 300 | ELVLLLLQWMR |
| 1445.8112 | 1445.7743 | −.0369 | −26 | 104 | 115 | LQNVQIALDYLR |
| 1463.858 | 1463.9216 | .0636 | 43 | 1295 | 1308 | AQLEPVASPAKK |
| | | | | | | PK |
| 1477.7316 | 1477.7634 | .0318 | 22 | 3904 | 3916 | SQVMDEATALQLR |
| 1495.7792 | 1495.6732 | −.106 | −71 | 2907 | 2920 | DVFEKATVSAPF |
| | | | | | | GK |
| 1495.7792 | 1495.6732 | −.106 | −71 | 2907 | 2920 | DVFEKATVSAPF |
| | | | | | | GK |
| 1510.702 | 1510.6783 | −.0237 | −16 | 4535 | 4551 | YASGSSASLGGP |
| | | | | | | ESAVA |
| 1512.6925 | 1512.702 | .0095 | 6 | 3836 | 3848 | DTHDQLSEPSEVR |
| 1528.739 | 1528.6807 | −.0583 | −38 | 1218 | 1231 | ESADPLGAWLQ |
| | | | | | | DAR |
| 1528.739 | 1528.6807 | −.0583 | −38 | 1218 | 1231 | ESADPLGAWLQ |
| | | | | | | DAR |
| 1544.785 | 1544.7278 | −.0572 | −37 | 1396 | 1407 | ELQQRMQEEVVR |
| 1566.7759 | 1566.7615 | −.0144 | −9 | 2892 | 2906 | AAKGGELVYTDS |
| | | | | | | EAR |
| 1613.8745 | 1613.7207 | −.1538 | −95 | 3137 | 3152 | LLDAQLSTGGIV |
| | | | | | | DPSK |
| 1667.885 | 1667.7976 | −.0874 | −52 | 1118 | 1133 | EAQAVPATLPEL |
| | | | | | | EATK |
| 1684.8402 | 1684.8882 | .048 | 28 | 1218 | 1232 | ESADPLGAWLQ |
| | | | | | | DARR |
| 1684.8402 | 1684.8882 | .048 | 28 | 1218 | 1232 | ESADPLGAWLQ |
| | | | | | | DARR |
| 1726.9083 | 1726.808 | −.1003 | −58 | 3870 | 3885 | RDDGTGQLLLPL |
| | | | | | | SDAR |
| 1869.9777 | 1869.87 | −.1077 | −58 | 2279 | 2296 | QVAEEAARLSVA |
| | | | | | | AQEAAR |
| 1883.887 | 1883.8921 | .0051 | 3 | 3553 | 3568 | AEVVETTQVYTE |
| | | | | | | EETR |
| 1915.947 | 1915.8633 | −.0837 | −44 | 3777 | 3792 | DPYTEQTISLFQA |
| | | | | | | MKK |
| 2480.3396 | 2480.2053 | −.1343 | −54 | 3651 | 3671 | LTAEDLFEARIISL |
| | | | | | | ETYNLLR |
| 2480.3396 | 2480.2053 | −.1343 | −54 | 3651 | 3671 | LTAEDLFEARIISL |
| | | | | | | ETYNLLR |

| | |
|---|---|
| plectin 1 [Homo sapiens] | gi\|40849930 |

Protein Group

| | |
|---|---|
| plectin isoform 1c [Homo sapiens] | gi\|47607492 |

Peptide Information

| | | | | Start | | |
|---|---|---|---|---|---|---|
| Calc. Mass | Obsrv. Mass | ±da | ±ppm | Seq. | End Seq. | Sequence |
| 819.4393 | 819.3938 | −.0455 | −56 | 4395 | 4402 | MSAAQALK |
| 828.3668 | 828.4194 | .0526 | 63 | 775 | 780 | KYSCDR |
| 860.4108 | 860.4073 | −.0035 | −4 | 2137 | 2143 | QVEEAER |
| 860.422 | 860.4073 | −.0147 | −17 | 1650 | 1656 | AREEAER |
| 930.489 | 930.4886 | −.0004 | 0 | 2477 | 2484 | EAIAELER |
| 956.4506 | 956.4131 | −.0375 | −39 | 683 | 690 | ESYSALMR |
| 1036.5786 | 1036.5348 | −.0438 | −42 | 513 | 520 | TEYNLRLK |
| 1037.6255 | 1037.5444 | −.0811 | −78 | 78 | 85 | WVNKHLIK |
| 1068.5143 | 1068.5271 | .0128 | 12 | 198 | 206 | MVEGYQGLR |
| 1084.5455 | 1084.5914 | .0459 | 42 | 682 | 690 | KESYSALMR |
| 1107.5729 | 1107.5511 | −.0218 | −20 | 2997 | 3005 | GRLCFEGLR |
| 1128.6121 | 1128.5707 | −.0414 | −37 | 1226 | 1234 | QRELEQLGR |
| 1142.6528 | 1142.6127 | −.0401 | −35 | 1477 | 1485 | IEEEIRVVR |
| 1161.6296 | 1161.6194 | −.0102 | −9 | 4366 | 4375 | GLVDKIMVDR |
| 1177.5994 | 1177.6208 | .0214 | 18 | 2575 | 2584 | LVASMEEARR |
| 1193.5765 | 1193.6279 | .0514 | 43 | 1009 | 1017 | CQRCISELK |
| 1220.6344 | 1220.6071 | −.0273 | −22 | 4020 | 4030 | MGIVGPEFKDK |
| 1220.6555 | 1220.6071 | −.0484 | −40 | 2262 | 2271 | VQMEELSKLK |
| 1268.6191 | 1268.6755 | .0564 | 44 | 2292 | 2301 | FLQEEAEKMK |
| 1268.6191 | 1268.6755 | .0564 | 44 | 2292 | 2301 | FLQEEAEKMK |
| 1429.8236 | 1429.7775 | −.0461 | −32 | 313 | 323 | ELVLLLLQWMR |
| 1445.8112 | 1445.7743 | −.0369 | −26 | 127 | 138 | LQNVQIALDYLR |
| 1463.858 | 1463.9216 | .0636 | 43 | 1318 | 1331 | AQLEPVASPAKK |
| | | | | | | PK |
| 1477.7316 | 1477.7634 | .0318 | 22 | 3927 | 3939 | SQVMDEATALQ |
| | | | | | | LR |
| 1495.7792 | 1495.6732 | −.106 | −71 | 2930 | 2943 | DVFEKATVSAPF |
| | | | | | | GK |
| 1495.7792 | 1495.6732 | −.106 | −71 | 2930 | 2943 | DVFEKATVSAPF |
| | | | | | | GK |
| 1510.702 | 1510.6783 | −.0237 | −16 | 4558 | 4574 | YASGSSASLGG |
| | | | | | | PESAVA |
| 1512.6925 | 1512.702 | .0095 | 6 | 3859 | 3871 | DTHDQLSEPSE |
| | | | | | | VR |
| 1528.739 | 1528.6807 | −.0583 | −38 | 1241 | 1254 | ESADPLGAWLQ |
| | | | | | | DAR |
| 1528.739 | 1528.6807 | −.0583 | −38 | 1241 | 1254 | ESADPLGAWLQ |
| | | | | | | DAR |
| 1544.785 | 1544.7278 | −.0572 | −37 | 1419 | 1430 | ELQQRMQEEVVR |
| 1566.7759 | 1566.7615 | −.0144 | −9 | 2915 | 2929 | AAKGGELVYTD |
| | | | | | | SEAR |
| 1613.8745 | 1613.7207 | −.1538 | −95 | 3160 | 3175 | LLDAQLSTGGIV |
| | | | | | | DPSK |
| 1667.885 | 1667.7976 | −.0874 | −52 | 1141 | 1156 | EAQAVPATLPEL |
| | | | | | | EATK |
| 1684.8402 | 1684.8882 | .048 | 28 | 1241 | 1255 | ESADPLGAWLQ |
| | | | | | | DARR |
| 1684.8402 | 1684.8882 | .048 | 28 | 1241 | 1255 | ESADPLGAWLQ |
| | | | | | | DARR |
| 1726.9083 | 1726.808 | −.1003 | −58 | 3893 | 3908 | RDDGTGQLLLPL |
| | | | | | | SDAR |
| 1869.9777 | 1869.87 | −.1077 | −58 | 2302 | 2319 | QVAEEAARLSVA |
| | | | | | | AQEAAR |
| 1883.887 | 1883.8921 | .0051 | 3 | 3576 | 3591 | AEVVETTQVYTE |
| | | | | | | EETR |
| 1915.947 | 1915.8633 | −.0837 | −44 | 3800 | 3815 | DPYTEQTISLFQA |
| | | | | | | MKK |
| 2480.3396 | 2480.2053 | −.1343 | −54 | 3674 | 3694 | LTAEDLFEARIISL |
| | | | | | | ETYNLLR |
| | 2480.2053 | | | | | |
| 2480.3396 | 2480.2053 | −.1343 | −54 | 3674 | 3694 | LTAEDLFEARIISL |
| | | | | | | ETYNLLR |

| | |
|---|---|
| plectin [Homo sapiens] | gi\|1477651 |

Protein Group

| | |
|---|---|
| plectin [Homo sapiens] | gi\|1477646 |

Peptide Information

| | | | | Start | | |
|---|---|---|---|---|---|---|
| Calc. Mass | Obsrv. Mass | ±da | ±ppm | Seq. | End Seq. | Sequence |
| 819.4393 | 819.3938 | −.0455 | −56 | 4395 | 4402 | MSAAQALK |
| 828.3668 | 828.4194 | .0526 | 63 | 775 | 780 | KYSCDR |
| 860.4108 | 860.4073 | −.0035 | −4 | 2137 | 2143 | QVEEAER |
| 860.422 | 860.4073 | −.0147 | −17 | 1650 | 1656 | AREEAER |
| 930.489 | 930.4886 | −.0004 | 0 | 2477 | 2484 | EAIAELER |
| 956.4506 | 956.4131 | −.0375 | −39 | 683 | 690 | ESYSALMR |
| 1036.5786 | 1036.5348 | −.0438 | −42 | 513 | 520 | TEYNLRLK |
| 1037.6255 | 1037.5444 | −.0811 | −78 | 78 | 85 | WVNKHLIK |
| 1068.5143 | 1068.5271 | .0128 | 12 | 198 | 206 | MVEGYQGLR |
| 1084.5455 | 1084.5914 | .0459 | 42 | 682 | 690 | KESYSALMR |
| 1107.5729 | 1107.5511 | −.0218 | −20 | 2997 | 3005 | GRLCFEGLR |
| 1128.6121 | 1128.5707 | −.0414 | −37 | 1226 | 1234 | QRELEQLGR |
| 1142.6528 | 1142.6127 | −.0401 | −35 | 1477 | 1485 | IEEEIRVVR |
| 1161.6296 | 1161.6194 | −.0102 | −9 | 4366 | 4375 | GLVDKIMVDR |
| 1177.5994 | 1177.6208 | .0214 | 18 | 2575 | 2584 | LVASMEEARR |
| 1193.5765 | 1193.6279 | .0514 | 43 | 1009 | 1017 | CQRCISELK |
| 1220.6344 | 1220.6071 | −.0273 | −22 | 4020 | 4030 | MGIVGPEFKDK |
| 1220.6555 | 1220.6071 | −.0484 | −40 | 2262 | 2271 | VQMEELSKLK |
| 1268.6191 | 1268.6755 | .0564 | 44 | 2292 | 2301 | FLQEEAEKMK |
| 1268.6191 | 1268.6755 | .0564 | 44 | 2292 | 2301 | FLQEEAEKMK |
| 1429.8236 | 1429.7775 | −.0461 | −32 | 313 | 323 | ELVLLLLQWMR |
| 1445.8112 | 1445.7743 | −.0369 | −26 | 127 | 138 | LQNVQIALDYLR |
| 1463.858 | 1463.9216 | .0636 | 43 | 1318 | 1331 | AQLEPVASPAKK |
| | | | | | | PK |
| 1477.7316 | 1477.7634 | .0318 | 22 | 3927 | 3939 | SQVMDEATALQLR |
| 1495.7792 | 1495.6732 | −.106 | −71 | 2930 | 2943 | DVFEKATVSA |
| | | | | | | PFGK |
| 1495.7792 | 1495.6732 | −.106 | −71 | 2930 | 2943 | DVFEKATVSA |
| | | | | | | PFGK |
| 1510.702 | 1510.6783 | −.0237 | −16 | 4558 | 4574 | YASGSSASLGGP |
| | | | | | | ESAVA |
| 1512.6925 | 1512.702 | .0095 | 6 | 3859 | 3871 | DTHDQLSEPSEVR |
| 1528.739 | 1528.6807 | −.0583 | −38 | 1241 | 1254 | ESADPLGAWLQ |
| | | | | | | DAR |
| 1528.739 | 1528.6807 | −.0583 | −38 | 1241 | 1254 | ESADPLGAWLQ |
| | | | | | | DAR |
| 1544.785 | 1544.7278 | −.0572 | −37 | 1419 | 1430 | ELQQRMQEEVVR |
| 1566.8347 | 1566.7615 | −.0732 | −47 | 2699 | 2712 | LAQGHTTVDELA |
| | | | | | | RR |
| 1613.8745 | 1613.7207 | −.1538 | −95 | 3160 | 3175 | LLDAQLSTGGIV |
| | | | | | | DPSK |
| 1667.885 | 1667.7976 | −.0874 | −52 | 1141 | 1156 | EAQAVPATLPEL |
| | | | | | | EATK |
| 1684.8402 | 1684.8882 | .048 | 28 | 1241 | 1255 | ESADPLGAWLQ |
| | | | | | | DARR |
| 1684.8402 | 1684.8882 | .048 | 28 | 1241 | 1255 | ESADPLGAWLQ |
| | | | | | | DARR |
| 1726.9083 | 1726.808 | −.1003 | −58 | 3893 | 3908 | RDDGTGQLLLPL |
| | | | | | | SDAR |
| 1869.9777 | 1869.87 | −.1077 | −58 | 2302 | 2319 | QVAEEAARLSVA |
| | | | | | | AQEAAR |
| 1883.887 | 1883.8921 | .0051 | 3 | 3576 | 3591 | AEVVETTQVYTE |
| | | | | | | EETR |
| 1915.947 | 1915.8633 | −.0837 | −44 | 3800 | 3815 | DPYTEQTISLFQA |
| | | | | | | MKK |
| 2480.3396 | 2480.2053 | −.1343 | −54 | 3674 | 3694 | LTAEDLFEARIISL |
| | | | | | | ETYNLLR |
| 2480.3396 | 2480.2053 | −.1343 | −54 | 3674 | 3694 | LTAEDLFEARIISL |
| | | | | | | ETYNLLR |

| | |
|---|---|
| plectin isoform 1 [Homo sapiens] | gi\|41322916 |

Protein Group

| | |
|---|---|
| RecName: Full = Plectin; Short = PCN; Short = PLTN; | gi\|209572726 |
| AltName: Full = Hemidesmosomal | |
| protein 1; Short = HD1; | |
| AltName: Full = Plectin-1 | |
| plectin 6 [Homo sapiens] | gi\|40849936 |
| unnamed protein product [Homo sapiens] | gi\|218103024 |
| unnamed protein product [Homo sapiens] | gi\|211637717 |
| unnamed protein product [Homo sapiens] | gi\|158066003 |
| unnamed protein product [Homo sapiens] | gi\|157391371 |

Peptide Information

| | | | | Start | | |
|---|---|---|---|---|---|---|
| Calc. Mass | Obsrv. Mass | ±da | ±ppm | Seq. | End Seq. | Sequence |
| 819.4393 | 819.3938 | −.0455 | −56 | 4505 | 4512 | MSAAQALK |
| 828.3668 | 828.4194 | .0526 | 63 | 885 | 890 | KYSCDR |
| 860.4108 | 860.4073 | −.0035 | −4 | 2247 | 2253 | QVEEAER |
| 860.422 | 860.4073 | −.0147 | −17 | 1760 | 1766 | AREEAER |
| 930.489 | 930.4886 | −.0004 | 0 | 2587 | 2594 | EAIAELER |
| 956.4506 | 956.4131 | −.0375 | −39 | 793 | 800 | ESYSALMR |
| 1036.5786 | 1036.5348 | −.0438 | −42 | 623 | 630 | TEYNLRLK |
| 1037.4941 | 1037.5444 | .0503 | 48 | 1 | 9 | MVAGMLMPR |
| 1068.5143 | 1068.5271 | .0128 | 12 | 308 | 316 | MVEGYQGLR |
| 1084.5455 | 1084.5914 | .0459 | 42 | 792 | 800 | KESYSALMR |
| 1107.5729 | 1107.5511 | −.0218 | −20 | 3107 | 3115 | GRLCFEGLR |
| 1128.6121 | 1128.5707 | −.0414 | −37 | 1336 | 1344 | QRELEQLGR |
| 1142.6528 | 1142.6127 | −.0401 | −35 | 1587 | 1595 | IEEEIRVVR |
| 1161.6296 | 1161.6194 | −.0102 | −9 | 4476 | 4485 | GLVDKIMVDR |
| 1177.5994 | 1177.6208 | .0214 | 18 | 2685 | 2694 | LVASMEEARR |
| 1193.5765 | 1193.6279 | .0514 | 43 | 1119 | 1127 | CQRCISELK |
| 1220.6344 | 1220.6071 | −.0273 | −22 | 4130 | 4140 | MGIVGPEFKDK |
| 1220.6555 | 1220.6071 | −.0484 | −40 | 2372 | 2381 | VQMEELSKLK |
| 1268.6191 | 1268.6755 | .0564 | 44 | 2402 | 2411 | FLQEEAEKMK |
| 1268.6191 | 1268.6755 | .0564 | 44 | 2402 | 2411 | FLQEEAEKMK |
| 1429.8236 | 1429.7775 | −.0461 | −32 | 423 | 433 | ELVLLLLQWMR |
| 1445.8112 | 1445.7743 | −.0369 | −26 | 237 | 248 | LQNVQIALDYLR |
| 1463.858 | 1463.9216 | .0636 | 43 | 1428 | 1441 | AQLEPVASPAKK |
| | | | | | | PK |
| 1477.7316 | 1477.7634 | .0318 | 22 | 4037 | 4049 | SQVMDEATALQLR |
| 1495.7792 | 1495.6732 | −.106 | −71 | 3040 | 3053 | DVFEKATVSAPF |
| | | | | | | GK |
| 1495.7792 | 1495.6732 | −.106 | −71 | 3040 | 3053 | DVFEKATVSAPF |
| | | | | | | GK |
| 1510.702 | 1510.6783 | −.0237 | −16 | 4668 | 4684 | YASGSSASLGGP |
| | | | | | | ESAVA |
| 1512.6925 | 1512.702 | .0095 | 6 | 3969 | 3981 | DTHDQLSEPSEVR |
| 1528.739 | 1528.6807 | −.0583 | −38 | 1351 | 1364 | ESADPLGAWLQD |
| | | | | | | AR |
| 1528.739 | 1528.6807 | −.0583 | −38 | 1351 | 1364 | ESADPLGAWLQD |
| | | | | | | AR |
| 1544.785 | 1544.7278 | −.0572 | −37 | 1529 | 1540 | ELQQRMQEEVVR |
| 1566.7759 | 1566.7615 | −.0144 | −9 | 3025 | 3039 | AAKGGELVYTDS |
| | | | | | | EAR |
| 1613.8745 | 1613.7207 | −.1538 | −95 | 3270 | 3285 | LLDAQLSTGGIVD |
| | | | | | | PSK |
| 1667.885 | 1667.7976 | −.0874 | −52 | 1251 | 1266 | EAQAVPATLPELE |
| | | | | | | ATK |
| 1684.8402 | 1684.8882 | .048 | 28 | 1351 | 1365 | ESADPLGAWLQD |
| | | | | | | ARR |
| 1684.8402 | 1684.8882 | .048 | 28 | 1351 | 1365 | ESADPLGAWLQ |
| | | | | | | DARR |
| 1726.9083 | 1726.808 | −.1003 | −58 | 4003 | 4018 | RDDGTGQLLLPL |
| | | | | | | SDAR |
| 1869.9777 | 1869.87 | −.1077 | −58 | 2412 | 2429 | QVAEEAARLSV |
| | | | | | | AAQEAAR |
| 1883.887 | 1883.8921 | .0051 | 3 | 3686 | 3701 | AEVVETTQVYTE |
| | | | | | | EETR |
| 1915.947 | 1915.8633 | −.0837 | −44 | 3910 | 3925 | DPYTEQTISLFQA |
| | | | | | | MKK |
| 2480.3396 | 2480.2053 | −.1343 | −54 | 3784 | 3804 | LTAEDLFEARIISL |
| | | | | | | ETYNLLR |
| 2480.3396 | 2480.2053 | −.1343 | −54 | 3784 | 3804 | LTAEDLFEARIISL |
| | | | | | | ETYNLLR |

| | |
|---|---|
| hypothetical protein LOC134121 [Homo sapiens] | gi\|148237105 |

Protein Group

| | |
|---|---|
| Chromosome 5 open reading frame 49 [Homo sapiens] | gi\|144719176 |
| Chromosome 5 open reading frame 49 [Homo sapiens] | gi\|144719146 |
| RecName: Full = Uncharacterized protein C5orf49 | gi\|189041165 |
| hCG1643563 [Homo sapiens] | gi\|119628501 |

Peptide Information

| | | | | Start | | |
|---|---|---|---|---|---|---|
| Calc. Mass | Obsrv. Mass | ±da | ±ppm | Seq. | End Seq. | Sequence |
| 811.4169 | 811.4042 | −.0127 | −16 | 70 | 75 | LHRDDR |
| 870.4064 | 870.48 | .0736 | 85 | 73 | 79 | DDREHAK |
| 1193.6637 | 1193.6279 | −.0358 | −30 | 106 | 115 | INQPIEPLNR |
| 1220.5807 | 1220.6071 | .0264 | 22 | 120 | 129 | ANHVQADFYR |
| 1220.5807 | 1220.6071 | .0264 | 22 | 120 | 129 | ANHVQADFYR |
| 1349.7648 | 1349.6904 | −.0744 | −55 | 105 | 115 | RINQPIEPLNR |
| 1429.7396 | 1429.7775 | .0379 | 27 | 50 | 61 | TGIISLYDCIFK |
| 1495.6395 | 1495.6732 | .0337 | 23 | 2 | 14 | EDDEEETTASTLR |
| 1495.6395 | 1495.6732 | .0337 | 23 | 2 | 14 | EDDEEETTASTLR |

| | |
|---|---|
| coagulation factor V [Homo sapiens] | gi\|85718563 |

Peptide Information

| | | | | Start | | |
|---|---|---|---|---|---|---|
| Calc. Mass | Obsrv. Mass | ±da | ±ppm | Seq. | End Seq. | Sequence |
| 930.5367 | 930.4886 | −.0481 | −52 | 52 | 59 | AQVRDTLK |
| 956.4618 | 956.4131 | −.0487 | −51 | 34 | 41 | HTVNPNMK |
| 1082.6205 | 1082.5989 | −.0216 | −20 | 42 | 51 | EDGILGPIIR |
| 1487.7238 | 1487.7268 | .003 | 2 | 4 | 16 | SQHLDNFSNQI |
| | | | | | | GK |
| 1544.734 | 1544.7278 | −.0062 | −4 | 60 | 72 | VSNTNTESWE |
| | | | | | | KLH |
| 1915.9409 | 1915.8633 | −.0776 | −41 | 4 | 19 | SQHLDNFSNQI |
| | | | | | | GKHYK |

| | |
|---|---|
| PREDICTED: putative zinc finger protein | gi\|310133350 |
| 705E-like [Homo sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 811.4785 | 811.4042 | −.0743 | −92 | 108 | 114 | QIHTKGK |
| 995.5156 | 995.5454 | .0298 | 30 | 236 | 244 | AFSLSSDLR |
| 1158.452 | 1158.4729 | .0209 | 18 | 199 | 207 | WYECDNSGK |
| 1158.452 | 1158.4729 | .0209 | 18 | 199 | 207 | WYECDNSGK |
| 1193.5579 | 1193.6279 | .07 | 59 | 1 | 12 | MAAGLSANESSR |
| 1463.7974 | 1463.9216 | .1242 | 85 | 31 | 42 | THMISMHPIIRK |
| 1487.8192 | 1487.7268 | −.0924 | −62 | 13 | 24 | FPMRPPFLVIDR |
| 1495.7872 | 1495.6732 | −.114 | −76 | 31 | 42 | THMISMHPIIRK |
| 1495.7872 | 1495.6732 | −.114 | −76 | 30 | 41 | KTHMISMHPIIR |
| 2364.0317 | 2364.1492 | .1175 | 50 | 115 | 132 | SYQCNLCEKAYT |
| | | | | | | NCFHLR |
| 2364.0317 | 2364.1492 | .1175 | 50 | 115 | 132 | SYQCNLCEKAYT |
| | | | | | | NCFHLR |

| | | | |
|---|---|---|---|
| | 41/B14 | | Instr./Gel |
| | | | Origin |
| | [1] Sample Project | | Instrument |
| | 20110503 | | Sample |
| | | | Name |
| Protein Name - | KH8 (SEQ ID NO. KH8) | Accession No. | |

| | |
|---|---|
| unnamed protein product [Homo sapiens] | gi\|1335098 |

Peptide Information

| | Obsrv. | | | Start | | |
|---|---|---|---|---|---|---|
| Calc. Mass | Mass | ±da | ±ppm | Seq. | End Seq. | Sequence |
| 813.4399 | 813.4328 | −.0071 | −9 | 349 | 355 | LHIMAGR |
| 828.4111 | 828.4098 | −.0013 | −2 | 55 | 60 | SHKWDR |
| 1070.5742 | 1070.5739 | −.0003 | 0 | 191 | 199 | GEVPPRYPR |
| 1129.6517 | 1129.6481 | −.0036 | −3 | 356 | 363 | RLWWLDLK |
| 1141.5889 | 1141.5925 | .0036 | 3 | 310 | 320 | GGYTLVSG |
| | | | | | | YPK |
| 1142.6317 | 1142.6134 | −.0183 | −16 | 80 | 89 | QGHNSVFLIK |
| 1158.4707 | 1158.472 | .0013 | 1 | 203 | 211 | DYFMPCPGR |
| 1158.4707 | 1158.472 | .0013 | 1 | 203 | 211 | DYFMPCPGR |
| 1220.606 | 1220.6074 | .0014 | 1 | 69 | 79 | NFPSPVDA |
| | | | | | | AFR |
| 1220.606 | 1220.6074 | .0014 | 1 | 69 | 79 | NFPSPVDA |
| | | | | | | AFR |
| 1268.6747 | 1268.674 | −.0007 | −1 | 186 | 196 | FDPVRGEV |
| | | | | | | PPR |
| 1297.6899 | 1297.6924 | .0025 | 2 | 310 | 321 | GGYTLVSG |
| | | | | | | YPKR |
| 1495.6787 | 1495.6833 | .0046 | 3 | 175 | 185 | YYCFQGNQFLR |
| 1495.6787 | 1495.6833 | .0046 | 3 | 175 | 185 | YYCFQGNQFLR |
| 1512.6722 | 1512.6998 | .0276 | 18 | 200 | 211 | DVRDYFMPCPGR |
| 1528.6672 | 1528.6842 | .017 | 11 | 200 | 211 | DVRDYFMPCPGR |
| 1528.6672 | 1528.6842 | .017 | 11 | 200 | 211 | DVRDYFMPCPGR |
| 1534.7802 | 1534.7842 | .004 | 3 | 67 | 79 | WKNFPSPVDAA |
| | | | | | | FR |
| 1712.7698 | 1712.8146 | .0448 | 26 | 129 | 143 | GECQAEGVLFF |
| | | | | | | QGDR |
| 1837.8868 | 1837.9355 | .0487 | 26 | 364 | 379 | SGAQATWTELP |
| | | | | | | WPHEK |
| 2364.1653 | 2364.1904 | .0251 | 11 | 108 | 128 | LLQDEFPGIPSP |
| | | | | | | LDAAVE |
| | | | | | | CHR |
| 2364.1653 | 2364.1904 | .0251 | 11 | 108 | 128 | LLQDEFPGIPSP |
| | | | | | | LDAAVE |
| | | | | | | CHR |
| 2498.2344 | 2498.2678 | .0334 | 13 | 325 | 348 | EVGTPHGIILDSV |
| | | | | | | DAAFIC |
| | | | | | | PGSSR |
| 2498.2344 | 2498.2678 | .0334 | 13 | 325 | 348 | EVGTPHGIILDSV |
| | | | | | | DAAFIC PGSSR |

| | |
|---|---|
| hemopexin, isoform CRA_b [Homo sapiens] | gi\|119589125 |

Protein Group

| | |
|---|---|
| RecName: Full = Hemopexin; AltName: | gi\|1708182 |
| Full = Beta-1B- | |
| glycoprotein; Flags: | |
| Precursor | |
| hemopexin [Homo sapiens] | gi\|184497 |
| hemopexin precursor [Homo sapiens] | gi\|11321561 |
| hemopexin precursor [Homo sapiens] | gi\|386789 |
| unnamed protein product [Homo sapiens] | gi\|189053897 |
| unnamed protein product [Homo sapiens] | |
| unnamed protein product [Homo sapiens] | gi\|80747621 |

Peptide Information

| | Obsrv. | | | Start | | |
|---|---|---|---|---|---|---|
| Calc. Mass | Mass | ±da | ±ppm | Seq. | End Seq. | Sequence |
| 813.4399 | 813.4328 | −.0071 | −9 | 372 | 378 | LHIMAGR |
| 828.4111 | 828.4098 | −.0013 | −2 | 78 | 83 | SHKWDR |
| 1070.5742 | 1070.5739 | −.0003 | 0 | 214 | 222 | GEVPPRYPR |
| 1129.6517 | 1129.6481 | −.0036 | −3 | 379 | 386 | RLWWLDLK |
| 1141.5889 | 1141.5925 | .0036 | 3 | 333 | 343 | GGYTLVSGYPK |
| 1142.6317 | 1142.6134 | −.0183 | −16 | 103 | 112 | QGHNSVFLIK |
| 1158.4707 | 1158.472 | .0013 | 1 | 226 | 234 | DYFMPCPGR |
| 1158.4707 | 1158.472 | .0013 | 1 | 226 | 234 | DYFMPCPGR |
| 1220.606 | 1220.6074 | .0014 | 1 | 92 | 102 | NFPSPVDAAFR |
| 1220.606 | 1220.6074 | .0014 | 1 | 92 | 102 | NFPSPVDAAFR |
| 1268.6747 | 1268.674 | −.0007 | −1 | 209 | 219 | FDPVRGEVPPR |
| 1297.6899 | 1297.6924 | .0025 | 2 | 333 | 344 | GGYTLVSGYPKR |
| 1495.6787 | 1495.6833 | .0046 | 3 | 198 | 208 | YYCFQGNQFLR |
| 1495.6787 | 1495.6833 | .0046 | 3 | 198 | 208 | YYCFQGNQFLR |
| 1512.6722 | 1512.6998 | .0276 | 18 | 223 | 234 | DVRDYFMPCPGR |
| 1528.6672 | 1528.6842 | .017 | 11 | 223 | 234 | DVRDYFMPCPGR |
| 1528.6672 | 1528.6842 | .017 | 11 | 223 | 234 | DVRDYFMPCPGR |
| 1534.7802 | 1534.7842 | .004 | 3 | 90 | 102 | WKNFPSPVDAA |
| | | | | | | FR |
| 1712.7698 | 1712.8146 | .0448 | 26 | 152 | 166 | GECQAEGVLFF |
| | | | | | | QGDR |
| 1837.8868 | 1837.9355 | .0487 | 26 | 387 | 402 | SGAQATWTELP |
| | | | | | | WPHEK |
| 2364.1653 | 2364.1904 | .0251 | 11 | 131 | 151 | LLQDEFPGIPSP |
| | | | | | | LDAAVE |
| | | | | | | CHR |
| 2364.1653 | 2364.1904 | .0251 | 11 | 131 | 151 | LLQDEFPGIPSP |
| | | | | | | LDAAVE |
| | | | | | | CHR |
| 2498.2344 | 2498.2678 | .0334 | 13 | 348 | 371 | EVGTPHGIILDSV |
| | | | | | | DAAFIC |
| | | | | | | PGSSR |
| 2498.2344 | 2498.2678 | .0334 | 13 | 348 | 371 | EVGTPHGIILDSV |
| | | | | | | DAAFIC |
| | | | | | | PGSSR |

| | |
|---|---|
| HPX protein [Homo sapiens] | gi\|13529281 |

Peptide Information

| | Obsrv. | | | Start | | |
|---|---|---|---|---|---|---|
| Calc. Mass | Mass | ±da | ±ppm | Seq. | End Seq. | Sequence |
| 828.4111 | 828.4098 | −.0013 | −2 | 78 | 83 | SHKWDR |
| 1070.5742 | 1070.5739 | −.0003 | 0 | 214 | 222 | GEVPPRYPR |
| 1142.6317 | 1142.6134 | −.0183 | −16 | 103 | 112 | QGHNSVFLIK |
| 1158.4707 | 1158.472 | .0013 | 1 | 226 | 234 | DYFMPCPGR |
| 1158.4707 | 1158.472 | .0013 | 1 | 226 | 234 | DYFMPCPGR |
| 1220.606 | 1220.6074 | .0014 | 1 | 92 | 102 | NFPSPVDAAFR |
| 1220.606 | 1220.6074 | .0014 | 1 | 92 | 102 | NFPSPVDAAFR |
| 1268.6747 | 1268.674 | −.0007 | −1 | 209 | 219 | FDPVRGEVPPR |
| 1495.6787 | 1495.6833 | .0046 | 3 | 198 | 208 | YYCFQGNQFLR |
| 1495.6787 | 1495.6833 | .0046 | 3 | 198 | 208 | YYCFQGNQFLR |
| 1512.6722 | 1512.6998 | .0276 | 18 | 223 | 234 | DVRDYFMPCPGR |
| 1528.6672 | 1528.6842 | .017 | 11 | 223 | 234 | DVRDYFMPCPGR |
| 1528.6672 | 1528.6842 | .017 | 11 | 223 | 234 | DVRDYFMPCPGR |
| 1534.7802 | 1534.7842 | .004 | 3 | 90 | 102 | WKNFPSPVDAA |
| | | | | | | FR |
| 1712.7698 | 1712.8146 | .0448 | 26 | 152 | 166 | GECQAEGVLFF |
| | | | | | | QGDR |
| 2364.1653 | 2364.1904 | .0251 | 11 | 131 | 151 | LLQDEFPGIPSP |
| | | | | | | LDAAVE |
| | | | | | | CHR |
| 2364.1653 | 2364.1904 | .0251 | 11 | 131 | 151 | LLQDEFPGIPSP |
| | | | | | | LDAAVE |
| | | | | | | CHR |

| | |
|---|---|
| hemopexin, isoform CRA_d [Homo sapiens] | gi\|119589127 |

Peptide Information

| | Obsrv. | | | Start | | |
|---|---|---|---|---|---|---|
| Calc. Mass | Mass | ±da | ±ppm | Seq. | End Seq. | Sequence |
| 813.4399 | 813.4328 | −.0071 | −9 | 301 | 307 | LHIMAGR |
| 828.4111 | 828.4098 | −.0013 | −2 | 78 | 83 | SHKWDR |
| 1129.6517 | 1129.6481 | −.0036 | −3 | 308 | 315 | RLWWLDLK |
| 1141.5889 | 1141.5925 | .0036 | 3 | 262 | 272 | GGYTLVSGYPK |
| 1142.6317 | 1142.6134 | −.0183 | −16 | 103 | 112 | QGHNSVFLIK |
| 1220.606 | 1220.6074 | .0014 | 1 | 92 | 102 | NFPSPVDAAFR |
| 1220.606 | 1220.6074 | .0014 | 1 | 92 | 102 | NFPSPVDAAFR |
| 1297.6899 | 1297.6924 | .0025 | 2 | 262 | 273 | GGYTLVSGYPKR |
| 1534.7802 | 1534.7842 | .004 | 3 | 90 | 102 | WKNFPSPVDAAFR |
| 1837.8868 | 1837.9355 | .0487 | 26 | 316 | 331 | SGAQATWTELPW |
| | | | | | | PHEK |
| 2364.1653 | 2364.1904 | .0251 | 11 | 131 | 151 | LLQDEFPGIPSPL |
| | | | | | | DAAVE |
| | | | | | | CHR |
| 2364.1653 | 2364.1904 | .0251 | 11 | 131 | 151 | LLQDEFPGIPSPL |
| | | | | | | DAAVE |
| | | | | | | CHR |
| 2498.2344 | 2498.2678 | .0334 | 13 | 277 | 300 | EVGTPHGIILDSV |
| | | | | | | DAAFIC |
| | | | | | | PGSSR |
| 2498.2344 | 2498.2678 | .0334 | 13 | 277 | 300 | EVGTPHGIILDSV |
| | | | | | | DAAFIC |
| | | | | | | PGSSR |

| | |
|---|---|
| hemopexin, isoform CRA_c [Homo sapiens] | gi\|119589126 |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4328 | −.0071 | −9 | 170 | 176 | LHIMAGR |
| 1129.6517 | 1129.6481 | −.0036 | −3 | 177 | 184 | RLWWLDLK |
| 1141.5889 | 1141.5925 | .0036 | 3 | 131 | 141 | GGYTLVSGYPK |
| 1297.6899 | 1297.6924 | .0025 | 2 | 131 | 142 | GGYTLVSGYPKR |
| 1509.6613 | 1509.719 | .0577 | 38 | 25 | 37 | SQQCLSDQGHG |
| | | | | | | HR |
| 1837.8868 | 1837.9355 | .0487 | 26 | 185 | 200 | SGAQATWTELPW |
| | | | | | | PHEK |
| 2498.2344 | 2498.2678 | .0334 | 13 | 146 | 169 | EVGTPHGIILDSV |
| | | | | | | DAAFIC |
| | | | | | | PGSSR |
| 2498.2344 | 2498.2678 | .0334 | 13 | 146 | 169 | EVGTPHGIILDSVDAAFIC |
| | | | | | | PGSSR |

| | |
|---|---|
| hemopexin, isoform CRA_a [Homo sapiens] | gi\|119589124 |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4328 | −.0071 | −9 | 118 | 124 | LHIMAGR |
| 1129.6517 | 1129.6481 | −.0036 | −3 | 125 | 132 | RLWWLDLK |
| 1141.5889 | 1141.5925 | .0036 | 3 | 79 | 89 | GGYTLVSGYPK |
| 1297.6899 | 1297.6924 | .0025 | 2 | 79 | 90 | GGYTLVSGYPKR |
| 1837.8868 | 1837.9355 | .0487 | 26 | 133 | 148 | SGAQATWTELPW |
| | | | | | | PHEK |
| 2498.2344 | 2498.2678 | .0334 | 13 | 94 | 117 | EVGTPHGIILDSV |
| | | | | | | DAAFIC |
| | | | | | | PGSSR |
| 2498.2344 | 2498.2678 | .0334 | 13 | 94 | 117 | EVGTPHGIILDSV |
| | | | | | | DAAFIC |
| | | | | | | PGSSR |

| | |
|---|---|
| hemopexin | gi\|226337 |

Peptide Information

| Calc. | Obsrv. | | | Start | End | |
|---|---|---|---|---|---|---|
| Mass | Mass | ±da | ±ppm | Seq. | Seq. | Sequence |
| 1141.5889 | 1141.5925 | .0036 | 3 | 78 | 88 | GGYTLVSG |
| | | | | | | YPK |
| 1297.6899 | 1297.6924 | .0025 | 2 | 78 | 89 | GGYTLVSG |
| | | | | | | YPKR |
| 2498.2344 | 2498.2678 | .0334 | 13 | 93 | 116 | EVGTPH |
| | | | | | | GIILDSVD |
| | | | | | | AAFICPG |
| | | | | | | SSR |
| 2498.2344 | 2498.2678 | .0334 | 13 | 93 | 116 | EVGTPH |
| | | | | | | GIILDSVD |
| | | | | | | AAFICPG |
| | | | | | | SSR |

| | |
|---|---|
| unnamed protein product | gi\|221044726 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 828.4111 | 828.4098 | −.0013 | −2 | 78 | 83 | SHKWDR |
| 1142.6317 | 1142.6134 | −.0183 | −16 | 103 | 112 | QGHNSV |
| | | | | | | FLIK |
| 1220.606 | 1220.6074 | .0014 | 1 | 92 | 102 | NFPSPVD |
| | | | | | | AAFR |
| 1220.606 | 1220.6074 | .0014 | 1 | 92 | 102 | NFPSPVD |
| | | | | | | AAFR |

| | |
|---|---|
| cingulin, | gi\|119573820 |
| isoform CRA_b [Homo | |
| sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 813.4689 | 813.4328 | −.0361 | −44 | 41 | 47 | RPAKDAR |
| 860.4724 | 860.3981 | −.0743 | −86 | 375 | 381 | LDEEVKK |
| 870.4502 | 870.476 | .0258 | 30 | 203 | 209 | MLPPEQR |
| 1129.6324 | 1129.6481 | .0157 | 14 | 357 | 367 | AVAGQGELTRK |
| 1142.6277 | 1142.6134 | −.0143 | −13 | 661 | 670 | LEEAQRGLAR |
| 1158.5209 | 1158.472 | −.0489 | −42 | 2 | 11 | EQAPNMAEPR |
| 1158.5209 | 1158.472 | −.0489 | −42 | 2 | 11 | EQAPNMAEPR |
| 1220.6052 | 1220.6074 | .0022 | 2 | 863 | 872 | TELMQERSAR |
| 1220.6052 | 1220.6074 | .0022 | 2 | 863 | 872 | TELMQERSAR |
| 1373.7384 | 1373.7284 | −.01 | −7 | 548 | 560 | ASAGDTRQVEV |
| | | | | | | LK |
| 1374.7046 | 1374.7214 | .0168 | 12 | 564 | 576 | MVAEAEATVLG |
| | | | | | | QR |
| 1544.7776 | 1544.7524 | −.0252 | −16 | 779 | 792 | QALQASQAERD |
| | | | | | | TAR |
| 1546.8007 | 1546.7771 | −.0236 | −15 | 564 | 577 | MVAEAEATVLG |
| | | | | | | QRR |
| 1837.8676 | 1837.9355 | .0679 | 37 | 993 | 1007 | EVEEQHEVNEQ |
| | | | | | | LQAR |
| 2364.1184 | 2364.1904 | .072 | 30 | 1 | 21 | MEQAPNMAEPR |
| | | | | | | GPVDH |
| | | | | | | GVQIR |
| 2364.1184 | 2364.1904 | .072 | 30 | 1 | 21 | MEQAPNMAEPR |
| | | | | | | GPVDH |
| | | | | | | GVQIR |
| 2392.1562 | 2392.2158 | .0596 | 25 | 294 | 315 | STPDLLRDQQE |
| | | | | | | AAPPGS |
| | | | | | | VDHMK |
| 2497.2505 | 2497.2788 | .0283 | 11 | 301 | 323 | DQQEAAPPGSV |
| | | | | | | DHMKAT |
| | | | | | | IYGILR |

| | |
|---|---|
| cingulin [Homo sapiens] | gi\|16262452 |

Protein Group

| | |
|---|---|
| Cingulin [Homo sapiens] | gi\|223462553 |
| Cingulin [Homo sapiens] | gi\|156230640 |
| cingulin [Homo sapiens] | gi\|8308176 |
| cingulin [Homo sapiens] | gi\|55959696 |
| cingulin, isoform CRA_a [Homo sapiens] | gi\|119573819 |
| unnamed protein product [Homo sapiens] | gi\|158261037 |

Peptide Information

| | Obsrv. | | | Start | | |
|---|---|---|---|---|---|---|
| Calc. Mass | Mass | ±da | ±ppm | Seq. | End Seq. | Sequence |
| 813.4689 | 813.4328 | −.0361 | −44 | 41 | 47 | RPAKDAR |
| 860.4724 | 860.3981 | −.0743 | −86 | 375 | 381 | LDEEVKK |
| 870.4502 | 870.476 | .0258 | 30 | 203 | 209 | MLPPEQR |
| 1129.6324 | 1129.6481 | .0157 | 14 | 357 | 367 | AVAGQGELTRK |
| 1142.6277 | 1142.6134 | −.0143 | −13 | 802 | 811 | LEEAQRGLAR |
| 1158.5209 | 1158.472 | −.0489 | −42 | 2 | 11 | EQAPNMAEPR |
| 1158.5209 | 1158.472 | −.0489 | −42 | 2 | 11 | EQAPNMAEPR |
| 1220.6052 | 1220.6074 | .0022 | 2 | 1004 | 1013 | TELMQERSAR |
| 1220.6052 | 1220.6074 | .0022 | 2 | 1004 | 1013 | TELMQERSAR |
| 1373.7384 | 1373.7284 | −.01 | −7 | 619 | 631 | ASAGDTRQVEVLK |
| 1374.7046 | 1374.7214 | .0168 | 12 | 705 | 717 | MVAEAEATVLGQR |
| 1544.7776 | 1544.7524 | −.0252 | −16 | 920 | 933 | QALQASQAERDT |
| | | | | | | AR |
| 1546.8007 | 1546.7771 | −.0236 | −15 | 705 | 718 | MVAEAEATVLGQ |
| | | | | | | RR |
| 1837.8676 | 1837.9355 | .0679 | 37 | 1134 | 1148 | EVEEQHEVNEQL |
| | | | | | | QAR |
| 2364.1184 | 2364.1904 | .072 | 30 | 1 | 21 | MEQAPNMAEPR |
| | | | | | | GPVDH |
| | | | | | | GVQIR |
| 2364.1184 | 2364.1904 | .072 | 30 | 1 | 21 | MEQAPNMAEPR |
| | | | | | | GPVDH |
| | | | | | | GVQIR |
| 2392.1562 | 2392.2158 | .0596 | 25 | 294 | 315 | STPDLLRDQQEA |
| | | | | | | APPGS |
| | | | | | | VDHMK |
| 2497.2505 | 2497.2788 | .0283 | 11 | 301 | 323 | DQQEAAPPGSV |
| | | | | | | DHMKAT |
| | | | | | | IYGILR |

| | |
|---|---|
| CENPE variant protein [Homo sapiens] | gi\|68533047 |

Peptide Information

| | Obsrv. | | | Start | End | |
|---|---|---|---|---|---|---|
| Calc. Mass | Mass | ±da | ±ppm | Seq. | Seq. | Sequence |
| 860.4472 | 860.3981 | −.0491 | −57 | 1113 | 1119 | SQQLQEK |
| 870.5593 | 870.476 | −.0833 | −96 | 154 | 160 | MKPLIIR |
| 930.489 | 930.4792 | −.0098 | −11 | 1493 | 1500 | QLEAINDK |
| 1084.5707 | 1084.5876 | .0169 | 16 | 635 | 643 | MKELATTYK |
| 1084.5707 | 1084.5876 | .0169 | 16 | 635 | 643 | MKELATTYK |
| 1141.5485 | 1141.5925 | .044 | 39 | 1737 | 1746 | DLEHSNDALK |
| 1142.6641 | 1142.6134 | −.0507 | −44 | 247 | 258 | AAQTGAAGVRLK |
| 1158.5459 | 1158.472 | −.0739 | −64 | 1857 | 1865 | LHENLEEMK |
| 1158.5459 | 1158.472 | −.0739 | −64 | 1857 | 1865 | LHENLEEMK |
| 1177.5696 | 1177.6279 | .0583 | 50 | 551 | 560 | KDQENELSSK |
| 1268.6594 | 1268.674 | .0146 | 12 | 1712 | 1721 | EHQETIDKLR |
| 1373.7272 | 1373.7284 | .0012 | 1 | 1242 | 1253 | TAQIINTQDLEK |
| 1374.6682 | 1374.7214 | .0532 | 39 | 2483 | 2494 | QVTCENSPKSPK |
| 1401.6243 | 1401.7521 | .1278 | 91 | 2078 | 2088 | FEMDFIDEVEK |
| 1438.7649 | 1438.6984 | −.0665 | −46 | 2267 | 2278 | IRELENSLHEAK |
| 1509.802 | 1509.719 | −.083 | −55 | 233 | 246 | VSHLNLVDLAGSER |
| 1517.7264 | 1517.7161 | −.0103 | −7 | 1410 | 1422 | LQESHDEMKSVAK |
| 1528.7853 | 1528.6842 | −.1011 | −66 | 747 | 759 | SEELHIITSEKDK |
| 1528.7853 | 1528.6842 | −.1011 | −66 | 747 | 759 | SEELHIITSEKDK |
| 1534.7748 | 1534.7842 | .0094 | 6 | 2205 | 2217 | SATISKEWEQDLK |
| 1566.801 | 1566.7755 | −.0255 | −16 | 1168 | 1180 | LNENYEEVKSITK |
| 1587.7869 | 1587.7997 | .0128 | 8 | 1857 | 1869 | LHENLEEMKSV |
| | | | | | | MK |
| 1591.7744 | 1591.7181 | −.0563 | −35 | 1784 | 1797 | LSNMQKDLENS |
| | | | | | | NAK |
| 1667.885 | 1667.8221 | −.0629 | −38 | 1447 | 1460 | EIVAKHLETEEE |
| | | | | | | LK |
| 1684.948 | 1684.9039 | −.0441 | −26 | 2294 | 2308 | ELEVTNDIIAKLQ |
| | | | | | | AK |
| 1684.948 | 1684.9039 | −.0441 | −26 | 2294 | 2308 | ELEVTNDIIAKLQ |
| | | | | | | AK |
| 1712.7949 | 1712.8146 | .0197 | 12 | 2151 | 2163 | EMTQFLEEWLN |
| | | | | | | TR |
| 2378.1255 | 2378.207 | .0815 | 34 | 134 | 153 | VSYMEIYNETIT |
| | | | | | | DLLCGT |
| | | | | | | QK |
| 2378.1255 | 2378.207 | .0815 | 34 | 134 | 153 | VSYMEIYNETIT |
| | | | | | | DLLCGT |
| | | | | | | QK |
| 2392.2693 | 2392.2158 | −.0535 | −22 | 233 | 256 | VSHLNLVDLAGS |
| | | | | | | ERAAQ |
| | | | | | | TGAAGVR |
| 2480.1213 | 2480.2493 | .128 | 52 | 783 | 803 | DDLATTQSNYK |
| | | | | | | STDQEF |
| | | | | | | QNFK |
| 2480.1213 | 2480.2493 | .128 | 52 | 783 | 803 | DDLATTQSNYK |
| | | | | | | STDQEF |
| | | | | | | QNFK |

| | |
|---|---|
| hCG1997007 [Homo sapiens] | gi\|119620152 |

Peptide Information

| Calc. | Obsrv. | | | Start | End | |
|---|---|---|---|---|---|---|
| Mass | Mass | ±da | ±ppm | Seq. | Seq. | Sequence |
| 1141.6266 | 1141.5925 | −.0341 | −30 | 8 | 16 | FHLAPW |
| | | | | | | SRK |
| 1161.6991 | 1161.6335 | −.0656 | −56 | 50 | 59 | FLSTPR |
| | | | | | | TLVK |
| 1438.7339 | 1438.6984 | −.0355 | −25 | 4 | 15 | GPDRFHL |
| | | | | | | APWSR |
| 2378.2212 | 2378.207 | −.0142 | −6 | 17 | 40 | GPVGWA |
| | | | | | | PALSGI |
| | | | | | | PQQEN |
| | | | | | | AAGSSRK |
| 2378.2212 | 2378.207 | −.0142 | −6 | 16 | 39 | KGPVGWA |
| | | | | | | PALSGIP |
| | | | | | | QQENAAG |
| | | | | | | SSR |

| | |
|---|---|
| centromere protein E, 312 kDa, | gi\|119626574 |
| isoform CRA_a [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 860.4472 | 860.3981 | −.0491 | −57 | 1133 | 1139 | SQQLQEK |
| 870.5593 | 870.476 | −.0833 | −96 | 149 | 155 | MKPLIIR |
| 930.489 | 930.4792 | −.0098 | −11 | 1513 | 1520 | QLEAINDK |
| 1084.5707 | 1084.5876 | .0169 | 16 | 655 | 663 | MKELATTYK |
| 1084.5707 | 1084.5876 | .0169 | 16 | 655 | 663 | MKELATTYK |
| 1125.5898 | 1125.6086 | .0188 | 17 | 2027 | 2035 | LHESLEEIR |
| 1141.5485 | 1141.5925 | .044 | 39 | 1757 | 1766 | DLEHSNDALK |
| 1142.6641 | 1142.6134 | −.0507 | −44 | 242 | 253 | AAQTGAAGVRLK |
| 1158.5459 | 1158.472 | −.0739 | −64 | 1877 | 1885 | LHENLEEMK |
| 1158.5459 | 1158.472 | −.0739 | −64 | 1877 | 1885 | LHENLEEMK |
| 1268.6594 | 1268.674 | .0146 | 12 | 1732 | 1741 | EHQETIDKLR |
| 1373.7272 | 1373.7284 | .0012 | 1 | 1262 | 1273 | TAQIINTQDLEK |
| 1374.6682 | 1374.7214 | .0532 | 39 | 2563 | 2574 | QVTCENSPKSPK |
| 1401.6243 | 1401.7521 | .1278 | 91 | 2158 | 2168 | FEMDFIDEVEK |
| 1438.7649 | 1438.6984 | −.0665 | −46 | 2347 | 2358 | IRELENSLHEAK |
| 1509.802 | 1509.719 | −.083 | −55 | 228 | 241 | VSHLNLVDLAGS |
| | | | | | | ER |
| 1517.7264 | 1517.7161 | −.0103 | −7 | 1430 | 1442 | LQESHDEMKSV |
| | | | | | | AK |
| 1528.7853 | 1528.6842 | −.1011 | −66 | 767 | 779 | SEELHIITSEKDK |
| 1528.7853 | 1528.6842 | −.1011 | −66 | 767 | 779 | SEELHIITSEKDK |
| 1534.7748 | 1534.7842 | .0094 | 6 | 2285 | 2297 | SATISKEWEQDLK |
| | 1534.7842 | | | | | |
| 1566.801 | 1566.7755 | −.0255 | −16 | 1188 | 1200 | LNENYEEVKSITK |
| 1587.7869 | 1587.7997 | .0128 | 8 | 1877 | 1889 | LHENLEEMKSV |
| | | | | | | MK |
| 1591.7744 | 1591.7181 | −.0563 | −35 | 1804 | 1817 | LSNMQKDLENS |
| | | | | | | NAK |
| 1667.885 | 1667.8221 | −.0629 | −38 | 1467 | 1480 | EIVAKHLETEEE |
| | | | | | | LK |
| 1684.948 | 1684.9039 | −.0441 | −26 | 2374 | 2388 | ELEVTNDIIAKLQ |
| | | | | | | AK |
| 1684.948 | 1684.9039 | −.0441 | −26 | 2374 | 2388 | ELEVTNDIIAKLQ |
| | | | | | | AK |
| 1712.7949 | 1712.8146 | .0197 | 12 | 2231 | 2243 | EMTQFLEEWLN |
| | | | | | | TR |
| 2378.1255 | 2378.207 | .0815 | 34 | 129 | 148 | VSYMEIYNETIT |
| | | | | | | DLLCGT |
| | | | | | | QK |
| 2378.1255 | 2378.207 | .0815 | 34 | 129 | 148 | VSYMEIYNETIT |
| | | | | | | DLLCGT |
| | | | | | | QK |
| 2392.2693 | 2392.2158 | −.0535 | −22 | 228 | 251 | VSHLNLVDLAGS |
| | | | | | | ERAAQ |
| | | | | | | TGAAGVR |
| 2480.1213 | 2480.2493 | .128 | 52 | 803 | 823 | DDLATTQSNYK |
| | | | | | | STDQEF |
| | | | | | | QNFK |
| 2480.1213 | 2480.2493 | .128 | 52 | 803 | 823 | DDLATTQSNYK |
| | | | | | | STDQEF QNFK |

| | |
|---|---|
| centromere protein E, 312 kDa, isoform CRA_b | gi\|119626575 |
| [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 860.4472 | 860.3981 | −.0491 | −57 | 1133 | 1139 | SQQLQEK |
| 870.5593 | 870.476 | −.0833 | −96 | 149 | 155 | MKPLIIR |
| 930.489 | 930.4792 | −.0098 | −11 | 1513 | 1520 | QLEAINDK |
| 1084.5707 | 1084.5876 | .0169 | 16 | 655 | 663 | MKELATTYK |
| 1084.5707 | 1084.5876 | .0169 | 16 | 655 | 663 | MKELATTYK |
| 1125.5898 | 1125.6086 | .0188 | 17 | 2027 | 2035 | LHESLEEIR |
| 1141.5485 | 1141.5925 | .044 | 39 | 1757 | 1766 | DLEHSNDALK |
| 1142.6641 | 1142.6134 | −.0507 | −44 | 242 | 253 | AAQTGAAGVRLK |
| 1158.5459 | 1158.472 | −.0739 | −64 | 1877 | 1885 | LHENLEEMK |
| 1158.5459 | 1158.472 | −.0739 | −64 | 1877 | 1885 | LHENLEEMK |
| 1268.6594 | 1268.674 | .0146 | 12 | 1732 | 1741 | EHQETIDKLR |
| 1373.7272 | 1373.7284 | .0012 | 1 | 1262 | 1273 | TAQIINTQDLEK |
| 1374.6682 | 1374.7214 | .0532 | 39 | 2562 | 2573 | QVTCENSPKSPK |
| 1401.6243 | 1401.7521 | .1278 | 91 | 2157 | 2167 | FEMDFIDEVEK |
| 1438.7649 | 1438.6984 | −.0665 | −46 | 2346 | 2357 | IRELENSLHEAK |
| 1509.802 | 1509.719 | −.083 | −55 | 228 | 241 | VSHLNLVDLAGS |
| | | | | | | ER |
| 1517.7264 | 1517.7161 | −.0103 | −7 | 1430 | 1442 | LQESHDEMKSV |
| | | | | | | AK |
| 1528.7853 | 1528.6842 | −.1011 | −66 | 767 | 779 | SEELHIITSEKDK |
| | 1528.6842 | | | | | |
| 1528.7853 | 1528.6842 | −.1011 | −66 | 767 | 779 | SEELHIITSEKDK |
| 1534.7748 | 1534.7842 | .0094 | 6 | 2284 | 2296 | SATISKEWEQDLK |
| 1566.801 | 1566.7755 | −.0255 | −16 | 1188 | 1200 | LNENYEEVKSITK |
| 1587.7869 | 1587.7997 | .0128 | 8 | 1877 | 1889 | LHENLEEMKSV |
| | | | | | | MK |
| 1591.7744 | 1591.7181 | −.0563 | −35 | 1804 | 1817 | LSNMQKDLENS |
| | | | | | | NAK |
| 1667.885 | 1667.8221 | −.0629 | −38 | 1467 | 1480 | EIVAKHLETEEE |
| | | | | | | LK |
| 1684.948 | 1684.9039 | −.0441 | −26 | 2373 | 2387 | ELEVTNDIIAKLQ |
| | | | | | | AK |
| 1684.948 | 1684.9039 | −.0441 | −26 | 2373 | 2387 | ELEVTNDIIAKLQ |
| | | | | | | AK |
| 1712.7949 | 1712.8146 | .0197 | 12 | 2230 | 2242 | EMTQFLEEWLN |
| | | | | | | TR |
| 2378.1255 | 2378.207 | .0815 | 34 | 129 | 148 | VSYMEIYNETIT |
| | | | | | | DLLCGT |
| | | | | | | QK |
| 2378.1255 | 2378.207 | .0815 | 34 | 129 | 148 | VSYMEIYNETIT |
| | | | | | | DLLCGT |
| | | | | | | QK |
| 2392.2693 | 2392.2158 | −.0535 | −22 | 228 | 251 | VSHLNLVDLAGS |
| | | | | | | ERAAQ |
| | | | | | | TGAAGVR |
| 2480.1213 | 2480.2493 | .128 | 52 | 803 | 823 | DDLATTQSNYK |
| | | | | | | STDQEF |
| | | | | | | QNFK |
| 2480.1213 | 2480.2493 | .128 | 52 | 803 | 823 | DDLATTQSNYK |
| | | | | | | STDQEF |
| | | | | | | QNFK |

| | |
|---|---|
| centromere-associated protein E [Homo sapiens] | gi\|71061468 |

Protein Group

| | |
|---|---|
| RecName: Full = Centromere-associated protein E; | gi\|160358869 |
| AltName: Full = Centromere protein E; | |
| Short = CENP-E; AltName: Full = Kinesin- | |
| related protein CENPE; Flags: | |
| Precursor | |
| centromere protein E, 312 kDa, isoform CRA_c [Homo | gi\|119626576 |
| sapiens] | |
| unnamed protein product [Homo sapiens] | gi\|311641337 |

Peptide Information

| | Obsrv. | | | Start | End | |
|---|---|---|---|---|---|---|
| Calc. Mass | Mass | ±da | ±ppm | Seq. | Seq. | Sequence |
| 860.4472 | 860.3981 | −.0491 | −57 | 1133 | 1139 | SQQLQEK |
| 870.5593 | 870.476 | −.0833 | −96 | 149 | 155 | MKPLIIR |
| 930.489 | 930.4792 | −.0098 | −11 | 1513 | 1520 | QLEAINDK |
| 1084.5707 | 1084.5876 | .0169 | 16 | 655 | 663 | MKELATTYK |
| 1084.5707 | 1084.5876 | .0169 | 16 | 655 | 663 | MKELATTYK |
| 1125.5898 | 1125.6086 | .0188 | 17 | 2027 | 2035 | LHESLEEIR |
| 1141.5485 | 1141.5925 | .044 | 39 | 1757 | 1766 | DLEHSNDALK |
| 1142.6641 | 1142.6134 | −.0507 | −44 | 242 | 253 | AAQTGAAGVRLK |
| | 1142.6134 | | | | | |
| 1158.5459 | 1158.472 | −.0739 | −64 | 1877 | 1885 | LHENLEEMK |
| 1158.5459 | 1158.472 | −.0739 | −64 | 1877 | 1885 | LHENLEEMK |
| 1268.6594 | 1268.674 | .0146 | 12 | 1732 | 1741 | EHQETIDKLR |
| 1373.7272 | 1373.7284 | .0012 | 1 | 1262 | 1273 | TAQIINTQDLEK |
| 1374.6682 | 1374.7214 | .0532 | 39 | 2599 | 2610 | QVTCENSPKSPK |
| 1401.6243 | 1401.7521 | .1278 | 91 | 2194 | 2204 | FEMDFIDEVEK |
| 1438.7649 | 1438.6984 | −.0665 | −46 | 2383 | 2394 | IRELENSLHEAK |
| 1509.802 | 1509.719 | −.083 | −55 | 228 | 241 | VSHLNLVDLAGS |
| | | | | | | ER |
| 1517.7264 | 1517.7161 | −.0103 | −7 | 1430 | 1442 | LQESHDEMKSV |
| | | | | | | AK |
| 1528.7853 | 1528.6842 | −.1011 | −66 | 767 | 779 | SEELHIITSEKDK |
| 1528.7853 | 1528.6842 | −.1011 | −66 | 767 | 779 | SEELHIITSEKDK |
| 1534.7748 | 1534.7842 | .0094 | 6 | 2321 | 2333 | SATISKEWEQDLK |
| 1566.801 | 1566.7755 | −.0255 | −16 | 1188 | 1200 | LNENYEEVKSITK |
| 1587.7869 | 1587.7997 | .0128 | 8 | 1877 | 1889 | LHENLEEMKSV |
| | | | | | | MK |
| 1591.7744 | 1591.7181 | −.0563 | −35 | 1804 | 1817 | LSNMQKDLENS |
| | | | | | | NAK |
| 1667.885 | 1667.8221 | −.0629 | −38 | 1467 | 1480 | EIVAKHLETEEE |
| | | | | | | LK |
| 1684.948 | 1684.9039 | −.0441 | −26 | 2410 | 2424 | ELEVTNDIIAKLQ |
| | | | | | | AK |
| 1684.948 | 1684.9039 | −.0441 | −26 | 2410 | 2424 | ELEVTNDIIAKLQ |
| | | | | | | AK |
| 1712.7949 | 1712.8146 | .0197 | 12 | 2267 | 2279 | EMTQFLEEWLN |
| | | | | | | TR |
| 2378.1255 | 2378.207 | .0815 | 34 | 129 | 148 | VSYMEIYNETIT |
| | | | | | | DLLCGT |
| | | | | | | QK |
| 2378.1255 | 2378.207 | .0815 | 34 | 129 | 148 | VSYMEIYNETIT |
| | | | | | | DLLCGT |
| | | | | | | QK |
| 2392.2693 | 2392.2158 | −.0535 | −22 | 228 | 251 | VSHLNLVDLAGS |
| | | | | | | ERAAQ |
| | | | | | | TGAAGVR |
| 2480.1213 | 2480.2493 | .128 | 52 | 803 | 823 | DDLATTQSNYK |
| | | | | | | STDQEF |
| | | | | | | QNFK |
| 2480.1213 | 2480.2493 | .128 | 52 | 803 | 823 | DDLATTQSNYK |
| | | | | | | STDQEF QNFK |

| | |
|---|---|
| sorbin and SH3 domain containing 1 [Homo sapiens] | gi\|55957967 |

Peptide Information

| | Obsrv. | | | Start | End | |
|---|---|---|---|---|---|---|
| Calc. Mass | Mass | ±da | ±ppm | Seq. | Seq. | Sequence |
| 1084.531 | 1084.5876 | .0566 | 52 | 333 | 341 | SIYEYQPGK |
| 1084.531 | 1084.5876 | .0566 | 52 | 333 | 341 | SIYEYQPGK |
| 1158.4984 | 1158.472 | −.0264 | −23 | 280 | 289 | SEMSYIDGEK |
| 1158.4984 | 1158.472 | −.0264 | −23 | 280 | 289 | SEMSYIDGEK |
| 1174.4933 | 1174.5114 | .0181 | 15 | 280 | 289 | SEMSYID |
| | | | | | | GEK |
| 1268.7573 | 1268.674 | −.0833 | −66 | 583 | 593 | AQTLKELP |
| | | | | | | LQK |
| 1297.6127 | 1297.6924 | .0797 | 61 | 26 | 36 | DMDLTKICTGK |
| 1373.6254 | 1373.7284 | .103 | 75 | 278 | 289 | SKSEMSYIDGEK |
| 1477.7645 | 1477.7496 | −.0149 | −10 | 554 | 566 | FGDLLNIDDTAKR |
| 1509.7584 | 1509.719 | −.0394 | −26 | 329 | 341 | AEPKSIYEYQPGK |
| 1546.6769 | 1546.7771 | .1002 | 65 | 308 | 320 | SSSERNDWEPP |
| | | | | | | DK |
| 1591.7671 | 1591.7181 | −.049 | −31 | 478 | 492 | RPSSSASTKDSE |
| | | | | | | SPR |
| 1712.849 | 1712.8146 | −.0344 | −20 | 263 | 277 | YSFSEDTKSPLS |
| | | | | | | VPR |
| 2480.1895 | 2480.2493 | .0598 | 24 | 384 | 404 | DLSLCQTELEAD |
| | | | | | | LEKMET |
| | | | | | | LNK |
| 2480.1895 | 2480.2493 | .0598 | 24 | 384 | 404 | DLSLCQTELEAD |
| | | | | | | LEKMET |
| | | | | | | LNK |
| 2497.0752 | 2497.2788 | .2036 | 82 | 1 | 25 | MSSECDGGSKA |
| | | | | | | VMNGL |
| | | | | | | APGSNGQDK |

| | |
|---|---|
| CDC20-like protein form 3 [Homo sapiens] | gi\|29603480 |

Peptide Information

| | Obsrv. | | | Start | End | |
|---|---|---|---|---|---|---|
| Calc. Mass | Mass | ±da | ±ppm | Seq. | Seq. | Sequence |
| 813.3672 | 813.4328 | .0656 | 81 | 145 | 151 | APHAMDR |
| 842.4843 | 842.47 | −.0143 | −17 | 5 | 11 | LERTAPR |
| 860.4625 | 860.3981 | −.0644 | −75 | 2 | 7 | EWKLER |
| 930.5255 | 930.4792 | −.0463 | −50 | 123 | 130 | EQLKTPSK |
| 1129.6438 | 1129.6481 | .0043 | 4 | 406 | 414 | ICSLIWLPK |
| 1534.7319 | 1534.7842 | .0523 | 34 | 427 | 439 | NDVTVWTCPTVSR |
| 1550.7268 | 1550.7622 | .0354 | 23 | 131 | 144 | GISETSNSALHFCK |
| 2378.1545 | 2378.207 | .0525 | 22 | 284 | 305 | EGTCLAVGTSEG |
| | | | | | | EVQLW |
| | | | | | | DVVTK |
| 2378.1545 | 2378.207 | .0525 | 22 | 284 | 305 | EGTCLAVGTSEG |
| | | | | | | EVQLW |
| | | | | | | DVVTK |
| 2392.2832 | 2392.2158 | −.0674 | −28 | 208 | 229 | SSGDINDSILQPE |
| | | | | | | VKIHIT |
| | | | | | | GLR |

| | |
|---|---|
| PREDICTED: putative zinc finger protein | gi\|310133350 |
| 705E-like [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 811.4785 | 811.3994 | −.0791 | −97 | 108 | 114 | QIHTKGK |
| 995.5156 | 995.5438 | .0282 | 28 | 236 | 244 | AFSLSSDLR |
| 1158.452 | 1158.472 | .02 | 17 | 199 | 207 | WYECDNSGK |
| 1158.452 | 1158.472 | .02 | 17 | 199 | 207 | WYECDNSGK |
| 1193.5579 | 1193.6301 | .0722 | 60 | 1 | 12 | MAAGLSANESSR |
| 1463.7974 | 1463.9257 | .1283 | 88 | 31 | 42 | THMISMHPIIRK |
| 1495.7872 | 1495.6833 | −.1039 | −69 | 31 | 42 | THMISMHPIIRK |
| 1495.7872 | 1495.6833 | −.1039 | −69 | 30 | 41 | KTHMISMHPIIR |
| 2364.0317 | 2364.1904 | .1587 | 67 | 115 | 132 | SYQCNLCEKAY |
| | | | | | | TNCFHLR |
| 2364.0317 | 2364.1904 | .1587 | 67 | 115 | 132 | SYQCNLCEKAY |
| | | | | | | TNCFHL R |

| | |
|---|---|
| sorting nexin-29 [Homo sapiens] | gi\|154091028 |

Protein Group

| | |
|---|---|
| RecName: Full = Sorting nexin-29 | gi\|156633630 |

Peptide Information

| | Obsrv. | | | Start | End | |
|---|---|---|---|---|---|---|
| Calc. Mass | Mass | ±da | ±ppm | Seq. | Seq. | Sequence |
| 813.4689 | 813.4328 | −.0361 | −44 | 410 | 416 | LSRGQPR |
| 860.4108 | 860.3981 | −.0127 | −15 | 131 | 137 | VAEQEER |
| 1037.4867 | 1037.5292 | .0425 | 41 | 87 | 95 | QATVAMMNR |
| 1161.5382 | 1161.6335 | .0953 | 82 | 96 | 104 | KDELEEENR |
| 1180.6508 | 1180.5763 | −.0745 | −63 | 159 | 168 | YVGAVQMLKR |
| 1415.804 | 1415.7649 | −.0391 | −28 | 227 | 239 | ALVAKEALVSQMR |
| 1509.7843 | 1509.719 | −.0653 | −43 | 352 | 363 | QLQNYLRSVMNK |
| 1544.8506 | 1544.7524 | −.0982 | −64 | 364 | 377 | VIQMVPEFAASPKK |
| 1555.7533 | 1555.8049 | .0516 | 33 | 108 | 121 | NLLDGEMEHSAA |
| | | | | | | LR |

| | |
|---|---|
| hCG1797938 [Homo sapiens] | gi\|119632137 |

Peptide Information

| | Obsrv. | | | Start | End | |
|---|---|---|---|---|---|---|
| Calc. Mass | Mass | ±da | ±ppm | Seq. | Seq. | Sequence |
| 1098.5651 | 1098.6075 | .0424 | 39 | 103 | 111 | HRSDIDLSR |
| 1374.6907 | 1374.7214 | .0307 | 22 | 153 | 167 | NGSGKVAGAGLA |
| | | | | | | CGR |
| 1685.0109 | 1684.9039 | −.107 | −64 | 85 | 98 | KKPTENVLIFLINR |
| 1685.0109 | 1684.9039 | −.107 | −64 | 85 | 98 | KKPTENVLIFLINR |
| 1698.7727 | 1698.9124 | .1397 | 82 | 127 | 141 | EACFPGIGGCAEF |
| | | | | | | KR |
| 1712.9078 | 1712.8146 | −.0932 | −54 | 2 | 16 | TSLGAEPIRPWDS |
| | | | | | | RK |
| 2480.1409 | 2480.2493 | .1084 | 44 | 46 | 65 | LCCFTEVGWSQH |
| | | | | | | IMDKE |
| | | | | | | VPK |
| 2480.1409 | 2480.2493 | .1084 | 44 | 46 | 65 | LCCFTEVGWSQH |
| | | | | | | IMDKE |
| | | | | | | VPK |

| | | | |
|---|---|---|---|
| | 42/B15 | | Instr./Gel |
| | | | Origin |
| | [1] Sample Project | | Instrument |
| | 20110503 | | Sample |
| | | | Name |
| Protein Name - | KH9 (SEQ ID NO. KH9) | Accession No. | |

| | |
|---|---|
| unnamed protein product [Homo sapiens] | gi\|1335098 |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4381 | −.0018 | −2 | 349 | 355 | LHIMAGR |
| 828.4111 | 828.4168 | .0057 | 7 | 55 | 60 | SHKWDR |
| 1070.5742 | 1070.5767 | .0025 | 2 | 191 | 199 | GEVPPRYPR |
| 1141.5889 | 1141.5946 | .0057 | 5 | 310 | 320 | GGYTLVSGYPK |
| 1142.6317 | 1142.5745 | −.0572 | −50 | 80 | 89 | QGHNSVFLIK |
| 1158.4707 | 1158.4755 | .0048 | 4 | 203 | 211 | DYFMPCPGR |
| 1158.4707 | 1158.4755 | .0048 | 4 | 203 | 211 | DYFMPCPGR |
| 1220.606 | 1220.6096 | .0036 | 3 | 69 | 79 | NFPSPVDAAFR |
| 1220.606 | 1220.6096 | .0036 | 3 | 69 | 79 | NFPSPVDAAFR |
| 1268.6747 | 1268.6786 | .0039 | 3 | 186 | 196 | FDPVRGEVPPR |
| 1297.6899 | 1297.6948 | .0049 | 4 | 310 | 321 | GGYTLVSGYPKR |
| 1495.6787 | 1495.6799 | .0012 | 1 | 175 | 185 | YYCFQGNQFLR |
| 1495.6787 | 1495.6799 | .0012 | 1 | 175 | 185 | YYCFQGNQFLR |
| 1512.6722 | 1512.7058 | .0336 | 22 | 200 | 211 | DVRDYFMPCPGR |
| 1528.6672 | 1528.6815 | .0143 | 9 | 200 | 211 | DVRDYFMPCPGR |
| 1528.6672 | 1528.6815 | .0143 | 9 | 200 | 211 | DVRDYFMPCPGR |
| 1534.7802 | 1534.7838 | .0036 | 2 | 67 | 79 | WKNFPSPVDAAFR |
| 1712.7698 | 1712.8024 | .0326 | 19 | 129 | 143 | GECQAEGVLFFQ |
| | | | | | | GDR |
| 2364.1653 | 2364.1797 | .0144 | 6 | 108 | 128 | LLQDEFPGIPSPL |
| | | | | | | DAAVE |
| | | | | | | CHR |
| 2364.1653 | 2364.1797 | .0144 | 6 | 108 | 128 | LLQDEFPGIPSPL |
| | | | | | | DAAVE |
| | | | | | | CHR |
| 2498.2344 | 2498.2512 | .0168 | 7 | 325 | 348 | EVGTPHGIILDSV |
| | | | | | | DAAFIC |
| | | | | | | PGSSR |
| 2498.2344 | 2498.2512 | .0168 | 7 | 325 | 348 | EVGTPHGIILDSV |
| | | | | | | DAAFIC |
| | | | | | | PGSSR |
| 3282.5195 | 3282.6226 | .1031 | 31 | 389 | 418 | SLGPNSCSANGP |
| | | | | | | GLYLIH |
| | | | | | | GPNLYCYSDVEK |

| | |
|---|---|
| hemopexin, isoform CRA_b [Homo sapiens] | gi\|119589125 |

Protein Group

| | |
|---|---|
| RecName: Full = Hemopexin; AltName: | gi\|1708182 |
| Full = Beta-1B- | |
| glycoprotein; Flags: | |
| Precursor | |
| hemopexin [Homo sapiens] | gi\|184497 |
| hemopexin precursor [Homo sapiens] | gi\|11321561 |
| hemopexin precursor [Homo sapiens] | gi\|386789 |
| unnamed protein product [Homo sapiens] | gi\|189053897 |
| unnamed protein product [Homo sapiens] | gi\|80747621 |

Peptide Information

| | Obsrv. | | | Start | End | |
|---|---|---|---|---|---|---|
| Calc. Mass | Mass | ±da | ±ppm | Seq. | Seq. | Sequence |
| 813.4399 | 813.4381 | −.0018 | −2 | 372 | 378 | LHIMAGR |
| 828.4111 | 828.4168 | .0057 | 7 | 78 | 83 | SHKWDR |
| 1070.5742 | 1070.5767 | .0025 | 2 | 214 | 222 | GEVPPRYPR |
| 1141.5889 | 1141.5946 | .0057 | 5 | 333 | 343 | GGYTLVSGYPK |
| 1142.6317 | 1142.5745 | −.0572 | −50 | 103 | 112 | QGHNSVFLIK |
| 1158.4707 | 1158.4755 | .0048 | 4 | 226 | 234 | DYFMPCPGR |
| 1158.4707 | 1158.4755 | .0048 | 4 | 226 | 234 | DYFMPCPGR |
| 1220.606 | 1220.6096 | .0036 | 3 | 92 | 102 | NFPSPVDAAFR |
| 1220.606 | 1220.6096 | .0036 | 3 | 92 | 102 | NFPSPVDAAFR |
| 1268.6747 | 1268.6786 | .0039 | 3 | 209 | 219 | FDPVRGEVPPR |
| 1297.6899 | 1297.6948 | .0049 | 4 | 333 | 344 | GGYTLVSGYPKR |
| 1495.6787 | 1495.6799 | .0012 | 1 | 198 | 208 | YYCFQGNQFLR |
| 1495.6787 | 1495.6799 | .0012 | 1 | 198 | 208 | YYCFQGNQFLR |
| 1512.6722 | 1512.7058 | .0336 | 22 | 223 | 234 | DVRDYFMPCPGR |
| 1528.6672 | 1528.6815 | .0143 | 9 | 223 | 234 | DVRDYFMPCPGR |
| 1528.6672 | 1528.6815 | .0143 | 9 | 223 | 234 | DVRDYFMPCPGR |
| 1534.7802 | 1534.7838 | .0036 | 2 | 90 | 102 | WKNFPSPVDAAFR |
| 1712.7698 | 1712.8024 | .0326 | 19 | 152 | 166 | GECQAEGVLFFQGDR |
| | 1712.8024 | | | | | |
| 2364.1653 | 2364.1797 | .0144 | 6 | 131 | 151 | LLQDEFPGI |
| | | | | | | PSPLDAAVE CHR |
| 2364.1653 | 2364.1797 | .0144 | 6 | 131 | 151 | LLQDEFPGI |
| | | | | | | PSPLDAAVE CHR |
| 2498.2344 | 2498.2512 | .0168 | 7 | 348 | 371 | EVGTPHGIIL |
| | | | | | | DSVDAAFIC |
| | | | | | | PGSSR |
| 2498.2344 | 2498.2512 | .0168 | 7 | 348 | 371 | EVGTPHGIIL |
| | | | | | | DSVDAAFIC |
| | | | | | | PGSSR |
| 3282.5195 | 3282.6226 | .1031 | 31 | 412 | 441 | SLGPNSCSA |
| | | | | | | NGPGLYLIH |
| | | | | | | GPNLYCYSDVEK |

| | |
|---|---|
| HPX protein [Homo sapiens] | gi\|13529281 |

Peptide Information

| | Obsrv. | | | Start | End | |
|---|---|---|---|---|---|---|
| Calc. Mass | Mass | ±da | ±ppm | Seq. | Seq. | Sequence |
| 828.4111 | 828.4168 | .0057 | 7 | 78 | 83 | SHKWDR |
| 1070.5742 | 1070.5767 | .0025 | 2 | 214 | 222 | GEVPPRYPR |
| 1142.6317 | 1142.5745 | −.0572 | −50 | 103 | 112 | QGHNSVFLIK |
| 1158.4707 | 1158.4755 | .0048 | 4 | 226 | 234 | DYFMPCPGR |
| 1158.4707 | 1158.4755 | .0048 | 4 | 226 | 234 | DYFMPCPGR |
| 1220.606 | 1220.6096 | .0036 | 3 | 92 | 102 | NFPSPVDAAFR |
| 1220.606 | 1220.6096 | .0036 | 3 | 92 | 102 | NFPSPVDAAFR |
| 1268.6747 | 1268.6786 | .0039 | 3 | 209 | 219 | FDPVRGEVPPR |
| 1495.6787 | 1495.6799 | .0012 | 1 | 198 | 208 | YYCFQGNQFLR |
| 1495.6787 | 1495.6799 | .0012 | 1 | 198 | 208 | YYCFQGNQFLR |
| 1512.6722 | 1512.7058 | .0336 | 22 | 223 | 234 | DVRDYFMPCPGR |
| 1528.6672 | 1528.6815 | .0143 | 9 | 223 | 234 | DVRDYFMPCPGR |
| 1528.6672 | 1528.6815 | .0143 | 9 | 223 | 234 | DVRDYFMPCPGR |
| 1534.7802 | 1534.7838 | .0036 | 2 | 90 | 102 | WKNFPSPVDAAFR |
| 1712.7698 | 1712.8024 | .0326 | 19 | 152 | 166 | GECQAEGVLFFQ |
| | | | | | | GDR |
| 2364.1653 | 2364.1797 | .0144 | 6 | 131 | 151 | LLQDEFPGIPSPL |
| | | | | | | DAAVE |
| | | | | | | CHR |
| 2364.1653 | 2364.1797 | .0144 | 6 | 131 | 151 | LLQDEFPGIPSPL |
| | | | | | | DAAVE |
| | | | | | | CHR |

| | |
|---|---|
| hemopexin, isoform CRA_d [Homo sapiens] | gi\|119589127 |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4381 | −.0018 | −2 | 301 | 307 | LHIMAGR |
| 828.4111 | 828.4168 | .0057 | 7 | 78 | 83 | SHKWDR |
| 1141.5889 | 1141.5946 | .0057 | 5 | 262 | 272 | GGYTLVSGYPK |
| 1142.6317 | 1142.5745 | −.0572 | −50 | 103 | 112 | QGHNSVFLIK |
| 1220.606 | 1220.6096 | .0036 | 3 | 92 | 102 | NFPSPVDAAFR |
| 1220.606 | 1220.6096 | .0036 | 3 | 92 | 102 | NFPSPVDAAFR |
| 1297.6899 | 1297.6948 | .0049 | 4 | 262 | 273 | GGYTLVSGYPKR |
| 1534.7802 | 1534.7838 | .0036 | 2 | 90 | 102 | WKNFPSPVDAA |
| | | | | | | FR |
| 2364.1653 | 2364.1797 | .0144 | 6 | 131 | 151 | LLQDEFPGIPSP |
| | | | | | | LDAAVE |
| | | | | | | CHR |
| 2364.1653 | 2364.1797 | .0144 | 6 | 131 | 151 | LLQDEFPGIPSP |
| | | | | | | LDAAVE |
| | | | | | | CHR |
| 2498.2344 | 2498.2512 | .0168 | 7 | 277 | 300 | EVGTPHGIILDSV |
| | | | | | | DAAFIC |
| | | | | | | PGSSR |
| 2498.2344 | 2498.2512 | .0168 | 7 | 277 | 300 | EVGTPHGIILDSV |
| | | | | | | DAAFIC |
| | | | | | | PGSSR |
| 3282.5195 | 3282.6226 | .1031 | 31 | 341 | 370 | SLGPNSCSANGPGLYLIH |
| | | | | | | GPNLYCYSDVEK |

| | |
|---|---|
| hemopexin, isoform CRA_a [Homo sapiens] | gi\|119589124 |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4381 | −.0018 | −2 | 118 | 124 | LHIMAGR |
| 1141.5889 | 1141.5946 | .0057 | 5 | 79 | 89 | GGYTLVSGYPK |
| 1297.6899 | 1297.6948 | .0049 | 4 | 79 | 90 | GGYTLVSGYPKR |
| 2498.2344 | 2498.2512 | .0168 | 7 | 94 | 117 | EVGTPHGIILDSV |
| | | | | | | DAAFIC |
| | | | | | | PGSSR |
| 2498.2344 | 2498.2512 | .0168 | 7 | 94 | 117 | EVGTPHGIILDSV |
| | | | | | | DAAFIC |
| | | | | | | PGSSR |
| 3282.5195 | 3282.6226 | .1031 | 31 | 158 | 187 | SLGPNSCSANGP |
| | | | | | | GLYLIH |
| | | | | | | GPNLYCYSDVEK |

| | |
|---|---|
| hemopexin, isoform CRA_c [Homo sapiens] | gi\|119589126 |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4381 | −.0018 | −2 | 170 | 176 | LHIMAGR |
| 1141.5889 | 1141.5946 | .0057 | 5 | 131 | 141 | GGYTLVSGYPK |
| 1297.6899 | 1297.6948 | .0049 | 4 | 131 | 142 | GGYTLVSGYPKR |
| 2498.2344 | 2498.2512 | .0168 | 7 | 146 | 169 | EVGTPHGIILDSV |
| | | | | | | DAAFIC |
| | | | | | | PGSSR |
| 2498.2344 | 2498.2512 | .0168 | 7 | 146 | 169 | EVGTPHGIILDSV |
| | | | | | | DAAFIC |
| | | | | | | PGSSR |
| 3282.5195 | 3282.6226 | .1031 | 31 | 210 | 239 | SLGPNSCSANGP |
| | | | | | | GLYLIH |
| | | | | | | GPNLYCYSDVEK |

| | |
|---|---|
| hemopexin | gi\|226337 |

Peptide Information

| | | | | Start | End | |
|---|---|---|---|---|---|---|
| Calc. Mass | Obsrv. Mass | ±da | ±ppm | Seq. | Seq. | Sequence |
| 1141.5889 | 1141.5946 | .0057 | 5 | 78 | 88 | GGYTLVSGYPK |
| 1297.6899 | 1297.6948 | .0049 | 4 | 78 | 89 | GGYTLVSGYPKR |
| 2498.2344 | 2498.2512 | .0168 | 7 | 93 | 116 | EVGTPHGIILDSV |
| | | | | | | DAAFIC |
| | | | | | | PGSSR |
| 2498.2344 | 2498.2512 | .0168 | 7 | 93 | 116 | EVGTPHGIILDSV |
| | | | | | | DAAFIC |
| | | | | | | PGSSR |

| | |
|---|---|
| unnamed protein product [Homo sapiens] | gi\|221044726 |

Peptide Information

| | | | | Start | End | |
|---|---|---|---|---|---|---|
| Calc. Mass | Obsrv. Mass | ±da | ±ppm | Seq. | Seq. | Sequence |
| 828.4111 | 828.416 | .0057 | 7 | 7 | 83 | SHKWDR |
| 1142.6317 | 1142.574 | −.0572 | — | 10 | 11 | QGHNSVFLIK |
| 1220.606 | 1220.609 | .0036 | 3 | 9 | 10 | NFPSPVDAAFR |
| 1220.606 | 1220.609 | .0036 | 3 | 9 | 10 | NFPSPVDAAFR |

| | |
|---|---|
| cytotoxic ligand TRAIL receptor [Homo sapiens] | gi\|1945072 |

Protein Group

| | |
|---|---|
| tumor necrosis factor receptor superfamily, member | gi\|119584040 |
| 10a | |
| [Homo sapiens] | |
| unnamed protein product [Homo sapiens] | gi\|158256034 |
| unnamed protein product [Homo sapiens] | gi\|112186609 |
| unnamed protein product [Homo sapiens] | gi\|29563434 |

Peptide Information

| | Obsrv. | | | Start | End | |
|---|---|---|---|---|---|---|
| Calc. Mass | Mass | ±da | ±ppm | Seq. | Seq. | Sequence |
| 828.4686 | 828.4168 | −.0518 | −63 | 81 | 87 | EASPRLR |
| 841.4639 | 841.4127 | −.0512 | −61 | 45 | 52 | IEPRGGGR |
| 860.4737 | 860.4071 | −.0666 | −77 | 415 | 421 | WVNKTGR |
| 900.4355 | 900.4484 | .0129 | 14 | 435 | 441 | MEERHAK |
| 1268.5544 | 1268.6786 | .1242 | 98 | 207 | 217 | CSTGCPRGMVK |
| | 1268.6786 | | | | | |
| 1566.7655 | 1566.7692 | .0037 | 2 | 399 | 414 | AGTAGPGDALYAMLMK |
| 2364.2268 | 2364.1797 | −.0471 | −20 | 350 | 370 | RLLVPANG |
| | | | | | | ADPTETLMLF |
| | | | | | | FDK |
| 2364.2268 | 2364.1797 | −.0471 | −20 | 350 | 370 | RLLVPANG |
| | | | | | | ADPTETLMLF |
| | | | | | | FDK |
| 2392.2 | 2392.2051 | .0051 | 2 | 392 | 414 | NEIDVVRA |
| | | | | | | GTAGPGDALY |
| | | | | | | AMLMK |
| 3282.7229 | 3282.6226 | −.1003 | −31 | 2 | 36 | APPPARVHLGAFLAVTPN |
| | | | | | | PGSAASGTEAAAATPSK |

| | |
|---|---|
| hCG28655 [8 Homo sapiens] | gi\|119623000 |

Peptide Information

| | | | | Start | End | |
|---|---|---|---|---|---|---|
| Calc. Mass | Obsrv. Mass | ±da | ±ppm | Seq. | Seq. | Sequence |
| 828.3774 | 828.4168 | .0394 | 48 | 551 | 556 | QFLEYE |
| 841.4566 | 841.4127 | −.0439 | −52 | 268 | 274 | SPHIPYK |
| 995.4833 | 995.5543 | .071 | 71 | 176 | 183 | FSTWEPTK |
| 1070.5854 | 1070.5767 | −.0087 | −8 | 120 | 128 | HIHEHAKAK |
| 1220.6997 | 1220.6096 | −.0901 | −74 | 185 | 194 | ELELLKHNPK |
| 1220.6997 | 1220.6096 | −.0901 | −74 | 185 | 194 | ELELLKHNPK |
| 1229.7113 | 1229.8269 | .1156 | 94 | 27 | 38 | AFATIGVPARAR |
| 1325.7762 | 1325.8303 | .0541 | 41 | 431 | 441 | KLPIVACFHLK |
| 1544.8656 | 1544.744 | −.1216 | −79 | 2 | 16 | SVAPSSPHAALV |
| | | | | | | RPR |
| 1684.7418 | 1684.8979 | .1561 | 93 | 412 | 425 | IKCSGCHSYQES |
| | | | | | | TK |
| 1684.7418 | 1684.8979 | .1561 | 93 | 412 | 425 | IKCSGCHSYQES |
| | | | | | | TK |
| 1698.8374 | 1698.9072 | .0698 | 41 | 537 | 550 | DVLDSEGYLLFY |
| | | | | | | HK |
| 2378.1658 | 2378.1941 | .0283 | 12 | 453 | 473 | ITTYVSFPLELD |
| | | | | | | MTPFMA |
| | | | | | | SSK |
| 2378.1658 | 2378.1941 | .0283 | 12 | 453 | 473 | ITTYVSFPLELD |
| | | | | | | MTPFMA |
| | | | | | | SSK |

| | |
|---|---|
| chromosome 10 open reading frame 68 | gi\|168985097 |
| [Homo sapiens] | |

Protein Group

| | |
|---|---|
| C10orf68 protein [Homo sapiens] | gi\|115528720 |
| chromosome 10 open reading frame 68 [Homo sapiens] | gi\|168984617 |
| chromosome 10 open reading frame 68 [Homo sapiens] | gi\|168984251 |
| chromosome 10 open reading frame 68 [Homo sapiens] | gi\|168984530 |

Peptide Information

| | | | | Start | End | |
|---|---|---|---|---|---|---|
| Calc. Mass | Obsrv. Mass | ±da | ±ppm | Seq. | Seq. | Sequence |
| 800.3897 | 800.4257 | .036 | 45 | 116 | 122 | HQDSVSK |
| 807.4207 | 807.3802 | −.0405 | −50 | 236 | 242 | SESQTKK |
| 813.4213 | 813.4381 | .0168 | 21 | 390 | 396 | GHQKTDK |
| 828.4686 | 828.4168 | −.0518 | −63 | 164 | 170 | ITPGRER |
| 830.473 | 830.392 | −.081 | −98 | 382 | 389 | GSINAQLK |
| 832.3618 | 832.3807 | .0189 | 23 | 79 | 85 | HQDSMSK |
| 1161.5746 | 1161.6329 | .0583 | 50 | 303 | 312 | SKSLPETDER |
| 1174.5885 | 1174.5157 | −.0728 | −62 | 1 | 12 | MVPASASGEGLR |
| 1268.6846 | 1268.6786 | −.006 | −5 | 38 | 48 | LVVEHQESLSK |
| 1282.7729 | 1282.688 | −.0849 | −66 | 462 | 473 | DIIKGPISAQLK |
| 1534.6989 | 1534.7838 | .0849 | 55 | 79 | 91 | HQDSMSKSEMQ |
| | | | | | | VK |
| 1566.7694 | 1566.7692 | −.0002 | 0 | 513 | 525 | DMSVQRQEG |
| 1712.816 | 1712.8024 | −.0136 | −8 | 101 | 115 | IITHDEESGE |

| | |
|---|---|
| pre-progastrin releasing peptide [Homo sapiens] | gi\|306807 |

Peptide Information

| | | | | Start | End | |
|---|---|---|---|---|---|---|
| Calc. Mass | Obsrv. Mass | ±da | ±ppm | Seq. | Seq. | Sequence |
| 815.437 | 815.4804 | .0434 | 53 | 131 | 138 | LSAPGSQR |
| 841.4162 | 841.4127 | −.0035 | −4 | 142 | 148 | NPQLNQQ |
| 1125.5018 | 1125.6086 | .1068 | 95 | 54 | 64 | STGESSSVSER |
| 1510.7344 | 1510.6847 | −.0497 | −33 | 54 | 68 | STGESSSVSERG |
| | | | | | | SLK |
| 1712.933 | 1712.8024 | −.1306 | −76 | 77 | 91 | WEEAARNLLGLIE |
| | | | | | | AK |
| 2496.1274 | 2496.2483 | .1209 | 48 | 103 | 125 | ALGNQQPSWDSE |
| | | | | | | DSSNF |
| | | | | | | KDVGSK |

| | |
|---|---|
| hypothetical protein [Homo sapiens] | gi\|50949374 |

Peptide Information

| | | | | Start | End | |
|---|---|---|---|---|---|---|
| Calc. Mass | Obsrv. Mass | ±da | ±ppm | Seq. | Seq. | Sequence |
| 800.358 | 800.4257 | .0677 | 85 | 39 | 44 | DGRCHR |
| 856.525 | 856.4572 | −.0678 | −79 | 11 | 17 | LLETRPK |
| 1161.58 | 1161.6329 | .0529 | 46 | 3 | 10 | RDTLWWER |
| | 1161.6329 | | | | | |
| 1712.8789 | 1712.8024 | −.0765 | −45 | 71 | 87 | GFIAPCPGSPLG |
| | | | | | | SPSLR |
| 2480.2537 | 2480.248 | −.0057 | −2 | 64 | 87 | LPGPCSRGFIAP |
| | | | | | | CPGSPL |
| | | | | | | GSPSLR |
| 2480.2537 | 2480.248 | −.0057 | −2 | 64 | 87 | LPGPCSRGFIAP |
| | | | | | | CPGSPL |
| | | | | | | GSPSLR |

| | |
|---|---|
| immunoglobulin heavy chain variable region [Homo | gi\|3832687 |
| sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 800.3613 | 800.4257 | .0644 | 80 | 98 | 103 | YFDLWG |
| 830.4519 | 830.392 | −.0599 | −72 | 33 | 39 | GLEWIGR |
| 1220.6416 | 1220.6096 | −.032 | −26 | 87 | 97 | DSSITMVRGVR |
| 1220.6416 | 1220.6096 | −.032 | −26 | 87 | 97 | DSSITMVRGVR |
| 1544.7592 | 1544.744 | −.0152 | −10 | 40 | 53 | IYTSGSTNYNPSLK |
| 1684.8575 | 1684.8979 | .0404 | 24 | 56 | 70 | VTMSVDTSKNQF |
| | | | | | | SLK |
| 1684.8575 | 1684.8979 | .0404 | 24 | 56 | 70 | VTMSVDTSKNQF |
| | | | | | | SLK |

RecName: Full = Gastrin-releasing peptide; Short = GRP;
Contains: RecName: Full = Neuromedin-C; AltName:
Full = GRP-10; Flags: Precursor

Protein Group

| | |
|---|---|
| | gi\|308153451 |
| Gastrin-releasing peptide [Homo sapiens] | gi\|13325358 |
| gastrin-releasing peptide [Homo sapiens] | gi\|30582445 |
| gastrin-releasing peptide isoform 1 preproprotein | gi\|31542860 |
| [Homo sapiens] | |
| gastrin-releasing peptide, isoform CRA_a [Homo | gi\|119583495 |
| sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 815.437 | 815.4804 | .0434 | 53 | 131 | 138 | LSAPGSQR |
| 841.4162 | 841.4127 | −.0035 | −4 | 142 | 148 | NPQLNQQ |
| 1125.5018 | 1125.6086 | .1068 | 95 | 54 | 64 | STGESSSVSER |
| | 1125.6086 | | | | | |
| 1510.7344 | 1510.6847 | −.0497 | −33 | 54 | 68 | STGESSSVSERG |
| | | | | | | SLK |
| 1712.933 | 1712.8024 | −.1306 | −76 | 77 | 91 | WEEAARNLLGLI |
| | | | | | | EAK |
| 2496.1274 | 2496.2483 | .1209 | 48 | 103 | 125 | ALGNQQPSWDS |
| | | | | | | EDSSNF |
| | | | | | | KDVGSK |

| | |
|---|---|
| PREDICTED: putative zinc finger protein | gi\|310133350 |
| 705E-like [Homo sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 811.4785 | 811.4168 | −.0617 | −76 | 108 | 114 | QIHTKGK |
| 995.5156 | 995.5543 | .0387 | 39 | 236 | 244 | AFSLSSDLR |
| 1158.452 | 1158.4755 | .0235 | 20 | 199 | 207 | WYECDNSGK |
| 1158.452 | 1158.4755 | .0235 | 20 | 199 | 207 | WYECDNSGK |
| 1193.5579 | 1193.6306 | .0727 | 61 | 1 | 12 | MAAGLSANESSR |
| 1495.7872 | 1495.6799 | −.1073 | −72 | 31 | 42 | THMISMHPIIRK |
| 1495.7872 | 1495.6799 | −.1073 | −72 | 30 | 41 | KTHMISMHPIIR |
| 2364.0317 | 2364.1797 | .148 | 63 | 115 | 132 | SYQCNLCEKAYT |
| | | | | | | NCFHLR |
| 2364.0317 | 2364.1797 | .148 | 63 | 115 | 132 | SYQCNLCEKAYT |
| | | | | | | NCFHLR |

| | |
|---|---|
| tumor necrosis factor receptor superfamily member | gi\|226201419 |
| 10a | |
| [Homo sapiens] | |

Peptide Information

| | Obsrv. | | | Start | | |
|---|---|---|---|---|---|---|
| Calc. Mass | Mass | ±da | ±ppm | Seq. | End Seq. | Sequence |
| 828.4686 | 828.4168 | −.0518 | −63 | 80 | 86 | EASPRLR |
| 841.4639 | 841.4127 | −.0512 | −61 | 44 | 51 | IEPRGGGR |
| 860.4737 | 860.4071 | −.0666 | −77 | 414 | 420 | WVNKTGR |
| 900.4355 | 900.4484 | .0129 | 14 | 434 | 440 | MEERHAK |
| 1566.7655 | 1566.7692 | .0037 | 2 | 398 | 413 | AGTAGPGDALYA |
| | | | | | | MLMK |
| 2364.2268 | 2364.1797 | −.0471 | −20 | 349 | 369 | RLLVPANGADPT |
| | | | | | | ETLMLF |
| | | | | | | FDK |
| 2364.2268 | 2364.1797 | −.0471 | −20 | 349 | 369 | RLLVPANGADPT |
| | | | | | | ETLMLF |
| | | | | | | FDK |
| 2392.2 | 2392.2051 | .0051 | 2 | 391 | 413 | NEIDVVRAGTAGP |
| | | | | | | GDALY |
| | | | | | | AMLMK |
| 3282.7229 | 3282.6226 | −.1003 | −31 | 1 | 35 | APPPARVHLGAFL |
| | | | | | | AVTPN |
| | | | | | | PGSAASGTEAAA |
| | | | | | | ATPSK |

| | |
|---|---|
| Tumor necrosis factor receptor | gi\|15277527 |
| superfamily, member 10a [Homo sapiens] | |

Protein Group

| | |
|---|---|
| tumor necrosis factor receptor superfamily, member | gi\|117606478 |
| 10a | |
| [Homo sapiens] | |
| tumor necrosis factor receptor superfamily, member | gi\|30582651 |
| 10a | |
| [Homo sapiens] | |
| unnamed protein product [Homo sapiens] | gi\|238803394 |
| unnamed protein product [Homo sapiens] | gi\|218101354 |

Peptide Information

| | | | | Start | End | |
|---|---|---|---|---|---|---|
| Calc. Mass | Obsrv. Mass | ±da | ±ppm | Seq. | Seq. | Sequence |
| 828.4686 | 828.4168 | −.0518 | −63 | 81 | 87 | EASPRLR |
| 841.4639 | 841.4127 | −.0512 | −61 | 45 | 52 | IEPRGGGR |
| 860.4737 | 860.4071 | −.0666 | −77 | 415 | 421 | WVNKTGR |
| 900.4355 | 900.4484 | .0129 | 14 | 435 | 441 | MEERHAK |
| 1566.7655 | 1566.7692 | .0037 | 2 | 399 | 414 | AGTAGPGDALYA |
| | | | | | | MLMK |
| 2364.2268 | 2364.1797 | −.0471 | −20 | 350 | 370 | RLLVPANGADPT |
| | | | | | | ETLMLF |
| | | | | | | FDK |
| 2364.2268 | 2364.1797 | −.0471 | −20 | 350 | 370 | RLLVPANGADPT |
| | | | | | | ETLMLF |
| | | | | | | FDK |
| 2392.2 | 2392.2051 | .0051 | 2 | 392 | 414 | NEIDVVRAGTAGP |
| | | | | | | GDALY |
| | | | | | | AMLMK |
| 3282.7229 | 3282.6226 | −.1003 | −31 | 2 | 36 | APPPARVHLGAFLAVTPN |
| | | | | | | PGSAASGTEAAAATPSK |

| | |
|---|---|
| RecName: Full = NADH dehydrogenase [ubiquinone] 1 | gi\|6093488 |
| alpha subcomplex subunit 2; AltName: Full = Complex | |
| I-B8; Short = CI-B8; AltName: Full = NADH-ubiquinone | |
| oxidoreductase B8 subunit | |
| CD14 [Homo sapiens] | gi\|48146149 |

Protein Group

| | |
|---|---|
| NADH dehydrogenase (ubiquinone) 1 alpha | gi\|12539408 |
| subcomplex 2 [Homo sapiens] | |
| NADH dehydrogenase (ubiquinone) 1 alpha | gi\|13277540 |
| subcomplex, 2, 8 kDa [Homo sapiens] | |
| NADH dehydrogenase (ubiquinone) 1 alpha | gi\|119582435 |
| subcomplex, 2, 8 kDa [Homo sapiens] | |
| NADH dehydrogenase | gi\|4505355 |
| [ubiquinone] 1 alpha | |
| sapiens] | |
| NADH-ubiquinone oxidoreductase | gi\|2909862 |
| sapiens] | |

Peptide Information

| | | | | | End | |
|---|---|---|---|---|---|---|
| Calc. Mass | Obsrv. | ±da | ±ppm | Star Seq. | Seq. | Sequence |
| 807.4359 | 807.3802 | −.0557 | −69 | 35 | 40 | DFIEKR |
| 856.5363 | 856.4572 | −.0791 | −92 | 14 | 20 | LGLREIR |
| 870.5519 | 870.4911 | −.0608 | −70 | 9 | 17 | GVGAKLGLR |
| 930.5255 | 930.4865 | −.039 | −42 | 90 | 98 | ALENVLSGK |
| 3282.6389 | 3282.6226 | −.0163 | −5 | 69 | 98 | YAFGQETNVPLN |
| | | | | | | NFSAD |
| | | | | | | QVTRALENVLSGK |

| | |
|---|---|
| Chain A, Nmr Solution Structure Of Subunit | gi\|60593462 |
| B8 From | |
| Human Nadh-Ubiquinone Oxidoreductase | |
| Complex I | |
| (Ci-B8) | |

Peptide Information

| | Obsrv. | | | Start | | |
|---|---|---|---|---|---|---|
| Calc. Mass | Mass | ±da | ±ppm | Seq. | End Seq. | Sequence |
| 807.4359 | 807.3802 | −.0557 | −69 | 38 | 43 | DFIEKR |
| 856.5363 | 856.4572 | −.0791 | −92 | 17 | 23 | LGLREIR |
| 870.5519 | 870.4911 | −.0608 | −70 | 12 | 20 | GVGAKLGLR |
| 930.5255 | 930.4865 | −.039 | −42 | 93 | 101 | ALENVLSGK |
| 3282.6389 | 3282.6226 | −.0163 | −5 | 72 | 101 | YAFGQETNVPLN |
| | | | | | | NFSAD |
| | | | | | | QVTRALENVLSGK |

| | | | |
|---|---|---|---|
| | 43/B16 | | Instr./Gel |
| | | | Origin |
| | [1] Sample Project | | Instrument |
| | 20110503 | | Sample |
| | | | Name |
| Protein Name - | KH10 (SEQ ID NO. KH10) | Accession No. | |

| | |
|---|---|
| unnamed protein product [Homo sapiens] | gi\|311078934 |

Protein Group

| | |
|---|---|
| Chain A, Antithrombin-Anhydrothrombin-Heparin | gi\|52695555 |
| Ternary Complex Structure | |
| Chain A, Crystal Structure Of A Domain-Swapped | gi\|209870303 |
| Serpin Dimer | |
| Chain A, The Intact And Cleaved Human Antithrombin | gi\|999513 |
| Iii Complex As A Model For Serpin-Proteinase | |
| Interactions | |
| Chain B, Crystal Structure Of A Domain-Swapped | gi\|209870304 |
| Serpin Dimer | |
| Chain B, The Intact And Cleaved Human Antithrombin | gi\|157838181 |
| Iii Complex As A Model For Serpin-Proteinase | |
| Interactions | |
| Chain I, AntithrombinPENTASACCHARIDE | gi\|4389345 |
| COMPLEX | |
| Chain I, Biological Implications Of A 3 Angstroms | gi\|1000040 |
| Structure Of Dimeric Antithrombin | |
| Chain I, Crystal Structure At 2.6a Of The Ternary | gi\|31615461 |
| Complex Between Antithrombin, A P14-P8 Reactive | |
| Loop Peptide, And An Exogenous Tetrapeptide | |
| Chain I, Crystal Structure Of Antithrombin In The | gi\|37926953 |
| Pentasaccharide-Bound Intermediate | |
| State | |
| Chain I, Crystal Structure Of The Antithrombin-Factor | gi\|285803494 |
| Ixa-Pentasaccharide Complex | |
| Chain I, Implications For Function And Therapy Of A | gi\|6729898 |
| 2.9a Structure Of Binary-Complexed Antithrombin | |
| Chain I, Intermediate Structure Of Antithrombin Bound | gi\|209870630 |
| To The Natural Pentasaccharide | |
| Chain I, Plasma Alpha Antithrombin-Iii | gi\|8569312 |
| Chain I, Plasma Alpha Antithrombin-Iii | gi\|8569308 |
| And | |
| Pentasaccharide | |
| Chain I, Plasma Beta Antithrombin-Iii | gi\|8569310 |
| Chain I, Structure Of Dimeric Antithrombin Complexed | gi\|31615500 |
| With A P14-P9 Reactive Loop Peptide And An | |
| Exogenous Tripeptide | |
| Chain I, Structure Of Dimeric Antithrombin Complexed | gi\|55669596 |
| With A P14-P9 Reactive Loop Peptide And An | |
| Exogenous Tripeptide (Formyl-Norleucine-Lf) | |
| Chain I, The 2.6 A Structure Of Antithrombin Indicates | gi\|2392668 |
| A Conformational Change At The Heparin Binding Site | |
| Chain L, AntithrombinPENTASACCHARIDE | gi\|4389346 |
| COMPLEX | |
| Chain L, Biological Implications Of A 3 Angstroms | gi\|1000039 |
| Structure Of Dimeric Antithrombin | |
| Chain L, Crystal Structure At 2.6a Of The Ternary | gi\|31615460 |
| Complex Between Antithrombin, A P14-P8 Reactive | |
| Loop Peptide, And An Exogenous Tetrapeptide | |
| Chain L, Crystal Structure Of Antithrombin In The | gi\|37926954 |
| Pentasaccharide-Bound Intermediate | |
| State | |
| Chain L, Crystal Structure Of Antithrombin Variant | gi\|82408098 |
| S137aV317CT401C With Plasma Latent Antithrombin | |
| Chain L, Crystal Structure Of P13 Alanine Variant Of | gi\|48425164 |
| Antithrombin | |
| Chain L, Crystal Structure Of P14 Alanine Variant Of | gi\|150261272 |
| Antithrombin | |
| Chain L, Implications For Function And Therapy Of A | gi\|6729897 |
| 2.9a Structure Of Binary-Complexed Antithrombin | |
| Chain L, Intermediate Structure Of Antithrombin | gi\|209870631 |
| Bound | |
| To The Natural Pentasaccharide | |
| Chain L, N135q-S380c-Antithrombin-Iii | gi\|8569384 |
| Chain L, P14-Fluorescein-N135q-5380c-Antithrombin-Iii | gi\|8569386 |
| Chain L, Plasma Alpha Antithrombin-Iii | gi\|8569311 |
| Chain L, Plasma Alpha Antithrombin-Iii | gi\|8569307 |
| And | |
| Pentasaccharide | |
| Chain L, Plasma Beta Antithrombin-Iii | gi\|8569309 |
| Chain L, Structure Of Dimeric Antithrombin | gi\|31615499 |
| Complexed | |
| With A P14-P9 Reactive Loop Peptide And An | |
| Exogenous Tripeptide | |
| Chain L, Structure Of Dimeric Antithrombin | gi\|55669595 |
| Complexed | |
| With A P14-P9 Reactive Loop Peptide And An | |
| Exogenous Tripeptide (Formyl-Norleucine-Lf) | |
| Chain L, The 2.6 A Structure Of Antithrombin Indicates | gi\|2392667 |
| A Conformational Change At The Heparin Binding Site | |
| unnamed protein product [Homo sapiens] | gi\|311078924 |
| unnamed protein product [Homo sapiens] | gi\|311079000 |

Peptide Information

| | | | | Start | | |
|---|---|---|---|---|---|---|
| Calc. Mass | Obsrv. Mass | ±da | ±ppm | Seq. | End Seq. | Sequence |
| 850.4053 | 850.405 | −.0003 | 0 | 229 | 235 | FSPENTR |
| 850.4053 | 850.405 | −.0003 | 0 | 229 | 235 | FSPENTR |
| 978.5003 | 978.5024 | .0021 | 2 | 229 | 236 | FSPENTRK |
| 1175.6532 | 1175.6603 | .0071 | 6 | 394 | 403 | SLNPNRVTFK |
| 1211.642 | 1211.6508 | .0088 | 7 | 323 | 332 | FRIEDGFSLK |
| 1232.7263 | 1232.7316 | .0053 | 4 | 404 | 413 | ANRPFLVFIR |
| 1340.6635 | 1340.6824 | .0189 | 14 | 115 | 125 | TSDQIHFFFAK |
| 1340.6635 | 1340.6824 | .0189 | 14 | 115 | 125 | TSDQIHFFFAK |
| 1389.7559 | 1389.7552 | −.0007 | −1 | 414 | 425 | EVPLNTIIFMGR |
| 1405.7509 | 1405.7631 | .0122 | 9 | 414 | 425 | EVPLNTIIFMGR |
| 1405.7509 | 1405.7631 | .0122 | 9 | 414 | 425 | EVPLNTIIFMGR |
| 1437.7268 | 1437.7357 | .0089 | 6 | 1 | 13 | HGSPVDICTAKPR |
| 1441.6273 | 1441.6454 | .0181 | 13 | 14 | 24 | DIPMNPMCIYR |
| 1441.6273 | 1441.6454 | .0181 | 13 | 14 | 24 | DIPMNPMCIYR |
| 1674.8446 | 1674.8551 | .0105 | 6 | 170 | 183 | LQPLDFKENAEQ |
| | | | | | | SR |
| 1674.8446 | 1674.8551 | .0105 | 6 | 170 | 183 | LQPLDFKENAEQ |
| | | | | | | SR |
| 2202.1189 | 2202.1331 | .0142 | 6 | 351 | 370 | LPGIVAEGRDDL |
| | | | | | | YVSDAF |
| | | | | | | HK |
| 2291.1514 | 2291.175 | .0236 | 10 | 371 | 393 | AFLEVNEEGSEA |
| | | | | | | AASTAV VIAGR |

| | |
|---|---|
| RecName: Full = Antithrombin-III; Short = ATIII; | gi\|113936 |
| AltName: Full = Serpin C1; Flags: Precursor | |

Protein Group

| | |
|---|---|
| PRO0309 [Homo sapiens] | gi\|11493504 |
| antithrombin III [Homo sapiens] | gi\|179130 |
| antithrombin-III precursor [Homo sapiens] | gi\|4502261 |
| serine-cysteine proteinase inhibitor clade C member 1 | gi\|14326586 |
| [Homo sapiens] | |
| serpin peptidase inhibitor, clade C (antithrombin), | gi\|56204641 |
| member 1 [Homo sapiens] | |
| serpin peptidase inhibitor, clade C (antithrombin), | gi\|119611376 |
| member 1, isoform | |
| CRA_a [Homo | |
| sapiens] | |
| serpin peptidase inhibitor, clade C (antithrombin), | gi\|119611375 |
| member 1, isoform CRA_a [Homo | |
| sapiens] | |
| unnamed protein product [Homo sapiens] | gi\|311079002 |
| unnamed protein product [Homo sapiens] | gi\|311078958 |
| unnamed protein product [Homo sapiens] | gi\|189069199 |
| unnamed protein product [Homo sapiens] | gi\|311078948 |

Peptide Information

| | Obsrv. | | | Start | | |
|---|---|---|---|---|---|---|
| Calc. Mass | Mass | ±da | ±ppm | Seq. | End Seq. | Sequence |
| 850.4053 | 850.405 | −.0003 | 0 | 261 | 267 | FSPENTR |
| 850.4053 | 850.405 | −.0003 | 0 | 261 | 267 | FSPENTR |
| 978.5003 | 978.5024 | .0021 | 2 | 261 | 268 | FSPENTRK |
| 1175.6532 | 1175.6603 | .0071 | 6 | 426 | 435 | SLNPNRVTFK |
| 1211.642 | 1211.6508 | .0088 | 7 | 355 | 364 | FRIEDGFSLK |
| 1225.6423 | 1225.6396 | −.0027 | −2 | 2 | 13 | YSNVIGTVTSGK |
| 1232.7263 | 1232.7316 | .0053 | 4 | 436 | 445 | ANRPFLVFIR |
| 1340.6635 | 1340.6824 | .0189 | 14 | 147 | 157 | TSDQIHFFFAK |
| 1340.6635 | 1340.6824 | .0189 | 14 | 147 | 157 | TSDQIHFFFAK |
| 1389.7559 | 1389.7552 | −.0007 | −1 | 446 | 457 | EVPLNTIIFMGR |
| 1405.7509 | 1405.7631 | .0122 | 9 | 446 | 457 | EVPLNTIIFMGR |
| 1405.7509 | 1405.7631 | .0122 | 9 | 446 | 457 | EVPLNTIIFMGR |
| 1441.6273 | 1441.6454 | .0181 | 13 | 46 | 56 | DIPMNPMCIYR |
| 1441.6273 | 1441.6454 | .0181 | 13 | 46 | 56 | DIPMNPMCIYR |
| 1674.8446 | 1674.8551 | .0105 | 6 | 202 | 215 | LQPLDFKENAEQ |
| | | | | | | SR |
| 1674.8446 | 1674.8551 | .0105 | 6 | 202 | 215 | LQPLDFKENAEQ |
| | | | | | | SR |
| 2202.1189 | 2202.1331 | .0142 | 6 | 383 | 402 | LPGIVAEGRDDLY |
| | | | | | | VSDAF |
| | | | | | | HK |
| 2291.1514 | 2291.175 | .0236 | 10 | 403 | 425 | AFLEVNEEGSEA |
| | | | | | | AASTAV |
| | | | | | | VIAGR |

unnamed protein product [Homo sapiens]

Protein Group

| | |
|---|---|
| | gi\|311078998 |
| Chain A, Crystal Structure Of Native Antithrombin In | gi\|78100758 |
| Its | |
| Monomeric Form | |
| Chain B, Crystal Structure Of Native Antithrombin In | gi\|78100759 |
| Its | |
| Monomeric Form | |
| Chain C, Crystal Structure Of Native Antithrombin In | gi\|78100760 |
| Its | |
| Monomeric Form | |
| Chain C, Crystal Structure Of The Antithrombin-S195a | gi\|109157829 |
| Factor Xa-Pentasaccharide Complex | |
| Chain I, 2.1 Angstrom Structure Of A Nonproductive | gi\|116666783 |
| Complex Between Antithrombin, Synthetic Heparin | |
| Mimetic Sr123781 And Two S195a Thrombin | |
| Molecules | |
| Chain I, 2.5a Crystal Structure Of The | gi\|52695711 |
| Antithrombin-Thrombin-Heparin Ternary Complex | |
| Chain I, Crystal Structure Of Antithrombin Variant | gi\|82408097 |
| S137aV317CT401C With Plasma Latent Antithrombin | |
| Chain I, Crystal Structure Of | gi\|118137309 |
| Antithrombin-Iii | |
| Chain I, Crystal Structure Of P13 Alanine Variant Of | gi\|48425163 |
| Antithrombin | |
| Chain I, Crystal Structure Of P14 Alanine Variant Of | gi\|150261271 |
| Antithrombin | |
| Chain I, Crystal Structure Of The Antithrombin-S195a | gi\|109157826 |
| Factor Xa-Pentasaccharide Complex | |
| Chain I, N135q-S380c-Antithrombin-Iii | gi\|8569385 |
| Chain I, P14-Fluorescein-N135q-S380c-Antithrombin-Iii | gi\|8569387 |
| Chain L, Crystal Structure Of Antithrombin-Iii | gi\|118137310 |
| antithrombin | gi\|225767 |
| III | |
| antithrombin III [Homo sapiens] | gi\|179161 |
| antithrombin III [Homo sapiens] | |
| antithrombin III variant [Homo sapiens] | gi\|576554 |
| antithrombin III: ISOTYPE = toyama | gi\|228848 |
| antithrombin [Homo sapiens] | gi\|28907 |
| unnamed protein product [Homo sapiens] | gi\|311078996 |
| unnamed protein product [Homo sapiens] | gi\|311078994 |
| unnamed protein product [Homo sapiens] | gi\|311078992 |
| unnamed protein product [Homo sapiens] | gi\|311078990 |
| unnamed protein product [Homo sapiens] | gi\|311078988 |
| unnamed protein product [Homo sapiens] | gi\|311078986 |
| unnamed protein product [Homo sapiens] | gi\|311078984 |
| unnamed protein product [Homo sapiens] | gi\|311078970 |
| unnamed protein product [Homo sapiens] | gi\|311078968 |
| unnamed protein product [Homo sapiens] | gi\|57999350 |
| unnamed protein product [Homo sapiens] | gi\|311078966 |
| unnamed protein product [Homo sapiens] | gi\|311078964 |
| unnamed protein product [Homo sapiens] | gi\|311078962 |
| unnamed protein product [Homo sapiens] | gi\|311078960 |
| unnamed protein product [Homo sapiens] | gi\|311078956 |
| unnamed protein product [Homo sapiens] | gi\|311078954 |
| unnamed protein product [Homo sapiens] | gi\|311078952 |
| unnamed protein product [Homo sapiens] | gi\|311078950 |
| unnamed protein product [Homo sapiens] | gi\|311078946 |
| unnamed protein product [Homo sapiens] | gi\|311078944 |
| unnamed protein product [Homo sapiens] | gi\|311078942 |
| unnamed protein product [Homo sapiens] | gi\|311078940 |
| unnamed protein product [Homo sapiens] | gi\|311078938 |
| unnamed protein product [Homo sapiens] | gi\|311078936 |
| unnamed protein product [Homo sapiens] | gi\|311078932 |
| unnamed protein product [Homo sapiens] | gi\|311078930 |
| unnamed protein product [Homo sapiens] | gi\|311078928 |
| unnamed protein product [Homo sapiens] | gi\|311078926 |

Peptide Information

| | Obsrv. | | | Start | | |
|---|---|---|---|---|---|---|
| Calc. Mass | Mass | ±da | ±ppm | Seq. | End Seq. | Sequence |
| 1340.6635 | 1340.6824 | .0189 | 14 | 147 | 157 | TSDQIHFFFAK |
| 1405.7509 | 1405.7631 | .0122 | 9 | 446 | 457 | EVPLNTIIFMGR |
| 1674.8446 | 1674.8551 | .0105 | 6 | 202 | 215 | LQPLDFKENAEQ |
| | | | | | | SR |

Chain C, Solution Structure Of Human
Siga2

Protein Group

| | |
|---|---|
| | gi\|219109296 |
| Chain A, Crystal Structure Of A Staphylococcus | gi\|158430162 |
| Aureus | |
| Protein (Ssl7) In Complex With Fc Of Human Iga1 | |
| Chain A, Crystal Structure Of Human Fcari Bound To | gi\|31615935 |
| Iga1-Fc | |
| Chain A, Model Of Human Iga1 Determined By | gi\|40890038 |
| Solution | |
| Scattering Curve-Fitting And Homology Modelling | |
| Chain A, Solution Structure Of Human Dimeric | gi\|166007134 |
| Immunoglobulin A | |
| Chain A, Solution Structure Of Human Secretory Iga1 | gi\|219109282 |
| Chain A, Solution Structure Of Human | gi\|219109294 |
| Siga2 | |
| Chain A, Solution Structure Of The Complex Between | gi\|88192781 |
| Immunoglobulin Iga1 And Human Serum Albumin | |
| Chain B, Crystal Structure Of A Staphylococcus | gi\|158430163 |
| Aureus | |
| Protein (Ssl7) In Complex With Fc Of Human Iga1 | |
| Chain B, Crystal Structure Of Human Fcari Bound To | gi\|31615936 |
| Iga1-Fc | |
| Chain B, Model Of Human Iga1 Determined By | gi\|40890039 |
| Solution | |
| Scattering Curve-Fitting And Homology Modelling | |
| Chain B, Model Of Human Iga2 Determined By | gi\|56553653 |
| Solution | |
| Scattering, Curve Fitting And Homology Modelling | |
| Chain B, Solution Structure Of Human Dimeric | gi\|166007135 |
| Immunoglobulin A | |
| Chain B, Solution Structure Of Human Secretory Iga1 | gi\|219109283 |
| Chain B, Solution Structure Of Human | gi\|219109295 |
| Siga2 | |
| Chain B, Solution Structure Of The Complex Between | gi\|88192782 |
| Immunoglobulin Iga1 And Human Serum Albumin | |
| Chain C, Solution Structure Of Human Dimeric | gi\|166007138 |
| Immunoglobulin A | |
| Chain C, Solution Structure Of Human Secretory Iga1 | gi\|219109285 |
| Chain D, Model Of Human Iga2 Determined By | gi\|56553655 |
| Solution | |
| Scattering, Curve Fitting And Homology Modelling | |
| Chain D, Solution Structure Of Human Dimeric | gi\|166007139 |
| Immunoglobulin A | |
| Chain D, Solution Structure Of Human Secretory Iga1 | gi\|219109284 |
| Chain D, Solution Structure Of Human | gi\|219109297 |
| Siga2 | |
| IGH@ protein [Homo sapiens] | gi\|41351320 |
| Ig A1 Bur | gi\|229585 |
| Ig alpha-2 chain-human (fragment) | gi\|2135473 |
| Immunoglobulin heavy chain variant [Homo sapiens] | gi\|9367869 |
| RecName: Full = Ig alpha-1 chain C | gi\|113584 |
| region | |
| RecName: Full = Ig alpha-2 chain C | gi\|218512088 |
| region | |
| SNC66 protein [Homo sapiens] | gi\|17224464 |
| SNC73 protein [Homo sapiens] | gi\|3201900 |
| hCG1988537 [Homo sapiens] | gi\|119570893 |
| hypothetical protein [Homo sapiens] | gi\|34365139 |
| hypothetical protein [Homo sapiens] | gi\|34365085 |
| hypothetical protein [Homo sapiens] | gi\|34364649 |
| hypothetical protein [Homo sapiens] | gi\|34364645 |
| hypothetical protein [Homo sapiens] | gi\|31874190 |
| hypothetical protein [Homo sapiens] | gi\|31874206 |
| hypothetical protein [Homo sapiens] | gi\|34365128 |
| immunoglobulin A heavy chain allotype 2 [Homo | gi\|546799 |
| sapiens] | |
| immunoglobulin alpha 1 heavy chain constant region | gi\|50301689 |
| [Homo sapiens] | |
| immunoglobulin alpha 2m(1) heavy chain constant | gi\|50301691 |
| region [Homo sapiens] | |
| immunoglobulin alpha-1 heavy chain constant region | gi\|184749 |
| [Homo sapiens] | |
| immunoglobulin alpha-2 heavy chain [Homo sapiens] | gi\|184761 |
| immunoglobulin heavy chain constant alpha 1 | gi\|169249224 |
| membrane bound isoform 1 [Homo | |
| sapiens] | |
| immunoglobulin heavy chain constant alpha 1 | gi\|169249225 |
| membrane bound isoform 2 [Homo | |
| sapiens] | |
| immunoglobulin heavy chain constant region alpha 1 | gi\|12054070 |
| [Homo sapiens] | |
| unnamed protein product [Homo sapiens] | gi\|34536154 |
| unnamed protein product [Homo sapiens] | gi\|34536000 |
| unnamed protein product [Homo sapiens] | gi\|34535864 |
| unnamed protein product [Homo sapiens] | gi\|34527275 |
| unnamed protein product [Homo sapiens] | gi\|34527280 |
| unnamed protein product [Homo sapiens] | gi\|34527290 |
| unnamed protein product [Homo sapiens] | gi\|34527318 |
| unnamed protein product [Homo sapiens] | gi\|34527351 |
| unnamed protein product [Homo sapiens] | gi\|34527679 |
| unnamed protein product [Homo sapiens] | gi\|34535045 |
| unnamed protein product [Homo sapiens] | gi\|34533060 |
| unnamed protein product [Homo sapiens] | gi\|34532816 |
| unnamed protein product [Homo sapiens] | gi\|40038514 |
| unnamed protein product [Homo sapiens] | gi\|45502401 |
| unnamed protein product [Homo sapiens] | gi\|16554028 |
| unnamed protein product [Homo sapiens] | gi\|34526163 |
| unnamed protein product [Homo sapiens] | gi\|34526199 |
| unnamed protein | |
| product [Homo sapiens] | |
| unnamed protein product [Homo sapiens] | gi\|34527233 |
| unnamed protein product [Homo sapiens] | gi\|34527259 |
| unnamed protein product [Homo sapiens] | gi\|34532724 |
| unnamed protein product [Homo sapiens] | gi\|34531264 |
| unnamed protein product [Homo sapiens] | gi\|34531055 |
| unnamed protein product [Homo sapiens] | gi\|34529647 |
| unnamed protein product [Homo sapiens] | gi\|34529119 |
| unnamed protein product [Homo sapiens] | gi\|22760231 |
| unnamed protein product [Homo sapiens] | gi\|14042015 |

Peptide Information

| | | | | Start | | |
|---|---|---|---|---|---|---|
| Calc. Mass | Obsrv. Mass | ±da | ±ppm | Seq. | End Seq. | Sequence |
| 1835.9498 | 1835.9573 | .0075 | 4 | 392 | 408 | QEPSQGTTTFAV |
| | | | | | | TSILR |

| | |
|---|---|
| Ig Aalpha1 Bur | gi\|223099 |

Peptide Information

| Calc. | Obsrv. | | | Start | End | |
|---|---|---|---|---|---|---|
| Mass | Mass | ±da | ±ppm | Seq. | Seq. | Sequence |
| 1835.9498 | 1835.9573 | .0075 | 4 | 402 | 418 | QEPSZGTTTFA |
| | | | | | | VTSILR |

immunoglobulin heavy chain [Homo sapiens]
gi|46253743

Peptide Information

| | Obsrv. | | | Start | End | |
|---|---|---|---|---|---|---|
| Calc. Mass | Mass | ±da | ±ppm | Seq. | Seq. | Sequence |
| 928.437 | 928.4557 | .0187 | 20 | 49 | 56 | YSSSLESR |

| | |
|---|---|
| RecName: Full = Brefeldin | gi\|147742985 |
| A-inhibited guanine | |
| nucleotide-exchange protein 3 | |

Protein Group

| | |
|---|---|
| BIG3 [Homo sapiens] | gi\|15625588 |
| KIAA1244 [Homo sapiens] | gi\|223460210 |
| KIAA1244 [Homo sapiens] | gi\|168983997 |
| KIAA1244 [Homo sapiens] | gi\|119568303 |
| KIAA1244 [Homo sapiens] | gi\|168983855 |
| KIAA1244 [Homo sapiens] | gi\|168984026 |
| KIAA1244 protein [Homo sapiens] | gi\|40225579 |
| KIAA1244 protein [Homo sapiens] | gi\|20521814 |
| KIAA1244 protein [Homo sapiens] | gi\|21619911 |
| brefeldin A-inhibited guanine nucleotide-exchange | gi\|154350241 |
| protein 3 [Homo sapiens] | |
| hypothetical protein [Homo sapiens] | gi\|251733302 |
| unnamed protein product [Homo sapiens] | gi\|108250404 |

Peptide Information

| | Obsrv. | | | Start | End | |
|---|---|---|---|---|---|---|
| Calc. Mass | Mass | ±da | ±ppm | Seq. | Seq. | Sequence |
| 928.4444 | 928.4557 | .0113 | 12 | 2013 | 2020 | IYTMAADK |

| | |
|---|---|
| PHD finger protein 6 [Homo sapiens] | gi\|57209488 |

Protein Group

| | |
|---|---|
| KIAA1823 protein [Homo sapiens] | gi\|14017863 |
| PHD finger protein 6 [Homo sapiens] | gi\|57209486 |
| PHD finger protein 6 isoform 1 [Homo sapiens] | gi\|62865858 |
| PHD finger protein 6 isoform 1 [Homo sapiens] | gi\|28557677 |
| PHD finger protein 6, isoform CRA_b [Homo sapiens] | gi\|119632171 |
| PHD finger protein 6, isoform CRA_b [Homo sapiens] | gi\|119632168 |
| PHD finger protein 6, isoform CRA_d [Homo sapiens] | gi\|119632170 |
| PHD-like zinc finger protein [Homo sapiens] | gi\|27497549 |
| RecName: Full = PHD finger protein 6; AltName: | gi\|42559482 |
| Full = PHD-like zinc finger protein | |
| unnamed protein product [Homo sapiens] | |
| unnamed protein product [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 928.4193 | 928.4557 | .0364 | 39 | 314 | 320 | YIENMSR |

| | |
|---|---|
| zinc finger, CCHC domain containing | gi\|119582703 |
| 10, isoform CRA_d [Homo sapiens] | |

Protein Group

| | |
|---|---|
| RecName: Full = Zinc finger CCHC domain-containing | gi\|84028163 |
| protein 10 | |
| ZCCHC10 protein [Homo sapiens] | gi\|18490660 |
| Zinc finger, CCHC domain containing 10 [Homo | gi\|13528822 |
| sapiens] | |
| unnamed protein product [Homo sapiens] | gi\|194373877 |
| unnamed protein product [Homo sapiens] | gi\|193784674 |
| unnamed protein product [Homo sapiens] | gi\|7019971 |
| unnamed protein product [Homo sapiens] | gi\|40043232 |
| unnamed protein product [Homo sapiens] | gi\|40977976 |
| zinc finger CCHC domain-containing protein 10 [Homo | gi\|8923106 |
| sapiens] | |
| zinc finger, CCHC domain containing 10, isoform | gi\|119582700 |
| CRA_a [Homo sapiens] | |
| zinc finger, CCHC domain containing 10, isoform | gi\|119582701 |
| CRA_b [Homo sapiens] | |
| zinc finger, CCHC domain containing 10, isoform | gi\|119582702 |
| CRA_c [Homo sapiens] | |
| zinc finger, CCHC domain containing 10, isoform | gi\|119582704 |
| CRA_e [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 928.5111 | 928.4557 | −.0554 | −60 | 64 | 70 | YLHRPSR |

| | |
|---|---|
| class E basic helix-loop-helix protein | gi\|13540521 |
| 41 [Homo sapiens] | |

Protein Group

| | |
|---|---|
| Basic helix-loop-helix family, member e41 [Homo | gi\|19684064 |
| sapiens] | |
| RecName: Full = Class E basic helix-loop-helix protein | gi\|20137459 |
| 41; Short = bHLHe41; AltName: | |
| Full = Class B basic helix-loop-helix | |
| protein 3; Short = bHLHb3; AltName: | |
| Full = Differentially expressed in | |
| chondrocytes protein | |
| 2; | |
| Short = hDEC2; AltName: Full = Enhancer-of-split and | |
| hairy-related protein 1; Short = SHARP-1 | |
| bHLH protein DEC2 [Homo sapiens] | gi\|12583669 |
| basic helix-loop-helix domain containing, class B, 3, | gi\|119616934 |
| isoform CRA_a [Homo sapiens] | |
| basic helix-loop-helix domain containing, class B, 3, | gi\|119616933 |
| isoform CRA_a [Homo sapiens] | |
| basic helix-loop-helix domain-containing [Homo | gi\|121647001 |
| sapiens] | |
| basic helix-loop-helix protein 3 isoform 1 [Homo | gi\|55669468 |
| sapiens] | |
| unnamed protein product [Homo sapiens] | gi\|221044670 |

Peptide Information

| Calc. | Obsrv. | | | Start | End | |
|---|---|---|---|---|---|---|
| Mass | Mass | ±da | ±ppm | Seq. | Seq. | Sequence |
| 1340.6263 | 1340.6824 | .0561 | 42 | 1 | 11 | MDEGIPH |
| | | | | | | LQER |

| | |
|---|---|
| RecName: Full = Alkaline phosphatase, | gi\|68067533 |
| tissue-nonspecific isozyme; Short = | |
| AP-TNAP; Short = TNSALP; AltName: | |
| Full = Alkaline phosphatase | |
| liver/bone/kidney | |
| isozyme; Flags: | |
| Precursor | |

Protein Group

| | |
|---|---|
| Alkaline phosphatase, liver/bone/kidney [Homo sapiens] | gi\|187954299 |
| Alkaline phosphatase, liver/bone/kidney [Homo sapiens] | gi\|127801853 |
| Alkaline phosphatase, liver/bone/kidney [Homo sapiens] | gi\|127799935 |
| Alkaline phosphatase, liver/bone/kidney [Homo sapiens] | gi\|127799243 |
| Alkaline phosphatase, liver/bone/kidney [Homo sapiens] | gi\|118835602 |
| Alkaline phosphatase, liver/bone/kidney [Homo sapiens] | gi\|127796485 |
| alkalin phosphatase [Homo sapiens] | gi\|3401945 |
| alkaline phosphatase [Homo sapiens] | gi\|178462 |
| alkaline phosphatase, liver/bone/kidney [Homo sapiens] | gi\|55959694 |
| alkaline phosphatase, liver/bone/kidney [Homo sapiens] | gi\|55665927 |
| alkaline phosphatase, liver/bone/kidney, isoform | gi\|119615382 |
| CRA_a | |
| [Homo sapiens] | |
| alkaline phosphatase, liver/bone/kidney, isoform | gi\|119615381 |
| CRA_a | |
| [Homo sapiens] | |
| alkaline phosphatase, liver/bone/kidney, isoform | gi\|119615380 |
| CRA_a | |
| [Homo sapiens] | |
| alkaline phosphatase, liver/bone/kidney, isoform | gi\|119615383 |
| CRA_b | |
| [Homo sapiens] | |
| alkaline phosphatase, tissue-nonspecific isozyme | gi\|116734717 |
| isoform 1 precursor [Homo sapiens] | |
| alkaline phosphatase, tissue-nonspecific isozyme | gi\|294660770 |
| isoform 2 [Homo sapiens] | |
| alkaline phosphatase, tissue-nonspecific isozyme | gi\|294660772 |
| isoform 3 [Homo sapiens] | |
| unnamed protein product [Homo sapiens] | gi\|221040844 |
| unnamed protein product [Homo sapiens] | gi\|28738 |
| unnamed protein product [Homo sapiens] | gi\|221042090 |
| unnamed protein product [Homo sapiens] | gi\|189069211 |
| unnamed protein product [Homo sapiens] | gi\|221039408 |

Peptide Information

| | | | | Start | End | |
|---|---|---|---|---|---|---|
| Calc. Mass | Obsrv. Mass | ±da | ±ppm | Seq. | Seq. | Sequence |
| 1835.9028 | 1835.9573 | .0545 | 30 | 137 | 152 | SRCNTTQGNEVT |
| | | | | | | SILR |

| | |
|---|---|
| eukaryotic translation initiation factor 3 | gi\|312433975 |
| subunit C [Homo sapiens] | |

Protein Group

| | |
|---|---|
| EIF3CL protein [Homo sapiens] | gi\|187956832 |
| Eukaryotic translation initiation factor 3, subunit C | gi\|187957270 |
| [Homo sapiens] | |
| Eukaryotic translation initiation factor 3, subunit C | gi\|16306765 |
| [Homo sapiens] | |
| Eukaryotic translation initiation factor 3, subunit C | gi\|47939099 |
| [Homo sapiens] | |
| RecName: Full = Eukaryotic translation initiation | gi\|6685539 |
| factor 3 subunit C; Short = eIF3c; AltName: | |
| Full = Eukaryotic translation initiation factor 3 | |
| subunit 8; AltName: Full = eIF3 p110 | |
| Translation initiation factor eIF-p110 [Homo sapiens] | gi\|3337383 |
| cell migration-inducing protein 17 [Homo sapiens] | gi\|59803473 |
| eIF-3 p110 subunit [Homo sapiens] | gi\|1931584 |
| eukaryotic translation initiation factor 3 subunit C [Homo | gi\|153791492 |
| sapiens] | |
| eukaryotic translation initiation factor 3 subunit C [Homo | gi\|83700233 |
| sapiens] | |
| eukaryotic translation initiation factor 3 subunit C [Homo | gi\|4503525 |
| sapiens] | |
| eukaryotic translation initiation factor 3, subunit 8, | gi\|119570881 |
| 110 kDa, isoform CRA_a [Homo sapiens] | |
| eukaryotic translation initiation factor 3, subunit 8, | gi\|119570877 |
| 110 kDa, isoform CRA_a [Homo sapiens] | |
| eukaryotic translation initiation factor 3, subunit 8, | gi\|119570879 |
| 110 kDa, isoform CRA_a [Homo sapiens] | |
| eukaryotic translation initiation factor 3, subunit 8, | gi\|119570880 |
| 110 kDa, isoform CRA_b [Homo sapiens] | |
| eukaryotic translation initiation factor 3, subunit 8, | gi\|119570878 |
| 110 kDa, isoform CRA_b [Homo sapiens] | |
| translation intiation factor eIF-3 p110 subunit [Homo | gi\|1718197 |
| sapiens] | |
| unnamed protein product [Homo sapiens] | gi\|194385866 |
| unnamed protein product [Homo sapiens] | gi\|194383552 |
| unnamed protein product [Homo sapiens] | gi\|194376092 |
| unnamed protein product [Homo sapiens] | gi\|194375309 |
| unnamed protein product [Homo sapiens] | gi\|194373399 |
| unnamed protein product [Homo sapiens] | gi\|7021015 |
| unnamed protein product [Homo sapiens] | gi\|158257742 |
| unnamed protein product [Homo sapiens] | gi\|194387978 |
| unnamed protein product [Homo | gi\|193786223 |
| sapiens] | |

Peptide Information

| Calc. | | | | Start | End | |
|---|---|---|---|---|---|---|
| Mass | Obsrv. Mass | ±da | ±ppm | Seq. | Seq. | Sequence |
| 928.4886 | 928.4557 | −.0329 | −35 | 554 | 560 | YIYAKDR |

| | |
|---|---|
| BarH-like homeobox 1 [Homo sapiens] | gi\|219521562 |

Peptide Information

| | Obsrv. | | | Start | End | |
|---|---|---|---|---|---|---|
| Calc. Mass | Mass | ±da | ±ppm | Seq. | Seq. | Sequence |
| 928.4886 | 928.4557 | −.0329 | −35 | 202 | 208 | YLSVQYR |

| | |
|---|---|
| eukaryotic translation initiation | gi\|119598673 |
| factor 4 gamma, 1, isoform | |
| CRA_g [Homo sapiens] | |

Protein Group

| | |
|---|---|
| EIF4G1 protein [Homo sapiens] | gi\|187956781 |
| EIF4G1 protein [Homo sapiens] | gi\|187954575 |
| EIF4G1 protein [Homo sapiens] | gi\|14043628 |
| EIF4G1 variant protein [Homo sapiens] | gi\|68533081 |
| RecName: Full = Eukaryotic translation initiation | gi\|294862538 |
| factor 4 gamma 1; Short = eIF-4-gamma 1; | |
| Short = eIF-4G 1; Short = eIF-4G1; | |
| AltName: Full = p220 | |
| eukaryotic protein synthesis initiation factor [Homo | gi\|3941724 |
| sapiens] | |
| eukaryotic translation initiation factor 4 gamma 1 | gi\|302699237 |
| isoform 1 [Homo sapiens] | |
| eukaryotic translation initiation factor 4 gamma 1 | gi\|38201627 |
| isoform 2 [Homo sapiens] | |
| eukaryotic translation initiation factor 4 gamma 1 | gi\|38201625 |
| isoform 3 [Homo sapiens] | |
| eukaryotic translation initiation factor 4 gamma 1 | gi\|302699239 |
| isoform 4 [Homo sapiens] | |
| eukaryotic translation initiation factor 4 gamma 1 | gi\|38201623 |
| isoform 5 [Homo sapiens] | |
| eukaryotic translation initiation factor 4 gamma 1 | gi\|302699247 |
| isoform 6 [Homo sapiens] | |
| eukaryotic translation initiation factor 4 gamma 1 | gi\|302699245 |
| isoform 6 [Homo sapiens] | |
| eukaryotic translation initiation factor 4 gamma, 1 | gi\|30583403 |
| [Homo sapiens] | |
| eukaryotic translation initiation factor 4 gamma, 1, | gi\|119598663 |
| isoform CRA_a [Homo sapiens] | |
| eukaryotic translation initiation factor 4 gamma, 1, | gi\|119598672 |
| isoform CRA_c [Homo sapiens] | |
| eukaryotic translation initiation factor 4 gamma, 1, | gi\|119598669 |
| isoform CRA_c [Homo sapiens] | |
| eukaryotic translation initiation factor 4 gamma, 1, | gi\|119598665 |
| isoform CRA_c [Homo sapiens] | |
| eukaryotic translation initiation factor 4 gamma, 1, | gi\|119598666 |
| isoform CRA_d [Homo sapiens] | |
| eukaryotic translation initiation factor 4 gamma, 1, | gi\|119598667 |
| isoform CRA_e [Homo sapiens] | |
| eukaryotic translation initiation factor 4 gamma, 1, | gi\|119598671 |
| isoform CRA_f [Homo sapiens] | |
| eukaryotic translation initiation factor 4 gamma, 1, | gi\|119598668 |
| isoform CRA_f [Homo sapiens] | |
| eukaryotic translation initiation factor 4 gamma, 1, | gi\|119598670 |
| isoform CRA_g [Homo sapiens] | |
| eukaryotic translation initiation factor 4 gamma, 1, | gi\|119598674 |
| isoform CRA_h [Homo sapiens] | |
| eukaryotic translation initiation factor 4G I [Homo | gi\|21634449 |
| sapiens] | |
| eukaryotic translation initiation factor 4GI [Homo | gi\|21655146 |
| sapiens] | |
| hypothetical protein [Homo sapiens] | gi\|57997536 |
| unnamed protein product [Homo sapiens] | gi\|194389380 |
| unnamed protein product [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 1340.7355 | 1340.6824 | −.0531 | −40 | 929 | 939 | EHIKVQ |
| | | | | | | QLMAK |

| | |
|---|---|
| exocyst complex component 6, | gi\|119570470 |
| isoform CRA_d [Homo sapiens] | |

Protein Group

| | |
|---|---|
| Exocyst complex component 6 [Homo sapiens] | gi\|34783239 |
| RecName: Full = Exocyst complex component 6; | gi\|212287926 |
| AltName: Full = Exocyst complex component | |
| Sec15A; AltName: Full = SEC15-like protein 1 | |
| SEC15-like protein 3 [Homo sapiens] | gi\|148608033 |
| exocyst complex component 6 [Homo sapiens] | gi\|123231220 |
| exocyst complex component 6 [Homo sapiens] | gi\|55665929 |
| exocyst complex component 6 [Homo sapiens] | gi\|123227366 |
| exocyst complex component 6 isoform a [Homo | gi\|65507403 |
| sapiens] | |
| exocyst complex component 6 isoform b [Homo | gi\|62243652 |
| sapiens] | |
| exocyst complex component 6, isoform CRA_a [Homo | gi\|119570467 |
| sapiens] | |
| exocyst complex component 6, isoform CRA_b [Homo | gi\|119570468 |
| sapiens] | |
| exocyst complex component 6, isoform CRA_c [Homo | gi\|119570469 |
| sapiens] | |
| exocyst complex component 6, isoform CRA_e [Homo | gi\|119570471 |
| sapiens] | |
| hypothetical protein [Homo sapiens] | gi\|6807998 |
| unnamed protein product [Homo sapiens] | gi\|221043148 |
| unnamed protein product [Homo sapiens] | gi\|194375816 |
| unnamed protein product [Homo sapiens] | gi\|40032933 |
| unnamed protein product [Homo sapiens] | gi\|108250166 |
| unnamed protein product [Homo sapiens] | gi\|193785860 |
| unnamed protein product [Homo sapiens] | gi\|189054938 |
| unnamed protein product [Homo sapiens] | gi\|7023795 |
| unnamed protein product | |
| [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 928.5138 | 928.4557 | −.0581 | −63 | 583 | 590 | LYGLSTFK |

| | |
|---|---|
| RecName: Full = Protein FAM183B | gi\|215275260 |

Protein Group

| | |
|---|---|
| hCG1645872 [Homo sapiens] | gi\|119614518 |
| hypothetical gene supported by BC045803 [Homo | gi\|51094744 |
| sapiens] | |
| testis highly expressed protein 6 [Homo sapiens] | gi\|225734648 |
| unnamed protein product [Homo sapiens] | gi\|261385756 |
| unnamed protein product [Homo sapiens] | gi\|40046854 |

Peptide Information

| | Obsrv. | | | Start | End | |
|---|---|---|---|---|---|---|
| Calc. Mass | Mass | ±da | ±ppm | Seq. | Seq. | Sequence |
| 1835.9723 | 1835.9573 | −.015 | −8 | 7 | 21 | ERVVTDEVHQNQILR |

| | |
|---|---|
| SWI/SNF related, matrix associated, | gi\|119579218 |
| actin dependent regulator of chromatin, | |
| subfamily a, member 2, isoform | |
| CRA_a [Homo sapiens] | |

Protein Group

| | |
|---|---|
| HBRM [Homo sapiens] | gi\|414117 |
| RecName: Full = Probable global transcription activator | gi\|212276472 |
| SNF2L2; AltName: Full = ATP-dependent helicase | |
| SMARCA2; AltName: Full = BRG1-associated factor | |
| 190B; Short = BAF190B; AltName: Full = Protein | |
| brahma homolog; Short = hBRM; AltName: | |
| Full = SNF2-alpha; AltName: Full = SWI/SNF- | |
| related matrix-associated actin-dependent | |
| regulator of chromatin subfamily A | |
| member 2 | |
| SWI/SNF related, matrix associated, actin dependent | gi\|55957478 |
| regulator of chromatin, subfamily a, member 2 [Homo | |
| sapiens] | |
| SWI/SNF related, matrix associated, actin dependent | gi\|55958984 |
| regulator of chromatin, subfamily a, member 2 [Homo | |
| sapiens] | |
| SWI/SNF related, matrix associated, actin dependent | gi\|55958983 |
| regulator of chromatin, subfamily a, member 2 [Homo | |
| sapiens] | |
| SWI/SNF related, matrix associated, actin dependent | gi\|55957477 |
| regulator of chromatin, subfamily a, member 2 [Homo | |
| sapiens] | |
| SWI/SNF related, matrix associated, actin dependent | gi\|55958982 |
| regulator of chromatin, subfamily a, | |
| member 2 [Homo sapiens] | |
| SWI/SNF related, matrix associated, actin | gi\|119579215 |
| dependent | |
| regulator of chromatin, subfamily a, | |
| member 2, isoform | |
| CRA_a [Homo sapiens] | |
| SWI/SNF related, matrix associated, actin | gi\|119579217 |
| dependent | |
| regulator of chromatin, subfamily a, | |
| member 2, isoform | |
| CRA_c [Homo sapiens] | |
| SWI/SNF related, matrix associated, actin | gi\|119579219 |
| dependent | |
| regulator of chromatin, subfamily a, | |
| member 2, isoform | |
| CRA_c [Homo sapiens] | |
| probable global transcription activator | gi\|48255900 |
| SNF2L2 isoform | |
| a [Homo sapiens] | |
| probable global transcription activator | gi\|48255898 |
| SNF2L2 isoform | |
| b [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 1405.7039 | 1405.7631 | .0592 | 42 | 391 | 401 | QLRQEV |
| | | | | | | VACMR |

| | |
|---|---|
| C protein [Homo sapiens] | gi\|36501 |

Peptide Information

| | Obsrv. | | | Start | End | |
|---|---|---|---|---|---|---|
| Calc. Mass | Mass | ±da | ±ppm | Seq. | Seq. | Sequence |
| 949.4924 | 949.515 | .0226 | 24 | 972 | 978 | KSMEWLR |

| | |
|---|---|
| transducin-like enhancer | gi\|55959926 |
| of split 4 (E(sp1) homolog, | |
| Drosophila) [Homo sapiens] | |

Protein Group

| | |
|---|---|
| transducin-like enhancer of split 4 (E(sp1) | gi\|55958795 |
| homolog, | |
| Drosophila) [Homo sapiens] | |
| transducin-like enhancer of split 4 (E(sp1) | gi\|57208918 |
| homolog, | |
| Drosophila) [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 850.4417 | 850.405 | −.0367 | −43 | 198 | 205 | VSPSASFR |

| | |
|---|---|
| leucine rich repeat containing 27, | gi\|119569507 |
| isoform CRA_b [Homo sapiens] | |

Protein Group

| | |
|---|---|
| KIAA1674 protein [Homo sapiens] | gi\|12697893 |
| RecName: Full = Leucine-rich repeat-containing protein | gi\|85701139 |
| 27 | |
| leucine rich repeat containing 27, isoform CRA_a | gi\|119569506 |
| [Homo sapiens] | |
| leucine rich repeat containing 27, isoform CRA_a | gi\|119569508 |
| [Homo sapiens] | |
| leucine-rich repeat-containing protein 27 isoform a | gi\|219555700 |
| [Homo sapiens] | |
| leucine-rich repeat-containing protein 27 isoform a | gi\|55741567 |
| [Homo sapiens] | |
| novel protein [Homo sapiens] | gi\|55960576 |
| novel protein [Homo sapiens] | gi\|55960574 |
| novel protein [Homo sapiens] | |
| unnamed protein product [Homo sapiens] | gi\|194376382 |
| unnamed protein product [Homo sapiens] | gi\|193783556 |
| unnamed protein product [Homo sapiens] | gi\|21752065 |

Peptide Information

| Calc. | Obsrv. | | | Start | End | |
|---|---|---|---|---|---|---|
| Mass | Mass | ±da | ±ppm | Seq. | Seq. | Sequence |
| 1340.6852 | 1340.6824 | −.0028 | −2 | 381 | 390 | QHVLQ |
| | | | | | | MREQR |

### 3—Prothrombin Complex Concentrate

Below is the sequence of the 8 new found proteins which will be named KH11, KH12, KH13, KH14, KH15, KH16, KH17 and KH18 and its functions are under investigation.

| | | | |
|---|---|---|---|
| | 60/C9 | | Instr./Gel |
| | | | Origin |
| | [1] Sample Project | | Instrument |
| | 20110503 | | Sample |
| | | | Name |
| Protein Name - | KH11 (SEQ ID NO. KH11) | Accession No. | |

| | |
|---|---|
| unnamed protein product [Homo sapiens] | gi\|215415638 |

Peptide Information

| Calc. | Obsrv. | | | Start | End | |
|---|---|---|---|---|---|---|
| Mass | Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1311.5555 | 1311.5947 | .0392 | 30 | 78 | 88 | ETEGLCQEMSK |
| 1318.642 | 1318.6689 | .0269 | 20 | 141 | 151 | LSPLGEEMRDR |
| 1327.5505 | 1327.6265 | .076 | 57 | 78 | 88 | ETEGLCQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDS |
| | | | | | | GR |
| 1815.8508 | 1815.8446 | −.0062 | −3 | 24 | 40 | DSGRDYVSQFEGS |
| | | | | | | ALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEFWDNLEK |

| | |
|---|---|
| Chain A, Solution Structure Of Double Super | gi\|293651900 |
| Helix Model | |

Protein Group

| | |
|---|---|
| Chain A, Crystal Structure Of Lipid-Free Human | gi\|90108664 |
| Apolipoprotein A-I | |
| Chain B, Crystal Structure Of Lipid-Free Human | gi\|90108665 |
| Apolipoprotein A-I | |
| Chain B, Solution Structure Of Double Super Helix | gi\|293651901 |
| Model | |
| Chain C, Crystal Structure Of Lipid-Free Human | gi\|90108666 |
| Apolipoprotein A-I | |
| apoA1 [Homo sapiens] | gi\|490098 |
| apoAI [Homo sapiens] | gi\|1330306 |
| unnamed protein product [Homo sapiens] | gi\|215415678 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQ |
| | | | | | | KK |
| 1386.7152 | 1386.7365 | .0213 | 15 | 227 | 238 | VSFLSALEE |
| | | | | | | YTK |
| 1400.6693 | 1400.715 | .0457 | 33 | 28 | 40 | DYVSQFEGS |
| | | | | | | ALGK |
| 1411.6675 | 1411.6667 | −.0008 | −1 | 107 | 116 | KWQEEMEL |
| | | | | | | YR |
| 1453.707 | 1453.7174 | .0104 | 7 | 1 | 12 | DEPPQSPW |
| | | | | | | DRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLEK |

| | |
|---|---|
| unnamed protein product [Homo sapiens] | gi\|215415636 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| | 1585.8174 | | | | | |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |

| | |
|---|---|
| unnamed protein product [Homo sapiens] | gi\|215415652 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.6273 | 1283.6271 | −.0002 | 0 | 150 | 160 | DCARAHVDALR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1311.5555 | 1311.5947 | .0392 | 30 | 78 | 88 | ETEGLCQEMSK |
| 1327.5505 | 1327.6265 | .076 | 57 | 78 | 88 | ETEGLCQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDS |
| | | | | | | GR |
| 1815.8508 | 1815.8446 | −.0062 | −3 | 24 | 40 | DSGRDYVSQFEGS |
| | | | | | | ALGK |
| 1815.8508 | 1815.8446 | −.0062 | −3 | 24 | 40 | DSGRDYVSQFEGS |
| | | | | | | ALGK |
| 2202.1189 | 2202.0879 | −.031 | −14 | 60 | 77 | LREQLGPVTQEFW |
| | | | | | | DNLEK |

| | |
|---|---|
| unnamed protein product [Homo sapiens] | gi\|215415640 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 4 | 18 | 19 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.6273 | 1283.6271 | −.0002 | 0 | 150 | 160 | DCARAHVDALR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLEK |

| | |
|---|---|
| unnamed protein product [Homo sapiens] | gi\|215415648 |

Peptide Information

| | Obsrv. | | | Start | End | |
|---|---|---|---|---|---|---|
| Calc. Mass | Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.6273 | 1283.6271 | −.0002 | 0 | 150 | 160 | DCARAHVDALR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |

| | |
|---|---|
| unnamed protein product [Homo sapiens] | gi\|215415654 |

Peptide Information

| | Obsrv. | | | Start | End | |
|---|---|---|---|---|---|---|
| Calc. Mass | Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| | 1283.6271 | | | | | |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1311.5555 | 1311.5947 | .0392 | 30 | 78 | 88 | ETEGLCQEMSK |
| 1318.642 | 1318.6689 | .0269 | 20 | 141 | 151 | LSPLGEEMRDR |
| 1327.5505 | 1327.6265 | .076 | 57 | 78 | 88 | ETEGLCQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDSGR |
| 1815.8508 | 1815.8446 | −.0062 | −3 | 24 | 40 | DSGRDYVSQFEGSALGK |
| 1815.8508 | 1815.8446 | −.0062 | −3 | 24 | 40 | DSGRDYVSQFEGSALGK |
| 2202.1189 | 2202.0879 | −.031 | −14 | 60 | 77 | LREQLGPVTQEFWDNLEK |

| | |
|---|---|
| unnamed protein product [Homo sapiens] | gi\|215415628 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLE K |

| | |
|---|---|
| unnamed protein product [Homo sapiens] | gi\|215415634 |

Peptide Information

| | Obsrv. | | | Start | End | |
|---|---|---|---|---|---|---|
| Calc. Mass | Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| | 1467.7849 | | | | | |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLEK |

| | |
|---|---|
| unnamed protein product [Homo sapiens] | gi\|215415650V |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1311.5555 | 1311.5947 | .0392 | 30 | 78 | 88 | ETEGLCQEMSK |
| 1318.642 | 1318.6689 | .0269 | 20 | 141 | 151 | LSPLGEEMRDR |
| 1327.5505 | 1327.6265 | .076 | 57 | 78 | 88 | ETEGLCQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWD |
| | | | | | | RVK |
| 1467.7914 | 1467.7849 | −.0065 | −4 | 119 | 131 | VEPLRAELQE |
| | | | | | | GAR |
| 1467.7914 | 1467.7849 | −.0065 | −4 | 119 | 131 | VEPLRAELQE |
| | | | | | | GAR |
| 1612.7854 | 1612.8048 | .0194 | 12 | 46 | 59 | LLDNWDSVTS |
| | | | | | | TFSK |
| 1650.8698 | 1650.8733 | .0035 | 2 | 13 | 27 | DLATVYVDVL |
| | | | | | | KDSGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLEK |

| | |
|---|---|
| unnamed protein product [Homo sapiens] | gi\|215415626 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDS |
| | | | | | | GR |
| 1815.8508 | 1815.8446 | −.0062 | −3 | 24 | 40 | DSGRDYVSQFEGS |
| | | | | | | ALGK |
| 1815.8508 | 1815.8446 | −.0062 | −3 | 24 | 40 | DSGRDYVSQFEGS |
| | | | | | | ALGK |
| 2202.1189 | 2202.0879 | −.031 | −14 | 60 | 77 | LREQLGPVTQEFW |
| | | | | | | DNLEK |

| | |
|---|---|
| proapolipoprotein [Homo sapiens] | gi\|178775 |

Peptide Information

| | Obsrv. | | | Start | End | |
|---|---|---|---|---|---|---|
| Calc. Mass | Mass | ±da | ±ppm | Seq. | Seq. | Sequence |
| 831.4359 | 831.4695 | .0336 | 40 | 195 | 201 | LAEYHAK |
| 869.5203 | 869.5491 | .0288 | 33 | 123 | 129 | QKVEPLR |
| 873.4424 | 873.4709 | .0285 | 33 | 130 | 137 | AELQEGAR |
| 1008.5697 | 1008.5928 | .0231 | 23 | 158 | 166 | ARAHVDALR |
| 1012.5786 | 1012.6014 | .0228 | 23 | 213 | 221 | AKPALEDLR |
| 1012.5786 | 1012.6014 | .0228 | 23 | 213 | 221 | AKPALEDLR |
| 1252.6208 | 1252.6487 | .0279 | 22 | 103 | 112 | VQPYLDDFQK |
| 1283.5725 | 1283.6271 | .0546 | 43 | 114 | 122 | WQEEMELYR |
| 1301.6484 | 1301.66 | .0116 | 9 | 167 | 177 | THLAPYSDELR |
| 1301.6484 | 1301.66 | .0116 | 9 | 167 | 177 | THLAPYSDELR |
| 1318.642 | 1318.6689 | .0269 | 20 | 147 | 157 | LSPLGEEMRDR |
| 1323.621 | 1323.6156 | −.0054 | −4 | 84 | 94 | ETEGLRQEMSK |
| 1380.7158 | 1380.7303 | .0145 | 11 | 103 | 113 | VQPYLDDFQKK |
| 1386.7152 | 1386.7365 | .0213 | 15 | 233 | 244 | VSFLSALEEYTK |
| 1400.6693 | 1400.715 | .0457 | 33 | 34 | 46 | DYVSQFEGSAL |
| | | | | | | GK |
| 1411.6675 | 1411.6667 | −.0008 | −1 | 113 | 122 | KWQEEMELYR |
| 1467.7914 | 1467.7849 | −.0065 | −4 | 125 | 137 | VEPLRAELQEGAR |
| 1467.7914 | 1467.7849 | −.0065 | −4 | 125 | 137 | VEPLRAELQEGAR |
| 1585.8081 | 1585.8174 | .0093 | 6 | 167 | 179 | THLAPYSDELRQR |
| 1585.8081 | 1585.8174 | .0093 | 6 | 167 | 179 | THLAPYSDELRQR |
| 1612.7854 | 1612.8048 | .0194 | 12 | 52 | 65 | LLDNWDSVTSTF |
| | | | | | | SK |
| 1650.8698 | 1650.8733 | .0035 | 2 | 19 | 33 | DLATVYVDVLKD |
| | | | | | | SGR |
| 1815.8508 | 1815.8446 | −.0062 | −3 | 30 | 46 | DSGRDYVSQFE |
| | | | | | | GSALGK |
| 1815.8508 | 1815.8446 | −.0062 | −3 | 30 | 46 | DSGRDYVSQFE |
| | | | | | | GSALGK |
| 2202.1189 | 2202.0879 | −.031 | −14 | 66 | 83 | LREQLGPVTQEF |
| | | | | | | WDNLEK |

| | |
|---|---|
| unnamed protein product [Homo sapiens] | gi\|215415624 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSP |
| | | | | | | WDR |
| 1226.5437 | 1226.5634 | .0197 | 16 | 1 | 10 | DEPPQSP |
| | | | | | | WDR |
| 1252.6208 | 1252.6487 | .0279 | 22 | 97 | 106 | VQPYLDD |
| | | | | | | FQK |
| 1283.5725 | 1283.6271 | .0546 | 43 | 108 | 116 | WQEEMEL |
| | | | | | | YR |
| 1301.6484 | 1301.66 | .0116 | 9 | 161 | 171 | THLAPYSD |
| | | | | | | ELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLE K |

| | |
|---|---|
| unnamed protein product [Homo sapiens] | gi\|215415646 |

Peptide Information

| | Obsrv. | | | Start | End | |
|---|---|---|---|---|---|---|
| Calc. Mass | Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| | 1323.6156 | | | | | |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDSGR |
| 1815.8508 | 1815.844 | −.0062 | −3 | 2 | 40 | DSGRDYVSQFE |
| 1815.8508 | 1815.844 | −.0062 | −3 | 2 | 40 | DSGRDYVSQFE |

| | |
|---|---|
| apolipoprotein A-I preproprotein [Homo sapiens] | gi\|4557321 |

Protein Group

| | |
|---|---|
| ApoA-I [Homo sapiens] | gi\|296729 |
| Apolipoprotein A-I [Homo sapiens] | gi\|82571472 |
| Apolipoprotein A-I [Homo sapiens] | gi\|13529242 |
| RecName: Full = Apolipoprotein A-I; Short = Apo-AI; | gi\|113992 |
| Short = ApoA-I; AltName: Full = Apolipoprotein A1; | |
| Contains: RecName: Full = Apolipoprotein | |
| A-I(1-242); Flags: Precursor | |
| apolipoprotein A-I [Homo sapiens] | gi\|37499465 |
| apolipoprotein A-I [Homo sapiens] | gi\|45593391 |
| apolipoprotein A-I precursor variant 1 [Homo sapiens] | gi\|167887482 |
| apolipoprotein A-I precursor variant 2 [Homo sapiens] | gi\|167887483 |
| apolipoprotein A-I precursor variant 3 [Homo sapiens] | gi\|167887484 |
| apolipoprotein A-I precursor variant 4 [Homo sapiens] | gi\|167887485 |
| apolipoprotein A-I precursor variant 5 [Homo sapiens] | gi\|167887486 |
| apolipoprotein A-I, isoform CRA_a [Homo sapiens] | gi\|119587680 |
| apolipoprotein A-I, isoform CRA_a [Homo sapiens] | gi\|119587679 |
| apolipoprotein A-I, isoform CRA_a [Homo sapiens] | gi\|119587678 |
| apolipoprotein AI precursor [Homo sapiens] | gi\|296635 |
| apolipoprotein AI precursor [Homo sapiens] | |
| preproapolipoprotein A-I [Homo sapiens] | gi\|178768 |
| preproapolipoprotein AI [Homo sapiens] | gi\|28772 |
| preproapolipoprotein [Homo sapiens] | gi\|512410 |
| proapolipoprotein [Homo sapiens] | gi\|178773 |
| unnamed protein product [Homo sapiens] | gi\|732753 |
| unnamed protein product [Homo sapiens] | gi\|158257894 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 213 | 219 | LAEYHAK |
| 869.5203 | 869.5491 | .0288 | 33 | 141 | 147 | QKVEPLR |
| 873.4424 | 873.4709 | .0285 | 33 | 148 | 155 | AELQEGAR |
| 1008.5697 | 1008.5928 | .0231 | 23 | 176 | 184 | ARAHVDALR |
| 1012.5786 | 1012.6014 | .0228 | 23 | 231 | 239 | AKPALEDLR |
| 1012.5786 | 1012.6014 | .0228 | 23 | 231 | 239 | AKPALEDLR |
| 1252.6208 | 1252.6487 | .0279 | 22 | 121 | 130 | VQPYLDDFQK |
| 1283.5725 | 1283.6271 | .0546 | 43 | 132 | 140 | WQEEMELYR |
| 1301.6484 | 1301.66 | .0116 | 9 | 185 | 195 | THLAPYSDELR |
| 1301.6484 | 1301.66 | .0116 | 9 | 185 | 195 | THLAPYSDELR |
| 1318.642 | 1318.6689 | .0269 | 20 | 165 | 175 | LSPLGEEMRDR |
| 1323.621 | 1323.6156 | −.0054 | −4 | 102 | 112 | ETEGLRQEMSK |
| 1380.7158 | 1380.7303 | .0145 | 11 | 121 | 131 | VQPYLDDFQKK |
| 1386.7152 | 1386.7365 | .0213 | 15 | 251 | 262 | VSFLSALEEYTK |
| 1400.6693 | 1400.715 | .0457 | 33 | 52 | 64 | DYVSQFEGSAL |
| | | | | | | GK |
| 1411.6675 | 1411.6667 | −.0008 | −1 | 131 | 140 | KWQEEMELYR |
| 1467.7914 | 1467.7849 | −.0065 | −4 | 143 | 155 | VEPLRAELQEGAR |
| 1467.7914 | 1467.7849 | −.0065 | −4 | 143 | 155 | VEPLRAELQEGAR |
| 1585.8081 | 1585.8174 | .0093 | 6 | 185 | 197 | THLAPYSDELRQR |
| 1585.8081 | 1585.8174 | .0093 | 6 | 185 | 197 | THLAPYSDELRQR |
| 1612.7854 | 1612.8048 | .0194 | 12 | 70 | 83 | LLDNWDSVTSTF |
| | | | | | | SK |
| 1650.8698 | 1650.8733 | .0035 | 2 | 37 | 51 | DLATVYVDVLKD |
| | | | | | | SGR |
| 1815.8508 | 1815.8446 | −.0062 | −3 | 48 | 64 | DSGRDYVSQFE |
| | | | | | | GSALGK |
| 1815.8508 | 1815.8446 | −.0062 | −3 | 48 | 64 | DSGRDYVSQFE |
| | | | | | | GSALGK |
| 2202.1189 | 2202.0879 | −.031 | −14 | 84 | 101 | LREQLGPVTQEF |
| | | | | | | WDNLEK |

| | |
|---|---|
| unnamed protein product [Homo sapiens] | gi\|15415644 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDS |
| | | | | | | GR |
| 1815.8508 | 1815.8446 | −.0062 | −3 | 24 | 40 | DSGRDYVSQFEGS |
| | | | | | | ALGK |
| 1815.8508 | 1815.8446 | −.0062 | −3 | 24 | 40 | DSGRDYVSQFEGS |
| | | | | | | ALGK |

| | |
|---|---|
| unnamed protein product [Homo | gi\|215415658 |
| sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| | 1226.5634 | | | | | |
| 1252.6208 | 1252.6487 | .0279 | 22 | 97 | 106 | VQPYLDDFQK |
| 1283.6273 | 1283.6271 | −.0002 | 0 | 150 | 160 | DCARAHVDALR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDS |
| | | | | | | GR |
| 1815.8508 | 1815.8446 | −.0062 | −3 | 24 | 40 | DSGRDYVSQFEGS |
| | | | | | | ALGK |
| 1815.8508 | 1815.8446 | −.0062 | −3 | 24 | 40 | DSGRDYVSQFEGS |
| | | | | | | ALGK |
| 2202.1189 | 2202.0879 | −.031 | −14 | 60 | 77 | LREQLGPVTQEFW |
| | | | | | | DNLEK |

| | |
|---|---|
| unnamed protein product [Homo sapiens] | gi\|215415656 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSP |
| | | | | | | WDR |
| 1252.6208 | 1252.6487 | .0279 | 22 | 97 | 106 | VQPYLDDF |
| | | | | | | QK |
| 1283.6273 | 1283.6271 | −.0002 | 0 | 150 | 160 | DCARAHV |
| | | | | | | DALR |
| 1301.6484 | 1301.66 | .0116 | 9 | 161 | 171 | THLAPYSD |
| | | | | | | ELR |
| 1301.6484 | 1301.66 | .0116 | 9 | 161 | 171 | THLAPYSD |
| | | | | | | ELR |
| 1323.621 | 1323.6156 | −.0054 | −4 | 78 | 88 | ETEGLRQE |
| | | | | | | MSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLEK |

| | |
|---|---|
| proapo-A-I protein [Homo sapiens] | gi\|178777 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 213 | 219 | LAEYHAK |
| 873.4424 | 873.4709 | .0285 | 33 | 148 | 155 | AELQEGAR |
| 1008.5697 | 1008.5928 | .0231 | 23 | 176 | 184 | ARAHVDALR |
| 1012.5786 | 1012.6014 | .0228 | 23 | 231 | 239 | AKPALEDLR |
| 1012.5786 | 1012.6014 | .0228 | 23 | 231 | 239 | AKPALEDLR |
| 1252.6208 | 1252.6487 | .0279 | 22 | 121 | 130 | VQPYLDDFQK |
| 1283.5725 | 1283.6271 | .0546 | 43 | 132 | 140 | WQEEMELYR |
| 1301.6484 | 1301.66 | .0116 | 9 | 185 | 195 | THLAPYSDELR |
| 1301.6484 | 1301.66 | .0116 | 9 | 185 | 195 | THLAPYSDELR |
| 1318.642 | 1318.6689 | .0269 | 20 | 165 | 175 | LSPLGEEMRDR |
| 1323.621 | 1323.6156 | −.0054 | −4 | 102 | 112 | ETEGLRQEMSK |
| 1380.7158 | 1380.7303 | .0145 | 11 | 121 | 131 | VQPYLDDFQKK |
| 1386.7152 | 1386.7365 | .0213 | 15 | 251 | 262 | VSFLSALEEYTK |
| 1400.6693 | 1400.715 | .0457 | 33 | 52 | 64 | DYVSQFEGSALGK |
| 1411.6675 | 1411.6667 | −.0008 | −1 | 131 | 140 | KWQEEMELYR |
| 1453.7758 | 1453.7174 | −.0584 | −40 | 143 | 155 | VDPLRAELQEGAR |
| 1585.8081 | 1585.8174 | .0093 | 6 | 185 | 197 | THLAPYSDELRQR |
| 1585.8081 | 1585.8174 | .0093 | 6 | 185 | 197 | THLAPYSDELRQR |
| 1612.7854 | 1612.8048 | .0194 | 12 | 70 | 83 | LLDNWDSVTSTFSK |
| 1650.8698 | 1650.8733 | .0035 | 2 | 37 | 51 | DLATVYVDVLKDS |
| | | | | | | GR |
| | 1650.8733 | | | | | |
| 1815.8508 | 1815.8446 | −.0062 | −3 | 48 | 64 | DSGRDYVSQFE |
| | | | | | | GSALGK |
| 1815.8508 | 1815.8446 | −.0062 | −3 | 48 | 64 | DSGRDYVSQFE |
| | | | | | | GSALGK |
| 2202.1189 | 2202.0879 | −.031 | −14 | 84 | 101 | LREQLGPVTQE |
| | | | | | | FWDNLE K |

| | |
|---|---|
| unnamed protein product [Homo sapiens] | gi\|215415642 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDS |
| | | | | | | GR |
| 1815.8508 | 1815.8446 | −.0062 | −3 | 24 | 40 | DSGRDYVSQFEGS |
| | | | | | | ALGK |
| 1815.8508 | 1815.8446 | −.0062 | −3 | 24 | 40 | DSGRDYVSQFEGS |
| | | | | | | ALGK |
| 2202.1189 | 2202.0879 | −.031 | −14 | 60 | 77 | LREQLGPVTQEFW |
| | | | | | | DNLE |

| | | | |
|---|---|---|---|
| | 63/C12 | | Instr./Gel Origin |
| | [1] Sample Project | | Instrument |
| | 20110503 | | Sample |
| | | | Name |
| Protein Name - | KH12 | Accession No. | |

| | |
|---|---|
| unnamed protein product [Homo | gi\|189066554 |
| sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 836.4009 | 836.415 | .0141 | 17 | 218 | 224 | GQQYQGR |
| 836.4009 | 836.415 | .0141 | 17 | 218 | 224 | GQQYQGR |
| 874.4781 | 874.4888 | .0107 | 12 | 501 | 508 | VTGWGNLK |
| 951.4894 | 951.5774 | .088 | 92 | 434 | 440 | YERNIEK |
| 971.425 | 971.4321 | .0071 | 7 | 87 | 94 | YTACETAR |
| 1148.5518 | 1148.5477 | −.0041 | −4 | 125 | 133 | SGIECQLWR |
| 1148.5518 | 1148.5477 | −.0041 | −4 | 125 | 133 | SGIECQLWR |
| 1189.579 | 1189.5732 | −.0058 | −5 | 600 | 608 | YGFYTHVFR |
| 1194.6001 | 1194.5807 | −.0194 | −16 | 354 | 363 | ELLESYIDGR |
| 1194.6001 | 1194.5807 | −.0194 | −16 | 354 | 363 | ELLESYIDGR |
| 1265.6022 | 1265.592 | −.0102 | −8 | 453 | 461 | YNWRENLDR |
| 1489.7223 | 1489.7014 | −.0209 | −14 | 597 | 608 | DGKYGFYTHVFR |
| 1542.62 | 1542.6259 | .0059 | 4 | 561 | 575 | GDACEGDSGGP |
| | | | | | | FVMK |
| 1561.7281 | 1561.6975 | −.0306 | −20 | 315 | 327 | TATSEYQTFFNPR |
| 1561.7281 | 1561.6975 | −.0306 | −20 | 315 | 327 | TATSEYQTFFNPR |
| 1645.8214 | 1645.7874 | −.034 | −21 | 385 | 399 | SPQELLCGASLI |
| | | | | | | SDR |
| 1698.7211 | 1698.6948 | −.0263 | −15 | 560 | 575 | RGDACEGDSG |
| | | | | | | GPFVMK |
| 1773.9164 | 1773.8727 | −.0437 | −25 | 384 | 399 | KSPQELLCGASL |
| | | | | | | ISDR |
| 1883.8956 | 1883.8428 | −.0528 | −28 | 328 | 344 | TFGSGEADCGL |
| | | | | | | RPLFEK |
| 1883.8956 | 1883.8428 | −.0528 | −28 | 328 | 344 | TFGSGEADCGL |
| | | | | | | RPLFEK |
| 1963.908 | 1963.848 | −.06 | −31 | 248 | 263 | HQDFNSAVQLV |
| | | | | | | ENFCR |
| 1968.8903 | 1968.8427 | −.0476 | −24 | 100 | 117 | LAACLEGNCAE |
| | | | | | | GLGTNYR |
| 2001.9158 | 2001.8715 | −.0443 | −22 | 544 | 560 | ITDNMFCAGYKP |
| | | | | | | DEGKR |
| 2011.9906 | 2011.9392 | −.0514 | −26 | 328 | 345 | TFGSGEADCGL |
| | | | | | | RPLFEK K |
| 2017.9106 | 2017.8978 | −.0128 | −6 | 544 | 560 | ITDNMFCAGYKP |
| | | | | | | DEGKR |
| 2070.9761 | 2070.9165 | −.0596 | −29 | 199 | 217 | SEGSSVNLSPPL |
| | | | | | | EQCVP |
| | | | | | | DR |
| 2200.9275 | 2200.9404 | .0129 | 6 | 159 | 177 | NPDSSTMGPWC |
| | | | | | | YTTDPT |
| | | | | | | VR |
| 2212.0122 | 2212.0205 | .0083 | 4 | 98 | 117 | DKLAACLEGNCA |
| | | | | | | EGLGT |
| | | | | | | NYR |
| 2212.0122 | 2212.0205 | .0083 | 4 | 98 | 117 | DKLAACLEGNCA |
| | | | | | | EGLGT |
| | | | | | | NYR |
| 2232.2534 | 2232.1794 | −.074 | −33 | 518 | 537 | GQPSVLQVVNLP |
| | | | | | | IVERPV |
| | | | | | | CK |
| 2232.2534 | 2232.1794 | −.074 | −33 | 518 | 537 | GQPSVLQVVNLP |
| | | | | | | IVERPV |
| | | | | | | CK |
| 2281.0991 | 2281.072 | −.0271 | −12 | 364 | 383 | IVEGSDAEIGMS |
| | | | | | | PWQVM |
| | | | | | | LFR |
| 2292.0417 | 2292.0005 | −.0412 | −18 | 179 | 198 | QECSIPVCGQDQ |
| | | | | | | VTVAM |
| | | | | | | TPR |
| 2448.1428 | 2448.1128 | −.03 | −12 | 178 | 198 | RQECSIPVCGQD |
| | | | | | | QVTVA MTPR |
| 2888.3591 | 2888.29 | −.0691 | −24 | 199 | 224 | SEGSSVNLSPPL |
| | | | | | | EQCVP |
| | | | | | | DRGQQYQGR |
| 2888.3591 | 2888.29 | −.0691 | −24 | 199 | 224 | SEGSSVNLSPPL |
| | | | | | | EQCVP |
| | | | | | | DRGQQYQGR |

| | |
|---|---|
| unnamed protein product [Homo sapiens] | gi\|194383496 |

Peptide Information

| | Obsrv. | | | Start | End | |
|---|---|---|---|---|---|---|
| Calc. Mass | Mass | ±da | ±ppm | Seq. | Seq. | Sequence |
| 836.4009 | 836.415 | .0141 | 17 | 201 | 207 | GQQYQGR |
| 836.4009 | 836.415 | .0141 | 17 | 201 | 207 | GQQYQGR |
| 874.4781 | 874.4888 | .0107 | 12 | 484 | 491 | VTGWGNLK |
| 951.4894 | 951.5774 | .088 | 92 | 417 | 423 | YERNIEK |
| 971.425 | 971.4321 | .0071 | 7 | 70 | 77 | YTACETAR |
| 1148.5518 | 1148.5477 | −.0041 | −4 | 108 | 116 | SGIECQLWR |
| 1148.5518 | 1148.5477 | −.0041 | −4 | 108 | 116 | SGIECQLWR |
| 1189.579 | 1189.5732 | −.0058 | −5 | 583 | 591 | YGFYTHVFR |
| 1194.6001 | 1194.5807 | −.0194 | −16 | 337 | 346 | ELLESYIDGR |
| 1194.6001 | 1194.5807 | −.0194 | −16 | 337 | 346 | ELLESYIDGR |
| 1265.6022 | 1265.592 | −.0102 | −8 | 436 | 444 | YNWRENLDR |
| 1489.7223 | 1489.7014 | −.0209 | −14 | 580 | 591 | DGKYGFYTHVFR |
| 1542.62 | 1542.6259 | .0059 | 4 | 544 | 558 | GDACEGDSGGPFV |
| | | | | | | MK |
| 1561.7281 | 1561.6975 | −.0306 | −20 | 298 | 310 | TATSEYQTFFNPR |
| 1561.7281 | 1561.6975 | −.0306 | −20 | 298 | 310 | TATSEYQTFFNPR |
| 1645.8214 | 1645.7874 | −.034 | −21 | 368 | 382 | SPQELLCGASLISDR |
| 1698.7211 | 1698.6948 | −.0263 | −15 | 543 | 558 | RGDACEGDSGG |
| | | | | | | PFVMK |
| 1773.9164 | 1773.8727 | −.0437 | −25 | 367 | 382 | KSPQELLCGASL |
| | | | | | | ISDR |
| 1883.8956 | 1883.8428 | −.0528 | −28 | 311 | 327 | TFGSGEADCGL |
| | | | | | | RPLFEK |
| 1883.8956 | 1883.8428 | −.0528 | −28 | 311 | 327 | TFGSGEADCGL |
| | | | | | | RPLFEK |
| 1963.908 | 1963.848 | −.06 | −31 | 231 | 246 | HQDFNSAVQLV |
| | | | | | | ENFCR |
| 1968.8903 | 1968.8427 | −.0476 | −24 | 83 | 100 | LAACLEGNCAEG |
| | | | | | | LGTNYR |
| 2001.9158 | 2001.8715 | −.0443 | −22 | 527 | 543 | ITDNMFCAGYKP |
| | | | | | | DEGKR |
| 2011.9906 | 2011.9392 | −.0514 | −26 | 311 | 328 | TFGSGEADCGL |
| | | | | | | RPLFEKK |
| 2017.9106 | 2017.8978 | −.0128 | −6 | 527 | 543 | ITDNMFCAGYKP |
| | | | | | | DEGKR |
| 2070.9761 | 2070.9165 | −.0596 | −29 | 182 | 200 | SEGSSVNLSPPL |
| | | | | | | EQCVP |
| | | | | | | DR |
| 2212.0122 | 2212.0205 | .0083 | 4 | 81 | 100 | DKLAACLEGNCA |
| | | | | | | EGLGT |
| | | | | | | NYR |
| 2212.0122 | 2212.0205 | .0083 | 4 | 81 | 100 | DKLAACLEGNCA |
| | | | | | | EGLGT |
| | | | | | | NYR |
| 2232.2534 | 2232.1794 | −.074 | −33 | 501 | 520 | GQPSVLQVVNLP |
| | | | | | | IVERPV |
| | | | | | | CK |
| 2232.2534 | 2232.1794 | −.074 | −33 | 501 | 520 | GQPSVLQVVNLP |
| | | | | | | IVERPV CK |
| 2281.0991 | 2281.072 | −.0271 | −12 | 347 | 366 | IVEGSDAEIGMS |
| | | | | | | PWQVM |
| | | | | | | LFR |
| 2292.0417 | 2292.0005 | −.0412 | −18 | 162 | 181 | QECSIPVCGQD |
| | | | | | | QVTVAM |
| | | | | | | TPR |
| 2448.1428 | 2448.1128 | −.03 | −12 | 161 | 181 | RQECSIPVCGQD |
| | | | | | | QVTVA |
| | | | | | | MTPR |
| 2888.3591 | 2888.29 | −.0691 | −24 | 182 | 207 | SEGSSVNLSPPL |
| | | | | | | EQCVP |
| | | | | | | DRGQQYQGR |
| 2888.3591 | 2888.29 | −.0691 | −24 | 182 | 207 | SEGSSVNLSPPL |
| | | | | | | EQCVP |
| | | | | | | DRGQQYQGR |

| | |
|---|---|
| unnamed protein product [Homo sapiens] | gi\|1335344 |

Protein Group

| | |
|---|---|
| coagulation factor II (thrombin), isoform CRA_a | gi\|119588383 |
| [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 836.4009 | 836.415 | .0141 | 17 | 211 | 217 | GQQYQGR |
| 836.4009 | 836.415 | .0141 | 17 | 211 | 217 | GQQYQGR |
| 874.4781 | 874.4888 | .0107 | 12 | 494 | 501 | VTGWGNLK |
| | 874.4888 | | | | | |
| 951.4894 | 951.5774 | .088 | 92 | 427 | 433 | YERNIEK |
| 971.425 | 971.4321 | .0071 | 7 | 80 | 87 | YTACETAR |
| 1148.5518 | 1148.5477 | −.0041 | −4 | 118 | 126 | SGIECQLWR |
| 1148.5518 | 1148.5477 | −.0041 | −4 | 118 | 126 | SGIECQLWR |
| 1189.579 | 1189.5732 | −.0058 | −5 | 593 | 601 | YGFYTHVFR |
| 1194.6001 | 1194.5807 | −.0194 | −16 | 347 | 356 | ELLESYIDGR |
| 1194.6001 | 1194.5807 | −.0194 | −16 | 347 | 356 | ELLESYIDGR |
| 1265.6022 | 1265.592 | −.0102 | −8 | 446 | 454 | YNWRENLDR |
| 1489.7223 | 1489.7014 | −.0209 | −14 | 590 | 601 | DGKYGFYTHVFR |
| 1542.62 | 1542.6259 | .0059 | 4 | 554 | 568 | GDACEGDSGGPFV |
| | | | | | | MK |
| 1561.7281 | 1561.6975 | −.0306 | −20 | 308 | 320 | TATSEYQTFFNPR |
| 1561.7281 | 1561.6975 | −.0306 | −20 | 308 | 320 | TATSEYQTFFNPR |
| 1645.8214 | 1645.7874 | −.034 | −21 | 378 | 392 | SPQELLCGASLISDR |
| 1698.7211 | 1698.6948 | −.0263 | −15 | 553 | 568 | RGDACEGDSGGPF |
| | | | | | | VMK |
| 1773.9164 | 1773.8727 | −.0437 | −25 | 377 | 392 | KSPQELLCGASLISDR |
| 1883.8956 | 1883.8428 | −.0528 | −28 | 321 | 337 | TFGSGEADCGLRPL |
| | | | | | | FEK |
| 1883.8956 | 1883.8428 | −.0528 | −28 | 321 | 337 | TFGSGEADCGLRPL |
| | | | | | | FEK |
| 1963.908 | 1963.848 | −.06 | −31 | 241 | 256 | HQDFNSAVQLVENF |
| | | | | | | CR |
| 1968.8903 | 1968.8427 | −.0476 | −24 | 93 | 110 | LAACLEGNCAEGLG |
| | | | | | | TNYR |
| 2001.9158 | 2001.8715 | −.0443 | −22 | 537 | 553 | ITDNMFCAGYKP |
| | | | | | | DEGKR |
| 2011.9906 | 2011.9392 | −.0514 | −26 | 321 | 338 | TFGSGEADCGL |
| | | | | | | RPLFEKK |
| 2017.9106 | 2017.8978 | −.0128 | −6 | 537 | 553 | ITDNMFCAGYKP |
| | | | | | | DEGKR |
| 2070.9761 | 2070.9165 | −.0596 | −29 | 192 | 210 | SEGSSVNLSPPL |
| | | | | | | EQCVP |
| | | | | | | DR |
| 2212.0122 | 2212.0205 | .0083 | 4 | 91 | 110 | DKLAACLEGNCA |
| | | | | | | EGLGT |
| | | | | | | NYR |
| 2212.0122 | 2212.0205 | .0083 | 4 | 91 | 110 | DKLAACLEGNCA |
| | | | | | | EGLGT |
| | | | | | | NYR |
| 2232.2534 | 2232.1794 | −.074 | −33 | 511 | 530 | GQPSVLQVVNLP |
| | | | | | | IVERPV |
| | | | | | | CK |
| 2232.2534 | 2232.1794 | −.074 | −33 | 511 | 530 | GQPSVLQVVNLP |
| | | | | | | IVERPV |
| | | | | | | CK |
| 2281.0991 | 2281.072 | −.0271 | −12 | 357 | 376 | IVEGSDAEIGMS |
| | | | | | | PWQVM |
| | | | | | | LFR |
| 2292.0417 | 2292.0005 | −.0412 | −18 | 172 | 191 | QECSIPVCGQD |
| | | | | | | QVTVAM |
| | | | | | | TPR |
| 2448.1428 | 2448.1128 | −.03 | −12 | 171 | 191 | RQECSIPVCGQD |
| | | | | | | QVTVA |
| | | | | | | MTPR |
| 2888.3591 | 2888.29 | −.0691 | −24 | 192 | 217 | SEGSSVNLSPPL |
| | | | | | | EQCVP |
| | | | | | | DRGQQYQGR |
| 2888.3591 | 2888.29 | −.0691 | −24 | 192 | 217 | SEGSSVNLSPPL |
| | | | | | | EQCVP |
| | | | | | | DRGQQYQGR |

| | |
|---|---|
| prothrombin preproprotein [Homo sapiens] | gi\|4503635 |

Protein Group

| | |
|---|---|
| Coagulation factor II (thrombin) [Homo sapiens] | gi\|30802115 |
| RecName: Full = Prothrombin; AltName: | gi\|135807 |
| Full = Coagulation factor II; Contains: | |
| RecName: Full = Activation peptide | |
| fragment 1; Contains: RecName: | |
| Full = Activation peptide fragment 2; | |
| Contains: RecName: Full = Thrombin | |
| light chain; | |
| Contains: RecName: Full = Thrombin | |
| heavy chain; Flags: | |
| Precursor | |
| coagulation factor II (thrombin) [Homo sapiens] | gi\|18653448 |
| coagulation factor II (thrombin), isoform CRA_c | gi\|119588385 |
| [Homo sapiens] | |
| coagulation factor II precursor variant [Homo sapiens] | gi\|62897113 |
| prothrombin [Homo sapiens] | gi\|339641 |
| prothrombin [Homo sapiens] | |
| prothrombin [Homo sapiens] | gi\|67624831 |
| unnamed protein product [Homo sapiens] | gi\|110623182 |

Peptide Information

| | Obsrv. | | | Start | End | |
|---|---|---|---|---|---|---|
| Calc. Mass | Mass | ±da | ±ppm | Seq. | Seq. | Sequence |
| 836.4009 | 836.415 | .0141 | 17 | 218 | 224 | GQQYQGR |
| 836.4009 | 836.415 | .0141 | 17 | 218 | 224 | GQQYQGR |
| 874.4781 | 874.4888 | .0107 | 12 | 501 | 508 | VTGWGNLK |
| 951.4894 | 951.5774 | .088 | 92 | 434 | 440 | YERNIEK |
| 971.425 | 971.4321 | .0071 | 7 | 87 | 94 | YTACETAR |
| 1148.5518 | 1148.5477 | −.0041 | −4 | 125 | 133 | SGIECQLWR |
| 1148.5518 | 1148.5477 | −.0041 | −4 | 125 | 133 | SGIECQLWR |
| 1189.579 | 1189.5732 | −.0058 | −5 | 600 | 608 | YGFYTHVFR |
| 1194.6001 | 1194.5807 | −.0194 | −16 | 354 | 363 | ELLESYIDGR |
| 1194.6001 | 1194.5807 | −.0194 | −16 | 354 | 363 | ELLESYIDGR |
| 1265.6022 | 1265.592 | −.0102 | −8 | 453 | 461 | YNWRENLDR |
| 1489.7223 | 1489.7014 | −.0209 | −14 | 597 | 608 | DGKYGFYTHVFR |
| 1542.62 | 1542.6259 | .0059 | 4 | 561 | 575 | GDACEGDSGGPFV |
| | | | | | | MK |
| 1561.7281 | 1561.6975 | −.0306 | −20 | 315 | 327 | TATSEYQTFFNPR |
| 1561.7281 | 1561.6975 | −.0306 | −20 | 315 | 327 | TATSEYQTFFNPR |
| 1645.8214 | 1645.7874 | −.034 | −21 | 385 | 399 | SPQELLCGASLISDR |
| 1698.7211 | 1698.6948 | −.0263 | −15 | 560 | 575 | RGDACEGDSGGPF |
| | | | | | | VMK |
| 1773.9164 | 1773.8727 | −.0437 | −25 | 384 | 399 | KSPQELLCGASL |
| | | | | | | ISDR |
| 1883.8956 | 1883.8428 | −.0528 | −28 | 328 | 344 | TFGSGEADCGL |
| | | | | | | RPLFEK |
| 1883.8956 | 1883.8428 | −.0528 | −28 | 328 | 344 | TFGSGEADCGL |
| | | | | | | RPLFEK |
| 1963.908 | 1963.848 | −.06 | −31 | 248 | 263 | HQDFNSAVQLV |
| | | | | | | ENFCR |
| 1968.8903 | 1968.8427 | −.0476 | −24 | 100 | 117 | LAACLEGNCAE |
| | | | | | | GLGTNYR |
| 2001.9158 | 2001.8715 | −.0443 | −22 | 544 | 560 | ITDNMFCAGYKP |
| | | | | | | DEGKR |
| 2011.9906 | 2011.9392 | −.0514 | −26 | 328 | 345 | TFGSGEADCGL |
| | | | | | | RPLFEKK |
| 2017.9106 | 2017.8978 | −.0128 | −6 | 544 | 560 | ITDNMFCAGYKP |
| | | | | | | DEGKR |
| 2070.9761 | 2070.9165 | −.0596 | −29 | 199 | 217 | SEGSSVNLSPPL |
| | | | | | | EQCVP |
| | | | | | | DR |
| 2212.0122 | 2212.0205 | .0083 | 4 | 98 | 117 | DKLAACLEGNCA |
| | | | | | | EGLGT |
| | | | | | | NYR |
| 2212.0122 | 2212.0205 | .0083 | 4 | 98 | 117 | DKLAACLEGNCA |
| | | | | | | EGLGT |
| | | | | | | NYR |
| 2232.2534 | 2232.1794 | −.074 | −33 | 518 | 537 | GQPSVLQVVNLP |
| | | | | | | IVERPV |
| | | | | | | CK |
| 2232.2534 | 2232.1794 | −.074 | −33 | 518 | 537 | GQPSVLQVVNLP |
| | | | | | | IVERPV |
| | | | | | | CK |
| 2281.0991 | 2281.072 | −.0271 | −12 | 364 | 383 | IVEGSDAEIGMS |
| | | | | | | PWQVM |
| | | | | | | LFR |
| 2292.0417 | 2292.0005 | −.0412 | −18 | 179 | 198 | QECSIPVCGQD |
| | | | | | | QVTVAM |
| | | | | | | TPR |
| 2448.1428 | 2448.1128 | −.03 | −12 | 178 | 198 | RQECSIPVCGQD |
| | | | | | | QVTVA |
| | | | | | | MTPR |
| 2888.3591 | 2888.29 | −.0691 | −24 | 199 | 224 | SEGSSVNLSPPL |
| | | | | | | EQCVP |
| | | | | | | DRGQQYQGR |
| 2888.3591 | 2888.29 | −.0691 | −24 | 199 | 224 | SEGSSVNLSPPL |
| | | | | | | EQCVP |
| | | | | | | DRGQQYQGR |

| | |
|---|---|
| coagulation factor II precursor variant | gi\|62897109 |
| [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 836.4009 | 836.415 | .0141 | 17 | 218 | 224 | GQQYQGR |
| 836.4009 | 836.415 | .0141 | 17 | 218 | 224 | GQQYQGR |
| 874.4781 | 874.4888 | .0107 | 12 | 501 | 508 | VTGWGNLK |
| 951.4894 | 951.5774 | .088 | 92 | 434 | 440 | YERNIEK |
| 971.425 | 971.4321 | .0071 | 7 | 87 | 94 | YTACETAR |
| 1148.5518 | 1148.5477 | −.0041 | −4 | 125 | 133 | SGIECQLWR |
| 1148.5518 | 1148.5477 | −.0041 | −4 | 125 | 133 | SGIECQLWR |
| 1189.579 | 1189.5732 | −.0058 | −5 | 600 | 608 | YGFYTHVFR |
| 1194.6001 | 1194.5807 | −.0194 | −16 | 354 | 363 | ELLESYIDGR |
| 1194.6001 | 1194.5807 | −.0194 | −16 | 354 | 363 | ELLESYIDGR |
| 1265.6022 | 1265.592 | −.0102 | −8 | 453 | 461 | YNWRENLDR |
| 1489.7223 | 1489.7014 | −.0209 | −14 | 597 | 608 | DGKYGFYTHVFR |
| 1542.62 | 1542.6259 | .0059 | 4 | 561 | 575 | GDACEGDSGG |
| | | | | | | PFVMK |
| 1561.7281 | 1561.6975 | −.0306 | −20 | 315 | 327 | TATSEYQTFFN |
| | | | | | | PR |
| 1561.7281 | 1561.6975 | −.0306 | −20 | 315 | 327 | TATSEYQTFFN |
| | | | | | | PR |
| 1645.8214 | 1645.7874 | −.034 | −21 | 385 | 399 | SPQELLCGASLI |
| | | | | | | SDR |
| 1698.7211 | 1698.6948 | −.0263 | −15 | 560 | 575 | RGDACEGDSGG |
| | | | | | | PFVMK |
| 1773.9164 | 1773.8727 | −.0437 | −25 | 384 | 399 | KSPQELLCGASL |
| | | | | | | ISDR |
| 1963.908 | 1963.848 | −.06 | −31 | 248 | 263 | HQDFNSAVQLV |
| | | | | | | ENFCR |
| 1968.8903 | 1968.8427 | −.0476 | −24 | 100 | 117 | LAACLEGNCAEG |
| | | | | | | LGTNYR |
| 2001.9158 | 2001.8715 | −.0443 | −22 | 544 | 560 | ITDNMFCAGYKP |
| | | | | | | DEGKR |
| 2017.9106 | 2017.8978 | −.0128 | −6 | 544 | 560 | ITDNMFCAGYKP |
| | | | | | | DEGKR |
| 2070.9761 | 2070.9165 | −.0596 | −29 | 199 | 217 | SEGSSVNLSPPL |
| | | | | | | EQCVP |
| | | | | | | DR |
| 2212.0122 | 2212.0205 | .0083 | 4 | 98 | 117 | DKLAACLEGNCA |
| | | | | | | EGLGT |
| | | | | | | NYR |
| 2212.0122 | 2212.0205 | .0083 | 4 | 98 | 117 | DKLAACLEGNCA |
| | | | | | | EGLGT |
| | | | | | | NYR |
| 2232.2534 | 2232.1794 | −.074 | −33 | 518 | 537 | GQPSVLQVVNLP |
| | | | | | | IVERPV |
| | | | | | | CK |
| 2232.2534 | 1232.1794 | −.074 | −33 | 518 | 537 | GQPSVLQVVNLP |
| | | | | | | IVERPV |
| | | | | | | CK |
| 2281.0991 | 2281.072 | −.0271 | −12 | 364 | 383 | IVEGSDAEIGMS |
| | | | | | | PWQVM |
| | | | | | | LFR |
| 2292.0417 | 2292.0005 | −.0412 | −18 | 179 | 198 | QECSIPVCGQD |
| | | | | | | QVTVAM |
| | | | | | | TPR |
| 2448.1428 | 2448.1128 | −.03 | −12 | 178 | 198 | RQECSIPVCGQD |
| | | | | | | QVTVA |
| | | | | | | MTPR |
| 2888.3591 | 2888.29 | −.0691 | −24 | 199 | 224 | SEGSSVNLSPPL |
| | | | | | | EQCVP |
| | | | | | | DRGQQYQGR |
| 2888.3591 | 2888.29 | −.0691 | −24 | 199 | 224 | SEGSSVNLSPPL |
| | | | | | | EQCVP |
| | | | | | | DRGQQYQGR |

| | |
|---|---|
| coagulation factor II (thrombin), isoform CRA_b | gi\|119588384 |
| [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 836.4009 | 836.415 | .0141 | 17 | 218 | 224 | GQQYQGR |
| 836.4009 | 836.415 | .0141 | 17 | 218 | 224 | GQQYQGR |
| 874.4781 | 874.4888 | .0107 | 12 | 501 | 508 | VTGWGNLK |
| 951.4894 | 951.5774 | .088 | 92 | 434 | 440 | YERNIEK |
| 971.425 | 971.4321 | .0071 | 7 | 87 | 94 | YTACETAR |
| 1148.5518 | 1148.5477 | −.0041 | −4 | 125 | 133 | SGIECQLWR |
| 1148.5518 | 1148.5477 | −.0041 | −4 | 125 | 133 | SGIECQLWR |
| 1194.6001 | 1194.5807 | −.0194 | −16 | 354 | 363 | ELLESYIDGR |
| | 1194.5807 | | | | | |
| 1194.6001 | 1194.5807 | −.0194 | −16 | 354 | 363 | ELLESYIDGR |
| 1265.6022 | 1265.592 | −.0102 | −8 | 453 | 461 | YNWRENLDR |
| 1561.7281 | 1561.6975 | −.0306 | −20 | 315 | 327 | TATSEYQTFFNPR |
| 1561.7281 | 1561.6975 | −.0306 | −20 | 315 | 327 | TATSEYQTFFNPR |
| 1645.8214 | 1645.7874 | −.034 | −21 | 385 | 399 | SPQELLCGASLI |
| | | | | | | SDR |
| 1773.9164 | 1773.8727 | −.0437 | −25 | 384 | 399 | KSPQELLCGASL |
| | | | | | | ISDR |
| 1883.8956 | 1883.8428 | −.0528 | −28 | 328 | 344 | TFGSGEADCGL |
| | | | | | | RPLFEK |
| 1883.8956 | 1883.8428 | −.0528 | −28 | 328 | 344 | TFGSGEADCGL |
| | | | | | | RPLFEK |
| 1963.908 | 1963.848 | −.06 | −31 | 248 | 263 | HQDFNSAVQLV |
| | | | | | | ENFCR |
| 1968.8903 | 1968.8427 | −.0476 | −24 | 100 | 117 | LAACLEGNCAEG |
| | | | | | | LGTNYR |
| 2011.9906 | 2011.9392 | −.0514 | −26 | 328 | 345 | TFGSGEADCGL |
| | | | | | | RPLFEKK |
| 2070.9761 | 2070.9165 | −.0596 | −29 | 199 | 217 | SEGSSVNLSPPL |
| | | | | | | EQCVP |
| | | | | | | DR |
| 2212.0122 | 2212.0205 | .0083 | 4 | 98 | 117 | DKLAACLEGNCA |
| | | | | | | EGLGT |
| | | | | | | NYR |
| 2212.0122 | 2212.0205 | .0083 | 4 | 98 | 117 | DKLAACLEGNCA |
| | | | | | | EGLGT |
| | | | | | | NYR |
| 2232.2534 | 2232.1794 | −.074 | −33 | 518 | 537 | GQPSVLQVVNLP |
| | | | | | | IVERPV |
| | | | | | | CK |
| 2232.2534 | 2232.1794 | −.074 | −33 | 518 | 537 | GQPSVLQVVNLP |
| | | | | | | IVERPV |
| | | | | | | CK |
| 2281.0991 | 2281.072 | −.0271 | −12 | 364 | 383 | IVEGSDAEIGMS |
| | | | | | | PWQVM |
| | | | | | | LFR |
| 2292.0417 | 2292.0005 | −.0412 | −18 | 179 | 198 | QECSIPVCGQD |
| | | | | | | QVTVAM |
| | | | | | | TPR |
| 2448.1428 | 2448.1128 | −.03 | −12 | 178 | 198 | RQECSIPVCGQD |
| | | | | | | QVTVA |
| | | | | | | MTPR |
| 2888.3591 | 2888.29 | −.0691 | −24 | 199 | 224 | SEGSSVNLSPPL |
| | | | | | | EQCVP |
| | | | | | | DRGQQYQGR |
| 2888.3591 | 2888.29 | −.0691 | −24 | 199 | 224 | SEGSSVNLSPPL |
| | | | | | | EQCVP |
| | | | | | | DRGQQYQGR |

| | |
|---|---|
| unnamed protein product [Homo sapiens] | gi\|194383358 |
| Full = PHD-like zinc finger protein | |
| unnamed protein product [Homo sapiens] | |
| unnamed protein product [Homo sapiens] | gi\|194382512 |

Peptide Information

| | Obsrv. | | | Start | End | |
|---|---|---|---|---|---|---|
| Calc. Mass | Mass | ±da | ±ppm | Seq. | Seq. | Sequence |
| 928.4193 | 928.4557 | .0364 | 39 | 314 | 320 | YIENMSR |

| | |
|---|---|
| zinc finger, CCHC domain | gi\|119582703 |
| containing 10, isoform | |
| CRA_d [Homo sapiens] | |

Protein Group

| | |
|---|---|
| RecName: Full = Zinc finger CCHC domain-containing | gi\|84028163 |
| protein 10 | |
| ZCCHC10 protein [Homo sapiens] | gi\|18490660 |
| Zinc finger, CCHC domain containing 10 [Homo | gi\|13528822 |
| sapiens] | |
| unnamed protein product [Homo sapiens] | gi\|194373877 |
| unnamed protein product [Homo sapiens] | gi\|193784674 |
| unnamed protein product [Homo sapiens] | gi\|7019971 |
| unnamed protein product [Homo sapiens] | gi\|40043232 |
| unnamed protein product [Homo sapiens] | gi\|40977976 |
| zinc finger CCHC domain-containing protein 10 [Homo | gi\|8923106 |
| sapiens] | |
| zinc finger, CCHC domain containing 10, isoform | gi\|119582700 |
| CRA_a [Homo sapiens] | |
| zinc finger, CCHC domain containing 10, isoform | gi\|119582701 |
| CRA_b [Homo sapiens] | |
| zinc finger, CCHC domain containing 10, isoform | gi\|119582702 |
| CRA_c [Homo sapiens] | |
| zinc finger, CCHC domain containing 10, isoform | gi\|119582704 |
| CRA_e [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 928.5111 | 928.4557 | −.0554 | −60 | 64 | 70 | YLHRPSR |

| | |
|---|---|
| class E basic helix-loop-helix | gi\|13540521 |
| protein 41 [Homo sapiens] | |

Protein Group

| | |
|---|---|
| Basic helix-loop-helix family, member e41 [Homo | gi\|19684064 |
| sapiens] | |
| RecName: Full = Class E basic helix-loop-helix protein | gi\|20137459 |
| 41; Short = bHLHe41; AltName: Full = Class | |
| B basic helix-loop-helix protein 3; | |
| Short = bHLHb3; AltName: Full = Differentially | |
| expressed in chondrocytes protein 2; | |
| Short = hDEC2; AltName: Full = Enhancer-of-split and | |
| hairy-related protein 1; Short = SHARP-1 | |
| bHLH protein DEC2 [Homo sapiens] | gi\|12583669 |
| basic helix-loop-helix domain containing, class B, 3, | gi\|119616934 |
| isoform CRA_a [Homo sapiens] | |
| basic helix-loop-helix domain containing, class B, 3, | gi\|119616933 |
| isoform CRA_a [Homo sapiens] | |
| basic helix-loop-helix domain-containing [Homo | gi\|121647001 |
| sapiens] | |
| basic helix-loop-helix protein 3 isoform 1 [Homo | gi\|55669468 |
| sapiens] | |
| unnamed protein product [Homo sapiens] | gi\|221044670 |

Peptide Information

| | Obsrv. | | | Start | End | |
|---|---|---|---|---|---|---|
| Calc. Mass | Mass | ±da | ±ppm | Seq. | Seq. | Sequence |
| 1340.6263 | 1340.6824 | .0561 | 42 | 1 | 11 | MDEGIPHLQER |

| | |
|---|---|
| RecName: Full = Alkaline phosphatase, | gi\|68067533 |
| tissue-nonspecific isozyme; Short = | |
| AP-TNAP; Short = TNSALP; | |
| AltName: Full = Alkaline phosphatase | |
| liver/bone/kidney isozyme; | |
| Flags: Precursor | |

Protein Group

| | |
|---|---|
| Alkaline phosphatase, liver/bone/kidney [Homo sapiens] | gi\|187954299 |
| Alkaline phosphatase, liver/bone/kidney [Homo sapiens] | gi\|127801853 |
| Alkaline phosphatase, liver/bone/kidney [Homo sapiens] | gi\|127799935 |
| Alkaline phosphatase, liver/bone/kidney [Homo sapiens] | gi\|127799243 |
| Alkaline phosphatase, liver/bone/kidney [Homo sapiens] | gi\|118835602 |
| Alkaline phosphatase, liver/bone/kidney [Homo sapiens] | gi\|127796485 |
| alkalin phosphatase [Homo sapiens] | gi\|3401945 |
| alkaline phosphatase [Homo sapiens] | gi\|178462 |
| alkaline phosphatase, liver/bone/kidney [Homo sapiens] | gi\|55959694 |
| alkaline phosphatase, liver/bone/kidney [Homo sapiens] | gi\|55665927 |
| alkaline phosphatase, liver/bone/kidney, isoform | gi\|119615382 |
| CRA_a [Homo sapiens] | |
| alkaline phosphatase, liver/bone/kidney, isoform | gi\|119615381 |
| CRA_a [Homo sapiens] | |
| alkaline phosphatase, liver/bone/kidney, isoform | gi\|119615380 |
| CRA_a [Homo sapiens] | |
| alkaline phosphatase, liver/bone/kidney, isoform | gi\|119615383 |
| CRA_b [Homo sapiens] | |
| alkaline phosphatase, tissue-nonspecific isozyme | gi\|116734717 |
| isoform 1 precursor [Homo sapiens] | |
| alkaline phosphatase, tissue-nonspecific isozyme | gi\|294660770 |
| isoform 2 [Homo sapiens] | |
| alkaline phosphatase, tissue-nonspecific isozyme | gi\|294660772 |
| isoform 3 [Homo sapiens] | |
| unnamed protein product [Homo sapiens] | gi\|221040844 |
| unnamed protein product [Homo sapiens] | gi\|28738 |
| unnamed protein product [Homo sapiens] | gi\|221042090 |
| unnamed protein product [Homo sapiens] | gi\|189069211 |
| unnamed protein product [Homo sapiens] | gi\|221039408 |

Peptide Information

| | Obsrv. | | | | | |
|---|---|---|---|---|---|---|
| Calc. Mass | Mass | ±da | ±ppm | Start Seq. | End Seq. | Sequence |
| 1835.9028 | 1835.9573 | .0545 | 30 | 137 | 152 | SRCNTTQGNEV |
| | | | | | | TSILR |

| | |
|---|---|
| eukaryotic translation initiation factor 3 | gi\|312433975 |
| subunit C [Homo sapiens] | |

Protein Group

| | |
|---|---|
| EIF3CL protein [Homo sapiens] | gi\|187956832 |
| Eukaryotic translation initiation factor 3, subunit C | gi\|187957270 |
| [Homo sapiens] | |
| Eukaryotic translation initiation factor 3, subunit C | gi\|16306765 |
| [Homo sapiens] | |
| Eukaryotic translation initiation factor 3, subunit C | gi\|47939099 |
| [Homo sapiens] | |
| RecName: Full = Eukaryotic translation initiation | gi\|6685539 |
| factor 3 subunit C; Short = eIF3c; AltName: | |
| Full = Eukaryotic translation initiation factor 3 | |
| subunit 8; AltName: Full = eIF3 p110 | |
| Translation initiation factor eIF-p110 [Homo sapiens] | gi\|3337383 |
| cell migration-inducing protein 17 [Homo sapiens] | gi\|59803473 |
| eIF-3 p110 subunit [Homo sapiens] | gi\|1931584 |
| eukaryotic translation initiation factor 3 subunit C [Homo | gi\|153791492 |
| sapiens] | |
| eukaryotic translation initiation factor 3 subunit C [Homo | gi\|83700233 |
| sapiens] | |
| eukaryotic translation initiation factor 3 subunit C [Homo | gi\|4503525 |
| sapiens] | |
| eukaryotic translation initiation factor 3, subunit 8, | gi\|119570881 |
| 110 kDa, isoform CRA_a [Homo sapiens] | |
| eukaryotic translation initiation factor 3, subunit 8, | gi\|119570877 |
| 110 kDa, isoform CRA_a [Homo sapiens] | |
| eukaryotic translation initiation factor 3, subunit 8, | gi\|119570879 |
| 110 kDa, isoform CRA_a [Homo sapiens] | |
| eukaryotic translation initiation factor 3, subunit 8, | gi\|119570880 |
| 110 kDa, isoform CRA_b [Homo sapiens] | |
| eukaryotic translation initiation factor 3, subunit 8, | gi\|119570878 |
| 110 kDa, isoform CRA_b [Homo sapiens] | |
| translation intiation factor eIF-3 p110 subunit [Homo | gi\|1718197 |
| sapiens] | |
| unnamed protein product [Homo sapiens] | gi\|194385866 |
| unnamed protein product [Homo sapiens] | gi\|194383552 |
| unnamed protein product [Homo sapiens] | gi\|194376092 |
| unnamed protein product [Homo sapiens] | gi\|194375309 |
| unnamed protein product [Homo sapiens] | gi\|194373399 |
| unnamed protein product [Homo sapiens] | gi\|7021015 |
| unnamed protein product [Homo sapiens] | gi\|158257742 |
| unnamed protein product [Homo sapiens] | gi\|194387978 |
| unnamed protein product [Homo sapiens] | gi\|193786223 |

Peptide Information

| Calc. | Obsrv. | | | Start | End | |
|---|---|---|---|---|---|---|
| Mass | Mass | ±da | ±ppm | Seq. | Seq. | Sequence |
| 928.4886 | 928.4557 | −.0329 | −35 | 554 | 560 | YIYAKDR |

| | |
|---|---|
| BarH-like homeobox 1 [Homo sapiens] | gi\|219521562 |

Peptide Information

| | Obsrv. | | | Start | End | |
|---|---|---|---|---|---|---|
| Calc. Mass | Mass | ±da | ±ppm | Seq. | Seq. | Sequence |
| 928.4886 | 928.4557 | −.0329 | −35 | 202 | 208 | YLSVQYR |

| | |
|---|---|
| eukaryotic translation initiation | gi\|119598673 |
| factor 4 gamma, 1, | |
| isoform CRA_g [Homo sapiens] | |

Protein Group

| | |
|---|---|
| EIF4G1 protein [Homo sapiens] | gi\|187956781 |
| EIF4G1 protein [Homo sapiens] | gi\|187954575 |
| EIF4G1 protein [Homo sapiens] | gi\|14043628 |
| EIF4G1 variant protein [Homo sapiens] | gi\|68533081 |
| RecName: Full = Eukaryotic translation initiation | gi\|294862538 |
| factor 4 gamma 1; Short = eIF-4-gamma 1; | |
| Short = eIF-4G 1; Short = eIF-4G1; AltName: | |
| Full = p220 | |
| eukaryotic protein synthesis initiation factor [Homo | gi\|3941724 |
| sapiens] | |
| eukaryotic translation initiation factor 4 gamma 1 | gi\|302699237 |
| isoform 1 [Homo sapiens] | |
| eukaryotic translation initiation factor 4 gamma 1 | gi\|38201627 |
| isoform 2 [Homo sapiens] | |
| eukaryotic translation initiation factor 4 gamma 1 | gi\|38201625 |
| isoform 3 [Homo sapiens] | |
| eukaryotic translation initiation factor 4 gamma 1 | gi\|302699239 |
| isoform 4 [Homo sapiens] | |
| eukaryotic translation initiation factor 4 gamma 1 | gi\|38201623 |
| isoform 5 [Homo sapiens] | |
| eukaryotic translation initiation factor 4 gamma 1 | gi\|302699247 |
| isoform 6 [Homo sapiens] | |
| eukaryotic translation initiation factor 4 gamma 1 | gi\|302699245 |
| isoform 6 [Homo sapiens] | |
| eukaryotic translation initiation factor 4 gamma, 1 | gi\|30583403 |
| [Homo sapiens] | |
| eukaryotic translation initiation factor 4 gamma, 1, | gi\|119598663 |
| isoform CRA_a [Homo sapiens] | |
| eukaryotic translation initiation factor 4 gamma, 1, | gi\|119598672 |
| isoform CRA_c [Homo sapiens] | |
| eukaryotic translation initiation factor 4 gamma, 1, | gi\|119598669 |
| isoform CRA_c [Homo sapiens] | |
| eukaryotic translation initiation factor 4 gamma, 1, | gi\|119598665 |
| isoform CRA_c [Homo sapiens] | |
| eukaryotic translation initiation factor 4 gamma, 1, | gi\|119598666 |
| isoform CRA_d [Homo sapiens] | |
| eukaryotic translation initiation factor 4 gamma, 1, | gi\|119598667 |
| isoform CRA_e [Homo sapiens] | |
| eukaryotic translation initiation factor 4 gamma, 1, | gi\|119598671 |
| isoform CRA_f [Homo sapiens] | |
| eukaryotic translation initiation factor 4 gamma, 1, | gi\|119598668 |
| isoform CRA_f [Homo sapiens] | |
| eukaryotic translation initiation factor 4 gamma, 1, | gi\|119598670 |
| isoform CRA_g [Homo sapiens] | |
| eukaryotic translation initiation factor 4 gamma, 1, | gi\|119598674 |
| isoform CRA_h [Homo sapiens] | |
| eukaryotic translation initiation factor 4G I [Homo | gi\|21634449 |
| sapiens] | |
| eukaryotic translation initiation factor 4GI [Homo | gi\|21655146 |
| sapiens] | |
| hypothetical protein [Homo sapiens] | gi\|57997536 |
| unnamed protein product [Homo sapiens] | gi\|194389380 |
| unnamed protein product [Homo sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 1340.7355 | 1340.6824 | −.0531 | −40 | 929 | 939 | EHIKVQQLMAK |

| | |
|---|---|
| exocyst complex component 6, isoform CRA_d | gi\|119570470 |
| [Homo sapiens] | |

Protein Group

| | |
|---|---|
| Exocyst complex component 6 [Homo sapiens] | gi\|34783239 |
| RecName: Full = Exocyst complex component 6; | gi\|212287926 |
| AltName: Full = Exocyst complex component | |
| Sec15A; AltName: Full = SEC15-like protein 1 | |
| SEC15-like protein 3 [Homo sapiens] | gi\|148608033 |
| exocyst complex component 6 [Homo sapiens] | gi\|123231220 |
| exocyst complex component 6 [Homo sapiens] | gi\|55665929 |
| exocyst complex component 6 [Homo sapiens] | gi\|123227366 |
| exocyst complex component 6 isoform a [Homo | gi\|65507403 |
| sapiens] | |
| exocyst complex component 6 isoform b [Homo | gi\|62243652 |
| sapiens] | |
| exocyst complex component 6, isoform CRA_a [Homo | gi\|119570467 |
| sapiens] | |
| exocyst complex component 6, isoform CRA_b [Homo | gi\|119570468 |
| sapiens] | |
| exocyst complex component 6, isoform CRA_c [Homo | gi\|119570469 |
| sapiens] | |
| exocyst complex component 6, isoform CRA_e [Homo | gi\|119570471 |
| sapiens] | |
| hypothetical protein [Homo sapiens] | gi\|6807998 |
| unnamed protein product [Homo sapiens] | gi\|221043148 |
| unnamed protein product [Homo sapiens] | gi\|194375816 |
| unnamed protein product [Homo sapiens] | gi\|40032933 |
| unnamed protein product [Homo sapiens] | gi\|108250166 |
| unnamed protein product [Homo sapiens] | gi\|193785860 |
| unnamed protein product [Homo sapiens] | gi\|189054938 |
| unnamed protein product [Homo sapiens] | gi\|7023795 |
| unnamed protein product [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 928.5138 | 928.4557 | −.0581 | −63 | 583 | 590 | LYGLSTFK |

| | |
|---|---|
| RecName: Full = Protein FAM183B | gi\|215275260 |

Protein Group

| | |
|---|---|
| hCG1645872 [Homo sapiens] | gi\|119614518 |
| hypothetical gene supported by BC045803 [Homo | gi\|51094744 |
| sapiens] | |
| testis highly expressed protein 6 [Homo sapiens] | gi\|225734648 |
| unnamed protein product [Homo sapiens] | gi\|261385756 |
| unnamed protein product [Homo sapiens] | gi\|40046854 |

Peptide Information

| | Obsrv. | | | Start | End | |
|---|---|---|---|---|---|---|
| Calc. Mass | Mass | ±da | ±ppm | Seq. | Seq. | Sequence |
| 1835.9723 | 1835.9573 | −.015 | −8 | 7 | 21 | ERVVTDEVHQNQILR |

| | |
|---|---|
| SWI/SNF related, matrix associated, actin | gi\|119579218 |
| dependent regulator of chromatin, subfamily | |
| a, member 2, isoform | |
| CRA_a [Homo sapiens] | |
| HBRM [Homo sapiens] | gi\|414117 |

Protein Group

| | |
|---|---|
| RecName: Full = Probable global transcription activator | gi\|212276472 |
| SNF2L2; AltName: Full = ATP-dependent helicase | |
| SMARCA2; AltName: Full = BRG1-associated factor | |
| 190B; Short = BAF190B; AltName: | |
| Full = Protein brahma homolog; | |
| Short = hBRM; AltName: Full = SNF2-alpha; | |
| AltName: Full = SWI/SNF-related matrix- | |
| associated actin-dependent regulator of | |
| chromatin subfamily A | |
| member 2 | |
| SWI/SNF related, matrix associated, actin dependent | gi\|55957478 |
| regulator of chromatin, subfamily a, member 2 [Homo | |
| sapiens] | |
| SWI/SNF related, matrix associated, actin dependent | gi\|55958984 |
| regulator of chromatin, subfamily a, member 2 [Homo | |
| sapiens] | |
| SWI/SNF related, matrix associated, actin dependent | gi\|55958983 |
| regulator of chromatin, subfamily a, member 2 [Homo | |
| sapiens] | |
| SWI/SNF related, matrix associated, actin dependent | gi\|55957477 |
| regulator of chromatin, subfamily a, member 2 [Homo | |
| sapiens] | |
| SWI/SNF related, matrix associated, actin dependent | gi\|55958982 |
| regulator of chromatin, subfamily a, | |
| member 2 [Homo sapiens] | |
| regulator of chromatin, subfamily a, | |
| CRA a [Homo sapiens] | |
| SWI/SNF related, matrix associated, | gi\|119579 |
| regulator of chromatin, subfamily a, | |
| CRA c [Homo sapiens] | |
| SWI/SNF related, matrix associated, | gi\|119579 |
| regulator of chromatin, subfamily a, | |
| CRA c [Homo sapiens] | |
| probable global transcription activator | gi\|482559 |
| a [Homo sapiens] | |
| b [Homo sapiens] | |

| | | | | Start | | |
|---|---|---|---|---|---|---|
| Calc. Mass | Obsrv. Mass | ±da | ±ppm | Seq. | End Seq. | Sequence |
| 1405.7039 | 1405.7631 | .0592 | 42 | 391 | 401 | QLRQEVVACMR |

| | |
|---|---|
| C protein [Homo sapiens] | gi\|36501 |

Peptide Information

| | Obsrv. | | | Start | End | |
|---|---|---|---|---|---|---|
| Calc. Mass | Mass | ±da | ±ppm | Seq. | Seq. | Sequence |
| 949.4924 | 949.515 | .0226 | 24 | 972 | 978 | KSMEWLR |

Protein Group

| | |
|---|---|
| transducin-like enhancer of split 4 | gi\|55959926 |
| (E(sp1) homolog, | |
| Drosophila) [Homo sapiens] | |
| transducin-like enhancer of split 4 | gi\|55958795 |
| (E(sp1) homolog, | |
| Drosophila) [Homo sapiens] | |
| transducin-like enhancer of split 4 | gi\|57208918 |
| (E(sp1) homolog, | |
| Drosophila) [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 850.4417 | 850.405 | −.0367 | −43 | 198 | 205 | VSPSASFR |

| | |
|---|---|
| leucine rich repeat containing 27, | gi\|119569507 |
| isoform CRA_b | |
| [Homo sapiens] | |

Protein Group

| | |
|---|---|
| KIAA1674 protein [Homo sapiens] | gi\|12697893 |
| RecName: Full = Leucine-rich repeat-containing protein | gi\|85701139 |
| 27 | |
| leucine rich repeat containing 27, isoform CRA_a | gi\|119569506 |
| [Homo sapiens] | |
| leucine rich repeat containing 27, isoform CRA_a | gi\|119569508 |
| [Homo sapiens] | |
| leucine-rich repeat-containing protein 27 isoform a | gi\|219555700 |
| [Homo sapiens] | |
| leucine-rich repeat-containing protein 27 isoform a | gi\|55741567 |
| [Homo sapiens] | |
| novel protein [Homo sapiens] | gi\|55960576 |
| novel protein [Homo sapiens] | gi\|55960574 |
| novel protein [Homo sapiens] | |
| unnamed protein product [Homo sapiens] | gi\|194376382 |
| unnamed protein product [Homo sapiens] | gi\|193783556 |
| unnamed protein product [Homo sapiens] | gi\|21752065 |

Peptide Information

| | Obsrv. | | | | | |
|---|---|---|---|---|---|---|
| Calc. Mass | Mass | ±da | ±ppm | Start Seq. | End Seq. | Sequence |
| 1340.6852 | 1340.6824 | −.0028 | −2 | 381 | 390 | QHVLQMREQR |

### 3—Prothrombin Complex Concentrate

Below is the sequence of the 8 new found proteins which will be named KH11, KH12, KH13, KH14, KH15, KH16, KH17 and KH18 and its functions are under investigation.

| | | | |
|---|---|---|---|
| | 60/C9 | | Instr./Gel Origin |
| | [1] Sample Project | | Instrument |
| | 20110503 | | Sample |
| | | | Name |
| Protein | | | |
| Name- | KH11 (SEQ ID NO. KH11) | Accession No. | |

| | |
|---|---|
| unnamed protein product | gi\|215415638 |
| [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1311.5555 | 1311.5947 | .0392 | 30 | 78 | 88 | ETEGLCQEMSK |
| 1318.642 | 1318.6689 | .0269 | 20 | 141 | 151 | LSPLGEEMRDR |
| 1327.5505 | 1327.6265 | .076 | 57 | 78 | 88 | ETEGLCQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQF |
| | | | | | | EGSALGK |
| 2202.1189 | 2202.0879 | −.031 | −14 | 60 | 77 | LREQLGPVTQEFWDNLEK |

| | |
|---|---|
| Chain A, Solution Structure Of Double Super Helix | gi\|293651900 |
| Model | |

Protein Group

| | |
|---|---|
| Chain A, Crystal Structure Of Lipid-Free Human | gi\|90108664 |
| Apolipoprotein A-I | |
| Chain B, Crystal Structure Of Lipid-Free Human | gi\|90108665 |
| Apolipoprotein A-I | |
| Chain B, Solution Structure Of Double Super Helix | gi\|293651901 |
| Model | |
| Chain C, Crystal Structure Of Lipid-Free Human | gi\|90108666 |
| Apolipoprotein A-I | |
| apoA1 [Homo sapiens] | gi\|490098 |
| apoAI [Homo sapiens] | gi\|1330306 |
| unnamed protein product [Homo sapiens] | gi\|215415678 |

Peptide Information

| | | | | | End | |
|---|---|---|---|---|---|---|
| Calc. Mass | Obsrv. Mass | ±da | ±ppm | Start Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDF |
| | | | | | | QKK |
| 1386.7152 | 1386.7365 | .0213 | 15 | 227 | 238 | VSFLSALEE |
| | | | | | | YTK |
| 1400.6693 | 1400.715 | .0457 | 33 | 28 | 40 | DYVSQFEG |
| | | | | | | SALGK |
| 1411.6675 | 1411.6667 | −.0008 | −1 | 107 | 116 | KWQEEMEL |
| | | | | | | YR |
| 1453.707 | 1453.7174 | .0104 | 7 | 1 | 12 | DEPPQSPW |
| | | | | | | DRVK |
| 1467.7914 | 1467.7849 | −.0065 | −4 | 119 | 131 | VEPLRAELQEG |
| | | | | | | AR |
| 1467.7914 | 1467.7849 | −.0065 | −4 | 119 | 131 | VEPLRAELQEG |
| | | | | | | AR |
| 1585.8081 | 1585.8174 | .0093 | 6 | 161 | 173 | THLAPYSDELR |
| | | | | | | QR |
| 1585.8081 | 1585.8174 | .0093 | 6 | 161 | 173 | THLAPYSDELR |
| | | | | | | QR |
| 1612.7854 | 1612.8048 | .0194 | 12 | 46 | 59 | LLDNWDSVTST |
| | | | | | | FSK |
| 1650.8698 | 1650.8733 | .0035 | 2 | 13 | 27 | DLATVYVDVLK |
| | | | | | | DSGR |
| 1815.8508 | 1815.8446 | −.0062 | −3 | 24 | 40 | DSGRDYVSQF |
| | | | | | | EGSALGK |
| 1815.8508 | 1815.8446 | −.0062 | −3 | 24 | 40 | DSGRDYVSQF |
| | | | | | | EGSALGK |
| 2202.1189 | 2202.0879 | −.031 | −14 | 60 | 77 | LREQLGPVTQE |
| | | | | | | FWDNLEK |

| | |
|---|---|
| unnamed protein product [Homo sapiens] | gi\|215415636 |

Peptide Information

| | Obsrv. | | | | End | |
|---|---|---|---|---|---|---|
| Calc. Mass | Mass | ±da | ±ppm | Start Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| | 1585.8174 | | | | | |
| 1612.7854 | 1612.8048 | .0194 | 12 | 46 | 59 | LLDNWDSVTST |
| | | | | | | FSK |
| 1650.8698 | 1650.8733 | .0035 | 2 | 13 | 27 | DLATVYVDVLK |
| | | | | | | DSGR |
| 1815.8508 | 1815.8446 | −.0062 | −3 | 24 | 40 | DSGRDYVSQF |
| | | | | | | EGSALGK |
| 1815.8508 | 1815.8446 | −.0062 | −3 | 24 | 40 | DSGRDYVSQF |
| | | | | | | EGSALGK |

| | |
|---|---|
| unnamed protein product [Homo sapiens] | gi\|215415652 |

Peptide Information

| | Obsrv. | | | | End | |
|---|---|---|---|---|---|---|
| Calc. Mass | Mass | ±da | ±ppm | Start Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.6273 | 1283.6271 | −.0002 | 0 | 150 | 160 | DCARAHVDALR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1311.5555 | 1311.5947 | .0392 | 30 | 78 | 88 | ETEGLCQEMSK |
| 1327.5505 | 1327.6265 | .076 | 57 | 78 | 88 | ETEGLCQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLEK |

| | |
|---|---|
| unnamed protein product [Homo sapiens] | gi\|215415640 |

Peptide Information

| | Obsrv. | | | | End | |
|---|---|---|---|---|---|---|
| Calc. Mass | Mass | ±da | ±ppm | Start Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.6273 | 1283.6271 | −.0002 | 0 | 150 | 160 | DCARAHVDALR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDR |
| | | | | | | VK |
| 1467.7914 | 1467.7849 | −.0065 | −4 | 119 | 131 | VEPLRAELQEG |
| | | | | | | AR |
| 1467.7914 | 1467.7849 | −.0065 | −4 | 119 | 131 | VEPLRAELQEG |
| | | | | | | AR |
| 1585.8081 | 1585.8174 | .0093 | 6 | 161 | 173 | THLAPYSDELR |
| | | | | | | QR |
| 1585.8081 | 1585.8174 | .0093 | 6 | 161 | 173 | THLAPYSDELR |
| | | | | | | QR |
| 1612.7854 | 1612.8048 | .0194 | 12 | 46 | 59 | LLDNWDSVTST |
| | | | | | | FSK |
| 1650.8698 | 1650.8733 | .0035 | 2 | 13 | 27 | DLATVYVDVLK |
| | | | | | | DSGR |
| 1815.8508 | 1815.8446 | −.0062 | −3 | 24 | 40 | DSGRDYVSQF |
| | | | | | | EGSALGK |
| 1815.8508 | 1815.8446 | −.0062 | −3 | 24 | 40 | DSGRDYVSQF |
| | | | | | | EGSALGK |
| 2202.1189 | 2202.0879 | −.031 | −14 | 60 | 77 | LREQLGPVTQE |
| | | | | | | FWDNLEK |

| | |
|---|---|
| unnamed protein product [Homo sapiens] | gi\|215415648 |

Peptide Information

| | Obsrv. | | | Start | | |
|---|---|---|---|---|---|---|
| Calc. Mass | Mass | ±da | ±ppm | Seq. | End Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.6273 | 1283.6271 | −.0002 | 0 | 150 | 160 | DCARAHVDALR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDR |
| | | | | | | VK |
| 1467.7914 | 1467.7849 | −.0065 | −4 | 119 | 131 | VEPLRAELQEG |
| | | | | | | AR |
| 1467.7914 | 1467.7849 | −.0065 | −4 | 119 | 131 | VEPLRAELQEG |
| | | | | | | AR |
| 1585.8081 | 1585.8174 | .0093 | 6 | 161 | 173 | THLAPYSDELR |
| | | | | | | QR |
| 1585.8081 | 1585.8174 | .0093 | 6 | 161 | 173 | THLAPYSDELR |
| | | | | | | QR |
| 1612.7854 | 1612.8048 | .0194 | 12 | 46 | 59 | LLDNWDSVTST |
| | | | | | | FSK |
| 1650.8698 | 1650.8733 | .0035 | 2 | 13 | 27 | DLATVYVDVLK |
| | | | | | | DSGR |
| 1815.8508 | 1815.8446 | −.0062 | −3 | 24 | 40 | DSGRDYVSQF |
| | | | | | | EGSALGK |
| 1815.8508 | 1815.8446 | −.0062 | −3 | 24 | 40 | DSGRDYVSQF |
| | | | | | | EGSALGK |

| | |
|---|---|
| unnamed protein product [Homo sapiens] | gi\|215415654 |

Peptide Information

| | Obsrv. | | | Start | End | |
|---|---|---|---|---|---|---|
| Calc. Mass | Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| | 1283.6271 | | | | | |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1311.5555 | 1311.5947 | .0392 | 30 | 78 | 88 | ETEGLCQEMSK |
| 1318.642 | 1318.6689 | .0269 | 20 | 141 | 151 | LSPLGEEMRDR |
| 1327.5505 | 1327.6265 | .076 | 57 | 78 | 88 | ETEGLCQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDSGR |
| 1815.8508 | 1815.8446 | −.0062 | −3 | 24 | 40 | DSGRDYVSQFEGSALGK |
| 1815.8508 | 1815.8446 | −.0062 | −3 | 24 | 40 | DSGRDYVSQFEGSALGK |
| 2202.1189 | 2202.0879 | −.031 | −14 | 60 | 77 | LREQLGPVTQEFWDNLEK |

| | |
|---|---|
| unnamed protein product [Homo sapiens] | gi\|215415628 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDSGR |
| 1815.8508 | 1815.8446 | −.0062 | −3 | 24 | 40 | DSGRDYVSQFEGSALGK |
| 1815.8508 | 1815.8446 | −.0062 | −3 | 24 | 40 | DSGRDYVSQFEGSALGK |
| 2202.1189 | 2202.0879 | −.031 | −14 | 60 | 77 | LREQLGPVTQEFWDNLE K |

| | |
|---|---|
| unnamed protein product [Homo sapiens] | gi\|215415634 |

Peptide Information

| | Obsrv. | | | Start | | |
|---|---|---|---|---|---|---|
| Calc. Mass | Mass | ±da | ±ppm | Seq. | End Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1467.7914 | 1467.7849 | −.0065 | −4 | 119 | 131 | VEPLRAELQEG |
| | | | | | | AR |
| 1467.7914 | 1467.7849 | −.0065 | −4 | 119 | 131 | VEPLRAELQEG |
| | | | | | | AR |
| | 1467.7849 | | | | | |
| 1585.8081 | 1585.8174 | .0093 | 6 | 161 | 173 | THLAPYSDELR |
| | | | | | | QR |
| 1612.7854 | 1612.8048 | .0194 | 12 | 46 | 59 | LLDNWDSVTST |
| | | | | | | FSK |
| 1650.8698 | 1650.8733 | .0035 | 2 | 13 | 27 | DLATVYVDVLK |
| | | | | | | DSGR |
| 1815.8508 | 1815.8446 | −.0062 | −3 | 24 | 40 | DSGRDYVSQF |
| | | | | | | EGSALGK |
| 1815.8508 | 1815.8446 | −.0062 | −3 | 24 | 40 | DSGRDYVSQF |
| | | | | | | EGSALGK |
| 2202.1189 | 2202.0879 | −.031 | −14 | 60 | 77 | LREQLGPVTQE |
| | | | | | | FWDNLEK |

| | |
|---|---|
| unnamed protein product [Homo sapiens] | gi\|215415650 |

Peptide Information

| | | | | Start | | |
|---|---|---|---|---|---|---|
| Calc. Mass | Obsrv. Mass | ±da | ±ppm | Seq. | End Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1311.5555 | 1311.5947 | .0392 | 30 | 78 | 88 | ETEGLCQEMSK |
| 1318.642 | 1318.6689 | .0269 | 20 | 141 | 151 | LSPLGEEMRDR |
| 1327.5505 | 1327.6265 | .076 | 57 | 78 | 88 | ETEGLCQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPW |
| | | | | | | DRVK |
| 1467.7914 | 1467.7849 | −.0065 | −4 | 119 | 131 | VEPLRAELQ |
| | | | | | | EGAR |
| 1467.7914 | 1467.7849 | −.0065 | −4 | 119 | 131 | VEPLRAELQ |
| | | | | | | EGAR |
| 1612.7854 | 1612.8048 | .0194 | 12 | 46 | 59 | LLDNWDSVT |
| | | | | | | STFSK |
| 1650.8698 | 1650.8733 | .0035 | 2 | 13 | 27 | DLATVYVDV |
| | | | | | | LKDSGR |
| 1815.8508 | 1815.8446 | −.0062 | −3 | 24 | 40 | DSGRDYVSQF |
| | | | | | | EGSALGK |
| 1815.8508 | 1815.8446 | −.0062 | −3 | 24 | 40 | DSGRDYVSQF |
| | | | | | | EGSALGK |
| 2202.1189 | 2202.0879 | −.031 | −14 | 60 | 77 | LREQLGPVTQE |
| | | | | | | FWDNLEK |

| | |
|---|---|
| unnamed protein product [Homo sapiens] | gi\|215415626 |

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | 40 | 189 | 195 | LAEYHAK |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDSGR |
| 1815.8508 | 1815.8446 | −.0062 | −3 | 24 | 40 | DSGRDYVSQFEGSALGK |
| 1815.8508 | 1815.8446 | −.0062 | −3 | 24 | 40 | DSGRDYVSQFEGSALGK |
| 2202.1189 | 2202.0879 | −.031 | −14 | 60 | 77 | LREQLGPVTQEFWDNLEK |

| | |
|---|---|
| proapolipoprotein [Homo sapiens] | gi\|178775 |

Peptide Information

| | Obsrv. | | | Start | | |
|---|---|---|---|---|---|---|
| Calc. Mass | Mass | ±da | ±ppm | Seq. | End Seq. | Sequence |
| 831.4359 | 831.4695 | .0336 | 40 | 195 | 201 | LAEYHAK |
| 869.5203 | 869.5491 | .0288 | 33 | 123 | 129 | QKVEPLR |
| 873.4424 | 873.4709 | .0285 | 33 | 130 | 137 | AELQEGAR |
| 1008.5697 | 1008.5928 | .0231 | 23 | 158 | 166 | ARAHVDALR |
| 1012.5786 | 1012.6014 | .0228 | 23 | 213 | 221 | AKPALEDLR |
| 1012.5786 | 1012.6014 | .0228 | 23 | 213 | 221 | AKPALEDLR |
| 1252.6208 | 1252.6487 | .0279 | 22 | 103 | 112 | VQPYLDDFQK |
| 1283.5725 | 1283.6271 | .0546 | 43 | 114 | 122 | WQEEMELYR |
| 1301.6484 | 1301.66 | .0116 | 9 | 167 | 177 | THLAPYSDELR |
| 1301.6484 | 1301.66 | .0116 | 9 | 167 | 177 | THLAPYSDELR |
| 1318.642 | 1318.6689 | .0269 | 20 | 147 | 157 | LSPLGEEMRDR |
| 1323.621 | 1323.6156 | −.0054 | −4 | 84 | 94 | ETEGLRQEMSK |
| 1380.7158 | 1380.7303 | .0145 | 11 | 103 | 113 | VQPYLDDFQKK |
| 1386.7152 | 1386.7365 | .0213 | 15 | 233 | 244 | VSFLSALEEYTK |
| 1400.6693 | 1400.715 | .0457 | 33 | 34 | 46 | DYVSQFEGSAL |
| | | | | | | GK |
| 1411.6675 | 1411.6667 | −.0008 | −1 | 113 | 122 | KWQEEMELYR |
| 1467.7914 | 1467.7849 | −.0065 | −4 | 125 | 137 | VEPLRAELQEG |
| | | | | | | AR |
| 1467.7914 | 1467.7849 | −.0065 | −4 | 125 | 137 | VEPLRAELQEG |
| | | | | | | AR |
| 1585.8081 | 1585.8174 | .0093 | 6 | 167 | 179 | THLAPYSDELR |
| | | | | | | QR |
| 1585.8081 | 1585.8174 | .0093 | 6 | 167 | 179 | THLAPYSDELR |
| | | | | | | QR |
| 1612.7854 | 1612.8048 | .0194 | 12 | 52 | 65 | LLDNWDSVTST |
| | | | | | | FSK |
| 1650.8698 | 1650.8733 | .0035 | 2 | 19 | 33 | DLATVYVDVLK |
| | | | | | | DSGR |
| 1815.8508 | 1815.8446 | −.0062 | −3 | 30 | 46 | DSGRDYVSQF |
| | | | | | | EGSALGK |
| 1815.8508 | 1815.8446 | −.0062 | −3 | 30 | 46 | DSGRDYVSQF |
| | | | | | | EGSALGK |
| 2202.1189 | 2202.0879 | −.031 | −14 | 66 | 83 | LREQLGPVTQE |
| | | | | | | FWDNLEK |

| | |
|---|---|
| unnamed protein product [Homo sapiens] | gi\|215415624 |

Peptide Information

| | | | | Start | | |
|---|---|---|---|---|---|---|
| Calc. Mass | Obsrv. Mass | ±da | ±ppm | Seq. | End Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSP |
| | | | | | | WDR |
| 1226.5437 | 1226.5634 | .0197 | 16 | 1 | 10 | DEPPQSP |
| | | | | | | WDR |
| 1252.6208 | 1252.6487 | .0279 | 22 | 97 | 106 | VQPYLDD |
| | | | | | | FQK |
| 1283.5725 | 1283.6271 | .0546 | 43 | 108 | 116 | WQEEME |
| | | | | | | LYR |
| 1301.6484 | 1301.66 | .0116 | 9 | 161 | 171 | THLAPYS |
| | | | | | | DELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDR |
| | | | | | | VK |
| 1467.7914 | 1467.7849 | −.0065 | −4 | 119 | 131 | VEPLRAELQEG |
| | | | | | | AR |
| 1467.7914 | 1467.7849 | −.0065 | −4 | 119 | 131 | VEPLRAELQEG |
| | | | | | | AR |
| 1612.7854 | 1612.8048 | .0194 | 12 | 46 | 59 | LLDNWDSVTST |
| | | | | | | FSK |
| 1650.8698 | 1650.8733 | .0035 | 2 | 13 | 27 | DLATVYVDVLK |
| | | | | | | DSGR |
| 1815.8508 | 1815.8446 | −.0062 | −3 | 24 | 40 | DSGRDYVSQF |
| | | | | | | EGSALGK |
| 1815.8508 | 1815.8446 | −.0062 | −3 | 24 | 40 | DSGRDYVSQF |
| | | | | | | EGSALGK |
| 2202.1189 | 2202.0879 | −.031 | −14 | 60 | 77 | LREQLGPVTQE |
| | | | | | | FWDNLE K |

| | |
|---|---|
| unnamed protein product [Homo sapiens] | gi\|215415646 |

Peptide Information

| | Obsrv. | | | Start | End | |
|---|---|---|---|---|---|---|
| Calc. Mass | Mass | ±da | ±ppm | Seq. | Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1226.5437 | 1226.5634 | .0197 | 16 | 1 | 10 | DEPPQSPWDR |
| 1226.5437 | 1226.5634 | .0197 | 16 | 1 | 10 | DEPPQSPWDR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| | 1323.6156 | | | | | |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDRVK |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTFSK |
| 1650.8698 | 1650.8733 | .0035 | 2 | 13 | 27 | DLATVYVDVLKDSGR |
| 1815.8508 | 1815.84 | −.0062 | — | 2 | 40 | DSGRDYVSQF |
| 1815.8508 | 1815.84 | −.0062 | — | 2 | 40 | DSGRDYVSQF |

| | |
|---|---|
| apolipoprotein A-I preproprotein [Homo sapiens] | gi\|4557321 |

Protein Group

| | |
|---|---|
| ApoA-I [Homo sapiens] | gi\|296729 |
| Apolipoprotein A-I [Homo sapiens] | gi\|82571472 |
| Apolipoprotein A-I [Homo sapiens] | gi\|13529242 |
| RecName: Full = Apolipoprotein A-I; Short = Apo-AI; | gi\|113992 |
| Short = ApoA-I; AltName: Full = Apolipoprotein A1; | |
| Contains: RecName: | |
| Full = Apolipoprotein A-I(1-242); Flags: | |
| Precursor | |
| apolipoprotein A-I [Homo sapiens] | gi\|37499465 |
| apolipoprotein A-I [Homo sapiens] | gi\|45593391 |
| apolipoprotein A-I precursor variant 1 [Homo sapiens] | gi\|167887482 |
| apolipoprotein A-I precursor variant 2 [Homo sapiens] | gi\|167887483 |
| apolipoprotein A-I precursor variant 3 [Homo sapiens] | gi\|167887484 |
| apolipoprotein A-I precursor variant 4 [Homo sapiens] | gi\|167887485 |
| apolipoprotein A-I precursor variant 5 [Homo sapiens] | gi\|167887486 |
| apolipoprotein A-I, isoform CRA_a [Homo sapiens] | gi\|119587680 |
| apolipoprotein A-I, isoform CRA_a [Homo sapiens] | gi\|119587679 |
| apolipoprotein A-I, isoform CRA_a [Homo sapiens] | gi\|119587678 |
| apolipoprotein AI precursor [Homo sapiens] | gi\|296635 |
| apolipoprotein AI | |
| precursor [Homo | |
| sapiens] | |
| preproapolipoprotein A-I [Homo sapiens] | gi\|178768 |
| preproapolipoprotein AI [Homo sapiens] | gi\|28772 |
| preproapolipoprotein [Homo sapiens] | gi\|512410 |
| proapolipoprotein [Homo sapiens] | gi\|178773 |
| unnamed protein product [Homo sapiens] | gi\|732753 |
| unnamed protein product [Homo sapiens] | gi\|158257894 |

Peptide Information

| | | | | Start | | |
|---|---|---|---|---|---|---|
| Calc. Mass | Obsrv. Mass | ±da | ±ppm | Seq. | End Seq. | Sequence |
| 831.4359 | 831.4695 | .0336 | 40 | 213 | 219 | LAEYHAK |
| 869.5203 | 869.5491 | .0288 | 33 | 141 | 147 | QKVEPLR |
| 873.4424 | 873.4709 | .0285 | 33 | 148 | 155 | AELQEGAR |
| 1008.5697 | 1008.5928 | .0231 | 23 | 176 | 184 | ARAHVDALR |
| 1012.5786 | 1012.6014 | .0228 | 23 | 231 | 239 | AKPALEDLR |
| 1012.5786 | 1012.6014 | .0228 | 23 | 231 | 239 | AKPALEDLR |
| 1252.6208 | 1252.6487 | .0279 | 22 | 121 | 130 | VQPYLDDFQK |
| 1283.5725 | 1283.6271 | .0546 | 43 | 132 | 140 | WQEEMELYR |
| 1301.6484 | 1301.66 | .0116 | 9 | 185 | 195 | THLAPYSDELR |
| 1301.6484 | 1301.66 | .0116 | 9 | 185 | 195 | THLAPYSDELR |
| 1318.642 | 1318.6689 | .0269 | 20 | 165 | 175 | LSPLGEEMRDR |
| 1323.621 | 1323.6156 | −.0054 | −4 | 102 | 112 | ETEGLRQEMSK |
| 1380.7158 | 1380.7303 | .0145 | 11 | 121 | 131 | VQPYLDDFQKK |
| 1386.7152 | 1386.7365 | .0213 | 15 | 251 | 262 | VSFLSALEEYTK |
| 1400.6693 | 1400.715 | .0457 | 33 | 52 | 64 | DYVSQFEGSAL |
| | | | | | | GK |
| 1411.6675 | 1411.6667 | −.0008 | −1 | 131 | 140 | KWQEEMELYR |
| 1467.7914 | 1467.7849 | −.0065 | −4 | 143 | 155 | VEPLRAELQEG |
| | | | | | | AR |
| 1467.7914 | 1467.7849 | −.0065 | −4 | 143 | 155 | VEPLRAELQEG |
| | | | | | | AR |
| 1585.8081 | 1585.8174 | .0093 | 6 | 185 | 197 | THLAPYSDELR |
| | | | | | | QR |
| 1585.8081 | 1585.8174 | .0093 | 6 | 185 | 197 | THLAPYSDELR |
| | | | | | | QR |
| 1612.7854 | 1612.8048 | .0194 | 12 | 70 | 83 | LLDNWDSVTST |
| | | | | | | FSK |
| 1650.8698 | 1650.8733 | .0035 | 2 | 37 | 51 | DLATVYVDVLK |
| | | | | | | DSGR |
| 1815.8508 | 1815.8446 | −.0062 | −3 | 48 | 64 | DSGRDYVSQF |
| | | | | | | EGSALGK |
| 1815.8508 | 1815.8446 | −.0062 | −3 | 48 | 64 | DSGRDYVSQF |
| | | | | | | EGSALGK |
| 2202.1189 | 2202.0879 | −.031 | −14 | 84 | 101 | LREQLGPVTQE |
| | | | | | | FWDNLEK |

| | |
|---|---|
| unnamed protein product [Homo sapiens] | gi\|215415644 |

Peptide Information

| | | | | Start | End | |
|---|---|---|---|---|---|---|
| Calc. Mass | Obsrv. Mass | ±da | ±ppm | Seq. | Seq. | Szequence |
| 831.4359 | 831.4695 | .0336 | .0 | 89 | 95 | [symbol] AEYHAK |
| 869.5203 | 869.5491 | .0288 | [symbol] 3 | 17 | 23 | [symbol] KVEPLR |
| 873.4424 | 873.4709 | .0285 | [symbol] 3 | 24 | 31 | [symbol] ELQEGAR |
| 1008.5697 | 1008.5928 | .0231 | [symbol] 3 | 52 | 60 | [symbol] RAHVDALR |
| 1012.5786 | 1012.6014 | .0228 | [symbol] 3 | [symbol] 07 | [symbol] 15 | [symbol] KPALEDLR |
| 1012.5786 | 1012.6014 | .0228 | [symbol] 3 | [symbol] 07 | [symbol] 15 | [symbol] KPALEDLR |
| 1226.5437 | 1226.5634 | .0197 | 6 | | 0 | [symbol] EPPQSPWDR |
| 1226.5437 | 1226.5634 | .0197 | 6 | | 0 | [symbol] EPPQSPWDR |
| 1252.6208 | 1252.6487 | .0279 | [symbol] 2 | [symbol] 7 | 06 | [symbol] QPYLDDFQK |
| 1283.5725 | 1283.6271 | .0546 | [symbol] 3 | 08 | 16 | VQEEMELYR |
| 1301.6484 | 1301.66 | .0116 | [symbol] | 61 | 71 | ⁻HLAPYSDELR |
| 1301.6484 | 1301.66 | .0116 | [symbol] | 61 | 71 | ⁻HLAPYSDELR |
| 1318.642 | 1318.6689 | .0269 | [symbol] 0 | 41 | 51 | [symbol] SPLGEEMRDR |
| 1323.621 | 1323.6156 | −.0054 | 4 | [symbol] 8 | [symbol] 8 | [symbol] TEGLRQEMSK |
| 1380.7158 | 1380.7303 | .0145 | 1 | [symbol] 7 | 07 | [symbol] QPYLDDFQKK |
| 1386.7152 | 1386.7365 | .0213 | 5 | [symbol] 27 | [symbol] 38 | [symbol] SFLSALEEYTK |
| 1400.6693 | 1400.715 | .0457 | [symbol] 3 | [symbol] 8 | [symbol] 0 | [symbol] YVSQFEGSALGK |
| 1411.6675 | 1411.6667 | −.0008 | 1 | 07 | 16 | [symbol] WQEEMELYR |
| 1453.707 | 1453.7174 | .0104 | [symbol] | | 2 | [symbol] EPPQSPWDRVK |
| 1467.7914 | 1467.7849 | −.0065 | 4 | 19 | 31 | [symbol] EPLRAELQEGAR |
| 1467.7914 | 1467.7849 | −.0065 | 4 | 19 | 31 | [symbol] EPLRAELQEGAR |
| 1612.7854 | 1612.8048 | .0194 | 2 | [symbol] 6 | [symbol] 9 | [symbol] LDNWDSVTSTFSK |
| 1650.8698 | 1650.8733 | .0035 | [symbol] | 3 | [symbol] 7 | [symbol] LATVYVDVLKDSGR |
| 1815.8508 | 1815.8446 | −.0062 | 3 | [symbol] 4 | [symbol] 0 | [symbol] SGRDYVSQFEGS |
| | | | | | | ALGK |
| 1815.8508 | 1815.8446 | −.0062 | 3 | [symbol] 4 | .0 | [symbol] SGRDYVSQFEGS |
| | | | | | | ALGK |

| | |
|---|---|
| unnamed protein product [Homo sapiens] | gi\|215415658 |

[symbol]

indicates data missing or illegible when filed

Peptide Information

| | | | | Start | End | |
|---|---|---|---|---|---|---|
| Calc. Mass | Obsrv. Mass | ±da | ±ppm | Seq. | Seq. | Sequence |
| 831.4359 | 831.4695 | .0336 | [symbol] 0 | 89 | 95 | [symbol] AEYHAK |
| 869.5203 | 869.5491 | .0288 | [symbol] 3 | 17 | 23 | [symbol] KVEPLR |
| 873.4424 | 873.4709 | .0285 | [symbol] 3 | 24 | 31 | [symbol] ELQEGAR |
| 1226.5437 | 1226.5634 | .0197 | 6 | | 0 | [symbol] EPPQSPWDR |
| 1226.5437 | 1226.5634 | .0197 | 6 | | 0 | [symbol] EPPQSPWDR |
| | 1226.5634 | | | | | |
| 1252.6208 | 1252.6487 | .0279 | [symbol] 2 | [symbol] 7 | 06 | [symbol] QPYLDDFQK |
| 1283.6273 | 1283.6271 | −.0002 | [symbol] | 50 | 60 | [symbol] CARAHVDALR |
| 1301.6484 | 1301.66 | .0116 | [symbol] | 61 | 71 | ⁻HLAPYSDELR |
| 1301.6484 | 1301.66 | .0116 | [symbol] | 61 | 71 | ⁻HLAPYSDELR |
| 1323.621 | 1323.6156 | −.0054 | 4 | [symbol] 8 | [symbol] 8 | [symbol] TEGLRQEMSK |
| 1380.7158 | 1380.7303 | .0145 | 1 | [symbol] 7 | 07 | [symbol] QPYLDDFQKK |
| 1386.7152 | 1386.7365 | .0213 | 5 | [symbol] 27 | [symbol] 38 | [symbol] SFLSALEEYTK |
| 1400.6693 | 1400.715 | .0457 | [symbol] 3 | [symbol] 8 | [symbol] 0 | [symbol] YVSQFEGSALGK |
| 1411.6675 | 1411.6667 | −.0008 | 1 | 07 | 16 | [symbol] WQEEMELYR |
| 1453.707 | 1453.7174 | .0104 | [symbol] | | 2 | [symbol] EPPQSPWDRVK |
| 1467.7914 | 1467.7849 | −.0065 | 4 | 19 | 31 | [symbol] EPLRAELQEGAR |
| 1467.7914 | 1467.7849 | −.0065 | 4 | 19 | 31 | [symbol] EPLRAELQEGAR |
| 1585.8081 | 1585.8174 | .0093 | [symbol] | 61 | 73 | ⁻HLAPYSDELRQR |
| 1585.8081 | 1585.8174 | .0093 | [symbol] | 61 | 73 | ⁻HLAPYSDELRQR |
| 1612.7854 | 1612.8048 | .0194 | 2 | [symbol] 6 | [symbol] 9 | [symbol] LDNWDSVTSTFSK |
| 1650.8698 | 1650.8733 | .0035 | [symbol] | 3 | [symbol] 7 | [symbol] LATVYVDVLKDSGR |
| 1815.8508 | 1815.8446 | −.0062 | 3 | [symbol] 4 | [symbol] 0 | [symbol] SGRDYVSQFEGS |
| | | | | | | ALGK |
| 1815.8508 | 1815.8446 | −.0062 | 3 | [symbol] 4 | [symbol] 0 | [symbol] SGRDYVSQFEGS |
| | | | | | | ALGK |
| 2202.1189 | 2202.0879 | −.031 | 14 | [symbol] 0 | [symbol] 7 | [symbol] REQLGPVTQEFW |
| | | | | | | DNLE [symbol] |

| | |
|---|---|
| unnamed protein product [Homo sapiens] | gi\|215415656 |

[symbol]

indicates data missing or illegible when filed

Peptide Information

| | | | | Start | | |
|---|---|---|---|---|---|---|
| Calc. Mass | Obsrv. Mass | ±da | ±ppm | Seq. | End Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1012.5786 | 1012.6014 | .0228 | 23 | 207 | 215 | AKPALEDLR |
| 1012.5786 | 1012.6014 | .0228 | 23 | 207 | 215 | AKPALEDLR |
| 1226.5437 | 1226.5634 | .0197 | 16 | 1 | 10 | DEPPQSPWDR |
| 1226.5437 | 1226.5634 | .0197 | 16 | 1 | 10 | DEPPQSP |
| | | | | | | WDR |
| 1252.6208 | 1252.6487 | .0279 | 22 | 97 | 106 | VQPYLDD |
| | | | | | | FQK |
| 1283.6273 | 1283.6271 | −.0002 | 0 | 150 | 160 | DCARAHV |
| | | | | | | DALR |
| 1301.6484 | 1301.66 | .0116 | 9 | 161 | 171 | THLAPYS |
| | | | | | | DELR |
| 1301.6484 | 1301.66 | .0116 | 9 | 161 | 171 | THLAPYS |
| | | | | | | DELR |
| 1323.621 | 1323.6156 | −.0054 | −4 | 78 | 88 | ETEGLRQ |
| | | | | | | EMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSAL |
| | | | | | | GK |
| 1411.6675 | 1411.6667 | −.0008 | −1 | 107 | 116 | KWQEEMELYR |
| 1453.707 | 1453.7174 | .0104 | 7 | 1 | 12 | DEPPQSPWDR |
| | | | | | | VK |
| 1467.7914 | 1467.7849 | −.0065 | −4 | 119 | 131 | VEPLRAELQEG |
| | | | | | | AR |
| 1467.7914 | 1467.7849 | −.0065 | −4 | 119 | 131 | VEPLRAELQEG |
| | | | | | | AR |
| 1612.7854 | 1612.8048 | .0194 | 12 | 46 | 59 | LLDNWDSVTST |
| | | | | | | FSK |
| 1650.8698 | 1650.8733 | .0035 | 2 | 13 | 27 | DLATVYVDVLK |
| | | | | | | DSGR |
| 1815.8508 | 1815.8446 | −.0062 | −3 | 24 | 40 | DSGRDYVSQF |
| | | | | | | EGSALGK |
| 1815.8508 | 1815.8446 | −.0062 | −3 | 24 | 40 | DSGRDYVSQF |
| | | | | | | EGSALGK |
| 2202.1189 | 2202.0879 | −.031 | −14 | 60 | 77 | LREQLGPVTQE |
| | | | | | | FWDNLEK |

| | |
|---|---|
| proapo-A-I protein [Homo sapiens] | gi\|178777 |

Peptide Information

| | | | | Start | | |
|---|---|---|---|---|---|---|
| Calc. Mass | Obsrv. Mass | ±da | ±ppm | Seq. | End Seq. | Sequence |
| 831.4359 | 831.4695 | .0336 | 40 | 213 | 219 | LAEYHAK |
| 873.4424 | 873.4709 | .0285 | 33 | 148 | 155 | AELQEGAR |
| 1008.5697 | 1008.5928 | .0231 | 23 | 176 | 184 | ARAHVDALR |
| 1012.5786 | 1012.6014 | .0228 | 23 | 231 | 239 | AKPALEDLR |
| 1012.5786 | 1012.6014 | .0228 | 23 | 231 | 239 | AKPALEDLR |
| 1252.6208 | 1252.6487 | .0279 | 22 | 121 | 130 | VQPYLDDFQK |
| 1283.5725 | 1283.6271 | .0546 | 43 | 132 | 140 | WQEEMELYR |
| 1301.6484 | 1301.66 | .0116 | 9 | 185 | 195 | THLAPYSDELR |
| 1301.6484 | 1301.66 | .0116 | 9 | 185 | 195 | THLAPYSDELR |
| 1318.642 | 1318.6689 | .0269 | 20 | 165 | 175 | LSPLGEEMRDR |
| 1323.621 | 1323.6156 | −.0054 | −4 | 102 | 112 | ETEGLRQEMSK |
| 1380.7158 | 1380.7303 | .0145 | 11 | 121 | 131 | VQPYLDDFQKK |
| 1386.7152 | 1386.7365 | .0213 | 15 | 251 | 262 | VSFLSALEEYTK |
| 1400.6693 | 1400.715 | .0457 | 33 | 52 | 64 | DYVSQFEGSALGK |
| 1411.6675 | 1411.6667 | −.0008 | −1 | 131 | 140 | KWQEEMELYR |
| 1453.7758 | 1453.7174 | −.0584 | −40 | 143 | 155 | VDPLRAELQEGAR |
| 1585.8081 | 1585.8174 | .0093 | 6 | 185 | 197 | THLAPYSDELRQR |
| 1585.8081 | 1585.8174 | .0093 | 6 | 185 | 197 | THLAPYSDELRQR |
| 1612.7854 | 1612.8048 | .0194 | 12 | 70 | 83 | LLDNWDSVTSTF |
| | | | | | | SK |
| 1650.8698 | 1650.8733 | .0035 | 2 | 37 | 51 | DLATVYVDVLKD |
| | | | | | | SGR |
| | 1650.8733 | | | | | |
| 1815.8508 | 1815.8446 | −.0062 | −3 | 48 | 64 | DSGRDYVSQF |
| | | | | | | EGSALGK |
| 1815.8508 | 1815.8446 | −.0062 | −3 | 48 | 64 | DSGRDYVSQF |
| | | | | | | EGSALGK |
| 2202.1189 | 2202.0879 | −.031 | −14 | 84 | 101 | LREQLGPVTQE |
| | | | | | | FWDNLE K |

| | |
|---|---|
| unnamed protein product [Homo sapiens] | gi\|215415642 |

Peptide Information

| | | | | Start | | |
|---|---|---|---|---|---|---|
| Calc. Mass | Obsrv. Mass | ±da | ±ppm | Seq. | End Seq. | Sequence |
| 831.4359 | 831.4695 | .0336 | 40 | 189 | 195 | LAEYHAK |
| 869.5203 | 869.5491 | .0288 | 33 | 117 | 123 | QKVEPLR |
| 873.4424 | 873.4709 | .0285 | 33 | 124 | 131 | AELQEGAR |
| 1008.5697 | 1008.5928 | .0231 | 23 | 152 | 160 | ARAHVDALR |
| 1252.6208 | 1252.6487 | .0279 | 22 | 97 | 106 | VQPYLDDFQK |
| 1283.5725 | 1283.6271 | .0546 | 43 | 108 | 116 | WQEEMELYR |
| 1301.6484 | 1301.66 | .0116 | 9 | 161 | 171 | THLAPYSDELR |
| 1301.6484 | 1301.66 | .0116 | 9 | 161 | 171 | THLAPYSDELR |
| 1318.642 | 1318.6689 | .0269 | 20 | 141 | 151 | LSPLGEEMRDR |
| 1323.621 | 1323.6156 | −.0054 | −4 | 78 | 88 | ETEGLRQEMSK |
| 1380.7158 | 1380.7303 | .0145 | 11 | 97 | 107 | VQPYLDDFQKK |
| 1386.7152 | 1386.7365 | .0213 | 15 | 227 | 238 | VSFLSALEEYTK |
| 1400.6693 | 1400.715 | .0457 | 33 | 28 | 40 | DYVSQFEGSALGK |
| 1411.6675 | 1411.6667 | −.0008 | −1 | 107 | 116 | KWQEEMELYR |
| 1467.7914 | 1467.7849 | −.0065 | −4 | 119 | 131 | VEPLRAELQEGAR |
| 1467.7914 | 1467.7849 | −.0065 | −4 | 119 | 131 | VEPLRAELQEGAR |
| 1585.8081 | 1585.8174 | .0093 | 6 | 161 | 173 | THLAPYSDELRQR |
| 1585.8081 | 1585.8174 | .0093 | 6 | 161 | 173 | THLAPYSDELRQR |
| 1612.7854 | 1612.8048 | .0194 | 12 | 46 | 59 | LLDNWDSVTSTF |
| | | | | | | SK |
| 1650.8698 | 1650.8733 | .0035 | 2 | 13 | 27 | DLATVYVDVLKD |
| | | | | | | SGR |
| 1815.8508 | 1815.8446 | −.0062 | −3 | 24 | 40 | DSGRDYVSQFE |
| | | | | | | GSALGK |
| 1815.8508 | 1815.8446 | −.0062 | −3 | 24 | 40 | DSGRDYVSQFE |
| | | | | | | GSALGK |
| 2202.1189 | 2202.0879 | −.031 | −14 | 60 | 77 | LREQLGPVTQEF |
| | | | | | | WDNLE |

Peptide Information

| | | | |
|---|---|---|---|
| | 63/C12 | | Instr./Gel |
| | | | Origin |
| | [1] Sample Project | | Instrument |
| | 20110503 | | Sample |
| | | | Name |
| Protein Name- | KH12 (SEQ ID NO. KH12) | Accession No. | |

| | |
|---|---|
| unnamed protein product [Homo sapiens] | gi\|189066554 |

Peptide Information

| | Obsrv. | | | Start | | |
|---|---|---|---|---|---|---|
| Calc. Mass | Mass | ±da | ±ppm | Seq. | End Seq. | Sequence |
| 836.4009 | 836.415 | .0141 | 17 | 218 | 224 | GQQYQGR |
| 836.4009 | 836.415 | .0141 | 17 | 218 | 224 | GQQYQGR |
| 874.4781 | 874.4888 | .0107 | 12 | 501 | 508 | VTGWGNLK |
| 951.4894 | 951.5774 | .088 | 92 | 434 | 440 | YERNIEK |
| 971.425 | 971.4321 | .0071 | 7 | 87 | 94 | YTACETAR |
| 1148.5518 | 1148.5477 | −.0041 | −4 | 125 | 133 | SGIECQLWR |
| 1148.5518 | 1148.5477 | −.0041 | −4 | 125 | 133 | SGIECQLWR |
| 1189.579 | 1189.5732 | −.0058 | −5 | 600 | 608 | YGFYTHVFR |
| 1194.6001 | 1194.5807 | −.0194 | −16 | 354 | 363 | ELLESYIDGR |
| 1194.6001 | 1194.5807 | −.0194 | −16 | 354 | 363 | ELLESYIDGR |
| 1265.6022 | 1265.592 | −.0102 | −8 | 453 | 461 | YNWRENLDR |
| 1489.7223 | 1489.7014 | −.0209 | −14 | 597 | 608 | DGKYGFYTHVFR |
| 1542.62 | 1542.6259 | .0059 | 4 | 561 | 575 | GDACEGDSGG |
| | | | | | | PFVMK |
| 1561.7281 | 1561.6975 | −.0306 | −20 | 315 | 327 | TATSEYQTFFN |
| | | | | | | PR |
| 1561.7281 | 1561.6975 | −.0306 | −20 | 315 | 327 | TATSEYQTFFN |
| | | | | | | PR |
| 1645.8214 | 1645.7874 | −.034 | −21 | 385 | 399 | SPQELLCGASL |
| | | | | | | ISDR |
| 1698.7211 | 1698.6948 | −.0263 | −15 | 560 | 575 | RGDACEGDSG |
| | | | | | | GPFVMK |
| 1773.9164 | 1773.8727 | −.0437 | −25 | 384 | 399 | KSPQELLCGA |
| | | | | | | SLISDR |
| 1883.8956 | 1883.8428 | −.0528 | −28 | 328 | 344 | TFGSGEADCG |
| | | | | | | LRPLFEK |
| 1883.8956 | 1883.8428 | −.0528 | −28 | 328 | 344 | TFGSGEADCG |
| | | | | | | LRPLFEK |
| 1963.908 | 1963.848 | −.06 | −31 | 248 | 263 | HQDFNSAVQL |
| | | | | | | VENFCR |
| 1968.8903 | 1968.8427 | −.0476 | −24 | 100 | 117 | LAACLEGNCA |
| | | | | | | EGLGTNYR |
| 2001.9158 | 2001.8715 | −.0443 | −22 | 544 | 560 | ITDNMFCAGYK |
| | | | | | | PDEGKR |
| 2011.9906 | 2011.9392 | −.0514 | −26 | 328 | 345 | TFGSGEADCGL |
| | | | | | | RPLFEK K |
| 2017.9106 | 2017.8978 | −.0128 | −6 | 544 | 560 | ITDNMFCAGYK |
| | | | | | | PDEGKR |
| 2070.9761 | 2070.9165 | −.0596 | −29 | 199 | 217 | SEGSSVNLSPP |
| | | | | | | LEQCVP |
| | | | | | | DR |
| 2200.9275 | 2200.9404 | .0129 | 6 | 159 | 177 | NPDSSTMGPW |
| | | | | | | CYTTDPT |
| | | | | | | VR |
| 2212.0122 | 2212.0205 | .0083 | 4 | 98 | 117 | DKLAACLEGNC |
| | | | | | | AEGLGT |
| | | | | | | NYR |
| 2212.0122 | 2212.0205 | .0083 | 4 | 98 | 117 | DKLAACLEGNC |
| | | | | | | AEGLGT |
| | | | | | | NYR |
| 2232.2534 | 2232.1794 | −.074 | −33 | 518 | 537 | GQPSVLQVVNL |
| | | | | | | PIVERPV |
| | | | | | | CK |
| 2232.2534 | 2232.1794 | −.074 | −33 | 518 | 537 | GQPSVLQVVNL |
| | | | | | | PIVERPV |
| | | | | | | CK |
| 2281.0991 | 2281.072 | −.0271 | −12 | 364 | 383 | IVEGSDAEIGM |
| | | | | | | SPWQVM |
| | | | | | | LFR |
| 2292.0417 | 2292.0005 | −.0412 | −18 | 179 | 198 | QECSIPVCGQD |
| | | | | | | QVTVAM |
| | | | | | | TPR |
| 2448.1428 | 2448.1128 | −.03 | −12 | 178 | 198 | RQECSIPVCGQ |
| | | | | | | DQVTVA MTPR |
| 2888.3591 | 2888.29 | −.0691 | −24 | 199 | 224 | SEGSSVNLSP |
| | | | | | | PLEQCVP |
| | | | | | | DRGQQYQGR |
| 2888.3591 | 2888.29 | −.0691 | −24 | 199 | 224 | SEGSSVNLSP |
| | | | | | | PLEQCVP |
| | | | | | | DRGQQYQGR |

| | |
|---|---|
| unnamed protein product [Homo sapiens] | gi\|194383496 |

Protein Group

| | Obsrv. | | | Start | End | |
|---|---|---|---|---|---|---|
| Calc. Mass | Mass | ±da | ±ppm | Seq. | Seq. | Sequence |
| 836.4009 | 836.415 | .0141 | 17 | 201 | 207 | GQQYQGR |
| 836.4009 | 836.415 | .0141 | 17 | 201 | 207 | GQQYQGR |
| 874.4781 | 874.4888 | .0107 | 12 | 484 | 491 | VTGWGNLK |
| 951.4894 | 951.5774 | .088 | 92 | 417 | 423 | YERNIEK |
| 971.425 | 971.4321 | .0071 | 7 | 70 | 77 | YTACETAR |
| 1148.5518 | 1148.5477 | −.0041 | −4 | 108 | 116 | SGIECQLWR |
| 1148.5518 | 1148.5477 | −.0041 | −4 | 108 | 116 | SGIECQLWR |
| 1189.579 | 1189.5732 | −.0058 | −5 | 583 | 591 | YGFYTHVFR |
| 1194.6001 | 1194.5807 | −.0194 | −16 | 337 | 346 | ELLESYIDGR |
| 1194.6001 | 1194.5807 | −.0194 | −16 | 337 | 346 | ELLESYIDGR |
| 1265.6022 | 1265.592 | −.0102 | −8 | 436 | 444 | YNWRENLDR |
| 1489.7223 | 1489.7014 | −.0209 | −14 | 580 | 591 | DGKYGFYTHVFR |
| 1542.62 | 1542.6259 | .0059 | 4 | 544 | 558 | GDACEGDSGGPF |
| | | | | | | VMK |
| 1561.7281 | 1561.6975 | −.0306 | −20 | 298 | 310 | TATSEYQTFFNPR |
| 1561.7281 | 1561.6975 | −.0306 | −20 | 298 | 310 | TATSEYQTFFNPR |
| 1645.8214 | 1645.7874 | −.034 | −21 | 368 | 382 | SPQELLCGASLISDR |
| 1698.7211 | 1698.6948 | −.0263 | −15 | 543 | 558 | RGDACEGDSG |
| | | | | | | GPFVMK |
| 1773.9164 | 1773.8727 | −.0437 | −25 | 367 | 382 | KSPQELLCGAS |
| | | | | | | LISDR |
| 1883.8956 | 1883.8428 | −.0528 | −28 | 311 | 327 | TFGSGEADCG |
| | | | | | | LRPLFEK |
| 1883.8956 | 1883.8428 | −.0528 | −28 | 311 | 327 | TFGSGEADCG |
| | | | | | | LRPLFEK |
| 1963.908 | 1963.848 | −.06 | −31 | 231 | 246 | HQDFNSAVQL |
| | | | | | | VENFCR |
| 1968.8903 | 1968.8427 | −.0476 | −24 | 83 | 100 | LAACLEGNCAE |
| | | | | | | GLGTNYR |
| 2001.9158 | 2001.8715 | −.0443 | −22 | 527 | 543 | ITDNMFCAGYK |
| | | | | | | PDEGKR |
| 2011.9906 | 2011.9392 | −.0514 | −26 | 311 | 328 | TFGSGEADCG |
| | | | | | | LRPLFEKK |
| 2017.9106 | 2017.8978 | −.0128 | −6 | 527 | 543 | ITDNMFCAGYK |
| | | | | | | PDEGKR |
| 2070.9761 | 2070.9165 | −.0596 | −29 | 182 | 200 | SEGSSVNLSPP |
| | | | | | | LEQCVP |
| | | | | | | DR |
| 2212.0122 | 2212.0205 | .0083 | 4 | 81 | 100 | DKLAACLEGNC |
| | | | | | | AEGLGT |
| | | | | | | NYR |
| 2212.0122 | 2212.0205 | .0083 | 4 | 81 | 100 | DKLAACLEGNC |
| | | | | | | AEGLGT |
| | | | | | | NYR |
| 2232.2534 | 2232.1794 | −.074 | −33 | 501 | 520 | GQPSVLQVVNL |
| | | | | | | PIVERPV |
| | | | | | | CK |
| 2232.2534 | 2232.1794 | −.074 | −33 | 501 | 520 | GQPSVLQVVNL |
| | | | | | | PIVERPV CK |
| 2281.0991 | 2281.072 | −.0271 | −12 | 347 | 366 | IVEGSDAEIGM |
| | | | | | | SPWQVM |
| | | | | | | LFR |
| 2292.0417 | 2292.0005 | −.0412 | −18 | 162 | 181 | QECSIPVCGQD |
| | | | | | | QVTVAM |
| | | | | | | TPR |
| 2448.1428 | 2448.1128 | −.03 | −12 | 161 | 181 | RQECSIPVCGQ |
| | | | | | | DQVTVA |
| | | | | | | MTPR |
| 2888.3591 | 2888.29 | −.0691 | −24 | 182 | 207 | SEGSSVNLSPP |
| | | | | | | LEQCVP |
| | | | | | | DRGQQYQGR |
| 2888.3591 | 2888.29 | −.0691 | −24 | 182 | 207 | SEGSSVNLSPP |
| | | | | | | LEQCVP |
| | | | | | | DRGQQYQGR |

| | |
|---|---|
| unnamed protein product [Homo sapiens] | gi\|1335344 |
| coagulation factor II (thrombin), isoform CRA_a | gi\|119588383 |
| [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 836.4009 | 836.415 | .0141 | 17 | 211 | 217 | GQQYQGR |
| 836.4009 | 836.415 | .0141 | 17 | 211 | 217 | GQQYQGR |
| 874.4781 | 874.4888 | .0107 | 12 | 494 | 501 | VTGWGNLK |
| | 874.4888 | | | | | |
| 951.4894 | 951.5774 | .088 | 92 | 427 | 433 | YERNIEK |
| 971.425 | 971.4321 | .0071 | 7 | 80 | 87 | YTACETAR |
| 1148.5518 | 1148.5477 | −.0041 | −4 | 118 | 126 | SGIECQLWR |
| 1148.5518 | 1148.5477 | −.0041 | −4 | 118 | 126 | SGIECQLWR |
| 1189.579 | 1189.5732 | −.0058 | −5 | 593 | 601 | YGFYTHVFR |
| 1194.6001 | 1194.5807 | −.0194 | −16 | 347 | 356 | ELLESYIDGR |
| 1194.6001 | 1194.5807 | −.0194 | −16 | 347 | 356 | ELLESYIDGR |
| 1265.6022 | 1265.592 | −.0102 | −8 | 446 | 454 | YNWRENLDR |
| 1489.7223 | 1489.7014 | −.0209 | −14 | 590 | 601 | DGKYGFYTHVFR |
| 1542.62 | 1542.6259 | .0059 | 4 | 554 | 568 | GDACEGDSGGPF |
| | | | | | | VMK |
| 1561.7281 | 1561.6975 | −.0306 | −20 | 308 | 320 | TATSEYQTFFNPR |
| 1561.7281 | 1561.6975 | −.0306 | −20 | 308 | 320 | TATSEYQTFFNPR |
| 1645.8214 | 1645.7874 | −.034 | −21 | 378 | 392 | SPQELLCGASLISDR |
| 1698.7211 | 1698.6948 | −.0263 | −15 | 553 | 568 | RGDACEGDSGGP |
| | | | | | | FVMK |
| 1773.9164 | 1773.8727 | −.0437 | −25 | 377 | 392 | KSPQELLCGASLIS |
| | | | | | | DR |
| 1883.8956 | 1883.8428 | −.0528 | −28 | 321 | 337 | TFGSGEADCGLRP |
| | | | | | | LFEK |
| 1883.8956 | 1883.8428 | −.0528 | −28 | 321 | 337 | TFGSGEADCGLRP |
| | | | | | | LFEK |
| 1963.908 | 1963.848 | −.06 | −31 | 241 | 256 | HQDFNSAVQLVEN |
| | | | | | | FCR |
| 1968.8903 | 1968.8427 | −.0476 | −24 | 93 | 110 | LAACLEGNCAEGL |
| | | | | | | GTNYR |
| 2001.9158 | 2001.8715 | −.0443 | −22 | 537 | 553 | ITDNMFCAGYK |
| | | | | | | PDEGKR |
| 2011.9906 | 2011.9392 | −.0514 | −26 | 321 | 338 | TFGSGEADCG |
| | | | | | | LRPLFEKK |
| 2017.9106 | 2017.8978 | −.0128 | −6 | 537 | 553 | ITDNMFCAGYK |
| | | | | | | PDEGKR |
| 2070.9761 | 2070.9165 | −.0596 | −29 | 192 | 210 | SEGSSVNLSPP |
| | | | | | | LEQCVP |
| | | | | | | DR |
| 2212.0122 | 2212.0205 | .0083 | 4 | 91 | 110 | DKLAACLEGNC |
| | | | | | | AEGLGT |
| | | | | | | NYR |
| 2212.0122 | 2212.0205 | .0083 | 4 | 91 | 110 | DKLAACLEGNC |
| | | | | | | AEGLGT |
| | | | | | | NYR |
| 2232.2534 | 2232.1794 | −.074 | −33 | 511 | 530 | GQPSVLQVVNL |
| | | | | | | PIVERPV |
| | | | | | | CK |
| 2232.2534 | 2232.1794 | −.074 | −33 | 511 | 530 | GQPSVLQVVNL |
| | | | | | | PIVERPV |
| | | | | | | CK |
| 2281.0991 | 2281.072 | −.0271 | −12 | 357 | 376 | IVEGSDAEIGM |
| | | | | | | SPWQVM |
| | | | | | | LFR |
| 2292.0417 | 2292.0005 | −.0412 | −18 | 172 | 191 | QECSIPVCGQD |
| | | | | | | QVTVAM |
| | | | | | | TPR |
| 2448.1428 | 2448.1128 | −.03 | −12 | 171 | 191 | RQECSIPVCGQ |
| | | | | | | DQVTVA |
| | | | | | | MTPR |
| 2888.3591 | 2888.29 | −.0691 | −24 | 192 | 217 | SEGSSVNLSPP |
| | | | | | | LEQCVP |
| | | | | | | DRGQQYQGR |
| 2888.3591 | 2888.29 | −.0691 | −24 | 192 | 217 | SEGSSVNLSPP |
| | | | | | | LEQCVP |
| | | | | | | DRGQQYQGR |

| | |
|---|---|
| prothrombin preproprotein [Homo sapiens] | gi\|4503635 |

Protein Group

| | |
|---|---|
| Coagulation factor II (thrombin) [Homo sapiens] | gi\|30802115 |
| RecName: Full = Prothrombin; AltName: | gi\|135807 |
| Full = Coagulation factor II; | |
| Contains: RecName: | |
| Full = Activation peptide fragment 1; | |
| Contains: RecName: | |
| Full = Activation peptide fragment 2; | |
| Contains: RecName: | |
| Full = Thrombin light chain; | |
| Contains: RecName: Full = Thrombin | |
| heavy chain; Flags: | |
| Precursor | |
| coagulation factor II (thrombin) [Homo sapiens] | gi\|18653448 |
| coagulation factor II (thrombin), isoform CRA_c | gi\|119588385 |
| [Homo sapiens] | |
| coagulation factor II precursor variant [Homo sapiens] | gi\|62897113 |
| prothrombin [Homo sapiens] | gi\|339641 |
| prothrombin | |
| [Homo | |
| sapiens] | |
| prothrombin [Homo sapiens] | gi\|67624831 |
| unnamed protein product [Homo sapiens] | gi\|110623182 |

Peptide Information

| | Obsrv. | | | Start | End | |
|---|---|---|---|---|---|---|
| Calc. Mass | Mass | ±da | ±ppm | Seq. | Seq. | Sequence |
| 836.4009 | 836.415 | .0141 | 17 | 218 | 224 | GQQYQGR |
| 836.4009 | 836.415 | .0141 | 17 | 218 | 224 | GQQYQGR |
| 874.4781 | 874.4888 | .0107 | 12 | 501 | 508 | VTGWGNLK |
| 951.4894 | 951.5774 | .088 | 92 | 434 | 440 | YERNIEK |
| 971.425 | 971.4321 | .0071 | 7 | 87 | 94 | YTACETAR |
| 1148.5518 | 1148.5477 | −.0041 | −4 | 125 | 133 | SGIECQLWR |
| 1148.5518 | 1148.5477 | −.0041 | −4 | 125 | 133 | SGIECQLWR |
| 1189.579 | 1189.5732 | −.0058 | −5 | 600 | 608 | YGFYTHVFR |
| 1194.6001 | 1194.5807 | −.0194 | −16 | 354 | 363 | ELLESYIDGR |
| 1194.6001 | 1194.5807 | −.0194 | −16 | 354 | 363 | ELLESYIDGR |
| 1265.6022 | 1265.592 | −.0102 | −8 | 453 | 461 | YNWRENLDR |
| 1489.7223 | 1489.7014 | −.0209 | −14 | 597 | 608 | DGKYGFYTHVFR |
| 1542.62 | 1542.6259 | .0059 | 4 | 561 | 575 | GDACEGDSGGPF |
| | | | | | | VMK |
| 1561.7281 | 1561.6975 | −.0306 | −20 | 315 | 327 | TATSEYQTFFNPR |
| 1561.7281 | 1561.6975 | −.0306 | −20 | 315 | 327 | TATSEYQTFFNPR |
| 1645.8214 | 1645.7874 | −.034 | −21 | 385 | 399 | SPQELLCGASLISDR |
| 1698.7211 | 1698.6948 | −.0263 | −15 | 560 | 575 | RGDACEGDSGGP |
| | | | | | | FVMK |
| 1773.9164 | 1773.8727 | −.0437 | −25 | 384 | 399 | KSPQELLCGA |
| | | | | | | SLISDR |
| 1883.8956 | 1883.8428 | −.0528 | −28 | 328 | 344 | TFGSGEADCG |
| | | | | | | LRPLFEK |
| 1883.8956 | 1883.8428 | −.0528 | −28 | 328 | 344 | TFGSGEADCG |
| | | | | | | LRPLFEK |
| 1963.908 | 1963.848 | −.06 | −31 | 248 | 263 | HQDFNSAVQL |
| | | | | | | VENFCR |
| 1968.8903 | 1968.8427 | −.0476 | −24 | 100 | 117 | LAACLEGNCA |
| | | | | | | EGLGTNYR |
| 2001.9158 | 2001.8715 | −.0443 | −22 | 544 | 560 | ITDNMFCAGYK |
| | | | | | | PDEGKR |
| 2011.9906 | 2011.9392 | −.0514 | −26 | 328 | 345 | TFGSGEADCG |
| | | | | | | LRPLFEKK |
| 2017.9106 | 2017.8978 | −.0128 | −6 | 544 | 560 | ITDNMFCAGYK |
| | | | | | | PDEGKR |
| 2070.9761 | 2070.9165 | −.0596 | −29 | 199 | 217 | SEGSSVNLSPP |
| | | | | | | LEQCVP |
| | | | | | | DR |
| 2212.0122 | 2212.0205 | .0083 | 4 | 98 | 117 | DKLAACLEGNC |
| | | | | | | AEGLGT |
| | | | | | | NYR |
| 2212.0122 | 2212.0205 | .0083 | 4 | 98 | 117 | DKLAACLEGNC |
| | | | | | | AEGLGT |
| | | | | | | NYR |
| 2232.2534 | 2232.1794 | −.074 | −33 | 518 | 537 | GQPSVLQVVNL |
| | | | | | | PIVERPV |
| | | | | | | CK |
| 2232.2534 | 2232.1794 | −.074 | −33 | 518 | 537 | GQPSVLQVVNL |
| | | | | | | PIVERPV |
| | | | | | | CK |
| 2281.0991 | 2281.072 | −.0271 | −12 | 364 | 383 | IVEGSDAEIGM |
| | | | | | | SPWQVM |
| | | | | | | LFR |
| 2292.0417 | 2292.0005 | −.0412 | −18 | 179 | 198 | QECSIPVCGQD |
| | | | | | | QVTVAM |
| | | | | | | TPR |
| 2448.1428 | 2448.1128 | −.03 | −12 | 178 | 198 | RQECSIPVCGQ |
| | | | | | | DQVTVA |
| | | | | | | MTPR |
| 2888.3591 | 2888.29 | −.0691 | −24 | 199 | 224 | SEGSSVNLSPP |
| | | | | | | LEQCVP |
| | | | | | | DRGQQYQGR |
| 2888.3591 | 2888.29 | −.0691 | −24 | 199 | 224 | SEGSSVNLSPP |
| | | | | | | LEQCVP |
| | | | | | | DRGQQYQGR |

| | |
|---|---|
| coagulation factor II precursor variant | gi\|62897109 |
| [Homo sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 836.4009 | 836.415 | .0141 | 17 | 218 | 224 | GQQYQGR |
| 836.4009 | 836.415 | .0141 | 17 | 218 | 224 | GQQYQGR |
| 874.4781 | 874.4888 | .0107 | 12 | 501 | 508 | VTGWGNLK |
| 951.4894 | 951.5774 | .088 | 92 | 434 | 440 | YERNIEK |
| 971.425 | 971.4321 | .0071 | 7 | 87 | 94 | YTACETAR |
| 1148.5518 | 1148.5477 | −.0041 | −4 | 125 | 133 | SGIECQLWR |
| 1148.5518 | 1148.5477 | −.0041 | −4 | 125 | 133 | SGIECQLWR |
| 1189.579 | 1189.5732 | −.0058 | −5 | 600 | 608 | YGFYTHVFR |
| 1194.6001 | 1194.5807 | −.0194 | −16 | 354 | 363 | ELLESYIDGR |
| 1194.6001 | 1194.5807 | −.0194 | −16 | 354 | 363 | ELLESYIDGR |
| 1265.6022 | 1265.592 | −.0102 | −8 | 453 | 461 | YNWRENLDR |
| 1489.7223 | 1489.7014 | −.0209 | −14 | 597 | 608 | DGKYGFYTH |
| | | | | | | VFR |
| 1542.62 | 1542.6259 | .0059 | 4 | 561 | 575 | GDACEGDSG |
| | | | | | | GPFVMK |
| 1561.7281 | 1561.6975 | −.0306 | −20 | 315 | 327 | TATSEYQTFF |
| | | | | | | NPR |
| 1561.7281 | 1561.6975 | −.0306 | −20 | 315 | 327 | TATSEYQTFF |
| | | | | | | NPR |
| 1645.8214 | 1645.7874 | −.034 | −21 | 385 | 399 | SPQELLCGASL |
| | | | | | | ISDR |
| 1698.7211 | 1698.6948 | −.0263 | −15 | 560 | 575 | RGDACEGDSG |
| | | | | | | GPFVMK |
| 1773.9164 | 1773.8727 | −.0437 | −25 | 384 | 399 | KSPQELLCGAS |
| | | | | | | LISDR |
| 1963.908 | 1963.848 | −.06 | −31 | 248 | 263 | HQDFNSAVQL |
| | | | | | | VENFCR |
| 1968.8903 | 1968.8427 | −.0476 | −24 | 100 | 117 | LAACLEGNCAE |
| | | | | | | GLGTNYR |
| 2001.9158 | 2001.8715 | −.0443 | −22 | 544 | 560 | ITDNMFCAGYK |
| | | | | | | PDEGKR |
| 2017.9106 | 2017.8978 | −.0128 | −6 | 544 | 560 | ITDNMFCAGYK |
| | | | | | | PDEGKR |
| 2070.9761 | 2070.9165 | −.0596 | −29 | 199 | 217 | SEGSSVNLSPP |
| | | | | | | LEQCVP |
| | | | | | | DR |
| 2212.0122 | 2212.0205 | .0083 | 4 | 98 | 117 | DKLAACLEGNC |
| | | | | | | AEGLGT |
| | | | | | | NYR |
| 2212.0122 | 2212.0205 | .0083 | 4 | 98 | 117 | DKLAACLEGNC |
| | | | | | | AEGLGT |
| | | | | | | NYR |
| 2232.2534 | 2232.1794 | −.074 | −33 | 518 | 537 | GQPSVLQVVNL |
| | | | | | | PIVERPV |
| | | | | | | CK |
| 2232.2534 | 2232.1794 | −.074 | −33 | 518 | 537 | GQPSVLQVVNL |
| | | | | | | PIVERPV |
| | | | | | | CK |
| 2281.0991 | 2281.072 | −.0271 | −12 | 364 | 383 | IVEGSDAEIGM |
| | | | | | | SPWQVM |
| | | | | | | LFR |
| 2292.0417 | 2292.0005 | −.0412 | −18 | 179 | 198 | QECSIPVCGQD |
| | | | | | | QVTVAM |
| | | | | | | TPR |
| 2448.1428 | 2448.1128 | −.03 | −12 | 178 | 198 | RQECSIPVCGQ |
| | | | | | | DQVTVA |
| | | | | | | MTPR |
| 2888.3591 | 2888.29 | −.0691 | −24 | 199 | 224 | SEGSSVNLSPP |
| | | | | | | LEQCVP |
| | | | | | | DRGQQYQGR |
| 2888.3591 | 2888.29 | −.0691 | −24 | 199 | 224 | SEGSSVNLSPP |
| | | | | | | LEQCVP |
| | | | | | | DRGQQYQGR |

| | |
|---|---|
| coagulation factor II (thrombin), isoform CRA_b | gi\|119588384 |
| [Homo sapiens] | |

Peptide Information

| | | | | Start | | |
|---|---|---|---|---|---|---|
| Calc. Mass | Obsrv. Mass | ±da | ±ppm | Seq. | End Seq. | Sequence |
| 836.4009 | 836.415 | .0141 | 17 | 218 | 224 | GQQYQGR |
| 836.4009 | 836.415 | .0141 | 17 | 218 | 224 | GQQYQGR |
| 874.4781 | 874.4888 | .0107 | 12 | 501 | 508 | VTGWGNLK |
| 951.4894 | 951.5774 | .088 | 92 | 434 | 440 | YERNIEK |
| 971.425 | 971.4321 | .0071 | 7 | 87 | 94 | YTACETAR |
| 1148.5518 | 1148.5477 | −.0041 | −4 | 125 | 133 | SGIECQLWR |
| 1148.5518 | 1148.5477 | −.0041 | −4 | 125 | 133 | SGIECQLWR |
| 1194.6001 | 1194.5807 | −.0194 | −16 | 354 | 363 | ELLESYIDGR |
| | 1194.5807 | | | | | |
| 1194.6001 | 1194.5807 | −.0194 | −16 | 354 | 363 | ELLESYIDGR |
| 1265.6022 | 1265.592 | −.0102 | −8 | 453 | 461 | YNWRENLDR |
| 1561.7281 | 1561.6975 | −.0306 | −20 | 315 | 327 | TATSEYQTFFN |
| | | | | | | PR |
| 1561.7281 | 1561.6975 | −.0306 | −20 | 315 | 327 | TATSEYQTFFN |
| | | | | | | PR |
| 1645.8214 | 1645.7874 | −.034 | −21 | 385 | 399 | SPQELLCGASL |
| | | | | | | ISDR |
| 1773.9164 | 1773.8727 | −.0437 | −25 | 384 | 399 | KSPQELLCGAS |
| | | | | | | LISDR |
| 1883.8956 | 1883.8428 | −.0528 | −28 | 328 | 344 | TFGSGEADCG |
| | | | | | | LRPLFEK |
| 1883.8956 | 1883.8428 | −.0528 | −28 | 328 | 344 | TFGSGEADCG |
| | | | | | | LRPLFEK |
| 1963.908 | 1963.848 | −.06 | −31 | 248 | 263 | HQDFNSAVQL |
| | | | | | | VENFCR |
| 1968.8903 | 1968.8427 | −.0476 | −24 | 100 | 117 | LAACLEGNCAE |
| | | | | | | GLGTNYR |
| 2011.9906 | 2011.9392 | −.0514 | −26 | 328 | 345 | TFGSGEADCG |
| | | | | | | LRPLFEKK |
| 2070.9761 | 2070.9165 | −.0596 | −29 | 199 | 217 | SEGSSVNLSPP |
| | | | | | | LEQCVP |
| | | | | | | DR |
| 2212.0122 | 2212.0205 | .0083 | 4 | 98 | 117 | DKLAACLEGNC |
| | | | | | | AEGLGT |
| | | | | | | NYR |
| 2212.0122 | 2212.0205 | .0083 | 4 | 98 | 117 | DKLAACLEGNC |
| | | | | | | AEGLGT |
| | | | | | | NYR |
| 2232.2534 | 2232.1794 | −.074 | −33 | 518 | 537 | GQPSVLQVVNL |
| | | | | | | PIVERPV |
| | | | | | | CK |
| 2232.2534 | 2232.1794 | −.074 | −33 | 518 | 537 | GQPSVLQVVNL |
| | | | | | | PIVERPV |
| | | | | | | CK |
| 2281.0991 | 2281.072 | −.0271 | −12 | 364 | 383 | IVEGSDAEIGM |
| | | | | | | SPWQVM |
| | | | | | | LFR |
| 2292.0417 | 2292.0005 | −.0412 | −18 | 179 | 198 | QECSIPVCGQD |
| | | | | | | QVTVAM |
| | | | | | | TPR |
| 2448.1428 | 2448.1128 | −.03 | −12 | 178 | 198 | RQECSIPVCGQ |
| | | | | | | DQVTVA |
| | | | | | | MTPR |
| 2888.3591 | 2888.29 | −.0691 | −24 | 199 | 224 | SEGSSVNLSPP |
| | | | | | | LEQCVP |
| | | | | | | DRGQQYQGR |
| 2888.3591 | 2888.29 | −.0691 | −24 | 199 | 224 | SEGSSVNLSPP |
| | | | | | | LEQCVP |
| | | | | | | DRGQQYQGR |

| | |
|---|---|
| unnamed protein product [Homo sapiens] | gi\|194373497 |

Peptide Information

| | Obsrv. | | | Start | | |
|---|---|---|---|---|---|---|
| Calc. Mass | Mass | ±da | ±ppm | Seq. | End Seq. | Sequence |
| 836.4009 | 836.415 | .0141 | 17 | 67 | 73 | GQQYQGR |
| 836.4009 | 836.415 | .0141 | 17 | 67 | 73 | GQQYQGR |
| 874.4781 | 874.4888 | .0107 | 12 | 350 | 357 | VTGWGNLK |
| 951.4894 | 951.5774 | .088 | 92 | 283 | 289 | YERNIEK |
| 1189.579 | 1189.5732 | −.0058 | −5 | 449 | 457 | YGFYTHVFR |
| 1194.6001 | 1194.5807 | −.0194 | −16 | 203 | 212 | ELLESYIDGR |
| 1194.6001 | 1194.5807 | −.0194 | −16 | 203 | 212 | ELLESYIDGR |
| 1265.6022 | 1265.592 | −.0102 | −8 | 302 | 310 | YNWRENLDR |
| | 1265.592 | | | | | |
| 1489.7223 | 1489.7014 | −.0209 | −14 | 446 | 457 | DGKYGFYTHVFR |
| 1542.62 | 1542.6259 | .0059 | 4 | 410 | 424 | GDACEGDSGG |
| | | | | | | PFVMK |
| 1561.7281 | 1561.6975 | −.0306 | −20 | 164 | 176 | TATSEYQTFFN |
| | | | | | | PR |
| 1561.7281 | 1561.6975 | −.0306 | −20 | 164 | 176 | TATSEYQTFFN |
| | | | | | | PR |
| 1645.8214 | 1645.7874 | −.034 | −21 | 234 | 248 | SPQELLCGASL |
| | | | | | | ISDR |
| 1698.7211 | 1698.6948 | −.0263 | −15 | 409 | 424 | RGDACEGDSG |
| | | | | | | GPFVMK |
| 1773.9164 | 1773.8727 | −.0437 | −25 | 233 | 248 | KSPQELLCGAS |
| | | | | | | LISDR |
| 1883.8956 | 1883.8428 | −.0528 | −28 | 177 | 193 | TFGSGEADCG |
| | | | | | | LRPLFEK |
| 1883.8956 | 1883.8428 | −.0528 | −28 | 177 | 193 | TFGSGEADCG |
| | | | | | | LRPLFEK |
| 1963.908 | 1963.848 | −.06 | −31 | 97 | 112 | HQDFNSAVQL |
| | | | | | | VENFCR |
| 2001.9158 | 2001.8715 | −.0443 | −22 | 393 | 409 | ITDNMFCAGYK |
| | | | | | | PDEGKR |
| 2011.9906 | 2011.9392 | −.0514 | −26 | 177 | 194 | TFGSGEADCG |
| | | | | | | LRPLFEKK |
| 2017.9106 | 2017.8978 | −.0128 | −6 | 393 | 409 | ITDNMFCAGYK |
| | | | | | | PDEGKR |
| 2070.9761 | 2070.9165 | −.0596 | −29 | 48 | 66 | SEGSSVNLSPP |
| | | | | | | LEQCVP |
| | | | | | | DR |
| 2232.2534 | 2232.1794 | −.074 | −33 | 367 | 386 | GQPSVLQVVNL |
| | | | | | | PIVERPV |
| | | | | | | CK |
| 2232.2534 | 2232.1794 | −.074 | −33 | 367 | 386 | GQPSVLQVVNL |
| | | | | | | PIVERPV |
| | | | | | | CK |
| 2281.0991 | 2281.072 | −.0271 | −12 | 213 | 232 | IVEGSDAEIGM |
| | | | | | | SPWQVM |
| | | | | | | LFR |
| 2292.0417 | 2292.0005 | −.0412 | −18 | 28 | 47 | QECSIPVCGQD |
| | | | | | | QVTVAM |
| | | | | | | TPR |
| 2448.1428 | 2448.1128 | −.03 | −12 | 27 | 47 | RQECSIPVCGQ |
| | | | | | | DQVTVA |
| | | | | | | MTPR |
| 2888.3591 | 2888.29 | −.0691 | −24 | 48 | 73 | SEGSSVNLSPP |
| | | | | | | LEQCVP |
| | | | | | | DRGQQYQGR |
| 2888.3591 | 2888.29 | −.0691 | −24 | 48 | 73 | SEGSSVNLSPP |
| | | | | | | LEQCVP |
| | | | | | | DRGQQYQGR |

| | |
|---|---|
| Chain B, Crystal Structure Of | gi\|307776409 |
| Prethombin-2FRAGMENT-2 Complex | |

Peptide Information

| | | | | Start | | |
|---|---|---|---|---|---|---|
| Calc. Mass | Obsrv. Mass | ±da | ±ppm | Seq. | End Seq. | Sequence |
| 874.4781 | 874.4888 | .0107 | 12 | 187 | 194 | VTGWGNLK |
| 951.4894 | 951.5774 | .088 | 92 | 120 | 126 | YERNIEK |
| 1189.579 | 1189.5732 | −.0058 | −5 | 286 | 294 | YGFYTHVFR |
| 1194.6001 | 1194.5807 | −.0194 | −16 | 40 | 49 | ELLESYIDGR |
| 1194.6001 | 1194.5807 | −.0194 | −16 | 40 | 49 | ELLESYIDGR |
| 1265.6022 | 1265.592 | −.0102 | −8 | 139 | 147 | YNWRENLDR |
| 1489.7223 | 1489.7014 | −.0209 | −14 | 283 | 294 | DGKYGFYTHVFR |
| 1561.7281 | 1561.6975 | −.0306 | −20 | 1 | 13 | TATSEYQTFFNPR |
| | 1561.6975 | | | | | |
| 1561.7281 | 1561.6975 | −.0306 | −20 | 1 | 13 | TATSEYQTFFN |
| | | | | | | PR |
| 1645.8214 | 1645.7874 | −.034 | −21 | 71 | 85 | SPQELLCGASL |
| | | | | | | ISDR |
| 1773.9164 | 1773.8727 | −.0437 | −25 | 70 | 85 | KSPQELLCGAS |
| | | | | | | LISDR |
| 1883.8956 | 1883.8428 | −.0528 | −28 | 14 | 30 | TFGSGEADCG |
| | | | | | | LRPLFEK |
| 1883.8956 | 1883.8428 | −.0528 | −28 | 14 | 30 | TFGSGEADCG |
| | | | | | | LRPLFEK |
| 2001.9158 | 2001.8715 | −.0443 | −22 | 230 | 246 | ITDNMFCAGYK |
| | | | | | | PDEGKR |
| 2011.9906 | 2011.9392 | −.0514 | −26 | 14 | 31 | TFGSGEADCG |
| | | | | | | LRPLFEKK |
| 2017.9106 | 2017.8978 | −.0128 | −6 | 230 | 246 | ITDNMFCAGYK |
| | | | | | | PDEGKR |
| 2225.9702 | 2225.9805 | .0103 | 5 | 247 | 267 | GDACEGDAGG |
| | | | | | | PFVMKS |
| | | | | | | PFNNR |
| 2232.2534 | 2232.1794 | −.074 | −33 | 204 | 223 | GQPSVLQVVNL |
| | | | | | | PIVERPV |
| | | | | | | CK |
| 2232.2534 | 2232.1794 | −.074 | −33 | 204 | 223 | GQPSVLQVVNL |
| | | | | | | PIVERPV |
| | | | | | | CK |
| 2281.0991 | 2281.072 | −.0271 | −12 | 50 | 69 | IVEGSDAEIGM |
| | | | | | | SPWQVM |
| | | | | | | LFR |

| | |
|---|---|
| Chain L, Crystal Structure Of Human Meizothrombin | gi\|213424252 |
| Desf1 | |

Peptide Information

| | Obsrv. | | | Start | | |
|---|---|---|---|---|---|---|
| Calc. Mass | Mass | ±da | ±ppm | Seq. | End Seq. | Sequence |
| 836.4009 | 836.415 | .0141 | 17 | 13 | 19 | GQQYQGR |
| 836.4009 | 836.415 | .0141 | 17 | 13 | 19 | GQQYQGR |
| 1194.6001 | 1194.5807 | −.0194 | −16 | 149 | 158 | ELLESYIDGR |
| 1194.6001 | 1194.5807 | −.0194 | −16 | 149 | 158 | ELLESYIDGR |
| 1561.7281 | 1561.6975 | −.0306 | −20 | 110 | 122 | TATSEYQTFFNPR |
| 1561.7281 | 1561.6975 | −.0306 | −20 | 110 | 122 | TATSEYQTFFNPR |
| 1883.8956 | 1883.8428 | −.0528 | −28 | 123 | 139 | TFGSGEADCGLRP |
| | | | | | | LFEK |
| 1883.8956 | 1883.8428 | −.0528 | −28 | 123 | 139 | TFGSGEADCGLRP |
| | | | | | | LFEK |
| 1963.908 | 1963.848 | −.06 | −31 | 43 | 58 | HQDFNSAVQLVEN |
| | | | | | | FCR |
| 2011.9906 | 2011.9392 | −.0514 | −26 | 123 | 140 | TFGSGEADCGLRP |
| | | | | | | LFEKK |

| | |
|---|---|
| Chain A, Crystal Structure Of Human | gi\|311772208 |
| Prethrombin-1 | |

Peptide Information

| | | | | Start | | |
|---|---|---|---|---|---|---|
| Calc. Mass | Obsrv. Mass | ±da | ±ppm | Seq. | End Seq. | Sequence |
| 836.4009 | 836.415 | .0141 | 17 | 20 | 26 | GQQYQGR |
| 836.4009 | 836.415 | .0141 | 17 | 20 | 26 | GQQYQGR |
| 874.4781 | 874.4888 | .0107 | 12 | 303 | 310 | VTGWGNLK |
| | 874.4888 | | | | | |
| 951.4894 | 951.5774 | .088 | 92 | 236 | 242 | YERNIEK |
| 1189.579 | 1189.5732 | −.0058 | −5 | 402 | 410 | YGFYTH |
| | | | | | | VFR |
| 1194.6001 | 1194.5807 | −.0194 | −16 | 156 | 165 | ELLESYI |
| | | | | | | DGR |
| 1194.6001 | 1194.5807 | −.0194 | −16 | 156 | 165 | ELLESYIDGR |
| 1265.6022 | 1265.592 | −.0102 | −8 | 255 | 263 | YNWRENLDR |
| 1489.7223 | 1489.7014 | −.0209 | −14 | 399 | 410 | DGKYGFYTHVFR |
| 1542.62 | 1542.6259 | .0059 | 4 | 363 | 377 | GDACEGDSGG |
| | | | | | | PFVMK |
| 1645.8214 | 1645.7874 | −.034 | −21 | 187 | 201 | SPQELLCGASL |
| | | | | | | ISDR |
| 1698.7211 | 1698.6948 | −.0263 | −15 | 362 | 377 | RGDACEGDSG |
| | | | | | | GPFVMK |
| 1773.9164 | 1773.8727 | −.0437 | −25 | 186 | 201 | KSPQELLCGAS |
| | | | | | | LISDR |
| 1963.908 | 1963.848 | −.06 | −31 | 50 | 65 | HQDFNSAVQL |
| | | | | | | VENFCR |
| 2001.9158 | 2001.8715 | −.0443 | −22 | 346 | 362 | ITDNMFCAGYK |
| | | | | | | PDEGKR |
| 2017.9106 | 2017.8978 | −.0128 | −6 | 346 | 362 | ITDNMFCAGYK |
| | | | | | | PDEGKR |
| 2070.9761 | 2070.9165 | −.0596 | −29 | 1 | 19 | SEGSSVNLSPP |
| | | | | | | LEQCVP |
| | | | | | | DR |
| 2232.2534 | 2232.1794 | −.074 | −33 | 320 | 339 | GQPSVLQVVNL |
| | | | | | | PIVERPV |
| | | | | | | CK |
| 2232.2534 | 2232.1794 | −.074 | −33 | 320 | 339 | GQPSVLQVVNL |
| | | | | | | PIVERPV |
| | | | | | | CK |
| 2281.0991 | 2281.072 | −.0271 | −12 | 166 | 185 | IVEGSDAEIGM |
| | | | | | | SPWQVM |
| | | | | | | LFR |
| 2888.3591 | 2888.29 | −.0691 | −24 | 1 | 26 | SEGSSVNLSPP |
| | | | | | | LEQCVP |
| | | | | | | DRGQQYQGR |
| 2888.3591 | 2888.29 | −.0691 | −24 | 1 | 26 | SEGSSVNLSPP |
| | | | | | | LEQCVP |
| | | | | | | DRGQQYQGR |

| | |
|---|---|
| Chain C, Crystal Structure Of Na-Free | gi\|256032634 |
| Thrombin In Complex With Thrombomodulin | |

Protein Group

| | |
|---|---|
| Chain A, 2.1 Angstrom Structure Of A Nonproductive | gi\|116666779 |
| Complex Between Antithrombin, Synthetic Heparin | |
| Mimetic Sr123781 And Two S195a Thrombin | |
| Molecules | |
| Chain A, Crystal Structure Of Na-Free Thrombin In | gi\|256032632 |
| Complex With Thrombomodulin | |
| Chain A, Crystal Structure Of Native S195a Thrombin | gi\|15825720 |
| With An Unoccupied Active Site | |
| Chain C, 2.1 Angstrom Structure Of A Nonproductive | gi\|116666781 |
| Complex Between Antithrombin, Synthetic Heparin | |
| Mimetic Sr123781 And Two S195a Thrombin | |
| Molecules | |
| Chain C, Crystal Structure Of Native S195a Thrombin | gi\|15825722 |
| With An Unoccupied Active Site | |
| Chain E, Crystal Structure Of Na-Free Thrombin In | gi\|256032636 |
| Complex With Thrombomodulin | |
| Chain E, Crystal Structure Of Native S195a Thrombin | gi\|15825724 |
| With An Unoccupied Active Site | |
| Chain L, 1.6 A Structure Of The Pci-Thrombin-Heparin | gi\|185177829 |
| Complex | |
| Chain L, 2.5a Crystal Structure Of The | gi\|52695709 |
| Antithrombin-Thrombin-Heparin Ternary Complex | |

Peptide Information

| | Obsrv. | | | Start | | |
|---|---|---|---|---|---|---|
| Calc. Mass | Mass | ±da | ±ppm | Seq. | End Seq. | Sequence |
| 1194.6001 | 1194.5807 | −.0194 | −16 | 40 | 49 | ELLESYIDGR |
| 1194.6001 | 1194.5807 | −.0194 | −16 | 40 | 49 | ELLESYIDGR |
| 1561.7281 | 1561.6975 | −.0306 | −20 | 1 | 13 | TATSEYQTFFNPR |
| 1561.7281 | 1561.6975 | −.0306 | −20 | 1 | 13 | TATSEYQTFFNPR |
| 1883.8956 | 1883.8428 | −.0528 | −28 | 14 | 30 | TFGSGEADCGLRP |
| | | | | | | LFEK |
| 1883.8956 | 1883.8428 | −.0528 | −28 | 14 | 30 | TFGSGEADCGLRP |
| | | | | | | LFEK |
| 2011.9906 | 2011.9392 | −.0514 | −26 | 14 | 31 | TFGSGEADCGLRP |
| | | | | | | LFEKK |

| | |
|---|---|
| Chain A, Nonbasic Thrombin | gi\|67464458 |
| Inhibitor Complex | |

Protein Group

| | |
|---|---|
| Chain A, Crystal Structure Of | gi\|11514051 |
| Human | |
| Alpha-ThrombinRwj-51438 | |
| Complex At 1.7 A | |
| Chain E, The Isomorphous | gi\|809450 |
| Structures Of | |
| Prethrombin2, | |
| Hirugen-And Ppack-Thrombin: | |
| Changes | |
| Accompanying | |
| Activation And Exosite Binding | |
| To | |
| Thrombin | gi\|38018090 |
| prothrombin | |
| [Homo | |
| sapiens] | |

Peptide Information

| | Obsrv. | | | Start | | |
|---|---|---|---|---|---|---|
| Calc. Mass | Mass | ±da | ±ppm | Seq. | End Seq. | Sequence |
| 874.4781 | 874.4888 | .0107 | 12 | 174 | 181 | VTGWGNLK |
| 951.4894 | 951.5774 | .088 | 92 | 107 | 113 | YERNIEK |
| 1189.579 | 1189.5732 | −.0058 | −5 | 273 | 281 | YGFYTHVFR |
| 1194.6001 | 1194.5807 | −.0194 | −16 | 27 | 36 | ELLESYIDGR |
| 1194.6001 | 1194.5807 | −.0194 | −16 | 27 | 36 | ELLESYIDGR |
| 1265.6022 | 1265.592 | −.0102 | −8 | 126 | 134 | YNWRENLDR |
| 1489.7223 | 1489.7014 | −.0209 | −14 | 270 | 281 | DGKYGFYTHVFR |
| 1542.62 | 1542.6259 | .0059 | 4 | 234 | 248 | GDACEGDSGG |
| | | | | | | PFVMK |
| 1645.8214 | 1645.7874 | −.034 | −21 | 58 | 72 | SPQELLCGASL |
| | | | | | | ISDR |
| 1698.7211 | 1698.6948 | −.0263 | −15 | 233 | 248 | RGDACEGDSG |
| | | | | | | GPFVMK |
| 1773.9164 | 1773.8727 | −.0437 | −25 | 57 | 72 | KSPQELLCGAS |
| | | | | | | LISDR |
| 1883.8956 | 1883.8428 | −.0528 | −28 | 1 | 17 | TFGSGEADCG |
| | | | | | | LRPLFEK |
| 1883.8956 | 1883.8428 | −.0528 | −28 | 1 | 17 | TFGSGEADCG |
| | | | | | | LRPLFEK |
| 2001.9158 | 2001.8715 | −.0443 | −22 | 217 | 233 | ITDNMFCAGYK |
| | | | | | | PDEGKR |
| 2011.9906 | 2011.9392 | −.0514 | −26 | 1 | 18 | TFGSGEADCG |
| | | | | | | LRPLFEKK |
| 2017.9106 | 2017.8978 | −.0128 | −6 | 217 | 233 | ITDNMFCAGYK |
| | | | | | | PDEGKR |
| 2232.2534 | 2232.1794 | −.074 | −33 | 191 | 210 | GQPSVLQVVNL |
| | | | | | | PIVERPV |
| | | | | | | CK |
| 2232.2534 | 2232.1794 | −.074 | −33 | 191 | 210 | GQPSVLQVVNL |
| | | | | | | PIVERPV |
| | | | | | | CK |
| 2281.0991 | 2281.072 | −.0271 | −12 | 37 | 56 | IVEGSDAEIGM |
| | | | | | | SPWQVM |
| | | | | | | LFR |

| | |
|---|---|
| Chain A, Crystal Structure Of Thrombin In Complex | gi\|20150575 |
| With A Novel Bicyclic Lactam Inhibitor | |

Peptide Information

| | Obsrv. | | | Start | | |
|---|---|---|---|---|---|---|
| Calc. Mass | Mass | ±da | ±ppm | Seq. | End Seq. | Sequence |
| 874.4781 | 874.4888 | .0107 | 12 | 174 | 181 | VTGWGNLK |
| 951.4894 | 951.5774 | .088 | 92 | 107 | 113 | YERNIEK |
| 1189.579 | 1189.5732 | −.0058 | −5 | 273 | 281 | YGFYTHVFR |
| 1194.6001 | 1194.5807 | −.0194 | −16 | 27 | 36 | ELLESYIDGR |
| 1194.6001 | 1194.5807 | −.0194 | −16 | 27 | 36 | ELLESYIDGR |
| 1265.6022 | 1265.592 | −.0102 | −8 | 126 | 134 | YNWRENLDR |
| 1489.7223 | 1489.7014 | −.0209 | −14 | 270 | 281 | DGKYGFYTHVFR |
| 1542.62 | 1542.6259 | .0059 | 4 | 234 | 248 | GDACEGDSGG |
| | | | | | | PFVMK |
| 1645.8214 | 1645.7874 | −.034 | −21 | 58 | 72 | SPQELLCGASL |
| | | | | | | ISDR |
| 1698.7211 | 1698.6948 | −.0263 | −15 | 233 | 248 | RGDACEGDSG |
| | | | | | | GPFVMK |
| 1773.9164 | 1773.8727 | −.0437 | −25 | 57 | 72 | KSPQELLCGAS |
| | | | | | | LISDR |
| 1883.8956 | 1883.8428 | −.0528 | −28 | 1 | 17 | TFGSGEADCG |
| | | | | | | LRPLFEK |
| 1883.8956 | 1883.8428 | −.0528 | −28 | 1 | 17 | TFGSGEADCG |
| | | | | | | LRPLFEK |
| 2001.9158 | 2001.8715 | −.0443 | −22 | 217 | 233 | ITDNMFCAGYK |
| | | | | | | PDEGKR |
| 2011.9906 | 2011.9392 | −.0514 | −26 | 1 | 18 | TFGSGEADCG |
| | | | | | | LRPLFEKK |
| 2017.9106 | 2017.8978 | −.0128 | −6 | 217 | 233 | ITDNMFCAGYK |
| | | | | | | PDEGKR |
| 2232.2534 | 2232.1794 | −.074 | −33 | 191 | 210 | GQPSVLQVVNL |
| | | | | | | PIVERPV |
| | | | | | | CK |
| 2232.2534 | 2232.1794 | −.074 | −33 | 191 | 210 | GQPSVLQVVNL |
| | | | | | | PIVERPV |
| | | | | | | CK |
| 2281.0991 | 2281.072 | −.0271 | −12 | 37 | 56 | IVEGSDAEIGM |
| | | | | | | SPWQVM LFR |

| | |
|---|---|
| Chain A, Structure Of Thrombin Complexed With | gi\|11514286 |
| Selective Non-Electrophilic Inhibitors Having | |
| Cyclohexyl Moieties At | |
| P1 | |

Protein Group

| | |
|---|---|
| Chain A, Crystal Structure Of Human Alpha-Thrombin | gi\|14278416 |
| Complexed With Bch-10556 And Exosite-Directed | |
| Peptide | |

Peptide Information

| | | | | Start | | |
|---|---|---|---|---|---|---|
| Calc. Mass | Obsrv. Mass | ±da | ±ppm | Seq. | End Seq. | Sequence |
| 874.4781 | 874.4888 | .0107 | 12 | 168 | 175 | VTGWGNLK |
| 951.4894 | 951.5774 | .088 | 92 | 101 | 107 | YERNIEK |
| 1176.6412 | 1176.5833 | −.0579 | −49 | 279 | 287 | WIQKVIDQF |
| 1189.579 | 1189.5732 | −.0058 | −5 | 267 | 275 | YGFYTHVFR |
| 1194.6001 | 1194.5807 | −.0194 | −16 | 21 | 30 | ELLESYIDGR |
| 1194.6001 | 1194.5807 | −.0194 | −16 | 21 | 30 | ELLESYIDGR |
| 1265.6022 | 1265.592 | −.0102 | −8 | 120 | 128 | YNWRENLDR |
| 1489.7223 | 1489.7014 | −.0209 | −14 | 264 | 275 | DGKYGFYTHVFR |
| 1542.62 | 1542.6259 | .0059 | 4 | 228 | 242 | GDACEGDSGG |
| | | | | | | PFVMK |
| 1645.8214 | 1645.7874 | −.034 | −21 | 52 | 66 | SPQELLCGASL |
| | | | | | | ISDR |
| 1698.7211 | 1698.6948 | −.0263 | −15 | 227 | 242 | RGDACEGDSG |
| | | | | | | GPFVMK |
| 1773.9164 | 1773.8727 | −.0437 | −25 | 51 | 66 | KSPQELLCGAS |
| | | | | | | LISDR |
| 2001.9158 | 2001.8715 | −.0443 | −22 | 211 | 227 | ITDNMFCAGYK |
| | | | | | | PDEGKR |
| 2017.9106 | 2017.8978 | −.0128 | −6 | 211 | 227 | ITDNMFCAGYK |
| | | | | | | PDEGKR |
| 2232.2534 | 2232.1794 | −.074 | −33 | 185 | 204 | GQPSVLQVVNL |
| | | | | | | PIVERPV |
| | | | | | | CK |
| 2232.2534 | 2232.1794 | −.074 | −33 | 185 | 204 | GQPSVLQVVNL |
| | | | | | | PIVERPV |
| | | | | | | CK |
| 2281.0991 | 2281.072 | −.0271 | −12 | 31 | 50 | IVEGSDAEIGM |
| | | | | | | SPWQVM |
| | | | | | | LFR |

| | |
|---|---|
| Chain A, Design Of P1′ And P3′ Residues | gi\|7766843 |
| Of Trivalent Thrombin Inhibitors And Their | |
| Crystal Structures | |

Protein Group

| | |
|---|---|
| Chain A, Design Of P1′ And P3′ Residues Of Trivalent | gi\|7766841 |
| Thrombin Inhibitors And Their Crystal Structures | |

Peptide Information

| | Obsrv. | | | Start | End | |
|---|---|---|---|---|---|---|
| Calc. Mass | Mass | ±da | ±ppm | Seq. | Seq. | Sequence |
| 874.4781 | 874.4888 | .0107 | 12 | 170 | 177 | VTGWGNLK |
| 951.4894 | 951.5774 | .088 | 92 | 103 | 109 | YERNIEK |
| 1176.6412 | 1176.5833 | −.0579 | −49 | 281 | 289 | WIQKVIDQF |
| 1189.579 | 1189.5732 | −.0058 | −5 | 269 | 277 | YGFYTHVFR |
| 1194.6001 | 1194.5807 | −.0194 | −16 | 23 | 32 | ELLESYIDGR |
| 1194.6001 | 1194.5807 | −.0194 | −16 | 23 | 32 | ELLESYIDGR |
| 1265.6022 | 1265.592 | −.0102 | −8 | 122 | 130 | YNWRENLDR |
| 1489.7223 | 1489.7014 | −.0209 | −14 | 266 | 277 | DGKYGFYTHVFR |
| 1542.62 | 1542.6259 | .0059 | 4 | 230 | 244 | GDACEGDSGGPF |
| | | | | | | VMK |
| 1645.8214 | 1645.7874 | −.034 | −21 | 54 | 68 | SPQELLCGASLISDR |
| 1698.7211 | 1698.6948 | −.0263 | −15 | 229 | 244 | RGDACEGDSGGP |
| | | | | | | FVMK |
| 1773.9164 | 1773.8727 | −.0437 | −25 | 53 | 68 | KSPQELLCGASLIS |
| | | | | | | DR |
| 2001.9158 | 2001.8715 | −.0443 | −22 | 213 | 229 | ITDNMFCAGYK |
| | | | | | | PDEGKR |
| 2017.9106 | 2017.8978 | −.0128 | −6 | 213 | 229 | ITDNMFCAGYK |
| | | | | | | PDEGKR |
| 2232.2534 | 2232.1794 | −.074 | −33 | 187 | 206 | GQPSVLQVVNL |
| | | | | | | PIVERPV |
| | | | | | | CK |
| 2232.2534 | 2232.1794 | −.074 | −33 | 187 | 206 | GQPSVLQVVNL |
| | | | | | | PIVERPV |
| | | | | | | CK |
| 2281.0991 | 2281.072 | −.0271 | −12 | 33 | 52 | IVEGSDAEIGM |
| | | | | | | SPWQVM |
| | | | | | | LFR |

| | |
|---|---|
| Chain L, Crystal Structure Of The Heparin | gi\|23200174 |
| Cofactor Ii-S195a Thrombin | |
| Complex | |

Peptide Information

| | | | | Start | | |
|---|---|---|---|---|---|---|
| Calc. Mass | Obsrv. Mass | ±da | ±ppm | Seq. | End Seq. | Sequence |
| 1561.7281 | 1561.6975 | −.0306 | −20 | 1 | 13 | TATSEYQTFFNPR |
| 1561.7281 | 1561.6975 | −.0306 | −20 | 1 | 13 | TATSEYQTFFNPR |
| 1883.8956 | 1883.8428 | −.0528 | −28 | 14 | 30 | TFGSGEADCGLRP |
| | | | | | | LFEK |
| 1883.8956 | 1883.8428 | −.0528 | −28 | 14 | 30 | TFGSGEADCGLRP |
| | | | | | | LFEK |
| 2011.9906 | 2011.9392 | −.0514 | −26 | 14 | 31 | TFGSGEADCGLRP |
| | | | | | | LFEKK |

| | |
|---|---|
| Chain A, Crystal Structure Of The | gi\|27065656 |
| Anticoagulant Slow | |
| Form Of Thrombin | |

Protein Group

| | |
|---|---|
| Chain B, Crystal Structure Of The | gi\|27065657 |
| Anticoagulant Slow | |
| Form Of Thrombin | |

Peptide Information

| | Obsrv. | | | Start | | |
|---|---|---|---|---|---|---|
| Calc. Mass | Mass | ±da | ±ppm | Seq. | End Seq. | Sequence |
| 874.4781 | 874.4888 | .0107 | 12 | 168 | 175 | VTGWGNLK |
| 1176.6412 | 1176.5833 | −.0579 | −49 | 279 | 287 | WIQKVIDQF |
| 1189.579 | 1189.5732 | −.0058 | −5 | 267 | 275 | YGFYTHVFR |
| 1194.6001 | 1194.5807 | −.0194 | −16 | 21 | 30 | ELLESYIDGR |
| 1194.6001 | 1194.5807 | −.0194 | −16 | 21 | 30 | ELLESYIDGR |
| 1265.6022 | 1265.592 | −.0102 | −8 | 120 | 128 | YNWRENLDR |
| 1489.7223 | 1489.7014 | −.0209 | −14 | 264 | 275 | DGKYGFYTHVFR |
| 1542.62 | 1542.6259 | .0059 | 4 | 228 | 242 | GDACEGDSGG |
| | | | | | | PFVMK |
| | 1542.6259 | | | | | |
| 1645.8214 | 1645.7874 | −.034 | −21 | 52 | 66 | SPQELLCGASL |
| | | | | | | ISDR |
| 1698.7211 | 1698.6948 | −.0263 | −15 | 227 | 242 | RGDACEGDSG |
| | | | | | | GPFVMK |
| 1773.9164 | 1773.8727 | −.0437 | −25 | 51 | 66 | KSPQELLCGAS |
| | | | | | | LISDR |
| 2001.9158 | 2001.8715 | −.0443 | −22 | 211 | 227 | ITDNMFCAGYK |
| | | | | | | PDEGKR |
| 2017.9106 | 2017.8978 | −.0128 | −6 | 211 | 227 | ITDNMFCAGYK |
| | | | | | | PDEGKR |
| 2232.2534 | 2232.1794 | −.074 | −33 | 185 | 204 | GQPSVLQVVNL |
| | | | | | | PIVERPV |
| | | | | | | CK |
| 2232.2534 | 2232.1794 | −.074 | −33 | 185 | 204 | GQPSVLQVVNL |
| | | | | | | PIVERPV |
| | | | | | | CK |
| 2281.0991 | 2281.072 | −.0271 | −12 | 31 | 50 | IVEGSDAEIGM |
| | | | | | | SPWQVM |
| | | | | | | LFR |

Chain A, Thrombin In Complex With
Inhibitor

Protein Group

| | |
|---|---|
| | gi\|118137345 |
| Chain A, Crystal Structue Of Thrombin In Complex | gi\|51247369 |
| With | |
| A Potent P1 Heterocycle-Aryl Based Inhibitor | |
| Chain A, Crystal Structure Of Thrombin In Complex | gi\|49259293 |
| With Compound 14b | |
| Chain A, Crystal Structure Of Thrombin In Complex | gi\|170292482 |
| With Inhibitor 15 | |
| Chain A, Structure Of Thrombin Complexed With | gi\|11514212 |
| Selective Non-Electophilic Inhibitors Having | |
| Cyclohexyl | |
| Moieties At P1 | |
| Chain A, Thrombin And P2 Pyridine N-Oxide Inhibitor | gi\|67464365 |
| Complex Structure | |
| Chain A, Thrombin In Complex With An Azafluorenyl | gi\|71042452 |
| Inhibitor | |
| 23b | |
| Chain A, Thrombin In Complex With An | gi\|78101061 |
| Oxazolopyridine | |
| Inhibitor 2 | |
| Chain A, Thrombin In Complex With An | gi\|78101058 |
| Oxazolopyridine | |
| Inhibitor 21 | |

Peptide Information

| | | | | Start | | |
|---|---|---|---|---|---|---|
| Calc. Mass | Obsrv. Mass | ±da | ±ppm | Seq. | End Seq. | Sequence |
| 874.4781 | 874.4888 | .0107 | 12 | 168 | 175 | VTGWGNLK |
| 951.4894 | 951.5774 | .088 | 92 | 101 | 107 | YERNIEK |
| 1189.579 | 1189.5732 | −.0058 | −5 | 267 | 275 | YGFYTHVFR |
| 1194.6001 | 1194.5807 | −.0194 | −16 | 21 | 30 | ELLESYIDGR |
| 1194.6001 | 1194.5807 | −.0194 | −16 | 21 | 30 | ELLESYIDGR |
| 1265.6022 | 1265.592 | −.0102 | −8 | 120 | 128 | YNWRENLDR |
| 1489.7223 | 1489.7014 | −.0209 | −14 | 264 | 275 | DGKYGFYTHVFR |
| 1542.62 | 1542.6259 | .0059 | 4 | 228 | 242 | GDACEGDSGG |
| | | | | | | PFVMK |
| 1645.8214 | 1645.7874 | −.034 | −21 | 52 | 66 | SPQELLCGASL |
| | | | | | | ISDR |
| 1698.7211 | 1698.6948 | −.0263 | −15 | 227 | 242 | RGDACEGDSG |
| | | | | | | GPFVMK |
| 1773.9164 | 1773.8727 | −.0437 | −25 | 51 | 66 | KSPQELLCGAS |
| | | | | | | LISDR |
| 2001.9158 | 2001.8715 | −.0443 | −22 | 211 | 227 | ITDNMFCAGYK |
| | | | | | | PDEGKR |
| 2017.9106 | 2017.8978 | −.0128 | −6 | 211 | 227 | ITDNMFCAGYK |
| | | | | | | PDEGKR |
| 2232.2534 | 2232.1794 | −.074 | −33 | 185 | 204 | GQPSVLQVVNL |
| | | | | | | PIVERPV |
| | | | | | | CK |
| 2232.2534 | 2232.1794 | −.074 | −33 | 185 | 204 | GQPSVLQVVNL |
| | | | | | | PIVERPV |
| | | | | | | CK |
| 2281.0991 | 2281.072 | −.0271 | −12 | 31 | 50 | IVEGSDAEIGM |
| | | | | | | SPWQVM |
| | | | | | | LFR |

| | |
|---|---|
| Chain L, Selectivity At S1, H2o Displacement, | gi\|157877724 |
| Upa, Tpa, Ser190ALA190 | |
| PROTEASE, STRUCTURE- | |
| Based | |
| Drug | |
| Design | |

Protein Group

| | |
|---|---|
| Chain L, A Novel Serine Protease Inhibition Motif | gi\|157877659 |
| Involving A Multi-Centered Short Hydrogen Bonding | |
| Network At The Active | |
| Site | |
| Chain L, A Novel Serine Protease Inhibition Motif | gi\|157877654 |
| Involving A Multi-Centered Short Hydrogen Bonding | |
| Network At The Active | |
| Site | |
| Chain L, A Novel Serine Protease Inhibition Motif | gi\|157877650 |
| Involving A Multi-Centered Short Hydrogen Bonding | |
| Network At The Active | |
| Site | |
| Chain L, A Novel Serine Protease Inhibition Motif | gi\|157877646 |
| Involving A Multi-Centered Short Hydrogen Bonding | |
| Network At The Active | |
| Site | |
| Chain L, Elaborate Manifold Of Short Hydrogen Bond | gi\|157879449 |
| Arrays Mediating Binding Of Active Site-Directed | |
| Serine | |
| Protease Inhibitors | |
| Chain L, Structural Basis For Selectivity Of A Small | gi\|157838401 |
| Molecule, S1-Binding, Sub-Micromolar Inhibitor Of | |
| Urokinase Type Plasminogen Activator | |
| Chain L, Structural Basis For Selectivity Of A Small | gi\|157838398 |
| Molecule, S1-Binding, Sub-Micromolar Inhibitor Of | |
| Urokinase Type Plasminogen Activator | |
| Chain L, Structural Basis For Selectivity Of A Small | gi\|157838393 |
| Molecule, S1-Binding, Sub-Micromolar Inhibitor Of | |
| Urokinase Type Plasminogen Activator | |

Peptide Information

| | Obsrv. | | | Start | End | |
|---|---|---|---|---|---|---|
| Calc. Mass | Mass | ±da | ±ppm | Seq. | Seq. | Sequence |
| 1194.6001 | 1194.5807 | −.0194 | −16 | 27 | 36 | ELLESYIDGX |
| 1883.8956 | 1883.8428 | −.0528 | −28 | 1 | 17 | TFGSGEADCGLRPLFEK |

| | |
|---|---|
| CASP8 associated protein 2, isoform CRA_c | gi\|119568927 |
| [Homo sapiens] | |

Protein Group

| | |
|---|---|
| CASP8 associated protein 2 [Homo sapiens] | gi\|55958516 |
| CASP8 associated protein 2, isoform CRA_a [Homo | gi\|119568925 |
| sapiens] | |
| CASP8 associated protein 2, isoform CRA_c [Homo | gi\|119568929 |
| sapiens] | |
| CASP8-associated protein 2 [Homo sapiens] | gi\|212549782 |
| CASP8-associated protein 2 [Homo sapiens] | gi\|6912288 |
| CASP8-associated protein 2 [Homo sapiens] | gi\|212549532 |
| CASP8-associated protein 2 [Homo sapiens] | |
| CASP8AP2 protein [Homo sapiens] | gi\|152012538 |
| Caspase 8 associated protein 2 [Homo sapiens] | gi\|124376904 |
| Caspase 8 associated protein 2 [Homo sapiens] | gi\|124376190 |
| FLASH [Homo sapiens] | gi\|6090856 |
| FLASH homolog RIP25 [Homo sapiens] | gi\|6760665 |
| KIAA1315 protein [Homo sapiens] | gi\|20521882 |
| RecName: Full = CASP8-associated protein 2; AltName: | gi\|74721007 |
| Full = FLICE-associated huge protein | |

Peptide Information

| | | | | Star | End | |
|---|---|---|---|---|---|---|
| Calc. | Obsr | ±da | ±ppm | Seq. | Seq. | Sequence |
| 1148.5178 | 1148.5477 | .0299 | 26 | 476 | 484 | EQESKEENR |

| | | | |
|---|---|---|---|
| | 65/C14 | | Instr./Gel |
| | | | Origin |
| | [1] Sample Project | | Instrument |
| | 20110503 | | Sample |
| | | | Name |
| Protein Name- | KH13 (SEQ ID NO. KH13) | Accession No. | |

| | |
|---|---|
| unnamed protein product [Homo sapiens] | gi\|194391084 |

Peptide Information

| | Obsrv. | | | Start | | |
|---|---|---|---|---|---|---|
| Calc. Mass | Mass | ±da | ±ppm | Seq. | End Seq. | Sequence |
| 861.407 | 861.4134 | .0064 | 7 | 251 | 257 | ICVGCPR |
| 861.407 | 861.4134 | .0064 | 7 | 251 | 257 | ICVGCPR |
| 1003.5684 | 1003.5687 | .0003 | 0 | 176 | 184 | QVVAGLNFR |
| 1003.5684 | 1003.5687 | .0003 | 0 | 176 | 184 | QVVAGLNFR |
| 1030.5356 | 1030.5386 | .003 | 3 | 305 | 312 | YFIDFVAR |
| 1030.5356 | 1030.5386 | .003 | 3 | 305 | 312 | YFIDFVAR |
| 1158.6306 | 1158.6343 | .0037 | 3 | 304 | 312 | KYFIDFVAR |
| 1251.5892 | 1251.5988 | .0096 | 8 | 53 | 63 | TVGSDTFYSFK |
| 1321.653 | 1321.6519 | −.0011 | −1 | 90 | 102 | AATGECTATVG |
| | | | | | | KR |
| 1874.8781 | 1874.8669 | −.0112 | −6 | 32 | 46 | YNSQNQSNNQ |
| | | | | | | FVLYR |
| 1874.8781 | 1874.8669 | −.0112 | −6 | 32 | 46 | YNSQNQSNNQ |
| | | | | | | FVLYR |
| 2441.1038 | 2441.092 | −.0118 | −5 | 208 | 228 | SLWNGDTGEC |
| | | | | | | TDNAYIDI |
| | | | | | | QLR |
| 2512.2178 | 2512.2173 | −.0005 | 0 | 229 | 250 | IASFSQNCDIYP |
| | | | | | | GKDFVQ |
| | | | | | | PPTK |

| | |
|---|---|
| kininogen 1, isoform CRA_a [Homo sapiens] | gi\|119598585 |
| Kininogen 1 [Homo sapiens] | gi\|37748641 |

Protein Group

| | |
|---|---|
| kininogen 1 variant [Homo sapiens] | gi\|62898910 |
| kininogen 1, isoform CRA_a [Homo sapiens] | gi\|119598587 |
| kininogen [Homo sapiens] | gi\|386853 |
| kininogen [Homo sapiens] | gi\|28932976 |
| kininogen-1 isoform 2 [Homo sapiens] | gi\|4504893 |
| prepro alpha-2-thiol proteinase inhibitor [Homo sapiens] | gi\|177890 |
| unnamed protein product [Homo sapiens] | gi\|213506121 |
| unnamed protein product [Homo sapiens] | gi\|213506103 |
| unnamed protein product [Homo sapiens] | gi\|207100681 |
| unnamed protein product [Homo sapiens] | gi\|207100663 |
| unnamed protein product [Homo sapiens] | gi\|189054825 |
| unnamed protein product [Homo sapiens] | gi\|158255114 |

Peptide Information

| | Obsrv. | | | Start | End | |
|---|---|---|---|---|---|---|
| Calc. Mass | Mass | ±da | ±ppm | Seq. | Seq. | Sequence |
| 861.407 | 861.4134 | .0064 | 7 | 263 | 269 | ICVGCPR |
| 861.407 | 861.4134 | .0064 | 7 | 263 | 269 | ICVGCPR |
| 1003.5684 | 1003.5687 | .0003 | 0 | 188 | 196 | QVVAGLNFR |
| 1003.5684 | 1003.5687 | .0003 | 0 | 188 | 196 | QVVAGLNFR |
| 1030.5356 | 1030.5386 | .003 | 3 | 317 | 324 | YFIDFVAR |
| 1030.5356 | 1030.5386 | .003 | 3 | 317 | 324 | YFIDFVAR |
| 1158.6306 | 1158.6343 | .0037 | 3 | 316 | 324 | KYFIDFVAR |
| 1251.5892 | 1251.5988 | .0096 | 8 | 65 | 75 | TVGSDTFYSFK |
| | 1251.5988 | | | | | |
| 1321.653 | 1321.6519 | −.0011 | −1 | 102 | 114 | AATGECTATVGKR |
| 1874.8781 | 1874.8669 | −.0112 | −6 | 44 | 58 | YNSQNQSNNQFVLYR |
| 1874.8781 | 1874.8669 | −.0112 | −6 | 44 | 58 | YNSQNQSNNQFVLYR |
| 2441.1038 | 2441.092 | −.0118 | −5 | 220 | 240 | SLWNGDTGE |
| | | | | | | CTDNAYIDI QLR |
| 2512.2178 | 2512.2173 | −.0005 | 0 | 241 | 262 | IASFSQNCDIYPGKDFVQ |

| | |
|---|---|
| RecName: Full = Kininogen-1; AltName: | gi\|124056474 |

| | |
|---|---|
| Full = Alpha-2-thiol proteinase inhibitor; AltName: | |
| Full = Fitzgerald factor; AltName: Full = High molecular | |
| weight kininogen; Short = HMWK; AltName: | |
| Full = Williams-Fitzgerald-Flaujeac factor; Contains: | |
| RecName: Full = Kininogen-1 heavy chain; Contains: | |
| RecName: Full = T-kinin; AltName: | |
| Full = Ile-Ser-Bradykinin; Contains: RecName: | |
| Full = Bradykinin; AltName: Full = Kallidin I; Contains: | |
| RecName: Full = Lysyl-bradykinin; AltName: | |
| Full = Kallidin | |
| II; Contains: RecName: Full = Kininogen-1 light chain; | |
| PPTK | |
| Contains: RecName: Full = Low molecular weight | |
| growth-promoting factor; Flags: Precursor | |
| kininogen 1, isoform CRA_b [Homo sapiens] | gi\|119598586 |
| kininogen [Homo sapiens] | gi\|386852 |
| kininogen-1 isoform 1 [Homo sapiens] | gi\|156231037 |
| unnamed protein product [Homo sapiens] | gi\|298886629 |
| unnamed protein product [Homo sapiens] | gi\|194383526 |

Protein Group

| Calc. | Obsrv. | | | Start | | |
|---|---|---|---|---|---|---|
| Mass | Mass | ±da | ±ppm | Seq. | End Seq. | Sequence |
| 861.407 | 861.4134 | .0064 | 7 | 263 | 269 | ICVGCPR |
| 861.407 | 861.4134 | .0064 | 7 | 263 | 269 | ICVGCPR |
| 940.4747 | 940.4385 | −.0362 | −38 | 513 | 520 | NGKHNGWK |
| 1003.5684 | 1003.5687 | .0003 | 0 | 188 | 196 | QVVAGLNFR |
| 1003.5684 | 1003.5687 | .0003 | 0 | 188 | 196 | QVVAGLNFR |
| 1030.5356 | 1030.5386 | .003 | 3 | 317 | 324 | YFIDFVAR |
| 1030.5356 | 1030.5386 | .003 | 3 | 317 | 324 | YFIDFVAR |
| 1158.6306 | 1158.6343 | .0037 | 3 | 316 | 324 | KYFIDFVAR |
| 1251.5892 | 1251.5988 | .0096 | 8 | 65 | 75 | TVGSDTFYSFK |
| 1321.653 | 1321.6519 | −.0011 | −1 | 102 | 114 | AATGECTATVGKR |
| 1874.8781 | 1874.8669 | −.0112 | −6 | 44 | 58 | YNSQNQSNNQFVL [symbol] |
| 1874.8781 | 1874.8669 | −.0112 | −6 | 44 | 58 | YNSQNQSNNQFVL [symbol] |
| 2441.1038 | 2441.092 | −.0118 | −5 | 220 | 240 | SLWNGDTGECTDN [symbol] |
| | | | | | | QLR |
| 2512.2178 | 2512.2173 | −.0005 | 0 | 241 | 262 | IASFSQNCDIYPGK [symbol] |
| | | | | | | PPTK |

| | |
|---|---|
| kininogen-1 isoform 3 | gi\|262050546 |
| [Homo sapiens] | |

[symbol]

indicates data missing or illegible when filed

Peptide Information

| Calc. | Obsrv. | | | Start | End | |
|---|---|---|---|---|---|---|
| Mass | Mass | ±da | ±ppm | Seq. | Seq. | Sequence |
| 861.407 | 861.4134 | .0064 | 7 | 227 | 233 | ICVGCPR |
| 861.407 | 861.4134 | .0064 | 7 | 227 | 233 | ICVGCPR |
| 1030.5356 | 1030.5386 | .003 | 3 | 281 | 288 | YFIDFVAR |
| | 1030.5386 | | | | | |
| 1030.5356 | 1030.5386 | .003 | 3 | 281 | 288 | YFIDFVAR |
| 1158.6306 | 1158.6343 | .0037 | 3 | 280 | 288 | KYFIDFVAR |
| 1251.5892 | 1251.5988 | .0096 | 8 | 65 | 75 | TVGSDTFYSFK |
| 1321.653 | 1321.6519 | −.0011 | −1 | 102 | 114 | AATGECTATVGKR |
| 1874.8781 | 1874.8669 | −.0112 | −6 | 44 | 58 | YNSQNQSNNQFVLYR |
| 1874.8781 | 1874.8669 | −.0112 | −6 | 44 | 58 | YNSQNQSNNQFVLYR |
| 2512.2178 | 2512.2173 | −.0005 | 0 | 205 | 226 | IASFSQNCDIYPGKDFVQ |
| | | | | | | PPTK |

| | |
|---|---|
| KNG1 protein [Homo sapiens] | gi\|116283732 |

Peptide Information

| Calc. | Obsrv. | | | Start | End | |
|---|---|---|---|---|---|---|
| Mass | Mass | ±da | ±ppm | Seq. | Seq. | Sequence |
| 861.407 | 861.4134 | .0064 | 7 | 127 | 133 | ICVGCPR |
| 861.407 | 861.4134 | .0064 | 7 | 127 | 133 | ICVGCPR |
| 1003.5684 | 1003.5687 | .0003 | 0 | 52 | 60 | QVVAGLNFR |
| 1003.5684 | 1003.5687 | .0003 | 0 | 52 | 60 | QVVAGLNFR |
| 1030.5356 | 1030.5386 | .003 | 3 | 181 | 188 | YFIDFVAR |
| 1030.5356 | 1030.5386 | .003 | 3 | 181 | 188 | YFIDFVAR |
| 1158.6306 | 1158.6343 | .0037 | 3 | 180 | 188 | KYFIDFVAR |
| 2441.1038 | 2441.092 | −.0118 | −5 | 84 | 104 | SLWNGDTGECTDNAYIDI |
| | | | | | | QLR |
| 2512.2178 | 2512.2173 | −.0005 | 0 | 105 | 126 | IASFSQNCDIYPGKDFVQ |
| | | | | | | PPTK |

| | |
|---|---|
| bromodomain and PHD finger | gi\|119584376 |
| containing, 1, isoform | |
| CRA_a [Homo | |
| sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 812.3719 | 812.3501 | −.0218 | −27 | 125 | 130 | SQFMQR |
| 814.4094 | 814.3828 | −.0266 | −33 | 257 | 262 | DTIFYR |
| 823.4056 | 823.3383 | −.0673 | −82 | 2 | 8 | SYAQAQR |
| 875.4985 | 875.4634 | −.0351 | −40 | 87 | 93 | LPEVVYR |
| 940.4669 | 940.4385 | −.0284 | −30 | 124 | 130 | KSQFMQR |
| 986.5206 | 986.5446 | .024 | 24 | 453 | 459 | TWQWLPR |
| 986.5206 | 986.5446 | .024 | 24 | 453 | 459 | TWQWLPR |
| 1158.5637 | 1158.6343 | .0706 | 61 | 48 | 58 | ENTETPAATPK |
| 1875.9131 | 1875.8643 | −.0488 | −26 | 2 | 17 | SYAQAQRMVEVDLHGR |
| 2487.1936 | 2487.0796 | −.114 | −46 | 206 | 225 | ETVPDYLDHIKKPMDFFT |
| | | | | | | MK |
| 2512.3203 | 2512.2173 | −.103 | −41 | 408 | 429 | MPREGMFHHGVPIPVPP |
| | | | | | | LEVLK |

| | |
|---|---|
| tripartite motif-containing 14, isoform | gi\|119579276 |
| CRA_c [Homo | |
| sapiens] | |

Protein Group

| | |
|---|---|
| tripartite motif-containing 14, isoform | gi\|119579280 |
| CRA_c [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 821.3974 | 821.3503 | −.0471 | −57 | 267 | 272 | QSWCLK |
| 826.3512 | 826.3759 | .0247 | 30 | 222 | 228 | DCFATGR |
| 834.3951 | 834.3577 | −.0374 | −45 | 8 | 15 | QANAESSK |
| 846.425 | 846.3918 | −.0332 | −39 | 1 | 7 | MPPRSSR |
| 865.3468 | 865.4075 | .0607 | 70 | 53 | 59 | EQADSCR |
| 1030.6005 | 1030.5386 | −.0619 | −60 | 189 | 197 | LSADRLTVR |
| 1030.6005 | 1030.5386 | −.0619 | −60 | 189 | 197 | LSADRLTVR |
| 1890.0219 | 1889.8698 | −.1521 | −80 | 22 | 37 | FTELRLLLDEEEALAK |
| 2082.0034 | 2081.917 | −.0864 | −41 | 43 | 59 | NTQLTLQVYREQADSCR |

| | |
|---|---|
| unnamed protein product | gi\|21754039 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 802.4457 | 802.3769 | −.0688 | −86 | 471 | 476 | QWLVEK |
| 804.4097 | 804.3864 | −.0233 | −29 | 584 | 590 | LSDLDNK |
| 805.3839 | 805.3542 | −.0297 | −37 | 79 | 85 | FSEASHK |
| 809.4185 | 809.3559 | −.0626 | −77 | 364 | 370 | MNKTATK |
| 820.4345 | 820.3617 | −.0728 | −89 | 72 | 78 | MKDLSAR |
| 823.4196 | 823.3383 | −.0813 | −99 | 392 | 398 | SFEAELK |
| 846.4316 | 846.3918 | −.0398 | −47 | 385 | 391 | TQVEQNK |
| 859.4883 | 859.4249 | −.0634 | −74 | 404 | 410 | VQELKDK |
| | 859.4249 | | | | | |
| 865.3971 | 865.4075 | .0104 | 12 | 187 | 193 | EMEDTIK |
| 1030.5527 | 1030.5386 | −.0141 | −14 | 616 | 623 | QEIQQEKK |
| 1030.5527 | 1030.5386 | −.0141 | −14 | 616 | 623 | QEIQQEKK |
| 1251.6368 | 1251.5988 | −.038 | −30 | 689 | 698 | DFQAWLDTKK |
| 2067.9839 | 2067.9341 | −.0498 | −24 | 347 | 363 | LQKAQEESSAMMQWLQK |

| | |
|---|---|
| KIAA1274 [Homo | gi\|57209115 |
| sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 812.4162 | 812.3501 | −.0661 | −81 | 415 | 420 | HSVWQR |
| 817.4315 | 817.3644 | −.0671 | −82 | 78 | 84 | AHYTLGR |
| 835.4308 | 835.3686 | −.0622 | −74 | 593 | 599 | EGPLTYR |
| 856.5615 | 856.4853 | −.0762 | −89 | 146 | 152 | VLQKLQK |
| 859.4567 | 859.4249 | −.0318 | −37 | 364 | 370 | MVPQGRR |
| 870.5407 | 870.5418 | .0011 | 1 | 48 | 55 | AKSIIPNK |
| 986.6146 | 986.5446 | −.07 | −71 | 541 | 548 | KVVWVSLR |
| 986.6146 | 986.5446 | −.07 | −71 | 541 | 548 | KVVWVSLR |
| 1874.9832 | 1874.8669 | −.1163 | −62 | 184 | 200 | DKQNLHENLQGLGPGVR |
| 1874.9832 | 1874.8669 | −.1163 | −62 | 184 | 200 | DKQNLHENLQGLGPGVR |
| 2067.9851 | 2067.9341 | −.051 | −25 | 600 | 615 | FQTCLTMQEVFSQHRR |
| 2082.0437 | 2081.917 | −.1267 | −61 | 125 | 144 | QVQGGLTVFGMGQPSLS |
| | | | | | | GFR |

| | |
|---|---|
| paladin [Homo | gi\|166235184 |
| sapiens] | |

Protein Group

| | |
|---|---|
| KIAA1274, isoform CRA_a | gi\|119574791 |
| [Homo sapiens] | |
| KIAA1274, isoform CRA_a | gi\|119574789 |
| [Homo sapiens] | |
| RecName: | gi\|146325028 |
| Full = Paladin | |

Peptide Information

| Calc. | Obsrv. | | | Start | End | |
|---|---|---|---|---|---|---|
| Mass | Mass | ±da | ±ppm | Seq. | Seq. | Sequence |
| 812.4162 | 812.3501 | −.0661 | −81 | 415 | 420 | HSVWQR |
| 817.4315 | 817.3644 | −.0671 | −82 | 78 | 84 | AHYTLGR |
| 835.4308 | 835.3686 | −.0622 | −74 | 593 | 599 | EGPLTYR |
| 856.5615 | 856.4853 | −.0762 | −89 | 146 | 152 | VLQKLQK |
| 859.4567 | 859.4249 | −.0318 | −37 | 364 | 370 | MVPQGRR |
| 870.5407 | 870.5418 | .0011 | 1 | 48 | 55 | AKSIIPNK |
| 986.6146 | 986.5446 | −.07 | −71 | 541 | 548 | KVVWVSLR |
| 986.6146 | 986.5446 | −.07 | −71 | 541 | 548 | KVVWVSLR |
| 1874.9832 | 1874.8669 | −.1163 | −62 | 184 | 200 | DKQNLHENLQGLGPGVR |
| 1874.9832 | 1874.8669 | −.1163 | −62 | 184 | 200 | DKQNLHENLQGLGPGVR |
| 2067.9851 | 2067.9341 | −.051 | −25 | 600 | 615 | FQTCLTMQEVFSQHRR |
| 2082.0437 | 2081.917 | −.1267 | −61 | 125 | 144 | QVQGGLTVFGMGQPSLS |
| | | | | | | GFR |

| | |
|---|---|
| coiled-coil domain-containing protein | gi\|3834829284 |
| [Homo sapiens] | |

Protein Group

| | |
|---|---|
| Coiled-coil domain containing 84 | gi\|116497243 |
| [Homo sapiens] | |
| Coiled-coil domain containing 84 | gi\|116496939 |
| [Homo sapiens] | |
| Coiled-coil domain containing 84 | |
| [Homo sapiens] | |
| DLNB14 [Homo sapiens] | gi\|30314481 |
| RecName: Full = Coiled-coil domain- | gi\|74714025 |
| containing protein 84 | |
| coiled-coil domain containing 84, | gi\|119587827 |
| isoform CRA_b [Homo sapiens] | |

Peptide Information

| Calc. | Obsrv. | | | | | |
|---|---|---|---|---|---|---|
| Mass | Mass | ±da | ±ppm | Start Seq. | End Seq. | Sequence |
| 818.4189 | 818.3662 | −.0527 | −64 | 150 | 156 | EMAAQIR |
| 834.4138 | 834.3577 | −.0561 | −67 | 150 | 156 | EMAAQIR |
| 846.458 | 846.3918 | −.0662 | −78 | 19 | 25 | GHVYSRK |
| 865.4447 | 865.4075 | −.0372 | −43 | 318 | 325 | TEAAAMKK |
| 940.4258 | 940.4385 | .0127 | 14 | 136 | 143 | GLDSYEEK |
| 986.5629 | 986.5446 | −.0183 | −19 | 29 | 36 | QLKEALER |
| 986.5629 | 986.5446 | −.0183 | −19 | 29 | 36 | QLKEALER |
| 1158.6664 | 1158.6343 | −.0321 | −28 | 147 | 156 | VIKEMAAQIR |
| 1546.7643 | 1546.6572 | −.1071 | −69 | 150 | 162 | EMAAQIREVEQSR |

| | |
|---|---|
| KIAA1274, isoform CRA_b | gi\|119574790 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 812.4162 | 812.3501 | −.0661 | −81 | 415 | 420 | HSVWQR |
| 817.4315 | 817.3644 | −.0671 | −82 | 78 | 84 | AHYTLGR |
| 835.4308 | 835.3686 | −.0622 | −74 | 644 | 650 | EGPLTYR |
| 856.5615 | 856.4853 | −.0762 | −89 | 146 | 152 | VLQKLQK |
| 859.4567 | 859.4249 | −.0318 | −37 | 364 | 370 | MVPQGRR |
| 870.5407 | 870.5418 | .0011 | 1 | 48 | 55 | AKSIIPNK |
| 986.6146 | 986.5446 | −.07 | −71 | 592 | 599 | KVVWVSLR |
| 986.6146 | 986.5446 | −.07 | −71 | 592 | 599 | KVVWVSLR |
| 1874.9832 | 1874.8669 | −.1163 | −62 | 184 | 200 | DKQNLHENLQGLGPGVR |
| 1874.9832 | 1874.8669 | −.1163 | −62 | 184 | 200 | DKQNLHENLQGLGPGVR |
| 2067.9851 | 2067.9341 | −.051 | −25 | 651 | 666 | FQTCLTMQEVFSQHRR |
| 2082.0437 | 2081.917 | −.1297 | −61 | 125 | 144 | QVQGGLTVFGMGQPSLS |
| | | | | | | GFR |

| | |
|---|---|
| 5-azacytidine induced 1, isoform | gi\|119610037 |
| CRA_a [Homo | |
| sapiens] | |

Peptide Information

| | Obsrv. | | | Start | | |
|---|---|---|---|---|---|---|
| Calc. Mass | Mass | ±da | ±ppm | Seq. | End Seq. | Sequence |
| 817.3951 | 817.3644 | −.0307 | −38 | 15 | 21 | QAWEAGR |
| 823.358 | 823.3383 | −.0197 | −24 | 150 | 155 | QEFEDR |
| 864.4495 | 864.4102 | −.0393 | −45 | 1 | 7 | MELNTLK |
| 890.4578 | 890.4135 | −.0443 | −50 | 196 | 203 | EEAVSSLR |
| 1030.5316 | 1030.5386 | .007 | 7 | 25 | 32 | EEAWLLNR |
| 1030.5316 | 1030.5386 | .007 | 7 | 25 | 32 | EEAWLLNR |
| 1158.6266 | 1158.6343 | .0077 | 7 | 24 | 32 | KEEAWLLNR |
| 1251.7056 | 1251.5988 | −.1068 | −85 | 45 | 54 | DKEIELVIHR |
| 1889.9022 | 1889.8698 | −.0324 | −17 | 99 | 115 | CSELKGQLGEAEG |
| | | | | | | ENLR |

| | |
|---|---|
| RBM25 protein [Homo sapiens] | gi\|83405045 |

Peptide Information

| | Obsrv. | | | Start | | |
|---|---|---|---|---|---|---|
| Calc. Mass | Mass | ±da | ±ppm | Seq. | End Seq. | Sequence |
| 802.4529 | 802.3769 | −.076 | −95 | 118 | 125 | RVQGASGK |
| 803.4158 | 803.3585 | −.0573 | −71 | 284 | 289 | FRDTHK |
| 805.4236 | 805.3542 | −.0694 | −86 | 98 | 104 | ASDMLIR |
| 807.4359 | 807.3569 | −.079 | −98 | 241 | 246 | KEDIFR |
| | 807.3569 | | | | | |
| 821.4185 | 821.3503 | −.0682 | −83 | 98 | 104 | ASDMLIR |
| 835.4421 | 835.3686 | −.0735 | −88 | 242 | 247 | EDIFRR |
| 865.489 | 865.4075 | −.0815 | −94 | 70 | 77 | DHPGLKAK |
| 870.5407 | 870.5418 | .0011 | 1 | 209 | 216 | GAIEVLIR |
| 1889.9215 | 1889.8698 | −.0517 | −27 | 119 | 135 | VQGASGKLQAFG |
| | | | | | | FCEYK |

| | |
|---|---|
| RNA binding motif protein 25, isoform CRA_d | gi\|119601495 |
| [Homo sapiens] | |

Peptide Information

| | Obsrv. | | | | | |
|---|---|---|---|---|---|---|
| Calc. Mass | Mass | ±da | ±ppm | Start Seq. | End Seq. | Sequence |
| 802.4529 | 802.3769 | −.076 | −95 | 118 | 125 | RVQGASGK |
| 803.4158 | 803.3585 | −.0573 | −71 | 284 | 289 | FRDTHK |
| 805.4236 | 805.3542 | −.0694 | −86 | 98 | 104 | ASDMLIR |
| 807.4359 | 807.3569 | −.079 | −98 | 241 | 246 | KEDIFR |
| 821.4185 | 821.3503 | −.0682 | −83 | 98 | 104 | ASDMLIR |
| 835.4421 | 835.3686 | −.0735 | −88 | 242 | 247 | EDIFRR |
| 865.489 | 865.4075 | −.0815 | −94 | 70 | 77 | DHPGLKAK |
| 870.5407 | 870.5418 | .0011 | 1 | 209 | 216 | GAIEVLIR |
| 1889.9215 | 1889.8698 | −.0517 | −27 | 119 | 135 | VQGASGKLQA |
| | | | | | | FGFCEYK |

| | |
|---|---|
| unnamed protein product [Homo sapiens] | gi\|40982502 |

Peptide Information

| Calc. Mass | Obsrv. Mass | ±da | ±ppm | Start | End | Sequence |
|---|---|---|---|---|---|---|
| 804.3304 | 804.3864 | .056 | 70 | 93 | 98 | TMHEDR |
| 806.3751 | 806.3377 | −.0374 | −46 | 82 | 88 | DSSREGR |
| 892.463 | 892.4081 | −.0549 | −62 | 1 | 7 | MCVLELK |
| 1003.4857 | 1003.5687 | .083 | 83 | 86 | 92 | EGRYHWR |
| 1003.4857 | 1003.5687 | .083 | 83 | 86 | 92 | EGRYHWR |
| 2473.2771 | 2473.0869 | −.1902 | −77 | 8 | 28 | DARLLSPCSFPLF |
| | | | | | | NLMFF |
| | | | | | | GIK |

| | |
|---|---|
| tartrate-resistant acid phosphatase peak 1 | gi\|262924 |
| isoform 16 kda subunit, TRAcP peak 1 | |
| {N-terminal} [human, | |
| spleen, Peptide Partial, 21 aa] | |

Peptide Information

| Calc. | Obsrv. | | | Start | End | |
|---|---|---|---|---|---|---|
| Mass | Mass | ±da | ±ppm | Seq. | Seq. | Sequence |
| 808.4087 | 808.3834 | −.0253 | −31 | 15 | 21 | EDYVLVA |
| 875.4985 | 875.4634 | −.0351 | −40 | 1 | 7 | TQLSWLK |
| 1003.5934 | 1003.5687 | −.0247 | −25 | 1 | 8 | TQLSWLKK |
| 1003.5934 | 1003.5687 | −.0247 | −25 | 1 | 8 | TQLSWLKK |

| | |
|---|---|
| PRO2472 [Homo | gi\|7959907 |
| sapiens] | |

| Calc. Mass | Obsrv. Mass | ±da | ±ppm | Start | End | Sequence |
|---|---|---|---|---|---|---|
| 804.4509 | 804.3864 | −.0645 | −80 | 72 | 77 | RILSCR |
| 846.468 | 846.3918 | −.0762 | −90 | 90 | 97 | LTVPSSSR |
| 986.5319 | 986.5446 | .0127 | 13 | 83 | 89 | WVRSPWR |
| 986.6145 | 986.5446 | −.0699 | −71 | 78 | 85 | LLATKWVR |
| 2068.0718 | 2067.9341 | −.1377 | −67 | 2 | 20 | QPTLSAVSSCTA |
| | | | | | | PPRPR |
| | | | | | | SR |

| | |
|---|---|
| unnamed protein product [Homo sapiens] | gi\|40976300 |

Peptide Information

| | | | | | En | |
|---|---|---|---|---|---|---|
| Calc. Mass | Obsrv. | ±da | ±ppm | Start Seq. | Seq. | Sequence |
| 805.3774 | 805.3542 | −.0232 | −29 | 49 | 54 | HSQFMR |
| 821.3723 | 821.3503 | −.022 | −27 | 49 | 54 | HSQFMR |
| 986.4829 | 986.5446 | .0617 | 63 | 62 | 69 | YDQYVVSI |
| 986.4829 | 986.5446 | .0617 | 63 | 62 | 69 | YDQYVVSI |
| 1321.6252 | 1321.6519 | .0267 | 20 | 49 | 58 | HSQFMRLCAR |
| 1874.9834 | 1874.8669 | −.1165 | −62 | 16 | 31 | VYSPTFLALR |
| | | | | | | GSCLYK |
| 1874.9834 | 1874.8669 | −.1165 | −62 | 16 | 31 | VYSPTFLALR |
| | | | | | | GSCLYK |

| | |
|---|---|
| hCG1991338 [Homo sapiens] | gi\|119610487 |

Peptide Information

| Calc. Mass | Obsrv. Mass | ±da | ±ppm | Start | End | Sequence |
|---|---|---|---|---|---|---|
| 804.4509 | 804.3864 | −.0645 | −80 | 72 | 77 | RILSCR |
| 846.468 | 846.3918 | −.0762 | −90 | 90 | 97 | LTVPSSSR |
| 986.5319 | 986.5446 | .0127 | 13 | 83 | 89 | WVRSPWR |
| 986.6145 | 986.5446 | −.0699 | −71 | 78 | 85 | LLATKWVR |
| 2068.0718 | 2067.9341 | −.1377 | −67 | 2 | 20 | QPTLSAVSSCTAP |
| | | | | | | PRPR |
| | | | | | | SR |

| | | | |
|---|---|---|---|
| | 66/C15 | | Instr./Gel |
| | | | Origin |
| | [1] Sample Project | | Instrument |
| | 20110503 | | Sample |
| | | | Name |
| Protein Name- | KH14 (SEQ ID NO. KH14 | Accession No. | |

| | |
|---|---|
| unnamed protein product [Homo sapiens] | gi\|194391084 |

Peptide Information

| Calc. Mass | Obsrv. Mass | ±da | ±ppm | Start | End | Sequence |
|---|---|---|---|---|---|---|
| 861.407 | 861.411 | .004 | 5 | 251 | 257 | ICVGCPR |
| 861.407 | 861.411 | .004 | 5 | 251 | 257 | ICVGCPR |
| 1003.5684 | 1003.5685 | .0001 | 0 | 176 | 184 | QVVAGLNFR |
| 1003.5684 | 1003.5685 | .0001 | 0 | 176 | 184 | QVVAGLNFR |
| 1030.5356 | 1030.5359 | .0003 | 0 | 305 | 312 | YFIDFVAR |
| 1030.5356 | 1030.5359 | .0003 | 0 | 305 | 312 | YFIDFVAR |
| 1158.6306 | 1158.6351 | .0045 | 4 | 304 | 312 | KYFIDFVAR |
| 1251.5892 | 1251.5962 | .007 | 6 | 53 | 63 | TVGSDTFYSFK |
| 1321.653 | 1321.6416 | −.0114 | −9 | 90 | 102 | AATGECTATVGKR |
| 1514.6581 | 1514.6576 | −.0005 | 0 | 78 | 89 | TWQDCEYKDAAK |
| 1784.8741 | 1784.8611 | −.013 | −7 | 53 | 67 | TVGSDTFYSFKYEIK |
| 1874.8781 | 1874.863 | −.0151 | −8 | 32 | 46 | YNSQNQSNNQFV |
| | | | | | | LYR |
| 1874.8781 | 1874.863 | −.0151 | −8 | 32 | 46 | YNSQNQSNNQFV |
| | | | | | | LYR |
| 2139.0815 | 2139.1006 | .0191 | 9 | 258 | 276 | DIPTNSPELEETLT |
| | | | | | | HTITK |
| 2441.1038 | 2441.0813 | −.0225 | −9 | 208 | 228 | SLWNGDTGECTD |
| | | | | | | NAYIDI |
| | | | | | | QLR |
| 2512.2178 | 2512.2595 | .0417 | 17 | 229 | 250 | IASFSQNCDIYPGK |
| | | | | | | DFVQ |
| | | | | | | PPTK |

| | |
|---|---|
| kininogen 1, isoform CRA_a [Homo sapiens] | gi\|119598585 |

Protein Group

| | |
|---|---|
| Kininogen 1 [Homo sapiens] | gi\|37748641 |
| kininogen 1 variant [Homo sapiens] | gi\|62898910 |
| kininogen 1, isoform CRA_a [Homo sapiens] | gi\|119598587 |
| kininogen [Homo sapiens] | gi\|386853 |
| kininogen [Homo sapiens] | gi\|28932976 |
| kininogen-1 isoform 2 [Homo sapiens] | gi\|4504893 |
| prepro alpha-2-thiol proteinase inhibitor [Homo sapiens] | gi\|177890 |
| unnamed protein product [Homo sapiens] | gi\|213506121 |
| unnamed protein product [Homo sapiens] | gi\|213506103 |
| unnamed protein product [Homo sapiens] | gi\|207100681 |
| unnamed protein product [Homo sapiens] | gi\|207100663 |
| unnamed protein product [Homo sapiens] | gi\|189054825 |
| unnamed protein product [Homo sapiens] | gi\|158255114 |
| unnamed protein product [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 861.407 | 861.411 | .004 | 5 | 263 | 269 | ICVGCPR |
| 861.407 | 861.411 | .004 | 5 | 263 | 269 | ICVGCPR |
| 1003.5684 | 1003.5685 | .0001 | 0 | 188 | 196 | QVVAGLNFR |
| 1003.5684 | 1003.5685 | .0001 | 0 | 188 | 196 | QVVAGLNFR |
| 1030.5356 | 1030.5359 | .0003 | 0 | 317 | 324 | YFIDFVAR |
| 1030.5356 | 1030.5359 | .0003 | 0 | 317 | 324 | YFIDFVAR |
| 1158.6306 | 1158.6351 | .0045 | 4 | 316 | 324 | KYFIDFVAR |
| 1251.5892 | 1251.5962 | .007 | 6 | 65 | 75 | TVGSDTFYSFK |
| 1321.653 | 1321.6416 | −.0114 | −9 | 102 | 114 | AATGECTATVGKR |
| 1514.6581 | 1514.6576 | −.0005 | 0 | 90 | 101 | TWQDCEYKDAAK |
| 1784.8741 | 1784.8611 | −.013 | −7 | 65 | 79 | TVGSDTFYSFKYEIK |
| 1874.8781 | 1874.863 | −.0151 | −8 | 44 | 58 | YNSQNQSNNQFV |
| | | | | | | LYR |
| 1874.8781 | 1874.863 | −.0151 | −8 | 44 | 58 | YNSQNQSNNQFV |
| | | | | | | LYR |
| 2139.0815 | 2139.1006 | .0191 | 9 | 270 | 288 | DIPTNSPELEETLT |
| | | | | | | HTITK |
| 2441.1038 | 2441.0813 | −.0225 | −9 | 220 | 240 | SLWNGDTGECTD |
| | | | | | | NAYIDI |
| | | | | | | QLR |
| 2512.2178 | 2512.2595 | .0417 | 17 | 241 | 262 | IASFSQNCDIYPGK |
| | | | | | | DFVQ |
| | | | | | | PPTK |

Protein Group

| | |
|---|---|
| RecName: Full = Kininogen-1; AltName: | gi\|124056474 |
| Full = Alpha-2-thiol proteinase | |
| inhibitor; AltName: Full = Fitzgerald | |
| factor; AltName: Full = High | |
| molecular weight kininogen; | |
| Short = HMWK; | |
| AltName: | |
| Full = Williams-Fitzgerald-Flaujeac | |
| factor; Contains: RecName: | |
| Full = Kininogen-1 heavy chain; | |
| Contains: RecName: Full = T-kinin; | |
| AltName: | |
| Full = Ile-Ser- | |
| Bradykinin; Contains: | |
| RecName: | |
| Full = Bradykinin; AltName: Full = Kallidin I; Contains: | |
| RecName: Full = Lysyl- | |
| bradykinin; AltName: | |
| Full = Kallidin | |
| II; Contains: RecName: Full = Kininogen-1 light chain; | |
| Contains: RecName: Full = Low | |
| molecular weight growth- | |
| promoting factor; Flags: | |
| Precursor | |
| kininogen 1, isoform CRA_b [Homo sapiens] | gi\|119598586 |
| kininogen 1, isoform | |
| CRA_b [Homo sapiens] | |
| kininogen [Homo sapiens] | gi\|386852 |
| kininogen-1 isoform 1 [Homo sapiens] | gi\|156231037 |
| unnamed protein product [Homo sapiens] | gi\|298886629 |
| unnamed protein product [Homo sapiens] | gi\|194383526 |

Peptide Information

| | Obsrv. | | | | End | |
|---|---|---|---|---|---|---|
| Calc. Mass | Mass | ±da | ±ppm | Start Seq. | Seq. | Sequence |
| 861.407 | 861.411 | .004 | 5 | 263 | 269 | ICVGCPR |
| 861.407 | 861.411 | .004 | 5 | 263 | 269 | ICVGCPR |
| 1003.5684 | 1003.5685 | .0001 | 0 | 188 | 196 | QVVAGLNFR |
| 1003.5684 | 1003.5685 | .0001 | 0 | 188 | 196 | QVVAGLNFR |
| 1030.5356 | 1030.5359 | .0003 | 0 | 317 | 324 | YFIDFVAR |
| 1030.5356 | 1030.5359 | .0003 | 0 | 317 | 324 | YFIDFVAR |
| 1158.6306 | 1158.6351 | .0045 | 4 | 316 | 324 | KYFIDFVAR |
| 1251.5892 | 1251.5962 | .007 | 6 | 65 | 75 | TVGSDTFYSFK |
| 1321.653 | 1321.6416 | −.0114 | −9 | 102 | 114 | AATGECTATVG |
| | | | | | | KR |
| 1514.6581 | 1514.6576 | −.0005 | 0 | 90 | 101 | TWQDCEYKDA |
| | | | | | | AK |
| 1784.8741 | 1784.8611 | −.013 | −7 | 65 | 79 | TVGSDTFYSFK |
| | | | | | | YEIK |
| 1874.8781 | 1874.863 | −.0151 | −8 | 44 | 58 | YNSQNQSNNQ |
| | | | | | | FVLYR |
| 1874.8781 | 1874.863 | −.0151 | −8 | 44 | 58 | YNSQNQSNNQ |
| | | | | | | FVLYR |
| 2139.0815 | 2139.1006 | .0191 | 9 | 270 | 288 | DIPTNSPELEET |
| | | | | | | LTHTITK |
| 2441.1038 | 2441.0813 | −.0225 | −9 | 220 | 240 | SLWNGDTGEC |
| | | | | | | TDNAYIDI |
| | | | | | | QLR |
| 2512.2178 | 2512.2595 | .0417 | 17 | 241 | 262 | IASFSQNCDIYP |
| | | | | | | GKDFVQ |
| | | | | | | PPTK |

| | |
|---|---|
| kininogen-1 isoform 3 [Homo sapiens] | gi\|262050546 |

Peptide Information

| | | | | Start | | |
|---|---|---|---|---|---|---|
| Calc. Mass | Obsrv. Mass | ±da | ±ppm | Seq. | End Seq. | Sequence |
| 861.407 | 861.411 | .004 | 5 | 227 | 233 | ICVGCPR |
| 861.407 | 861.411 | .004 | 5 | 227 | 233 | ICVGCPR |
| 1030.5356 | 1030.5359 | .0003 | 0 | 281 | 288 | YFIDFVAR |
| 1030.5356 | 1030.5359 | .0003 | 0 | 281 | 288 | YFIDFVAR |
| 1158.6306 | 1158.6351 | .0045 | 4 | 280 | 288 | KYFIDFVAR |
| 1251.5892 | 1251.5962 | .007 | 6 | 65 | 75 | TVGSDTFYSFK |
| 1321.653 | 1321.6416 | −.0114 | −9 | 102 | 114 | AATGECTATV |
| | | | | | | GKR |
| 1514.6581 | 1514.6576 | −.0005 | 0 | 90 | 101 | TWQDCEYKDA |
| | | | | | | AK |
| 1784.8741 | 1784.8611 | −.013 | −7 | 65 | 79 | TVGSDTFYSFK |
| | | | | | | YEIK |
| 1874.8781 | 1874.863 | −.0151 | −8 | 44 | 58 | YNSQNQSNNQ |
| | | | | | | FVLYR |
| 1874.8781 | 1874.863 | −.0151 | −8 | 44 | 58 | YNSQNQSNNQ |
| | | | | | | FVLYR |
| 2139.0815 | 2139.1006 | .0191 | 9 | 234 | 252 | DIPTNSPELEE |
| | | | | | | TLTHTITK |
| 2512.2178 | 2512.2595 | .0417 | 17 | 205 | 226 | IASFSQNCDIY |
| | | | | | | PGKDFVQ |
| | | | | | | PPTK |

| | |
|---|---|
| KNG1 protein [Homo sapiens] | gi\|116283732 |

Peptide Information

| | Obsrv. | | | Start | | |
|---|---|---|---|---|---|---|
| Calc. Mass | Mass | ±da | ±ppm | Seq. | End Seq. | Sequence |
| 861.407 | 861.411 | .004 | 5 | 127 | 133 | ICVGCPR |
| 861.407 | 861.411 | .004 | 5 | 127 | 133 | ICVGCPR |
| 1003.5684 | 1003.5685 | .0001 | 0 | 52 | 60 | QVVAGLNFR |
| 1003.5684 | 1003.5685 | .0001 | 0 | 52 | 60 | QVVAGLNFR |
| 1030.5356 | 1030.5359 | .0003 | 0 | 181 | 188 | YFIDFVAR |
| 1030.5356 | 1030.5359 | .0003 | 0 | 181 | 188 | YFIDFVAR |
| 1158.6306 | 1158.6351 | .0045 | 4 | 180 | 188 | KYFIDFVAR |
| 2139.0815 | 2139.1006 | .0191 | 9 | 134 | 152 | DIPTNSPELEETLT |
| | | | | | | HTITK |
| 2441.1038 | 2441.0813 | −.0225 | −9 | 84 | 104 | SLWNGDTGECTD |
| | | | | | | NAYIDI |
| | | | | | | QLR |
| 2512.2178 | 2512.2595 | .0417 | 17 | 105 | 126 | IASFSQNCDIYPGK |
| | | | | | | DFVQ |
| | | | | | | PPTK |

| | |
|---|---|
| unnamed protein product [Homo sapiens] | gi\|11191031 |

Peptide Information

| | Obsrv. | | | Start | | |
|---|---|---|---|---|---|---|
| Calc. Mass | Mass | ±da | ±ppm | Seq. | End Seq. | Sequence |
| 813.4213 | 813.3471 | −.0742 | −91 | 58 | 65 | DGQLAGPR |
| 857.4515 | 857.424 | −.0275 | −32 | 112 | 117 | YYVKER |
| 865.3785 | 865.4213 | .0428 | 49 | 232 | 239 | TESALTED |
| 870.5083 | 870.5385 | .0302 | 35 | 109 | 115 | LGKYYVK |
| 986.491 | 986.5446 | .0536 | 54 | 1 | 8 | MPLGHIMR |
| 986.491 | 986.5446 | .0536 | 54 | 1 | 8 | MPLGHIMR |
| 2095.9429 | 2095.9539 | .011 | 5 | 206 | 222 | YAMTVWYFDAEE |
| | | | | | | RAEAK |
| 2473.2551 | 2473.0913 | −.1638 | −66 | 173 | 192 | SFIADVEPIFDRLL |
| | | | | | | FFWSDR |
| 2473.2551 | 2473.0913 | −.1638 | −66 | 173 | 192 | SFIADVEPIFDRLL |
| | | | | | | FFWSDR |

| | |
|---|---|
| egl nine homolog 3 (C. elegans) | gi\|119586333 |
| [Homo sapiens] | |

Protein Group

| | |
|---|---|
| EGLN3 protein [Homo sapiens] | gi\|130267189 |
| EGLN3 protein [Homo sapiens] | gi\|14547150 |
| EGLN3 protein [Homo sapiens] | |
| Egl nine homolog 3 (C. elegans) [Homo sapiens] | gi\|127798639 |
| Egl nine homolog 3 (C. elegans) [Homo sapiens] | gi\|127796436 |
| Egl nine homolog 3 (C. elegans) [Homo sapiens] | gi\|77748083 |
| RecName: Full = Egl nine homolog 3; AltName: | gi\|32129515 |
| Full = HPH-1; AltName: Full = Hypoxia- | |
| inducible factor prolyl hydroxylase 3; | |
| Short = HIF-PH3; Short = HIF- | |
| prolyl | |
| hydroxylase 3; Short = HPH-3; AltName: Full = Prolyl | |
| hydroxylase domain-containing | |
| protein 3; Short = PHD3 | |
| egl nine homolog 3 [Homo sapiens] | gi\|11545787 |
| unnamed protein product [Homo | gi\|10437756 |
| sapiens] | |
| unnamed protein product [Homo sapiens] | gi\|40216521 |
| unnamed protein product [Homo sapiens] | gi\|25897867 |
| unnamed protein product [Homo sapiens] | gi\|193784739 |

Peptide Information

| | | | | Start | End | |
|---|---|---|---|---|---|---|
| Calc. Mass | Obsrv. Mass | ±da | ±ppm | Seq. | Seq. | Sequence |
| 813.4213 | 813.3471 | −.0742 | −91 | 58 | 65 | DGQLAGPR |
| 857.4515 | 857.424 | −.0275 | −32 | 112 | 117 | YYVKER |
| 865.3785 | 865.4213 | .0428 | 49 | 232 | 239 | TESALTED |
| 870.5083 | 870.5385 | .0302 | 35 | 109 | 115 | LGKYYVK |
| 986.491 | 986.5446 | .0536 | 54 | 1 | 8 | MPLGHIMR |
| 986.491 | 986.5446 | .0536 | 54 | 1 | 8 | MPLGHIMR |
| 2095.9429 | 2095.9539 | .011 | 5 | 206 | 222 | YAMTVWYFDAEE |
| | | | | | | RAEAK |
| 2473.2551 | 2473.0913 | −.1638 | −66 | 173 | 192 | SFIADVEPIFDRLL |
| | | | | | | FFWSDR |
| 2473.2551 | 2473.0913 | −.1638 | −66 | 173 | 192 | SFIADVEPIFDRLL |
| | | | | | | FFWSDR |
| | 2473.0913 | | | | | |

| | |
|---|---|
| unnamed protein product [Homo sapiens] | gi\|40976466 |

Peptide Information

| | | | | Start | | |
|---|---|---|---|---|---|---|
| Calc. Mass | Obsrv. Mass | ±da | ±ppm | Seq. | End Seq. | Sequence |
| 818.4553 | 818.3741 | −.0812 | −99 | 13 | 18 | LLERCK |
| 824.376 | 824.3765 | .0005 | 1 | 19 | 24 | YWPACK |
| 1784.7479 | 1784.8611 | .1132 | 63 | 25 | 39 | NGDECAYHHPISP |
| | | | | | | CK |
| 1858.0103 | 1857.8372 | −.1731 | −93 | 1 | 16 | MSELSVAQKPEKL |
| | | | | | | LER |
| 2081.9531 | 2081.9285 | −.0246 | −12 | 59 | 75 | YDAKCTKPDCPFT |
| | | | | | | HVSR |
| 2081.9531 | 2081.9285 | −.0246 | −12 | 59 | 75 | YDAKCTKPDCPFT |
| | | | | | | HVSR |

| | |
|---|---|
| unnamed protein product [Homo sapiens] | gi\|40977232 |

Peptide Information

| | | | | Start | | |
|---|---|---|---|---|---|---|
| Calc. Mass | Obsrv. Mass | ±da | ±ppm | Seq. | End Seq. | Sequence |
| 1002.5102 | 1002.5437 | .0335 | 33 | 55 | 63 | APEETIQSK |
| 1030.4888 | 1030.5359 | .0471 | 46 | 2 | 9 | VSHTFHMR |
| 1030.4888 | 1030.5359 | .0471 | 46 | 2 | 9 | VSHTFHMR |
| 1161.5293 | 1161.455 | −.0743 | −64 | 1 | 9 | MVSHTFHMR |
| 1889.8665 | 1889.8593 | −.0072 | −4 | 80 | 96 | VTSPSPQQNPSSP |
| | | | | | | SWQY |
| 1889.8665 | 1889.8593 | −.0072 | −4 | 80 | 96 | VTSPSPQQNPSSP |
| | | | | | | SWQY |

| | |
|---|---|
| mitochondrial NADP(+)-dependent malic enzyme 3 | gi\|37904424 |
| [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 861.4802 | 861.411 | −.0692 | −80 | 186 | 192 | HQRVHGK |
| 861.4802 | 861.411 | −.0692 | −80 | 186 | 192 | HQRVHGK |
| 865.4414 | 865.4213 | −.0201 | −23 | 2 | 8 | AFTLEER |
| 1012.4768 | 1012.5323 | .0555 | 55 | 1 | 8 | MAFTLEER |
| 1030.5139 | 1030.5359 | .022 | 21 | 31 | 37 | IMRYYER |
| 1030.5139 | 1030.5359 | .022 | 21 | 31 | 37 | IMRYYER |
| 1889.8474 | 1889.8593 | .0119 | 6 | 193 | 208 | AYDDLLDEFMQAVTDK |
| 1889.8474 | 1889.8593 | .0119 | 6 | 193 | 208 | AYDDLLDEFMQAVTDK |
| 2067.8535 | 2067.8975 | .044 | 21 | 234 | 249 | NKYCMFNDDIQDDFSR |
| 2096.0693 | 2095.9539 | −.1154 | −55 | 38 | 54 | QQSDLDKYIILMTLQDR |

| | |
|---|---|
| unnamed protein product [Homo sapiens] | gi\|193786868 |

Peptide Information

| | Obsrv. | | | Start | End | |
|---|---|---|---|---|---|---|
| Calc. Mass | Mass | ±da | ±ppm | Seq. | Seq. | Sequence |
| 806.4189 | 806.3558 | −.0631 | −78 | 38 | 44 | VMQIGSR |
| 815.4734 | 815.3965 | −.0769 | −94 | 195 | 201 | LVQVNSR |
| 865.4414 | 865.4213 | −.0201 | −23 | 2 | 9 | QYIGEGAK |
| 1012.4768 | 1012.5323 | .0555 | 55 | 1 | 9 | MQYIGEGAK |
| 1030.4412 | 1030.5359 | .0947 | 92 | 14 | 20 | ECQYQFR |
| 1030.4412 | 1030.5359 | .0947 | 92 | 14 | 20 | ECQYQFR |
| 1784.7278 | 1784.8611 | .1333 | 75 | 266 | 277 | CHCKFHWCCYVK |
| 2487.1497 | 2487.0925 | −.0572 | −23 | 202 | 222 | FNSPTTQDLVYIDP |
| | | | | | | SPDY |
| | | | | | | CVR |
| 2500.9658 | 2501.1904 | .2246 | 90 | 239 | 259 | TSEGMDGCELMC |
| | | | | | | CGRG |
| | | | | | | YDQFK |

| | |
|---|---|
| Br140 [Homo sapiens] | gi\|190352 |

Peptide Information

| | | | | Start | End | |
|---|---|---|---|---|---|---|
| Calc. Mass | Obsrv. Mass | ±da | ±ppm | Seq. | Seq. | Sequence |
| 815.437 | 815.3965 | −.0405 | −50 | 723 | 730 | EQGGAVVR |
| 848.3679 | 848.3872 | .0193 | 23 | 569 | 575 | NCDQVGR |
| 861.3883 | 861.411 | .0227 | 26 | 990 | 996 | NDCSLPR |
| 861.3883 | 861.411 | .0227 | 26 | 990 | 996 | NDCSLPR |
| 875.4468 | 875.4574 | .0106 | 12 | 793 | 800 | LDEVNASK |
| 986.52 | 986.5446 | .0246 | 25 | 1058 | 1066 | GVGHSMVRK |
| 986.5206 | 986.5446 | .024 | 24 | 1153 | 1159 | TWQWLPR |
| 1003.5101 | 1003.5685 | .0584 | 58 | 319 | 326 | RCLQSPSR |
| 1003.5101 | 1003.5685 | .0584 | 58 | 319 | 326 | RCLQSPSR |
| 1158.5637 | 1158.6351 | .0714 | 62 | 137 | 147 | ENTETPAATPK |
| 1211.6532 | 1211.7167 | .0635 | 52 | 711 | 720 | DTIFYRAAVR |
| 1874.8927 | 1874.863 | −.0297 | −16 | 837 | 854 | HGPSSRGSLTPHP |
| | | | | | | AACDK |
| 1874.8927 | 1874.863 | −.0297 | −16 | 837 | 854 | HGPSSRGSLTPHP |
| | | | | | | AACDK |
| 2096.1362 | 2095.9539 | −.1823 | −87 | 1111 | 1129 | EGMFHHGVPIPVP |
| | | | | | | PLEVLK |
| 2512.3203 | 2512.2595 | −.0608 | −24 | 1108 | 1129 | MPREGMFHHGVPI |
| | | | | | | PVPP |
| | | | | | | LEVLK |

| | |
|---|---|
| DYNC2H1 variant protein [Homo sapiens] | gi\|166788546 |

Peptide Information

| | | | | Start | End | |
|---|---|---|---|---|---|---|
| Calc. Mass | Obsrv. Mass | ±da | ±ppm | Seq. | Seq. | Sequence |
| 804.421 | 804.391 | −.03 | −37 | 3603 | 3609 | KADSQQK |
| 806.4076 | 806.3558 | −.0518 | −64 | 436 | 441 | ELMLER |
| 815.4522 | 815.3965 | −.0557 | −68 | 2614 | 2620 | GLIHYGR |
| 818.4254 | 818.3741 | −.0513 | −63 | 1183 | 1189 | LQSEVDK |
| 820.3584 | 820.3719 | .0135 | 16 | 123 | 128 | DQEWSR |
| 827.4443 | 827.3964 | −.0479 | −58 | 3008 | 3014 | MPPDVIR |
| 844.4774 | 844.3963 | −.0811 | −96 | 522 | 528 | DLLDQLK |
| 848.4658 | 848.3872 | −.0786 | −93 | 1994 | 2001 | AALCKTGK |
| 861.4862 | 861.411 | −.0752 | −87 | 1307 | 1313 | CLLQSLK |
| 861.4862 | 861.411 | −.0752 | −87 | 1307 | 1313 | CLLQSLK |
| 873.4689 | 873.3854 | −.0835 | −96 | 714 | 719 | QQQRWK |
| 875.4985 | 875.4574 | −.0411 | −47 | 3623 | 3630 | SWAVATLK |
| 985.5287 | 985.5172 | −.0115 | −12 | 1629 | 1635 | QLRFYMK |
| 1002.5102 | 1002.5437 | .0335 | 33 | 3422 | 3429 | LLQQEEDK |
| 1003.524 | 1003.5685 | .0445 | 44 | 2075 | 2083 | LLTMPSGER |
| 1003.524 | 1003.5685 | .0445 | 44 | 2075 | 2083 | LLTMPSGER |
| 1012.5462 | 1012.5323 | −.0139 | −14 | 2684 | 2691 | GYELKQFK |
| 1158.5824 | 1158.6351 | .0527 | 45 | 1173 | 1182 | VEEHSVMTVK |
| 1514.7383 | 1514.6576 | −.0807 | −53 | 3023 | 3035 | LMGIFDTSWVSMK |
| 1546.728 | 1546.6423 | −.0857 | −55 | 3023 | 3035 | LMGIFDTSWVSMK |
| 1546.728 | 1546.6423 | −.0857 | −55 | 3023 | 3035 | LMGIFDTSWVSMK |
| 1857.9963 | 1857.8372 | −.1591 | −86 | 1399 | 1413 | NSLLTILDQLQRC |
| | | | | | | QR |
| 1874.787 | 1874.863 | .076 | 41 | 2803 | 2817 | CQVLWMEGWSNS |
| | | | | | | SMK |
| 1874.9569 | 1874.863 | −.0939 | −50 | 859 | 874 | EWIVIGQVDMEAL |
| | | | | | | VEK |
| 2068.0974 | 2067.8975 | −.1999 | −97 | 1854 | 1870 | ELLTPQQHYDWG |
| | | | | | | LRALK |
| 2081.9858 | 2081.9285 | −.0573 | −28 | 1015 | 1030 | WDKFELMMESHQ |
| | | | | | | LMIK |
| 2081.9858 | 2081.9285 | −.0573 | −28 | 1015 | 1030 | WDKFELMMESHQ |
| | | | | | | LMIK |
| 2441.0806 | 2441.0813 | .0007 | 0 | 2929 | 2950 | QDEADAALQMITV |
| | | | | | | SMQD |
| | | | | | | ASEQK |
| 2473.197 | 2473.0913 | −.1057 | −43 | 4123 | 4142 | CPLAWQSKWEGP |
| | | | | | | EDPL |
| | | | | | | QYLR |
| 2473.2258 | 2473.0913 | −.1345 | −54 | 168 | 188 | GILTPSDEFQFWIE |
| | | | | | | QAHR |
| | | | | | | GNK |
| 2501.2671 | 2501.1904 | −.0767 | −31 | 3374 | 3396 | NPNPFIPPDAASIV |
| | | | | | | TEVNF |
| | | | | | | TTTR |
| 2512.1956 | 2512.2595 | .0639 | 25 | 1018 | 1037 | FELMMESHQLMIK |
| | | | | | | DQIEV |
| | 2512.2595 | | | | | MK |

| | |
|---|---|
| PREDICTED: putative zinc finger protein | gi\|239752112 |
| ENSP00000328166-like, partial [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 825.3923 | 825.3739 | −.0184 | −22 | 268 | 273 | LNEYMR |
| 827.324 | 827.3964 | .0724 | 88 | 311 | 316 | FEECDK |
| 904.5073 | 904.4672 | −.0401 | −44 | 31 | 37 | KKPWSMK |
| 1012.4516 | 1012.5323 | .0807 | 80 | 2 | 9 | DVMSQNYR |
| 1015.6259 | 1015.5489 | −.077 | −76 | 377 | 385 | SLGLTIQKR |
| 1175.4821 | 1175.4708 | −.0113 | −10 | 1 | 9 | MDVMSQNYR |
| 1211.6532 | 1211.7167 | .0635 | 52 | 413 | 422 | IVHTGEKHYK |
| 1514.7791 | 1514.6576 | −.1215 | −80 | 401 | 412 | AFYWSLSFTKHK |
| 2096.0078 | 2095.9539 | −.0539 | −26 | 93 | 111 | VQEGGYNGLNQC |
| | | | | | | LSITQ |
| | | | | | | SK |
| 2138.9668 | 2139.1006 | .1338 | 63 | 283 | 300 | CEECGKTFMQPS |
| | | | | | | GLTLHK |

| | |
|---|---|
| cytoplasmic dynein 2 heavy chain 1 isoform 2 | gi\|122937398 |
| [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 804.421 | 804.391 | −.03 | −37 | 3610 | 3616 | KADSQQK |
| 806.4076 | 806.3558 | −.0518 | −64 | 436 | 441 | ELMLER |
| 815.4522 | 815.3965 | −.0557 | −68 | 2614 | 2620 | GLIHYGR |
| 818.4254 | 818.3741 | −.0513 | −63 | 1183 | 1189 | LQSEVDK |
| 820.3584 | 820.3719 | .0135 | 16 | 123 | 128 | DQEWSR |
| 827.4443 | 827.3964 | −.0479 | −58 | 3008 | 3014 | MPPDVIR |
| 844.4774 | 844.3963 | −.0811 | −96 | 522 | 528 | DLLDQLK |
| 848.4658 | 848.3872 | −.0786 | −93 | 1994 | 2001 | AALCKTGK |
| 861.4862 | 861.411 | −.0752 | −87 | 1307 | 1313 | CLLQSLK |
| 861.4862 | 861.411 | −.0752 | −87 | 1307 | 1313 | CLLQSLK |
| 873.4689 | 873.3854 | −.0835 | −96 | 714 | 719 | QQQRWK |
| 875.4985 | 875.4574 | −.0411 | −47 | 3630 | 3637 | SWAVATLK |
| 985.5287 | 985.5172 | −.0115 | −12 | 1629 | 1635 | QLRFYMK |
| 1002.5102 | 1002.5437 | .0335 | 33 | 3429 | 3436 | LLQQEEDK |
| 1003.524 | 1003.5685 | .0445 | 44 | 2075 | 2083 | LLTMPSGER |
| | 1003.5685 | | | | | |
| 1003.524 | 1003.5685 | .0445 | 44 | 2075 | 2083 | LLTMPSGER |
| 1012.5462 | 1012.5323 | −.0139 | −14 | 2684 | 2691 | GYELKQFK |
| 1158.5824 | 1158.6351 | .0527 | 45 | 1173 | 1182 | VEEHSVMTVK |
| 1514.7383 | 1514.6576 | −.0807 | −53 | 3023 | 3035 | LMGIFDTSWVSMK |
| 1546.728 | 1546.6423 | −.0857 | −55 | 3023 | 3035 | LMGIFDTSWVSMK |
| 1546.728 | 1546.6423 | −.0857 | −55 | 3023 | 3035 | LMGIFDTSWVS |
| | | | | | | MK |
| 1874.787 | 1874.863 | .076 | 41 | 2803 | 2817 | CQVLWMEGWS |
| | | | | | | NSSMK |
| 1874.9569 | 1874.863 | −.0939 | −50 | 859 | 874 | EWIVIGQVDME |
| | | | | | | ALVEK |
| 2068.0974 | 2067.8975 | −.1999 | −97 | 1854 | 1870 | ELLTPQQHYD |
| | | | | | | WGLRALK |
| 2081.9858 | 2081.9285 | −.0573 | −28 | 1015 | 1030 | WDKFELMMES |
| | | | | | | HQLMIK |
| 2081.9858 | 2081.9285 | −.0573 | −28 | 1015 | 1030 | WDKFELMMES |
| | | | | | | HQLMIK |
| 2441.0806 | 2441.0813 | .0007 | 0 | 2929 | 2950 | QDEADAALQMI |
| | | | | | | TVSMQD |
| | | | | | | ASEQK |
| 2473.197 | 2473.0913 | −.1057 | −43 | 4130 | 4149 | CPLAWQSKWE |
| | | | | | | GPEDPL |
| | | | | | | QYLR |
| 2473.2258 | 2473.0913 | −.1345 | −54 | 168 | 188 | GILTPSDEFQF |
| | | | | | | WIEQAHR |
| | | | | | | GNK |
| 2501.2671 | 2501.1904 | −.0767 | −31 | 3381 | 3403 | NPNPFIPPDAA |
| | | | | | | SIVTEVNF |
| | | | | | | TTTR |
| 2512.1956 | 2512.2595 | .0639 | 25 | 1018 | 1037 | FELMMESHQL |
| | | | | | | MIKDQIEV MK |

| | |
|---|---|
| hCG2040266 [Homo sapiens] | gi\|119604633 |

Peptide Information

| | | | | Start | End | |
|---|---|---|---|---|---|---|
| Calc. Mass | Obsrv. Mass | ±da | ±ppm | Seq. | Seq. | Sequence |
| 875.3788 | 875.4574 | .0786 | 90 | 104 | 111 | HSRSTACG |
| 1012.5646 | 1012.5323 | −.0323 | −32 | 98 | 106 | LSTVGRHSR |
| 1514.7598 | 1514.6576 | −.1022 | −67 | 6 | 18 | TNQPTWQNPISTK |
| 1857.9454 | 1857.8372 | −.1082 | −58 | 6 | 21 | TNQPTWQNPISTK |
| | | | | | | NTK |
| 2455.1865 | 2455.1028 | −.0837 | −34 | 49 | 68 | VVGCCLLTPKCHT |
| | | | | | | PPLYH |
| | | | | | | MR |

| | |
|---|---|
| TRM1 tRNA methyltransferase 1 homolog | gi\|119604754 |
| (S. cerevisiae), isoform | |
| CRA_d [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 811.3515 | 811.3621 | .0106 | 13 | 42 | 47 | ANCYQR |
| 875.3853 | 875.4574 | .0721 | 82 | 276 | 283 | EDANPSSR |
| 901.456 | 901.4146 | −.0414 | −46 | 215 | 222 | VSLSHACK |
| 1012.5244 | 1012.5323 | .0079 | 8 | 16 | 24 | YGAMALKSR |
| | 1012.5323 | | | | | |
| 1251.7208 | 1251.5962 | −.1246 | −100 | 63 | 73 | VFVRVFTGQAK |
| 1857.9752 | 1857.8372 | −.138 | −74 | 33 | 47 | IVLHSLDLRANCY |
| | | | | | | QR |
| 1874.943 | 1874.863 | −.08 | −43 | 223 | 239 | NAVKTDAPASALW |
| | | | | | | DIMR |
| 1874.943 | 1874.863 | −.08 | −43 | 223 | 239 | NAVKTDAPASALW |
| | | | | | | DIMR |

| | |
|---|---|
| unnamed protein product [Homo sapiens] | gi\|194375826 |

Peptide Information

| | | | | Start | End | |
|---|---|---|---|---|---|---|
| Calc. Mass | Obsrv. Mass | ±da | ±ppm | Seq. | Seq. | Sequence |
| 848.3679 | 848.3872 | .0193 | 23 | 569 | 575 | NCDQVGR |
| 861.3883 | 861.411 | .0227 | 26 | 895 | 901 | NDCSLPR |
| 861.3883 | 861.411 | .0227 | 26 | 895 | 901 | NDCSLPR |
| 875.4468 | 875.4574 | .0106 | 12 | 793 | 800 | LDEVNASK |
| 986.52 | 986.5446 | .0246 | 25 | 963 | 971 | GVGHSMVRK |
| 986.5206 | 986.5446 | .024 | 24 | 1058 | 1064 | TWQWLPR |
| 1003.5101 | 1003.5685 | .0584 | 58 | 319 | 326 | RCLQSPSR |
| 1003.5101 | 1003.5685 | .0584 | 58 | 319 | 326 | RCLQSPSR |
| 1158.5637 | 1158.6351 | .0714 | 62 | 137 | 147 | ENTETPAATPK |
| 1211.6532 | 1211.7167 | .0635 | 52 | 711 | 720 | DTIFYRAAVR |
| 1874.8927 | 1874.863 | −.0297 | −16 | 837 | 854 | HGPSSRGSLTPHP |
| | | | | | | AACDK |
| 1874.8927 | 1874.863 | −.0297 | −16 | 837 | 854 | HGPSSRGSLTPHP |
| | | | | | | AACDK |
| 2096.1362 | 2095.9539 | −.1823 | −87 | 1016 | 1034 | EGMFHHGVPIPVP |
| | | | | | | PLEVLK |
| 2512.3203 | 2512.2595 | −.0608 | −24 | 1013 | 1034 | MPREGMFHHGVPI |
| | | | | | | PVPP |
| | | | | | | LEVLK |

| | |
|---|---|
| bromodomain and PHD finger containing, 1, | gi\|119584376 |
| isoform CRA_a [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 875.4985 | 875.4574 | −.0411 | −47 | 87 | 93 | LPEWYR |
| 986.5206 | 986.5446 | .024 | 24 | 453 | 459 | TWQWLPR |
| 986.5206 | 986.5446 | .024 | 24 | 453 | 459 | TWQWLPR |
| 1158.5637 | 1158.6351 | .0714 | 62 | 48 | 58 | ENTETPAATPK |
| 1211.6532 | 1211.7167 | .0635 | 52 | 257 | 266 | DTIFYRAAVR |
| 1875.9131 | 1875.8636 | −.0495 | −26 | 2 | 17 | SYAQAQRMVEVD |
| | | | | | | LHGR |
| 2096.1362 | 2095.9539 | −.1823 | −87 | 411 | 429 | EGMFHHGVPIPVP |
| | | | | | | PLEVLK |
| 2455.2036 | 2455.1028 | −.1008 | −41 | 206 | 225 | ETVPDYLDHIK |
| | | | | | | KPMDFFT |
| | | | | | | MK |
| 2487.1936 | 2487.0925 | −.1011 | −41 | 206 | 225 | ETVPDYLDHIK |
| | | | | | | KPMDFFT |
| | | | | | | MK |
| 2512.3203 | 2512.2595 | −.0608 | −24 | 408 | 429 | MPREGMFHHG |
| | | | | | | VPIPVPP |
| | | | | | | LEVLK |

| | |
|---|---|
| wingless-type MMTV integration site family, | gi\|119585715 |
| member 5A, isoform CRA_a [Homo sapiens] | |

Protein Group

| | |
|---|---|
| hWNT5A [Homo sapiens] | gi\|348918 |

Peptide Information

| Calc. | | | | Start | End | |
|---|---|---|---|---|---|---|
| Mass | Obsrv. Mass | ±da | ±ppm | Seq. | Seq. | Sequence |
| 806.4189 | 806.3558 | −.0631 | −78 | 112 | 118 | VMQIGSR |
| 815.4734 | 815.3965 | −.0769 | −94 | 269 | 275 | LVQVNSR |
| 901.3754 | 901.4146 | .0392 | 43 | 1 | 9 | MAGSAMSSK |
| 1030.4412 | 1030.5359 | .0947 | 92 | 88 | 94 | ECQYQFR |
| 1030.4412 | 1030.5359 | .0947 | 92 | 88 | 94 | ECQYQFR |
| 1784.7278 | 1784.8611 | .1333 | 75 | 340 | 351 | CHCKFHWCCYVK |
| 2487.1497 | 2487.0925 | −.0572 | −23 | 276 | 296 | FNSPTTQDLVYIDP |
| | | | | | | SPDY |
| | | | | | | CVR |
| 2500.9658 | 2501.1904 | .2246 | 90 | 313 | 333 | TSEGMDGCELMC |
| | | | | | | CGRG |
| | | | | | | YDQFK |

| | | | |
|---|---|---|---|
| | 67/C16 | | Instr./Gel |
| | | | Origin |
| | [1] Sample Project | | Instrument |
| | 20110503 | | Sample |
| | | | Name |
| Protein Name- | KH15 (SEQ ID NO. KH15) | Accession No. | |

| | |
|---|---|
| unnamed protein product [Homo sapiens] | gi\|194391084 |

Peptide Information

| | | | | Start | End | |
|---|---|---|---|---|---|---|
| Calc. Mass | Obsrv. Mass | ±da | ±ppm | Seq. | Seq. | Sequence |
| 861.407 | 861.4113 | .0043 | 5 | 251 | 257 | ICVGCPR |
| 861.407 | 861.4113 | .0043 | 5 | 251 | 257 | ICVGCPR |
| 1003.5684 | 1003.5698 | .0014 | 1 | 176 | 184 | QVVAGLNFR |
| 1003.5684 | 1003.5698 | .0014 | 1 | 176 | 184 | QVVAGLNFR |
| 1030.5356 | 1030.5361 | .0005 | 0 | 305 | 312 | YFIDFVAR |
| 1030.5356 | 1030.5361 | .0005 | 0 | 305 | 312 | YFIDFVAR |
| 1158.6306 | 1158.632 | .0014 | 1 | 304 | 312 | KYFIDFVAR |
| 1158.6306 | 1158.632 | .0014 | 1 | 304 | 312 | KYFIDFVAR |
| 1251.5892 | 1251.5927 | .0035 | 3 | 53 | 63 | TVGSDTFYSFK |
| 1321.653 | 1321.6509 | −.0021 | −2 | 90 | 102 | AATGECTATVG |
| | | | | | | KR |
| 1514.6581 | 1514.6637 | .0056 | 4 | 78 | 89 | TWQDCEYKDA |
| | | | | | | AK |
| 1784.8741 | 1784.8806 | .0065 | 4 | 53 | 67 | TVGSDTFYSFK |
| | | | | | | YEIK |
| 1874.8781 | 1874.8595 | −.0186 | −10 | 32 | 46 | YNSQNQSNNQ |
| | | | | | | FVLYR |
| 1874.8781 | 1874.8595 | −.0186 | −10 | 32 | 46 | YNSQNQSNNQ |
| | | | | | | FVLYR |
| 2002.973 | 2002.9491 | −.0239 | −12 | 31 | 46 | KYNSQNQSNN |
| | | | | | | QFVLYR |
| 2139.0815 | 2139.0762 | −.0053 | −2 | 258 | 276 | DIPTNSPELEET |
| | | | | | | LTHTITK |
| 2441.1038 | 2441.1079 | .0041 | 2 | 208 | 228 | SLWNGDTGEC |
| | | | | | | TDNAYIDI |
| | | | | | | QLR |
| | | | | | | IASFSQNCDIYP |
| 2512.2178 | 2512.2356 | .0178 | 7 | 229 | 250 | GKDFVQ |
| | | | | | | PPTK |
| 2512.2178 | 2512.2356 | .0178 | 7 | 229 | 250 | IASFSQNCDIYPGKDFVQ |

kininogen 1, isoform CRA_a [Homo sapiens]

Protein Group

| | |
|---|---|
| Kininogen 1 [Homo sapiens] | gi\|37748641 |
| PPTK | |
| kininogen 1 variant [Homo sapiens] | gi\|62898910 |
| kininogen 1, isoform CRA_a [Homo sapiens] | gi\|119598587 |
| kininogen 1, isoform CRA_a [Homo sapiens] | |
| kininogen [Homo sapiens] | gi\|386853 |
| kininogen [Homo sapiens] | gi\|28932976 |
| kininogen-1 isoform 2 [Homo sapiens] | gi\|4504893 |
| prepro alpha-2-thiol proteinase inhibitor [Homo sapiens] | gi\|177890 |
| unnamed protein product [Homo sapiens] | gi\|213506121 |
| unnamed protein product [Homo sapiens] | gi\|213506103 |
| unnamed protein product [Homo sapiens] | gi\|207100681 |
| unnamed protein product [Homo sapiens] | gi\|207100663 |
| unnamed protein product [Homo sapiens] | gi\|189054825 |
| unnamed protein product [Homo sapiens] | gi\|158255114 |

Peptide Information

| | | | | Start | | |
|---|---|---|---|---|---|---|
| Calc. Mass | Obsrv. Mass | ±da | ±ppm | Seq. | End Seq. | Sequence |
| 861.407 | 861.4113 | .0043 | 5 | 263 | 269 | ICVGCPR |
| 861.407 | 861.4113 | .0043 | 5 | 263 | 269 | ICVGCPR |
| | 861.4113 | | | | | |
| 1003.5684 | 1003.5698 | .0014 | 1 | 188 | 196 | QVVAGLNFR |
| 1003.5684 | 1003.5698 | .0014 | 1 | 188 | 196 | QVVAGLNFR |
| 1030.5356 | 1030.5361 | .0005 | 0 | 317 | 324 | YFIDFVAR |
| 1030.5356 | 1030.5361 | .0005 | 0 | 317 | 324 | YFIDFVAR |
| 1158.6306 | 1158.632 | .0014 | 1 | 316 | 324 | KYFIDFVAR |
| 1158.6306 | 1158.632 | .0014 | 1 | 316 | 324 | KYFIDFVAR |
| 1251.5892 | 1251.5927 | .0035 | 3 | 65 | 75 | TVGSDTFYSFK |
| 1321.653 | 1321.6509 | −.0021 | −2 | 102 | 114 | AATGECTATVG |
| | | | | | | KR |
| 1514.6581 | 1514.6637 | .0056 | 4 | 90 | 101 | TWQDCEYKDA |
| | | | | | | AK |
| 1784.8741 | 1784.8806 | .0065 | 4 | 65 | 79 | TVGSDTFYSFK |
| | | | | | | YEIK |
| 1874.8781 | 1874.8595 | −.0186 | −10 | 44 | 58 | YNSQNQSNNQ |
| | | | | | | FVLYR |
| 1874.8781 | 1874.8595 | −.0186 | −10 | 44 | 58 | YNSQNQSNNQ |
| | | | | | | FVLYR |
| 2002.973 | 2002.9491 | −.0239 | −12 | 43 | 58 | KYNSQNQSNN |
| | | | | | | QFVLYR |
| 2139.0815 | 2139.0762 | −.0053 | −2 | 270 | 288 | DIPTNSPELEET |
| | | | | | | LTHTITK |
| 2441.1038 | 2441.1079 | .0041 | 2 | 220 | 240 | SLWNGDTGEC |
| | | | | | | TDNAYIDI |
| | | | | | | QLR |
| 2512.2178 | 2512.2356 | .0178 | 7 | 241 | 262 | IASFSQNCDIYP |
| | | | | | | GKDFVQ |
| | | | | | | PPTK |
| 2512.2178 | 2512.2356 | .0178 | 7 | 241 | 262 | IASFSQNCDIYP |
| | | | | | | GKDFVQ |
| | | | | | | PPTK |

| | |
|---|---|
| RecName: Full = Kininogen-1; AltName: | gi\|124056474 |

Protein Group

| | |
|---|---|
| Full = Alpha-2-thiol proteinase | |
| inhibitor; AltName: Full = Fitzgerald | |
| factor; AltName: Full = High | |
| molecular | |
| weight kininogen; | |
| Short = HMWK; | |
| AltName: | |
| Full = Williams-Fitzgerald-Flaujeac factor; Contains: | |
| RecName: Full = Kininogen-1 heavy | |
| chain; Contains: RecName: | |
| Full = T-kinin; AltName: | |
| Full = Ile-Ser- | |
| Bradykinin; Contains: | |
| RecName: | |
| Full = Bradykinin; AltName: Full = Kallidin I; Contains: | |
| RecName: Full = Lysyl- | |
| bradykinin; AltName: | |
| Full = Kallidin | |
| II; Contains: RecName: Full = Kininogen-1 | |
| light chain; Contains: RecName: Full = Low molecular | |
| weight growth-promoting factor; Flags: Precursor | |
| kininogen 1, isoform CRA_b [Homo sapiens] | gi\|119598586 |
| kininogen | gi\|386852 |
| [Homo sapiens] | |
| kininogen-1 isoform 1 [Homo sapiens] | gi\|156231037 |
| unnamed protein product [Homo sapiens] | gi\|298886629 |
| unnamed protein product [Homo sapiens] | gi\|194383526 |

Peptide Information

| | | | | Start | End | |
|---|---|---|---|---|---|---|
| Calc. Mass | Obsrv. Mass | ±da | ±ppm | Seq. | Seq. | Sequence |
| 861.407 | 861.4113 | .0043 | 5 | 263 | 269 | ICVGCPR |
| 861.407 | 861.4113 | .0043 | 5 | 263 | 269 | ICVGCPR |
| 1003.5684 | 1003.5698 | .0014 | 1 | 188 | 196 | QVVAGLNFR |
| 1003.5684 | 1003.5698 | .0014 | 1 | 188 | 196 | QVVAGLNFR |
| 1030.5356 | 1030.5361 | .0005 | 0 | 317 | 324 | YFIDFVAR |
| 1030.5356 | 1030.5361 | .0005 | 0 | 317 | 324 | YFIDFVAR |
| 1158.6306 | 1158.632 | .0014 | 1 | 316 | 324 | KYFIDFVAR |
| 1158.6306 | 1158.632 | .0014 | 1 | 316 | 324 | KYFIDFVAR |
| 1251.5892 | 1251.5927 | .0035 | 3 | 65 | 75 | TVGSDTFYSFK |
| 1321.653 | 1321.6509 | −.0021 | −2 | 102 | 114 | AATGECTATVGKR |
| | 1321.6509 | | | | | |
| 1514.6581 | 1514.6637 | .0056 | 4 | 90 | 101 | TWQDCEYKDAAK |
| 1784.8741 | 1784.8806 | .0065 | 4 | 65 | 79 | TVGSDTFYSFKYEIK |
| 1874.8781 | 1874.8595 | −.0186 | −10 | 44 | 58 | YNSQNQSNNQFVLYR |
| 1874.8781 | 1874.8595 | −.0186 | −10 | 44 | 58 | YNSQNQSNNQFVLYR |
| 2002.973 | 2002.9491 | −.0239 | −12 | 43 | 58 | KYNSQNQSNNQFVLYR |
| 2139.0815 | 2139.0762 | −.0053 | −2 | 270 | 288 | DIPTNSPELEETLTHTITK |
| 2441.1038 | 2441.1079 | .0041 | 2 | 220 | 240 | SLWNGDTGE |
| | | | | | | CTDNAYIDI QLR |
| 2512.2178 | 2512.23 | .0178 | 7 | 24 | 26 | IASFSQNCDIY |
| | | | | | | PPTK |
| 2512.2178 | 2512.23 | .0178 | 7 | 24 | 26 | IASFSQNCDIY |
| | 56 | | | 1 | 2 | PGKDFVO |

| | |
|---|---|
| kininogen-1 isoform 3 [Homo sapiens] | gi\|262050546 |

Peptide Information

| | | | | Start | End | |
|---|---|---|---|---|---|---|
| Calc. Mass | Obsrv. Mass | ±da | ±ppm | Seq. | Seq. | Sequence |
| 861.407 | 861.4113 | .0043 | 5 | 227 | 233 | ICVGCPR |
| 861.407 | 861.4113 | .0043 | 5 | 227 | 233 | ICVGCPR |
| 1030.5356 | 1030.5361 | .0005 | 0 | 281 | 288 | YFIDFVAR |
| 1030.5356 | 1030.5361 | .0005 | 0 | 281 | 288 | YFIDFVAR |
| 1158.6306 | 1158.632 | .0014 | 1 | 280 | 288 | KYFIDFVAR |
| 1158.6306 | 1158.632 | .0014 | 1 | 280 | 288 | KYFIDFVAR |
| 1251.5892 | 1251.5927 | .0035 | 3 | 65 | 75 | TVGSDTFYSFK |
| 1321.653 | 1321.6509 | −.0021 | −2 | 102 | 114 | AATGECTATVGKR |
| 1514.6581 | 1514.6637 | .0056 | 4 | 90 | 101 | TWQDCEYKDAAK |
| 1784.8741 | 1784.8806 | .0065 | 4 | 65 | 79 | TVGSDTFYSFKYEIK |
| 1874.8781 | 1874.8595 | −.0186 | −10 | 44 | 58 | YNSQNQSNNQFV |
| | | | | | | LYR |
| 1874.8781 | 1874.8595 | −.0186 | −10 | 44 | 58 | YNSQNQSNNQFV |
| | | | | | | LYR |
| 2002.973 | 2002.9491 | −.0239 | −12 | 43 | 58 | KYNSQNQSNNQF |
| | | | | | | VLYR |
| 2139.0815 | 2139.0762 | −.0053 | −2 | 234 | 252 | DIPTNSPELEETLT |
| | | | | | | HTITK |
| 2512.2178 | 2512.2356 | .0178 | 7 | 205 | 226 | IASFSQNCDIYPGK |
| | | | | | | DFVQ |
| | | | | | | PPTK |
| 2512.2178 | 2512.2356 | .0178 | 7 | 205 | 226 | IASFSQNCDIYPGK |
| | | | | | | DFVQ |
| | | | | | | PPTK |

| | |
|---|---|
| KNG1 protein [Homo sapiens] | gi\|116283732 |

Peptide Information

| | | | | Start | | |
|---|---|---|---|---|---|---|
| Calc. Mass | Obsrv. Mass | ±da | ±ppm | Seq. | End Seq. | Sequence |
| 861.407 | 861.4113 | .0043 | 5 | 127 | 133 | ICVGCPR |
| | 861.4113 | | | | | |
| 861.407 | 861.4113 | .0043 | 5 | 127 | 133 | ICVGCPR |
| 1003.5684 | 1003.5698 | .0014 | 1 | 52 | 60 | QVVAGLNFR |
| 1003.5684 | 1003.5698 | .0014 | 1 | 52 | 60 | QVVAGLNFR |
| 1030.5356 | 1030.5361 | .0005 | 0 | 181 | 188 | YFIDFVAR |
| 1030.5356 | 1030.5361 | .0005 | 0 | 181 | 188 | YFIDFVAR |
| 1158.6306 | 1158.632 | .0014 | 1 | 180 | 188 | KYFIDFVAR |
| 1158.6306 | 1158.632 | .0014 | 1 | 180 | 188 | KYFIDFVAR |
| 2139.0815 | 2139.0762 | −.0053 | −2 | 134 | 152 | DIPTNSPELEET |
| | | | | | | LTHTITK |
| 2441.1038 | 2441.1079 | .0041 | 2 | 84 | 104 | SLWNGDTGEC |
| | | | | | | TDNAYIDI |
| | | | | | | QLR |
| 2512.2178 | 2512.2356 | .0178 | 7 | 105 | 126 | IASFSQNCDIYP |
| | | | | | | GKDFVQ |
| | | | | | | PPTK |
| 2512.2178 | 2512.2356 | .0178 | 7 | 105 | 126 | IASFSQNCDIY |
| | | | | | | PGKDFVQ |
| | | | | | | PPTK |

| | |
|---|---|
| hCG2032408, isoform CRA_a [Homo sapiens] | gi\|119587443 |

Peptide Information

| | Obsrv. | | | Start | End | |
|---|---|---|---|---|---|---|
| Calc. Mass | Mass | ±da | ±ppm | Seq. | Seq. | Sequence |
| 801.4213 | 801.3756 | −.0457 | −57 | 678 | 684 | LQNAAER |
| 803.4006 | 803.3851 | −.0155 | −19 | 2 | 9 | ANGTADVR |
| 805.4162 | 805.3634 | −.0528 | −66 | 652 | 660 | NSKAGSGGK |
| 807.428 | 807.3718 | −.0562 | −70 | 1377 | 1383 | SIMTDIK |
| 815.4522 | 815.4123 | −.0399 | −49 | 2589 | 2595 | GLIHYGR |
| 818.4254 | 818.3859 | −.0395 | −48 | 1183 | 1189 | LQSEVDK |
| 826.3876 | 826.3891 | .0015 | 2 | 3501 | 3506 | INNMYR |
| 827.4443 | 827.395 | −.0493 | −60 | 2983 | 2989 | MPPDVIR |
| 844.4774 | 844.4187 | −.0587 | −70 | 522 | 528 | DLLDQLK |
| 848.4658 | 848.4327 | −.0331 | −39 | 1976 | 1983 | AALCKTGK |
| 856.4635 | 856.4633 | −.0002 | 0 | 3282 | 3288 | THLKDSR |
| 861.4862 | 861.4113 | −.0749 | −87 | 1307 | 1313 | CLLQSLK |
| 861.4862 | 861.4113 | −.0749 | −87 | 1307 | 1313 | CLLQSLK |
| 873.4689 | 873.3929 | −.076 | −87 | 714 | 719 | QQQRWK |
| 1002.5102 | 1002.5387 | .0285 | 28 | 3411 | 3418 | LLQQEEDK |
| 1003.524 | 1003.5698 | .0458 | 46 | 2057 | 2065 | LLTMPSGER |
| 1003.524 | 1003.5698 | .0458 | 46 | 2057 | 2065 | LLTMPSGER |
| 1158.5824 | 1158.632 | .0496 | 43 | 1173 | 1182 | VEEHSVMTVK |
| 1158.5824 | 1158.632 | .0496 | 43 | 1173 | 1182 | VEEHSVMTVK |
| 1183.6616 | 1183.649 | −.0126 | −11 | 2980 | 2989 | SLRMPPDVIR |
| | 1183.649 | | | | | |
| 1514.7383 | 1514.6637 | −.0746 | −49 | 2998 | 3010 | LMGIFDTSWVSMK |
| 1546.728 | 1546.6477 | −.0803 | −52 | 2998 | 3010 | LMGIFDTSWVSMK |
| 1546.728 | 1546.6477 | −.0803 | −52 | 2998 | 3010 | LMGIFDTSWVSMK |
| 1857.9963 | 1857.8433 | −.153 | −82 | 1399 | 1413 | NSLLTILDQLQRCQR |
| 1874.787 | 1874.8595 | .0725 | 39 | 2778 | 2792 | CQVLWMEGWSNSSMK |
| 1874.9569 | 1874.8595 | −.0974 | −52 | 859 | 874 | EWIVIGQVDMEALVEK |
| 2002.882 | 2002.9491 | .0671 | 34 | 2778 | 2793 | CQVLWMEGWSNSSMKK |
| 2068.0974 | 2067.9004 | −.197 | −95 | 1842 | 1858 | ELLTPQQHYDWGLRALK |
| 2081.9858 | 2081.9248 | −.061 | −29 | 1015 | 1030 | WDKFELMMESHQLMIK |
| 2441.0806 | 2441.1079 | .0273 | 11 | 2904 | 2925 | QDEADAALQ |
| | | | | | | MITVSMQD |
| | | | | | | ASEQK |
| 2473.2258 | 2473.0701 | −.1557 | −63 | 168 | 188 | GILTPSDEFQF |
| | | | | | | WIEQAHR GNK |
| 2473.2258 | 2473.0701 | −.1557 | −63 | 168 | 188 | GILTPSDEFQF |
| | | | | | | WIEQAHR GNK |
| 2512.1956 | 2512.2356 | .04 | 16 | 1018 | 1037 | FELMMESHQL |
| | | | | | | MIKDQIEV MK |
| 2512.1956 | 2512.235 | .0 | 1 | 10 | 103 | FELMMESHQL |
| | | | | | | MK |

| | |
|---|---|
| unnamed protein product [Homo sapiens] | gi\|40976466 |

Peptide Information

| | | | | Start | | |
|---|---|---|---|---|---|---|
| Calc. Mass | Obsrv. Mass | ±da | ±ppm | Seq. | End Seq. | Sequence |
| 818.4553 | 818.3859 | −.0694 | −85 | 13 | 18 | LLERCK |
| 824.376 | 824.4156 | .0396 | 48 | 19 | 24 | YWPACK |
| 1784.7479 | 1784.8806 | .1327 | 74 | 25 | 39 | NGDECAYHHPISP |
| | | | | | | CK |
| 1858.0103 | 1857.8433 | −.167 | −90 | 1 | 16 | MSELSVAQKPEKL |
| | | | | | | LER |
| 2081.9531 | 2081.9248 | −.0283 | −14 | 59 | 75 | YDAKCTKPDCPFT |
| | | | | | | HVSR |

| | |
|---|---|
| homeobox protein [Homo sapiens] | gi\|7414351 |

Peptide Information

| | | | | Start | End | |
|---|---|---|---|---|---|---|
| Calc. Mass | Obsrv. Mass | ±da | ±ppm | Seq. | Seq. | Sequence |
| 800.3897 | 800.3841 | −.0056 | −7 | 43 | 50 | APSESGPR |
| 824.4083 | 824.4156 | .0073 | 9 | 426 | 431 | RCPYTK |
| 827.437 | 827.395 | −.042 | −51 | 458 | 464 | SVHLTDR |
| 844.4635 | 844.4187 | −.0448 | −53 | 353 | 359 | SEPSIRR |
| 863.4522 | 863.4113 | −.0409 | −47 | 468 | 473 | IWFQNR |
| 922.5217 | 922.4904 | −.0313 | −34 | 120 | 127 | LAHQLGQR |
| 1002.6207 | 1002.5387 | −.082 | −82 | 81 | 88 | IQHVLHKK |
| | 1002.5387 | | | | | |
| 1003.5009 | 1003.5698 | .0689 | 69 | 133 | 139 | WWWRGGR |
| 1003.5009 | 1003.5698 | .0689 | 69 | 133 | 139 | WWWRGGR |
| 1875.9196 | 1875.8578 | −.0618 | −33 | 403 | 420 | DSLGNSK |
| | | | | | | GENAANWLTAK |
| 2002.9805 | 2002.9491 | −.0314 | −16 | 1 | 20 | MPGPPPA |
| | | | | | | TLGSPHSSPF |
| | | | | | | GPR |
| 2082.0959 | 2081.9248 | −.1711 | −82 | 301 | 318 | LLRPTRPPRAMAAGGCWR |

| | |
|---|---|
| Br140 [Homo sapiens] | gi\|190352 |

Peptide Information

| | | | | Start | | |
|---|---|---|---|---|---|---|
| Calc. Mass | Obsrv. Mass | ±da | ±ppm | Seq. | End Seq. | Sequence |
| 800.4373 | 800.3841 | −.0532 | −66 | 67 | 73 | QSRPANK |
| 814.4094 | 814.4108 | .0014 | 2 | 711 | 716 | DTIFYR |
| 815.437 | 815.4123 | −.0247 | −30 | 723 | 730 | EQGGAVVR |
| 848.4658 | 848.4327 | −.0331 | −39 | 814 | 820 | KEMTALR |
| 861.3883 | 861.4113 | .023 | 27 | 990 | 996 | NDCSLPR |
| 861.3883 | 861.4113 | .023 | 27 | 990 | 996 | NDCSLPR |
| 986.52 | 986.5446 | .0246 | 25 | 1058 | 1066 | GVGHSMVRK |
| 986.5206 | 986.5446 | .024 | 24 | 1153 | 1159 | TWQWLPR |
| 1003.5101 | 1003.5698 | .0597 | 59 | 319 | 326 | RCLQSPSR |
| 1003.5101 | 1003.5698 | .0597 | 59 | 319 | 326 | RCLQSPSR |
| 1158.5637 | 1158.632 | .0683 | 59 | 137 | 147 | ENTETPAATPK |
| 1158.5637 | 1158.632 | .0683 | 59 | 137 | 147 | ENTETPAATPK |
| 1211.6532 | 1211.7495 | .0963 | 79 | 711 | 720 | DTIFYRAAVR |
| 1874.8927 | 1874.8595 | −.0332 | −18 | 837 | 854 | HGPSSRGSLTP |
| | | | | | | HPAACDK |
| 1874.8927 | 1874.8595 | −.0332 | −18 | 837 | 854 | HGPSSRGSLTP |
| | | | | | | HPAACDK |
| 2096.1362 | 2095.9319 | −.2043 | −97 | 1111 | 1129 | EGMFHHGVPIP |
| | | | | | | VPPLEVLK |
| 2512.3203 | 2512.2356 | −.0847 | −34 | 1108 | 1129 | MPREGMFHHG |
| | | | | | | VPIPVPP |
| | | | | | | LEVLK |
| 2512.3203 | 2512.2356 | −.0847 | −34 | 1108 | 1129 | MPREGMFHHG |
| | | | | | | VPIPVPP |
| | | | | | | LEVLK |

| | |
|---|---|
| coiled-coil alpha-helical rod protein 1 | gi\|168985836 |
| [Homo sapiens] | |

Protein Group

| | |
|---|---|
| coiled-coil alpha-helical rod protein 1 [Homo sapiens] | gi\|168985398 |
| coiled-coil alpha-helical rod protein 1 [Homo sapiens] | |
| coiled-coil alpha-helical rod protein 1 [Homo sapiens] | i\|168984995 |
| coiled-coil alpha-helical rod protein 1 [Homo sapiens] | gi\|123287162 |
| coiled-coil alpha-helical rod protein 1 [Homo sapiens] | gi\|220860570 |

Peptide Information

| | | | | Start | | |
|---|---|---|---|---|---|---|
| Calc. Mass | Obsrv. Mass | ±da | ±ppm | Seq. | End Seq. | Sequence |
| 803.4257 | 803.3851 | −.0406 | −51 | 92 | 98 | VAEVETR |
| 818.3638 | 818.3859 | .0221 | 27 | 22 | 28 | EQGEAER |
| 873.4788 | 873.3929 | −.0859 | −98 | 144 | 150 | LQEEARK |
| 1030.5527 | 1030.5361 | −.0166 | −16 | 160 | 167 | LQELERDK |
| 1030.5527 | 1030.5361 | −.0166 | −16 | 160 | 167 | LQELERDK |
| 1211.761 | 1211.7495 | −.0115 | −9 | 174 | 184 | LLTVLPSLLDK |
| 2003.0543 | 2002.9491 | −.1052 | −53 | 215 | 233 | GSLSVLLDDLQDL |
| | | | | | | SEAISK |
| 2486.9817 | 2487.0879 | .1062 | 43 | 234 | 255 | EEAVCQGDNLDR |
| | | | | | | CSSSN |
| | | | | | | PQMSS |

| | |
|---|---|
| KIAA0615 protein [Homo sapiens] | gi\|40788302 |

Peptide Information

| | | | | Start | | |
|---|---|---|---|---|---|---|
| Calc. Mass | Obsrv. Mass | ±da | ±ppm | Seq. | End Seq. | Sequence |
| 814.4669 | 814.4108 | −.0561 | −69 | 512 | 518 | TVPIEQK |
| 839.4118 | 839.3781 | −.0337 | −40 | 557 | 564 | GASSHQPR |
| 844.4134 | 844.4187 | .0053 | 6 | 682 | 687 | KFFSCR |
| 986.5417 | 986.5446 | .0029 | 3 | 179 | 186 | SHIQQFVK |
| 986.5417 | 986.5446 | .0029 | 3 | 179 | 186 | SHIQQFVK |
| 1002.5466 | 1002.5387 | −.0079 | −8 | 287 | 296 | AGTPVSELTK |
| 1003.6186 | 1003.5698 | −.0488 | −49 | 649 | 656 | IPYKLELK |
| 1003.6186 | 1003.5698 | −.0488 | −49 | 649 | 656 | IPYKLELK |
| 1158.6226 | 1158.632 | .0094 | 8 | 64 | 73 | AELLEQSRGR |
| 1158.6226 | 1158.632 | .0094 | 8 | 64 | 73 | AELLEQSRGR |
| 1321.5988 | 1321.6509 | .0521 | 39 | 496 | 506 | VEAKPCTSNCR |
| 1514.7122 | 1514.6637 | −.0485 | −32 | 14 | 28 | EGPTPPDWLGSS |
| | | | | | | GSK |
| 1784.8563 | 1784.8806 | .0243 | 14 | 11 | 28 | GGREGPTPPDWL |
| | | | | | | GSSG |
| | | | | | | SK |
| 2512.3018 | 2512.2356 | −.0662 | −26 | 565 | 586 | VPLFPENGLHQQP |
| | | | | | | EPLLP |
| | | | | | | NNMK |
| 2512.3018 | 2512.2356 | −.0662 | −26 | 565 | 586 | VPLFPENGLHQQP |
| | | | | | | EPLLP |
| | | | | | | NNMK |

| | |
|---|---|
| DYNC2H1 variant protein [Homo sapiens] | gi\|166788546 |

Peptide Information

| | | | | Start | | |
|---|---|---|---|---|---|---|
| Calc. Mass | Obsrv. Mass | ±da | ±ppm | Seq. | End Seq. | Sequence |
| 801.4213 | 801.3756 | −.0457 | −57 | 678 | 684 | LQNAAER |
| 803.4006 | 803.3851 | −.0155 | −19 | 2 | 9 | ANGTADVR |
| 804.421 | 804.384 | −.037 | −46 | 3603 | 3609 | KADSQQK |
| 805.4162 | 805.3634 | −.0528 | −66 | 652 | 660 | NSKAGSGGK |
| 807.428 | 807.3718 | −.0562 | −70 | 1377 | 1383 | SIMTDIK |
| 815.4522 | 815.4123 | −.0399 | −49 | 2614 | 2620 | GLIHYGR |
| 818.4254 | 818.3859 | −.0395 | −48 | 1183 | 1189 | LQSEVDK |
| 826.3876 | 826.3891 | .0015 | 2 | 3512 | 3517 | INNMYR |
| 827.4443 | 827.395 | −.0493 | −60 | 3008 | 3014 | MPPDVIR |
| 844.4774 | 844.4187 | −.0587 | −70 | 522 | 528 | DLLDQLK |
| 848.4658 | 848.4327 | −.0331 | −39 | 1994 | 2001 | AALCKTGK |
| 856.4635 | 856.4633 | −.0002 | 0 | 3293 | 3299 | THLKDSR |
| 861.4862 | 861.4113 | −.0749 | −87 | 1307 | 1313 | CLLQSLK |
| 861.4862 | 861.4113 | −.0749 | −87 | 1307 | 1313 | CLLQSLK |
| | 861.4113 | | | | | |
| 873.4689 | 873.3929 | −.076 | −87 | 714 | 719 | QQQRWK |
| 1002.5102 | 1002.5387 | .0285 | 28 | 3422 | 3429 | LLQQEEDK |
| 1003.524 | 1003.5698 | .0458 | 46 | 2075 | 2083 | LLTMPSGER |
| 1003.524 | 1003.5698 | .0458 | 46 | 2075 | 2083 | LLTMPSGER |
| 1158.5824 | 1158.632 | .0496 | 43 | 1173 | 1182 | VEEHSVMTVK |
| 1158.5824 | 1158.632 | .0496 | 43 | 1173 | 1182 | VEEHSVMTVK |
| 1183.6616 | 1183.649 | −.0126 | −11 | 3005 | 3014 | SLRMPPDVIR |
| 1514.7383 | 1514.6637 | −.0746 | −49 | 3023 | 3035 | LMGIFDTSWVS |
| | | | | | | MK |
| 1546.728 | 1546.6477 | −.0803 | −52 | 3023 | 3035 | LMGIFDTSWVS |
| | | | | | | MK |
| 1546.728 | 1546.6477 | −.0803 | −52 | 3023 | 3035 | LMGIFDTSWVS |
| | | | | | | MK |
| 1857.9963 | 1857.8433 | −.153 | −82 | 1399 | 1413 | NSLLTILDQLQ |
| | | | | | | RCQR |
| 1874.787 | 1874.8595 | .0725 | 39 | 2803 | 2817 | CQVLWMEGW |
| | | | | | | SNSSMK |
| 1874.9569 | 1874.8595 | −.0974 | −52 | 859 | 874 | EWIVIGQVDME |
| | | | | | | ALVEK |
| 2002.882 | 2002.9491 | .0671 | 34 | 2803 | 2818 | CQVLWMEGW |
| | | | | | | SNSSMKK |
| 2068.0974 | 2067.9004 | −.197 | −95 | 1854 | 1870 | ELLTPQQHYD |
| | | | | | | WGLRALK |
| 2081.9858 | 2081.9248 | −.061 | −29 | 1015 | 1030 | WDKFELMMES |
| | | | | | | HQLMIK |
| 2441.0806 | 2441.1079 | .0273 | 11 | 2929 | 2950 | QDEADAALQMI |
| | | | | | | TVSMQD |
| | | | | | | ASEQK |
| 2473.197 | 2473.0701 | −.1269 | −51 | 4123 | 4142 | CPLAWQSKWE |
| | | | | | | GPEDPL |
| | | | | | | QYLR |
| 2473.2258 | 2473.0701 | −.1557 | −63 | 168 | 188 | GILTPSDEFQF |
| | | | | | | WIEQAHR |
| | | | | | | GNK |
| 2512.1956 | 2512.2356 | .04 | 16 | 1018 | 1037 | FELMMESHQL |
| | | | | | | MIKDQIEV |
| | | | | | | MK |
| 2512.1956 | 2512.2356 | .04 | 16 | 1018 | 1037 | FELMMESHQL |
| | | | | | | MIKDQIEV MK |

| | |
|---|---|
| unnamed protein product [Homo sapiens] | gi\|194375826 |

Peptide Information

| | | | | Start | End | |
|---|---|---|---|---|---|---|
| Calc. Mass | Obsrv. Mass | ±da | ±ppm | Seq. | Seq. | Sequence |
| 800.4373 | 800.3841 | −.0532 | −66 | 67 | 73 | QSRPANK |
| 814.4094 | 814.4108 | .0014 | 2 | 711 | 716 | DTIFYR |
| 848.4658 | 848.4327 | −.0331 | −39 | 814 | 820 | KEMTALR |
| 861.3883 | 861.4113 | .023 | 27 | 895 | 901 | NDCSLPR |
| 861.3883 | 861.4113 | .023 | 27 | 895 | 901 | NDCSLPR |
| 986.52 | 986.5446 | .0246 | 25 | 963 | 971 | GVGHSMVRK |
| 986.5206 | 986.5446 | .024 | 24 | 1058 | 1064 | TWQWLPR |
| 1003.5101 | 1003.5698 | .0597 | 59 | 319 | 326 | RCLQSPSR |
| 1003.5101 | 1003.5698 | .0597 | 59 | 319 | 326 | RCLQSPSR |
| 1158.5637 | 1158.632 | .0683 | 59 | 137 | 147 | ENTETPAATPK |
| 1158.5637 | 1158.632 | .0683 | 59 | 137 | 147 | ENTETPAATPK |
| 1211.6532 | 1211.7495 | .0963 | 79 | 711 | 720 | DTIFYRAAVR |
| 1874.8927 | 1874.8595 | −.0332 | −18 | 837 | 854 | HGPSSRGSLT |
| | | | | | | PHPAACDK |
| 1874.8927 | 1874.8595 | −.0332 | −18 | 837 | 854 | HGPSSRGSLT |
| | | | | | | PHPAACDK |
| 2096.1362 | 2095.9319 | −.2043 | −97 | 1016 | 1034 | EGMFHHGVPIP |
| | | | | | | VPPLEVLK |
| 2512.3203 | 2512.2356 | −.0847 | −34 | 1013 | 1034 | MPREGMFHHG |
| | | | | | | VPIPVPP |
| | | | | | | LEVLK |
| 2512.3203 | 2512.2356 | −.0847 | −34 | 1013 | 1034 | MPREGMFHHG |
| | | | | | | VPIPVPP |
| | | | | | | LEVLK |

| | |
|---|---|
| RREB1 protein [Homo sapiens] | gi\|124297065 |

Peptide Information

| | | | | Start | End | |
|---|---|---|---|---|---|---|
| Calc. Mass | Obsrv. Mass | ±da | ±ppm | Seq. | Seq. | Sequence |
| 805.4414 | 805.3634 | −.078 | −97 | 293 | 300 | SVASKADK |
| 814.4431 | 814.4108 | −.0323 | −40 | 358 | 364 | HAKHHGK |
| 853.3291 | 853.3855 | .0564 | 66 | 333 | 338 | CQTCER |
| 856.4886 | 856.4633 | −.0253 | −30 | 43 | 50 | NGLIPQSK |
| 873.4312 | 873.3929 | −.0383 | −44 | 209 | 215 | QEPKDEK |
| 922.4853 | 922.4904 | .0051 | 6 | 205 | 212 | AHGRQEPK |
| 1030.5099 | 1030.5361 | .0262 | 25 | 33 | 41 | HGVTTCSLR |
| 1030.5099 | 1030.5361 | .0262 | 25 | 33 | 41 | HGVTTCSLR |
| 1158.6049 | 1158.632 | .0271 | 23 | 32 | 41 | KHGVTTCSLR |
| 1158.6049 | 1158.632 | .0271 | 23 | 32 | 41 | KHGVTTCSLR |
| 1321.7012 | 1321.6509 | −.0503 | −38 | 311 | 320 | RFWSLQDLTR |
| 1514.7281 | 1514.6637 | −.0644 | −43 | 321 | 332 | HMRSHTGERPYK |

| | |
|---|---|
| hCG2032408, isoform CRA_e [Homo sapiens] | gi\|119587447 |

Peptide Information

| | Obsrv. | | | Start | End | |
|---|---|---|---|---|---|---|
| Calc. Mass | Mass | ±da | ±ppm | Seq. | Seq. | Sequence |
| 801.4213 | 801.3756 | −.0457 | −57 | 678 | 684 | LQNAAER |
| 803.4006 | 803.3851 | −.0155 | −19 | 2 | 9 | ANGTADVR |
| 805.4162 | 805.3634 | −.0528 | −66 | 652 | 660 | NSKAGSGGK |
| 807.428 | 807.3718 | −.0562 | −70 | 1377 | 1383 | SIMTDIK |
| 815.4522 | 815.4123 | −.0399 | −49 | 2589 | 2595 | GLIHYGR |
| 818.4254 | 818.3859 | −.0395 | −48 | 1183 | 1189 | LQSEVDK |
| 826.3876 | 826.3891 | .0015 | 2 | 3497 | 3502 | INNMYR |
| 827.4443 | 827.395 | −.0493 | −60 | 2983 | 2989 | MPPDVIR |
| 844.4774 | 844.4187 | −.0587 | −70 | 522 | 528 | DLLDQLK |
| 848.4658 | 848.4327 | −.0331 | −39 | 1976 | 1983 | AALCKT |
| | | | | | | GK |
| 856.4635 | 856.4633 | −.0002 | 0 | 3278 | 3284 | THLKDSR |
| 861.4862 | 861.4113 | −.0749 | −87 | 1307 | 1313 | CLLQSLK |
| 861.4862 | 861.4113 | −.0749 | −87 | 1307 | 1313 | CLLQSLK |
| 873.4689 | 873.3929 | −.076 | −87 | 714 | 719 | QQQRWK |
| 1002.5102 | 1002.5387 | .0285 | 28 | 3407 | 3414 | LLQQEE |
| | | | | | | DK |
| 1003.524 | 1003.5698 | .0458 | 46 | 2057 | 2065 | LLTMPS |
| | | | | | | GER |
| 1003.524 | 1003.5698 | .0458 | 46 | 2057 | 2065 | LLTMPSGER |
| 1158.5824 | 1158.632 | .0496 | 43 | 1173 | 1182 | VEEHSVMTVK |
| 1158.5824 | 1158.632 | .0496 | 43 | 1173 | 1182 | VEEHSVMTVK |
| 1183.6616 | 1183.649 | −.0126 | −11 | 2980 | 2989 | SLRMPPDVIR |
| 1514.7383 | 1514.6637 | −.0746 | −49 | 2998 | 3010 | LMGIFDTSWVS |
| | | | | | | MK |
| 1546.728 | 1546.6477 | −.0803 | −52 | 2998 | 3010 | LMGIFDTSWVS |
| | | | | | | MK |
| 1546.728 | 1546.6477 | −.0803 | −52 | 2998 | 3010 | LMGIFDTSWVS |
| | | | | | | MK |
| 1857.9963 | 1857.8433 | −.153 | −82 | 1399 | 1413 | NSLLTILDQLQ |
| | | | | | | RCQR |
| 1874.787 | 1874.8595 | .0725 | 39 | 2778 | 2792 | CQVLWMEGW |
| | | | | | | SNSSMK |
| 1874.9569 | 1874.8595 | −.0974 | −52 | 859 | 874 | EWIVIGQVDME |
| | | | | | | ALVEK |
| 2002.882 | 2002.9491 | .0671 | 34 | 2778 | 2793 | CQVLWMEGW |
| | | | | | | SNSSMKK |
| 2068.0974 | 2067.9004 | −.197 | −95 | 1842 | 1858 | ELLTPQQHYD |
| | | | | | | WGLRALK |
| 2081.9858 | 2081.9248 | −.061 | −29 | 1015 | 1030 | WDKFELMMES |
| | | | | | | HQLMIK |
| 2441.0806 | 2441.1079 | .0273 | 11 | 2904 | 2925 | QDEADAALQMI |
| | | | | | | TVSMQD |
| | | | | | | ASEQK |
| 2473.197 | 2473.0701 | −.1269 | −51 | 4091 | 4110 | CPLAWQSKWE |
| | | | | | | GPEDPL |
| | | | | | | QYLR |
| 2473.2258 | 2473.0701 | −.1557 | −63 | 168 | 188 | GILTPSDEFQF |
| | | | | | | WIEQAHR |
| | | | | | | GNK |
| 2512.1956 | 2512.2356 | .04 | 16 | 1018 | 1037 | FELMMESHQL |
| | | | | | | MIKDQIEV |
| | | | | | | MK |
| 2512.1956 | 2512.2356 | .04 | 16 | 1018 | 1037 | FELMMESHQL |
| | | | | | | MIKDQIEV |
| | | | | | | MK |

| | |
|---|---|
| RecName: Full = Uncharacterized protein | gi\|156630925 |
| C9orf24; | |
| AltName: Full = Testis development protein | |
| NYD-SP22 | |

Protein Group

| | |
|---|---|
| Chromosome 9 open reading frame 24 [Homo sapiens] | gi\|20809872 |
| Chromosome 9 open reading frame 24 [Homo sapiens] | |
| Chromosome 9 open reading frame 24 [Homo sapiens] | gi\|18314508 |
| chromosome 9 open reading frame 24 [Homo sapiens] | gi\|55958908 |
| chromosome 9 open reading frame 24, isoform | gi\|119578860 |
| CRA_a [Homo sapiens] | |
| testes development-related NYD-SP22 isoform 1 [Homo | gi\|21362074 |
| sapiens] | |
| unnamed protein product [Homo sapiens] | gi\|16554112 |

Peptide Information

| | Obsrv. | | | Start | End | |
|---|---|---|---|---|---|---|
| Calc. Mass | Mass | ±da | ±ppm | Seq. | Seq. | Sequence |
| 800.4124 | 800.3841 | −.0283 | −35 | 1 | 6 | MFLFSR |
| 1175.515 | 1175.4698 | −.0452 | −38 | 66 | 74 | CAVQDYTYR |
| 1251.7096 | 1251.5927 | −.1169 | −93 | 123 | 132 | YTTYLPRLPK |
| 1546.7496 | 1546.6477 | −.1019 | −66 | 8 | 20 | TRTPISTYSDSYR |
| 1546.7496 | 1546.6477 | −.1019 | −66 | 8 | 20 | TRTPISTYSDSYR |
| 2003.9565 | 2003.9475 | −.009 | −4 | 245 | 262 | NAVCCYNSPAVIL |
| | | | | | | PISEP |
| 2139.0686 | 2139.0762 | .0076 | 4 | 154 | 171 | LNAYEREVMVNML |
| | | | | | | NSLSR |
| 2441.1555 | 2441.1079 | −.0476 | −19 | 66 | 86 | CAVQDYTYRGSIS |
| | | | | | | GHPYL |
| | | | | | | PEK |

| | |
|---|---|
| MDN1, midasin homolog (yeast) [Homo sapiens] | gi\|55958030 |

Protein Group

| | |
|---|---|
| MDN1, midasin homolog (yeast) [Homo sapiens] | gi\|55958512 |

Peptide Information

| | | | | Start | End | |
|---|---|---|---|---|---|---|
| Calc. Mass | Obsrv. Mass | ±da | ±ppm | Seq. | Seq. | Sequence |
| 804.4322 | 804.384 | −.0482 | −60 | 23 | 29 | SRSELGR |
| 808.4563 | 808.3871 | −.0692 | −86 | 952 | 958 | QPIPEPK |
| 818.4366 | 818.3859 | −.0507 | −62 | 701 | 708 | SAVNKDGK |
| 824.4005 | 824.4156 | .0151 | 18 | 94 | 100 | LCVSMSK |
| 827.437 | 827.395 | −.042 | −51 | 178 | 184 | SHDTLVR |
| 828.4686 | 828.4219 | −.0467 | −56 | 464 | 470 | RPTLEGR |
| 834.4389 | 834.3736 | −.0653 | −78 | 520 | 527 | MAEVIGSK |
| 839.4522 | 839.3781 | −.0741 | −88 | 688 | 693 | WHDLLR |
| 848.5101 | 848.4327 | −.0774 | −91 | 1133 | 1139 | VLSRAFR |
| 922.5581 | 922.4904 | −.0677 | −73 | 819 | 826 | RNLPPGIR |
| 1003.524 | 1003.5698 | .0458 | 46 | 1062 | 1070 | EGVLIDAMR |
| 1003.524 | 1003.5698 | .0458 | 46 | 1062 | 1070 | EGVLIDAMR |
| 1183.6028 | 1183.649 | .0462 | 39 | 301 | 310 | SYVLVESVCK |
| 1546.7424 | 1546.6477 | −.0947 | −61 | 657 | 669 | EAFEELFAQTFSK |
| 1546.7424 | 1546.6477 | −.0947 | −61 | 657 | 669 | EAFEELFAQTFSK |
| 1784.8789 | 1784.8806 | .0017 | 1 | 1116 | 1131 | FMLFATQNPPGLYGGR |

| | |
|---|---|
| immunoglobulin heavy chain [Homo sapiens] | gi\|46253956 |

Peptide Information

| | | | | Start | End | |
|---|---|---|---|---|---|---|
| Calc. Mass | Obsrv. Mass | ±da | ±ppm | Seq. | Seq. | Sequence |
| 817.4414 | 817.4075 | −.0339 | −41 | 1 | 8 | VQSGAEVK |
| 827.4594 | 827.395 | −.0644 | −78 | 64 | 69 | RPDQRR |
| 1514.8075 | 1514.6637 | −.1438 | −95 | 40 | 54 | EHSGPGLPGQGLL |
| | | | | | | PR |
| 2441.2896 | 2441.1079 | −.1817 | −74 | 40 | 63 | EHSGPGLPGQGLL |
| | | | | | | PRTG |
| | | | | | | DGVVELR |

| | |
|---|---|
| bromodomain and PHD finger containing, 1, | gi\|119584376 |
| isoform CRA_a [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 814.4094 | 814.4108 | .0014 | 2 | 257 | 262 | DTIFYR |
| 986.5206 | 986.5446 | .024 | 24 | 453 | 459 | TWQWLPR |
| 986.5206 | 986.5446 | .024 | 24 | 453 | 459 | TWQWLPR |
| 1158.5637 | 1158.632 | .0683 | 59 | 48 | 58 | ENTETPAATPK |
| 1158.5637 | 1158.632 | .0683 | 59 | 48 | 58 | ENTETPAATPK |
| 1211.6532 | 1211.7495 | .0963 | 79 | 257 | 266 | DTIFYRAAVR |
| 1875.9131 | 1875.8578 | −.0553 | −29 | 2 | 17 | SYAQAQRMVEVD |
| | | | | | | LHGR |
| 2096.1362 | 2095.9319 | −.2043 | −97 | 411 | 429 | EGMFHHGVPIPVP |
| | | | | | | PLEVLK |
| 2455.2036 | 2455.0674 | −.1362 | −55 | 206 | 225 | ETVPDYLDHIKKP |
| | | | | | | MDFFT |
| | | | | | | MK |
| 2487.1936 | 2487.0879 | −.1057 | −42 | 206 | 225 | ETVPDYLDHIKKP |
| | | | | | | MDFFT |
| | | | | | | MK |
| 2512.3203 | 2512.2356 | −.0847 | −34 | 408 | 429 | MPREGMFHHGVPI |
| | | | | | | PVPP |
| | | | | | | LEVLK |
| 2512.3203 | 2512.2356 | −.0847 | −34 | 408 | 429 | MPREGMFHHGVPI |
| | | | | | | PVPP |
| | | | | | | LEVLK |

| | |
|---|---|
| sciellin isoform b variant [Homo sapiens] | gi\|62896999 |

Peptide Information

| | Obsrv. | | | Start | End | |
|---|---|---|---|---|---|---|
| Calc. Mass | Mass | ±da | ±ppm | Seq. | Seq. | Sequence |
| 817.489 | 817.4075 | −.0815 | −100 | 2 | 8 | SNVTLRK |
| 828.4938 | 828.4219 | −.0719 | −87 | 396 | 402 | VTPEVKR |
| 832.4457 | 832.3846 | −.0611 | −73 | 336 | 342 | VNARMNK |
| 848.4407 | 848.4327 | −.008 | −9 | 336 | 342 | VNAR |
| | | | | | | MNK |
| 863.4621 | 863.4113 | −.0508 | −59 | 409 | 415 | DLNNF |
| | | | | | | IK |
| 986.5629 | 986.5446 | −.0183 | −19 | 80 | 88 | DVPKATISR |
| 986.5629 | 986.5446 | −.0183 | −19 | 80 | 88 | DVPKATISR |
| 1030.5415 | 1030.5361 | −.0054 | −5 | 251 | 259 | GEELDNLIK |
| 1030.5415 | 1030.5361 | −.0054 | −5 | 251 | 259 | GEELDNLIK |
| 1251.6368 | 1251.5927 | −.0441 | −35 | 38 | 47 | TFLQDNSWIK |
| 1514.7883 | 1514.6637 | −.1246 | −82 | 287 | 299 | AKSLESLIYMSTR |
| 1857.8656 | 1857.8433 | −.0223 | −12 | 619 | 633 | DMCTYCRKPL |
| | | | | | | GVETK |
| 1875.8915 | 1875.8578 | −.0337 | −18 | 671 | 685 | QTIHCEPCYPKI |
| | | | | | | MAK |
| 2082.0098 | 2081.9248 | −.085 | −41 | 544 | 561 | DQNLENLIEVN |
| | | | | | | SHVSENK |
| 2153.1672 | 2153.1255 | −.0417 | −19 | 521 | 539 | NNQSQDLDNLI |
| | | | | | | KVKPSALR |

| | | | |
|---|---|---|---|
| | 68/C17 | | Instr./Gel |
| | | | Origin |
| | [1] Sample Project | | Instrument |
| | 20110503 | | Sample |
| | | | Name |
| Protein Name- | KH16 (SEQ ID NO. KH16) | Accession No. | |

| | |
|---|---|
| unnamed protein product [Homo sapiens] | gi\|194391084 |

Peptide Information

| | | | | Start | End | |
|---|---|---|---|---|---|---|
| Calc. Mass | Obsrv. Mass | ±da | ±ppm | Seq. | Seq. | Sequence |
| 815.4985 | 815.4535 | −.045 | −55 | 24 | 31 | AVDAALKK |
| 861.407 | 861.4133 | .0063 | 7 | 251 | 257 | ICVGCPR |
| 861.407 | 861.4133 | .0063 | 7 | 251 | 257 | ICVGCPR |
| 1003.5684 | 1003.5696 | .0012 | 1 | 176 | 184 | QVVAGLNFR |
| 1003.5684 | 1003.5696 | .0012 | 1 | 176 | 184 | QVVAGLNFR |
| 1030.5356 | 1030.5363 | .0007 | 1 | 305 | 312 | YFIDFVAR |
| 1030.5356 | 1030.5363 | .0007 | 1 | 305 | 312 | YFIDFVAR |
| 1158.6306 | 1158.6313 | .0007 | 1 | 304 | 312 | KYFIDFVAR |
| 1251.5892 | 1251.595 | .0058 | 5 | 53 | 63 | TVGSDTFYSFK |
| 1321.653 | 1321.647 | −.006 | −5 | 90 | 102 | AATGECTATVGKR |
| 1514.6581 | 1514.6606 | .0025 | 2 | 78 | 89 | TWQDCEYKDAAK |
| 1784.8741 | 1784.8759 | .0018 | 1 | 53 | 67 | TVGSDTFYSFKYEIK |
| 1874.8781 | 1874.8535 | −.0246 | −13 | 32 | 46 | YNSQNQSNNQFV |
| | | | | | | LYR |
| 1874.8781 | 1874.8535 | −.0246 | −13 | 32 | 46 | YNSQNQSNNQFV |
| | | | | | | LYR |
| 2139.0815 | 2139.0833 | .0018 | 1 | 258 | 276 | DIPTNSPELEETLT |
| | | | | | | HTITK |
| | 2139.0833 | | | | | |
| 2441.1038 | 2441.0786 | −.0252 | −10 | 208 | 228 | SLWNGDT |
| | | | | | | GECTDNAYIDI |
| | | | | | | QLR |
| 2512.2178 | 2512.1931 | −.0247 | −10 | 229 | 250 | IASFSQNC |
| | | | | | | DIYPGKDFVQ |
| | | | | | | PPTK |
| 2512.2178 | 2512.1931 | −.0247 | −10 | 229 | 250 | IASFSQNCDIYPGKDFVQ |
| | | | | | | PPTK |

| | |
|---|---|
| kininogen 1, isoform CRA_a [Homo sapiens] | gi\|119598585 |

Protein Group

| | |
|---|---|
| Kininogen 1 [Homo sapiens] | gi\|37748641 |
| kininogen 1 variant [Homo sapiens] | gi\|62898910 |
| kininogen 1, isoform CRA_a [Homo sapiens] | gi\|119598587 |
| kininogen [Homo sapiens] | gi\|386853 |
| kininogen [Homo sapiens] | gi\|28932976 |
| kininogen [Homo sapiens] | |
| kininogen-1 isoform 2 [Homo sapiens] | gi\|4504893 |
| prepro alpha-2-thiol proteinase inhibitor [Homo sapiens] | gi\|177890 |
| unnamed protein product [Homo sapiens] | gi\|213506121 |
| unnamed protein product [Homo sapiens] | gi\|213506103 |
| unnamed protein product [Homo sapiens] | gi\|207100681 |
| unnamed protein product [Homo sapiens] | gi\|207100663 |
| unnamed protein product [Homo sapiens] | gi\|189054825 |
| unnamed protein product [Homo sapiens] | gi\|158255114 |

Peptide Information

| | | | | Start | End | |
|---|---|---|---|---|---|---|
| Calc. Mass | Obsrv. Mass | ±da | ±ppm | Seq. | Seq. | Sequence |
| 815.4985 | 815.4535 | −.045 | −55 | 36 | 43 | AVDAALKK |
| 861.407 | 861.4133 | .0063 | 7 | 263 | 269 | ICVGCPR |
| 861.407 | 861.4133 | .0063 | 7 | 263 | 269 | ICVGCPR |
| 1003.5684 | 1003.5696 | .0012 | 1 | 188 | 196 | QVVAGLNFR |
| 1003.5684 | 1003.5696 | .0012 | 1 | 188 | 196 | QVVAGLNFR |
| 1030.5356 | 1030.5363 | .0007 | 1 | 317 | 324 | YFIDFVAR |
| 1030.5356 | 1030.5363 | .0007 | 1 | 317 | 324 | YFIDFVAR |
| 1158.6306 | 1158.6313 | .0007 | 1 | 316 | 324 | KYFIDFVAR |
| 1251.5892 | 1251.595 | .0058 | 5 | 65 | 75 | TVGSDTFYSFK |
| 1321.653 | 1321.647 | −.006 | −5 | 102 | 114 | AATGECTATVGKR |
| 1514.6581 | 1514.6606 | .0025 | 2 | 90 | 101 | TWQDCEYKDAAK |
| 1784.8741 | 1784.8759 | .0018 | 1 | 65 | 79 | TVGSDTFYSFKYEIK |
| 1874.8781 | 1874.8535 | −.0246 | −13 | 44 | 58 | YNSQNQSNNQFV |
| | | | | | | LYR |
| 1874.8781 | 1874.8535 | −.0246 | −13 | 44 | 58 | YNSQNQSNNQFV |
| | | | | | | LYR |
| 2139.0815 | 2139.0833 | .0018 | 1 | 270 | 288 | DIPTNSPELEETLT |
| | | | | | | HTITK |
| 2441.1038 | 2441.0786 | −.0252 | −10 | 220 | 240 | SLWNGDTGEC |
| | | | | | | TDNAYIDI |
| | | | | | | QLR |
| 2512.2178 | 2512.1931 | −.0247 | −10 | 241 | 262 | IASFSQNCDIYP |
| | | | | | | GKDFVQ |
| | | | | | | PPTK |
| 2512.2178 | 2512.1931 | −.0247 | −10 | 241 | 262 | IASFSQNCDIYP |
| | | | | | | GKDFVQ PPTK |

| | |
|---|---|
| RecName: Full = Kininogen-1; AltName: | gi\|124056474 |

Protein Group

| | |
|---|---|
| Full = Alpha-2-thiol proteinase | |
| inhibitor; AltName: Full = Fitzgerald | |
| factor; AltName: Full = High | |
| molecular | |
| weight kininogen; | |
| Short = HMWK; | |
| AltName: | |
| Full = Williams-Fitzgerald-Flaujeac factor; Contains: | |
| RecName: Full = Kininogen-1 heavy | |
| chain; Contains: RecName: | |
| Full = T-kinin; AltName: | |
| Full = Ile-Ser- | |
| Bradykinin; Contains: | |
| RecName: | |
| Full = Bradykinin; AltName: Full = Kallidin I; Contains: | |
| RecName: Full = Lysyl- | |
| bradykinin; AltName: | |
| Full = Kallidin | |
| II; Contains: RecName: Full = Kininogen-1 light chain; | |
| Contains: RecName: Full = Low | |
| molecular weight growth- | |
| promoting factor; Flags: | |
| Precursor | |
| kininogen 1, isoform CRA_b [Homo sapiens] | gi\|119598586 |
| kininogen [Homo sapiens] | gi\|386852 |
| kininogen-1 isoform 1 [Homo sapiens] | gi\|156231037 |
| unnamed protein product [Homo sapiens] | gi\|298886629 |
| unnamed protein product [Homo sapiens] | |
| unnamed protein product [Homo sapiens] | gi\|194383526 |

Peptide Information

| | | | | Start | End | |
|---|---|---|---|---|---|---|
| Calc. Mass | Obsrv. Mass | ±da | ±ppm | Seq. | Seq. | Sequence |
| 815.4985 | 815.4535 | −.045 | −55 | 36 | 43 | AVDAALKK |
| 861.407 | 861.4133 | .0063 | 7 | 263 | 269 | ICVGCPR |
| 861.407 | 861.4133 | .0063 | 7 | 263 | 269 | ICVGCPR |
| 1003.5684 | 1003.5696 | .0012 | 1 | 188 | 196 | QVVAGLNFR |
| 1003.5684 | 1003.5696 | .0012 | 1 | 188 | 196 | QVVAGLNFR |
| 1030.5356 | 1030.5363 | .0007 | 1 | 317 | 324 | YFIDFVAR |
| 1030.5356 | 1030.5363 | .0007 | 1 | 317 | 324 | YFIDFVAR |
| 1158.6306 | 1158.6313 | .0007 | 1 | 316 | 324 | KYFIDFVAR |
| 1251.5892 | 1251.595 | .0058 | 5 | 65 | 75 | TVGSDTFYSFK |
| 1321.653 | 1321.647 | −.006 | −5 | 102 | 114 | AATGECTATVGKR |
| 1514.6581 | 1514.6606 | .0025 | 2 | 90 | 101 | TWQDCEYKDAAK |
| 1784.8741 | 1784.8759 | .0018 | 1 | 65 | 79 | TVGSDTFYSFKYEIK |
| 1874.8781 | 1874.8535 | −.0246 | −13 | 44 | 58 | YNSQNQSNNQFV |
| | | | | | | LYR |
| 1874.8781 | 1874.8535 | −.0246 | −13 | 44 | 58 | YNSQNQSNNQFV |
| | | | | | | LYR |
| 2139.0815 | 2139.0833 | .0018 | 1 | 270 | 288 | DIPTNSPELEETLT |
| | | | | | | HTITK |
| 2441.1038 | 2441.0786 | −.0252 | −10 | 220 | 240 | SLWNGDTGECTD |
| | | | | | | NAYIDI |
| | | | | | | QLR |
| 2512.2178 | 2512.1931 | −.0247 | −10 | 241 | 262 | IASFSQNCDIYPGK |
| | | | | | | DFVQ |
| | | | | | | PPTK |
| 2512.2178 | 2512.1931 | −.0247 | −10 | 241 | 262 | IASFSQNCDIYPGK |
| | | | | | | DFVQ |
| | | | | | | PPTK |

| | |
|---|---|
| kininogen-1 isoform 3 [Homo sapiens] | gi\|262050546 |

Peptide Information

| | | | | Start | End | |
|---|---|---|---|---|---|---|
| Calc. Mass | Obsrv. Mass | ±da | ±ppm | Seq. | Seq. | Sequence |
| 815.4985 | 815.4535 | −.045 | −55 | 36 | 43 | AVDAALKK |
| 861.407 | 861.4133 | .0063 | 7 | 227 | 233 | ICVGCPR |
| 861.407 | 861.4133 | .0063 | 7 | 227 | 233 | ICVGCPR |
| 1030.5356 | 1030.5363 | .0007 | 1 | 281 | 288 | YFIDFVAR |
| 1030.5356 | 1030.5363 | .0007 | 1 | 281 | 288 | YFIDFVAR |
| 1158.6306 | 1158.6313 | .0007 | 1 | 280 | 288 | KYFIDFVAR |
| 1251.5892 | 1251.595 | .0058 | 5 | 65 | 75 | TVGSDTFYSFK |
| 1321.653 | 1321.647 | −.006 | −5 | 102 | 114 | AATGECTATVGKR |
| 1514.6581 | 1514.6606 | .0025 | 2 | 90 | 101 | TWQDCEYKDAAK |
| 1784.8741 | 1784.8759 | .0018 | 1 | 65 | 79 | TVGSDTFYSFKYEIK |
| 1874.8781 | 1874.8535 | −.0246 | −13 | 44 | 58 | YNSQNQSNNQFV |
| | | | | | | LYR |
| 1874.8781 | 1874.8535 | −.0246 | −13 | 44 | 58 | YNSQNQSNNQFV |
| | | | | | | LYR |
| 2139.0815 | 2139.0833 | .0018 | 1 | 234 | 252 | DIPTNSPELEETLT |
| | | | | | | HTITK |
| 2512.2178 | 2512.1931 | −.0247 | −10 | 205 | 226 | IASFSQNCDIYPGK |
| | | | | | | DFVQ |
| | | | | | | PPTK |
| 2512.2178 | 2512.1931 | −.0247 | −10 | 205 | 226 | IASFSQNCDIYPGK |
| | | | | | | DFVQ |
| | | | | | | PPTK |

| | |
|---|---|
| KNG1 protein [Homo sapiens] | gi\|116283732 |

Peptide Information

| | | | | Start | End | |
|---|---|---|---|---|---|---|
| Calc. Mass | Obsrv. Mass | ±da | ±ppm | Seq. | Seq. | Sequence |
| 861.407 | 861.4133 | .0063 | 7 | 127 | 133 | ICVGCPR |
| 861.407 | 861.4133 | .0063 | 7 | 127 | 133 | ICVGCPR |
| 1003.5684 | 1003.5696 | .0012 | 1 | 52 | 60 | QVVAGLNFR |
| 1003.5684 | 1003.5696 | .0012 | 1 | 52 | 60 | QVVAGLNFR |
| 1030.5356 | 1030.5363 | .0007 | 1 | 181 | 188 | YFIDFVAR |
| 1030.5356 | 1030.5363 | .0007 | 1 | 181 | 188 | YFIDFVAR |
| 1158.6306 | 1158.6313 | .0007 | 1 | 180 | 188 | KYFIDFVAR |
| 2139.0815 | 2139.0833 | .0018 | 1 | 134 | 152 | DIPTNSPELEETLT |
| | | | | | | HTITK |
| 2441.1038 | 2441.0786 | −.0252 | −10 | 84 | 104 | SLWNGDTGECTD |
| | | | | | | NAYIDI |
| | | | | | | QLR |
| 2512.2178 | 2512.1931 | −.0247 | −10 | 105 | 126 | IASFSQNCDIYPGK |
| | | | | | | DFVQ |
| | | | | | | PPTK |
| 2512.2178 | 2512.1931 | −.0247 | −10 | 105 | 126 | IASFSQNCDIYPGK |
| | | | | | | DFVQ |
| | | | | | | PPTK |

| | |
|---|---|
| unnamed protein product [Homo sapiens] | gi\|193784739 |

Peptide Information

| | Obsrv. | | | Start | End | |
|---|---|---|---|---|---|---|
| Calc. Mass | Mass | ±da | ±ppm | Seq. | Seq. | Sequence |
| 823.4607 | 823.4086 | −.0521 | −63 | 2 | 8 | PLGHIMR |
| 836.4413 | 836.3985 | −.0428 | −51 | 225 | 230 | FWNLTR |
| 857.4515 | 857.4417 | −.0098 | −11 | 112 | 117 | YYVKER |
| 865.3785 | 865.4606 | .0821 | 95 | 232 | 239 | TESALTED |
| 870.5083 | 870.5379 | .0296 | 34 | 109 | 115 | LGKYYVK |
| 986.491 | 986.5456 | .0546 | 55 | 1 | 8 | MPLGHIMR |
| 986.491 | 986.5456 | .0546 | 55 | 1 | 8 | MPLGHIMR |
| 2095.9429 | 2095.9111 | −.0318 | −15 | 206 | 222 | YAMTVWYFDA |
| | | | | | | EERAEAK |
| 2473.2551 | 2473.0457 | −.2094 | −85 | 173 | 192 | SFIADVEPIFDR |
| | | | | | | LLFFWSDR |
| 2473.2551 | 2473.0457 | −.2094 | −85 | 173 | 192 | SFIADVEPIFDR |
| | | | | | | LLFFWSDR |

| | |
|---|---|
| sciellin isoform b variant [Homo sapiens] | gi\|162896999 |

Peptide Information

| | Obsrv. | | | Start | End | |
|---|---|---|---|---|---|---|
| Calc. Mass | Mass | ±da | ±ppm | Seq. | Seq. | Sequence |
| 817.489 | 817.4197 | −.0693 | −85 | 2 | 8 | SNVTLRK |
| 820.4345 | 820.3947 | −.0398 | −49 | 1 | 7 | MSNVTLR |
| 836.4294 | 836.3985 | −.0309 | −37 | 1 | 7 | MSNVTLR |
| 840.4097 | 840.4077 | −.002 | −2 | 583 | 589 | TYVENSK |
| 848.4407 | 848.4392 | −.0015 | −2 | 336 | 342 | VNARMNK |
| 860.4584 | 860.4324 | −.026 | −30 | 316 | 322 | VNQRTDK |
| 863.4621 | 863.4159 | −.0462 | −54 | 409 | 415 | DLNNFIK |
| 986.5629 | 986.5456 | −.0173 | −18 | 80 | 88 | DVPKATISR |
| 986.5629 | 986.5456 | −.0173 | −18 | 80 | 88 | DVPKATISR |
| 1030.5415 | 1030.5363 | −.0052 | −5 | 251 | 259 | GEELDNLIK |
| 1030.5415 | 1030.5363 | −.0052 | −5 | 251 | 259 | GEELDNLIK |
| 1251.6368 | 1251.595 | −.0418 | −33 | 38 | 47 | TFLQDNSWIK |
| 1514.7883 | 1514.6606 | −.1277 | −84 | 287 | 299 | AKSLESLIYMSTR |
| 1857.8656 | 1857.8317 | −.0339 | −18 | 619 | 633 | DMCTYCRKPLGV |
| | | | | | | ETK |
| 1875.8915 | 1875.8496 | −.0419 | −22 | 671 | 685 | QTIHCEPCYPKIMAK |
| 2082.0098 | 2081.9111 | −.0987 | −47 | 544 | 561 | DQNLENLIEVNSH |
| | | | | | | VSENK |
| 2153.1672 | 2153.0808 | −.0864 | −40 | 521 | 539 | NNQSQDLDNLIKV |
| | | | | | | KPSALR |

| | |
|---|---|
| unnamed protein product [Homo sapiens] | gi\|40031588 |

Protein Group

| | |
|---|---|
| chromosome 17 open reading frame 25, isoform | gi\|119611054 |
| CRA_e [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 804.3846 | 804.4047 | .0201 | 25 | 167 | 172 | DEEKQR |
| 840.4362 | 840.4077 | −.0285 | −34 | 125 | 130 | FYLQNR |
| 861.4981 | 861.4133 | −.0848 | −98 | 6 | 12 | ALHFVFK |
| 861.4981 | 861.4133 | −.0848 | −98 | 6 | 12 | ALHFVFK |
| 1030.6078 | 1030.5363 | −.0715 | −69 | 25 | 33 | DVLGMKVLR |
| 1030.6078 | 1030.5363 | −.0715 | −69 | 25 | 33 | DVLGMKVLR |
| 1053.5099 | 1053.5475 | .0376 | 36 | 163 | 170 | IYEKDEEK |
| 1514.713 | 1514.6606 | −.0524 | −35 | 151 | 162 | SLNYWCNLLGMK |
| 2068.0127 | 2067.8892 | −.1235 | −60 | 82 | 101 | LGNDFMGITLASS |
| | | | | | | QAVSN |
| | | | | | | AR |

| | |
|---|---|
| unnamed protein product [Homo sapiens] | gi\|13515362 |

Peptide Information

| | Obsrv. | | | Start | End | |
|---|---|---|---|---|---|---|
| Calc. Mass | Mass | ±da | ±ppm | Seq. | Seq. | Sequence |
| 808.4199 | 808.4188 | −.0011 | −1 | 674 | 679 | EQYLQK |
| 814.4094 | 814.4131 | .0037 | 5 | 338 | 343 | IYFDTR |
| 817.405 | 817.4197 | .0147 | 18 | 1537 | 1543 | DDEVAIR |
| 818.4267 | 818.4182 | −.0085 | −10 | 370 | 375 | WQNSKR |
| 820.344 | 820.3947 | .0507 | 62 | 1389 | 1394 | CQQPCK |
| 825.4287 | 825.44 | .0113 | 14 | 476 | 482 | YLLMGGR |
| 830.4267 | 830.414 | −.0127 | −15 | 997 | 1002 | LNYQHR |
| 844.504 | 844.4724 | −.0316 | −37 | 990 | 996 | VNIPFVR |
| 863.3424 | 863.4159 | .0735 | 85 | 35 | 41 | NGNQDCR |
| 864.4547 | 864.4229 | −.0318 | −37 | 4 | 10 | NPHQGRR |
| 870.4866 | 870.5379 | .0513 | 59 | 1396 | 1402 | LLICSHK |
| 986.5768 | 986.5456 | −.0312 | −32 | 1568 | 1575 | LEEIEIIK |
| 986.5768 | 986.5456 | −.0312 | −32 | 1568 | 1575 | LEEIEIIK |
| 1251.5419 | 1251.595 | .0531 | 42 | 642 | 651 | AYMSIMTQMK |
| 1858.0123 | 1857.8317 | −.1806 | −97 | 1761 | 1776 | VQIVSAIGYPR |
| | | | | | | GHWFK |
| 2082.0059 | 2081.9111 | −.0948 | −46 | 1729 | 1745 | FTQEDEQLVQE |
| | | | | | | KMEALK |
| 2153.1292 | 2153.0808 | −.0484 | −22 | 859 | 874 | WQLYRLWLQL |
| | | | | | | YQADTR |
| 2441.1145 | 2441.0786 | −.0359 | −15 | 642 | 662 | AYMSIMTQMKE |
| | | | | | | SEQELH |
| | | | | | | EGAK |
| 2455.2861 | 2455.0752 | −.2109 | −86 | 896 | 918 | LQEDLHILKDA |
| | | | | | | QVVGMTT |
| | | | | | | TGAAK |
| 2473.1045 | 2473.0457 | −.0588 | −24 | 642 | 662 | AYMSIMTQMKE |
| | | | | | | SEQELH |
| | | | | | | EGAK |
| 2473.1045 | 2473.0457 | −.0588 | −24 | 642 | 662 | AYMSIMTQMKE |
| | | | | | | SEQELH |
| | | | | | | EGAK |

| | |
|---|---|
| MSTP102 [Homo sapiens] | gi\|33338010 |

Peptide Information

| | Obsrv. | | | Start | End | |
|---|---|---|---|---|---|---|
| Calc. Mass | Mass | ±da | ±ppm | Seq. | Seq. | Sequence |
| 860.4737 | 860.4324 | −.0413 | −48 | 8 | 14 | RQNAFPK |
| 1053.5583 | 1053.5475 | −.0108 | −10 | 1 | 8 | MPYLMVKR |
| 2455.2036 | 2455.0752 | −.1284 | −52 | 17 | 38 | MSVFTISVQHYAD |
| | | | | | | GSSQL |
| | | | | | | GQKR |
| 2512.25 | 2512.1931 | −.0569 | −23 | 15 | 37 | TKMSVFTISVQHY |
| | | | | | | ADGSS |
| | | | | | | QLGQK |
| 2512.25 | 2512.1931 | −.0569 | −23 | 15 | 37 | TKMSVFTISVQHY |
| | | | | | | ADGSS |
| | | | | | | QLGQK |

| | |
|---|---|
| immunoglobulin heavy chain variable region | gi\|66821381 |
| [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 817.4566 | 817.4197 | −.0369 | −45 | 106 | 111 | WELLTR |
| 820.373 | 820.3947 | .0217 | 26 | 97 | 102 | ECARER |
| 830.4268 | 830.414 | −.0128 | −15 | 1 | 9 | GAAGGVWGR |
| 836.4737 | 836.3985 | −.0752 | −90 | 64 | 70 | GRFTISR |
| 986.5278 | 986.5456 | .0178 | 18 | 1 | 10 | GAAGGVWGRR |
| 986.5278 | 986.5456 | .0178 | 18 | 1 | 10 | GAAGGVWGRR |
| 2096.0271 | 2095.9111 | −.116 | −55 | 106 | 123 | WELLTRGAFDIWG |
| | | | | | | QGTMV |

| | |
|---|---|
| OSBPL8 protein [Homo sapiens] | gi\|51895890 |

Peptide Information

| | Obsrv. | | | Start | End | |
|---|---|---|---|---|---|---|
| Calc. Mass | Mass | ±da | ±ppm | Seq. | Seq. | Sequence |
| 820.441 | 820.3947 | −.0463 | −56 | 13 | 20 | TSLLGDSK |
| 822.3588 | 822.3813 | .0225 | 27 | 247 | 254 | ATSESDGR |
| 834.425 | 834.4084 | −.0166 | −20 | 43 | 49 | MSQRQGK |
| 850.4199 | 850.4161 | −.0038 | −4 | 43 | 49 | MSQRQGK |
| 863.4767 | 863.4159 | −.0608 | −70 | 266 | 272 | CSSLLKR |
| 1053.5422 | 1053.5475 | .0053 | 5 | 108 | 117 | DSSTSSKLTK |
| 1546.7708 | 1546.6415 | −.1293 | −84 | 693 | 706 | AINAKDQTEATQEK |
| 1546.7708 | 1546.6415 | −.1293 | −84 | 693 | 706 | AINAKDQTEATQEK |
| 1875.7297 | 1875.8496 | .1199 | 64 | 334 | 349 | ENDQEHDESDNE |
| | | | | | | VMGK |
| 1890.9232 | 1890.8475 | −.0757 | −40 | 649 | 664 | KTDNSEVFWNPTP |
| | | | | | | DIK |
| 2153.0002 | 2153.0808 | .0806 | 37 | 247 | 265 | ATSESDGRCWMD |
| | | | | | | ALELA |
| | | | | | | LK |
| 2487.2766 | 2487.0583 | −.2183 | −88 | 627 | 648 | LGKEVLATLEGHW |
| | | | | | | DSEV |
| | | | | | | FITDK |
| 2487.2766 | 2487.0583 | −.2183 | −88 | 627 | 648 | LGKEVLATLEGHW |
| | | | | | | DSEV |
| | | | | | | FITDK |
| 2512.334 | 2512.1931 | −.1409 | −56 | 184 | 205 | NGQWVGTVLLNA |
| | | | | | | CEIIER |
| | | | | | | PSKK |
| 2512.334 | 2512.1931 | −.1409 | −56 | 184 | 205 | NGQWVGTVLLNACEIIER |
| | | | | | | PSKK |

| | |
|---|---|
| unnamed protein product [Homo sapiens] | gi\|11191031 |

Peptide Information

| | Obsrv. | | | Start | End | |
|---|---|---|---|---|---|---|
| Calc. Mass | Mass | ±da | ±ppm | Seq. | Seq. | Sequence |
| 823.4607 | 823.4086 | −.0521 | −63 | 2 | 8 | PLGHIMR |
| 857.4515 | 857.4417 | −.0098 | −11 | 112 | 117 | YYVKER |
| 865.3785 | 865.4606 | .0821 | 95 | 232 | 239 | TESALTED |
| 870.5083 | 870.5379 | .0296 | 34 | 109 | 115 | LGKYYVK |
| 986.491 | 986.5456 | .0546 | 55 | 1 | 8 | MPLGHIMR |
| 986.491 | 986.5456 | .0546 | 55 | 1 | 8 | MPLGHIMR |
| 2095.9429 | 2095.9111 | −.0318 | −15 | 206 | 222 | YAMTVWYFDAEE |
| | | | | | | RAEAK |
| 2473.2551 | 2473.0457 | −.2094 | −85 | 173 | 192 | SFIADVEPIFDRLL |
| | | | | | | FFWSDR |
| 2473.2551 | 2473.0457 | −.2094 | −85 | 173 | 192 | SFIADVEPIFDRLL |
| | | | | | | FFWSDR |

| | |
|---|---|
| unnamed protein product [Homo sapiens] | gi\|10439719 |

Peptide Information

| | Obsrv. | | | Start | End | |
|---|---|---|---|---|---|---|
| Calc. Mass | Mass | ±da | ±ppm | Seq. | Seq. | Sequence |
| 806.444 | 806.3729 | −.0711 | −88 | 160 | 166 | QSMVTLK |
| 818.473 | 818.4182 | −.0548 | −67 | 421 | 427 | ETSVVRK |
| 822.439 | 822.3813 | −.0577 | −70 | 160 | 166 | QSMVTLK |
| | 822.3813 | | | | | |
| 844.5251 | 844.4724 | −.0527 | −62 | 380 | 388 | LGSGGVVKK |
| 850.3975 | 850.4161 | .0186 | 22 | 79 | 85 | EELNMSK |
| 860.4836 | 860.4324 | −.0512 | −60 | 119 | 126 | TVSPSTIR |
| 1874.9753 | 1874.8535 | −.1218 | −65 | 89 | 104 | TIQQNITEQASVM |
| | | | | | | QKR |
| 1874.9753 | 1874.8535 | −.1218 | −65 | 89 | 104 | TIQQNITEQASVM |
| | | | | | | QKR |
| 1890.9702 | 1890.8475 | −.1227 | −65 | 89 | 104 | TIQQNITEQASVM |
| | | | | | | QKR |
| 2068.0508 | 2067.8892 | −.1616 | −78 | 287 | 304 | GLPHMFQQGGVF |
| | | | | | | YSIMKK |
| 2455.304 | 2455.0752 | −.2288 | −93 | 50 | 72 | VGDVQGQESESQ |
| | | | | | | LPTKII |
| | | | | | | LTGQK |
| 2512.1445 | 2512.1931 | .0486 | 19 | 266 | 286 | EMYSLCLLATLMG |
| | | | | | | EDVW |
| | | | | | | SYAK |
| 2512.1445 | 2512.1931 | .0486 | 19 | 266 | 286 | EMYSLCLLATLMG |
| | | | | | | EDVW |
| | | | | | | SYAK |

| | |
|---|---|
| egl nine homolog 3 [Homo sapiens] | gi\|11545787 |

Protein Group

| | |
|---|---|
| EGLN3 protein [Homo sapiens] | gi\|130267189 |
| EGLN3 protein [Homo sapiens] | gi\|14547150 |
| Egl nine homolog 3 (C. elegans) [Homo sapiens] | gi\|127798639 |
| Egl nine homolog 3 (C. elegans) [Homo sapiens] | gi\|127796436 |
| Egl nine homolog 3 (C. elegans) [Homo sapiens] | |
| Egl nine homolog 3 (C. elegans) [Homo sapiens] | gi\|77748083 |
| RecName: Full = Egl nine homolog 3; AltName: | gi\|32129515 |
| Full = HPH-1; AltName: | |
| Full = Hypoxia-inducible factor prolyl | |
| hydroxylase 3; Short = HIF-PH3; | |
| Short = HIF- | |
| prolyl | |
| hydroxylase 3; Short = HPH-3; AltName: Full = Prolyl | |
| hydroxylase domain- | |
| containing protein 3; | |
| Short = PHD3 | |
| egl nine homolog 3 (C. elegans) [Homo sapiens] | gi\|119586333 |
| unnamed protein product [Homo sapiens] | gi\|10437756 |
| unnamed protein product [Homo sapiens] | gi\|40216521 |
| unnamed protein product [Homo sapiens] | gi\|25897867 |

Peptide Information

| | | | | Start | End | |
|---|---|---|---|---|---|---|
| Calc. Mass | Obsrv. Mass | ±da | ±ppm | Seq. | Seq. | Sequence |
| 823.4607 | 823.4086 | −.0521 | −63 | 2 | 8 | PLGHIMR |
| 857.4515 | 857.4417 | −.0098 | −11 | 112 | 117 | YYVKER |
| 865.3785 | 865.4606 | .0821 | 95 | 232 | 239 | TESALTED |
| 870.5083 | 870.5379 | .0296 | 34 | 109 | 115 | LGKYYVK |
| 986.491 | 986.5456 | .0546 | 55 | 1 | 8 | MPLGHIMR |
| 986.491 | 986.5456 | .0546 | 55 | 1 | 8 | MPLGHIMR |
| 2095.9429 | 2095.9111 | −.0318 | −15 | 206 | 222 | YAMTVWYFDA |
| | | | | | | EERAEAK |
| 2473.2551 | 2473.0457 | −.2094 | −85 | 173 | 192 | SFIADVEPIFDR |
| | | | | | | LLFFWSDR |
| 2473.2551 | 2473.0457 | −.2094 | −85 | 173 | 192 | SFIADVEPIFDR |
| | | | | | | LLFFWSDR |

| | |
|---|---|
| unnamed protein product [Homo sapiens] | gi\|21439430 |

Peptide Information

| | Obsrv. | | | Start | End | |
|---|---|---|---|---|---|---|
| Calc. Mass | Mass | ±da | ±ppm | Seq. | Seq. | Sequence |
| 800.4413 | 800.4009 | −.0404 | −50 | 17 | 23 | LSPWAAR |
| 806.4003 | 806.3729 | −.0274 | −34 | 192 | 198 | SDAETKR |
| 814.4278 | 814.4131 | −.0147 | −18 | 24 | 30 | STHRGTR |
| 836.3897 | 836.3985 | .0088 | 11 | 178 | 184 | LGDDFNR |
| 848.4876 | 848.4392 | −.0484 | −57 | 213 | 219 | QEWFVK |
| 872.506 | 872.4337 | −.0723 | −83 | 95 | 102 | LLRNSGGR |
| 1251.5237 | 1251.595 | .0713 | 57 | 2 | 12 | AESSSESDHFR |
| 1321.6495 | 1321.647 | −.0025 | −2 | 178 | 188 | LGDDFNRELSR |

| | |
|---|---|
| hypothetical protein [Homo sapiens] | gi\|51476912 |

Peptide Information

| | Obsrv. | | | Start | End | |
|---|---|---|---|---|---|---|
| Calc. Mass | Mass | ±da | ±ppm | Seq. | Seq. | Sequence |
| 817.4964 | 817.4197 | −.0767 | −94 | 283 | 289 | GCIKVLK |
| | 817.4197 | | | | | |
| 822.4389 | 822.3813 | −.0576 | −70 | 276 | 282 | TSKIDMK |
| 836.436 | 836.3985 | −.0375 | −45 | 200 | 207 | TLSDATTK |
| 863.4655 | 863.4159 | −.0496 | −57 | 318 | 324 | QIKSMLQ |
| 1857.9202 | 1857.8317 | −.0885 | −48 | 58 | 74 | GAGHEIREAIQ |
| | | | | | | HPADEK |
| 1875.8806 | 1875.8496 | −.031 | −17 | 147 | 161 | MTNPAIQNDFS |
| | | | | | | YYRR |
| 2096.0002 | 2095.9111 | −.0891 | −43 | 168 | 186 | INNVPAEGENE |
| | | | | | | VNNELANR |
| 2153.1577 | 2153.0808 | −.0769 | −36 | 256 | 275 | VMVGVIILYDHV |
| | | | | | | HPAGAF |
| | | | | | | AK |

| | |
|---|---|
| tripartite motif-containing 14, isoform CRA_b | gi\|119579275 |
| [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 830.4301 | 830.414 | −.0161 | −19 | 1 | 7 | MPPRSSR |
| 834.3951 | 834.4084 | .0133 | 16 | 8 | 15 | QANAESSK |
| 850.4781 | 850.4161 | −.062 | −73 | 20 | 26 | GKFTELR |
| 1211.6855 | 1211.7461 | .0606 | 50 | 150 | 160 | VSSPRLVSNRP |
| 1890.0219 | 1889.8499 | −.172 | −91 | 22 | 37 | FTELRLLLDEEEAL |
| | | | | | | AK |
| 2082.0034 | 2081.9111 | −.0923 | −44 | 43 | 59 | NTQLTLQVYREQA |
| | | | | | | DSCR |

| | |
|---|---|
| unnamed protein product | gi\|40035634 |
| [Homo sapiens] | |

Protein Group

| | |
|---|---|
| unnamed protein product [Homo sapiens] | gi\|10435893 |

Peptide Information

| | | | | Start | End | |
|---|---|---|---|---|---|---|
| Calc. Mass | Obsrv. Mass | ±da | ±ppm | Seq. | Seq. | Sequence |
| 808.4199 | 808.4188 | −.0011 | −1 | 38 | 43 | EQYLQK |
| 817.405 | 817.4197 | .0147 | 18 | 901 | 907 | DDEVAIR |
| 818.4553 | 818.4182 | −.0371 | −45 | 509 | 515 | ICVALSR |
| 820.344 | 820.3947 | .0507 | 62 | 753 | 758 | CQQPCK |
| 830.4267 | 830.414 | −.0127 | −15 | 361 | 366 | LNYQHR |
| 844.504 | 844.4724 | −.0316 | −37 | 354 | 360 | VNIPFVR |
| 870.4866 | 870.5379 | .0513 | 59 | 760 | 766 | LLICSHK |
| 986.5768 | 986.5456 | −.0312 | −32 | 932 | 939 | LEEIEIIK |
| 986.5768 | 986.5456 | −.0312 | −32 | 932 | 939 | LEEIEIIK |
| 1251.5419 | 1251.595 | .0531 | 42 | 6 | 15 | AYMSIMTQMK |
| 2082.0059 | 2081.9111 | −.0948 | −46 | 1093 | 1109 | FTQEDEQLVQE |
| | | | | | | KMEALK |
| 2153.1292 | 2153.0808 | −.0484 | −22 | 223 | 238 | WQLYRLWLQL |
| | | | | | | YQADTR |
| 2441.1145 | 2441.0786 | −.0359 | −15 | 6 | 26 | AYMSIMTQMKE |
| | | | | | | SEQELH |
| | | | | | | EGAK |
| 2455.2861 | 2455.0752 | −.2109 | −86 | 260 | 282 | LQEDLHILKDA |
| | | | | | | QVVGMTT |
| | | | | | | TGAAK |
| 2473.1045 | 2473.0457 | −.0588 | −24 | 6 | 26 | AYMSIMTQMKE |
| | | | | | | SEQELH |
| | | | | | | EGAK |
| 2473.1045 | 2473.0457 | −.0588 | −24 | 6 | 26 | AYMSIMTQMKE |
| | | | | | | SEQELH |
| | | | | | | EGAK |

| | |
|---|---|
| myosin IB, isoform CRA_b [Homo sapiens] | gi\|119631245 |

Peptide Information

| Calc. | Obsrv. | | | Start | End | |
|---|---|---|---|---|---|---|
| Mass | Mass | ±da | ±ppm | Seq. | Seq. | Sequence |
| 816.4362 | 816.4129 | −.0233 | −29 | 512 | 518 | IQHYAGK |
| 820.4597 | 820.3947 | −.065 | −79 | 576 | 583 | ASVATLMK |
| 836.4368 | 836.3985 | −.0383 | −46 | 1 | 7 | MAKMEVK |
| 844.4523 | 844.4724 | .0201 | 24 | 132 | 139 | GAEVNQVK |
| 861.4312 | 861.4133 | −.0179 | −21 | 292 | 299 | VNGLDESK |
| 861.4424 | 861.4133 | −.0291 | −34 | 94 | 100 | SLRDQDK |
| 1514.7614 | 1514.6606 | −.1008 | −67 | 595 | 605 | YFWHMKLSQFK |
| 1857.9706 | 1857.8317 | −.1389 | −75 | 140 | 156 | EQLLQSNPVLEAF |
| | | | | | | GNAK |
| 1890.0266 | 1889.8499 | −.1767 | −93 | 566 | 583 | RPPTAGSQFKASV |
| | | | | | | ATLMK |
| 2139.0938 | 2139.0833 | −.0105 | −5 | 120 | 139 | LVMSYVAAVCGK |
| | | | | | | GAEVN |
| | | | | | | QVK |

| | | | |
|---|---|---|---|
| | 75/C24 | | Instr./Gel |
| | | | Origin |
| | [1] Sample Project | | Instrument |
| | 20110503 | | Sample |
| | | | Name |
| Protein Name- | KH17 (SEQ ID NO. KH17) | Accession No. | |

| | |
|---|---|
| unnamed protein product [Homo sapiens] | gi\|194376310 |

Peptide Information

| | Obsrv. | | | Start | End | |
|---|---|---|---|---|---|---|
| Calc. Mass | Mass | ±da | ±ppm | Seq. | Seq. | Sequence |
| 923.5672 | 923.5624 | −.0048 | −5 | 301 | 308 | IIAPPERK |
| 976.4483 | 976.4526 | .0043 | 4 | 19 | 28 | AGFAGDDAPR |
| 976.4483 | 976.4526 | .0043 | 4 | 19 | 28 | AGFAGDDAPR |
| 1036.6514 | 1036.6522 | .0008 | 1 | 299 | 307 | IKIIAPPER |
| 1132.527 | 1132.53 | .003 | 3 | 169 | 178 | GYSFTTTAER |
| 1132.527 | 1132.53 | .003 | 3 | 169 | 178 | GYSFTTTAER |
| 1198.7056 | 1198.7052 | −.0004 | 0 | 29 | 39 | AVFPSIVGRPR |
| 1198.7056 | 1198.7052 | −.0004 | 0 | 29 | 39 | AVFPSIVGRPR |
| | 1198.7052 | | | | | |
| 1516.7028 | 1516.7002 | −.0026 | −2 | 332 | 344 | QEYDESGPSIVHR |
| 1516.7028 | 1516.7002 | −.0026 | −2 | 332 | 344 | QEYDESGPSIVHR |
| 1629.8231 | 1629.8131 | −.01 | −6 | 169 | 182 | GYSFTTTAEREIVR |
| 1639.8361 | 1639.8319 | −.0042 | −3 | 150 | 163 | LDLAGRDLTDYLMK |
| 1790.892 | 1790.8784 | −.0136 | −8 | 211 | 226 | SYELPDGQVITIGNER |
| 1790.892 | 1790.8784 | −.0136 | −8 | 211 | 226 | SYELPDGQVITIGNER |
| 1954.0645 | 1954.0728 | .0083 | 4 | 68 | 85 | VAPEEHPVLLTEAPLNPK |
| 2231.0649 | 2231.0669 | .002 | 1 | 264 | 284 | DLYANTVLSGGTTMYPGI |
| | | | | | | ADR |

| | |
|---|---|
| unnamed protein product [Homo sapiens] | gi\|194388064 |

Peptide Information

| | Obsrv. | | | Start | End | |
|---|---|---|---|---|---|---|
| Calc. Mass | Mass | ±da | ±ppm | Seq. | Seq. | Sequence |
| 923.5672 | 923.5624 | −.0048 | −5 | 310 | 317 | IIAPPERK |
| 976.4483 | 976.4526 | .0043 | 4 | 19 | 28 | AGFAGDDAPR |
| 976.4483 | 976.4526 | .0043 | 4 | 19 | 28 | AGFAGDDAPR |
| 1036.6514 | 1036.6522 | .0008 | 1 | 308 | 316 | IKIIAPPER |
| 1132.527 | 1132.53 | .003 | 3 | 178 | 187 | GYSFTTTAER |
| 1132.527 | 1132.53 | .003 | 3 | 178 | 187 | GYSFTTTAER |
| 1198.7056 | 1198.7052 | −.0004 | 0 | 29 | 39 | AVFPSIVGRPR |
| 1198.7056 | 1198.7052 | −.0004 | 0 | 29 | 39 | AVFPSIVGRPR |
| 1516.7028 | 1516.7002 | −.0026 | −2 | 341 | 353 | QEYDESGPSIVHR |
| 1516.7028 | 1516.7002 | −.0026 | −2 | 341 | 353 | QEYDESGPSIVHR |
| 1629.8231 | 1629.8131 | −.01 | −6 | 178 | 191 | GYSFTTTAEREIVR |
| 1639.8361 | 1639.8319 | −.0042 | −3 | 159 | 172 | LDLAGRDLTDYLMK |
| 1790.892 | 1790.8784 | −.0136 | −8 | 220 | 235 | SYELPDGQVITIGN |
| | | | | | | ER |
| 1790.892 | 1790.8784 | −.0136 | −8 | 220 | 235 | SYELPDGQVITI |
| | | | | | | GNER |
| 1954.0645 | 1954.0728 | .0083 | 4 | 96 | 113 | VAPEEHPVLLT |
| | | | | | | EAPLNPK |
| 2231.0649 | 2231.0669 | .002 | 1 | 273 | 293 | DLYANTVLSGG |
| | | | | | | TTMYPGI ADR |

| | |
|---|---|
| ACTB protein [Homo sapiens] | gi\|15277503 |

Peptide Information

| | Obsrv. | | | Start | End | |
|---|---|---|---|---|---|---|
| Calc. Mass | Mass | ±da | ±ppm | Seq. | Seq. | Sequence |
| 923.5672 | 923.5624 | −.0048 | −5 | 314 | 321 | IIAPPERK |
| 976.4483 | 976.4526 | .0043 | 4 | 4 | 13 | AGFAGDDAPR |
| 976.4483 | 976.4526 | .0043 | 4 | 4 | 13 | AGFAGDDAPR |
| 1036.6514 | 1036.6522 | .0008 | 1 | 312 | 320 | IKIIAPPER |
| 1132.527 | 1132.53 | .003 | 3 | 182 | 191 | GYSFTTTAER |
| 1132.527 | 1132.53 | .003 | 3 | 182 | 191 | GYSFTTTAER |
| 1198.7056 | 1198.7052 | −.0004 | 0 | 14 | 24 | AVFPSIVGRPR |
| 1198.7056 | 1198.7052 | −.0004 | 0 | 14 | 24 | AVFPSIVGRPR |
| 1516.7028 | 1516.7002 | −.0026 | −2 | 345 | 357 | QEYDESGPSIV |
| | | | | | | HR |
| 1516.7028 | 1516.7002 | −.0026 | −2 | 345 | 357 | QEYDESGPSIV |
| | | | | | | HR |
| 1629.8231 | 1629.8131 | −.01 | −6 | 182 | 195 | GYSFTTTAERE |
| | | | | | | IVR |
| 1639.8361 | 1639.8319 | −.0042 | −3 | 163 | 176 | LDLAGRDLTDY |
| | | | | | | LMK |
| 1790.892 | 1790.8784 | −.0136 | −8 | 224 | 239 | SYELPDGQVITI |
| | | | | | | GNER |
| 1790.892 | 1790.8784 | −.0136 | −8 | 224 | 239 | SYELPDGQVITI |
| | | | | | | GNER |
| 1954.0645 | 1954.0728 | .0083 | 4 | 81 | 98 | VAPEEHPVLLT |
| | | | | | | EAPLNPK |
| 2231.0649 | 2231.0669 | .002 | 1 | 277 | 297 | DLYANTVLSGG |
| | | | | | | TTMYPGI |
| | | | | | | ADR |

| | |
|---|---|
| actin, beta [Homo sapiens] | gi\|14250401 |

Protein Group

| | |
|---|---|
| Chain A, Model Of Actin-Fimbrin Abd2 Complex | gi\|168177284 |
| Chain A, Model Of Alpha-Actinin Ch1 Bound To | gi\|295321909 |
| F-Actin | |
| Chain B, Model Of Alpha-Actinin Ch1 Bound To | gi\|295321910 |
| F-Actin | |
| Chain C, Model Of Alpha-Actinin Ch1 Bound To | gi\|295321911 |
| F-Actin | |
| Chain D, Model Of Alpha-Actinin Ch1 Bound To | gi\|295321912 |
| F-Actin | |
| Chain E, Model Of Alpha-Actinin Ch1 Bound To | gi\|295321913 |
| F-Actin | |
| Chain F, Model Of Alpha-Actinin Ch1 Bound To | gi\|295321914 |
| F-Actin | |
| Chain G, Model Of Alpha-Actinin Ch1 Bound To | gi\|295321915 |
| F-Actin | |
| Chain H, Model Of Alpha-Actinin Ch1 Bound To | gi\|295321916 |
| F-Actin | |
| Chain I, Model Of Alpha-Actinin Ch1 Bound To | gi\|295321917 |
| F-Actin | |
| Chain J, Model Of Alpha-Actinin Ch1 Bound To | gi\|295321918 |
| F-Actin | |
| Unknown (protein for IMAGE: 3538275) | gi\|16924319 |
| [Homo sapiens] | |
| unnamed protein product [Homo sapiens] | gi\|194385944 |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.5624 | −.0048 | −5 | 322 | 329 | IIAPPERK |
| 976.4483 | 976.4526 | .0043 | 4 | 12 | 21 | AGFAGDDAPR |
| 976.4483 | 976.4526 | .0043 | 4 | 12 | 21 | AGFAGDDAPR |
| 1036.6514 | 1036.6522 | .0008 | 1 | 320 | 328 | IKIIAPPER |
| 1132.527 | 1132.53 | .003 | 3 | 190 | 199 | GYSFTTTAER |
| 1132.527 | 1132.53 | .003 | 3 | 190 | 199 | GYSFTTTAER |
| 1198.7056 | 1198.7052 | −.0004 | 0 | 22 | 32 | AVFPSIVG |
| | | | | | | RPR |
| 1198.7056 | 1198.7052 | −.0004 | 0 | 22 | 32 | AVFPSIVG |
| | | | | | | RPR |
| 1516.7028 | 1516.7002 | −.0026 | −2 | 353 | 365 | QEYDESGP |
| | | | | | | SIVHR |
| 1516.7028 | 1516.7002 | −.0026 | −2 | 353 | 365 | QEYDESGP |
| | | | | | | SIVHR |
| 1629.8231 | 1629.8131 | −.01 | −6 | 190 | 203 | GYSFTTTAERE |
| | | | | | | IVR |
| 1639.8361 | 1639.8319 | −.0042 | −3 | 171 | 184 | LDLAGRDLTDY |
| | | | | | | LMK |
| 1790.892 | 1790.8784 | −.0136 | −8 | 232 | 247 | SYELPDGQVITI |
| | | | | | | GNER |
| 1790.892 | 1790.8784 | −.0136 | −8 | 232 | 247 | SYELPDGQVITI |
| | | | | | | GNER |
| 1954.0645 | 1954.0728 | .0083 | 4 | 89 | 106 | VAPEEHPVLLT |
| | | | | | | EAPLNPK |
| 2231.0649 | 2231.0669 | .002 | 1 | 285 | 305 | DLYANTVLSGG |
| | | | | | | TTMYPGI |
| | | | | | | ADR |

Protein Group

| | |
|---|---|
| RecName: Full = Actin, cytoplasmic 2; AltName: | gi\|54036678 |
| Full = Gamma-actin; Contains: | |
| RecName: Full = Actin, cytoplasmic 2, | |
| N-terminally processed | |
| ACTG1 protein [Homo sapiens] | gi\|16041790 |
| ACTG1 protein [Homo sapiens] | gi\|15929094 |
| ACTG1 protein [Homo sapiens] | gi\|12804935 |
| ACTG1 protein [Homo sapiens] | gi\|17511847 |
| ACTG1 protein [Homo sapiens] | |
| Actin, beta [Homo sapiens] | gi\|15426536 |
| Actin, beta [Homo sapiens] | gi\|12654911 |
| Actin, beta [Homo sapiens] | gi\|12803203 |
| Actin, beta [Homo sapiens] | gi\|13279023 |
| Actin, beta [Homo sapiens] | gi\|15928803 |
| Actin, gamma 1 [Homo sapiens] | gi\|31565757 |
| Actin, gamma 1 [Homo sapiens] | gi\|15082291 |
| Actin, gamma 1 [Homo sapiens] | gi\|12653055 |
| Actin, gamma 1 [Homo sapiens] | gi\|13938581 |
| Actin, gamma 1 [Homo sapiens] | gi\|15990564 |
| Actin, gamma 1 [Homo sapiens] | gi\|15012187 |
| Actin, gamma 1 [Homo sapiens] | gi\|14602652 |
| RecName: Full = Actin, cytoplasmic 1; AltName: | gi\|46397333 |
| Full = Beta-actin; Contains: RecName: | |
| Full = Actin, cytoplasmic 1, N-terminally | |
| processed | |
| actin, beta [Homo sapiens] | gi\|45934426 |
| actin, cytoplasmic 1 [Homo sapiens] | gi\|4501885 |
| actin, cytoplasmic 2 [Homo sapiens] | gi\|316659409 |
| actin, cytoplasmic 2 [Homo sapiens] | gi\|4501887 |
| actin, cytoplasmic 2 [Homo sapiens] | |
| actin, gamma 1 [Homo sapiens] | gi\|54696574 |
| cytoplasmic beta actin [Homo sapiens] | gi\|177968 |
| gamma-actin [Homo sapiens] | gi\|178043 |
| hCG15971, isoform CRA_a [Homo sapiens] | gi\|119607749 |
| hCG15971, isoform CRA_a [Homo sapiens] | gi\|119607747 |
| unknown [Homo sapiens] | gi\|30172702 |
| unnamed protein product [Homo sapiens] | gi\|284806909 |
| unnamed protein product [Homo sapiens] | gi\|283915537 |
| unnamed protein product [Homo sapiens] | gi\|229610526 |
| unnamed protein product [Homo sapiens] | gi\|213500113 |
| unnamed protein product [Homo sapiens] | gi\|210029289 |
| unnamed protein | |
| product [Homo | |
| sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.5624 | −.0048 | −5 | 329 | 336 | IIAPPERK |
| 976.4483 | 976.4526 | .0043 | 4 | 19 | 28 | AGFAGDDAPR |
| 976.4483 | 976.4526 | .0043 | 4 | 19 | 28 | AGFAGDDAPR |
| 1036.6514 | 1036.6522 | .0008 | 1 | 327 | 335 | IKIIAPPER |
| 1132.527 | 1132.53 | .003 | 3 | 197 | 206 | GYSFTTTAER |
| 1132.527 | 1132.53 | .003 | 3 | 197 | 206 | GYSFTTTAER |
| 1198.7056 | 1198.7052 | −.0004 | 0 | 29 | 39 | AVFPSIVGRPR |
| 1198.7056 | 1198.7052 | −.0004 | 0 | 29 | 39 | AVFPSIVGRPR |
| 1516.7028 | 1516.7002 | −.0026 | −2 | 360 | 372 | QEYDESGPSIV |
| | | | | | | HR |
| 1516.7028 | 1516.7002 | −.0026 | −2 | 360 | 372 | QEYDESGPSIV |
| | | | | | | HR |
| 1629.8231 | 1629.8131 | −.01 | −6 | 197 | 210 | GYSFTTTAERE |
| | | | | | | IVR |
| 1639.8361 | 1639.8319 | −.0042 | −3 | 178 | 191 | LDLAGRDLTDY |
| | | | | | | LMK |
| 1790.892 | 1790.8784 | −.0136 | −8 | 239 | 254 | SYELPDGQVITI |
| | | | | | | GNER |
| 1790.892 | 1790.8784 | −.0136 | −8 | 239 | 254 | SYELPDGQVITI |
| | | | | | | GNER |
| 1954.0645 | 1954.0728 | .0083 | 4 | 96 | 113 | VAPEEHPVLLT |
| | | | | | | EAPLNPK |
| 2231.0649 | 2231.0669 | .002 | 1 | 292 | 312 | DLYANTVLSGG |
| | | | | | | TTMYPGI |
| | | | | | | ADR |

| | |
|---|---|
| POTE ankyrin domain family member E | gi\|134133226 |
| [Homo sapiens] | |

Protein Group

| | |
|---|---|
| Actin, beta [Homo sapiens] | gi\|134133226 |
| | gi\|16359158 |
| POTE ankyrin domain family member F [Homo sapiens] | gi\|153791352 |
| POTE-2 alpha-actin [Homo sapiens] | gi\|145309046 |
| PS1TP5-binding protein 1 [Homo sapiens] | gi\|93211215 |
| RecName: Full = POTE ankyrin domain family member | gi\|166898073 |
| E; AltName: Full = ANKRD26-like family | |
| C member 1A; AltName: Full = Prostate, | |
| ovary, testis-expressed | |
| protein | |
| on chromosome 2; Short = POTE-2 | |
| RecName: Full = POTE ankyrin domain family member | gi\|215273934 |
| F; AltName: Full = ANKRD26-like family | |
| C member 1B; AltName: Full = Chimeric | |
| POTE-actin | |
| protein | |
| beta actin variant [Homo sapiens] | gi\|62897671 |
| beta actin variant [Homo sapiens] | gi\|62897409 |
| beta actin variant [Homo sapiens] | |
| beta actin variant [Homo sapiens] | gi\|62897625 |
| gamma-actin [Homo sapiens] | gi\|28339 |
| mutant beta-actin (beta′-actin) [Homo sapiens] | gi\|28336 |
| unnamed protein product [Homo sapiens] | gi\|194375299 |
| unnamed protein product [Homo sapiens] | gi\|158257306 |
| unnamed protein product [Homo sapiens] | gi\|66348792 |
| unnamed protein product [Homo sapiens] | gi\|28252 |

Peptide Information

| | | | | Start | End | |
|---|---|---|---|---|---|---|
| Calc. Mass | Obsrv. Mass | ±da | ±ppm | Seq. | Seq. | Sequence |
| 976.4483 | 976.4526 | .0043 | 4 | 719 | 728 | AGFAGDDAPR |
| 1198.7056 | 1198.7052 | −.0004 | 0 | 729 | 739 | AVFPSIVGRPR |
| 1516.7028 | 1516.7002 | −.0026 | −2 | 1060 | 1072 | QEYDESGPSIVHR |
| 1790.892 | 1790.8784 | −.0136 | −8 | 939 | 954 | SYELPDGQVITIGN |
| | | | | | | ER |

| | |
|---|---|
| Chain C, The Structure Of The Human Rnase H2 | gi\|312208155 |
| Complex Defines Key Interaction Interfaces | |
| Relevant To Enzyme Function And Human | |
| Disease | |

Protein Group

| | |
|---|---|
| AYP1 [Homo sapiens] | gi\|28628365 |
| AYP1 [Homo sapiens] | gi\|28628363 |
| AYP1 protein, isoform CRA_a [Homo sapiens] | gi\|119594839 |
| AYP1 protein, isoform CRA_b [Homo sapiens] | gi\|119594841 |
| AYP1 protein, isoform CRA_b [Homo sapiens] | gi\|119594840 |
| Chain F, The Structure Of The Human Rnase H2 | gi\|312208158 |
| Complex Defines Key Interaction Interfaces Relevant | |
| To Enzyme Function And Human | |
| Disease | |
| RNase H1 small subunit [Homo sapiens] | gi\|19421812 |
| RecName: Full = Ribonuclease H2 subunit C; | gi\|74730607 |
| Short = RNase H2 subunit C; AltName: | |
| Full = Aicardi-Goutieres syndrome 3 protein; | |
| Short = AGS3; AltName: Full = RNase H1 | |
| small subunit; | |
| AltName: Full = Ribonuclease HI subunit C | |
| Ribonuclease H2, subunit C [Homo sapiens] | gi\|23270834 |
| ribonuclease H2 subunit C [Homo sapiens] | gi\|38176285 |
| unnamed protein product [Homo sapiens] | gi\|194387324 |
| unnamed protein product [Homo sapiens] | gi\|10436946 |

Peptide Information

| Calc. | Obsrv. | | | Start | End | |
|---|---|---|---|---|---|---|
| Mass | Mass | ±da | ±ppm | Seq. | Seq. | Sequence |
| 1516.6809 | 1516.7002 | .0193 | 13 | 1 | 13 | MESGDEA |
| | | | | | | AIERHR |

| | |
|---|---|
| baculoviral IAP repeat-containing | gi\|119595708 |
| 7 (livin), isoform | |
| CRA_a [Homo sapiens] | gi\|15680241 |

Protein Group

| | |
|---|---|
| Baculoviral IAP repeat-containing 7 [Homo sapiens] | |
| LIVIN [Homo sapiens] | gi\|37182790 |
| LIVIN [Homo sapiens] | gi\|37182788 |
| RecName: Full = Baculoviral IAP repeat-containing | gi\|21759008 |
| protein 7; AltName: Full = Kidney inhibitor of apoptosis | |
| protein; Short = KIAP; AltName: Full = Livin; | |
| AltName: Full = Melanoma inhibitor of | |
| apoptosis protein; Short = ML-IAP; AltName: | |
| Full = RING finger protein 50 | |
| baculoviral IAP repeat-containing 7 (livin) [Homo | gi\|13560105 |
| sapiens] | |
| baculoviral IAP repeat-containing 7 (livin) [Homo | gi\|13560104 |
| sapiens] | |
| baculoviral IAP repeat-containing 7 (livin), isoform | gi\|119595709 |
| CRA_b [Homo sapiens] | |
| baculoviral IAP repeat-containing 7 [Homo sapiens] | gi\|41387695 |
| baculoviral IAP repeat-containing protein 7 isoform | gi\|21536421 |
| alpha [Homo sapiens] | |
| baculoviral IAP repeat-containing protein 7 isoform beta | gi\|11545910 |
| [Homo sapiens] | |
| inhibitor of apoptosis [Homo sapiens] | gi\|13785205 |
| inhibitor of apoptosis [Homo sapiens] | gi\|13785204 |
| inhibitor of apoptosis [Homo sapiens] | |
| inhibitor of apoptosis protein KIAP [Homo sapiens] | gi\|11545503 |
| livin inhibitor-of-apotosis [Homo sapiens] | gi\|11245453 |

Peptide Information

| Calc. | Obsrv. | | | Start | End | |
|---|---|---|---|---|---|---|
| Mass | Mass | ±da | ±ppm | Seq. | Seq. | Sequence |
| 1790.8206 | 1790.8784 | .0578 | 32 | 13 | 29 | GPQPSHWA |
| | | | | | | AGDGPTQ |
| | | | | | | ER |

| | |
|---|---|
| hsp90 co-chaperone Cdc37-like | gi\|66773038 |
| 1 [Homo sapiens] | |

Protein Group

| | |
|---|---|
| CDC37 cell division cycle 37 homolog | gi\|119579186 |
| (S. cerevisiae)-like 1 [Homo sapiens] | |
| Cell division cycle 37 homolog (S. cerevisiae)-like 1 | gi\|15559546 |
| [Homo sapiens] | |
| RecName: Full = Hsp90 co-chaperone Cdc37-like 1; | gi\|182705252 |
| AltName: Full = Hsp90-associating relative of Cdc37 | |
| cell division cycle 37 homolog (S. cerevisiae)-like 1 | gi\|57208646 |
| [Homo sapiens] | |
| cell division cycle 37 homolog (S. cerevisiae)-like 1 | gi\|10696978 |
| [Homo sapiens] | |
| unnamed protein product [Homo sapiens] | gi\|7020874 |

| | | | |
|---|---|---|---|
| | 75/C25 | | Instr./Gel |
| | | | Origin |
| | [1] Sample Project | | Instrument |
| | 20110504 | | Sample |
| | | | Name |
| Protein Name- | KH18 (SEQ ID NO. KH18) | Accession No. | |

| | |
|---|---|
| unnamed protein product [Homo sapiens] | gi\|194376311 |

Peptide Information

| | | | | Start | End | |
|---|---|---|---|---|---|---|
| Calc. Mass | Obsrv. Mass | ±da | ±ppm | Seq. | Seq. | Sequence |
| 2066.6760 | 2057.6322 | −.0055 | −4 | 211 | 229 | IIAPPERK |
| 2135.2369 | 2126.3699 | −.0059 | −4 | 215 | 233 | AGFAGDDAPR |
| 2203.7978 | 2195.1077 | −.0063 | −4 | 219 | 238 | AGFAGDDAPR |
| 2272.3587 | 2263.8454 | −.0067 | −5 | 223 | 243 | IKIIAPPER |
| 2340.920 | 2332.58 | −.007 | −5 | 227 | 247 | GYSFTTTAER |
| 2409.480 | 2401.32 | −.007 | −5 | 231 | 252 | GYSFTTTAER |
| 2478.0414 | 2470.0585 | −.0079 | −5 | 235 | 256 | AVFPSIVGRPR |
| 2546.6023 | 2538.7963 | −.0083 | −6 | 239 | 261 | AVFPSIVGRPR |
| 2615.163 | | | | | | |
| 2607.5340 | | | | | | |
| −0.00869 | | | | | | |

−
−6.04545 243.4587 265.6219
−2683.724 2676.272
0.00909
6.34091 247.4742 270.2324

| | | | | | | |
|---|---|---|---|---|---|---|
| 2752.2850 | 2745.0094 | −.0095 | −7 | 251 | 275 | QEYDESGPSIVHR |
| 2820.8459 | 2813.7471 | −.0099 | −7 | 256 | 279 | QEYDESGPSIVHR |
| 2889.4068 | 2882.4848 | −.01 | −7 | 260 | 284 | GYSFTTTAEREIVR |
| 2957.9677 | 2951.2226 | −.0107 | −8 | 264 | 289 | LDLAGRDLTDYLMK |
| 3026.529 | 3019.9603 | −.0111 | −8 | 268 | 293 | SYELPDGQVITIGNER |
| 3095.089 | 3088.6980 | −.0115 | −8 | 272 | 298 | SYELPDGQVITIGNER |
| 3163.6504 | 3157.4357 | −.0119 | −8 | 276 | 303 | VAPEEHPVLLTEAPLNPK |
| 3232.2113 | 3226.1734 | −.012 | −9 | 280 | 307 | DLYANTVLSGGTTMYPGI |
| | | | | | | ADR |

| | |
|---|---|
| unnamed protein product [Homo sapiens] | gi\|194388065 |

Peptide Information

| | Obsrv. | | | Start | End | |
|---|---|---|---|---|---|---|
| Calc. Mass | Mass | ±da | ±ppm | Seq. | Seq. | Sequence |
| 2107.5744 | 2107.5690 | −.0054 | −4 | 218 | 236 | IIAPPERK |
| 2189.0128 | 2189.0070 | −.0058 | −4 | 223 | 241 | AGFAGDDAPR |
| 2270.4513 | 2270.4450 | −.0062 | −4 | 227 | 246 | AGFAGDDAPR |
| 2351.8897 | 2351.8831 | −.0067 | −5 | 232 | 251 | IKIIAPPER |
| 2433.328 | 2433.32 | −.007 | −5 | 236 | 257 | GYSFTTTAER |
| 2514.767 | 2514.76 | −.008 | −5 | 240 | 262 | GYSFTTTAER |
| 2596.2051 | 2596.1972 | −.0080 | −6 | 245 | 267 | AVFPSIVGRPR |
| 2677.6436 | 2677.6352 | −.0084 | −6 | 249 | 272 | AVFPSIVGRPR |
| 2759.0821 | 2759.0732 | −.0089 | −6 | 253 | 277 | QEYDESGPSIVHR |
| 2840.5205 | 2840.5112 | −.0093 | −6 | 258 | 282 | QEYDESGPSIVHR |
| 2921.9590 | 2921.9493 | −.01 | −7 | 262 | 287 | GYSFTTTAEREIVR |
| 3003.3975 | 3003.3873 | −.0102 | −7 | 267 | 292 | LDLAGRDLTDYLMK |
| 3084.836 | 3084.8253 | −.0106 | −7 | 271 | 297 | SYELPDGQVITIGN |
| | | | | | | ER |
| 3166.274 | 3166.2633 | −.0110 | −8 | 275 | 302 | SYELPDGQVITIGN |
| | | | | | | ER |
| 3247.7128 | 3247.7014 | −.0115 | −8 | 280 | 307 | VAPEEHPVLLTEA |
| | | | | | | PLNPK |
| 3329.1513 | 3329.1394 | −.012 | −8 | 284 | 313 | DLYANTVLSGGTT |
| | | | | | | MYPGI |
| | | | | | | ADR |

| | |
|---|---|
| ACTB protein [Homo sapiens] | gi\|15277504 |

Peptide Information

| | | | | Start | End | |
|---|---|---|---|---|---|---|
| Calc. Mass | Obsrv. Mass | ±da | ±ppm | Seq. | Seq. | Sequence |
| 2056.9512 | 2056.9462 | −.0050 | −3 | 216 | 234 | IIAPPERK |
| 2130.2773 | 2130.2719 | −.0054 | −4 | 221 | 239 | AGFAGDDAPR |
| 2203.603 | 2203.598 | −0.00576 | −3.82258 | 224.8883 | 243.4938 | |
| 2276.93 | 2276.923 | −0.00615 | −4.09677 | 229.1396 | 248.3828 | |
| 2350.2558 | 2350.2492 | −.0065 | −4 | 233 | 253 | AGFAGDDAPR |
| 2423.5819 | 2423.5750 | −.0069 | −5 | 238 | 258 | IKIIAPPER |
| 2496.908 | 2496.90 | −.007 | −5 | 242 | 263 | GYSFTTTAER |
| 2570.234 | 2570.23 | −.008 | −5 | 246 | 268 | GYSFTTTAER |
| 2643.5603 | 2643.5523 | −.0081 | −5 | 250 | 273 | AVFPSIVGRPR |
| 2716.8865 | 2716.8780 | −.0084 | −6 | 255 | 278 | AVFPSIVGRPR |
| 2790.2126 | 2790.2038 | −.0088 | −6 | 259 | 283 | QEYDESGPSIV |
| | | | | | | HR |
| 2863.5388 | 2863.5296 | −.0092 | −6 | 263 | 287 | QEYDESGPSIV |
| | | | | | | HR |
| 2936.8649 | 2936.8553 | −.01 | −7 | 267 | 292 | GYSFTTTAERE |
| | | | | | | IVR |
| 3010.1911 | 3010.1811 | −.0100 | −7 | 272 | 297 | LDLAGRDLTDY |
| | | | | | | LMK |
| 3083.517 | 3083.5069 | −.0103 | −7 | 276 | 302 | SYELPDGQVITI |
| | | | | | | GNER |
| 3156.843 | 3156.8326 | −.0107 | −7 | 280 | 307 | SYELPDGQVITI |
| | | | | | | GNER |
| 3230.1695 | 3230.1584 | −.0111 | −8 | 284 | 312 | VAPEEHPVLLT |
| | | | | | | EAPLNPK |
| 3303.4956 | 3303.4841 | −.011 | −8 | 289 | 317 | DLYANTVLSGG |
| | | | | | | TTMYPGI |
| | | | | | | ADR |

| | |
|---|---|
| actin, beta [Homo sapiens] | gi\|14250401 |

Protein Group

| | |
|---|---|
| Chain A, Model Of Actin-Fimbrin Abd3 Complex | gi\|168177284 |
| Chain A, Model Of Alpha-Actinin Ch2 Bound To | gi\|295321909 |
| F-Actin | |
| Chain B, Model Of Alpha-Actinin Ch2 Bound To | gi\|295321910 |
| F-Actin | |
| Chain C, Model Of Alpha-Actinin Ch2 Bound To | gi\|295321911 |
| F-Actin | |
| Chain D, Model Of Alpha-Actinin Ch2 Bound To | gi\|295321912 |
| F-Actin | |
| Chain E, Model Of Alpha-Actinin Ch2 Bound To | gi\|295321913 |
| F-Actin | |
| Chain F, Model Of Alpha-Actinin Ch2 Bound To | gi\|295321914 |
| F-Actin | |
| Chain G, Model Of Alpha-Actinin Ch2 Bound To | gi\|295321915 |
| F-Actin | |
| Chain H, Model Of Alpha-Actinin Ch2 Bound To F- | gi\|295321916 |
| Actin | |
| Chain I, Model Of Alpha-Actinin Ch2 Bound To F-Actin | gi\|295321917 |
| Chain J, Model Of Alpha-Actinin Ch2 Bound To F-Actin | gi\|295321918 |
| Unknown (protein for IMAGE: 3538276) | gi\|16924319 |
| [Homo sapiens] | |
| unnamed protein product [Homo sapiens] | gi\|194385944 |

Peptide Information

| | | | | Start | End | |
|---|---|---|---|---|---|---|
| Calc. Mass | Obsrv. Mass | ±da | ±ppm | Seq. | Seq. | Sequence |
| 2044.6767 | 2044.6713 | −.0053 | −4 | 222 | 239 | IIAPPERK |
| 2112.7123 | 2112.7065 | −.0057 | −4 | 226 | 244 | AGFAGDDAPR |
| 2180.7479 | 2180.7417 | −.0061 | −4 | 230 | 248 | AGFAGDDAPR |
| 2248.7835 | 2248.7769 | −.0065 | −4 | 233 | 252 | IKIIAPPER |
| 2316.819 | 2316.81 | −.007 | −5 | 237 | 257 | GYSFTTTAER |
| 2384.855 | 2384.85 | −.007 | −5 | 241 | 261 | GYSFTTTAER |
| 2452.89 | 2452.883 | −0.00774 | −5.35368 | 244.5516 | 265.3232 | |
| 2520.926 | 2520.918 | −0.00814 | −5.64737 | 248.3132 | 269.6763 | |
| 2588.9614 | 2588.9529 | −.0085 | −6 | 252 | 274 | AVFPSIVGRPR |
| 2656.9970 | 2656.9881 | −.0089 | −6 | 256 | 278 | AVFPSIVGRPR |
| 2725.0326 | 2725.0233 | −.0093 | −7 | 260 | 283 | QEYDESGPSIVHR |
| 2793.0682 | 2793.0585 | −.0097 | −7 | 263 | 287 | QEYDESGPSIVHR |
| 2861.1038 | 2861.0937 | −.01 | −7 | 267 | 291 | GYSFTTTAEREIVR |
| 2929.1394 | 2929.1289 | −.0105 | −7 | 271 | 296 | LDLAGRDLTDYLMK |
| 2997.175 | 2997.1641 | −.0109 | −8 | 275 | 300 | SYELPDGQVITIGN |
| | | | | | | ER |
| 3065.211 | 3065.1993 | −.0113 | −8 | 278 | 305 | SYELPDGQVITIGN |
| | | | | | | ER |
| 3133.2462 | 3133.2345 | −.0117 | −8 | 282 | 309 | VAPEEHPVLLTEA |
| | | | | | | PLNPK |
| 3201.2818 | 3201.2697 | −.012 | −9 | 286 | 313 | DLYANTVLSGGTT |
| | | | | | | MYPGI |
| | | | | | | ADR |

| | |
|---|---|
| RecName: Full = Actin, cytoplasmic 3; AltName: | gi\|54036678 |
| Full = Gamma-actin; Contains: | |
| RecName: Full = Actin, cytoplasmic | |
| 3, N-terminally processed | |

Protein Group

| | |
|---|---|
| ACTG1 protein [Homo sapiens] | gi\|16041790 |
| ACTG1 protein [Homo sapiens] | gi\|15929094 |
| ACTG1 protein [Homo sapiens] | gi\|12804935 |
| ACTG1 protein [Homo sapiens] | gi\|17511847 |
| ACTG1 protein [Homo sapiens] | |
| Actin, beta [Homo sapiens] | gi\|15426536 |
| Actin, beta [Homo sapiens] | gi\|12654911 |
| Actin, beta [Homo sapiens] | gi\|12803203 |
| Actin, beta [Homo sapiens] | gi\|13279023 |
| Actin, beta [Homo sapiens] | gi\|15928803 |
| Actin, gamma 1 [Homo sapiens] | gi\|31565757 |
| Actin, gamma 1 [Homo sapiens] | gi\|15082291 |
| Actin, gamma 1 [Homo sapiens] | gi\|12653055 |
| Actin, gamma 1 [Homo sapiens] | gi\|13938581 |
| Actin, gamma 1 [Homo sapiens] | gi\|15990564 |
| Actin, gamma 1 [Homo sapiens] | gi\|15012187 |
| Actin, gamma 1 [Homo sapiens] | gi\|14602652 |
| RecName: Full = Actin, cytoplasmic 2; AltName: | gi\|46397333 |
| Full = Beta-actin; Contains: RecName: | |
| Full = Actin, cytoplasmic 2, N-terminally | |
| processed | |
| actin, beta [Homo sapiens] | gi\|45934426 |
| actin, cytoplasmic 1 [Homo sapiens] | gi\|4501885 |
| actin, cytoplasmic 2 [Homo sapiens] | gi\|316659409 |
| actin, cytoplasmic 2 [Homo sapiens] | gi\|4501887 |
| actin, cytoplasmic 2 [Homo sapiens] | |
| actin, gamma 2 [Homo sapiens] | gi\|54696574 |
| cytoplasmic beta actin [Homo sapiens] | gi\|177968 |
| gamma-actin [Homo sapiens] | gi\|178043 |
| hCG15971, isoform CRA_a [Homo sapiens] | gi\|119607749 |
| hCG15971, isoform CRA_a [Homo sapiens] | gi\|119607747 |
| unknown [Homo sapiens] | gi\|30172702 |
| unnamed protein product [Homo sapiens] | gi\|284806909 |
| unnamed protein product [Homo sapiens] | gi\|283915537 |
| unnamed protein product [Homo sapiens] | gi\|229610526 |
| unnamed protein product [Homo sapiens] | gi\|213500113 |
| unnamed protein product [Homo sapiens] | gi\|210029289 |
| unnamed protein | |
| product [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 2107.5744 | 2107.5690 | −.0054 | −4 | 235 | 253 | IIAPPERK |
| 2189.0128 | 2189.0070 | −.0058 | −4 | 240 | 258 | AGFAGDDAPR |
| 2270.4513 | 2270.4450 | −.0062 | −4 | 245 | 264 | AGFAGDDAPR |
| 2351.8897 | 2351.8831 | −.0067 | −5 | 249 | 269 | IKIIAPPER |
| 2433.328 | 2433.32 | −.007 | −5 | 254 | 275 | GYSFTTTAER |
| 2514.767 | 2514.76 | −.008 | −5 | 259 | 280 | GYSFTTTAER |
| 2596.2051 | 2596.1972 | −.0080 | −6 | 264 | 286 | AVFPSIVGRPR |
| 2677.6436 | 2677.6352 | −.0084 | −6 | 269 | 291 | AVFPSIVGRPR |
| 2759.0821 | 2759.0732 | −.0089 | −6 | 273 | 297 | QEYDESGPSIVHR |
| 2840.5205 | 2840.5112 | −.0093 | −6 | 278 | 302 | QEYDESGPSIVHR |
| 2921.9590 | 2921.9493 | −.01 | −7 | 283 | 308 | GYSFTTTAEREIVR |
| 3003.3975 | 3003.3873 | −.0102 | −7 | 288 | 313 | LDLAGRDLTDYLMK |
| 3084.836 | 3084.8253 | −.0106 | −7 | 293 | 319 | SYELPDGQVITIGN |
| | | | | | | ER |
| 3166.274 | 3166.2633 | −.0110 | −8 | 297 | 324 | SYELPDGQVITIGN |
| | | | | | | ER |
| 3247.7128 | 3247.7014 | −.0115 | −8 | 302 | 330 | VAPEEHPVLLTEA |
| | | | | | | PLNPK |
| 3329.1513 | 3329.1394 | −.012 | −8 | 307 | 335 | DLYANTVLSGGTT |
| | | | | | | MYPGI |
| | | | | | | ADR |

| | |
|---|---|
| POTE ankyrin domain family member E | gi\|134133226 |
| [Homo sapiens] | |

Protein Group

| | |
|---|---|
| Actin, beta [Homo sapiens] | gi\|16359158 |
| POTE ankyrin domain family member F [Homo sapiens] | gi\|153791352 |
| POTE-3 alpha-actin [Homo sapiens] | gi\|145309046 |
| PS1TP5-binding protein 2 [Homo sapiens] | gi\|93211215 |
| RecName: Full = POTE ankyrin domain family member | gi\|166898073 |
| E; AltName: Full = ANKRD26-like family C | |
| member 2A; AltName: Full = Prostate, ovary, | |
| testis-expressed | |
| protein | |
| on chromosome 2; | |
| Short = POTE-3 | |
| RecName: Full = POTE ankyrin domain family member | gi\|215273934 |
| F; AltName: Full = ANKRD26-like family C | |
| member 2B; AltName: Full = Chimeric POTE- | |
| actin | |
| protein | |
| beta actin variant [Homo sapiens] | gi\|62897671 |
| beta actin variant [Homo sapiens] | gi\|62897409 |
| beta actin variant [Homo sapiens] | |
| beta actin variant [Homo sapiens] | gi\|62897625 |
| gamma-actin [Homo sapiens] | gi\|28339 |
| mutant beta-actin (beta′-actin) [Homo sapiens] | gi\|28336 |
| unnamed protein product [Homo sapiens] | gi\|194375299 |
| unnamed protein product [Homo sapiens] | gi\|158257306 |
| unnamed protein product [Homo sapiens] | gi\|66348792 |
| unnamed protein product [Homo sapiens] | gi\|28252 |

Peptide Information

| Calc. | Obsrv. | | | Start | End | |
|---|---|---|---|---|---|---|
| Mass | Mass | ±da | ±ppm | Seq. | Seq. | Sequence |
| 2061.0193 | 2061.0022 | −.0171 | −11 | 1110 | 1126 | AGFAGD |
| | | | | | | DAPR |
| 2337.1521 | 2337.1294 | −.0226 | −15 | 1209 | 1227 | AVFPSI |
| | | | | | | VGRPR |
| 2613.2849 | 2613.2567 | −.0282 | −19 | 1308 | 1328 | QEYDESG |
| | | | | | | PSIVHR |

| | |
|---|---|
| 76/D1 | Instr./Gel Origin |
| [1] Sample Project | Instrument |
| 20110503 | Sample |
| | Name |

| | |
|---|---|
| Protein Name | Accession No. |
| unnamed protein product [Homo | gi\|194388064 |
| sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.5703 | .0031 | 3 | 310 | 317 | IIAPPERK |
| 976.4483 | 976.4528 | .0045 | 5 | 19 | 28 | AGFAGDDAPR |
| 976.4483 | 976.4528 | .0045 | 5 | 19 | 28 | AGFAGDDAPR |
| 1132.527 | 1132.526 | −.001 | −1 | 178 | 187 | GYSFTTTAER |
| 1132.527 | 1132.526 | −.001 | −1 | 178 | 187 | GYSFTTTAER |
| 1198.7056 | 1198.7018 | −.0038 | −3 | 29 | 39 | AVFPSIVGRPR |
| 1198.7056 | 1198.7018 | −.0038 | −3 | 29 | 39 | AVFPSIVGRPR |
| 1354.6234 | 1354.6217 | −.0017 | −1 | 51 | 62 | DSYVGDEAQSKR |
| 1516.7028 | 1516.6896 | −.0132 | −9 | 341 | 353 | QEYDESGPSIVHR |
| 1516.7028 | 1516.6896 | −.0132 | −9 | 341 | 353 | QEYDESGPSIVHR |
| 1629.8231 | 1629.8079 | −.0152 | −9 | 178 | 191 | GYSFTTTAEREIVR |
| 1639.8361 | 1639.8278 | −.0083 | −5 | 159 | 172 | LDLAGRDLTDYLMK |
| 1790.892 | 1790.8685 | −.0235 | −13 | 220 | 235 | SYELPDGQVITIGN |
| | | | | | | ER |
| 1790.892 | 1790.8685 | −.0235 | −13 | 220 | 235 | SYELPDGQVITIGN |
| | | | | | | ER |
| 1954.0645 | 1954.0369 | −.0276 | −14 | 96 | 113 | VAPEEHPVLLTEA |
| | | | | | | PLNPK |
| 2231.0649 | 2231.0745 | .0096 | 4 | 273 | 293 | DLYANTVLSGGTT |
| | | | | | | MYPGI |
| | | | | | | ADR |

| | |
|---|---|
| ACTB protein [Homo | gi\|15277503 |
| sapiens] | |

Peptide Information

| | Obsrv. | | | Start | | |
|---|---|---|---|---|---|---|
| Calc. Mass | Mass | ±da | ±ppm | Seq. | End Seq. | Sequence |
| 923.5672 | 923.5703 | .0031 | 3 | 314 | 321 | IIAPPERK |
| 976.4483 | 976.4528 | .0045 | 5 | 4 | 13 | AGFAGDDAPR |
| 976.4483 | 976.4528 | .0045 | 5 | 4 | 13 | AGFAGDDAPR |
| 1132.527 | 1132.526 | −.001 | −1 | 182 | 191 | GYSFTTTAER |
| 1132.527 | 1132.526 | −.001 | −1 | 182 | 191 | GYSFTTTAER |
| 1198.7056 | 1198.7018 | −.0038 | −3 | 14 | 24 | AVFPSIVGRPR |
| 1198.7056 | 1198.7018 | −.0038 | −3 | 14 | 24 | AVFPSIVGRPR |
| 1354.6234 | 1354.6217 | −.0017 | −1 | 36 | 47 | DSYVGDEAQS |
| | | | | | | KR |
| 1516.7028 | 1516.6896 | −.0132 | −9 | 345 | 357 | QEYDESGPSIV |
| | | | | | | HR |
| 1516.7028 | 1516.6896 | −.0132 | −9 | 345 | 357 | QEYDESGPSIV |
| | | | | | | HR |
| 1629.8231 | 1629.8079 | −.0152 | −9 | 182 | 195 | GYSFTTTAERE |
| | | | | | | IVR |
| 1639.8361 | 1639.8278 | −.0083 | −5 | 163 | 176 | LDLAGRDLTDY |
| | | | | | | LMK |
| 1790.892 | 1790.8685 | −.0235 | −13 | 224 | 239 | SYELPDGQVITI |
| | | | | | | GNER |
| 1790.892 | 1790.8685 | −.0235 | −13 | 224 | 239 | SYELPDGQVITI |
| | | | | | | GNER |
| 1954.0645 | 1954.0369 | −.0276 | −14 | 81 | 98 | VAPEEHPVLLT |
| | | | | | | EAPLNPK |
| 2231.0649 | 2231.0745 | .0096 | 4 | 277 | 297 | DLYANTVLSGG |
| | | | | | | TTMYPGI |
| | | | | | | ADR |

| | |
|---|---|
| unnamed protein product [Homo sapiens] | gi\|194385944 |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.5703 | .0031 | 3 | 305 | 312 | IIAPPERK |
| 976.4483 | 976.4528 | .0045 | 5 | 19 | 28 | AGFAGDDAPR |
| 976.4483 | 976.4528 | .0045 | 5 | 19 | 28 | AGFAGDDAPR |
| 1132.527 | 1132.526 | −.001 | −1 | 173 | 182 | GYSFTTTAER |
| 1132.527 | 1132.526 | −.001 | −1 | 173 | 182 | GYSFTTTAER |
| 1198.7056 | 1198.7018 | −.0038 | −3 | 29 | 39 | AVFPSIVGRPR |
| 1198.7056 | 1198.7018 | −.0038 | −3 | 29 | 39 | AVFPSIVGRPR |
| 1354.6234 | 1354.6217 | −.0017 | −1 | 51 | 62 | DSYVGDEAQSKR |
| 1516.7028 | 1516.6896 | −.0132 | −9 | 336 | 348 | QEYDESGPSIVHR |
| 1516.7028 | 1516.6896 | −.0132 | −9 | 336 | 348 | QEYDESGPSIVHR |
| 1629.8231 | 1629.8079 | −.0152 | −9 | 173 | 186 | GYSFTTTAEREIVR |
| 1639.8361 | 1639.8278 | −.0083 | −5 | 154 | 167 | LDLAGRDLTDYLMK |
| 1790.892 | 1790.8685 | −.0235 | −13 | 215 | 230 | SYELPDGQVITIGN |
| | | | | | | ER |
| 1790.892 | 1790.8685 | −.0235 | −13 | 215 | 230 | SYELPDGQVITIGN |
| | | | | | | ER |
| 1954.0645 | 1954.0369 | −.0276 | −14 | 96 | 113 | VAPEEHPVLLTEA |
| | | | | | | PLNPK |
| 2231.0649 | 2231.0745 | .0096 | 4 | 268 | 288 | DLYANTVLSGGTT |
| | | | | | | MYPGI |
| | | | | | | ADR |

| | |
|---|---|
| Chain J, Model Of Alpha-Actinin Ch1 Bound | gi\|295321918 |
| To F-Actin | |

Protein Group

| | |
|---|---|
| Chain A, Model Of Actin-Fimbrin Abd2 Complex | gi\|168177284 |
| Chain A, Model Of Actin-Fimbrin Abd2 Complex | |
| Chain A, Model Of Alpha-Actinin Ch1 Bound To F- | gi\|295321909 |
| Actin | |
| Chain B, Model Of Alpha-Actinin Ch1 Bound To F- | gi\|295321910 |
| Actin | |
| Chain C, Model Of Alpha-Actinin Ch1 Bound To F- | gi\|295321911 |
| Actin | |
| Chain D, Model Of Alpha-Actinin Ch1 Bound To F- | gi\|295321912 |
| Actin | |
| Chain E, Model Of Alpha-Actinin Ch1 Bound To F- | gi\|295321913 |
| Actin | |
| Chain F, Model Of Alpha-Actinin Ch1 Bound To F- | gi\|295321914 |
| Actin | |
| Chain G, Model Of Alpha-Actinin Ch1 Bound To F- | gi\|295321915 |
| Actin | |
| Chain H, Model Of Alpha-Actinin Ch1 Bound To F- | gi\|295321916 |
| Actin | |
| Chain I, Model Of Alpha-Actinin Ch1 Bound To F-Actin | gi\|295321917 |
| Unknown (protein for IMAGE: 3538275) [Homo | gi\|16924319 |
| sapiens] | |
| actin, beta [Homo sapiens] | gi\|14250401 |
| actin, beta [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.5703 | .0031 | 3 | 328 | 335 | IIAPPERK |
| 976.4483 | 976.4528 | .0045 | 5 | 18 | 27 | AGFAGDDAPR |
| 976.4483 | 976.4528 | .0045 | 5 | 18 | 27 | AGFAGDDAPR |
| 1132.527 | 1132.526 | −.001 | −1 | 196 | 205 | GYSFTTTAER |
| 1132.527 | 1132.526 | −.001 | −1 | 196 | 205 | GYSFTTTAER |
| 1198.7056 | 1198.7018 | −.0038 | −3 | 28 | 38 | AVFPSIVGRPR |
| 1198.7056 | 1198.7018 | −.0038 | −3 | 28 | 38 | AVFPSIVGRPR |
| 1354.6234 | 1354.6217 | −.0017 | −1 | 50 | 61 | DSYVGDEAQSKR |
| 1516.7028 | 1516.6896 | −.0132 | −9 | 359 | 371 | QEYDESGPSIVHR |
| 1516.7028 | 1516.6896 | −.0132 | −9 | 359 | 371 | QEYDESGPSIVHR |
| 1629.8231 | 1629.8079 | −.0152 | −9 | 196 | 209 | GYSFTTTAEREIVR |
| 1639.8361 | 1639.8278 | −.0083 | −5 | 177 | 190 | LDLAGRDLTDYLMK |
| 1790.892 | 1790.8685 | −.0235 | −13 | 238 | 253 | SYELPDGQVITIGN |
| | | | | | | ER |
| 1790.892 | 1790.8685 | −.0235 | −13 | 238 | 253 | SYELPDGQVITIGN |
| | | | | | | ER |
| 1954.0645 | 1954.0369 | −.0276 | −14 | 95 | 112 | VAPEEHPVLLTEA |
| | | | | | | PLNPK |
| 2231.0649 | 2231.0745 | .0096 | 4 | 291 | 311 | DLYANTVLSGGTT |
| | | | | | | MYPGI |
| | | | | | | ADR |

| | |
|---|---|
| RecName: Full = Actin, cytoplasmic 2; AltName: | gi\|54036678 |
| Full = Gamma-actin; Contains: | |
| RecName: Full = Actin, cytoplasmic | |
| 2, N-terminally processed | |

Protein Group

| | |
|---|---|
| ACTG1 protein [Homo sapiens] | gi\|16041790 |
| ACTG1 protein [Homo sapiens] | gi\|15929094 |
| ACTG1 protein [Homo sapiens] | gi\|12804935 |
| ACTG1 protein [Homo sapiens] | gi\|17511847 |
| Actin, beta [Homo sapiens] | gi\|15426536 |
| Actin, beta [Homo sapiens] | gi\|12654911 |
| Actin, beta [Homo sapiens] | gi\|12803203 |
| Actin, beta [Homo sapiens] | gi\|13279023 |
| Actin, beta [Homo sapiens] | |
| Actin, beta [Homo sapiens] | gi\|15928803 |
| Actin, gamma 1 [Homo sapiens] | gi\|31565757 |
| Actin, gamma 1 [Homo sapiens] | gi\|15082291 |
| Actin, gamma 1 [Homo sapiens] | gi\|12653055 |
| Actin, gamma 1 [Homo sapiens] | gi\|13938581 |
| Actin, gamma 1 [Homo sapiens] | gi\|15990564 |
| Actin, gamma 1 [Homo sapiens] | gi\|15012187 |
| Actin, gamma 1 [Homo sapiens] | gi\|14602652 |
| RecName: Full = Actin, cytoplasmic 1; AltName: | gi\|46397333 |
| Full = Beta-actin; Contains: RecName: | |
| Full = Actin, cytoplasmic 1, N-terminally | |
| processed | |
| actin, beta [Homo sapiens] | gi\|45934426 |
| actin, cytoplasmic 1 [Homo sapiens] | gi\|4501885 |
| actin, cytoplasmic 1 [Homo sapiens] | gi\|316659409 |
| actin, cytoplasmic 2 [Homo sapiens] | gi\|4501887 |
| actin, cytoplasmic 2 [Homo sapiens] | gi\|54696574 |
| actin, gamma 1 [Homo sapiens] | gi\|177968 |
| cytoplasmic beta actin [Homo sapiens] | gi\|178043 |
| gamma-actin [Homo sapiens] | gi\|28339 |
| gamma-actin [Homo sapiens] | |
| hCG15971, isoform CRA_a [Homo sapiens] | gi\|119607749 |
| hCG15971, isoform CRA_a [Homo sapiens] | gi\|119607747 |
| unknown [Homo sapiens] | gi\|30172702 |
| unnamed protein product [Homo sapiens] | gi\|284806909 |
| unnamed protein product [Homo sapiens] | gi\|283915537 |
| unnamed protein product [Homo sapiens] | gi\|229610526 |
| unnamed protein product [Homo sapiens] | gi\|213500113 |
| unnamed protein product [Homo sapiens] | gi\|210029289 |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.5703 | .0031 | 3 | 329 | 336 | IIAPPERK |
| 976.4483 | 976.4528 | .0045 | 5 | 19 | 28 | AGFAGDDAPR |
| 976.4483 | 976.4528 | .0045 | 5 | 19 | 28 | AGFAGDDAPR |
| 1132.527 | 1132.526 | −.001 | −1 | 197 | 206 | GYSFTTTAER |
| 1132.527 | 1132.526 | −.001 | −1 | 197 | 206 | GYSFTTTAER |
| 1198.7056 | 1198.7018 | −.0038 | −3 | 29 | 39 | AVFPSIVGRPR |
| 1198.7056 | 1198.7018 | −.0038 | −3 | 29 | 39 | AVFPSIVGRPR |
| 1354.6234 | 1354.6217 | −.0017 | −1 | 51 | 62 | DSYVGDEAQSKR |
| 1516.7028 | 1516.6896 | −.0132 | −9 | 360 | 372 | QEYDESGPSIVHR |
| 1516.7028 | 1516.6896 | −.0132 | −9 | 360 | 372 | QEYDESGPSIVHR |
| 1629.8231 | 1629.8079 | −.0152 | −9 | 197 | 210 | GYSFTTTAEREIVR |
| 1639.8361 | 1639.8278 | −.0083 | −5 | 178 | 191 | LDLAGRDLTDYLMK |
| 1790.892 | 1790.8685 | −.0235 | −13 | 239 | 254 | SYELPDGQVITIGN |
| | | | | | | ER |
| 1790.892 | 1790.8685 | −.0235 | −13 | 239 | 254 | SYELPDGQVITIGN |
| | | | | | | ER |
| 1954.0645 | 1954.0369 | −.0276 | −14 | 96 | 113 | VAPEEHPVLLT |
| | | | | | | EAPLNPK |
| 2231.0649 | 2231.0745 | .0096 | 4 | 292 | 312 | DLYANTVLSGG |
| | | | | | | TTMYPGI ADR |

POTE ankyrin domain family member E [Homo sapiens]

Protein Group

| | |
|---|---|
| | gi\|134133226 |
| Actin, beta [Homo sapiens] | gi\|16359158 |
| POTE ankyrin domain family member F [Homo sapiens] | gi\|153791352 |
| POTE-2 alpha-actin [Homo sapiens] | gi\|145309046 |
| PS1TP5-binding protein 1 [Homo sapiens] | gi\|93211215 |
| RecName: Full = POTE ankyrin domain family member | gi\|166898073 |
| E; AltName: Full = ANKRD26-like family C | |
| member 1A; AltName: Full = Prostate, ovary, | |
| testis-expressed | |
| protein | |
| on chromosome 2; | |
| Short = POTE-2 | |
| RecName: Full = POTE ankyrin domain family member | gi\|215273934 |
| F; AltName: Full = ANKRD26-like family C | |
| member 1B; AltName: Full = Chimeric POTE- | |
| actin | |
| protein | |
| beta actin variant [Homo sapiens] | gi\|62897671 |
| beta actin variant [Homo sapiens] | gi\|62897409 |
| beta actin variant [Homo sapiens] | gi\|62897625 |
| mutant beta-actin (beta′-actin) [Homo sapiens] | gi\|28336 |
| unnamed protein product [Homo sapiens] | gi\|194376310 |
| unnamed protein product [Homo sapiens] | |
| unnamed protein product [Homo sapiens] | gi\|66348792 |
| unnamed protein product [Homo sapiens] | gi\|28252 |
| unnamed protein product [Homo sapiens] | gi\|194375299 |
| unnamed protein product [Homo sapiens] | gi\|158257306 |

Peptide Information

| | | | | Start | End | |
|---|---|---|---|---|---|---|
| Calc. Mass | Obsrv. Mass | ±da | ±ppm | Seq. | Seq. | Sequence |
| 976.4483 | 976.4528 | .0045 | 5 | 719 | 728 | AGFAGDDAPR |
| 1198.7056 | 1198.7018 | −.0038 | −3 | 729 | 739 | AVFPSIVGRPR |
| 1516.7028 | 1516.6896 | −.0132 | −9 | 1060 | 1072 | QEYDESGPSIVHR |
| 1790.892 | 1790.8685 | −.0235 | −13 | 939 | 954 | SYELPDGQVITIGNER |

| | |
|---|---|
| Chain C, The Structure Of The Human Rnase H2 | gi\|312208155 |
| Complex Defines Key Interaction Interfaces | |
| Relevant | |
| To Enzyme Function And Human | |
| Disease | |

Protein Group

| | |
|---|---|
| AYP1 [Homo sapiens] | gi\|28628365 |
| AYP1 [Homo sapiens] | gi\|28628363 |
| AYP1 protein, isoform CRA_a [Homo sapiens] | gi\|119594839 |
| AYP1 protein, isoform CRA_b [Homo sapiens] | gi\|119594841 |
| AYP1 protein, isoform CRA_b [Homo sapiens] | gi\|119594840 |
| Chain F, The Structure Of The Human Rnase H2 | gi\|312208158 |
| Complex Defines Key Interaction Interfaces Relevant | |
| To Enzyme Function And Human | gi\|19421812 |
| Disease | |
| RNase H1 small subunit [Homo sapiens] | |
| RecName: Full = Ribonuclease H2 subunit C; | gi\|74730607 |
| Short = RNase H2 subunit C; AltName: | |
| Full = Aicardi-Goutieres syndrome 3 protein; | |
| Short = AGS3; AltName: Full = RNase H1 small | |
| subunit; | |
| AltName: Full = Ribonuclease HI subunit C | |
| Ribonuclease H2, subunit C [Homo sapiens] | gi\|23270834 |
| ribonuclease H2 subunit C [Homo sapiens] | gi\|38176285 |
| unnamed protein product [Homo sapiens] | gi\|194387324 |
| unnamed protein product [Homo sapiens] | gi\|10436946 |

Peptide Information

| | | | | Start | End | |
|---|---|---|---|---|---|---|
| Calc. Mass | Obsrv. Mass | ±da | ±ppm | Seq. | Seq. | Sequence |
| 1516.6809 | 1516.6896 | .0087 | 6 | 1 | 13 | MESGDEAAIERHR |

| | |
|---|---|
| baculoviral IAP repeat-containing 7 (livin), | gi\|119595708 |
| isoform | |
| CRA_a [Homo sapiens] | |

Protein Group

| | |
|---|---|
| Baculoviral IAP repeat-containing 7 [Homo sapiens] | gi\|15680241 |
| LIVIN [Homo sapiens] | gi\|37182790 |
| LIVIN [Homo sapiens] | gi\|37182788 |
| RecName: Full = Baculoviral IAP repeat-containing | gi\|21759008 |
| protein 7; AltName: Full = Kidney inhibitor of apoptosis | |
| protein; Short = KIAP; AltName: Full = Livin; AltName: | |
| Full = Melanoma inhibitor of apoptosis | |
| protein; Short = ML-IAP; AltName: | |
| Full = RING finger protein 50 | |
| baculoviral IAP repeat-containing 7 (livin) [Homo | gi\|13560105 |
| sapiens] | |
| baculoviral IAP repeat-containing 7 (livin) [Homo | gi\|13560104 |
| sapiens] | |
| baculoviral IAP repeat-containing 7 (livin), isoform | gi\|119595709 |
| CRA_b [Homo sapiens] | |
| baculoviral IAP repeat-containing 7 [Homo sapiens] | gi\|41387695 |
| baculoviral IAP repeat-containing protein 7 isoform | gi\|21536421 |
| alpha [Homo sapiens] | |
| baculoviral IAP repeat-containing protein 7 isoform beta | gi\|11545910 |
| [Homo sapiens] | |
| inhibitor of apoptosis [Homo sapiens] | gi\|13785205 |
| inhibitor of apoptosis [Homo sapiens] | gi\|13785204 |
| inhibitor of apoptosis protein KIAP [Homo sapiens] | gi\|11545503 |
| livin inhibitor-of-apotosis [Homo sapiens] | gi\|11245453 |

Peptide Information

| Calc. Mass | Obsrv. Mass | ±da | ±ppm | Start Seq. | End Seq. | Sequence |
|---|---|---|---|---|---|---|
| 1790.8206 | 1790.8685 | .0479 | 27 | 13 | 29 | GPQPSHWAA |
| | | | | | | GDGPTQE R |

| | |
|---|---|
| PREDICTED: hypothetical protein LOC100507747 | gi\|310110557 |
| [Homo sapiens] | |

Protein Group

| | |
|---|---|
| PREDICTED: hypothetical protein LOC100510580 | gi\|310129761 |
| [Homo sapiens] | |
| hCG2012269 [Homo sapiens] | gi\|119629636 |
| unnamed protein product [Homo sapiens] | gi\|21751293 |

Peptide Information

| Calc. | Obsrv. | | | Start | End | |
|---|---|---|---|---|---|---|
| Mass | Mass | +da | +ppm | Seq. | Seq. | Sequence |
| 1132.5164 | 1132.526 | .0096 | 8 | 365 | 374 | CSSSPRDAP |
| | | | | | | R |

| | |
|---|---|
| unnamed protein product | gi\|40042332 |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 1132.5454 | 1132.52 | — | — | 6 | 75 | GGSNXSSPSRR |

| | |
|---|---|
| unnamed protein product [Homo sapiens] | gi\|40979948 |

Peptide Information

| Calc. | Obsrv. | | | Start | End | |
|---|---|---|---|---|---|---|
| Mass | Mass | ±da | ±ppm | Seq. | Seq. | Sequence |
| 1132.6045 | 1132.526 | −.0785 | −69 | 71 | 79 | HRMFSLIGR |

| | |
|---|---|
| PREDICTED: hypothetical protein | gi\|310132586 |
| LOC100508234 | |
| [Homo sapiens] | |

Protein Group

| | |
|---|---|
| PREDICTED: hypothetical protein LOC100127987 | gi\|169212748 |
| [Homo sapiens] | |
| PREDICTED: hypothetical protein LOC100508234 | gi\|310113267 |
| [Homo sapiens] | |
| hCG1645807 [Homo sapiens] | gi\|119587028 |

Peptide Information

| Calc. | Obsrv. | | | Start | End | |
|---|---|---|---|---|---|---|
| Mass | Mass | ±da | ±ppm | Seq. | Seq. | Sequence |
| 976.4846 | 976.4528 | −.0318 | −33 | 160 | 168 | SLSGSSPWR |

| | |
|---|---|
| F-box and leucine-rich repeat protein | gi\|119609335 |
| 14, isoform | |
| CRA_b [Homo sapiens] | |

Protein Group

| | |
|---|---|
| F-box and leucine-rich repeat protein 14 [Homo | gi\|20381419 |
| sapiens] | |
| F-box and leucine-rich repeat protein 14, isoform | gi\|119609334 |
| CRA_a [Homo sapiens] | |
| F-box and leucine-rich repeat protein 14, isoform | gi\|119609336 |
| CRA_c [Homo sapiens] | |
| F-box/LRR-repeat protein 14 [Homo sapiens] | gi\|22748931 |
| RecName: Full = F-box/LRR-repeat protein 14; | gi\|48428083 |
| AltName: Full = F-box and leucine-rich repeat protein 14 | |

Peptide Information

| Calc. | Obsrv. | | | Start | End | |
|---|---|---|---|---|---|---|
| Mass | Mass | ±da | ±ppm | Seq. | Seq. | Sequence |
| 976.5058 | 976.4528 | −.053 | −54 | 129 | 137 | QITDSSLGR |

| | |
|---|---|
| hsp90 co-chaperone Cdc37-like 1 | gi\|66773038 |
| [Homo sapiens] | |

Protein Group

| | |
|---|---|
| CDC37 cell division cycle 37 homolog | gi\|119579186 |
| (S. cerevisiae)-like 1 [Homo sapiens] | |
| Cell division cycle 37 homolog (S. cerevisiae)-like 1 | gi\|15559546 |
| [Homo sapiens] | |
| RecName: Full = Hsp90 co-chaperone Cdc37-like 1; | gi\|182705252 |
| AltName: Full = Hsp90-associating relative of Cdc37 | |
| cell division cycle 37 homolog (S. cerevisiae)-like 1 | gi\|57208646 |
| [Homo sapiens] | |
| cell division cycle 37 homolog (S. cerevisiae)-like 1 | gi\|10696978 |
| [Homo sapiens] | |
| unnamed protein product [Homo sapiens] | gi\|7020874 |
| unnamed protein product [Homo sapiens] | gi\|40044754 |

Peptide Information

| | | | | | | |
|---|---|---|---|---|---|---|
| | Obsrv. | | | Start | End | |
| Calc. Mass | Mass | +da | +ppm | Seq. | Seq. | Sequence |

| | |
|---|---|
| collagen, type VI, alpha 2, isoform | gi\|119629722 |
| CRA_b [Homo sapiens] | |

Protein Group

| | |
|---|---|
| Collagen, type VI, | gi\|41350923 |
| alpha 2 [Homo | |
| RecName: Full = Collagen alpha-2(VI) | gi\|125987 |
| Precursor | |
| [Homo sapiens] | |
| [Homo sapiens] | |
| [Homo sapiens] | |
| collagen type VI alpha 2(VI) chain | gi\|242005 |
| sapiens] | |
| sapiens] | |
| sapiens] | |

Peptide Information

| Calc. | Obsrv. | | | Star | En | |
|---|---|---|---|---|---|---|
| Mass | Mass | ±da | ±ppm | Seq. | Seq. | Sequence |
| 1132.5746 | 1132.526 | −.0486 | — | 54 | 550 | GDFGLKGEPGR |

| | |
|---|---|
| arginine-rich | gi\|284164 |
| protein- | gi\|178991 |
| arginine-rich protein [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 1132.5933 | 1132.526 | −.0673 | −59 | 56 | 65 | MWATQGLAV |
| | | | | | | R |

| | |
|---|---|
| Nuraminidase [Homo sapiens] | gi\|8919622 |

Peptide Information

| Calc. | Obsrv. | | | Start | End | |
|---|---|---|---|---|---|---|
| Mass | Mass | ±da | ±ppm | Seq. | Seq. | Sequence |
| 976.5244 | 976.4528 | −.0716 | −73 | 130 | 138 | SSLLGACLR |

| | |
|---|---|
| coiled-coil-helix-coiled-coil-helix | gi\|119604206 |
| domain containing 3, isoform CRA_a | |
| [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 1132.5647 | 1132.526 | −.0387 | −34 | 6 | 13 | NSWLDWRR |

| | |
|---|---|
| hCG2011944 [Homo sapiens] | gi\|119629431 |

Peptide Information

| | | | | | End | |
|---|---|---|---|---|---|---|
| Calc. Mass | Obsrv. Mass | ±da | ±ppm | Start Seq. | Seq. | Sequence |
| 1132.6144 | 1132.526 | −.0884 | −78 | 35 | 44 | VCLNTSILGR |

| | |
|---|---|
| unknown [Homo | gi\|17104821 |
| sapiens] | |

Protein Group

| | |
|---|---|
| RecName: Full = Putative uncharacterized protein | gi\|74751647 |
| C9orf130; | |
| AltName: | |
| Full = Nasopharyngeal | |
| carcinoma-associated gene 12 protein | |
| hCG2003678 [Homo sapiens] | gi\|119613040 |

Peptide Information

| Calc. | Obsrv. | | | Start | End | |
|---|---|---|---|---|---|---|
| Mass | Mass | ±da | ±ppm | Seq. | Seq. | Sequence |
| 976.488 | 976.4528 | −.0352 | −36 | 61 | 69 | CSPSSLGLR |

4—AFCC—from Fraction 33

2 new found proteins which have been named KH19 (SEQ ID NO. KH19) and KH20(SEQ ID NO. KH20) and its functions are under investigation.

| | |
|---|---|
| IPI00893699 | Tax_Id = 9606 Gene_Symbol = C1D 20 kDa protein |

KH19 (SEQ ID NO. KH19) Peptide Information

| Calc. | Obsrv. | | | | | |
|---|---|---|---|---|---|---|
| Mass | Mass | ±da | ±ppm | Seq. | Seq. | Sequence |
| 811.3801 | 811.4196 | 0.0395 | 49 | 73 | 79 | TMMSVSR |
| 827.3749 | 827.4133 | 0.0384 | 46 | 73 | 79 | TMMSVSR |
| 831.4683 | 831.4334 | −0.0349 | −42 | 168 | 175 | NASKVANK |
| 835.4785 | 835.4451 | −0.0334 | −40 | 151 | 158 | GAASRF |
| | | | | | | VK |
| 857.4515 | 857.4796 | 0.0281 | 33 | 159 | 165 | NALWEPK |
| 913.4989 | 913.5715 | 0.0726 | 79 | 86 | 93 | LDPLEQ |
| | | | | | | AK |
| 913.4989 | 913.5715 | 0.0726 | 79 | 86 | 93 | LDPLEQ |
| | | | | | | AK |
| 1231.6833 | 1231.7903 | 0.107 | 87 | 156 | 165 | FVKNAL |
| | | | | | | WEPK |

| | |
|---|---|
| IPI00218730 | Tax_Id = 9606 Gene_Symbol = PDE6A Rod |
| | cGMP-specific 3′,5′-cyclic |
| | phosphodiesterase subunit alpha |

KH20 (SEQ ID NO. KH20) Peptide Information

| | | | | Start | End | |
|---|---|---|---|---|---|---|
| Calc. Mass | Obsrv. Mass | ±da | ±ppm | Seq. | Seq. | Sequence |
| 813.4141 | 813.4094 | −0.0047 | −6 | 312 | 317 | EINFYK |
| 817.4526 | 817.4706 | 0.018 | 22 | 828 | 834 | QKQQSAK |
| 817.4526 | 817.4706 | 0.018 | 22 | 828 | 834 | QKQQSAK |
| 826.424 | 826.3994 | −0.0246 | −30 | 678 | 683 | RTMFQK |
| 826.424 | 826.3994 | −0.0246 | −30 | 678 | 683 | RTMFQK |
| 859.4771 | 859.498 | 0.0209 | 24 | 207 | 213 | DEEILLK |
| 867.4471 | 867.4705 | 0.0234 | 27 | 630 | 636 | HHLEFGK |
| 891.4604 | 891.451 | −0.0094 | −11 | 820 | 826 | MKVQEEK |
| 895.4091 | 895.3915 | −0.0176 | −20 | 268 | 274 | AFLNCDR |
| 963.4426 | 963.4947 | 0.0521 | 54 | 94 | 100 | MSLFMYR |
| 1209.674 | 1209.7273 | 0.0533 | 44 | 535 | 544 | FHIPQEALVR |
| 1320.614 | 1320.6102 | −0.0038 | −3 | 373 | 383 | EPLDESGWMIK |
| 1350.7642 | 1350.7184 | −0.0458 | −34 | 630 | 640 | HHLEFGKTLLR |
| 1350.7642 | 1350.7184 | −0.0458 | −34 | 630 | 640 | HHLEFGKTLLR |
| 1852.0004 | 1852.0034 | 0.003 | | 2312 | 326 | EINFYKVIDYILH |
| | | | | | | GK |

| | |
|---|---|
| IPI00744887 | Tax_Id = 9606 Gene_Symbol = PLCH2 |
| | Isoform 3 of 1-phosphatidylinositol-4,5-bisphosphate |
| | phosphodiesterase eta-2 |

Peptide Information

| | | | | | End | |
|---|---|---|---|---|---|---|
| Calc. Mass | Obsrv. Mass | ±da | ±ppm | Start Seq. | Seq. | Sequence |
| 808.4498 | 808.402 | −0.0478 | −59 | 210 | 215 | VKQMFR |
| 809.3458 | 809.4174 | 0.0716 | 88 | 739 | 745 | DSMLGDR |
| 813.3705 | 813.4094 | 0.0389 | 48 | 237 | 242 | MMSTRR |
| 817.4526 | 817.4706 | 0.018 | 22 | 513 | 519 | VENTAKR |
| 817.4526 | 817.4706 | 0.018 | 22 | 512 | 518 | RVENTAK |
| 824.4447 | 824.4203 | −0.0244 | −30 | 210 | 215 | VKQMFR |
| 827.4846 | 827.4133 | −0.0713 | −86 | 1253 | 1260 | VSGPGVRR |
| 828.4799 | 828.4109 | −0.069 | −83 | 900 | 906 | SQKPGRR |
| 831.4683 | 831.4334 | −0.0349 | −42 | 892 | 899 | RTASAPTK |
| 856.5615 | 856.5053 | −0.0562 | −66 | 852 | 859 | VKQALGLK |
| 891.4934 | 891.451 | −0.0424 | −48 | 266 | 272 | FLQVEQK |
| 915.5006 | 915.4615 | −0.0391 | −43 | 1164 | 1171 | SKSNPNLR |
| 1350.7601 | 1350.7184 | −0.0417 | −31 | 1240 | 1252 | LSHSLGLPG |
| | | | | | | GTRR |
| 1350.7601 | 1350.7184 | −0.0417 | −31 | 1239 | 1251 | RLSHSLGLP |
| | | | | | | GGTR |
| 1556.7261 | 1556.8342 | 0.1081 | 69 | 152 | 165 | YLMAGISDE |
| | | | | | | DSLAR |

| | |
|---|---|
| IPI00968198 | Tax_Id = 9606 Gene_Symbol = PACRGL |
| | Uncharacterized protein |

Peptide Information

| | | | | Start | End | |
|---|---|---|---|---|---|---|
| Calc. Mass | Obsrv. Mass | ±da | ±ppm | Seq. | Seq. | Sequence |
| 851.4469 | 851.4418 | −0.0051 | −6 | 24 | 31 | TSSSTQLK |
| 963.4741 | 963.4947 | 0.0206 | 21 | 4 | 13 | SEGSGGTQLK |
| 1350.6682 | 1350.7184 | 0.0502 | 37 | 1 | 13 | MQKSEGSGGT |
| | | | | | | QLK |
| 1350.6682 | 1350.7184 | 0.0502 | 37 | 1 | 13 | MQKSEGSGGT |
| | | | | | | QLK |
| 2283.1768 | 2283.4019 | 0.2251 | 99 | 66 | 86 | TINPFGEQSRV |
| | | | | | | PSAFAAI |
| | | | | | | YSK |

| | |
|---|---|
| IPI00964698 | Tax_Id = 9606 Gene_Symbol = PACRGL |
| | Uncharacterized protein |

Peptide Information

| | | | | Start | End | |
|---|---|---|---|---|---|---|
| Calc. Mass | Obsrv. Mass | ±da | ±ppm | Seq. | Seq. | Sequence |
| 851.4469 | 851.4418 | −0.0051 | −6 | 24 | 31 | TSSSTQLK |
| 963.4741 | 963.4947 | 0.0206 | 21 | 4 | 13 | SEGSGGTQLK |
| 1350.6682 | 1350.7184 | 0.0502 | 37 | 1 | 13 | MQKSEGSGGT |
| | | | | | | QLK |
| 1350.6682 | 1350.7184 | 0.0502 | 37 | 1 | 13 | MQKSEGSGGT |
| | | | | | | QLK |
| 2283.1768 | 2283.4019 | 0.2251 | 99 | 66 | 86 | TINPFGEQSRV |
| | | | | | | PSAFAAI YSK |

| | |
|---|---|
| IPI00964149 | Tax_Id = 9606 Gene_Symbol = PACRGL |
| | Uncharacterized protein |

Peptide Information

| | | | | Start | End | |
|---|---|---|---|---|---|---|
| Calc. Mass | Obsrv. Mass | ±da | ±ppm | Seq. | Seq. | Sequence |
| 851.4469 | 851.4418 | −0.0051 | −6 | 24 | 31 | TSSSTQLK |
| 963.4741 | 963.4947 | 0.0206 | 21 | 4 | 13 | SEGSGGTQLK |
| 1350.6682 | 1350.7184 | 0.0502 | 37 | 1 | 13 | MQKSEGSGGT |
| | | | | | | QLK |
| 1350.6682 | 1350.7184 | 0.0502 | 37 | 1 | 13 | MQKSEGSGGT |
| | | | | | | QLK |
| 1657.7817 | 1657.8608 | 0.0791 | 48 | 66 | 79 | TINPVHSDDEV |
| | | | | | | FER |

| | |
|---|---|
| IPI00654552 | Tax_Id = 9606 Gene_Symbol = PLCH2 |
| | Isoform 1 of 1-phosphatidylinositol-4,5-bisphosphate |
| | phosphodiesterase eta-2 |

Peptide Information

| | | | | Start | End | |
|---|---|---|---|---|---|---|
| Calc. Mass | Obsrv. Mass | ±da | ±ppm | Seq. | Seq. | Sequence |
| 808.4498 | 808.402 | −0.0478 | −59 | 210 | 215 | VKQMFR |
| 809.3458 | 809.4174 | 0.0716 | 88 | 775 | 781 | DSMLGDR |
| 813.3705 | 813.4094 | 0.0389 | 48 | 237 | 242 | MMSTRR |
| 817.4526 | 817.4706 | 0.018 | 22 | 513 | 519 | VENTAKR |
| 817.4526 | 817.4706 | 0.018 | 22 | 512 | 518 | RVENTAK |
| 824.4447 | 824.4203 | −0.0244 | −30 | 210 | 215 | VKQMFR |
| 827.4846 | 827.4133 | −0.0713 | −86 | 1289 | 1296 | VSGPGVRR |
| 828.4799 | 828.4109 | −0.069 | −83 | 936 | 942 | SQKPGRR |
| 831.4683 | 831.4334 | −0.0349 | −42 | 928 | 935 | RTASAPTK |
| 856.5615 | 856.5053 | −0.0562 | −66 | 888 | 895 | VKQALGLK |
| 891.4934 | 891.451 | −0.0424 | −48 | 266 | 272 | FLQVEQK |
| 915.5006 | 915.4615 | −0.0391 | −43 | 1200 | 1207 | SKSNPNLR |
| 1350.7601 | 1350.7184 | −0.0417 | −31 | 1276 | 1288 | LSHSLGLPGGT |
| | | | | | | RR |
| 1350.7601 | 1350.7184 | −0.0417 | −31 | 1275 | 1287 | RLSHSLGLPGG |
| | | | | | | TR |
| 1556.7261 | 1556.8342 | 0.1081 | 69 | 152 | 165 | YLMAGISDEDS |
| | | | | | | LAR |

| | |
|---|---|
| IPI00478966 | Tax_Id = 9606 |
| | Gene_Symbol = PFKFB3 |
| | Uncharacterized protein |

Peptide Information

| | | | | Start | End | |
|---|---|---|---|---|---|---|
| Calc. Mass | Obsrv. Mass | ±da | ±ppm | Seq. | Seq. | Sequence |
| 825.4287 | 825.4099 | −0.0188 | −23 | 9 | 14 | TIYLCR |
| 837.4061 | 837.4302 | 0.0241 | 29 | 180 | 187 | SSADSSRK |
| 851.4985 | 851.4418 | −0.0567 | −67 | 38 | 45 | KFASALSK |
| 861.4828 | 861.4011 | −0.0817 | −95 | 57 | 63 | VWTSQLK |
| 963.4934 | 963.4947 | 0.0013 | 1 | 74 | 80 | LPYEQWK |
| 1245.6951 | 1245.7703 | 0.0752 | 60 | 54 | 63 | DLRVWTSQLK |

| | |
|---|---|
| IPI00247814 | Tax_Id = 9606 Gene_Symbol = NOTO |
| | Homeobox protein notochord |

Peptide Information

| Calc. | Obsrv. | | | Start | End | |
|---|---|---|---|---|---|---|
| Mass | Mass | ±da | ±ppm | Seq. | Seq. | Sequence |
| 822.4468 | 822.3993 | −0.0475 | −58 | 211 | 216 | YQKQQK |
| 827.4482 | 827.4133 | −0.0349 | −42 | 24 | 31 | SGRSPAPR |
| 849.4366 | 849.4159 | −0.0207 | −24 | 202 | 207 | VWFQNR |
| 856.4457 | 856.5053 | 0.0596 | 70 | 1 | 7 | MPSPRPR |
| 859.4632 | 859.498 | 0.0348 | 40 | 195 | 201 | LTENQVR |
| 870.5519 | 870.5197 | −0.0322 | −37 | 187 | 194 | AQLAARLK |
| 870.5519 | 870.5197 | −0.0322 | −37 | 187 | 194 | AQLAARLK |

| | |
|---|---|
| IPI00921826 | Tax_Id = 9606 |
| | Gene_Symbol = CHCHD |
| | 3 cDNA FLJ53726, |
| | highly similar to |
| | Coiled-coil-helix-coiled-coil- |
| | helixdomain-containing |
| | protein 3 |

Peptide Information

| Calc. | Obsrv. | | | Start | End | |
|---|---|---|---|---|---|---|
| Mass | Mass | ±da | ±ppm | Seq. | Seq. | Sequence |
| 817.4162 | 817.4706 | 0.0544 | 67 | 90 | 95 | ELDRER |
| 817.4162 | 817.4706 | 0.0544 | 67 | 90 | 95 | ELDRER |
| 826.3724 | 826.3994 | 0.027 | 33 | 1 | 8 | MGGTTSTR |
| 826.3724 | 826.3994 | 0.027 | 33 | 1 | 8 | MGGTTSTR |
| 835.438 | 835.4451 | 0.0071 | 8 | 2 | 9 | GGTTSTRR |
| 848.4108 | 848.3859 | −0.0249 | −29 | 113 | 119 | SEEERAK |
| 859.4631 | 859.498 | 0.0349 | 41 | 87 | 93 | QAKELDR |

| | |
|---|---|
| IPI00893693 | Tax_Id = 9606 Gene_Symbol = |
| | CCDC88A 137 kDa protein |

Peptide Information

| | | | | Start | End | |
|---|---|---|---|---|---|---|
| Calc. Mass | Obsrv. Mass | ±da | ±ppm | Seq. | Seq. | Sequence |
| 879.4968 | 879.4153 | −0.0815 | −93 | 635 | 642 | KSSMVALK |
| 880.5138 | 880.4396 | −0.0742 | −84 | 129 | 135 | YKLLESK |
| 896.4584 | 896.4399 | −0.0185 | −21 | 365 | 371 | NLEVEHR |
| 985.5789 | 985.582 | 0.0031 | 3 | 16 | 23 | LRQQAEIK |
| 985.5789 | 985.582 | 0.0031 | 3 | 16 | 23 | LRQQAEIK |
| 1021.5272 | 1021.5333 | 0.0061 | 6 | 961 | 969 | ESSLSRQSK |
| 1021.5425 | 1021.5333 | −0.0092 | −9 | 178 | 185 | NYEALKQR |
| 1187.6267 | 1187.6656 | 0.0389 | 33 | 383 | 392 | QKGQLEDLEK |
| | 1187.6656 | | | | | |
| 1187.6267 | 1187.6656 | 0.0389 | 33 | 383 | 392 | QKGQLEDLEK |
| 1199.5903 | 1199.6674 | 0.0771 | 64 | 435 | 444 | ETEVLQTDHK |
| 1254.6212 | 1254.6615 | 0.0403 | 32 | 345 | 355 | QASEYESLISK |
| 1406.7274 | 1406.6833 | −0.0441 | −31 | 372 | 382 | DLEDRYNQLLK |
| 1406.7274 | 1406.6833 | −0.0441 | −31 | 372 | 382 | DLEDRYNQLLK |
| 1428.6754 | 1428.7153 | 0.0399 | 28 | 909 | 921 | SVSGKTPGDFY |
| | | | | | | DR |
| 1479.6996 | 1479.7794 | 0.0798 | 54 | 875 | 887 | SSSQENLLDEV |
| | | | | | | MK |
| 1502.8425 | 1502.8582 | 0.0157 | 10 | 78 | 90 | TLVTLREDLVSEK |
| 1727.9286 | 1727.8947 | −0.0339 | −20 | 223 | 237 | LIEVERNNATLQ |
| | | | | | | AEK |
| 2213.1084 | 2213.2441 | 0.1357 | 61 | 935 | 955 | KTEDTYFISSAG |
| | | | | | | KPTPGT |
| | | | | | | QGK |
| 2233.0918 | 2233.0076 | −0.0842 | −38 | 515 | 532 | TLLEQNMESKD |
| | | | | | | LFHVEQR |
| 2233.0918 | 2233.2017 | 0.1099 | 49 | 515 | 532 | TLLEQNMESKD |
| | | | | | | LFHVEQR |

| | |
|---|---|
| IPI01012199 | Tax_Id = 9606 Gene_Symbol = MACF1 |
| | Uncharacterized protein |

Protein Group

| | |
|---|---|
| IPI00256861 | Tax_Id = 9606 Gene_Symbol = MACF1 Isoform 2 of |
| | Microtubule-actin cross-linking factor 1, isoforms 1/2/3/5 |

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3736 | 3743 | LMALGPIR |
| 870.5229 | 870.5385 | 0.0156 | 18 | 3736 | 3743 | LMALGPIR |
| 879.4935 | 879.4153 | −0.0782 | −89 | 1874 | 1881 | FVTISGQK |
| 880.441 | 880.4396 | −0.0014 | −2 | 2240 | 2246 | DFTELQK |
| 910.4265 | 910.4448 | 0.0183 | 20 | 3476 | 3482 | YSEIQDR |
| 910.4265 | 910.4448 | 0.0183 | 20 | 3476 | 3482 | YSEIQDR |
| 928.4669 | 928.4629 | −0.004 | −4 | 3652 | 3658 | KEVMEHR |
| 1021.5499 | 1021.5333 | −0.0166 | −16 | 2551 | 2558 | QQVQFMLK |
| 1021.5499 | 1021.5333 | −0.0166 | −16 | 2551 | 2558 | QQVQFMLK |
| 1106.5034 | 1106.5583 | 0.0549 | 50 | 1018 | 1026 | LEEEVEACK |
| 1170.6841 | 1170.6443 | −0.0398 | −34 | 3312 | 3321 | VVKAQIQEQK |
| 1187.6201 | 1187.6656 | 0.0455 | 38 | 2757 | 2766 | NCPISAKLER |
| 1187.6201 | 1187.6656 | 0.0455 | 38 | 2757 | 2766 | NCPISAKLER |
| 1199.6896 | 1199.6674 | −0.0222 | −19 | 3758 | 3767 | AFSIDIIRHK |
| 1257.6797 | 1257.6525 | −0.0272 | −22 | 1506 | 1516 | QISEQLNALNK |
| 1257.6797 | 1257.6525 | −0.0272 | −22 | 1506 | 1516 | QISEQLNALNK |
| 1261.694 | 1261.6499 | −0.0441 | −35 | 380 | 389 | LLEVWIEFGR |
| 1287.6791 | 1287.6593 | −0.0198 | −15 | 4662 | 4672 | EKTLLPEDSQK |
| 1320.7271 | 1320.6016 | −0.1255 | −95 | 1870 | 1881 | GDLRFVTISGQK |
| 1406.7386 | 1406.6833 | −0.0553 | −39 | 4647 | 4658 | QPVYDTTIRTGR |
| 1406.7386 | 1406.6833 | −0.0553 | −39 | 4647 | 4658 | QPVYDTTIRTGR |
| 1413.7809 | 1413.8057 | 0.0248 | 18 | 3156 | 3167 | ARQEQLELTLGR |
| 1420.7213 | 1420.6881 | −0.0332 | −23 | 2940 | 2951 | TGSLEEMTQRLR |
| 1425.7156 | 1425.8075 | 0.0919 | 64 | 869 | 880 | NTISVKAVCDYR |
| 1428.7693 | 1428.7153 | −0.054 | −38 | 5052 | 5063 | LNDALDRLEELK |
| 1465.7281 | 1465.7726 | 0.0445 | 30 | 4428 | 4439 | EETYNQLLDKGR |
| 1465.7316 | 1465.7726 | 0.041 | 28 | 4440 | 4453 | LMLLSRDDSGS |
| | | | | | | GSK |
| 1487.7952 | 1487.7654 | −0.0298 | −20 | 3565 | 3577 | QTTGEEVLLIQ |
| | | | | | | EK |
| 1502.873 | 1502.8582 | −0.0148 | −10 | 380 | 391 | LLEVWIEFGRIK |
| 1532.6785 | 1532.7728 | 0.0943 | 62 | 3891 | 3903 | ELNPEEGEMVE |
| | | | | | | EK |
| 1713.8728 | 1713.8539 | −0.0189 | −11 | 3123 | 3137 | HMLEEEGTLDL |
| | | | | | | LGLK |
| 1727.9149 | 1727.8947 | −0.0202 | −12 | 2151 | 2165 | KLLPQAEMFEH |
| | | | | | | LSGK |
| 1794.9636 | 1794.8103 | −0.1533 | −85 | 5106 | 5121 | QEFIDGILASKF |
| | | | | | | PTTK |
| 1838.8412 | 1838.927 | 0.0858 | 47 | 4960 | 4974 | ALIAEHQTFME |
| | | | | | | EMTR |
| 2186.155 | 2185.9851 | −0.1699 | −78 | 1958 | 1978 | LLSDTVASDPG |
| | | | | | | VLQEQLA |
| | | | | | | TTK |
| 2202.1799 | 2201.9719 | −0.208 | −94 | 2864 | 2882 | MSELRVTLDPV |
| | | | | | | QLESSLLR |
| | | | | | | EALAGLLVTYP |
| 2233.1135 | 2233.0076 | −0.1059 | −47 | 2462 | 2481 | NSQEAEN |
| | | | | | | WK |
| 2233.1135 | 2233.2017 | 0.0882 | 39 | 2462 | 2481 | EALAGLLVTYP |
| | | | | | | NSQEAEN |
| | | | | | | WK |
| 2299.0217 | 2299.144 | 0.1223 | 53 | 3068 | 3088 | EMFSQLADLDD |
| | | | | | | ELDGMG |
| | | | | | | AIGR |

| | |
|---|---|
| IPI00982053 | Tax_Id = 9606 |
| | Gene_Symbol = TSSK6 Conserved |
| | hypothetical |
| | protein |

Peptide Information

| | | | | Start | End | |
|---|---|---|---|---|---|---|
| Calc. Mass | Obsrv. Mass | ±da | ±ppm | Seq. | Seq. | Sequence |
| 856.4999 | 856.5223 | 0.0224 | 26 | 89 | 97 | AAQIAGAVR |
| 879.4907 | 879.4153 | −0.0754 | −86 | 55 | 62 | ATPAHRAR |
| 880.3876 | 880.4396 | 0.052 | 59 | 267 | 273 | GNMRSCR |
| 896.3825 | 896.4399 | 0.0574 | 64 | 267 | 273 | GNMRSCR |
| 912.4574 | 912.4597 | 0.0023 | 3 | 125 | 132 | LTDFGFGR |
| 1187.6136 | 1187.6656 | 0.052 | 44 | 271 | 279 | SCRVLLHMR |
| 1187.6136 | 1187.6656 | 0.052 | 44 | 271 | 279 | SCRVLLHMR |
| 1332.6768 | 1332.6146 | −0.0622 | −47 | 26 | 40 | GHQGGGPAAS |
| | | | | | | APGLR |
| 1413.771 | 1413.8057 | 0.0347 | 25 | 148 | 160 | GAPGHPLRPQ |
| | | | | | | EVR |
| 1487.7272 | 1487.7654 | 0.0382 | 26 | 111 | 122 | CENVLLSPDERR |
| 2299.2095 | 2299.144 | −0.0655 | −28 | 240 | 260 | LEAGWFQPFL |
| | | | | | | QPRALGQ |
| | | | | | | GGAR |

| | |
|---|---|
| IPI01018834 | Tax_Id = 9606 Gene_Symbol = MACF1 |
| | Uncharacterized protein |

Protein Group

| | |
|---|---|
| IPI00478226 | Tax_Id = 9606 Gene_Symbol = MACF1 Isoform 5 of |
| | Microtubule-actin cross-linking factor 1, isoforms 1/2/3/5 |

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3606 | 3613 | LMALGPIR |
| 870.5229 | 870.5385 | 0.0156 | 18 | 3606 | 3613 | LMALGPIR |
| 879.4935 | 879.4153 | −0.0782 | −89 | 1874 | 1881 | FVTISGQK |
| 880.441 | 880.4396 | −0.0014 | −2 | 2240 | 2246 | DFTELQK |
| 928.4669 | 928.4629 | −0.004 | −4 | 3522 | 3528 | KEVMEHR |
| 1021.5499 | 1021.5333 | −0.0166 | −16 | 2530 | 2537 | QQVQFMLK |
| 1021.5499 | 1021.5333 | −0.0166 | −16 | 2530 | 2537 | QQVQFMLK |
| 1106.5034 | 1106.5583 | 0.0549 | 50 | 1018 | 1026 | LEEEVEACK |
| 1170.6841 | 1170.6443 | −0.0398 | −34 | 3291 | 3300 | VVKAQIQEQK |
| 1187.6201 | 1187.6656 | 0.0455 | 38 | 2736 | 2745 | NCPISAKLER |
| 1187.6201 | 1187.6656 | 0.0455 | 38 | 2736 | 2745 | NCPISAKLER |
| 1199.6896 | 1199.6674 | −0.0222 | −19 | 3628 | 3637 | AFSIDIIRHK |
| 1257.6797 | 1257.6525 | −0.0272 | −22 | 1506 | 1516 | QISEQLNA |
| | | | | | | LNK |
| 1257.6797 | 1257.6525 | −0.0272 | −22 | 1506 | 1516 | QISEQLNA |
| | | | | | | LNK |
| 1261.694 | 1261.6499 | −0.0441 | −35 | 380 | 389 | LLEVWIEF |
| | | | | | | GR |
| 1287.6791 | 1287.6593 | −0.0198 | −15 | 4532 | 4542 | EKTLLPED |
| | | | | | | SQK |
| 1320.7271 | 1320.6016 | −0.1255 | −95 | 1870 | 1881 | GDLRFVTI |
| | | | | | | SGQK |
| 1406.7386 | 1406.6833 | −0.0553 | −39 | 4517 | 4528 | QPVYDTTI |
| | | | | | | RTGR |
| 1406.7386 | 1406.6833 | −0.0553 | −39 | 4517 | 4528 | QPVYDTTI |
| | | | | | | RTGR |
| 1413.7809 | 1413.8057 | 0.0248 | 18 | 3135 | 3146 | ARQEQLE |
| | | | | | | LTLGR |
| 1420.7213 | 1420.6881 | −0.0332 | −23 | 2919 | 2930 | TGSLEEMTQRLR |
| 1425.7156 | 1425.8075 | 0.0919 | 64 | 869 | 880 | NTISVKAVCDYR |
| 1428.7693 | 1428.7153 | −0.054 | −38 | 4922 | 4933 | LNDALDRLEELK |
| 1465.7281 | 1465.7726 | 0.0445 | 30 | 4298 | 4309 | EETYNQLLDKGR |
| 1465.7316 | 1465.7726 | 0.041 | 28 | 4310 | 4323 | LMLLSRDDSGS |
| | | | | | | GSK |
| 1487.7952 | 1487.7654 | −0.0298 | −20 | 3435 | 3447 | QTTGEEVLLIQ |
| | | | | | | EK |
| 1502.873 | 1502.8582 | −0.0148 | −10 | 380 | 391 | LLEVWIEFGRIK |
| 1532.6785 | 1532.7728 | 0.0943 | 62 | 3761 | 3773 | ELNPEEGEMVE |
| | | | | | | EK |
| 1713.8728 | 1713.8539 | −0.0189 | −11 | 3102 | 3116 | HMLEEEGTLDL |
| | | | | | | LGLK |
| 1727.9149 | 1727.8947 | −0.0202 | −12 | 2151 | 2165 | KLLPQAEMFEH |
| | | | | | | LSGK |
| 1794.9636 | 1794.8103 | −0.1533 | −85 | 4976 | 4991 | QEFIDGILASKF |
| | | | | | | PTTK |
| 1838.8412 | 1838.927 | 0.0858 | 47 | 4830 | 4844 | ALIAEHQTFME |
| | | | | | | EMTR |
| 2186.155 | 2185.9851 | −0.1699 | −78 | 1958 | 1978 | LLSDTVASDPG |
| | | | | | | VLQEQLA |
| | | | | | | TTK |
| 2202.1799 | 2201.9719 | −0.208 | −94 | 2843 | 2861 | MSELRVTLDPV |
| | | | | | | QLESSLLR |
| 2233.1135 | 2233.0076 | −0.1059 | −47 | 2441 | 2460 | EALAGLLVTYP |
| | | | | | | NSQEAEN |
| | | | | | | WK |
| 2233.1135 | 2233.2017 | 0.0882 | 39 | 2441 | 2460 | EALAGLLVTYP |
| | | | | | | NSQEAEN |
| | | | | | | WK |
| 2299.0217 | 2299.144 | 0.1223 | 53 | 3047 | 3067 | EMFSQLADLDD |
| | | | | | | ELDGMG |
| | | | | | | AIGR |

| | |
|---|---|
| IPI01018940 | Tax_Id = 9606 Gene_Symbol = MACF1 Isoform 3 of |
| | Microtubule-actin cross-linking factor 1, |
| | isoforms 1/2/3/5 |

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3680 | 3687 | LMALGPIR |
| 870.5229 | 870.5385 | 0.0156 | 18 | 3680 | 3687 | LMALGPIR |
| 879.4935 | 879.4153 | −0.0782 | −89 | 1839 | 1846 | FVTISGQK |
| 880.441 | 880.4396 | −0.0014 | −2 | 2205 | 2211 | DFTELQK |
| 910.4265 | 910.4448 | 0.0183 | 20 | 3420 | 3426 | YSEIQDR |
| 910.4265 | 910.4448 | 0.0183 | 20 | 3420 | 3426 | YSEIQDR |
| 928.4669 | 928.4629 | −0.004 | −4 | 3596 | 3602 | KEVMEHR |
| 1021.5499 | 1021.5333 | −0.0166 | −16 | 2495 | 2502 | QQVQFMLK |
| 1021.5499 | 1021.5333 | −0.0166 | −16 | 2495 | 2502 | QQVQFM |
| | | | | | | LK |
| 1106.5034 | 1106.5583 | 0.0549 | 50 | 983 | 991 | LEEEVE |
| | | | | | | ACK |
| 1170.6841 | 1170.6443 | −0.0398 | −34 | 3256 | 3265 | VVKAQI |
| | | | | | | QEQK |
| 1187.6201 | 1187.6656 | 0.0455 | 38 | 2701 | 2710 | NCPISAK |
| | | | | | | LER |
| 1187.6201 | 1187.6656 | 0.0455 | 38 | 2701 | 2710 | NCPISAKLER |
| 1199.6896 | 1199.6674 | −0.0222 | −19 | 3702 | 3711 | AFSIDIIRHK |
| 1257.6797 | 1257.6525 | −0.0272 | −22 | 1471 | 1481 | QISEQLNALNK |
| 1257.6797 | 1257.6525 | −0.0272 | −22 | 1471 | 1481 | QISEQLNALNK |
| 1261.694 | 1261.6499 | −0.0441 | −35 | 345 | 354 | LLEVWIEFGR |
| 1287.6791 | 1287.6593 | −0.0198 | −15 | 4606 | 4616 | EKTLLPEDSQK |
| 1320.7271 | 1320.6016 | −0.1255 | −95 | 1835 | 1846 | GDLRFVTISGQK |
| 1406.7386 | 1406.6833 | −0.0553 | −39 | 4591 | 4602 | QPVYDTTIRTGR |
| 1406.7386 | 1406.6833 | −0.0553 | −39 | 4591 | 4602 | QPVYDTTIRTGR |
| 1413.7809 | 1413.8057 | 0.0248 | 18 | 3100 | 3111 | ARQEQLELTLGR |
| 1420.7213 | 1420.6881 | −0.0332 | −23 | 2884 | 2895 | TGSLEEMTQRLR |
| 1425.7156 | 1425.8075 | 0.0919 | 64 | 834 | 845 | NTISVKAVCDYR |
| 1428.7693 | 1428.7153 | −0.054 | −38 | 4996 | 5007 | LNDALDRLEELK |
| 1465.7281 | 1465.7726 | 0.0445 | 30 | 4372 | 4383 | EETYNQLLDKGR |
| 1465.7316 | 1465.7726 | 0.041 | 28 | 4384 | 4397 | LMLLSRDDSGSGSK |
| 1487.7952 | 1487.7654 | −0.0298 | −20 | 3509 | 3521 | QTTGEEVLLIQ |
| | | | | | | EK |
| 1502.873 | 1502.8582 | −0.0148 | −10 | 345 | 356 | LLEVWIEFGRIK |
| 1532.6785 | 1532.7728 | 0.0943 | 62 | 3835 | 3847 | ELNPEEGEMVE |
| | | | | | | EK |
| 1713.8728 | 1713.8539 | −0.0189 | −11 | 3067 | 3081 | HMLEEEGTLDL |
| | | | | | | LGLK |
| 1727.9149 | 1727.8947 | −0.0202 | −12 | 2116 | 2130 | KLLPQAEMFEH |
| | | | | | | LSGK |
| 1794.9636 | 1794.8103 | −0.1533 | −85 | 5050 | 5065 | QEFIDGILASKF |
| | | | | | | PTTK |
| 1838.8412 | 1838.927 | 0.0858 | 47 | 4904 | 4918 | ALIAEHQTFME |
| | | | | | | EMTR |
| 2186.155 | 2185.9851 | −0.1699 | −78 | 1923 | 1943 | LLSDTVASDPG |
| | | | | | | VLQEQLA |
| | | | | | | TTK |
| 2202.1799 | 2201.9719 | −0.208 | −94 | 2808 | 2826 | MSELRVTLDPV |
| | | | | | | QLESSLLR |
| 2233.1135 | 2233.0076 | −0.1059 | −47 | 2406 | 2425 | EALAGLLVTYP |
| | | | | | | NSQEAEN |
| | | | | | | WK |
| 2233.1135 | 2233.2017 | 0.0882 | 39 | 2406 | 2425 | EALAGLLVTYP |
| | | | | | | NSQEAEN |
| | | | | | | WK |
| 2299.0217 | 2299.144 | 0.1223 | 53 | 3012 | 3032 | EMFSQLADLDD |
| | | | | | | ELDGMG |
| | | | | | | AIGR |

| | |
|---|---|
| IPI00152653 | Tax_Id = 9606 |
| | Gene_Symbol = DNAH5 Dynein heavy |
| | chain 5, axonemal |

Peptide Information

| | | | | Start | End | |
|---|---|---|---|---|---|---|
| Calc. Mass | Obsrv. Mass | ±da | ±ppm | Seq. | Seq. | Sequence |
| 856.525 | 856.5223 | −0.0027 | −3 | 1408 | 1414 | QLNLLQK |
| 879.4683 | 879.4153 | −0.053 | −60 | 1654 | 1660 | RFSNIDK |
| 880.4774 | 880.4396 | −0.0378 | −43 | 1204 | 1211 | FALTAETK |
| 896.4407 | 896.4399 | −0.0008 | −1 | 747 | 753 | RNFSNMK |
| 910.488 | 910.4448 | −0.0432 | −47 | 1702 | 1709 | SLTGYLEK |
| 910.488 | 910.4448 | −0.0432 | −47 | 1702 | 1709 | SLTGYLEK |
| 912.4573 | 912.4597 | 0.0024 | 3 | 285 | 291 | AELEHWK |
| 928.5403 | 928.4629 | −0.0774 | −83 | 4440 | 4446 | IPAWWKK |
| 985.5941 | 985.582 | −0.0121 | −12 | 2503 | 2509 | RLELWLR |
| 985.5941 | 985.582 | −0.0121 | −12 | 2503 | 2509 | RLELWLR |
| 1005.5363 | 1005.6074 | 0.0711 | 71 | 820 | 827 | VNDLIEFR |
| 1021.4805 | 1021.5333 | 0.0528 | 52 | 2103 | 2111 | SVAMMVPDR |
| 1021.4805 | 1021.5333 | 0.0528 | 52 | 2103 | 2111 | SVAMMVPDR |
| 1106.5411 | 1106.5583 | 0.0172 | 16 | 326 | 333 | TWREMDIR |
| 1187.6816 | 1187.6656 | −0.016 | −13 | 4549 | 4558 | NMKLIESKPK |
| 1187.6816 | 1187.6656 | −0.016 | −13 | 4549 | 4558 | NMKLIESKPK |
| 1199.6995 | 1199.6674 | −0.0321 | −27 | 2585 | 2596 | AVLLIGEQGTAK |
| 1257.7566 | 1257.6525 | −0.1041 | −83 | 167 | 177 | LLSDIFIPALR |
| 1257.7566 | 1257.6525 | −0.1041 | −83 | 167 | 177 | LLSDIFIPALR |
| 1261.6212 | 1261.6499 | 0.0287 | 23 | 1299 | 1308 | VDTLHYAWEK |
| 1271.6553 | 1271.6659 | 0.0106 | 8 | 3711 | 3721 | TSIIDFTVTMK |
| 1332.7369 | 1332.6146 | −0.1223 | −92 | 3210 | 3222 | LKEASESVAAL |
| | | | | | | SK |
| 1413.8577 | 1413.8057 | −0.052 | −37 | 166 | 177 | RLLSDIFIPALR |
| 1428.7482 | 1428.7153 | −0.0329 | −23 | 3698 | 3710 | LPNPAYTPEISAR |
| 1502.9153 | 1502.8582 | −0.0571 | −38 | 1119 | 1132 | LVSVLSTIINSTKK |
| 1794.7972 | 1794.8103 | 0.0131 | 7 | 748 | 761 | NFSNMKMMLA |
| | | | | | | EYQR |
| 1838.8668 | 1838.927 | 0.0602 | 33 | 3501 | 3515 | ERWTEQSQEF |
| | | | | | | AAQTK |
| 2266.176 | 2266.0767 | −0.0993 | −44 | 957 | 975 | ELLSHFNHQNM |
| | | | | | | DALLKVT R |

| | |
|---|---|
| IPI00966721 | Tax_Id = 9606 Gene_Symbol = |
| | C5orf28 Uncharacterized protein |

Peptide Information

| Calc. | | | | Start | End | |
|---|---|---|---|---|---|---|
| Mass | Obsrv. Mass | ±da | ±ppm | Seq. | Seq. | Sequence |
| 870.5043 | 870.5385 | 0.0342 | 39 | 2 | 9 | EAALTLPR |
| 870.5043 | 870.5385 | 0.0342 | 39 | 2 | 9 | EAALTLPR |

| | |
|---|---|
| IPI00853050 | Tax_Id = 9606 |
| | Gene_Symbol = MB21D1 |
| | Uncharacterized protein |

Peptide Information

| Calc. Mass | Obsrv. Mass | ±da | ±ppm | Start Seq. | End Seq. | Sequence |
|---|---|---|---|---|---|---|
| 924.4897 | 924.4626 | −0.0271 | −29 | 385 | 392 | EILNNHGK |
| 985.5425 | 985.582 | 0.0395 | 40 | 157 | 166 | DAAPGASKLR |
| 985.5425 | 985.582 | 0.0395 | 40 | 157 | 166 | DAAPGASKLR |
| 1021.6153 | 1021.5333 | −0.082 | −80 | 188 | 196 | GVVDHLLLR |
| 1021.6153 | 1021.5333 | −0.082 | −80 | 188 | 196 | GVVDHLLLR |
| 1254.6161 | 1254.6615 | 0.0454 | 36 | 2 | 11 | QPWHGKAM |
| | | | | | | QR |
| 1257.5422 | 1257.6525 | 0.1103 | 88 | 496 | 505 | NNEFPVFDEF |
| 1257.5422 | 1257.6525 | 0.1103 | 88 | 496 | 505 | NNEFPVFDEF |
| 1271.7206 | 1271.6659 | −0.0547 | −43 | 303 | 315 | GGSPAVTLL |
| | | | | | | ISEK |
| 1287.6652 | 1287.6593 | −0.0059 | −5 | 12 | 25 | ASEAGATAP |
| | | | | | | KASAR |
| 1413.6719 | 1413.8057 | 0.1338 | 95 | 220 | 231 | ISAPNEFDV |
| | | | | | | MFK |
| 1479.7472 | 1479.7794 | 0.0322 | 22 | 174 | 187 | LSRDDISTA |
| | | | | | | AGMVK |

| | |
|---|---|
| IPI00854821 | Tax_Id = 9606 Gene_Symbol = DLG5 Isoform 4 of Disks |
| | large homolog 5 |

Peptide Information

| | | | | Start | End | |
|---|---|---|---|---|---|---|
| Calc. Mass | Obsrv. Mass | ±da | ±ppm | Seq. | Seq. | Sequence |
| 879.3876 | 879.4153 | 0.0277 | 31 | 132 | 138 | DDVDMLR |
| 880.4523 | 880.4396 | −0.0127 | −14 | 206 | 212 | DYDALRK |
| 924.4421 | 924.4626 | 0.0205 | 22 | 1766 | 1772 | LEQEYSR |
| 985.5537 | 985.582 | 0.0283 | 29 | 139 | 146 | RENGQLLR |
| 985.5537 | 985.582 | 0.0283 | 29 | 139 | 146 | RENGQLLR |
| 1187.5917 | 1187.6656 | 0.0739 | 62 | 712 | 722 | AHGPEVQAHNK |
| 1187.5917 | 1187.6656 | 0.0739 | 62 | 712 | 722 | AHGPEVQAHNK |
| 1261.6205 | 1261.6499 | 0.0294 | 23 | 358 | 368 | KAANEEMEALR |
| 1406.7526 | 1406.6833 | −0.0693 | −49 | 1495 | 1506 | LADVEQELSFKK |
| 1406.7526 | 1406.6833 | −0.0693 | −49 | 1495 | 1506 | LADVEQELSFKK |
| 1420.7026 | 1420.6881 | −0.0145 | −10 | 1562 | 1575 | DDNSATKTLSA |
| | | | | | | AAR |
| 1487.8315 | 1487.7654 | −0.0661 | −44 | 339 | 351 | LQTEVELAESKLK |
| 1502.7632 | 1502.8582 | 0.095 | 63 | 359 | 371 | AANEEMEALRQIK |
| 1727.9539 | 1727.8947 | −0.0592 | −34 | 1243 | 1259 | VQKGSEPLGISI |
| | | | | | | VSGEK |

| | |
|---|---|
| IPI00927275 | Tax_Id = 9606 Gene_Symbol = LMCD1 Uncharacterized |
| | protein |

Peptide Information

| Calc. Mass | Obsrv. Mass | ±da | ±ppm | Start Seq. | End Seq. | Sequence |
|---|---|---|---|---|---|---|
| 870.5043 | 870.5385 | 0.0342 | 39 | 7 | 14 | DLNPGVKK |
| 870.5043 | 870.5385 | 0.0342 | 39 | 7 | 14 | DLNPGVKK |
| 1106.5623 | 1106.5583 | −0.004 | −4 | 15 | 24 | MSLGQLQSAR |
| 1420.6063 | 1420.6881 | 0.0818 | 58 | 33 | 44 | GTCSGFEPHSWR |
| 2265.9951 | 2266.0767 | 0.0816 | 36 | 25 | 44 | GVACLGCKGTC |
| | | | | | | SGFEPH |

### 5—Fraction IV

3 new found proteins in fraction IV which are named KH21 (SEQ ID NO. KH21), KH22 (SEQ ID NO. KH22) and KH23 (SEQ ID NO. 23) and their functions are being investigated. Following is their sequence.

| | |
|---|---|
| 11/A11 | Instr./Gel Origin |
| [1] Sample Project | Instrument |
| 20110325 | Sample |
| | Name |

| | |
|---|---|
| Protein Name | Accession No. |
| Tax_Id = 9606 Gene_Symbol = | IPI00966826 |
| PDS5A Protein | |
| KH21 (SEQ ID NO. KH21) Peptide | |
| Information | |

| Calc. Mass | Obsrv. Mass | ±da | ±ppm | Start Seq. | End Seq. | Sequence |
|---|---|---|---|---|---|---|
| 837.3705 | 837.4229 | .0524 | 63 | 146 | 151 | CQNMLR |
| | 837.4229 | | | | | |
| 842.4479 | 842.4476 | −.0003 | 0 | 16 | 22 | NPNKDVR |
| 875.4985 | 875.4897 | −.0088 | −10 | 81 | 87 | VSWIKDK |
| 1193.6525 | 1193.5586 | −.0939 | −79 | 156 | 165 | ELLDLHKQPT |
| 1254.6221 | 1254.6885 | .0664 | 53 | 55 | 65 | EAMMGLAQLYK |
| 1254.6221 | 1254.6885 | .0664 | 53 | 55 | 65 | EAMMGLAQLYK |
| 1732.8905 | 1732.8311 | −.0594 | −34 | 35 | 49 | IYAPEAPYTSH |
| | | | | | | DKLK |
| 1732.8905 | 1732.8311 | −.0594 | −34 | 35 | 49 | IYAPEAPYTSH |
| | | | | | | DKLK |

| | |
|---|---|
| Tax_Id = 9606 Gene_Symbol = - Hypothetical short | IPI00796008 |
| protein | |

Peptide Information

| Calc. Mass | Obsrv. Mass | ±da | ±ppm | Start Seq. | End Seq. | Sequence |
|---|---|---|---|---|---|---|
| 831.4067 | 831.4734 | .0667 | 80 | 12 | 19 | KGENGNGR |
| 919.4995 | 919.4981 | −.0014 | −2 | 5 | 11 | NYPLEKR |
| 919.4995 | 919.4981 | −.0014 | −2 | 5 | 11 | NYPLEKR |
| 1208.6093 | 1208.5834 | −.0259 | −21 | 1 | 10 | MASRNYPLEK |
| 1208.6093 | 1208.5834 | −.0259 | −21 | 1 | 10 | MASRNYPLEK |
| 1529.858 | 1529.8091 | −.0489 | −32 | 20 | 32 | MQLLSLRQLGA |
| | | | | | | QR |

| | |
|---|---|
| Tax_Id = 9606 Gene_Symbol = PACRGL Uncharacterized | IPI00964510 |
| protein | |

Peptide Information

| Calc. Mass | Obsrv. Mass | ±da | ±ppm | Start Seq. | End Seq. | Sequence |
|---|---|---|---|---|---|---|
| 851.4469 | 851.448 | .0011 | 1 | 24 | 31 | TSSSTQLK |
| 963.4741 | 963.5344 | .0603 | 63 | 4 | 13 | SEGSGGTQLK |
| 1208.5753 | 1208.5834 | .0081 | 7 | 41 | 52 | SSLSTSSPESAR |
| 1208.5753 | 1208.5834 | .0081 | 7 | 41 | 52 | SSLSTSSP |
| 1388.6007 | 1388.6624 | .0617 | 44 | 77 | 87 | SKIPTYCSI |

| | |
|---|---|
| Tax_Id = 9606 Gene_Symbol = DUS3L Isoform 3 of | IPI00186125 |
| tRNA-dihydrouridine synthase 3-like | |

Peptide Information

| Calc. Mass | Obsrv. Mass | ±da | ±ppm | Start Seq. | End Seq. | Sequence |
|---|---|---|---|---|---|---|
| 824.4261 | 824.4028 | −.0233 | −28 | 228 | 233 | EQRYTK |
| 831.4471 | 831.4734 | .0263 | 32 | 402 | 408 | HKANAYK |
| 875.4468 | 875.4897 | .0429 | 49 | 56 | 62 | ETELDIR |
| 1292.6263 | 1292.6439 | .0176 | 14 | 53 | 62 | TCRETELDIR |
| 1364.7025 | 1364.6797 | −.0228 | −17 | 267 | 279 | AMQTGVTGIMI |
| | | | | | | AR |
| 1529.7091 | 1529.8091 | .1 | 65 | 293 | 304 | EQRHWDISSS |
| | | | | | | ER |
| 2317.0361 | 2316.875 | −.1611 | −70 | 1 | 25 | MAEGTAEAPL |
| | | | | | | ENGGGG D |
| | | | | | | SGAGALER |

| | |
|---|---|
| Tax_Id = 9606 Gene_Symbol = GRB10 | IPI00879698 |
| Uncharacterized protein | |

Peptide Information

| Calc. Mass | Obsrv. Mass | ±da | ±ppm | Start Seq. | End Seq. | Sequence |
|---|---|---|---|---|---|---|
| 963.5622 | 963.5344 | −.0278 | −29 | 106 | 114 | SQPVHILAV |
| 972.5109 | 972.439 | −.0719 | −74 | 12 | 19 | DKVEQTPR |
| 990.5189 | 990.4394 | −.0795 | −80 | 1 | 9 | MQAAGPLFR |
| 1221.6409 | 1221.6112 | −.0297 | −24 | 1 | 11 | MQAAGPLFRSK |

| | |
|---|---|
| Tax_Id = 9606 Gene_Symbol = POLR1B 10 kDa protein | IPI00916605 |

Peptide Information

| Calc. Mass | Obsrv. Mass | ±da | ±ppm | Start Seq. | End Seq. | Sequence |
|---|---|---|---|---|---|---|
| 807.3777 | 807.3706 | −.0071 | −9 | 72 | 78 | GQCLSSR |
| 824.4485 | 824.4028 | −.0457 | −55 | 66 | 71 | RDHLQR |
| 1004.473 | 1004.4658 | −.0072 | −7 | 1 | 8 | MDPGSRWR |
| 1254.6953 | 1254.6885 | −.0068 | −5 | 7 | 17 | WRNLPSGPSLK |
| 1254.6953 | 1254.6885 | −.0068 | −5 | 7 | 17 | WRNLPSGPSLK |

| | |
|---|---|
| Tax_Id = 9606 Gene_Symbol = LCP1 Conserved | IPI00921552 |
| hypothetical protein | |

Peptide Information

| Calc. Mass | Obsrv. Mass | ±da | ±ppm | Start Seq. | End Seq. | Sequence |
|---|---|---|---|---|---|---|
| 919.5359 | 919.4981 | −.0378 | −41 | 30 | 37 | ALEGRVFK |
| 919.5359 | 919.4981 | −.0378 | −41 | 30 | 37 | ALEGRVFK |
| 963.5145 | 963.5344 | .0199 | 21 | 51 | 59 | AEGFLVAEK |
| 1392.7305 | 1392.6851 | −.0454 | −33 | 61 | 72 | QNLMTFG |
| | | | | | | TPVLR |
| 1529.8607 | 1529.8091 | −.0516 | −34 | 38 | 50 | MLDLVQA |
| | | | | | | QILELK |

| | |
|---|---|
| Tax_Id = 9606 | IPI00794678 |
| Gene_Symbol = LOC441528; HLA-A; LOC729162 | |
| MHC | |
| class I antigen | |

Peptide Information

| Calc. | Obsrv. | | | Start | End | |
|---|---|---|---|---|---|---|
| Mass | Mass | ±da | ±ppm | Seq. | Seq. | Sequence |
| 805.3798 | 805.3078 | −.072 | −89 | 93 | 99 | AQSQTDR |
| 807.3851 | 807.3706 | −.0145 | −18 | 1 | 7 | MAVMAPR |
| 963.4741 | 963.5344 | .0603 | 63 | 311 | 319 | ESRSLS |
| | | | | | | GDL |
| 1146.5862 | 1146.4808 | −.1054 | −92 | 90 | 99 | NVKAQS |
| | | | | | | QTDR |
| 1146.5862 | 1146.4808 | −.1054 | −92 | 90 | 99 | NVKAQS |
| | | | | | | QTDR |
| 1221.611 | 1221.6112 | .0002 | 0 | 146 | 155 | DYIALN |
| | | | | | | EDLR |
| 1392.6537 | 1392.6851 | .0314 | 23 | 301 | 313 | EGDGGV |
| | | | | | | MSLRESR |

| | |
|---|---|
| Tax_Id = 9606 Gene_Symbol = - | IPI00925836 |
| 37 kDa protein | |

Peptide Information

| Calc. | Obsrv. | | | Start | End | |
|---|---|---|---|---|---|---|
| Mass | Mass | ±da | ±ppm | Seq. | Seq. | Sequence |
| 805.3807 | 805.3078 | −.0729 | −91 | 39 | 45 | RPCGMGK |
| 1221.6344 | 1221.6112 | −.0232 | −19 | 39 | 48 | RPCGMG |
| | | | | | | KFLR |
| 1404.6941 | 1404.6514 | −.0427 | −30 | 73 | 84 | SCVWDG |
| | | | | | | NTPLKK |
| 1732.8785 | 1732.8311 | −.0474 | −27 | 114 | 129 | SVGPETA |
| | | | | | | ENESKLL |
| | | | | | | MK |
| 1732.8785 | 1732.8311 | −.0474 | −27 | 114 | 129 | SVGPETA |
| | | | | | | ENESKLL |
| | | | | | | MK |
| 2130.9299 | 2130.75 | −.1799 | −84 | 51 | 68 | LEMHGLW |
| | | | | | | EGSQEGDG |
| | | | | | | WQR |
| 2317.0847 | 2316.875 | −.2097 | −91 | 249 | 268 | ADMSPCP |
| | | | | | | HLPLITP |
| | | | | | | GIC MFK |

| | |
|---|---|
| Tax_Id = 9606 Gene_Symbol = GTF3C5 | IPI00642443 |
| General transcription factor IIIC, | |
| polypeptide 5, 63 kDa | |

Peptide Information

| Calc. | Obsrv. | | | Start | End | |
|---|---|---|---|---|---|---|
| Mass | Mass | ±da | ±ppm | Seq. | Seq. | Sequence |
| 807.4247 | 807.3706 | −.0541 | −67 | 14 | 20 | IYADPTK |
| 963.5258 | 963.5344 | .0086 | 9 | 14 | 21 | IYADPTKR |
| 1221.6951 | 1221.6112 | −.0839 | −69 | 235 | 245 | ANISVH |
| | | | | | | PDKLK |
| 1292.7686 | 1292.6439 | −.1247 | −96 | 41 | 51 | FSTSSL |
| | | | | | | LLRIR |
| 1388.5835 | 1388.6624 | .0789 | 57 | 30 | 40 | DPYCHPVC |
| | | | | | | ANR |
| 1392.7594 | 1392.6851 | −.0743 | −53 | 231 | 243 | NAVKANIS |
| | | | | | | VHPDK |

| | |
|---|---|
| Tax_Id = 9606 Gene_Symbol = | IPI00964988 |
| PPA2 Protein | |

Peptide Information

| Calc. | Obsrv. | | | Start | End | |
|---|---|---|---|---|---|---|
| Mass | Mass | ±da | ±ppm | Seq. | Seq. | Sequence |
| 828.4283 | 828.4123 | −.016 | −19 | 91 | 97 | EPMNPIK |
| 837.4465 | 837.4229 | −.0236 | −28 | 98 | 104 | QYVKDGK |
| 919.5247 | 919.4981 | −.0266 | −29 | 219 | 226 | LYKVPDGK |
| 919.5247 | 919.4981 | −.0266 | −29 | 219 | 226 | LYKVPDGK |
| 988.4734 | 988.4319 | −.0415 | −42 | 196 | 203 | FHDIDDVK |
| 1254.6875 | 1254.6885 | .001 | 1 | 156 | 166 | ILSCGEV |
| | | | | | | IHVK |
| 1254.6875 | 1254.6885 | .001 | 1 | 156 | 166 | ILSCGEV |
| | | | | | | IHVK |
| 1392.6688 | 1392.6851 | .0163 | 12 | 27 | 37 | RAMALY |
| | | | | | | HTEER |

| | |
|---|---|
| Tax_Id = 9606 Gene_Symbol = PACRGL | IPI00909252 |
| PACRG-like protein isoform 2 | |

Peptide Information

| Calc. | Obsrv. | | | Start | End | |
|---|---|---|---|---|---|---|
| Mass | Mass | ±da | ±ppm | Seq. | Seq. | Sequence |
| 851.4469 | 851.448 | .0011 | 1 | 24 | 31 | TSSSTQLK |
| 963.4741 | 963.5344 | .0603 | 63 | 4 | 13 | SEGSGG |
| | | | | | | TQLK |
| 1208.5753 | 1208.5834 | .0081 | 7 | 41 | 52 | SSLSTSS |
| | | | | | | PESAR |
| 1208.5753 | 1208.5834 | .0081 | 7 | 41 | 52 | SSLSTSSP |
| | | | | | | ESAR |
| 1388.6007 | 1388.6624 | .0617 | 44 | 113 | 123 | SKIPTY |
| | | | | | | CSICC |

| | |
|---|---|
| Tax_Id = 9606 | IPI00892717 |
| Gene_Symbol = RASGRP3 | |
| Uncharacterized protein | |

Peptide Information

| Calc. | Obsrv. | | | Start | End | |
|---|---|---|---|---|---|---|
| Mass | Mass | ±da | ±ppm | Seq. | Seq. | Sequence |
| 828.3556 | 828.4123 | .0567 | 68 | 98 | 103 | MTEEFR |
| 855.4215 | 855.3708 | −.0507 | −59 | 55 | 60 | LLCMYR |
| 1146.5798 | 1146.4808 | −.099 | −86 | 72 | 79 | LKICYFMR |
| 1146.5798 | 1146.4808 | −.099 | −86 | 72 | 79 | LKICYFMR |
| 1326.6576 | 1326.7201 | .0625 | 47 | 44 | 54 | WYLSS |
| | | | | | | TELAEK |

| | |
|---|---|
| Tax_Id = 9606 | IPI0089439 |
| Gene_Symbol = RASGRP3 | |
| Uncharacterized protein | |

Peptide Information

| Calc. | Obsrv. | | | Start | End | |
|---|---|---|---|---|---|---|
| Mass | Mass | ±da | ±ppm | Seq. | Seq. | Sequence |
| 828.3556 | 828.4123 | .0567 | 68 | 98 | 103 | MTEEFR |
| 855.4215 | 855.3708 | −.0507 | −59 | 55 | 60 | LLCMYR |
| 1146.5798 | 1146.4808 | −.099 | −86 | 72 | 79 | LKICYFMR |
| 1146.5798 | 1146.4808 | −.099 | −86 | 72 | 79 | LKICYFMR |
| 1326.6576 | 1326.7201 | .0625 | 47 | 44 | 54 | WYLSSTE |
| | | | | | | LAEK |

| | |
|---|---|
| Tax_Id = 9606 Gene_Symbol = KPNA1 | IPI00789357 |
| 7 kDa protein | |

Peptide Information

| Calc. | Obsrv. | | | Start | End | |
|---|---|---|---|---|---|---|
| Mass | Mass | ±da | ±ppm | Seq. | Seq. | Sequence |
| 1217.5112 | 1217.4189 | −.0923 | −76 | 13 | 21 | LCQMEA |
| | | | | | | FMR |
| 1313.6163 | 1313.4952 | −.1211 | −92 | 12 | 21 | KLCQME |
| | | | | | | AFMR |
| 1313.6163 | 1313.4952 | −.1211 | −92 | 12 | 21 | KLCQME |
| | | | | | | AFMR |

| | |
|---|---|
| Tax_Id = 9606 Gene_Symbol = MYCL1 | IPI00643210 |
| protein L-Myc-1 isoform 2 | |

Peptide Information

| Calc. | Obsrv. | | | Start | End | |
|---|---|---|---|---|---|---|
| Mass | Mass | ±da | ±ppm | Seq. | Seq. | Sequence |
| 875.4523 | 875.4897 | .0374 | 43 | 206 | 213 | GPPHGWPK |
| 1292.6859 | 1292.6439 | −.042 | −32 | 111 | 121 | GWGRN |
| | | | | | | YASIIR |
| 1364.598 | 1364.6797 | .0817 | 60 | 2 | 13 | CVCAGCR |
| | | | | | | AAPSR |
| 1388.5835 | 1388.6624 | .0789 | 57 | 122 | 132 | RDCMWSG |
| | | | | | | FSAR |
| 1511.6334 | 1511.7499 | .1165 | 77 | 1 | 13 | MCVCAGC |
| | | | | | | RAAPSR |

| | |
|---|---|
| Tax_Id = 9606 Gene_Symbol = | IPI00792017 |
| SLFN12 Uncharacterized protein | |

Peptide Information

| Calc. | Obsrv. | | | Start | End | |
|---|---|---|---|---|---|---|
| Mass | Mass | ±da | ±ppm | Seq. | Seq. | Sequence |
| 819.3955 | 819.3359 | −.0596 | −73 | 40 | 46 | QNESVSR |
| 875.4581 | 875.4897 | .0316 | 36 | 21 | 28 | VTLGENSR |
| 1069.432 | 1069.3781 | −.0539 | −50 | 61 | 69 | AEIENE |
| | | | | | | DYS |
| 1406.7131 | 1406.6873 | −.0258 | −18 | 47 | 60 | AMCALLN |
| | | | | | | SGGGVIK |

| | |
|---|---|
| Tax_Id = 9606 Gene_Symbol = | IPI00064351 |
| C8orf76 Uncharacterized protein C8orf76 | |

Peptide Information

| Calc. | Obsrv. | | | Start | End | |
|---|---|---|---|---|---|---|
| Mass | Mass | ±da | ±ppm | Seq. | Seq. | Sequence |
| 824.4083 | 824.4028 | −.0055 | −7 | 264 | 270 | ACASFIR |
| 851.437 | 851.448 | .011 | 13 | 67 | 72 | RQEYQK |
| 990.4738 | 990.4394 | −.0344 | −35 | 293 | 300 | TQQEIEDK |
| 1193.5732 | 1193.5586 | −.0146 | −12 | 24 | 34 | RSGPPAS |
| | | | | | | YCAK |
| 1254.6212 | 1254.6885 | .0673 | 54 | 73 | 83 | ALQEYSS |
| | | | | | | ISEK |
| 1254.6212 | 1254.6885 | .0673 | 54 | 73 | 83 | ALQEYSSI |
| | | | | | | SEK |
| 1392.661 | 1392.6851 | .0241 | 17 | 241 | 252 | ALTNIQNC |
| | | | | | | MAEK |

| | |
|---|---|
| Tax_Id = 9606 Gene_Symbol = POR | IPI00852905 |
| Uncharacterized protein | |

Peptide Information

| Calc. | Obsrv. | | | Start | End | |
|---|---|---|---|---|---|---|
| Mass | Mass | ±da | ±ppm | Seq. | Seq. | Sequence |
| 832.3618 | 832.3014 | −.0604 | −73 | 35 | 41 | LPDGDCR |
| 870.5156 | 870.5143 | −.0013 | −1 | 2 | 9 | RLPGVNSK |
| 1404.7417 | 1404.6514 | −.0903 | −64 | 42 | 54 | GVCQPPV |
| | | | | | | QGRPPL |

| | |
|---|---|
| Tax_Id = 9606 Gene_Symbol = ACACB | IPI00848209 |
| ACACB protein | |

Peptide Information

| Calc. | Obsrv. | | | Start | End | |
|---|---|---|---|---|---|---|
| Mass | Mass | ±da | ±ppm | Seq. | Seq. | Sequence |
| 820.3988 | 820.343 | −.0558 | −68 | 839 | 844 | DVSWWK |
| 837.4828 | 837.4229 | −.0599 | −72 | 110 | 116 | TFVQSKK |
| 843.5046 | 843.4919 | −.0127 | −15 | 366 | 372 | DLLQAKR |
| 990.5255 | 990.4394 | −.0861 | −87 | 699 | 706 | EIEFLPSR |
| 1217.4991 | 1217.4189 | −.0802 | −66 | 70 | 79 | TSLYSE |
| | | | | | | DDCK |
| 1254.6113 | 1254.6885 | .0772 | 62 | 443 | 452 | FKTQEY |
| | | | | | | PEGR |
| 1254.6113 | 1254.6885 | .0772 | 62 | 443 | 452 | FKTQEY |
| | | | | | | PEGR |
| 1313.6121 | 1313.4952 | −.1169 | −89 | 147 | 156 | DEFAE |
| | | | | | | DRIYR |
| 1313.6121 | 1313.4952 | −.1169 | −89 | 147 | 156 | DEFAED |
| | | | | | | RIYR |
| 1406.7726 | 1406.6873 | −.0853 | −61 | 713 | 724 | WMLAGR |
| | | | | | | PHPTLK |
| 1529.6989 | 1529.8091 | .1102 | 72 | 825 | 838 | GFSGGM |
| | | | | | | KGCLGT |
| | | | | | | WR |

| | |
|---|---|
| 16/A16 | Instr./Gel Origin |
| [1] Sample Project | Instrument |
| 20110325 | Sample |
| | Name |

| | |
|---|---|
| Protein Name | Accession No. |
| Tax_Id = 9606 Gene_Symbol = | IPI00760788 |

KLHL22 Isoform 2 of Ketch-like protein 22
KH22 (SEQ ID. NO. KH22)

Peptide Information

| Calc. | Obsrv. | | | Start | End | |
|---|---|---|---|---|---|---|
| Mass | Mass | ±da | ±ppm | Seq. | Seq. | Sequence |
| 880.4523 | 880.4501 | −.0022 | −2 | 455 | 461 | RGEDYLK |
| 957.4649 | 957.4094 | −.0555 | −58 | 372 | 378 | YDPRHNR |
| 999.537 | 999.4399 | −.0971 | −97 | 330 | 338 | HFTASLA |
| | | | | | | PR |
| 1056.5078 | 1056.4048 | −.103 | −97 | 448 | 455 | MYITCGRR |
| 1184.606 | 1184.4923 | −.1137 | −96 | 178 | 187 | NFVAFS |
| | | | | | | RTDK |
| 1241.646 | 1241.576 | −.07 | −56 | 484 | 494 | AWHGMAT |
| | | | | | | LLNK |
| 1279.61 | 1279.5404 | −.0696 | −54 | 268 | 278 | DTVASAL |
| | | | | | | MYHR |
| 1328.6667 | 1328.6581 | −.0086 | −6 | 66 | 76 | ILLAASC |
| | | | | | | DYFR |

| | |
|---|---|
| Tax_Id = 9606 Gene_Symbol = - | IPI00746984 |
| Conserved hypothetical protein | |

Peptide Information

| Calc. | Obsrv. | | | Start | End | |
|---|---|---|---|---|---|---|
| Mass | Mass | ±da | ±ppm | Seq. | Seq. | Sequence |
| 999.4788 | 999.4399 | −.0389 | −39 | 40 | 48 | CQGAKH |
| | | | | | | NGK |
| 1083.4814 | 1083.5062 | .0248 | 23 | 45 | 56 | HNGKGGA |
| | | | | | | DAAEG |
| 2210.0586 | 2209.9976 | −.061 | −28 | 5 | 23 | SPDAHHP |
| | | | | | | GLFMFEP |
| | | | | | | ILE EK |

| | |
|---|---|
| Tax_Id = 9606 Gene_Symbol = SEC14L3 | IPI00894175 |
| 5 kDa protein | |

Peptide Information

| | Obsrv. | | | Start | End | |
|---|---|---|---|---|---|---|
| Calc. Mass | Mass | ±da | ±ppm | Seq. | Seq. | Sequence |
| 809.3821 | 809.3715 | −.0106 | −13 | 19 | 25 | MESLSSR |
| 1146.5936 | 1146.4998 | −.0938 | −82 | 1 | 11 | MSGRVGDLSPK |
| 1456.8007 | 1456.7645 | −.0362 | −25 | 5 | 18 | VGDLSPKQAET |
| | | | | | | LAK |
| 1456.8007 | 1456.7645 | −.0362 | −25 | 5 | 18 | VGDLSPKQAET |
| | | | | | | LAK |

| | |
|---|---|
| Tax_Id = 9606 Gene_Symbol = MAPKAPK5 6 kDa | IPI00797302 |
| protein | |

Peptide Information

| | Obsrv. | | | Start | End | |
|---|---|---|---|---|---|---|
| Calc. Mass | Mass | ±da | ±ppm | Seq. | Seq. | Sequence |
| 1142.4341 | 1142.326 | −.1081 | −95 | 2 | 11 | MEEEGGAMEK |
| 1241.4847 | 1241.576 | .0913 | 74 | 1 | 11 | MMEEEGGAMEK |
| 1456.6294 | 1456.7645 | .1351 | 93 | 2 | 14 | MEEEGGAMEK |
| | | | | | | VSK |
| 1456.6294 | 1456.7645 | .1351 | 93 | 2 | 14 | MEEEGGAMEK |
| | | | | | | VSK |

| | |
|---|---|
| Tax_Id = 9606 Gene_Symbol = KRT71 Keratin, | IPI00061200 |
| type II cytoskeletal 71 | |

Peptide Information

| | Obsrv. | | | Start | End | |
|---|---|---|---|---|---|---|
| Calc. Mass | Mass | ±da | ±ppm | Seq. | Seq. | Sequence |
| 800.3686 | 800.3445 | −.0241 | −30 | 66 | 73 | SGGYGFGR |
| 919.4843 | 919.5223 | .038 | 41 | 56 | 65 | SLNVASGSGK |
| 959.4905 | 959.4409 | −.0496 | −52 | 383 | 391 | GDNALKDAR |
| 961.4949 | 961.4734 | −.0215 | −22 | 346 | 353 | NEISELTR |
| 961.4949 | 961.4734 | −.0215 | −22 | 346 | 353 | NEISELTR |
| 997.4697 | 997.4683 | −.0014 | −1 | 497 | 505 | SRGSANDYK |
| 1018.5164 | 1018.4394 | −.077 | −76 | 191 | 199 | QLETLSGDR |
| 1018.5164 | 1018.4394 | −.077 | −76 | 191 | 199 | QLETLSGDR |
| 1146.6113 | 1146.4998 | −.1115 | −97 | 190 | 199 | KQLETLSGDR |
| 1873.0178 | 1872.8617 | −.1561 | −83 | 175 | 190 | NNLEPILEGY |

| | |
|---|---|
| Tax_Id = 9606 Gene_Symbol = SCHIP1 | IPI00947302 |
| Uncharacterized protein | |

Peptide Information

| | Obsrv. | | | Start | End | |
|---|---|---|---|---|---|---|
| Calc. Mass | Mass | ±da | ±ppm | Seq. | Seq. | Sequence |
| 980.4176 | 980.4788 | .0612 | 62 | 25 | 32 | VTPCSECK |
| 1241.6233 | 1241.576 | −.0473 | −38 | 90 | 101 | SPAREPGDVSAR |
| 1312.5612 | 1312.5774 | .0162 | 12 | 2 | 15 | DLGSDAGSSSS |
| | | | | | | SSR |
| 1312.5612 | 1312.5774 | .0162 | 12 | 2 | 15 | DLGSDAGSSSS |
| | | | | | | SSR |
| 1459.5966 | 1459.5133 | −.0833 | −57 | 1 | 15 | MDLGSDAGSS |
| | | | | | | SSSSR |

| | |
|---|---|
| Tax_Id = 9606 Gene_Symbol = KLHL22 Isoform 1 of | IPI00156791 |
| Ketch-like protein 22 | |

Peptide Information

| | Obsrv. | | | Start | End | |
|---|---|---|---|---|---|---|
| Calc. Mass | Mass | ±da | ±ppm | Seq. | Seq. | Sequence |
| 880.4523 | 880.4501 | −.0022 | −2 | 455 | 461 | RGEDYLK |
| 957.4649 | 957.4094 | −.0555 | −58 | 372 | 378 | YDPRHNR |
| 999.537 | 999.4399 | −.0971 | −97 | 330 | 338 | HFTASLAPR |
| 1056.5078 | 1056.4048 | −.103 | −97 | 448 | 455 | MYITCGRR |
| 1184.606 | 1184.4923 | −.1137 | −96 | 178 | 187 | NFVAFSRTDK |
| 1241.646 | 1241.576 | −.07 | −56 | 484 | 494 | AWHGMATLLNK |
| 1279.61 | 1279.5404 | −.0696 | −54 | 268 | 278 | DTVASALMYHR |
| 1328.6667 | 1328.6581 | −.0086 | −6 | 66 | 76 | ILLAASCDYFR |

| | |
|---|---|
| Tax_Id = 9606 Gene_Symbol = HTATSF1 HIV | IPI00013788 |
| Tat-specific factor 1 | |

Peptide Information

| | Obsrv. | | | Start | End | |
|---|---|---|---|---|---|---|
| Calc. Mass | Mass | ±da | ±ppm | Seq. | Seq. | Sequence |
| 831.457 | 831.4774 | .0204 | 25 | 655 | 662 | GLEAADKK |
| 935.3774 | 935.3341 | −.0433 | −46 | 466 | 473 | ESEEGCPK |
| 996.4269 | 996.3946 | −.0323 | −32 | 717 | 724 | LFDDSDER |
| 999.42 | 999.4399 | .0199 | 20 | 475 | 483 | GFEGSCSQK |
| 1056.4667 | 1056.4048 | −.0619 | −59 | 18 | 26 | MQELYGDGK |
| 1083.4478 | 1083.5062 | .0584 | 54 | 637 | 645 | VFDDESDEK |
| 1241.5765 | 1241.576 | −.0005 | 0 | 182 | 191 | GDGLCCYLKR |
| 1383.6764 | 1383.5829 | −.0935 | −68 | 399 | 410 | ARHFSEHPSTSK |
| 1783.7253 | 1783.7095 | −.0158 | −9 | 434 | 450 | TEDGGEFEEGA |
| | | | | | | SENNA K |

| | |
|---|---|
| Tax_Id = 9606 Gene_Symbol = DDX23 Probable | IPI00006725 |
| ATP-dependent RNA helicase DDX23 | |

Peptide Information

| | Obsrv. | | | Start | End | |
|---|---|---|---|---|---|---|
| Calc. Mass | Mass | ±da | ±ppm | Seq. | Seq. | Sequence |
| 831.457 | 831.4774 | .0204 | 25 | 2 | 9 | AGELADKK |
| 996.4819 | 996.3946 | −.0873 | −88 | 588 | 596 | MLANFESGK |
| 1012.4768 | 1012.4625 | −.0143 | −14 | 588 | 596 | MLANFESGK |
| 1018.455 | 1018.4394 | −.0156 | −15 | 319 | 326 | FYGDLMEK |
| 1018.4799 | 1018.4394 | −.0405 | −40 | 12 | 20 | DASPSKEER |
| 1146.6001 | 1146.4998 | −.1003 | −87 | 430 | 441 | DIIGVAETGSGK |
| 1198.5951 | 1198.4972 | −.0979 | −82 | 375 | 385 | EDYSITTKGGK |
| 1241.6195 | 1241.576 | −.0435 | −35 | 643 | 652 | VFLMSESEKR |
| 1456.8119 | 1456.7645 | −.0474 | −33 | 712 | 726 | AGAKDILVATD |
| | | | | | | VAGR |
| 1456.8119 | 1456.7645 | −.0474 | −33 | 712 | 726 | AGAKDILVATDVAGR |
| 1872.9702 | 1872.8617 | −.1085 | −58 | 462 | 478 | IEESDQGPYAII |
| | | | | | | LAPTR |

| | |
|---|---|
| Tax_Id = 9606 Gene_Symbol = VDAC3 6 kDa protein | IPI00973345 |

Peptide Information

| | Obsrv. | | | Start | End | |
|---|---|---|---|---|---|---|
| Calc. Mass | Mass | ±da | ±ppm | Seq. | Seq. | Sequence |
| 1328.561 | 1328.6581 | .0971 | 73 | 2 | 12 | CNTPTYCDLGK |
| 1459.6014 | 1459.5133 | −.0881 | −60 | 1 | 12 | MCNTPTYCDL |
| | | | | | | GK |
| 1745.7656 | 1745.757 | −.0086 | −5 | 1 | 15 | MCNTPTYCDL |
| | | | | | | GKAAK |

| | |
|---|---|
| Tax_Id = 9606 Gene_Symbol = SCHIP1 | IPI00946287 |
| Uncharacterized protein | |

Peptide Information

| | | | | Start | End | |
|---|---|---|---|---|---|---|
| Calc. Mass | Obsrv. Mass | ±da | ±ppm | Seq. | Seq. | Sequence |
| 980.4176 | 980.4788 | .0612 | 62 | 25 | 32 | VTPCSECK |
| 1241.6233 | 1241.576 | −.0473 | −38 | 90 | 101 | SPAREPGDVSAR |
| 1312.5612 | 1312.5774 | .0162 | 12 | 2 | 15 | DLGSDAGSSSS |
| | | | | | | SSR |
| 1312.5612 | 1312.5774 | .0162 | 12 | 2 | 15 | DLGSDAGSSSS |
| | | | | | | SSR |
| 1459.5966 | 1459.5133 | −.0833 | −57 | 1 | 15 | MDLGSDAGSS |
| | | | | | | SSSSR |

| | |
|---|---|
| Tax_Id = 9606 Gene_Symbol = TSEN2 tRNA-splicing | IPI00926682 |
| endonuclease subunit Sen2 isoform 3 | |

Peptide Information

| Calc. | Obsrv. | | | Start | End | |
|---|---|---|---|---|---|---|
| Mass | Mass | ±da | ±ppm | Seq. | Seq. | Sequence |
| 831.4617 | 831.4774 | .0157 | 19 | 223 | 228 | NRLICR |
| 919.4744 | 919.5223 | .0479 | 52 | 394 | 400 | WVSSRER |
| 1069.491 | 1069.3982 | −.0928 | −87 | 144 | 152 | NEEAQV |
| | | | | | | HDK |
| 1140.437 | 1140.3239 | −.1131 | −99 | 374 | 382 | EMESPE |
| | | | | | | CMK |
| 1140.437 | 1140.3239 | −.1131 | −99 | 374 | 382 | EMESPE |
| | | | | | | CMK |
| 1241.6888 | 1241.576 | −.1128 | −91 | 303 | 312 | YGTDLLL |
| | | | | | | YRK |
| 1279.6165 | 1279.5404 | −.0761 | −59 | 53 | 63 | NAEDIEQ |
| | | | | | | LYGK |
| 1312.5331 | 1312.5774 | .0443 | 34 | 374 | 383 | EMESPE |
| | | | | | | CMKR |
| 1312.5331 | 1312.5774 | .0443 | 34 | 374 | 383 | EMESPE |
| | | | | | | CMKR |
| 1328.528 | 1328.6581 | .1301 | 98 | 374 | 383 | EMESPE |
| | | | | | | CMKR |

| | |
|---|---|
| Tax_Id = 9606 Gene_Symbol = | IPI00657872 |
| IGF2AS 14 kDa protein | |

Peptide Information

| | Obsrv. | | | Start | End | |
|---|---|---|---|---|---|---|
| Calc. Mass | Mass | ±da | ±ppm | Seq. | Seq. | Sequence |
| 961.4632 | 961.4734 | .0102 | 11 | 73 | 81 | EAGGCVRGR |
| 961.481 | 961.4734 | −.0076 | −8 | 53 | 61 | AESRSGAQR |
| 1326.6696 | 1326.5909 | −.0787 | −59 | 41 | 52 | RAPAPAGQQQ |
| | | | | | | MR |

| | |
|---|---|
| Tax_Id = 9606 Gene_Symbol = STK19 | IPI00945765 |
| Uncharacterized protein | |

Peptide Information

| | Obsrv. | | | Start | End | |
|---|---|---|---|---|---|---|
| Calc. Mass | Mass | ±da | ±ppm | Seq. | Seq. | Sequence |
| 809.4152 | 809.3715 | −.0437 | −54 | 24 | 32 | GGGGVSFTK |
| 1184.5994 | 1184.4923 | −.1071 | −90 | 53 | 61 | ACPWREPIR |
| 1326.6649 | 1326.5909 | −.074 | −56 | 33 | 45 | EVDTNVATGAP |
| | | | | | | PR |
| 1328.6052 | 1328.6581 | .0529 | 40 | 83 | 93 | HCSAPEDPIFR |

| | |
|---|---|
| Tax_Id = 9606 Gene_Symbol = DDX23 cDNA FLJ46571 | IPI00903057 |
| fis, clone THYMU3041428, highly similar to | |
| Probable ATP-dependent RNA helicase DDX23 | |

Peptide Information

| | Obsrv. | | | Start | End | |
|---|---|---|---|---|---|---|
| Calc. Mass | Mass | ±da | ±ppm | Seq. | Seq. | Sequence |
| 831.457 | 831.4774 | .0204 | 25 | 2 | 9 | AGELADKK |
| 996.4819 | 996.3946 | −.0873 | −88 | 568 | 576 | MLANFESGK |
| 1012.4768 | 1012.4625 | −.0143 | −14 | 568 | 576 | MLANFESGK |
| 1018.455 | 1018.4394 | −.0156 | −15 | 299 | 306 | FYGDLMEK |
| 1018.4799 | 1018.4394 | −.0405 | −40 | 12 | 20 | DASPSKEER |
| 1146.6001 | 1146.4998 | −.1003 | −87 | 410 | 421 | DIIGVAETGS |
| | | | | | | GK |
| 1198.5951 | 1198.4972 | −.0979 | −82 | 355 | 365 | EDYSITTKGGK |
| 1241.6195 | 1241.576 | −.0435 | −35 | 623 | 632 | VFLMSESEKR |
| 1456.8119 | 1456.7645 | −.0474 | −33 | 692 | 706 | AGAKDILVAT |
| | | | | | | DVAGR |
| 1456.8119 | 1456.7645 | −.0474 | −33 | 692 | 706 | AGAKDILVAT |
| | | | | | | DVAGR |
| 1872.9702 | 1872.8617 | −.1085 | −58 | 442 | 458 | IEESDQGPYA |
| | | | | | | IILAPTR |

| | |
|---|---|
| Tax_Id = 9606 Gene_Symbol = SNX7 Isoform 1 of | IPI00942419 |
| Sorting nexin-7 | |

Peptide Information

| | | | | Start | End | |
|---|---|---|---|---|---|---|
| Calc. Mass | Obsrv. Mass | ±da | ±ppm | Seq. | Seq. | Sequence |
| 896.3964 | 896.4366 | .0402 | 45 | 252 | 258 | CCKATEK |
| 935.4251 | 935.3341 | −.091 | −97 | 244 | 251 | DVASCIDR |
| 961.3971 | 961.4734 | .0763 | 79 | 216 | 222 | EYFDEMK |
| 961.3971 | 961.4734 | .0763 | 79 | 216 | 222 | EYFDEMK |
| 1018.4985 | 1018.4394 | −.0591 | −58 | 323 | 331 | VECANNALK |
| 1018.4985 | 1018.4394 | −.0591 | −58 | 323 | 331 | VECANNALK |
| 1146.5637 | 1146.4998 | −.0639 | −56 | 289 | 298 | DQIQAELDSK |
| 1312.6281 | 1312.5774 | −.0507 | −39 | 109 | 118 | FNDDFIETRR |
| 1312.6281 | 1312.5774 | −.0507 | −39 | 109 | 118 | FNDDFIETRR |
| 1383.5814 | 1383.5829 | .0015 | 1 | 244 | 254 | DVASCIDRCCK |

| | |
|---|---|
| Tax_Id = 9606 Gene_Symbol = HSP90AB5P Heat shock | IPI00556622 |
| protein 90Be | |

Peptide Information

| | Obsrv. | | | Start | End | |
|---|---|---|---|---|---|---|
| Calc. Mass | Mass | ±da | ±ppm | Seq. | Seq. | Sequence |
| 847.3511 | 847.329 | −.0221 | −26 | 1 | 7 | MGYMMAK |
| 919.4367 | 919.5223 | .0856 | 93 | 26 | 33 | AEVDKDDK |
| 959.451 | 959.4409 | −.0101 | −11 | 1 | 8 | MGYMMAKK |

| | |
|---|---|
| Tax_Id = 9606 Gene_Symbol = | IPI00888272 |
| LOC100131519 similar to hCG1651476 | |

Peptide Information

| Calc. | Obsrv. | | | Start | End | |
|---|---|---|---|---|---|---|
| Mass | Mass | ±da | ±ppm | Seq. | Seq. | Sequence |
| 831.4431 | 831.4774 | .0343 | 41 | 37 | 43 | LDARNSR |
| 997.5214 | 997.4683 | −.0531 | −53 | 6 | 12 | YRDLQFR |
| 1083.4636 | 1083.5062 | .0426 | 39 | 13 | 21 | DRDCSG |
| | | | | | | AFR |
| 1421.6447 | 1421.5438 | −.1009 | −71 | 100 | 110 | CKCVPQ |
| | | | | | | MQQSR |

| | |
|---|---|
| Tax_Id = 9606 Gene_Symbol = ZNF483 | IPI00478644 |
| Zinc finger protein 483 | |

Peptide Information

| Calc. | Obsrv. | | | Start | End | |
|---|---|---|---|---|---|---|
| Mass | Mass | ±da | ±ppm | Seq. | Seq. | Sequence |
| 919.4367 | 919.5223 | .0856 | 93 | 145 | 153 | EEAGDI |
| | | | | | | ASK |
| 1037.4647 | 1037.4038 | −.0609 | −59 | 43 | 52 | GNAADA |
| | | | | | | ESFR |
| 1350.6383 | 1350.5377 | −.1006 | −74 | 145 | 157 | EEAGDIA |
| | | | | | | SKSESK |
| 1421.6332 | 1421.5438 | −.0894 | −63 | 168 | 178 | EESEVRS |
| | | | | | | WDWW |

| | |
|---|---|
| Tax_Id = 9606 Gene_Symbol = MTHFS | IPI00643098 |
| 8 kDa protein | |

Peptide Information

| Calc. | Obsrv. | | | Start | End | |
|---|---|---|---|---|---|---|
| Mass | Mass | ±da | ±ppm | Seq. | Seq. | Sequence |
| 809.4489 | 809.3715 | −.0774 | −96 | 9 | 15 | HGNRLGR |
| 880.4233 | 880.4501 | .0268 | 30 | 1 | 8 | MPGLGFDK |
| 1328.6528 | 1328.6581 | .0053 | 4 | 1 | 12 | MPGLGFDK |
| | | | | | | HGNR |

| | |
|---|---|
| 18/A18 | Instr./Gel Origin |
| [1] Sample Project | Instrument |
| 20110325 | Sample |
| | Name |

| | |
|---|---|
| Protein Name | Accession No. |
| Tax_Id = 9606 Gene_Symbol = | IPI00917278 |
| SETD2 120 kDa protein | |

KH23 (SEQ ID. NO. KH23) Peptide Information

| Calc. | Obsrv. | | | Start | End | |
|---|---|---|---|---|---|---|
| Mass | Mass | ±da | ±ppm | Seq. | Seq. | Sequence |
| 818.4042 | 818.405 | .0008 | 1 | 608 | 613 | HLYEEK |
| 818.4042 | 818.405 | .0008 | 1 | 608 | 613 | HLYEEK |
| 829.4315 | 829.4913 | .0598 | 72 | 953 | 958 | DPQRWK |
| 891.4418 | 891.4531 | .0113 | 13 | 18 | 25 | TSENEAIK |
| 980.5047 | 980.4961 | −.0086 | −9 | 885 | 892 | DFSKNLEK |
| 1018.5779 | 1018.5897 | .0118 | 12 | 148 | 156 | ELDSLS |
| | | | | | | KVK |
| 1018.5779 | 1018.5897 | .0118 | 12 | 148 | 156 | ELDSLS |
| | | | | | | KVK |
| 1047.5615 | 1047.557 | −.0045 | −4 | 94 | 102 | GSELR |
| | | | | | | MINK |
| 1091.646 | 1091.5599 | −.0861 | −79 | 853 | 861 | FLLSL |
| | | | | | | QKDK |
| 1102.6508 | 1102.5885 | −.0623 | −57 | 126 | 134 | KLDEL |
| | | | | | | PIFK |
| 1103.5844 | 1103.588 | .0036 | 3 | 414 | 423 | SSEFLKH |
| | | | | | | AGK |

| | |
|---|---|
| Tax_Id = 9606 Gene_Symbol = CCBL2 | IPI00553183 |
| kynurenine--oxoglutarate transaminase | |
| 3 isoform 2 | |

Peptide Information

| Calc. | Obsrv. | | | Start | End | |
|---|---|---|---|---|---|---|
| Mass | Mass | ±da | ±ppm | Seq. | Seq. | Sequence |
| 830.4341 | 830.4764 | .0423 | 51 | 366 | 371 | WMTKHK |
| 908.52 | 908.4479 | −.0721 | −79 | 2 | 9 | SLKFTNAK |
| 955.507 | 955.4167 | −.0903 | −95 | 363 | 369 | FVKWMTK |
| 986.5193 | 986.4275 | −.0918 | −93 | 75 | 82 | ALSYLYEK |
| 1040.5524 | 1040.5715 | .0191 | 18 | 388 | 395 | SQFEKFVR |
| 1042.4476 | 1042.4867 | .0391 | 38 | 355 | 362 | NNEPYDYK |
| 1055.5554 | 1055.5176 | −.0378 | −36 | 1 | 9 | MSLKFT |
| | | | | | | NAK |
| 1116.6023 | 1116.5959 | −.0064 | −6 | 393 | 400 | FVRFCF |
| | | | | | | IK |
| 1623.8047 | 1623.7913 | −.0134 | −8 | 373 | 387 | LSAIPVSA |
| | | | | | | FCNSETK |
| 1753.9484 | 1753.8547 | −.0937 | −53 | 238 | 254 | TITIGSAG |
| | | | | | | KTFSVTG |
| | | | | | | WK |
| 1753.9484 | 1753.8547 | −.0937 | −53 | 238 | 254 | TITIGSAG |
| | | | | | | KTFSVTG |
| | | | | | | WK |

| | |
|---|---|
| Tax_Id = 9606 Gene_Symbol = SPTBN5 | IPI00219168 |
| Spectrin beta chain, brain 4 | |

Peptide Information

| | | | | Start | | |
|---|---|---|---|---|---|---|
| Calc. Mass | Obsrv.Mass | ±da | ±ppm | Seq. | End Seq. | Sequence |
| 814.4893 | 814.5114 | .0221 | 27 | 1646 | 1652 | RPLVSSR |
| 818.4254 | 818.405 | −.0204 | −25 | 2372 | 2378 | ELDNVTK |
| 818.4254 | 818.405 | −.0204 | −25 | 2372 | 2378 | ELDNVTK |
| 829.5142 | 829.4913 | −.0229 | −28 | 1347 | 1353 | NILQTLK |
| 830.4519 | 830.4764 | .0245 | 30 | 1195 | 1200 | VLWEQR |
| 856.5251 | 856.5228 | −.0023 | −3 | 1256 | 1263 | LLSTLGPR |
| 877.4196 | 877.437 | .0174 | 20 | 3271 | 3277 | LQTEACR |
| 877.5002 | 877.437 | −.0632 | −72 | 3544 | 3551 | QHLLPGGR |
| 891.4539 | 891.4531 | −.0008 | −1 | 2657 | 2663 | CQAMLLR |
| 908.5312 | 908.4479 | −.0833 | −92 | 1036 | 1043 | HALQLAQK |
| 923.4806 | 923.4602 | −.0204 | −22 | 1571 | 1578 | AHQGQVQR |
| 923.5057 | 923.4602 | −.0455 | −49 | 10 | 18 | ELLGAAGHR |
| 980.473 | 980.4961 | .0231 | 24 | 1 | 9 | MAGQPHSPR |
| 983.5057 | 983.431 | −.0747 | −76 | 2235 | 2241 | KHWEDLR |
| 986.4901 | 986.4275 | −.0626 | −63 | 2326 | 2333 | NRDPEEVK |
| 1002.4963 | 1002.4139 | −.0824 | −82 | 2248 | 2255 | GQELEDRR |
| 1012.5323 | 1012.4543 | −.078 | −77 | 3180 | 3187 | YPHIQAQR |
| 1016.5483 | 1016.5704 | .0221 | 22 | 507 | 514 | RQEEVTVR |
| 1042.564 | 1042.4867 | −.0773 | −74 | 1857 | 1866 | ATSLPNNVAR |
| 1057.5571 | 1057.5472 | −.0099 | −9 | 3663 | 3671 | DARPGCLLR |
| 1057.5571 | 1057.5472 | −.0099 | −9 | 3663 | 3671 | DARPGCLLR |
| 1085.5698 | 1085.4945 | −.0753 | −69 | 1086 | 1094 | QVQEQVAQR |
| 1089.4994 | 1089.5704 | .071 | 65 | 2491 | 2500 | AQMDTSPAPR |
| 1089.4994 | 1089.5704 | .071 | 65 | 2491 | 2500 | AQMDTSPAPR |
| 1103.6307 | 1103.588 | −.0427 | −39 | 2316 | 2325 | SISDLSLQLK |
| 1109.5231 | 1109.5684 | .0453 | 41 | 2588 | 2595 | MERWLCSK |
| 1136.6171 | 1136.5674 | −.0497 | −44 | 1368 | 1377 | HVEALQQVGR |
| 1142.6277 | 1142.5398 | −.0879 | −77 | 373 | 382 | LQTALQAQNR |
| 1217.6195 | 1217.6008 | −.0187 | −15 | 2384 | 2394 | EALIQALDCGK |
| 1230.6688 | 1230.7039 | .0351 | 29 | 3635 | 3647 | ALGSTAAQSL |
| | | | | | | SPK |
| 1442.8101 | 1442.7466 | −.0635 | −44 | 2061 | 2073 | LEEILAAQEVS |
| | | | | | | LK |
| 1753.9205 | 1753.8547 | −.0658 | −38 | 2 | 18 | AGQPHSPREL |
| | | | | | | LGAAGH R |
| 1753.9205 | 1753.8547 | −.0658 | −38 | 2 | 18 | AGQPHSPREL |
| | | | | | | LGAAGHR |

| | |
|---|---|
| Tax_Id = 9606 Gene_Symbol = CCBL2 | IPI00465373 |
| Isoform 1 of Kynurenine--oxoglutarate | |
| transaminase 3 | |

Peptide Information

| Calc. | Obsrv. | | | Start | End | |
|---|---|---|---|---|---|---|
| Mass | Mass | ±da | ±ppm | Seq. | Seq. | Sequence |
| 830.4341 | 830.4764 | .0423 | 51 | 400 | 405 | WMTKHK |
| 923.5197 | 923.4602 | −.0595 | −64 | 26 | 34 | ILGFSTS |
| | | | | | | AK |
| 923.5197 | 923.4602 | −.0595 | −64 | 26 | 34 | ILGFSTS |
| | | | | | | AK |
| 955.507 | 955.4167 | −.0903 | −95 | 397 | 403 | FVKWMTK |
| 986.5193 | 986.4275 | −.0918 | −93 | 109 | 116 | ALSYLYEK |
| 1040.5524 | 1040.5715 | .0191 | 18 | 422 | 429 | SQFEKFVR |
| 1042.4476 | 1042.4867 | .0391 | 38 | 389 | 396 | NNEPYDYK |
| 1055.5554 | 1055.5176 | −.0378 | −36 | 35 | 43 | MSLKFT |
| | | | | | | NAK |
| 1116.6023 | 1116.5959 | −.0064 | −6 | 427 | 434 | FVRFCFIK |
| 1623.8047 | 1623.7913 | −.0134 | −8 | 407 | 421 | LSAIPVSA |
| | | | | | | FCNSETK |
| 1753.9484 | 1753.8547 | −.0937 | −53 | 272 | 288 | TITIGSAG |
| | | | | | | KTFSVTG |
| | | | | | | WK |
| 1753.9484 | 1753.8547 | −.0937 | −53 | 272 | 288 | TITIGSAG |
| | | | | | | KTFSVTG |
| | | | | | | WK |

| | |
|---|---|
| Tax_Id = 9606 Gene_Symbol = ARHGAP29 | IPI00152011 |
| Isoform 1 of Rho GTPase-activating | |
| protein 29 | |

Peptide Information

| Calc. | Obsrv. | | | Start | End | |
|---|---|---|---|---|---|---|
| Mass | Mass | ±da | ±ppm | Seq. | Seq. | Sequence |
| 818.4553 | 818.405 | −.0503 | −61 | 708 | 714 | VCGNKIK |
| 818.4553 | 818.405 | −.0503 | −61 | 708 | 714 | VCGNKIK |
| 932.5597 | 932.4811 | −.0786 | −84 | 85 | 92 | VLKSIMNK |
| 950.436 | 950.4933 | .0573 | 60 | 298 | 304 | NEMEKQR |
| 1003.4955 | 1003.3978 | −.0977 | −97 | 1050 | 1058 | NPAFEG |
| | | | | | | VNR |
| 1018.5237 | 1018.5897 | .066 | 65 | 318 | 326 | MLEAEN |
| | | | | | | ALK |
| 1018.5237 | 1018.5897 | .066 | 65 | 318 | 326 | MLEAEN |
| | | | | | | ALK |
| | 1018.5897 | | | | | |
| 1040.5007 | 1040.5715 | .0708 | 68 | 828 | 836 | VVDHAE |
| | | | | | | ENK |
| 1055.464 | 1055.5176 | .0536 | 51 | 947 | 955 | ATSFEE |
| | | | | | | SER |
| 1057.6517 | 1057.5472 | −.1045 | −99 | 289 | 297 | FVQPLL |
| | | | | | | GRK |
| 1057.6517 | 1057.5472 | −.1045 | −99 | 289 | 297 | FVQPLL |
| | | | | | | GRK |
| 1091.515 | 1091.5599 | .0449 | 41 | 690 | 698 | ICASEI |
| | | | | | | ENR |
| 1102.6255 | 1102.5885 | −.037 | −34 | 221 | 229 | NIVSWV |
| | | | | | | EKK |
| 1109.6313 | 1109.5684 | −.0629 | −57 | 1025 | 1034 | LLLASP |
| | | | | | | PNER |
| 1116.5031 | 1116.5959 | .0928 | 83 | 64 | 72 | EAIFSD |
| | | | | | | CFK |

| | |
|---|---|
| Tax_Id = 9606 Gene_Symbol = SNX7 | IPI00942419 |
| Isoform 1 of Sorting nexin-7 | |

Peptide Information

| Calc. | Obsrv. | | | Start | End | |
|---|---|---|---|---|---|---|
| Mass | Mass | ±da | ±ppm | Seq. | Seq. | Sequence |
| 896.3964 | 896.4366 | .0402 | 45 | 252 | 258 | CCKATEK |
| 935.4251 | 935.3341 | −.091 | −97 | 244 | 251 | DVASCIDR |
| 961.3971 | 961.4734 | .0763 | 79 | 216 | 222 | EYFDEMK |
| 961.3971 | 961.4734 | .0763 | 79 | 216 | 222 | EYFDEMK |
| 1018.4985 | 1018.4394 | −.0591 | −58 | 323 | 331 | VECANN |
| | | | | | | ALK |
| 1018.4985 | 1018.4394 | −.0591 | −58 | 323 | 331 | VECANN |
| | | | | | | ALK |
| 1146.5637 | 1146.4998 | −.0639 | −56 | 289 | 298 | DQIQAEL |
| | | | | | | DSK |
| 1312.6281 | 1312.5774 | −.0507 | −39 | 109 | 118 | FNDDFIE |
| | | | | | | TRR |
| 1312.6281 | 1312.5774 | −.0507 | −39 | 109 | 118 | FNDDFIE |
| | | | | | | TRR |
| 1383.5814 | 1383.5829 | .0015 | 1 | 244 | 254 | DVASCIDR |
| | | | | | | CCK |

| | |
|---|---|
| Tax_Id = 9606 Gene_Symbol = HSP90AB5P | IPI00556622 |
| Heat shock protein 90Be | |

Peptide Information

| | Obsrv. | | | Start | End | |
|---|---|---|---|---|---|---|
| Calc. Mass | Mass | ±da | ±ppm | Seq. | Seq. | Sequence |
| 847.3511 | 847.329 | −.0221 | −26 | 1 | 7 | MGYMMAK |
| 919.4367 | 919.5223 | .0856 | 93 | 26 | 33 | AEVDKDDK |
| 959.451 | 959.4409 | −.0101 | −11 | 1 | 8 | MGYMMAKK |

| | |
|---|---|
| Tax_Id = 9606 Gene_Symbol = | IPI00888272 |
| LOC100131519 similar to hCG1651476 | |

Peptide Information

| Calc. | Obsrv. | | | Start | End | |
|---|---|---|---|---|---|---|
| Mass | Mass | ±da | ±ppm | Seq. | Seq. | Sequence |
| 831.4431 | 831.4774 | .0343 | 41 | 37 | 43 | LDARNSR |
| 997.5214 | 997.4683 | −.0531 | −53 | 6 | 12 | YRDLQFR |
| 1083.4636 | 1083.5062 | .0426 | 39 | 13 | 21 | DRDCSG |
| | | | | | | AFR |
| 1421.6447 | 1421.5438 | −.1009 | −71 | 100 | 110 | CKCVPQMQ |
| | | | | | | QSR |

| | |
|---|---|
| Tax_Id = 9606 Gene_Symbol = ZNF483 | IPI00478644 |
| Zinc finger protein 483 | |

Peptide Information

| Calc. | Obsrv. | | | Start | End | |
|---|---|---|---|---|---|---|
| Mass | Mass | ±da | ±ppm | Seq. | Seq. | Sequence |
| 919.4367 | 919.5223 | .0856 | 93 | 145 | 153 | EEAGDI |
| ASK | | | | | | |
| 1037.4647 | 1037.4038 | −.0609 | −59 | 43 | 52 | GNAADA |
| ESFR | | | | | | |
| 1350.6383 | 1350.5377 | −.1006 | −74 | 145 | 157 | EEAGDIAS |
| | | | | | | KSESK |
| 1421.6332 | 1421.5438 | −.0894 | −63 | 168 | 178 | EESEVRS |

| | |
|---|---|
| Tax_Id = 9606 Gene_Symbol = MTHFS | IPI00643098 |
| 8 kDa protein | |

Peptide Information

| Calc. | Obsrv. | | | Start | End | |
|---|---|---|---|---|---|---|
| Mass | Mass | ±da | ±ppm | Seq. | Seq. | Sequence |
| 809.4489 | 809.3715 | −.0774 | −96 | 9 | 15 | HGNRLGR |
| 880.4233 | 880.4501 | .0268 | 30 | 1 | 8 | MPGLGFDK |
| 1328.6528 | 1328.6581 | .0053 | 4 | 1 | 12 | MPGLGFD |
| | | | | | | KHGNR |

| | |
|---|---|
| 18/A18 | Instr./Gel Origin |
| [1] Sample Project | Instrument |
| 20110325 | Sample |
| | Name |

| | |
|---|---|
| Protein Name | Accession No. |
| Tax_Id = 9606 Gene_Symbol = | IPI00917278 |
| SETD2 120 kDa protein | |

Peptide Information

| Calc. | Obsrv. | | | Start | End | |
|---|---|---|---|---|---|---|
| Mass | Mass | ±da | ±ppm | Seq. | Seq. | Sequence |
| 818.4042 | 818.405 | .0008 | 1 | 608 | 613 | HLYEEK |
| 818.4042 | 818.405 | .0008 | 1 | 608 | 613 | HLYEEK |
| 829.4315 | 829.4913 | .0598 | 72 | 953 | 958 | DPQRWK |
| 891.4418 | 891.4531 | .0113 | 13 | 18 | 25 | TSENEAIK |
| 980.5047 | 980.4961 | −.0086 | −9 | 885 | 892 | DFSKNLEK |
| 1018.5779 | 1018.5897 | .0118 | 12 | 148 | 156 | ELDSLS |
| | | | | | | KVK |
| 1018.5779 | 1018.5897 | .0118 | 12 | 148 | 156 | ELDSLS |
| | | | | | | KVK |
| 1047.5615 | 1047.557 | −.0045 | −4 | 94 | 102 | GSELRM |
| | | | | | | INK |
| 1091.646 | 1091.5599 | −.0861 | −79 | 853 | 861 | FLLSLQ |
| | | | | | | KDK |
| 1102.6508 | 1102.5885 | −.0623 | −57 | 126 | 134 | KLDELP |
| | | | | | | IFK |
| 1103.5844 | 1103.588 | .0036 | 3 | 414 | 423 | SSEFLKH |
| | | | | | | AGK |

| | |
|---|---|
| Tax_Id = 9606 Gene_Symbol = CCBL2 | IPI00553183 |
| kynurenine--oxoglutarate transaminase | |
| 3 isoform 2 | |

Peptide Information

| Calc. | Obsrv. | | | Start | End | |
|---|---|---|---|---|---|---|
| Mass | Mass | ±da | ±ppm | Seq. | Seq. | Sequence |
| 830.4341 | 830.4764 | .0423 | 51 | 366 | 371 | WMTKHK |
| 908.52 | 908.4479 | −.0721 | −79 | 2 | 9 | SLKFTNAK |
| 955.507 | 955.4167 | −.0903 | −95 | 363 | 369 | FVKWMTK |
| 986.5193 | 986.4275 | −.0918 | −93 | 75 | 82 | ALSYLYEK |
| 1040.5524 | 1040.5715 | .0191 | 18 | 388 | 395 | SQFEKFVR |
| 1042.4476 | 1042.4867 | .0391 | 38 | 355 | 362 | NNEPYDYK |
| 1055.5554 | 1055.51 | — | −36 | 1 | 9 | MSLKF |
| 1116.6023 | 1116.59 | — | — | 393 | 40 | FVRFC |
| 1623.8047 | 1623.79 | — | — | 373 | 38 | LSAIPVS |
| | | | | | | AFC |
| 1753.9484 | 1753.85 | — | −53 | 238 | 25 | TITIGSAG |
| | | | | | | KTF |
| 1753.9484 | 1753.85 | — | −53 | 238 | 25 | TITIGSAG |
| | | | | | | KTF |

| | |
|---|---|
| Tax_Id = 9606 Gene_Symbol = SPTBN5 | IPI00219168 |
| Spectrin beta chain, brain 4 | |

Peptide Information

| | Obsrv. | | | Start | End | |
|---|---|---|---|---|---|---|
| Calc. Mass | Mass | ±da | ±ppm | Seq. | Seq. | Sequence |
| 814.4893 | 814.5114 | .0221 | 27 | 1646 | 1652 | RPLVSSR |
| 818.4254 | 818.405 | −.0204 | −25 | 2372 | 2378 | ELDNVTK |
| 818.4254 | 818.405 | −.0204 | −25 | 2372 | 2378 | ELDNVTK |
| 829.5142 | 829.4913 | −.0229 | −28 | 1347 | 1353 | NILQTLK |
| 830.4519 | 830.4764 | .0245 | 30 | 1195 | 1200 | VLWEQR |
| 856.5251 | 856.5228 | −.0023 | −3 | 1256 | 1263 | LLSTLGPR |
| 877.4196 | 877.437 | .0174 | 20 | 3271 | 3277 | LQTEACR |
| 877.5002 | 877.437 | −.0632 | −72 | 3544 | 3551 | QHLLPGGR |
| 891.4539 | 891.4531 | −.0008 | −1 | 2657 | 2663 | CQAMLLR |
| 908.5312 | 908.4479 | −.0833 | −92 | 1036 | 1043 | HALQLAQK |
| 923.4806 | 923.4602 | −.0204 | −22 | 1571 | 1578 | AHQGQVQR |
| 923.5057 | 923.4602 | −.0455 | −49 | 10 | 18 | ELLGAAGHR |
| 980.473 | 980.4961 | .0231 | 24 | 1 | 9 | MAGQPHSPR |
| 983.5057 | 983.431 | −.0747 | −76 | 2235 | 2241 | KHWEDLR |
| 986.4901 | 986.4275 | −.0626 | −63 | 2326 | 2333 | NRDPEEVK |
| 1002.4963 | 1002.4139 | −.0824 | −82 | 2248 | 2255 | GQELEDRR |
| 1012.5323 | 1012.4543 | −.078 | −77 | 3180 | 3187 | YPHIQAQR |
| 1016.5483 | 1016.5704 | .0221 | 22 | 507 | 514 | RQEEVTVR |
| 1042.564 | 1042.4867 | −.0773 | −74 | 1857 | 1866 | ATSLPNNVAR |
| 1057.5571 | 1057.5472 | −.0099 | −9 | 3663 | 3671 | DARPGCLLR |
| 1057.5571 | 1057.5472 | −.0099 | −9 | 3663 | 3671 | DARPGCLLR |
| 1085.5698 | 1085.4945 | −.0753 | −69 | 1086 | 1094 | QVQEQVAQR |
| 1089.4994 | 1089.5704 | .071 | 65 | 2491 | 2500 | AQMDTSPAPR |
| 1089.4994 | 1089.5704 | .071 | 65 | 2491 | 2500 | AQMDTSPAPR |
| 1103.6307 | 1103.588 | −.0427 | −39 | 2316 | 2325 | SISDLSLQLK |
| 1109.5231 | 1109.5684 | .0453 | 41 | 2588 | 2595 | MERWLCSK |
| 1136.6171 | 1136.5674 | −.0497 | −44 | 1368 | 1377 | HVEALQQVGR |
| 1142.6277 | 1142.5398 | −.0879 | −77 | 373 | 382 | LQTALQAQNR |
| 1217.6195 | 1217.6008 | −.0187 | −15 | 2384 | 2394 | EALIQALDCGK |
| 1230.6688 | 1230.7039 | .0351 | 29 | 3635 | 3647 | ALGSTAAQSL |
| | | | | | | SPK |
| 1442.8101 | 1442.7466 | −.0635 | −44 | 2061 | 2073 | LEEILAAQEVS |
| | | | | | | LK |
| 1753.9205 | 1753.8547 | −.0658 | −38 | 2 | 18 | AGQPHSPREL |
| | | | | | | LGAAGH R |
| 1753.9205 | 1753.8547 | −.0658 | −38 | 2 | 18 | AGQPHSPREL |
| | | | | | | LGAAGHR |

| | |
|---|---|
| Tax_Id = 9606 Gene_Symbol = CCBL2 Isoform 1 of | IPI00465373 |
| Kynurenine--oxoglutarate transaminase 3 | |

Peptide Information

| | Obsrv. | | | Start | End | |
|---|---|---|---|---|---|---|
| Calc. Mass | Mass | ±da | ±ppm | Seq. | Seq. | Sequence |
| 830.4341 | 830.4764 | .0423 | 51 | 400 | 405 | WMTKHK |
| 923.5197 | 923.4602 | −.0595 | −64 | 26 | 34 | ILGFSTSAK |
| 923.5197 | 923.4602 | −.0595 | −64 | 26 | 34 | ILGFSTSAK |
| 955.507 | 955.4167 | −.0903 | −95 | 397 | 403 | FVKWMTK |
| 986.5193 | 986.4275 | −.0918 | −93 | 109 | 116 | ALSYLYEK |
| 1040.5524 | 1040.5715 | .0191 | 18 | 422 | 429 | SQFEKFVR |
| 1042.4476 | 1042.4867 | .0391 | 38 | 389 | 396 | NNEPYDYK |
| 1055.5554 | 1055.5176 | −.0378 | −36 | 35 | 43 | MSLKFTNAK |
| 1116.6023 | 1116.5959 | −.0064 | −6 | 427 | 434 | FVRFCFIK |
| 1623.8047 | 1623.7913 | −.0134 | −8 | 407 | 421 | LSAIPVSAFCNS |
| | | | | | | ETK |
| 1753.9484 | 1753.8547 | −.0937 | −53 | 272 | 288 | TITIGSAGKTFS |
| | | | | | | VTGWK |
| 1753.9484 | 1753.8547 | −.0937 | −53 | 272 | 288 | TITIGSAGKTFS |
| | | | | | | VTGWK |

| | |
|---|---|
| Tax_Id = 9606 Gene_Symbol = ARHGAP29 Isoform 1 of | IP100152011 |
| Rho GTPase-activating protein 29 | |

Peptide Information

| | Obsrv. | | | Start | End | |
|---|---|---|---|---|---|---|
| Calc. Mass | Mass | ±da | ±ppm | Seq. | Seq. | Sequence |
| 818.4553 | 818.405 | −.0503 | −61 | 708 | 714 | VCGNKIK |
| 818.4553 | 818.405 | −.0503 | −61 | 708 | 714 | VCGNKIK |
| 932.5597 | 932.4811 | −.0786 | −84 | 85 | 92 | VLKSIMNK |
| 950.436 | 950.4933 | .0573 | 60 | 298 | 304 | NEMEKQR |
| 1003.4955 | 1003.3978 | −.0977 | −97 | 1050 | 1058 | NPAFEGVNR |
| 1018.5237 | 1018.5897 | .066 | 65 | 318 | 326 | MLEAENALK |
| 1018.5237 | 1018.5897 | .066 | 65 | 318 | 326 | MLEAENALK |
| | 1018.5897 | | | | | |
| 1040.5007 | 1040.5715 | .0708 | 68 | 828 | 836 | VVDHAEENK |
| 1055.464 | 1055.5176 | .0536 | 51 | 947 | 955 | ATSFEESER |
| 1057.6517 | 1057.5472 | −.1045 | −99 | 289 | 297 | FVQPLLGRK |
| 1057.6517 | 1057.5472 | −.1045 | −99 | 289 | 297 | FVQPLLGRK |
| 1091.515 | 1091.5599 | .0449 | 41 | 690 | 698 | ICASEIENR |
| 1102.6255 | 1102.5885 | −.037 | −34 | 221 | 229 | NIVSWVEKK |
| 1109.6313 | 1109.5684 | −.0629 | −57 | 1025 | 1034 | LLLASPPNER |
| 1116.5031 | 1116.5959 | .0928 | 83 | 64 | 72 | EAIFSDCFK |

| | |
|---|---|
| Tax_Id = 9606 Gene_Symbol =ARHGAP29 Isoform 2 of | IPI00646444 |
| Rho GTPase-activating protein 29 | |

Peptide Information

| | Obsrv. | | | Start | End | |
|---|---|---|---|---|---|---|
| Calc. Mass | Mass | ±da | ±ppm | Seq. | Seq. | Sequence |
| 932.5597 | 932.4811 | −.0786 | −84 | 85 | 92 | VLKSIMNK |
| 950.436 | 950.4933 | .0573 | 60 | 298 | 304 | NEMEKQR |
| 1018.5237 | 1018.5897 | .066 | 65 | 318 | 326 | MLEAENALK |
| 1018.5237 | 1018.5897 | .066 | 65 | 318 | 326 | MLEAENALK |
| 1057.6517 | 1057.5472 | −.1045 | −99 | 289 | 297 | FVQPLLGRK |
| 1057.6517 | 1057.5472 | −.1045 | −99 | 289 | 297 | FVQPLLGRK |
| 1102.6255 | 1102.5885 | −.037 | −34 | 221 | 229 | NIVSWVEKK |
| 1116.5031 | 1116.5959 | .0928 | 83 | 64 | 72 | EAIFSDCFK |

| | |
|---|---|
| Tax_Id = 9606 Gene_Symbol = SETD2 231 kDa protein | IPI00917114 |

Peptide Information

| | Obsrv. | | | Start | End | |
|---|---|---|---|---|---|---|
| Calc. Mass | Mass | ±da | ±ppm | Seq. | Seq. | Sequence |
| 818.4042 | 818.405 | .0008 | 1 | 608 | 613 | HLYEEK |
| 818.4042 | 818.405 | .0008 | 1 | 608 | 613 | HLYEEK |
| 829.4315 | 829.4913 | .0598 | 72 | 953 | 958 | DPQRWK |
| 840.4283 | 840.3886 | −.0397 | −47 | 2017 | 2022 | ELKYCK |
| 877.4639 | 877.437 | −.0269 | −31 | 1538 | 1545 | GRDAVGFR |
| 877.4639 | 877.437 | −.0269 | −31 | 1538 | 1545 | GRDAVGFR |
| 891.4418 | 891.4531 | .0113 | 13 | 18 | 25 | TSENEAIK |
| 954.4778 | 954.4321 | −.0457 | −48 | 1996 | 2003 | ITTTEDFK |
| 980.5047 | 980.4961 | −.0086 | −9 | 885 | 892 | DFSKNLEK |
| 1018.5779 | 1018.5897 | .0118 | 12 | 148 | 156 | ELDSLSKVK |
| 1018.5779 | 1018.5897 | .0118 | 12 | 148 | 156 | ELDSLSKVK |
| 1047.5615 | 1047.557 | −.0045 | −4 | 94 | 102 | GSELRMINK |
| 1091.646 | 1091.5599 | −.0861 | −79 | 853 | 861 | FLLSLQKDK |
| 1102.6508 | 1102.5885 | −.0623 | −57 | 126 | 134 | KLDELPIFK |
| 1103.5844 | 1103.588 | .0036 | 3 | 414 | 423 | SSEFLKHAGK |
| 1753.8909 | 1753.8547 | −.0362 | −21 | 1897 | 1910 | DPEGKIYYYH |
| | | | | | | VITR |
| 1753.9385 | 1753.8547 | −.0838 | −48 | 1137 | 1151 | WTVNGQLRV |
| | | | | | | GFFTTK |

| | |
|---|---|
| Tax_Id = 9606 Gene_Symbol = SETD2 Isoform 2 of | IPI00796144 |
| Histone-lysine N-methyltransferase | |
| SETD2 | |

Peptide Information

| | | | | Start | End | |
|---|---|---|---|---|---|---|
| Calc. Mass | Obsrv. Mass | ±da | ±ppm | Seq. | Seq. | Sequence |
| 818.4042 | 818.405 | .0008 | 1 | 1111 | 1116 | HLYEEK |
| 818.4042 | 818.405 | .0008 | 1 | 1111 | 1116 | HLYEEK |
| 829.4315 | 829.4913 | .0598 | 72 | 1456 | 1461 | DPQRWK |
| 840.421 | 840.3886 | −.0324 | −39 | 415 | 421 | TNLSYSR |
| 891.4418 | 891.4531 | .0113 | 13 | 521 | 528 | TSENEAIK |
| 980.5047 | 980.4961 | −.0086 | −9 | 1388 | 1395 | DFSKNLEK |
| 983.4905 | 983.431 | −.0595 | −60 | 386 | 393 | DTRYVSSR |
| 1018.5779 | 1018.5897 | .0118 | 12 | 651 | 659 | ELDSLSKVK |
| 1018.5779 | 1018.5897 | .0118 | 12 | 651 | 659 | ELDSLSKVK |
| 1032.5837 | 1032.6023 | .0186 | 18 | 3 | 11 | QLQPQPPPK |
| 1042.4225 | 1042.4867 | .0642 | 62 | 425 | 432 | SHYYDSDR |
| 1047.5615 | 1047.557 | −.0045 | −4 | 597 | 605 | GSELRMINK |
| 1056.532 | 1056.5374 | .0054 | 5 | 482 | 490 | DLRTSSYSK |
| 1091.646 | 1091.5599 | −.0861 | −79 | 1356 | 1364 | FLLSLQKDK |
| 1102.6508 | 1102.5885 | −.0623 | −57 | 629 | 637 | KLDELPIFK |
| 1103.5844 | 1103.588 | .0036 | 3 | 917 | 926 | SSEFLKHAGK |
| 1136.5881 | 1136.5674 | −.0207 | −18 | 43 | 53 | GPMFKGVAS |
| | | | | | | SR |
| 1753.9385 | 1753.8547 | −.0838 | −48 | 1640 | 1654 | WTVNGQLRV |
| | | | | | | GFFTTK |
| 1753.9385 | 1753.8547 | −.0838 | −48 | 1640 | 1654 | WTVNGQLRV |
| | | | | | | GFFTTK |

| | |
|---|---|
| Tax_Id = 9606 Gene_Symbol = - Conserved | IPI00017234 |
| hypothetical protein | |

Peptide Information

| | Obsrv. | | | Start | End | |
|---|---|---|---|---|---|---|
| Calc. Mass | Mass | ±da | ±ppm | Seq. | Seq. | Sequence |
| 818.4042 | 818.405 | .0008 | 1 | 92 | 97 | HLYEEK |
| 818.4042 | 818.405 | .0008 | 1 | 92 | 97 | HLYEEK |
| 877.46 | 877.437 | −.023 | −26 | 1 | 7 | MTPLPYR |
| 877.46 | 877.437 | −.023 | −26 | 1 | 7 | MTPLPYR |
| 1120.5931 | 1120.5658 | −.0273 | −24 | 1 | 9 | MTPLPYRSR |
| 1136.5881 | 1136.5674 | −.0207 | −18 | 1 | 9 | MTPLPYRSR |

| | |
|---|---|
| Tax_Id = 9606 Gene_Symbol = HMMR cDNA FLJ53375, | IPI00941941 |
| highly similar to Hyaluronan mediated motility | |
| receptor | |

Peptide Information

| | Obsrv. | | | Start | End | |
|---|---|---|---|---|---|---|
| Calc. Mass | Mass | ±da | ±ppm | Seq. | Seq. | Sequence |
| 818.473 | 818.405 | −.068 | −83 | 630 | 636 | SEVSKLR |
| 818.473 | 818.405 | −.068 | −83 | 630 | 636 | SEVSKLR |
| 830.4366 | 830.4764 | .0398 | 48 | 52 | 58 | QNLNVDK |
| 832.4523 | 832.4264 | −.0259 | −31 | 385 | 391 | SRAEELK |
| 840.3888 | 840.3886 | −.0002 | 0 | 158 | 164 | MRGMMAK |
| 877.4261 | 877.437 | .0109 | 12 | 178 | 185 | SLEESQGK |
| 877.4261 | 877.437 | .0109 | 12 | 178 | 185 | SLEESQGK |
| 923.4217 | 923.4602 | .0385 | 42 | 131 | 138 | FSENGNQK |
| 923.4217 | 923.4602 | .0385 | 42 | 131 | 138 | FSENGNQK |
| 932.5411 | 932.4811 | −.06 | −64 | 123 | 130 | TNELLKSK |
| 946.5641 | 946.4946 | −.0695 | −73 | 142 | 149 | ILSLELMK |
| 1018.5754 | 1018.5897 | .0143 | 14 | 1 | 9 | MSFPKAPLK |
| 1018.5778 | 1018.5897 | .0119 | 12 | 496 | 504 | SALKETEIK |
| 1047.5503 | 1047.557 | .0067 | 6 | 270 | 277 | CQLLEKEK |
| 1064.5986 | 1064.5959 | −.0027 | −3 | 505 | 513 | EITVSFLQK |
| 1089.5535 | 1089.5704 | .0169 | 16 | 378 | 386 | QLEEEAKSR |
| 1089.5535 | 1089.5704 | .0169 | 16 | 378 | 386 | QLEEEAKSR |
| 1103.5327 | 1103.588 | .0553 | 50 | 531 | 539 | QLEDEEG |
| | | | | | | RK |
| 1217.626 | 1217.6008 | −.0252 | −21 | 106 | 115 | IQDLETEL |
| | | | | | | EK |
| 1363.67 | 1363.7206 | .0506 | 37 | 623 | 634 | DENSQLK |
| | | | | | | SEVSK |

| | |
|---|---|
| Tax_Id = 9606 Gene_Symbol = SETD2 | IPI00916993 |
| Uncharacterized protein | |

Peptide Information

| | Obsrv. | | | Start | End | |
|---|---|---|---|---|---|---|
| Calc. Mass | Mass | ±da | ±ppm | Seq. | Seq. | Sequence |
| 818.4042 | 818.405 | .0008 | 1 | 1067 | 1072 | HLYEEK |
| 818.4042 | 818.405 | .0008 | 1 | 1067 | 1072 | HLYEEK |
| 840.421 | 840.3886 | −.0324 | −39 | 371 | 377 | TNLSYSR |
| 891.4418 | 891.4531 | .0113 | 13 | 477 | 484 | TSENEAIK |
| 983.4905 | 983.431 | −.0595 | −60 | 342 | 349 | DTRYVSSR |
| 998.5087 | 998.4193 | −.0894 | −90 | 1 | 9 | MFKGVASSR |
| 1018.5779 | 1018.5897 | .0118 | 12 | 607 | 615 | ELDSLSKVK |
| 1018.5779 | 1018.5897 | .0118 | 12 | 607 | 615 | ELDSLSKVK |
| 1042.4225 | 1042.4867 | .0642 | 62 | 381 | 388 | SHYYDSDR |
| 1047.5615 | 1047.557 | −.0045 | −4 | 553 | 561 | GSELRMINK |
| 1056.532 | 1056.5374 | .0054 | 5 | 438 | 446 | DLRTSSYSK |
| 1091.646 | 1091.5599 | −.0861 | −79 | 1312 | 1320 | FLLSLQKDK |
| 1102.6508 | 1102.5885 | −.0623 | −57 | 585 | 593 | KLDELPIFK |
| 1103.5844 | 1103.588 | .0036 | 3 | 873 | 882 | SSEFLKHAGK |

| | |
|---|---|
| Tax_Id = 9606 Gene_Symbol = CCDC110 Isoform 2 of | IPI00784583 |
| Coiled-coil domain-containing protein | |
| 110 | |

Peptide Information

| | | | | Start | End | |
|---|---|---|---|---|---|---|
| Calc. Mass | Obsrv. Mass | ±da | ±ppm | Seq. | Seq. | Sequence |
| 946.5203 | 946.4946 | −.0257 | −27 | 23 | 31 | ILNSSEGVK |
| 954.4778 | 954.4321 | −.0457 | −48 | 733 | 740 | EYLSLSDK |
| 983.5269 | 983.431 | −.0959 | −98 | 687 | 694 | KHSQENIK |
| 986.4326 | 986.4275 | −.0051 | −5 | 93 | 100 | SHHFEDSK |
| 987.504 | 987.4055 | −.0985 | −100 | 306 | 313 | LSKSEMHR |
| 1002.4727 | 1002.4139 | −.0588 | −59 | 295 | 301 | FHVHFCR |
| 1012.4993 | 1012.4543 | −.045 | −44 | 1 | 8 | MSPEKQHR |
| 1016.5887 | 1016.5704 | −.0183 | −18 | 359 | 366 | ERQPFL |
| | | | | | | VK |
| 1032.6453 | 1032.6023 | −.043 | −42 | 186 | 194 | SKITVPF |
| | | | | | | LK |
| 1033.5347 | 1033.5358 | .0011 | 1 | 210 | 217 | QMKEEL |
| | | | | | | QK |
| 1033.5347 | 1033.5358 | .0011 | 1 | 210 | 217 | QMKEEL |
| | | | | | | QK |
| 1040.4718 | 1040.5715 | .0997 | 96 | 721 | 728 | DTLEFE |
| | | | | | | MR |
| 1056.4667 | 1056.5374 | .0707 | 67 | 721 | 728 | DTLEFE |
| | | | | | | MR |
| 1069.4727 | 1069.4808 | .0081 | 8 | 530 | 538 | MSIEME |
| | | | | | | AMK |
| 1069.4727 | 1069.4808 | .0081 | 8 | 530 | 538 | MSIEME |
| | | | | | | AMK |
| 1085.4675 | 1085.4945 | .027 | 25 | 530 | 538 | MSIEME |
| | | | | | | AMK |
| 1104.5895 | 1104.5812 | −.0083 | −8 | 561 | 570 | EKSSLG |
| | | | | | | NELK |
| 1230.6589 | 1230.7039 | .045 | 37 | 330 | 339 | NITDLKF |
| | | | | | | HSR |

| | |
|---|---|
| Tax_Id = 9606 Gene_Symbol = SETD2 Isoform 1 of | IPI00307733 |
| Histone-lysine N-methyltransferase | |
| SETD2 | |

Peptide Information

| | | | | Start | End | |
|---|---|---|---|---|---|---|
| Calc. Mass | Obsrv. Mass | ±da | ±ppm | Seq. | Seq. | Sequence |
| 818.4042 | 818.405 | .0008 | 1 | 1111 | 1116 | HLYEEK |
| 818.4042 | 818.405 | .0008 | 1 | 1111 | 1116 | HLYEEK |
| 829.4315 | 829.4913 | .0598 | 72 | 1456 | 1461 | DPQRWK |
| 840.421 | 840.3886 | −.0324 | −39 | 415 | 421 | TNLSYSR |
| 877.4639 | 877.437 | −.0269 | −31 | 2041 | 2048 | GRDAVGFR |
| 877.4639 | 877.437 | −.0269 | −31 | 2041 | 2048 | GRDAVGFR |
| 891.4418 | 891.4531 | .0113 | 13 | 521 | 528 | TSENEAIK |
| 954.4778 | 954.4321 | −.0457 | −48 | 2499 | 2506 | ITTTEDFK |
| 980.5047 | 980.4961 | −.0086 | −9 | 1388 | 1395 | DFSKNLEK |
| 983.4905 | 983.431 | −.0595 | −60 | 386 | 393 | DTRYVSSR |
| 1018.5779 | 1018.5897 | .0118 | 12 | 651 | 659 | ELDSLSKVK |
| 1018.5779 | 1018.5897 | .0118 | 12 | 651 | 659 | ELDSLSKVK |
| 1032.5837 | 1032.6023 | .0186 | 18 | 3 | 11 | QLQPQPPPK |
| 1042.4225 | 1042.4867 | .0642 | 62 | 425 | 432 | SHYYDSDR |
| 1047.5615 | 1047.557 | −.0045 | −4 | 597 | 605 | GSELRMINK |
| 1056.532 | 1056.5374 | .0054 | 5 | 482 | 490 | DLRTSSYSK |
| 1091.646 | 1091.5599 | −.0861 | −79 | 1356 | 1364 | FLLSLQKDK |
| 1102.6508 | 1102.5885 | −.0623 | −57 | 629 | 637 | KLDELPIFK |
| 1103.5844 | 1103.588 | .0036 | 3 | 917 | 926 | SSEFLKHAGK |
| 1136.5881 | 1136.5674 | −.0207 | −18 | 43 | 53 | GPMFKGVASSR |
| 1753.8909 | 1753.8547 | −.0362 | −21 | 2400 | 2413 | DPEGKIYYYHVI |
| | | | | | | TR |
| 1753.9385 | 1753.8547 | −.0838 | −48 | 1640 | 1654 | WTVNGQLRVG |
| | | | | | | FFTTK |

| | |
|---|---|
| Tax_Id = 9606 Gene_Symbol = HDAC1 cDNA FLJ60860, | IPI00910637 |
| highly similar to Histone deacetylase 1 | |

Peptide Information

| Calc. Mass | Obsrv. Mass | ±da | ±ppm | Start Seq. | End Seq. | Sequence |
|---|---|---|---|---|---|---|
| 908.4254 | 908.4479 | .0225 | 25 | 1 | 8 | MAQTQGTR |
| 964.4921 | 964.5281 | .036 | 37 | 193 | 200 | VMTVSFHK |
| | 964.5281 | | | | | |
| 1040.4684 | 1040.5715 | .1031 | 99 | 67 | 74 | YHSDDYIK |
| 1064.5266 | 1064.5959 | .0693 | 65 | 1 | 9 | MAQTQGTRR |
| 1089.551 | 1089.5704 | .0194 | 18 | 51 | 58 | MEIYRPHK |
| 1089.551 | 1089.5704 | .0194 | 18 | 51 | 58 | MEIYRPHK |
| 1217.646 | 1217.6008 | −.0452 | −37 | 50 | 58 | KMEIYRPHK |
| 1442.6581 | 1442.7466 | .0885 | 61 | 78 | 89 | SIRPDNMSEYSK |
| 1623.8312 | 1623.7913 | −.0399 | −25 | 37 | 49 | MTHNLLLNYGLYR |

| | |
|---|---|
| Tax_Id = 9606 Gene_Symbol = HMMR Isoform 2 of | IPI00844515 |
| Hyaluronan mediated motility receptor | |

Peptide Information

| Calc. Mass | Obsrv. Mass | ±da | ±ppm | Start Seq. | End Seq. | Sequence |
|---|---|---|---|---|---|---|
| 818.473 | 818.405 | −.068 | −83 | 638 | 644 | SEVSKLR |
| 818.473 | 818.405 | −.068 | −83 | 638 | 644 | SEVSKLR |
| 830.4366 | 830.4764 | .0398 | 48 | 52 | 58 | QNLNVDK |
| 832.4523 | 832.4264 | −.0259 | −31 | 393 | 399 | SRAEELK |
| 840.3888 | 840.3886 | −.0002 | 0 | 166 | 172 | MRGMMAK |
| 877.4261 | 877.437 | .0109 | 12 | 186 | 193 | SLEESQGK |
| 877.4261 | 877.437 | .0109 | 12 | 186 | 193 | SLEESQGK |
| 923.4217 | 923.4602 | .0385 | 42 | 139 | 146 | FSENGNQK |
| 923.4217 | 923.4602 | .0385 | 42 | 139 | 146 | FSENGNQK |
| 932.5411 | 932.4811 | −.06 | −64 | 131 | 138 | TNELLKSK |
| 946.5641 | 946.4946 | −.0695 | −73 | 150 | 157 | ILSLELMK |
| 1018.5754 | 1018.5897 | .0143 | 14 | 1 | 9 | MSFPKAPLK |
| 1018.5778 | 1018.5897 | .0119 | 12 | 504 | 512 | SALKETEIK |
| 1047.5503 | 1047.557 | .0067 | 6 | 278 | 285 | CQLLEKEK |
| 1064.5986 | 1064.5959 | −.0027 | −3 | 513 | 521 | EITVSFLQK |
| 1089.5535 | 1089.5704 | .0169 | 16 | 386 | 394 | QLEEEAKSR |
| 1089.5535 | 1089.5704 | .0169 | 16 | 386 | 394 | QLEEEAKSR |
| 1103.5327 | 1103.588 | .0553 | 50 | 539 | 547 | QLEDEEGRK |
| 1217.626 | 1217.6008 | −.0252 | −21 | 90 | 99 | IQDLETELEK |
| 1363.67 | 1363.7206 | .0506 | 37 | 631 | 642 | DENSQLKSEVSK |

| | |
|---|---|
| Tax_Id = 9606 Gene_Symbol = SETD2 Isoform 3 of | IPI00442150 |
| Histone-lysine N-methyltransferase | |
| SETD2 | |

Peptide Information

| Calc. Mass | Obsrv. Mass | ±da | ±ppm | Start Seq. | End Seq. | Sequence |
|---|---|---|---|---|---|---|
| 818.4042 | 818.405 | .0008 | 1 | 1111 | 1116 | HLYEEK |
| 818.4042 | 818.405 | .0008 | 1 | 1111 | 1116 | HLYEEK |
| 829.4315 | 829.4913 | .0598 | 72 | 1456 | 1461 | DPQRWK |
| 840.421 | 840.3886 | −.0324 | −39 | 415 | 421 | TNLSYSR |
| 891.4418 | 891.4531 | .0113 | 13 | 521 | 528 | TSENEAIK |
| 980.5047 | 980.4961 | −.0086 | −9 | 1388 | 1395 | DFSKNLEK |
| 983.4905 | 983.431 | −.0595 | −60 | 386 | 393 | DTRYVSSR |
| 1018.5779 | 1018.5897 | .0118 | 12 | 651 | 659 | ELDSLSKVK |
| 1018.5779 | 1018.5897 | .0118 | 12 | 651 | 659 | ELDSLSKVK |
| 1032.5837 | 1032.6023 | .0186 | 18 | 3 | 11 | QLQPQPPPK |
| 1042.4225 | 1042.4867 | .0642 | 62 | 425 | 432 | SHYYDSDR |
| 1047.5615 | 1047.557 | −.0045 | −4 | 597 | 605 | GSELRMINK |
| 1056.532 | 1056.5374 | .0054 | 5 | 482 | 490 | DLRTSSYSK |
| 1091.646 | 1091.5599 | −.0861 | −79 | 1356 | 1364 | FLLSLQKDK |
| 1102.6508 | 1102.5885 | −.0623 | −57 | 629 | 637 | KLDELPIFK |
| 1103.5844 | 1103.588 | .0036 | 3 | 917 | 926 | SSEFLKHAGK |
| 1136.5881 | 1136.5674 | −.0207 | −18 | 43 | 53 | GPMFKGVASSR |

| | |
|---|---|
| Tax_Id = 9606 Gene_Symbol = NAV2 Isoform 3 of | IPI00815697 |
| Neuron navigator 2 | |

Peptide Information

| Calc. Mass | Obsrv. Mass | ±da | ±ppm | Start Seq. | End Seq. | Sequence |
|---|---|---|---|---|---|---|
| 818.4479 | 818.405 | −.0429 | −52 | 507 | 513 | RASVTER |
| 818.4479 | 818.405 | −.0429 | −52 | 507 | 513 | RASVTER |
| 877.4122 | 877.437 | .0248 | 28 | 276 | 284 | GGSTTANNR |
| 877.4122 | 877.437 | .0248 | 28 | 276 | 284 | GGSTTANNR |
| 923.4581 | 923.4602 | .0021 | 2 | 813 | 820 | FINTESGR |
| 923.4581 | 923.4602 | .0021 | 2 | 813 | 820 | FINTESGR |
| 946.5568 | 946.4946 | −.0622 | −66 | 41 | 49 | LGSKVEVSK |
| 1012.517 | 1012.4543 | −.0627 | −62 | 192 | 199 | QQQQQPQK |
| 1033.5135 | 1033.5358 | .0223 | 22 | 32 | 40 | AGPQPCYLK |
| 1033.5135 | 1033.5358 | .0223 | 22 | 32 | 40 | AGPQPCYLK |
| 1040.6099 | 1040.5715 | −.0384 | −37 | 1052 | 1062 | TKPSAPAGALK |
| 1057.6365 | 1057.5472 | −.0893 | −84 | 1152 | 1161 | IPKSSALVSR |
| 1057.6438 | 1057.5472 | −.0966 | −91 | 2 | 11 | PAILVASKMK |
| | 1057.5472 | | | | | |
| 1069.5425 | 1069.4808 | −.0617 | −58 | 2325 | 2333 | RAPWEDPAK |
| 1069.5425 | 1069.4808 | −.0617 | −58 | 2325 | 2333 | RAPWEDPAK |
| 1089.5283 | 1089.5704 | .0421 | 39 | 1727 | 1737 | GNGTAQSADLR |
| 1089.5283 | 1089.5704 | .0421 | 39 | 1727 | 1737 | GNGTAQSADLR |
| 1091.5957 | 1091.5599 | −.0358 | −33 | 2317 | 2325 | EGLQLYGRR |
| 1102.48 | 1102.5885 | .1085 | 98 | 286 | 294 | SQSFNNYDK |
| 1103.5691 | 1103.588 | .0189 | 17 | 1690 | 1698 | DSELNELRK |
| 1118.5841 | 1118.5358 | −.0483 | −43 | 1291 | 1300 | YPDVASPTLR |
| 1120.527 | 1120.5658 | .0388 | 35 | 981 | 990 | NSLWSGDDVK |
| 1134.561 | 1134.5808 | .0198 | 17 | 274 | 284 | TRGGSTTANNR |
| 1217.6195 | 1217.6008 | −.0187 | −15 | 1310 | 1320 | QVPIATAENMK |
| 1623.8813 | 1623.7913 | −.09 | −55 | 594 | 608 | LSSGLPQQK |
| | | | | | | PQLDGR |

| | |
|---|---|
| Tax_Id = 9606 Gene_Symbol = NAV2 Isoform 2 of | IPI00816751 |
| Neuron navigator 2 | |

Peptide Information

| Calc. Mass | Obsrv. Mass | ±da | ±ppm | Start Seq. | End Seq. | Sequence |
|---|---|---|---|---|---|---|
| 818.4479 | 818.405 | −.0429 | −52 | 507 | 513 | RASVTER |
| 818.4479 | 818.405 | −.0429 | −52 | 507 | 513 | RASVTER |
| 877.4122 | 877.437 | .0248 | 28 | 276 | 284 | GGSTTANNR |
| 877.4122 | 877.437 | .0248 | 28 | 276 | 284 | GGSTTANNR |
| 923.4581 | 923.4602 | .0021 | 2 | 813 | 820 | FINTESGR |
| 923.4581 | 923.4602 | .0021 | 2 | 813 | 820 | FINTESGR |
| 946.5568 | 946.4946 | −.0622 | −66 | 41 | 49 | LGSKVEVSK |
| 1012.517 | 1012.4543 | −.0627 | −62 | 192 | 199 | QQQQQPQK |
| 1033.5135 | 1033.5358 | .0223 | 22 | 32 | 40 | AGPQPCYLK |
| 1033.5135 | 1033.5358 | .0223 | 22 | 32 | 40 | AGPQPCYLK |
| 1040.6099 | 1040.5715 | −.0384 | −37 | 1052 | 1062 | TKPSAPAGALK |
| 1057.6365 | 1057.5472 | −.0893 | −84 | 1152 | 1161 | IPKSSALVSR |
| 1057.6438 | 1057.5472 | −.0966 | −91 | 2 | 11 | PAILVASKMK |
| 1069.5425 | 1069.4808 | −.0617 | −58 | 2328 | 2336 | RAPWEDPAK |
| 1069.5425 | 1069.4808 | −.0617 | −58 | 2328 | 2336 | RAPWEDPAK |
| 1089.5283 | 1089.5704 | .0421 | 39 | 1727 | 1737 | GNGTAQSADLR |
| 1089.5283 | 1089.5704 | .0421 | 39 | 1727 | 1737 | GNGTAQSADLR |
| 1091.5957 | 1091.5599 | −.0358 | −33 | 2320 | 2328 | EGLQLYGRR |
| 1102.48 | 1102.5885 | .1085 | 98 | 286 | 294 | SQSFNNYDK |
| | 1102.5885 | | | | | |
| 1103.5691 | 1103.588 | .0189 | 17 | 1690 | 1698 | DSELNELRK |
| 1118.5841 | 1118.5358 | −.0483 | −43 | 1291 | 1300 | YPDVASPTLR |
| 1120.527 | 1120.5658 | .0388 | 35 | 981 | 990 | NSLWSGDDVK |
| 1134.561 | 1134.5808 | .0198 | 17 | 274 | 284 | TRGGSTTANNR |
| 1217.6195 | 1217.6008 | −.0187 | −15 | 1310 | 1320 | QVPIATAENMK |
| 1623.8813 | 1623.7913 | −.09 | −55 | 594 | 608 | LSSGLPQQKPQ |
| | | | | | | LDGR |

| | |
|---|---|
| Tax_Id = 9606 Gene_Symbol = TSSK4 Isoform 2 of | IPI00414440 |
| Testis-specific serine/threonine-protein kinase 4 | |

Peptide Information

| Calc. Mass | Obsrv. Mass | ±da | ±ppm | Start Seq. | End Seq. | Sequence |
|---|---|---|---|---|---|---|
| 1016.592 | 1016.5704 | −.0216 | −21 | 279 | 286 | NLILQMLR |
| 1018.535 | 1018.5897 | .0547 | 54 | 148 | 157 | LMPSLSAAGR |
| 1018.535 | 1018.5897 | .0547 | 54 | 148 | 157 | LMPSLSAAGR |
| 1055.5481 | 1055.5176 | −.0305 | −29 | 330 | 338 | QHQSLQITT |
| 1103.6857 | 1103.588 | −.0977 | −89 | 49 | 58 | VMVAVKIISK |

| | |
|---|---|
| Tax_Id = 9606 Gene_Symbol = HMMR Isoform 3 of | IPI00337772 |
| Hyaluronan mediated motility receptor | |

Peptide Information

| Calc. Mass | Obsrv. Mass | ±da | ±ppm | Start Seq. | End Seq. | Sequence |
|---|---|---|---|---|---|---|
| 818.473 | 818.405 | −.068 | −83 | 654 | 660 | SEVSKLR |
| 818.473 | 818.405 | −.068 | −83 | 654 | 660 | SEVSKLR |
| 830.4366 | 830.4764 | .0398 | 48 | 52 | 58 | QNLNVDK |
| 832.4523 | 832.4264 | −.0259 | −31 | 409 | 415 | SRAEELK |
| 840.3888 | 840.3886 | −.0002 | 0 | 182 | 188 | MRGMMAK |
| 877.4261 | 877.437 | .0109 | 12 | 202 | 209 | SLEESQGK |
| 877.4261 | 877.437 | .0109 | 12 | 202 | 209 | SLEESQGK |
| 923.4217 | 923.4602 | .0385 | 42 | 155 | 162 | FSENGNQK |
| 923.4217 | 923.4602 | .0385 | 42 | 155 | 162 | FSENGNQK |
| 932.5411 | 932.4811 | −.06 | −64 | 147 | 154 | TNELLKSK |
| 946.5641 | 946.4946 | −.0695 | −73 | 166 | 173 | ILSLELMK |
| 1018.5754 | 1018.5897 | .0143 | 14 | 1 | 9 | MSFPKAPLK |
| 1018.5778 | 1018.5897 | .0119 | 12 | 520 | 528 | SALKETEIK |
| 1047.5503 | 1047.557 | .0067 | 6 | 294 | 301 | CQLLEKEK |
| 1064.5986 | 1064.5959 | −.0027 | −3 | 529 | 537 | EITVSFLQK |
| 1089.5535 | 1089.5704 | .0169 | 16 | 402 | 410 | QLEEEAKSR |
| 1089.5535 | 1089.5704 | .0169 | 16 | 402 | 410 | QLEEEAKSR |
| 1103.5327 | 1103.588 | .0553 | 50 | 555 | 563 | QLEDEEG |
| | | | | | | RK |
| 1217.626 | 1217.6008 | −.0252 | −21 | 106 | 115 | IQDLETEL |
| | | | | | | EK |
| 1363.67 | 1363.7206 | .0506 | 37 | 647 | 658 | DENSQLK |
| | | | | | | SEVSK |

4no matched protein found

| | | | | | | |
|---|---|---|---|---|---|---|
| 891.4 | 891.45 | 0.0344 | 3 | 7 | 84 | ESEDQKR |
| 982.4 | 982.43 | −0.0336 | — | 1 | 9 | MGGTTST |

### 6—AFOD

4 new found proteins from fraction 42 which are named KH24 (SEQ ID NO. KH24), KH25 (SEQ ID NO. KH25), KH26 (SEQ ID NO. KH26) and KH27 (SEQ ID NO. KH27) and their functions are under investigation.

| | | | |
|---|---|---|---|
| | 155/G6 | | Instr./Gel Origin |
| | [1] Sample Project | | Instrument |
| | 20111201 | | Sample |
| | | | Name |
| Protein Name | | Accession No. | |

| | |
|---|---|
| Tax_Id = 9606 Gene_Symbol = | IPI00893693 |
| CCDC88A 137 kDa protein | |

KH24 (SEQ ID NO. KH24) Peptide Information

| | Obsrv. | | | Start | End | |
|---|---|---|---|---|---|---|
| Calc. Mass | Mass | ±da | ±ppm | Seq. | Seq. | Sequence |
| 879.4968 | 879.4153 | −0.0815 | −93 | 635 | 642 | KSSMVALK |
| 880.5138 | 880.4396 | −0.0742 | −84 | 129 | 135 | YKLLESK |
| 896.4584 | 896.4399 | −0.0185 | −21 | 365 | 371 | NLEVEHR |
| 985.5789 | 985.582 | 0.0031 | 3 | 16 | 23 | LRQQAEIK |
| 985.5789 | 985.582 | 0.0031 | 3 | 16 | 23 | LRQQAEIK |
| 1021.5272 | 1021.5333 | 0.0061 | 6 | 961 | 969 | ESSLSRQSK |
| 1021.5425 | 1021.5333 | −0.0092 | −9 | 178 | 185 | NYEALKQR |
| 1187.6267 | 1187.6656 | 0.0389 | 33 | 383 | 392 | QKGQLEDLEK |
| | 1187.6656 | | | | | |
| 1187.6267 | 1187.6656 | 0.0389 | 33 | 383 | 392 | QKGQLEDLEK |
| 1199.5903 | 1199.6674 | 0.0771 | 64 | 435 | 444 | ETEVLQTDHK |
| 1254.6212 | 1254.6615 | 0.0403 | 32 | 345 | 355 | QASEYESLISK |
| 1406.7274 | 1406.6833 | −0.0441 | −31 | 372 | 382 | DLEDRYNQLLK |
| 1406.7274 | 1406.6833 | −0.0441 | −31 | 372 | 382 | DLEDRYNQLLK |
| 1428.6754 | 1428.7153 | 0.0399 | 28 | 909 | 921 | SVSGKTPGDFYDR |
| 1479.6996 | 1479.7794 | 0.0798 | 54 | 875 | 887 | SSSQENLLDEVMK |
| 1502.8425 | 1502.8582 | 0.0157 | 10 | 78 | 90 | TLVTLREDLVSEK |
| 1727.9286 | 1727.8947 | −0.0339 | −20 | 223 | 237 | LIEVERNNATLQAEK |
| 2213.1084 | 2213.2441 | 0.1357 | 61 | 935 | 955 | KTEDTYFISS |
| | | | | | | AGKPTPGT |
| | | | | | | QGK |
| 2233.0918 | 2233.0076 | −0.0842 | −38 | 515 | 532 | TLLEQNMES |
| | | | | | | KDLFHVEQ R |
| 2233.0918 | 2233.2017 | 0.1099 | 49 | 515 | 532 | TLLEQNMESKDLFHVEQ |

IPI01012199

Protein Group

IPI00256861
R
Tax_Id = 9606 Gene_Symbol = MACF1
Uncharacterized protein
Tax_Id = 9606 Gene_Symbol = MACF1 Isoform 2 of
Microtubule-actin cross-linking factor 1, isoforms
1/2/3/5

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3736 | 3743 | LMALGPIR |
| 870.5229 | 870.5385 | 0.0156 | 18 | 3736 | 3743 | LMALGPIR |
| 879.4935 | 879.4153 | −0.0782 | −89 | 1874 | 1881 | FVTISGQK |
| 880.441 | 880.4396 | −0.0014 | −2 | 2240 | 2246 | DFTELQK |
| 910.4265 | 910.4448 | 0.0183 | 20 | 3476 | 3482 | YSEIQDR |
| 910.4265 | 910.4448 | 0.0183 | 20 | 3476 | 3482 | YSEIQDR |
| 928.4669 | 928.4629 | −0.004 | −4 | 3652 | 3658 | KEVMEHR |
| 1021.5499 | 1021.5333 | −0.0166 | −16 | 2551 | 2558 | QQVQFMLK |
| 1021.5499 | 1021.5333 | −0.0166 | −16 | 2551 | 2558 | QQVQFMLK |
| 1106.5034 | 1106.5583 | 0.0549 | 50 | 1018 | 1026 | LEEEVEACK |
| 1170.6841 | 1170.6443 | −0.0398 | −34 | 3312 | 3321 | VVKAQIQEQK |
| 1187.6201 | 1187.6656 | 0.0455 | 38 | 2757 | 2766 | NCPISAKLER |
| 1187.6201 | 1187.6656 | 0.0455 | 38 | 2757 | 2766 | NCPISAKLER |
| 1199.6896 | 1199.6674 | −0.0222 | −19 | 3758 | 3767 | AFSIDIIRHK |
| 1257.6797 | 1257.6525 | −0.0272 | −22 | 1506 | 1516 | QISEQLNALNK |
| 1257.6797 | 1257.6525 | −0.0272 | −22 | 1506 | 1516 | QISEQLNALNK |
| 1261.694 | 1261.6499 | −0.0441 | −35 | 380 | 389 | LLEVWIEFGR |
| 1287.6791 | 1287.6593 | −0.0198 | −15 | 4662 | 4672 | EKTLLPEDSQK |
| 1320.7271 | 1320.6016 | −0.1255 | −95 | 1870 | 1881 | GDLRFVTISGQK |
| 1406.7386 | 1406.6833 | −0.0553 | −39 | 4647 | 4658 | QPVYDTTIRTGR |
| 1406.7386 | 1406.6833 | −0.0553 | −39 | 4647 | 4658 | QPVYDTTIRTGR |
| 1413.7809 | 1413.8057 | 0.0248 | 18 | 3156 | 3167 | ARQEQLELTLGR |
| 1420.7213 | 1420.6881 | −0.0332 | −23 | 2940 | 2951 | TGSLEEMTQRLR |
| 1425.7156 | 1425.8075 | 0.0919 | 64 | 869 | 880 | NTISVKAVCDYR |
| 1428.7693 | 1428.7153 | −0.054 | −38 | 5052 | 5063 | LNDALDRLEELK |
| 1465.7281 | 1465.7726 | 0.0445 | 30 | 4428 | 4439 | EETYNQLLDKGR |
| 1465.7316 | 1465.7726 | 0.041 | 28 | 4440 | 4453 | LMLLSRDDSGS |
| | | | | | | GSK |
| 1487.7952 | 1487.7654 | −0.0298 | −20 | 3565 | 3577 | QTTGEEVLLIQ |
| | | | | | | EK |
| 1502.873 | 1502.8582 | −0.0148 | −10 | 380 | 391 | LLEVWIEFGRIK |
| 1532.6785 | 1532.7728 | 0.0943 | 62 | 3891 | 3903 | ELNPEEGEMV |
| | | | | | | EEK |
| 1713.8728 | 1713.8539 | −0.0189 | −11 | 3123 | 3137 | HMLEEEGTLDL |
| | | | | | | LGLK |
| 1727.9149 | 1727.8947 | −0.0202 | −12 | 2151 | 2165 | KLLPQAEMFEH |
| | | | | | | LSGK |
| 1794.9636 | 1794.8103 | −0.1533 | −85 | 5106 | 5121 | QEFIDGILASKF |
| | | | | | | PTTK |
| 1838.8412 | 1838.927 | 0.0858 | 47 | 4960 | 4974 | ALIAEHQTFME |
| | | | | | | EMTR |
| 2186.155 | 2185.9851 | −0.1699 | −78 | 1958 | 1978 | LLSDTVASDPG |
| | | | | | | VLQEQL A |
| | | | | | | TTK |
| 2202.1799 | 2201.9719 | −0.208 | −94 | 2864 | 2882 | MSELRVTLDPV |
| | | | | | | QLESSLLR |
| 2233.1135 | 2233.0076 | −0.1059 | −47 | 2462 | 2481 | EALAGLLVTYP |
| | | | | | | NSQEAE N |
| | | | | | | WK |
| 2233.1135 | 2233.2017 | 0.0882 | 39 | 2462 | 2481 | EALAGLLVTYP |
| | | | | | | NSQEAE N |
| | | | | | | WK |
| 2299.0217 | 2299.144 | 0.1223 | 53 | 3068 | 3088 | EMFSQLADLD |
| | | | | | | DELDGM |

IPI00982053

Peptide Information

Tax_Id = 9606 Gene_Symbol = TSSK6 Conserved hypothetical protein
G AIGR

| | | | | Start | End | |
|---|---|---|---|---|---|---|
| Calc. Mass | Obsrv. Mass | ±da | ±ppm | Seq. | Seq. | Sequence |
| 856.4999 | 856.5223 | 0.0224 | 26 | 89 | 97 | AAQIAGAVR |
| 879.4907 | 879.4153 | −0.0754 | −86 | 55 | 62 | ATPAHRAR |
| 880.3876 | 880.4396 | 0.052 | 59 | 267 | 273 | GNMRSCR |
| 896.3825 | 896.4399 | 0.0574 | 64 | 267 | 273 | GNMRSCR |
| 912.4574 | 912.4597 | 0.0023 | 3 | 125 | 132 | LTDFGFGR |
| 1187.6136 | 1187.6656 | 0.052 | 44 | 271 | 279 | SCRVLLHMR |
| 1187.6136 | 1187.6656 | 0.052 | 44 | 271 | 279 | SCRVLLHMR |
| 1332.6768 | 1332.6146 | −0.0622 | −47 | 26 | 40 | GHQGGGPAAS |
| | | | | | | APGLR |
| 1413.771 | 1413.8057 | 0.0347 | 25 | 148 | 160 | GAPGHPLRPQ |
| | | | | | | EVR |
| 1487.7272 | 1487.7654 | 0.0382 | 26 | 111 | 122 | CENVLLSPDERR |
| 2299.2095 | 2299.144 | −0.0655 | −28 | 240 | 260 | LEAGWFQPFL |
| | | | | | | QPRALGQ |
| | | | | | | GGAR |

IPI01018834
IPI00478226

Protein Group

Tax_Id = 9606 Gene_Symbol = MACF1
Uncharacterized protein
Tax_Id = 9606 Gene_Symbol = MACF1 Isoform 5 of
Microtubule-actin cross-linking factor 1, isoforms
1/2/3/5

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3606 | 3613 | LMALGPIR |
| 870.5229 | 870.5385 | 0.0156 | 18 | 3606 | 3613 | LMALGPIR |
| 879.4935 | 879.4153 | −0.0782 | −89 | 1874 | 1881 | FVTISGQK |
| 880.441 | 880.4396 | −0.0014 | −2 | 2240 | 2246 | DFTELQK |
| 928.4669 | 928.4629 | −0.004 | −4 | 3522 | 3528 | KEVMEHR |
| 1021.5499 | 1021.5333 | −0.0166 | −16 | 2530 | 2537 | QQVQFMLK |
| 1021.5499 | 1021.5333 | −0.0166 | −16 | 2530 | 2537 | QQVQFMLK |
| 1106.5034 | 1106.5583 | 0.0549 | 50 | 1018 | 1026 | LEEEVEACK |
| 1170.6841 | 1170.6443 | −0.0398 | −34 | 3291 | 3300 | VVKAQIQEQK |
| 1187.6201 | 1187.6656 | 0.0455 | 38 | 2736 | 2745 | NCPISAKLER |
| 1187.6201 | 1187.6656 | 0.0455 | 38 | 2736 | 2745 | NCPISAKLER |
| 1199.6896 | 1199.6674 | −0.0222 | −19 | 3628 | 3637 | AFSIDIIRHK |
| 1257.6797 | 1257.6525 | −0.0272 | −22 | 1506 | 1516 | QISEQLNALNK |
| 1257.6797 | 1257.6525 | −0.0272 | −22 | 1506 | 1516 | QISEQLNA |
| | | | | | | LNK |
| 1261.694 | 1261.6499 | −0.0441 | −35 | 380 | 389 | LLEVWIEF |
| | | | | | | GR |
| 1287.6791 | 1287.6593 | −0.0198 | −15 | 4532 | 4542 | EKTLLPED |
| | | | | | | SQK |
| 1320.7271 | 1320.6016 | −0.1255 | −95 | 1870 | 1881 | GDLRFVTI |
| | | | | | | SGQK |
| 1406.7386 | 1406.6833 | −0.0553 | −39 | 4517 | 4528 | QPVYDTTI |
| | | | | | | RTGR |
| 1406.7386 | 1406.6833 | −0.0553 | −39 | 4517 | 4528 | QPVYDTTI |
| | | | | | | RTGR |
| 1413.7809 | 1413.8057 | 0.0248 | 18 | 3135 | 3146 | ARQEQLE |
| | | | | | | LTLGR |
| 1420.7213 | 1420.6881 | −0.0332 | −23 | 2919 | 2930 | TGSLEEMTQRLR |
| 1425.7156 | 1425.8075 | 0.0919 | 64 | 869 | 880 | NTISVKAVCDYR |
| 1428.7693 | 1428.7153 | −0.054 | −38 | 4922 | 4933 | LNDALDRLEELK |
| 1465.7281 | 1465.7726 | 0.0445 | 30 | 4298 | 4309 | EETYNQLLDKGR |
| 1465.7316 | 1465.7726 | 0.041 | 28 | 4310 | 4323 | LMLLSRDDSGS |
| | | | | | | GSK |
| 1487.7952 | 1487.7654 | −0.0298 | −20 | 3435 | 3447 | QTTGEEVLLIQ |
| | | | | | | EK |
| 1502.873 | 1502.8582 | −0.0148 | −10 | 380 | 391 | LLEVWIEFGRIK |
| 1532.6785 | 1532.7728 | 0.0943 | 62 | 3761 | 3773 | ELNPEEGEMV |
| | | | | | | EEK |
| 1713.8728 | 1713.8539 | −0.0189 | −11 | 3102 | 3116 | HMLEEEGTLDL |
| | | | | | | LGLK |
| 1727.9149 | 1727.8947 | −0.0202 | −12 | 2151 | 2165 | KLLPQAEMFEH |
| | | | | | | LSGK |
| 1794.9636 | 1794.8103 | −0.1533 | −85 | 4976 | 4991 | QEFIDGILASKF |
| | | | | | | PTTK |
| 1838.8412 | 1838.927 | 0.0858 | 47 | 4830 | 4844 | ALIAEHQTFME |
| | | | | | | EMTR |
| 2186.155 | 2185.9851 | −0.1699 | −78 | 1958 | 1978 | LLSDTVASDPG |
| | | | | | | VLQEQL A |
| | | | | | | TTK |
| 2202.1799 | 2201.9719 | −0.208 | −94 | 2843 | 2861 | MSELRVTLDPV |
| | | | | | | QLESSLLR |
| 2233.1135 | 2233.0076 | −0.1059 | −47 | 2441 | 2460 | EALAGLLVTYP |
| | | | | | | NSQEAE N |
| | | | | | | WK |
| 2233.1135 | 2233.2017 | 0.0882 | 39 | 2441 | 2460 | EALAGLLVTYP |
| | | | | | | NSQEAE N |
| | | | | | | WK |
| 2299.0217 | 2299.144 | 0.1223 | 53 | 3047 | 3067 | EMFSQLADLDD |
| | | | | | | ELDGM G |
| | | | | | | AIGR |

IPI01018940

Peptide Information

Tax_Id = 9606 Gene_Symbol = MACF1 Isoform 3 of
Microtubule-actin cross-linking factor 1, isoforms
1/2/3/5

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3680 | 3687 | LMALGPIR |
| 870.5229 | 870.5385 | 0.0156 | 18 | 3680 | 3687 | LMALGPIR |
| 879.4935 | 879.4153 | −0.0782 | −89 | 1839 | 1846 | FVTISGQK |
| 880.441 | 880.4396 | −0.0014 | −2 | 2205 | 2211 | DFTELQK |
| 910.4265 | 910.4448 | 0.0183 | 20 | 3420 | 3426 | YSEIQDR |
| 910.4265 | 910.4448 | 0.0183 | 20 | 3420 | 3426 | YSEIQDR |
| 928.4669 | 928.4629 | −0.004 | −4 | 3596 | 3602 | KEVMEHR |
| 1021.5499 | 1021.5333 | −0.0166 | −16 | 2495 | 2502 | QQVQFMLK |
| 1021.5499 | 1021.5333 | −0.0166 | −16 | 2495 | 2502 | QQVQFM |
| | | | | | | LK |
| 1106.5034 | 1106.5583 | 0.0549 | 50 | 983 | 991 | LEEEVE |
| | | | | | | ACK |
| 1170.6841 | 1170.6443 | −0.0398 | −34 | 3256 | 3265 | VVKAQI |
| | | | | | | QEQK |
| 1187.6201 | 1187.6656 | 0.0455 | 38 | 2701 | 2710 | NCPISAK |
| | | | | | | LER |
| 1187.6201 | 1187.6656 | 0.0455 | 38 | 2701 | 2710 | NCPISAKLER |
| 1199.6896 | 1199.6674 | −0.0222 | −19 | 3702 | 3711 | AFSIDIIRHK |
| 1257.6797 | 1257.6525 | −0.0272 | −22 | 1471 | 1481 | QISEQLNALNK |
| 1257.6797 | 1257.6525 | −0.0272 | −22 | 1471 | 1481 | QISEQLNALNK |
| 1261.694 | 1261.6499 | −0.0441 | −35 | 345 | 354 | LLEVWIEFGR |
| 1287.6791 | 1287.6593 | −0.0198 | −15 | 4606 | 4616 | EKTLLPEDSQK |
| 1320.7271 | 1320.6016 | −0.1255 | −95 | 1835 | 1846 | GDLRFVTISGQK |
| 1406.7386 | 1406.6833 | −0.0553 | −39 | 4591 | 4602 | QPVYDTTIRTGR |
| 1406.7386 | 1406.6833 | −0.0553 | −39 | 4591 | 4602 | QPVYDTTIRTGR |
| 1413.7809 | 1413.8057 | 0.0248 | 18 | 3100 | 3111 | ARQEQLELTLGR |
| 1420.7213 | 1420.6881 | −0.0332 | −23 | 2884 | 2895 | TGSLEEMTQRLR |
| 1425.7156 | 1425.8075 | 0.0919 | 64 | 834 | 845 | NTISVKAVCDYR |
| 1428.7693 | 1428.7153 | −0.054 | −38 | 4996 | 5007 | LNDALDRLEELK |
| 1465.7281 | 1465.7726 | 0.0445 | 30 | 4372 | 4383 | EETYNQLLDKGR |
| 1465.7316 | 1465.7726 | 0.041 | 28 | 4384 | 4397 | LMLLSRDDSGS |
| | | | | | | GSK |
| 1487.7952 | 1487.7654 | −0.0298 | −20 | 3509 | 3521 | QTTGEEVLLIQ |
| | | | | | | EK |
| 1502.873 | 1502.8582 | −0.0148 | −10 | 345 | 356 | LLEVWIEFGRIK |
| 1532.6785 | 1532.7728 | 0.0943 | 62 | 3835 | 3847 | ELNPEEGEMV |
| | | | | | | EEK |
| 1713.8728 | 1713.8539 | −0.0189 | −11 | 3067 | 3081 | HMLEEEGTLDL |
| | | | | | | LGLK |
| 1727.9149 | 1727.8947 | −0.0202 | −12 | 2116 | 2130 | KLLPQAEMFEH |
| | | | | | | LSGK |
| 1794.9636 | 1794.8103 | −0.1533 | −85 | 5050 | 5065 | QEFIDGILASKF |
| | | | | | | PTTK |
| 1838.8412 | 1838.927 | 0.0858 | 47 | 4904 | 4918 | ALIAEHQTFME |
| | | | | | | EMTR |
| 2186.155 | 2185.9851 | −0.1699 | −78 | 1923 | 1943 | LLSDTVASDPG |
| | | | | | | VLQEQL A |
| | | | | | | TTK |
| 2202.1799 | 2201.9719 | −0.208 | −94 | 2808 | 2826 | MSELRVTLDPV |
| | | | | | | QLESSLLR |
| 2233.1135 | 2233.0076 | −0.1059 | −47 | 2406 | 2425 | EALAGLLVTYP |
| | | | | | | NSQEAE N |
| | | | | | | WK |
| 2233.1135 | 2233.2017 | 0.0882 | 39 | 2406 | 2425 | EALAGLLVTYP |
| | | | | | | NSQEAE N |
| | | | | | | WK |
| 2299.0217 | 2299.144 | 0.1223 | 53 | 3012 | 3032 | EMFSQLADLDD |
| | | | | | | ELDGM G |
| | | | | | | AIGR |

IPI00152653

Peptide Information

Tax_Id = 9606 Gene_Symbol = DNAH5 Dynein heavy
chain 5, axonemal

| | | | | Start | End | |
|---|---|---|---|---|---|---|
| Calc. Mass | Obsrv. Mass | ±da | ±ppm | Seq. | Seq. | Sequence |
| 856.525 | 856.5223 | −0.0027 | −3 | 1408 | 1414 | QLNLLQK |
| 879.4683 | 879.4153 | −0.053 | −60 | 1654 | 1660 | RFSNI |
| | | | | | | DK |
| 880.4774 | 880.4396 | −0.0378 | −43 | 1204 | 1211 | FALTAETK |
| 896.4407 | 896.4399 | −0.0008 | −1 | 747 | 753 | RNFSNMK |
| 910.488 | 910.4448 | −0.0432 | −47 | 1702 | 1709 | SLTGYLEK |
| 910.488 | 910.4448 | −0.0432 | −47 | 1702 | 1709 | SLTGYLEK |
| 912.4573 | 912.4597 | 0.0024 | 3 | 285 | 291 | AELEHWK |
| 928.5403 | 928.4629 | −0.0774 | −83 | 4440 | 4446 | IPAWWKK |
| 985.5941 | 985.582 | −0.0121 | −12 | 2503 | 2509 | RLELWLR |
| 985.5941 | 985.582 | −0.0121 | −12 | 2503 | 2509 | RLELWLR |
| 1005.5363 | 1005.6074 | 0.0711 | 71 | 820 | 827 | VNDLIEFR |
| 1021.4805 | 1021.5333 | 0.0528 | 52 | 2103 | 2111 | SVAMMVPDR |
| 1021.4805 | 1021.5333 | 0.0528 | 52 | 2103 | 2111 | SVAMMVPDR |
| 1106.5411 | 1106.5583 | 0.0172 | 16 | 326 | 333 | TWREMDIR |
| 1187.6816 | 1187.6656 | −0.016 | −13 | 4549 | 4558 | NMKLIESKPK |
| 1187.6816 | 1187.6656 | −0.016 | −13 | 4549 | 4558 | NMKLIESKPK |
| 1199.6995 | 1199.6674 | −0.0321 | −27 | 2585 | 2596 | AVLLIGEQGTAK |
| 1257.7566 | 1257.6525 | −0.1041 | −83 | 167 | 177 | LLSDIFIPALR |
| 1257.7566 | 1257.6525 | −0.1041 | −83 | 167 | 177 | LLSDIFIPALR |
| 1261.6212 | 1261.6499 | 0.0287 | 23 | 1299 | 1308 | VDTLHYAWEK |
| 1271.6553 | 1271.6659 | 0.0106 | 8 | 3711 | 3721 | TSIIDFTVTMK |
| 1332.7369 | 1332.6146 | −0.1223 | −92 | 3210 | 3222 | LKEASESVAAL |
| | | | | | | SK |
| 1413.8577 | 1413.8057 | −0.052 | −37 | 166 | 177 | RLLSDIFIPALR |
| 1428.7482 | 1428.7153 | −0.0329 | −23 | 3698 | 3710 | LPNPAYTPEIS |
| | | | | | | AR |
| 1502.9153 | 1502.8582 | −0.0571 | −38 | 1119 | 1132 | LVSVLSTIINST |
| | | | | | | KK |
| 1794.7972 | 1794.8103 | 0.0131 | 7 | 748 | 761 | NFSNMKMMLA |
| | | | | | | EYQR |
| 1838.8668 | 1838.927 | 0.0602 | 33 | 3501 | 3515 | ERWTEQSQEF |
| | | | | | | AAQTK |
| 2266.176 | 2266.0767 | −0.0993 | −44 | 957 | 975 | ELLSHFNHQN |
| | | | | | | MDALLKV TR |

IPI00966721

Peptide Information

Tax_Id = 9606 Gene_Symbol = C5orf28
Uncharacterized protein

| | Obsrv. | | | Start | End | |
|---|---|---|---|---|---|---|
| Calc. Mass | Mass | ±da | ±ppm | Seq. | Seq. | Sequence |
| 870.5043 | 870.5385 | 0.0342 | 39 | 2 | 9 | EAALTLPR |
| 870.5043 | 870.5385 | 0.0342 | 39 | 2 | 9 | EAALTLPR |

IPI00853050
Tax_Id = 9606 Gene_Symbol = MB21D1
Uncharacterized protein

Peptide Information

| | | | | Start | End | |
|---|---|---|---|---|---|---|
| Calc. Mass | Obsrv. Mass | ±da | ±ppm | Seq. | Seq. | Sequence |
| 924.4897 | 924.4626 | −0.0271 | −29 | 385 | 392 | EILNNHGK |
| 985.5425 | 985.582 | 0.0395 | 40 | 157 | 166 | DAAPGASKLR |
| 985.5425 | 985.582 | 0.0395 | 40 | 157 | 166 | DAAPGASKLR |
| 1021.6153 | 1021.5333 | −0.082 | −80 | 188 | 196 | GVVDHLLLR |
| 1021.6153 | 1021.5333 | −0.082 | −80 | 188 | 196 | GVVDHLLLR |
| 1254.6161 | 1254.6615 | 0.0454 | 36 | 2 | 11 | QPWHGKAMQR |
| 1257.5422 | 1257.6525 | 0.1103 | 88 | 496 | 505 | NNEFPVFDEF |
| 1257.5422 | 1257.6525 | 0.1103 | 88 | 496 | 505 | NNEFPVFDEF |
| 1271.7206 | 1271.6659 | −0.0547 | −43 | 303 | 315 | GGSPAVTLLISEK |
| 1287.6652 | 1287.6593 | −0.0059 | −5 | 12 | 25 | ASEAGATAPKA |
| | | | | | | SAR |
| 1413.6719 | 1413.8057 | 0.1338 | 95 | 220 | 231 | ISAPNEFDVMFK |
| 1479.7472 | 1479.7794 | 0.0322 | 22 | 174 | 187 | LSRDDISTAAG |
| | | | | | | MVK |

IPI00854821

Peptide Information

Tax_Id = 9606 Gene_Symbol = DLG5 Isoform 4 of
Disks
large homolog 5

| | | | | Start | End | |
|---|---|---|---|---|---|---|
| Calc. Mass | Obsrv. Mass | ±da | ±ppm | Seq. | Seq. | Sequence |
| 879.3876 | 879.4153 | 0.0277 | 31 | 132 | 138 | DDVDMLR |
| 880.4523 | 880.4396 | −0.0127 | −14 | 206 | 212 | DYDALRK |
| 924.4421 | 924.4626 | 0.0205 | 22 | 1766 | 1772 | LEQEYSR |
| 985.5537 | 985.582 | 0.0283 | 29 | 139 | 146 | RENGQLLR |
| 985.5537 | 985.582 | 0.0283 | 29 | 139 | 146 | RENGQLLR |
| 1187.5917 | 1187.6656 | 0.0739 | 62 | 712 | 722 | AHGPEVQAHNK |
| 1187.5917 | 1187.6656 | 0.0739 | 62 | 712 | 722 | AHGPEVQAHNK |
| 1261.6205 | 1261.6499 | 0.0294 | 23 | 358 | 368 | KAANEEMEALR |
| 1406.7526 | 1406.6833 | −0.0693 | −49 | 1495 | 1506 | LADVEQELSFKK |
| 1406.7526 | 1406.6833 | −0.0693 | −49 | 1495 | 1506 | LADVEQELSFKK |
| 1420.7026 | 1420.6881 | −0.0145 | −10 | 1562 | 1575 | DDNSATKTLSA |
| | | | | | | AAR |
| 1487.8315 | 1487.7654 | −0.0661 | −44 | 339 | 351 | LQTEVELAESKLK |
| 1502.7632 | 1502.8582 | 0.095 | 63 | 359 | 371 | AANEEMEALRQ |
| | | | | | | IK |
| 1727.9539 | 1727.8947 | −0.0592 | −34 | 1243 | 1259 | VQKGSEPLGISI |
| | | | | | | VSGEK |

IPI00927275

Peptide Information

Tax_Id = 9606 Gene_Symbol = LMCD1
Uncharacterized protein

| Calc. | Obsrv. | | | Start | End | |
|---|---|---|---|---|---|---|
| Mass | Mass | ±da | ±ppm | Seq. | Seq. | Sequence |
| 870.5043 | 870.5385 | 0.0342 | 39 | 7 | 14 | DLNPGVKK |
| 870.5043 | 870.5385 | 0.0342 | 39 | 7 | 14 | DLNPGVKK |
| 1106.5623 | 1106.5583 | −0.004 | −4 | 15 | 24 | MSLGQ |
| | | | | | | LQSAR |
| 1420.6063 | 1420.6881 | 0.0818 | 58 | 33 | 44 | GTCSGF |
| | | | | | | EPHSWR |
| 2265.9951 | 2266.0767 | 0.0816 | 36 | 25 | 44 | GVACLGC |
| | | | | | | KGTC |
| | | | | | | SGFEPH |

5no matched protein found

| 156/G7 | Instr./Gel Origin |
|---|---|
| [1] Sample Project | |
| 20111201 | Instrument Sample |

| | | |
|---|---|---|
| Accession No. | Protein Name | Name |

| | |
|---|---|
| IPI00160622 | Tax_Id = 9606 Gene_Symbol = |
| | CEP250 Isoform 1 of |
| | Centrosome-associated protein |
| | CEP250 |

KH25 (SEQ ID NO. 25) Peptide Information

| | | | | Start | End | |
|---|---|---|---|---|---|---|
| Calc. Mass | Obsrv. Mass | ±da | ±ppm | Seq. | Seq. | Sequence |
| 985.5537 | 985.5696 | 0.0159 | 16 | 399 | 406 | RQAVQDLR |
| 985.5789 | 985.5696 | −0.0093 | −9 | 127 | 135 | ADWNKALR |
| 1065.5067 | 1065.5122 | 0.0055 | 5 | 883 | 890 | EKMELEMR |
| 1232.6117 | 1232.6262 | 0.0145 | 12 | 1390 | 1399 | LKNEEVESER |
| 1235.5837 | 1235.5809 | −0.0028 | −2 | 68 | 76 | SWCQELEKR |
| 1257.691 | 1257.6628 | −0.0282 | −22 | 1667 | 1676 | IQVLEDQRTR |
| 1257.705 | 1257.6628 | −0.0422 | −34 | 601 | 612 | LSALNEALALDK |
| 1323.728 | 1323.6946 | −0.0334 | −25 | 172 | 182 | GEHGRLLSLWR |
| 1425.7081 | 1425.8451 | 0.137 | 96 | 2371 | 2382 | QDYITRSAQTSR |
| 1425.7081 | 1425.8451 | 0.137 | 96 | 2371 | 2382 | QDYITRSAQTSR |
| 1487.77 | 1487.8041 | 0.0341 | 23 | 753 | 766 | QDLAEQLQGLSS |
| | | | | | | AK |
| 1497.8384 | 1497.7552 | −0.0832 | −56 | 1881 | 1893 | RVQALEEVLGDLR |
| 1532.785 | 1532.8186 | 0.0336 | 22 | 1698 | 1709 | ELTTQRQLMQER |
| 1579.7819 | 1579.8885 | 0.1066 | 67 | 522 | 534 | ERLQEMLMGLEAK |
| 1708.9089 | 1708.9078 | −0.0011 | −1 | 2292 | 2305 | HNVQLRSTLEQV |
| | | | | | | ER |
| | 1708.9078 | | | | | |
| 1713.8767 | 1713.9175 | 0.0408 | 24 | 492 | 507 | VNVELQLQGDSA |
| | | | | | | QGQK |

| | |
|---|---|
| IPI00292871 | Tax_Id = 9606 Gene_Symbol = CEP250 Isoform 2 of |
| | Centrosome-associated protein CEP250 |

Peptide Information

| | Obsrv. | | | Start | End | |
|---|---|---|---|---|---|---|
| Calc. Mass | Mass | ±da | ±ppm | Seq. | Seq. | Sequence |
| 985.5537 | 985.5696 | 0.0159 | 16 | 399 | 406 | RQAVQDLR |
| 985.5789 | 985.5696 | −0.0093 | −9 | 127 | 135 | ADWNKALR |
| 1232.6117 | 1232.6262 | 0.0145 | 12 | 1334 | 1343 | LKNEEVESER |
| 1235.5837 | 1235.5809 | −0.0028 | −2 | 68 | 76 | SWCQELEKR |
| 1257.691 | 1257.6628 | −0.0282 | −22 | 1611 | 1620 | IQVLEDQRTR |
| 1257.705 | 1257.6628 | −0.0422 | −34 | 601 | 612 | LSALNEALALDK |
| 1323.728 | 1323.6946 | −0.0334 | −25 | 172 | 182 | GEHGRLLSLWR |
| 1425.7081 | 1425.8451 | 0.137 | 96 | 2315 | 2326 | QDYITRSAQTSR |
| 1425.7081 | 1425.8451 | 0.137 | 96 | 2315 | 2326 | QDYITRSAQTSR |
| 1487.77 | 1487.8041 | 0.0341 | 23 | 753 | 766 | QDLAEQLQGL |
| | | | | | | SSAK |
| 1497.8384 | 1497.7552 | −0.0832 | −56 | 1825 | 1837 | RVQALEEVLG |
| | | | | | | DLR |
| 1532.785 | 1532.8186 | 0.0336 | 22 | 1642 | 1653 | ELTTQRQLMQ |
| | | | | | | ER |
| 1579.7819 | 1579.8885 | 0.1066 | 67 | 522 | 534 | ERLQEMLMGL |
| | | | | | | EAK |
| 1708.9089 | 1708.9078 | −0.0011 | −1 | 2236 | 2249 | HNVQLRSTLE |
| | | | | | | QVER |
| 1713.8767 | 1713.9175 | 0.0408 | 24 | 492 | 507 | VNVELQLQGD |
| | | | | | | SAQGQK |

| | |
|---|---|
| IPI00945119 | Tax_Id = 9606 Gene_Symbol = CEP250 |
| | Uncharacterized protein |

Peptide Information

| | | | | Start | End | |
|---|---|---|---|---|---|---|
| Calc. Mass | Obsrv. Mass | ±da | ±ppm | Seq. | Seq. | Sequence |
| 985.5537 | 985.5696 | 0.0159 | 16 | 399 | 406 | RQAVQDLR |
| 985.5789 | 985.5696 | −0.0093 | −9 | 127 | 135 | ADVVNKALR |
| 1235.5837 | 1235.5809 | −0.0028 | −2 | 68 | 76 | SWCQELEKR |
| 1323.728 | 1323.6946 | −0.0334 | −25 | 172 | 182 | GEHGRLLSLWR |
| 1713.8767 | 1713.9175 | 0.0408 | 24 | 492 | 507 | VNVELQLQGDSA |
| | | | | | | QGQK |

| | |
|---|---|
| IPI01012199 | Tax_Id = 9606 Gene_Symbol = MACF1 Uncharacterized |
| | protein |

Protein Group

| | |
|---|---|
| IPI00256861 | Tax_Id = 9606 Gene_Symbol = MACF1 Isoform 2 of |
| | Microtubule-actin cross-linking factor 1, isoforms 1/2/3/5 |

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5196 | −0.0033 | −4 | 3736 | 3743 | LMALGPIR |
| 880.441 | 880.4194 | −0.0216 | −25 | 2240 | 2246 | DFTELQK |
| 910.4265 | 910.4317 | 0.0052 | 6 | 3476 | 3482 | YSEIQDR |
| 910.4265 | 910.4317 | 0.0052 | 6 | 3476 | 3482 | YSEIQDR |
| 928.4669 | 928.4491 | −0.0178 | −19 | 3652 | 3658 | KEVMEHR |
| 1021.5499 | 1021.5277 | −0.0222 | −22 | 2551 | 2558 | QQVQFMLK |
| 1021.5499 | 1021.5277 | −0.0222 | −22 | 2551 | 2558 | QQVQFMLK |
| 1170.5902 | 1170.6512 | 0.061 | 52 | 2820 | 2828 | NHWEELSKK |
| 1170.6841 | 1170.6512 | −0.0329 | −28 | 3312 | 3321 | WKAQIQEQK |
| 1187.6201 | 1187.681 | 0.0609 | 51 | 2757 | 2766 | NCPISAKLER |
| 1187.6201 | 1187.681 | 0.0609 | 51 | 2757 | 2766 | NCPISAKLER |
| 1232.6117 | 1232.6262 | 0.0145 | 12 | 2659 | 2668 | QQLEETSEIR |
| 1235.6378 | 1235.5809 | −0.0569 | −46 | 1058 | 1066 | LRLEEYEQR |
| 1257.6797 | 1257.6628 | −0.0169 | −13 | 1506 | 1516 | QISEQLNALNK |
| 1257.6797 | 1257.6628 | −0.0169 | −13 | 1506 | 1516 | QISEQLNALNK |
| 1261.694 | 1261.6696 | −0.0244 | −19 | 380 | 389 | LLEVWIEFGR |
| 1320.7271 | 1320.6184 | −0.1087 | −82 | 1870 | 1881 | GDLRFVTISGQK |
| 1323.7896 | 1323.6946 | −0.095 | −72 | 3670 | 3680 | ALLELVPWR |
| | | | | | | AR |
| 1406.7386 | 1406.7107 | −0.0279 | −20 | 4647 | 4658 | QPVYDTTIR |
| | | | | | | TGR |
| 1406.7386 | 1406.7107 | −0.0279 | −20 | 4647 | 4658 | QPVYDTTIR |
| | | | | | | TGR |
| 1413.7809 | 1413.8478 | 0.0669 | 47 | 3156 | 3167 | ARQEQLELT |
| | | | | | | LGR |
| 1420.7213 | 1420.7368 | 0.0155 | 11 | 2940 | 2951 | TGSLEEMTQ |
| | | | | | | RLR |
| 1425.7156 | 1425.8451 | 0.1295 | 91 | 869 | 880 | NTISVKAVC |
| | | | | | | DYR |
| 1425.7156 | 1425.8451 | 0.1295 | 91 | 869 | 880 | NTISVKAVC |
| | | | | | | DYR |
| 1428.7693 | 1428.7944 | 0.0251 | 18 | 5052 | 5063 | LNDALDRLE |
| | | | | | | ELK |
| 1465.7281 | 1465.8011 | 0.073 | 50 | 4428 | 4439 | EETYNQLLD |
| | | | | | | KGR |
| 1465.7316 | 1465.8011 | 0.0695 | 47 | 4440 | 4453 | LMLLSRDDS |
| | | | | | | GSGSK |
| 1487.7952 | 1487.8041 | 0.0089 | 6 | 3565 | 3577 | QTTGEEVLLI |
| | | | | | | QEK |
| 1502.873 | 1502.8989 | 0.0259 | 17 | 380 | 391 | LLEVWIEFG |
| | | | | | | RIK |
| 1532.6785 | 1532.8186 | 0.1401 | 91 | 3891 | 3903 | ELNPEEGE |
| | | | | | | MVEEK |
| 1708.8389 | 1708.9078 | 0.0689 | 40 | 3681 | 3695 | EGLDKLVSDAN |
| | | | | | | EQYK |
| 1713.8728 | 1713.9175 | 0.0447 | 26 | 3123 | 3137 | HMLEEEGTLDL |
| | | | | | | LGLK |
| 1727.9149 | 1727.9177 | 0.0028 | 2 | 2151 | 2165 | KLLPQAEMFEH |
| | | | | | | LSGK |
| 1950.9412 | 1951.001 | 0.0598 | 31 | 4960 | 4975 | ALIAEHQTFME |
| | | | | | | EMTRK |
| 1966.9362 | 1966.9954 | 0.0592 | 30 | 4960 | 4975 | ALIAEHQTFME |
| | | | | | | EMTRK |
| 2185.0693 | 2185.1575 | 0.0882 | 40 | 3885 | 3903 | IGPQLKELNPE |
| | | | | | | EGEMVEEK |
| 2186.155 | 2186.0022 | −0.1528 | −70 | 1958 | 1978 | LLSDTVASDPG |
| | | | | | | VLQEQLA |
| | | | | | | TTK |
| 2186.1851 | 2186.1931 | 0.008 | 4 | 2864 | 2882 | MSELRVTLDPV |
| | | | | | | QLESSLLR |
| 2200.0632 | 2200.0994 | 0.0362 | 16 | 4015 | 4031 | EIQDKLDQMVF |
| | | | | | | FWEDIK |
| 2202.1799 | 2202.2275 | 0.0476 | 22 | 2864 | 2882 | MSELRVTLDPV |
| | | | | | | QLESSLLR |
| 2212.2183 | 2212.3137 | 0.0954 | 43 | 3558 | 3577 | NGQALLKQTTG |
| | | | | | | EEVLLIQ |
| | | | | | | EK |

| | |
|---|---|
| IPI01018940 | Tax_Id = 9606 Gene_Symbol = MACF1 Isoform 3 of |
| | Microtubule-actin cross-linking factor 1, |
| | isoforms 1/2/3/5 |

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5196 | −0.0033 | −4 | 3680 | 3687 | LMALGPIR |
| 880.441 | 880.4194 | −0.0216 | −25 | 2205 | 2211 | DFTELQK |
| 910.4265 | 910.4317 | 0.0052 | 6 | 3420 | 3426 | YSEIQDR |
| 910.4265 | 910.4317 | 0.0052 | 6 | 3420 | 3426 | YSEIQDR |
| 928.4669 | 928.4491 | −0.0178 | −19 | 3596 | 3602 | KEVMEHR |
| 1021.5499 | 1021.5277 | −0.0222 | −22 | 2495 | 2502 | QQVQFMLK |
| 1021.5499 | 1021.5277 | −0.0222 | −22 | 2495 | 2502 | QQVQFMLK |
| 1170.5902 | 1170.6512 | 0.061 | 52 | 2764 | 2772 | NHWEELSKK |
| 1170.6841 | 1170.6512 | −0.0329 | −28 | 3256 | 3265 | VVKAQIQEQK |
| 1187.6201 | 1187.681 | 0.0609 | 51 | 2701 | 2710 | NCPISAKL |
| | | | | | | ER |
| 1187.6201 | 1187.681 | 0.0609 | 51 | 2701 | 2710 | NCPISAKL |
| | | | | | | ER |
| 1232.6117 | 1232.6262 | 0.0145 | 12 | 2603 | 2612 | QQLEETS |
| | | | | | | EIR |
| 1235.6378 | 1235.5809 | −0.0569 | −46 | 1023 | 1031 | LRLEEYE |
| | | | | | | QR |
| 1257.6797 | 1257.6628 | −0.0169 | −13 | 1471 | 1481 | QISEQLNA |
| | | | | | | LNK |
| 1257.6797 | 1257.6628 | −0.0169 | −13 | 1471 | 1481 | QISEQLNA |
| | | | | | | LNK |
| 1261.694 | 1261.6696 | −0.0244 | −19 | 345 | 354 | LLEVWIEF |
| | | | | | | GR |
| 1320.7271 | 1320.6184 | −0.1087 | −82 | 1835 | 1846 | GDLRFVTI |
| | | | | | | SGQK |
| 1323.7896 | 1323.6946 | −0.095 | −72 | 3614 | 3624 | ALLELVP |
| | | | | | | WRAR |
| 1406.7386 | 1406.7107 | −0.0279 | −20 | 4591 | 4602 | QPVYDTTIRTGR |
| 1406.7386 | 1406.7107 | −0.0279 | −20 | 4591 | 4602 | QPVYDTTIRTGR |
| 1413.7809 | 1413.8478 | 0.0669 | 47 | 3100 | 3111 | ARQEQLELTLGR |
| 1420.7213 | 1420.7368 | 0.0155 | 11 | 2884 | 2895 | TGSLEEMTQRLR |
| 1425.7156 | 1425.8451 | 0.1295 | 91 | 834 | 845 | NTISVKAVCDYR |
| 1425.7156 | 1425.8451 | 0.1295 | 91 | 834 | 845 | NTISVKAVCDYR |
| 1428.7693 | 1428.7944 | 0.0251 | 18 | 4996 | 5007 | LNDALDRLEELK |
| 1465.7281 | 1465.8011 | 0.073 | 50 | 4372 | 4383 | EETYNQLLDKGR |
| 1465.7316 | 1465.8011 | 0.0695 | 47 | 4384 | 4397 | LMLLSRDDSGS |
| | | | | | | GSK |
| 1487.7952 | 1487.8041 | 0.0089 | 6 | 3509 | 3521 | QTTGEEVLLIQ |
| | | | | | | EK |
| 1502.873 | 1502.8989 | 0.0259 | 17 | 345 | 356 | LLEVWIEFGRIK |
| 1532.6785 | 1532.8186 | 0.1401 | 91 | 3835 | 3847 | ELNPEEGEMVE |
| | | | | | | EK |
| 1708.8389 | 1708.9078 | 0.0689 | 40 | 3625 | 3639 | EGLDKLVSDAN |
| | | | | | | EQYK |
| 1713.8728 | 1713.9175 | 0.0447 | 26 | 3067 | 3081 | HMLEEEGTLDL |
| | | | | | | LGLK |
| 1727.9149 | 1727.9177 | 0.0028 | 2 | 2116 | 2130 | KLLPQAEMFEH |
| | | | | | | LSGK |
| 1950.9412 | 1951.001 | 0.0598 | 31 | 4904 | 4919 | ALIAEHQTFME |
| | | | | | | EMTRK |
| 1966.9362 | 1966.9954 | 0.0592 | 30 | 4904 | 4919 | ALIAEHQTFME |
| | | | | | | EMTRK |
| 2185.0693 | 2185.1575 | 0.0882 | 40 | 3829 | 3847 | IGPQLKELNPE |
| | | | | | | EGEMVEEK |
| 2186.155 | 2186.0022 | −0.1528 | −70 | 1923 | 1943 | LLSDTVASDPG |
| | | | | | | VLQEQLA |
| | | | | | | TTK |
| 2186.1851 | 2186.1931 | 0.008 | 4 | 2808 | 2826 | MSELRVTLDPV |
| | | | | | | QLESSLLR |
| 2200.0632 | 2200.0994 | 0.0362 | 16 | 3959 | 3975 | EIQDKLDQMVF |
| | | | | | | FWEDIK |
| 2202.1799 | 2202.2275 | 0.0476 | 22 | 2808 | 2826 | MSELRVTLDPV |
| | | | | | | QLESSLLR |
| 2212.2183 | 2212.3137 | 0.0954 | 43 | 3502 | 3521 | NGQALLKQTTG |
| | | | | | | EEVLLIQ |
| | | | | | | EK |

| | |
|---|---|
| IPI01018834 | Tax_Id = 9606 Gene_Symbol = MACF1 Uncharacterized |
| | protein |

Protein Group

| | |
|---|---|
| IPI00478226 | Tax_Id = 9606 Gene_Symbol = MACF1 Isoform 5 of |
| | Microtubule-actin cross-linking factor 1, isoforms 1/2/3/5 |

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5196 | −0.0033 | −4 | 3606 | 3613 | LMALGPIR |
| 880.441 | 880.4194 | −0.0216 | −25 | 2240 | 2246 | DFTELQK |
| 928.4669 | 928.4491 | −0.0178 | −19 | 3522 | 3528 | KEVMEHR |
| 1021.5499 | 1021.5277 | −0.0222 | −22 | 2530 | 2537 | QQVQFMLK |
| 1021.5499 | 1021.5277 | −0.0222 | −22 | 2530 | 2537 | QQVQFMLK |
| 1170.5902 | 1170.6512 | 0.061 | 52 | 2799 | 2807 | NHWEELSKK |
| 1170.6841 | 1170.6512 | −0.0329 | −28 | 3291 | 3300 | VVKAQIQEQK |
| 1187.6201 | 1187.681 | 0.0609 | 51 | 2736 | 2745 | NCPISAKLER |
| 1187.6201 | 1187.681 | 0.0609 | 51 | 2736 | 2745 | NCPISAKLER |
| 1232.6117 | 1232.6262 | 0.0145 | 12 | 2638 | 2647 | QQLEETSEIR |
| 1235.6378 | 1235.5809 | −0.0569 | −46 | 1058 | 1066 | LRLEEYEQR |
| 1257.6797 | 1257.6628 | −0.0169 | −13 | 1506 | 1516 | QISEQLNALNK |
| 1257.6797 | 1257.6628 | −0.0169 | −13 | 1506 | 1516 | QISEQLNALNK |
| 1261.694 | 1261.6696 | −0.0244 | −19 | 380 | 389 | LLEVWIEFGR |
| 1320.7271 | 1320.6184 | −0.1087 | −82 | 1870 | 1881 | GDLRFVTISGQK |
| 1323.7896 | 1323.6946 | −0.095 | −72 | 3540 | 3550 | ALLELVPWRAR |
| 1406.7386 | 1406.7107 | −0.0279 | −20 | 4517 | 4528 | QPVYDTTIRTGR |
| 1406.7386 | 1406.7107 | −0.0279 | −20 | 4517 | 4528 | QPVYDTTIRTGR |
| 1413.7809 | 1413.8478 | 0.0669 | 47 | 3135 | 3146 | ARQEQLELTLGR |
| 1420.7213 | 1420.7368 | 0.0155 | 11 | 2919 | 2930 | TGSLEEMTQRLR |
| 1425.7156 | 1425.8451 | 0.1295 | 91 | 869 | 880 | NTISVKAVCDYR |
| 1425.7156 | 1425.8451 | 0.1295 | 91 | 869 | 880 | NTISVKAVCDYR |
| 1428.7693 | 1428.7944 | 0.0251 | 18 | 4922 | 4933 | LNDALDRLEELK |
| 1465.7281 | 1465.8011 | 0.073 | 50 | 4298 | 4309 | EETYNQLLDKGR |
| 1465.7316 | 1465.8011 | 0.0695 | 47 | 4310 | 4323 | LMLLSRDDSGS |
| | | | | | | GSK |
| 1487.7952 | 1487.8041 | 0.0089 | 6 | 3435 | 3447 | QTTGEEVLLIQ |
| | | | | | | EK |
| 1502.873 | 1502.8989 | 0.0259 | 17 | 380 | 391 | LLEVWIEFGRIK |
| 1532.6785 | 1532.8186 | 0.1401 | 91 | 3761 | 3773 | ELNPEEGEMVE |
| | | | | | | EK |
| 1708.8389 | 1708.9078 | 0.0689 | 40 | 3551 | 3565 | EGLDKLVSDAN |
| | | | | | | EQYK |
| 1713.8728 | 1713.9175 | 0.0447 | 26 | 3102 | 3116 | HMLEEEGTLDL |
| | | | | | | LGLK |
| 1727.9149 | 1727.9177 | 0.0028 | 2 | 2151 | 2165 | KLLPQAEMFEH |
| | | | | | | LSGK |
| 1950.9412 | 1951.001 | 0.0598 | 31 | 4830 | 4845 | ALIAEHQTFME |
| | | | | | | EMTRK |
| 1966.9362 | 1966.9954 | 0.0592 | 30 | 4830 | 4845 | ALIAEHQTFME |
| | | | | | | EMTRK |
| 2185.0693 | 2185.1575 | 0.0882 | 40 | 3755 | 3773 | IGPQLKELNPE |
| | | | | | | EGEMVEEK |
| 2186.155 | 2186.0022 | −0.1528 | −70 | 1958 | 1978 | LLSDTVASDPG |
| | | | | | | VLQEQLA |
| | | | | | | TTK |
| 2186.1851 | 2186.1931 | 0.008 | 4 | 2843 | 2861 | MSELRVTLDPV |
| | | | | | | QLESSLLR |
| 2200.0632 | 2200.0994 | 0.0362 | 16 | 3885 | 3901 | EIQDKLDQMVF |
| | | | | | | FWEDIK |
| 2202.1799 | 2202.2275 | 0.0476 | 22 | 2843 | 2861 | MSELRVTLDPVQLESSLLR |
| | 2202.2275 | | | | | |
| 2212.2183 | 2212.3137 | 0.0954 | 43 | 3428 | 3447 | NGQALLKQTTGEEVLLIQ |
| | | | | | | EK |

| | |
|---|---|
| IPI00853061 | Tax_Id = 9606 |
| | Gene_Symbol = COL6A3 |
| | collagen alpha-3(VI) chain |
| | isoform 2 precursor |

Peptide Information

| | | | | Start | End | |
|---|---|---|---|---|---|---|
| Calc. Mass | Obsrv. Mass | ±da | ±ppm | Seq. | Seq. | Sequence |
| 896.4625 | 896.4256 | −0.0369 | −41 | 246 | 252 | TNFPYVR |
| 910.5104 | 910.4317 | −0.0787 | −86 | 293 | 300 | SDILGHLR |
| 910.5104 | 910.4317 | −0.0787 | −86 | 293 | 300 | SDILGHLR |
| 1187.7008 | 1187.681 | −0.0198 | −17 | 915 | 924 | NIFKRPLGSR |
| 1187.7008 | 1187.681 | −0.0198 | −17 | 915 | 924 | NIFKRPLGSR |
| 1320.7311 | 1320.6184 | −0.1127 | −85 | 558 | 569 | QSGWPFIFQAK |
| 1420.7948 | 1420.7368 | −0.058 | −41 | 635 | 646 | SGFPLLKEFVQR |
| 1425.7445 | 1425.8451 | 0.1006 | 71 | 219 | 231 | TLSGTPEVHSNKR |
| 1425.7445 | 1425.8451 | 0.1006 | 71 | 219 | 231 | TLSGTPEVHSNKR |
| 1579.9781 | 1579.8885 | −0.0896 | −57 | 138 | 153 | AAEGIPKLLVLITG |
| | | | | | | GK |
| 1950.9518 | 1951.001 | 0.0492 | 25 | 1018 | 1036 | YPPPGEMGASEV |
| | | | | | | LLGAF |
| | | | | | | SI |
| 2185.1323 | 2185.1575 | 0.0252 | 12 | 107 | 126 | KMKPLDGSALYT |
| | | | | | | GSALD |
| | | | | | | FVR |
| 2200.2449 | 2200.0994 | −0.1455 | −66 | 524 | 544 | SAGSRIEDGVLQ |
| | | | | | | FLVLLV |
| | | | | | | AGR |
| 2202.1919 | 2202.2275 | 0.0356 | 16 | 549 | 569 | VDGPASNLKQSG |
| | | | | | | VVPFIF |
| | | | | | | QAK |

| | |
|---|---|
| IPI00029760 | Tax_Id = 9606 Gene _Symbol = FGF4 |
| | Fibroblast growth factor 4 |

Peptide Information

| | Obsrv. | | | Start | End | |
|---|---|---|---|---|---|---|
| Calc. Mass | Mass | ±da | ±ppm | Seq. | Seq. | Sequence |
| 1005.5146 | 1005.6021 | 0.0875 | 87 | 190 | 198 | GNRVSPTMK |
| 1257.6686 | 1257.6628 | −0.0058 | −5 | 113 | 123 | DSLLELSPVER |
| 1257.6686 | 1257.6628 | −0.0058 | −5 | 113 | 123 | DSLLELSPVER |
| 1425.7559 | 1425.8451 | 0.0892 | 63 | 174 | 186 | YPGMFIALSKNGK |
| 1425.7559 | 1425.8451 | 0.0892 | 63 | 174 | 186 | YPGMFIALSKNGK |
| 2186.1289 | 2186.0022 | −0.1267 | −58 | 85 | 103 | RLYCNVGIGFHL |
| | | | | | | QALPDGR |
| 2186.1289 | 2186.1931 | 0.0642 | 29 | 85 | 103 | RLYCNVGIGFHL |
| | | | | | | QALPDGR |

| | |
|---|---|
| IPI00291868 | Tax_Id = 9606 Gene_Symbol = FBXO41 |
| | F-box only protein 41 |

Peptide Information

| | | | | Start | End | |
|---|---|---|---|---|---|---|
| Calc. Mass | Obsrv. Mass | ±da | ±ppm | Seq. | Seq. | Sequence |
| 982.4914 | 982.4271 | −0.0643 | −65 | 907 | 914 | LFEDMVTK |
| 1465.7329 | 1465.8011 | 0.0682 | 47 | 30 | 43 | MAGASPAVPHER |
| | | | | | | AR |
| 1465.7329 | 1465.8011 | 0.0682 | 47 | 30 | 43 | MAGASPAVPHER |
| | | | | | | AR |
| 1579.8513 | 1579.8885 | 0.0372 | 24 | 907 | 919 | LFEDMVTKLQALR |
| 1713.9065 | 1713.9175 | 0.011 | 6 | 808 | 826 | ALGVGGAGCGV |
| | | | | | | QGLASL |
| | | | | | | AR |
| 2894.479 | 2894.4836 | 0.0046 | 2 | 2 | 27 | TTGLSDQQVVCD |
| | | | | | | LDHRA |
| | | | | | | VEALLQAVR |

| | |
|---|---|
| IP101012037 | Tax_Id = 9606 Gene_Symbol = MCM8 |
| | Uncharacterized protein |

Peptide Information

| | | | | Start | End | |
|---|---|---|---|---|---|---|
| Calc. Mass | Obsrv. Mass | ±da | ±ppm | Seq. | Seq. | Sequence |
| 982.4523 | 982.4271 | −0.0252 | −26 | 1 | 8 | MNGEYRGR |
| 1065.5225 | 1065.5122 | −0.0103 | −10 | 23 | 32 | GGGNFSGKWR |
| 1479.769 | 1479.8186 | 0.0496 | 34 | 48 | 60 | TSEQTPQFLLSTK |
| 2170.1238 | 2170.114 | −0.0098 | −5 | 127 | 146 | ELTEGGEVTNLIP |
| | | | | | | DIATELR |
| 2185.1467 | 2185.1575 | 0.0108 | 5 | 152 | 170 | TLACMGLAIHQVL |
| | | | | | | TKDLER |
| 2212.1465 | 2212.3137 | 0.1672 | 76 | 147 | 166 | DAPEKTLACMGL |
| | | | | | | AIHQVL |

6no matched protein found

| 157/G8 | Instr./Gel Origin |
|---|---|
| [1] Sample Project | |
| 20111201 | Instrument Sample |

| | | |
|---|---|---|
| Accession No. | Protein Name | Name |

| | |
|---|---|
| IPI00160622 | Tax_Id = 9606 Gene_Symbol = |
| | CEP250 Isoform 1 of |
| | Centrosome-associated protein |
| | CEP250 |

KH26 (SEQ ID NO. KH26) Peptide Information

| | Obsrv. | | | Start | | |
|---|---|---|---|---|---|---|
| Calc. Mass | Mass | ±da | ±ppm | Seq. | End Seq. | Sequence |
| 985.5537 | 985.5631 | 0.0094 | 10 | 399 | 406 | RQAVQDLR |
| 985.5537 | 985.5631 | 0.0094 | 10 | 399 | 406 | RQAVQDLR |
| 1097.4966 | 1097.5127 | 0.0161 | 15 | 883 | 890 | EKMELEMR |
| 1232.6117 | 1232.6179 | 0.0062 | 5 | 1390 | 1399 | LKNEEVESER |
| 1257.691 | 1257.6606 | −0.0304 | −24 | 1667 | 1676 | IQVLEDQRTR |
| 1257.705 | 1257.6606 | −0.0444 | −35 | 601 | 612 | LSALNEALALDK |
| 1283.6776 | 1283.6473 | −0.0303 | −24 | 122 | 132 | LHMEKADVVNK |
| 1350.6471 | 1350.7144 | 0.0673 | 50 | 190 | 200 | HFLEMKSATDR |
| 1425.7081 | 1425.8483 | 0.1402 | 98 | 2371 | 2382 | QDYITRSAQTSR |
| 1425.7081 | 1425.8483 | 0.1402 | 98 | 2371 | 2382 | QDYITRSAQTSR |
| 1487.77 | 1487.7893 | 0.0193 | 13 | 753 | 766 | QDLAEQLQGLS |
| | | | | | | SAK |
| 1532.785 | 1532.8113 | 0.0263 | 17 | 1698 | 1709 | ELTTQRQLMQ |
| | | | | | | ER |
| 1579.7819 | 1579.8809 | 0.099 | 63 | 522 | 534 | ERLQEMLMGL |
| | | | | | | EAK |
| 1657.9484 | 1657.8533 | −0.0951 | −57 | 926 | 939 | ERVSLLETLLQ |
| | | | | | | TQK |
| 1708.9089 | 1708.9053 | −0.0036 | −2 | 2292 | 2305 | HNVQLRSTLEQ |
| | | | | | | VER |
| 1713.8767 | 1713.9238 | 0.0471 | 27 | 492 | 507 | VNVELQLQGD |
| | | | | | | SAQGQK |
| 2092.1001 | 2091.998 | −0.1021 | −49 | 212 | 230 | LSGSLLTCCLR |
| | | | | | | LTVGAQS R |

| | |
|---|---|
| IPI00292871 | Tax_Id = 9606 Gene_Symbol = CEP250 |
| | Isoform 2 of Centrosome-associated protein CEP250 |

Peptide Information

| | Obsrv. | | | Start | End | |
|---|---|---|---|---|---|---|
| Calc. Mass | Mass | ±da | ±ppm | Seq. | Seq. | Sequence |
| 985.5537 | 985.5631 | 0.0094 | 10 | 399 | 406 | RQAVQDLR |
| 985.5537 | 985.5631 | 0.0094 | 10 | 399 | 406 | RQAVQDLR |
| 1232.6117 | 1232.6179 | 0.0062 | 5 | 1334 | 1343 | LKNEEVESER |
| 1257.691 | 1257.6606 | −0.0304 | −24 | 1611 | 1620 | IQVLEDQRTR |
| 1257.705 | 1257.6606 | −0.0444 | −35 | 601 | 612 | LSALNEALALDK |
| 1283.6776 | 1283.6473 | −0.0303 | −24 | 122 | 132 | LHMEKADVVNK |
| 1350.6471 | 1350.7144 | 0.0673 | 50 | 190 | 200 | HFLEMKSATDR |
| 1425.7081 | 1425.8483 | 0.1402 | 98 | 2315 | 2326 | QDYITRSAQTSR |
| 1425.7081 | 1425.8483 | 0.1402 | 98 | 2315 | 2326 | QDYITRSAQTSR |
| 1487.77 | 1487.7893 | 0.0193 | 13 | 753 | 766 | QDLAEQLQGLSSAK |
| 1532.785 | 1532.8113 | 0.0263 | 17 | 1642 | 1653 | ELTTQRQLMQER |
| 1579.7819 | 1579.8809 | 0.099 | 63 | 522 | 534 | ERLQEMLMGLEAK |
| 1657.9484 | 1657.8533 | −0.0951 | −57 | 870 | 883 | ERVSLLETLLQTQK |
| 1708.9089 | 1708.9053 | −0.0036 | −2 | 2236 | 2249 | HNVQLRSTLEQVER |
| | 1708.9053 | | | | | |
| 1713.8767 | 1713.9238 | 0.0471 | 27 | 492 | 507 | VNVELQLQGDSAQGQK |
| 2092.1001 | 2091.998 | −0.1021 | −49 | 212 | 230 | LSGSLLTCCLRLTVGAQSR |

| | |
|---|---|
| IPI00945119 | Tax_Id = 9606 |
| | Gene_Symbol = CEP250 |
| | Uncharacterized protein |

Peptide Information

| | | | | | | |
|---|---|---|---|---|---|---|
| | Obsrv. | | | Start | End | |
| Calc. Mass | Mass | ±da | ±ppm | Seq. | Seq. | Sequence |

| | | | | | | |
|---|---|---|---|---|---|---|
| 985.5537 | 985.5631 | 0.0094 | 10 | 399 | 406 | RQAVQDLR |
| 985.5537 | 985.5631 | 0.0094 | 10 | 399 | 406 | RQAVQDLR |
| 1283.6776 | 1283.6473 | −0.0303 | −24 | 122 | 132 | LHMEKADVVNK |
| 1350.6471 | 1350.7144 | 0.0673 | 50 | 190 | 200 | HFLEMKSATDR |
| 1546.837 | 1546.7799 | −0.0571 | −37 | 524 | 536 | LQSSQLQSCRVLK |
| 1713.8767 | 1713.9238 | 0.0471 | 27 | 492 | 507 | VNVELQLQGDSA |
| | | | | | | QGQK |
| 2092.1001 | 2091.998 | −0.1021 | −49 | 212 | 230 | LSGSLLTCCLRLT |
| | | | | | | VGAQSR |

| | |
|---|---|
| IPI01018940 | Tax_Id = 9606 Gene_Symbol = MACF1 |
| | Isoform 3 of Microtubule-actin cross-linking |
| | factor 1, isoforms 1/2/3/5 |

Peptide Information

| | | | | Start | End | |
|---|---|---|---|---|---|---|
| Calc. Mass | Obsrv. Mass | ±da | ±ppm | Seq. | Seq. | Sequence |
| 842.4519 | 842.4806 | 0.0287 | 34 | 4409 | 4415 | WHVVSSK |
| 870.5229 | 870.511 | −0.0119 | −14 | 3680 | 3687 | LMALGPIR |
| 910.4265 | 910.4239 | −0.0026 | −3 | 3420 | 3426 | YSEIQDR |
| 910.4265 | 910.4239 | −0.0026 | −3 | 3420 | 3426 | YSEIQDR |
| 1021.5499 | 1021.5206 | −0.0293 | −29 | 2495 | 2502 | QQVQFMLK |
| 1021.5499 | 1021.5206 | −0.0293 | −29 | 2495 | 2502 | QQVQFMLK |
| 1170.6841 | 1170.6456 | −0.0385 | −33 | 3256 | 3265 | VVKAQIQEQK |
| 1187.6201 | 1187.673 | 0.0529 | 45 | 2701 | 2710 | NCPISAKLER |
| 1232.6117 | 1232.6179 | 0.0062 | 5 | 2603 | 2612 | QQLEETSEIR |
| 1257.6797 | 1257.6606 | −0.0191 | −15 | 1471 | 1481 | QISEQLNALNK |
| 1257.6797 | 1257.6606 | −0.0191 | −15 | 1471 | 1481 | QISEQLNALNK |
| 1261.694 | 1261.6659 | −0.0281 | −22 | 345 | 354 | LLEVWIEFGR |
| 1320.7271 | 1320.6123 | −0.1148 | −87 | 1835 | 1846 | GDLRFVTISGQK |
| 1406.7386 | 1406.7148 | −0.0238 | −17 | 4591 | 4602 | QPVYDTTIRTGR |
| 1406.7386 | 1406.7148 | −0.0238 | −17 | 4591 | 4602 | QPVYDTTIRTGR |
| | 1406.7148 | | | | | |
| 1413.7809 | 1413.8362 | 0.0553 | 39 | 3100 | 3111 | ARQEQLELTLGR |
| 1420.7213 | 1420.736 | 0.0147 | 10 | 2884 | 2895 | TGSLEEMTQRLR |
| 1425.7156 | 1425.8483 | 0.1327 | 93 | 834 | 845 | NTISVKAVCDYR |
| 1425.7156 | 1425.8483 | 0.1327 | 93 | 834 | 845 | NTISVKAVCDYR |
| 1428.7693 | 1428.7908 | 0.0215 | 15 | 4996 | 5007 | LNDALDRLEELK |
| 1450.6996 | 1450.7076 | 0.008 | 6 | 2100 | 2110 | FEQLCLQQQEK |
| 1465.7281 | 1465.804 | 0.0759 | 52 | 4372 | 4383 | EETYNQLLDKGR |
| 1465.7316 | 1465.804 | 0.0724 | 49 | 4384 | 4397 | LMLLSRDDSGSG |
| | | | | | | SK |
| 1487.7952 | 1487.7893 | −0.0059 | −4 | 3509 | 3521 | QTTGEEVLLIQEK |
| 1502.873 | 1502.896 | 0.023 | 15 | 345 | 356 | LLEVWIEFGRIK |
| 1502.873 | 1502.896 | 0.023 | 15 | 345 | 356 | LLEVWIEFGRIK |
| 1532.6785 | 1532.8113 | 0.1328 | 87 | 3835 | 3847 | ELNPEEGEMVEEK |
| 1546.8727 | 1546.7799 | −0.0928 | −60 | 3982 | 3994 | EIKFLDVLELAEK |
| 1707.7603 | 1707.8604 | 0.1001 | 59 | 854 | 867 | NDECVLEDNSQR |
| | | | | | | TK |
| 1708.8389 | 1708.9053 | 0.0664 | 39 | 3625 | 3639 | EGLDKLVSDAN |
| | | | | | | EQYK |
| 1713.8728 | 1713.9238 | 0.051 | 30 | 3067 | 3081 | HMLEEEGTLDL |
| | | | | | | LGLK |
| 1727.9149 | 1727.9309 | 0.016 | 9 | 2116 | 2130 | KLLPQAEMFEH |
| | | | | | | LSGK |
| 1813.8942 | 1813.937 | 0.0428 | 24 | 3964 | 3977 | LDQMVFFWEDI |
| | | | | | | KAR |
| 1950.9412 | 1951.0114 | 0.0702 | 36 | 4904 | 4919 | ALIAEHQTFME |
| | | | | | | EMTRK |
| 1966.9362 | 1967.0013 | 0.0651 | 33 | 4904 | 4919 | ALIAEHQTFME |
| | | | | | | EMTRK |
| 2091.9805 | 2091.998 | 0.0175 | 8 | 461 | 476 | DENYYQLEELA |
| | | | | | | FRVMR |
| 2186.155 | 2185.9929 | −0.1621 | −74 | 1923 | 1943 | LLSDTVASDPG |
| | | | | | | VLQEQLA |
| | | | | | | TTK |
| 2186.1851 | 2186.1921 | 0.007 | 3 | 2808 | 2826 | MSELRVTLDPV |
| | | | | | | QLESSLLR |
| 2211.1301 | 2211.2874 | 0.1573 | 71 | 5151 | 5170 | STVMVRVGGG |
| | | | | | | WMALDE |
| | | | | | | FLVK |
| 2501.2268 | 2501.4001 | 0.1733 | 69 | 1304 | 1323 | FSQQYSTIVKD |
| | | | | | | YELQLMT |
| | | | | | | YK |

| | |
|---|---|
| IPI00940730 | Tax_Id = 9606 Gene_Symbol = ENOPH1 |
| | Uncharacterized protein |

Peptide Information

| | | | | Start | End | |
|---|---|---|---|---|---|---|
| Calc. Mass | Obsrv. Mass | ±da | ±ppm | Seq. | Seq. | Sequence |
| 1320.6947 | 1320.6123 | −0.0824 | −62 | 129 | 140 | AEFFADVVPAVR |
| 1479.7729 | 1479.8213 | 0.0484 | 33 | 29 | 40 | DILFPYIEENVK |
| 1579.8302 | 1579.8809 | 0.0507 | 32 | 127 | 140 | MKAEFFADVVPA |
| | | | | | | VR |
| 1745.8654 | 1745.9501 | 0.0847 | 49 | 112 | 126 | QLQGHMWRAAF |
| | | | | | | TAGR |
| | 1745.9501 | | | | | |
| 2878.4734 | 2878.4966 | 0.0232 | 8 | 162 | 187 | LLFGHSTE |
| | | | | | | GDILELVDGH |
| | | | | | | FDTKIGHK |
| 3854.9124 | 3855.2363 | 0.3239 | 84 | 149 | 183 | VYIYSSGSVEAQKLLFGH |
| | | | | | | STEGDILELVDGHFDTK |

| | |
|---|---|
| IPI00792418 | Tax_Id = 9606 Gene_Symbol = NCOR1 |
| | Nuclear receptor co-repressor isoform 1 |

Peptide Information

| | | | | Start | End | |
|---|---|---|---|---|---|---|
| Calc. Mass | Obsrv. Mass | ±da | ±ppm | Seq. | Seq. | Sequence |
| 1257.6184 | 1257.6606 | 0.0422 | 34 | 22 | 32 | SVAYMPYAEVK |
| 1257.6184 | 1257.6606 | 0.0422 | 34 | 22 | 32 | SVAYMPYAEVK |
| 1413.7195 | 1413.8362 | 0.1167 | 83 | 22 | 33 | SVAYMPYAEVKR |
| 1465.67 | 1465.804 | 0.134 | 91 | 3 | 16 | SSTSPCGTSKSP |
| | | | | | | NR |
| 1465.67 | 1465.804 | 0.134 | 91 | 3 | 16 | SSTSPCGTSKSP |
| | | | | | | NR |
| 1741.8398 | 1741.8694 | 0.0296 | 17 | 33 | 47 | RALEQEAQMHNT |
| | | | | | | AAR |
| 2199.1809 | 2199.1213 | −0.0596 | −27 | 73 | 92 | YSVPPVLQPAPH |
| | | | | | | QVITNL |
| | | | | | | PE |

| | |
|---|---|
| IPI01012199 | Tax_Id = 9606 Gene_Symbol = MACF1 |
| | Uncharacterized protein |

Protein Group

| | |
|---|---|
| IPI00256861 | Tax_Id = 9606 Gene_Symbol = MACF1 Isoform 2 of |
| | Microtubule-actin cross-linking factor 1, isoforms 1/2/3/5 |

Peptide Information

| | Obsrv. | | | Start | End | |
|---|---|---|---|---|---|---|
| Calc. Mass | Mass | ±da | ±ppm | Seq. | Seq. | Sequence |
| 842.4519 | 842.4806 | 0.0287 | 34 | 4465 | 4471 | WHVVSSK |
| 870.5229 | 870.511 | −0.0119 | −14 | 3736 | 3743 | LMALGPIR |
| 910.4265 | 910.4239 | −0.0026 | −3 | 3476 | 3482 | YSEIQDR |
| 910.4265 | 910.4239 | −0.0026 | −3 | 3476 | 3482 | YSEIQDR |
| 1021.5499 | 1021.5206 | −0.0293 | −29 | 2551 | 2558 | QQVQFMLK |
| 1021.5499 | 1021.5206 | −0.0293 | −29 | 2551 | 2558 | QQVQFMLK |
| 1170.6841 | 1170.6456 | −0.0385 | −33 | 3312 | 3321 | VVKAQIQEQK |
| 1187.6201 | 1187.673 | 0.0529 | 45 | 2757 | 2766 | NCPISAKLER |
| 1232.6117 | 1232.6179 | 0.0062 | 5 | 2659 | 2668 | QQLEETSEIR |
| 1257.6797 | 1257.6606 | −0.0191 | −15 | 1506 | 1516 | QISEQLNALNK |
| 1257.6797 | 1257.6606 | −0.0191 | −15 | 1506 | 1516 | QISEQLNALNK |
| 1261.694 | 1261.6659 | −0.0281 | −22 | 380 | 389 | LLEVWIEFGR |
| | 1261.6659 | | | | | |
| 1320.7271 | 1320.6123 | −0.1148 | −87 | 1870 | 1881 | GDLRFVTISGQK |
| 1406.7386 | 1406.7148 | −0.0238 | −17 | 4647 | 4658 | QPVYDTTIRTGR |
| 1406.7386 | 1406.7148 | −0.0238 | −17 | 4647 | 4658 | QPVYDTTIRTGR |
| 1413.7809 | 1413.8362 | 0.0553 | 39 | 3156 | 3167 | ARQEQLELTLGR |
| 1420.7213 | 1420.736 | 0.0147 | 10 | 2940 | 2951 | TGSLEEMTQRLR |
| 1425.7156 | 1425.8483 | 0.1327 | 93 | 869 | 880 | NTISVKAVCDYR |
| 1425.7156 | 1425.8483 | 0.1327 | 93 | 869 | 880 | NTISVKAVCDYR |
| 1428.7693 | 1428.7908 | 0.0215 | 15 | 5052 | 5063 | LNDALDRLEELK |
| 1450.6996 | 1450.7076 | 0.008 | 6 | 2135 | 2145 | FEQLCLQQQEK |
| 1465.7281 | 1465.804 | 0.0759 | 52 | 4428 | 4439 | EETYNQLLDKGR |
| 1465.7316 | 1465.804 | 0.0724 | 49 | 4440 | 4453 | LMLLSRDDSGSGSK |
| 1487.7952 | 1487.7893 | −0.0059 | −4 | 3565 | 3577 | QTTGEEVLLIQEK |
| 1502.873 | 1502.896 | 0.023 | 15 | 380 | 391 | LLEVWIEFGRIK |
| 1502.873 | 1502.896 | 0.023 | 15 | 380 | 391 | LLEVWIEFGRIK |
| 1532.6785 | 1532.8113 | 0.1328 | 87 | 3891 | 3903 | ELNPEEGEMVEEK |
| 1546.8727 | 1546.7799 | −0.0928 | −60 | 4038 | 4050 | EIKFLDVLELAEK |
| 1707.7603 | 1707.8604 | 0.1001 | 59 | 889 | 902 | NDECVLEDNSQRTK |
| 1708.8389 | 1708.9053 | 0.0664 | 39 | 3681 | 3695 | EGLDKLVSDANEQYK |
| 1713.8728 | 1713.9238 | 0.051 | 30 | 3123 | 3137 | HMLEEEGTLDLLGLK |
| 1727.9149 | 1727.9309 | 0.016 | 9 | 2151 | 2165 | KLLPQAEMFEHLSGK |
| 1813.8942 | 1813.937 | 0.0428 | 24 | 4020 | 4033 | LDQMVFFWEDIKAR |
| 1950.9412 | 1951.0114 | 0.0702 | 36 | 4960 | 4975 | ALIAEHQTFMEEMTRK |
| 1966.9362 | 1967.0013 | 0.0651 | 33 | 4960 | 4975 | ALIAEHQTFMEEMTRK |
| 2091.9805 | 2091.998 | 0.0175 | 8 | 496 | 511 | DENYYQLEELA |
| | | | | | | FRVMR |
| 2186.155 | 2185.9929 | −0.1621 | −74 | 1958 | 1978 | LLSDTVASDPG |
| | | | | | | VLQEQLA |
| | | | | | | TTK |
| 2186.1851 | 2186.1921 | 0.007 | 3 | 2864 | 2882 | MSELRVTLDPV |
| | | | | | | QLESSLLR |
| 2211.1301 | 2211.2874 | 0.1573 | 71 | 5207 | 5226 | STVMVRVGGG |
| | | | | | | WMALDE |
| | | | | | | FLVK |
| 2501.2268 | 2501.4001 | 0.1733 | 69 | 1339 | 1358 | FSQQYSTIVKD |
| | | | | | | YELQLMT |
| | | | | | | YK |

| | |
|---|---|
| IPI00877954 | Tax_Id = 9606 |
| | Gene_Symbol = DECR2 5 kDa protein |

Peptide Information

| | | | | Start | End | |
|---|---|---|---|---|---|---|
| Calc. Mass | Obsrv. Mass | ±da | ±ppm | Seq. | Seq. | Sequence |
| 879.4756 | 879.389 | −0.0866 | −98 | 44 | 50 | IAEIFMR |
| 1170.6089 | 1170.6456 | 0.0367 | 31 | 19 | 27 | HLFCPDLLR |
| 1413.7308 | 1413.8362 | 0.1054 | 75 | 19 | 29 | HLFCPDLLRDK |
| 2199.1743 | 2199.1213 | −0.053 | −24 | 30 | 50 | VAFITGGGSGIGF |
| | | | | | | RIAEIF MR |

| | |
|---|---|
| IPI00038378 | Tax_Id = 9606 Gene_Symbol = ENOPH1 Isoform 1 of |
| | Enolase-phosphatase E1 |

Peptide Information

| | | | | Start | End | |
|---|---|---|---|---|---|---|
| Calc. Mass | Obsrv. Mass | ±da | ±ppm | Seq. | Seq. | Sequence |
| 1320.6947 | 1320.6123 | −0.0824 | −62 | 129 | 140 | AEFFADVVPAVR |
| 1479.7729 | 1479.8213 | 0.0484 | 33 | 29 | 40 | DILFPYIEENVK |
| 1579.8302 | 1579.8809 | 0.0507 | 32 | 127 | 140 | MKAEFFADVVPA |
| | | | | | | VR |
| 1745.8654 | 1745.9501 | 0.0847 | 49 | 112 | 126 | QLQGHMWRAAF |
| | | | | | | TAGR |
| 2878.4734 | 2878.4966 | 0.0232 | 8 | 162 | 187 | LLFGHSTEGDILE |
| | | | | | | LVDGH |
| | | | | | | FDTKIGHK |
| 3854.9124 | 3855.2363 | 0.3239 | 84 | 149 | 183 | VYIYSSGSVEAQK |
| | | | | | | LLFGH |
| | | | | | | STEGDILELVDGH |
| | | | | | | FDTK |

| | |
|---|---|
| IPI00289815 | Tax_Id = 9606 Gene_Symbol = WDR7 Isoform 2 of WD |
| | repeat-containing protein 7 |

Peptide Information

| | | | | Start | End | |
|---|---|---|---|---|---|---|
| Calc. Mass | Obsrv. Mass | ±da | ±ppm | Seq. | Seq. | Sequence |
| 985.5676 | 985.5631 | −0.0045 | −5 | 868 | 877 | KLPASEGVGK |
| 985.5676 | 985.5631 | −0.0045 | −5 | 868 | 877 | KLPASEGVGK |
| 1097.5773 | 1097.5127 | −0.0646 | −59 | 1442 | 1451 | NVILMAHDGK |
| 1257.5609 | 1257.6606 | 0.0997 | 79 | 1323 | 1331 | FYMVSYYER |
| 1257.5609 | 1257.6606 | 0.0997 | 79 | 1323 | 1331 | FYMVSYYER |
| 1261.6107 | 1261.6659 | 0.0552 | 44 | 983 | 991 | WQDRCLEVR |
| 1271.6743 | 1271.6776 | 0.0033 | 3 | 1362 | 1374 | GPITAVAFAPD |
| | | | | | | GR |
| 1320.7205 | 1320.6123 | −0.1082 | −82 | 636 | 647 | SLAALKNMAH |
| | | | | | | HK |
| 1350.6294 | 1350.7144 | 0.085 | 63 | 1312 | 1322 | GLQECFPAICR |
| 1406.7097 | 1406.7148 | 0.0051 | 4 | 669 | 680 | YSHNSLMVQAIK |
| 1406.7097 | 1406.7148 | 0.0051 | 4 | 669 | 680 | YSHNSLMVQAIK |
| 1420.689 | 1420.736 | 0.047 | 33 | 285 | 297 | LPASCLPASDS |
| | | | | | | FR |
| 1713.8846 | 1713.9238 | 0.0392 | 23 | 271 | 284 | VIIWTENGQSY |
| | | | | | | IYK |
| 1951.0834 | 1951.0114 | −0.072 | −37 | 1141 | 1157 | HTCKALTFLLL |
| | | | | | | QPPSPK |
| 2185.9666 | 2185.9929 | 0.0263 | 12 | 756 | 772 | EHLLDDEEED |
| | | | | | | EEIMRQR |
| 3038.4968 | 3038.5745 | 0.0777 | 26 | 480 | 505 | YDQRYLISGGV |
| | | | | | | DFSVIIW |
| | | | | | | DIFSGEMK |
| 3854.9578 | 3855.2363 | 0.2785 | 72 | 949 | 981 | QGWSQLAAM |
| | | | | | | HCVMLPD |
| | | | | | | LLGLDKFRPPL |
| | | | | | | LEMLAR |

7 no matched protein found

| 158/G9 | Instr./Gel Origin |
|---|---|
| [1] Sample Project | |
| 20111201 | Instrument Sample |

| | | |
|---|---|---|
| Accession No. | Protein Name | Name |

| | |
|---|---|
| IPI00289815 | Tax_Id = 9606 Gene_Symbol = |
| | WDR7 Isoform 2 of WD |
| | repeat-containing protein 7 |

KH27 (SEQ ID NO. KH27) Peptide Information

| | | | | Start | End | |
|---|---|---|---|---|---|---|
| Calc. Mass | Obsrv. Mass | ±da | ±ppm | Seq. | Seq. | Sequence |
| 985.5676 | 985.5671 | −0.0005 | −1 | 868 | 877 | KLPASEGVGK |
| 1257.5609 | 1257.6626 | 0.1017 | 81 | 1323 | 1331 | FYMVSYYER |
| 1261.6107 | 1261.6564 | 0.0457 | 36 | 983 | 991 | WQDRCLEVR |
| 1261.6107 | 1261.6564 | 0.0457 | 36 | 983 | 991 | WQDRCLEVR |
| 1271.6743 | 1271.6829 | 0.0086 | 7 | 1362 | 1374 | GPITAVAFAPDGR |
| 1320.7205 | 1320.6122 | −0.1083 | −82 | 636 | 647 | SLAALKNMAHHK |
| 1320.7205 | 1320.6122 | −0.1083 | −82 | 636 | 647 | SLAALKNMAHHK |
| 1350.6294 | 1350.6978 | 0.0684 | 51 | 1312 | 1322 | GLQECFPAICR |
| 1406.7097 | 1406.7087 | −0.001 | −1 | 669 | 680 | YSHNSLMVQAIK |
| 1406.7097 | 1406.7087 | −0.001 | −1 | 669 | 680 | YSHNSLMVQAIK |
| 1420.689 | 1420.7378 | 0.0488 | 34 | 285 | 297 | LPASCLPASDSFR |
| 1713.8846 | 1713.9213 | 0.0367 | 21 | 271 | 284 | VIIWTENGQSYIYK |
| 1901.8069 | 1901.9828 | 0.1759 | 92 | 756 | 770 | EHLLDDEEEDEEI |
| | | | | | | MR |
| 1951.0834 | 1950.9768 | −0.1066 | −55 | 1141 | 1157 | HTCKALTFLLLQP |
| | | | | | | PSPK |
| 2092.2278 | 2092.0271 | −0.2007 | −96 | 1224 | 1243 | HALSLIATARPPA |
| | | | | | | FITTIAK |
| 2185.9666 | 2186.0493 | 0.0827 | 38 | 756 | 772 | EHLLDDEEEDEEI |
| | | | | | | MRQR |
| 2185.9666 | 2186.0493 | 0.0827 | 38 | 756 | 772 | EHLLDDEEEDE |
| | | | | | | EIMRQR |
| 2233.1296 | 2233.1709 | 0.0413 | 18 | 1354 | 1374 | CQTIHGHKGPI |
| | | | | | | TAVAFAP |
| | | | | | | DGR |
| 3038.4968 | 3038.5193 | 0.0225 | 7 | 480 | 505 | YDQRYLISGGV |
| | | | | | | DFSVIIW |
| | | | | | | DIFSGEMK |

| | |
|---|---|
| IPI00328832 | Tax_Id = 9606 Gene_Symbol = WDR7 Isoform 1 of WD |
| | repeat-containing protein 7 |

Peptide Information

| | | | | Start | End | |
|---|---|---|---|---|---|---|
| Calc. Mass | Obsrv. Mass | ±da | ±ppm | Seq. | Seq. | Sequence |
| 985.5676 | 985.5671 | −0.0005 | −1 | 868 | 877 | KLPASEGVGK |
| 1257.5609 | 1257.6626 | 0.1017 | 81 | 1356 | 1364 | FYMVSYYER |
| 1261.6107 | 1261.6564 | 0.0457 | 36 | 1016 | 1024 | WQDRCLEVR |
| 1261.6107 | 1261.6564 | 0.0457 | 36 | 1016 | 1024 | WQDRCLEVR |
| 1271.6743 | 1271.6829 | 0.0086 | 7 | 1395 | 1407 | GPITAVAFAPDGR |
| 1320.7205 | 1320.6122 | −0.1083 | −82 | 636 | 647 | SLAALKNMAHHK |
| 1320.7205 | 1320.6122 | −0.1083 | −82 | 636 | 647 | SLAALKNMAHHK |
| 1350.6294 | 1350.6978 | 0.0684 | 51 | 1345 | 1355 | GLQECFPAICR |
| 1406.7097 | 1406.7087 | −0.001 | −1 | 669 | 680 | YSHNSLMVQAIK |
| 1406.7097 | 1406.7087 | −0.001 | −1 | 669 | 680 | YSHNSLMVQAIK |
| 1420.689 | 1420.7378 | 0.0488 | 34 | 285 | 297 | LPASCLPASDSFR |
| 1713.8846 | 1713.9213 | 0.0367 | 21 | 271 | 284 | VIIWTENGQSYIYK |
| 1901.8069 | 1901.9828 | 0.1759 | 92 | 756 | 770 | EHLLDDEEEDEEI |
| | | | | | | MR |
| 1951.0834 | 1950.9768 | −0.1066 | −55 | 1174 | 1190 | HTCKALTFLLLQP |
| | | | | | | PSPK |
| 2092.2278 | 2092.0271 | −0.2007 | −96 | 1257 | 1276 | HALSLIATARPPA |
| | | | | | | FITTIAK |
| 2185.9666 | 2186.0493 | 0.0827 | 38 | 756 | 772 | EHLLDDEEEDEEI |
| | | | | | | MRQR |
| 2185.9666 | 2186.0493 | 0.0827 | 38 | 756 | 772 | EHLLDDEEEDEEI |
| | | | | | | MRQR |
| 2233.1296 | 2233.1709 | 0.0413 | 18 | 1387 | 1407 | CQTIHGHKGPITA |
| | | | | | | VAFAP |
| | | | | | | DGR |
| 3038.4968 | 3038.5193 | 0.0225 | 7 | 480 | 505 | YDQRYLISGGVD |
| | | | | | | FSVIIW |
| | | | | | | DIFSGEMK |

| | |
|---|---|
| IPI01008928 | Tax_Id = 9606 Gene_Symbol = - Myosin-reactive |
| | immunoglobulin heavy chain variable region (Fragment) |

Peptide Information

| | | | | Start | End | |
|---|---|---|---|---|---|---|
| Calc. Mass | Obsrv. Mass | ±da | ±ppm | Seq. | Seq. | Sequence |
| 1287.6791 | 1287.6769 | −0.0022 | −2 | 1 | 12 | EVQLVESGAEVK |
| 1320.5525 | 1320.6122 | 0.0597 | 45 | 88 | 98 | SDDTAVYYCAR |
| 1320.5525 | 1320.6122 | 0.0597 | 45 | 88 | 98 | SDDTAVYYCAR |
| 1838.8319 | 1839.0062 | 0.1743 | 95 | 24 | 38 | ASGYTFTGYYMH |
| | | | | | | WVR |
| 2092.0049 | 2092.0271 | 0.0222 | 11 | 68 | 85 | VTMTRDTTISTAY |
| | | | | | | MELSR |
| 2096.9958 | 2097.0576 | 0.0618 | 29 | 105 | 125 | IAAAGDAFDIWG |
| | | | | | | QGTMVT |

| | |
|---|---|
| IPI01012199 | Tax_Id = 9606 Gene_Symbol = MACF1 |
| | Uncharacterized protein |

Protein Group

| | |
|---|---|
| IPI00256861 | Tax_Id = 9606 Gene_Symbol = MACF1 Isoform 2 of |
| | Microtubule-actin cross-linking factor 1, isoforms 1/2/3/5 |
| | VSS |

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5264 | 0.0035 | 4 | 3736 | 3743 | LMALGPIR |
| 870.5229 | 870.5264 | 0.0035 | 4 | 3736 | 3743 | LMALGPIR |
| 910.4265 | 910.4365 | 0.01 | 11 | 3476 | 3482 | YSEIQDR |
| 1021.5499 | 1021.5289 | −0.021 | −21 | 2551 | 2558 | QQVQFMLK |
| 1021.5499 | 1021.5289 | −0.021 | −21 | 2551 | 2558 | QQVQFMLK |
| 1170.6841 | 1170.6501 | −0.034 | −29 | 3312 | 3321 | VVKAQIQEQK |
| 1187.6201 | 1187.6735 | 0.0534 | 45 | 2757 | 2766 | NCPISAKLER |
| 1187.6201 | 1187.6735 | 0.0534 | 45 | 2757 | 2766 | NCPISAKLER |
| 1225.6497 | 1225.5806 | −0.0691 | −56 | 3958 | 3968 | MPPLIPAEVDK |
| 1232.6117 | 1232.6139 | 0.0022 | 2 | 2659 | 2668 | QQLEETSEIR |
| 1257.6797 | 1257.6626 | −0.0171 | −14 | 1506 | 1516 | QISEQLNALNK |
| 1261.694 | 1261.6564 | −0.0376 | −30 | 380 | 389 | LLEVWIEFGR |
| 1261.694 | 1261.6564 | −0.0376 | −30 | 380 | 389 | LLEVWIEFGR |
| 1287.6791 | 1287.6769 | −0.0022 | −2 | 4662 | 4672 | EKTLLPEDSQK |
| 1320.7271 | 1320.6122 | −0.1149 | −87 | 1870 | 1881 | GDLRFVTISGQK |
| 1320.7271 | 1320.6122 | −0.1149 | −87 | 1870 | 1881 | GDLRFVTISGQK |
| 1406.7386 | 1406.7087 | −0.0299 | −21 | 4647 | 4658 | QPVYDTTIRTGR |
| 1406.7386 | 1406.7087 | −0.0299 | −21 | 4647 | 4658 | QPVYDTTIRTGR |
| 1413.7809 | 1413.825 | 0.0441 | 31 | 3156 | 3167 | ARQEQLELTLGR |
| 1420.7213 | 1420.7378 | 0.0165 | 12 | 2940 | 2951 | TGSLEEMTQRLR |
| 1425.7156 | 1425.8256 | 0.11 | 77 | 869 | 880 | NTISVKAVCDYR |
| 1450.6996 | 1450.6963 | −0.0033 | −2 | 2135 | 2145 | FEQLCLQQQEK |
| 1465.7281 | 1465.7937 | 0.0656 | 45 | 4428 | 4439 | EETYNQLLDKGR |
| 1465.7316 | 1465.7937 | 0.0621 | 42 | 4440 | 4453 | LMLLSRDDSGSG |
| | | | | | | SK |
| 1502.873 | 1502.8854 | 0.0124 | 8 | 380 | 391 | LLEVWIEFGRIK |
| 1532.6785 | 1532.8059 | 0.1274 | 83 | 3891 | 3903 | ELNPEEGEMVEEK |
| 1546.8727 | 1546.7936 | −0.0791 | −51 | 4038 | 4050 | EIKFLDVLELAEK |
| 1713.8728 | 1713.9213 | 0.0485 | 28 | 3123 | 3137 | HMLEEEGTLDLL |
| | | | | | | GLK |
| 1794.9636 | 1794.8539 | −0.1097 | −61 | 5106 | 5121 | QEFIDGILASKFP |
| | | | | | | TTK |
| 1838.8412 | 1839.0062 | 0.165 | 90 | 4960 | 4974 | ALIAEHQTFM |
| | | | | | | EEMTR |
| 1950.9412 | 1950.9768 | 0.0356 | 18 | 4960 | 4975 | ALIAEHQTFM |
| | | | | | | EEMTRK |
| 1966.9362 | 1966.9713 | 0.0351 | 18 | 4960 | 4975 | ALIAEHQTFME |
| | | | | | | EMTRK |
| 2092.0266 | 2092.0271 | 0.0005 | 0 | 2275 | 2293 | WLKETEGSIPP |
| | | | | | | TETSMSAK |
| 2186.155 | 2186.0493 | −0.1057 | −48 | 1958 | 1978 | LLSDTVASDPG |
| | | | | | | VLQEQLA |
| | | | | | | TTK |
| 2186.155 | 2186.0493 | −0.1057 | −48 | 1958 | 1978 | LLSDTVASDPG |
| | | | | | | VLQEQLA |
| | | | | | | TTK |
| 2200.0632 | 2200.0908 | 0.0276 | 13 | 4015 | 4031 | EIQDKLDQMVF |
| | | | | | | FWEDIK |
| 2233.1135 | 2233.1709 | 0.0574 | 26 | 2462 | 2481 | EALAGLLVTYP |
| | | | | | | NSQEAEN |
| | | | | | | WK |
| 2299.0217 | 2299.2339 | 0.2122 | 92 | 3068 | 3088 | EMFSQLADLDD |
| | | | | | | ELDGMG |
| | | | | | | AIGR |
| 2501.2268 | 2501.3357 | 0.1089 | 44 | 1339 | 1358 | FSQQYSTIVKD |
| | | | | | | YELQLMT |
| | | | | | | YK |

| | |
|---|---|
| IPI00977191 | Tax_Id = 9606 Gene_Symbol = NASP Uncharacterized |
| | protein |

Peptide Information

| Calc. Mass | Obsrv. Mass | ±da | ±ppm | Start Seq. | End Seq. | |
|---|---|---|---|---|---|---|
| 912.4322 | 912.4548 | 0.0226 | 25 | 40 | 46 | |
| 1851.9004 | 1852.0027 | 0.1023 | 55 | 2 | 20 | AMESTATAAVAA |
| | | | | | | ELVSADK |
| 1966.946 | 1966.9713 | 0.0253 | 13 | 1 | 20 | MAMESTATAAVA |
| | | | | | | AELVS |
| | | | | | | ADK |
| 2299.0906 | 2299.2339 | 0.1433 | 62 | 2 | 24 | AMESTATAAVAA |
| | | | | | | ELVSAD |
| | | | | | | KMSGR |

| | |
|---|---|
| IPI01018940 | Tax_Id = 9606 Gene _Symbol = MACF1 Isoform 3 of |
| | Microtubule-actin cross-linking factor 1, |
| | isoforms 1/2/3/5 |

Peptide Information

| | | | | | End | |
|---|---|---|---|---|---|---|
| Calc. Mass | Obsrv. Mass | ±da | ±ppm | Start Seq. | Seq. | Sequence |
| 870.5229 | 870.5264 | 0.0035 | 4 | 3680 | 3687 | LMALGPIR |
| 870.5229 | 870.5264 | 0.0035 | 4 | 3680 | 3687 | LMALGPIR |
| 910.4265 | 910.4365 | 0.01 | 11 | 3420 | 3426 | YSEIQDR |
| 1021.5499 | 1021.5289 | −0.021 | −21 | 2495 | 2502 | QQVQFMLK |
| 1021.5499 | 1021.5289 | −0.021 | −21 | 2495 | 2502 | QQVQFMLK |
| 1170.6841 | 1170.6501 | −0.034 | −29 | 3256 | 3265 | VVKAQIQEQK |
| 1187.6201 | 1187.6735 | 0.0534 | 45 | 2701 | 2710 | NCPISAKLER |
| 1187.6201 | 1187.6735 | 0.0534 | 45 | 2701 | 2710 | NCPISAKLER |
| 1225.6497 | 1225.5806 | −0.0691 | −56 | 3902 | 3912 | MPPLIPAEVDK |
| | 1225.5806 | | | | | |
| 1232.6117 | 1232.6139 | 0.0022 | 2 | 2603 | 2612 | QQLEETSEIR |
| 1257.6797 | 1257.6626 | −0.0171 | −14 | 1471 | 1481 | QISEQLNALNK |
| 1261.694 | 1261.6564 | −0.0376 | −30 | 345 | 354 | LLEVWIEFGR |
| 1261.694 | 1261.6564 | −0.0376 | −30 | 345 | 354 | LLEVWIEFGR |
| 1287.6791 | 1287.6769 | −0.0022 | −2 | 4606 | 4616 | EKTLLPEDSQK |
| 1320.7271 | 1320.6122 | −0.1149 | −87 | 1835 | 1846 | GDLRFVTISGQK |
| 1320.7271 | 1320.6122 | −0.1149 | −87 | 1835 | 1846 | GDLRFVTISGQK |
| 1406.7386 | 1406.7087 | −0.0299 | −21 | 4591 | 4602 | QPVYDTTIRTGR |
| 1406.7386 | 1406.7087 | −0.0299 | −21 | 4591 | 4602 | QPVYDTTIRTGR |
| 1413.7809 | 1413.825 | 0.0441 | 31 | 3100 | 3111 | ARQEQLELTLGR |
| 1420.7213 | 1420.7378 | 0.0165 | 12 | 2884 | 2895 | TGSLEEMTQRLR |
| 1425.7156 | 1425.8256 | 0.11 | 77 | 834 | 845 | NTISVKAVCDYR |
| 1450.6996 | 1450.6963 | −0.0033 | −2 | 2100 | 2110 | FEQLCLQQQEK |
| 1465.7281 | 1465.7937 | 0.0656 | 45 | 4372 | 4383 | EETYNQLLDKGR |
| 1465.7316 | 1465.7937 | 0.0621 | 42 | 4384 | 4397 | LMLLSRDDSGS |
| | | | | | | GSK |
| 1502.873 | 1502.8854 | 0.0124 | 8 | 345 | 356 | LLEVWIEFGRIK |
| 1532.6785 | 1532.8059 | 0.1274 | 83 | 3835 | 3847 | ELNPEEGEMVE |
| | | | | | | EK |
| 1546.8727 | 1546.7936 | −0.0791 | −51 | 3982 | 3994 | EIKFLDVLELAEK |
| 1713.8728 | 1713.9213 | 0.0485 | 28 | 3067 | 3081 | HMLEEEGTLDL |
| | | | | | | LGLK |
| 1794.9636 | 1794.8539 | −0.1097 | −61 | 5050 | 5065 | QEFIDGILASKF |
| | | | | | | PTTK |
| 1838.8412 | 1839.0062 | 0.165 | 90 | 4904 | 4918 | ALIAEHQTFME |
| | | | | | | EMTR |
| 1950.9412 | 1950.9768 | 0.0356 | 18 | 4904 | 4919 | ALIAEHQTFME |
| | | | | | | EMTRK |
| 1966.9362 | 1966.9713 | 0.0351 | 18 | 4904 | 4919 | ALIAEHQTFME |
| | | | | | | EMTRK |
| 2092.0266 | 2092.0271 | 0.0005 | 0 | 2240 | 2258 | WLKETEGSIPP |
| | | | | | | TETSMSAK |
| 2186.155 | 2186.0493 | −0.1057 | −48 | 1923 | 1943 | LLSDTVASDPG |
| | | | | | | VLQEQLA |
| | | | | | | TTK |
| 2186.155 | 2186.0493 | −0.1057 | −48 | 1923 | 1943 | LLSDTVASDPG |
| | | | | | | VLQEQLA |
| | | | | | | TTK |
| 2200.0632 | 2200.0908 | 0.0276 | 13 | 3959 | 3975 | EIQDKLDQMVF |
| | | | | | | FWEDIK |
| 2233.1135 | 2233.1709 | 0.0574 | 26 | 2406 | 2425 | EALAGLLVTYP |
| | | | | | | NSQEAEN |
| | | | | | | WK |
| 2299.0217 | 2299.2339 | 0.2122 | 92 | 3012 | 3032 | EMFSQLADLDD |
| | | | | | | ELDGMG |
| | | | | | | AIGR |
| 2501.2268 | 2501.3357 | 0.1089 | 44 | 1304 | 1323 | FSQQYSTIVKD |
| | | | | | | YELQLMT |
| | | | | | | YK |

| | |
|---|---|
| IPI01018834 | Tax_Id = 9606 Gene_Symbol = MACF1 Uncharacterized |
| | protein |

Protein Group

| | |
|---|---|
| IPI00478226 | Tax_Id = 9606 Gene_Symbol = MACF1 Isoform 5 of |
| | Microtubule-actin cross-linking factor 1, isoforms 1/2/3/5 |

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5264 | 0.0035 | 4 | 3606 | 3613 | LMALGPIR |
| 870.5229 | 870.5264 | 0.0035 | 4 | 3606 | 3613 | LMALGPIR |
| 1021.5499 | 1021.5289 | −0.021 | −21 | 2530 | 2537 | QQVQFMLK |
| 1021.5499 | 1021.5289 | −0.021 | −21 | 2530 | 2537 | QQVQFMLK |
| 1170.6841 | 1170.6501 | −0.034 | −29 | 3291 | 3300 | VVKAQIQEQK |
| 1187.6201 | 1187.6735 | 0.0534 | 45 | 2736 | 2745 | NCPISAKLER |
| 1187.6201 | 1187.6735 | 0.0534 | 45 | 2736 | 2745 | NCPISAKLER |
| 1225.6497 | 1225.5806 | −0.0691 | −56 | 3828 | 3838 | MPPLIPAEVDK |
| 1232.6117 | 1232.6139 | 0.0022 | 2 | 2638 | 2647 | QQLEETSEIR |
| 1257.6797 | 1257.6626 | −0.0171 | −14 | 1506 | 1516 | QISEQLNALNK |
| 1261.694 | 1261.6564 | −0.0376 | −30 | 380 | 389 | LLEVWIEFGR |
| 1261.694 | 1261.6564 | −0.0376 | −30 | 380 | 389 | LLEVWIEFGR |
| 1287.6791 | 1287.6769 | −0.0022 | −2 | 4532 | 4542 | EKTLLPEDSQK |
| 1320.7271 | 1320.6122 | −0.1149 | −87 | 1870 | 1881 | GDLRFVTISGQK |
| 1320.7271 | 1320.6122 | −0.1149 | −87 | 1870 | 1881 | GDLRFVTISGQK |
| 1406.7386 | 1406.7087 | −0.0299 | −21 | 4517 | 4528 | QPVYDTTIRTGR |
| 1406.7386 | 1406.7087 | −0.0299 | −21 | 4517 | 4528 | QPVYDTTIRTGR |
| 1413.7809 | 1413.825 | 0.0441 | 31 | 3135 | 3146 | ARQEQLELTLGR |
| 1420.7213 | 1420.7378 | 0.0165 | 12 | 2919 | 2930 | TGSLEEMTQRLR |
| 1425.7156 | 1425.8256 | 0.11 | 77 | 869 | 880 | NTISVKAVCDYR |
| 1450.6996 | 1450.6963 | −0.0033 | −2 | 2135 | 2145 | FEQLCLQQQEK |
| 1465.7281 | 1465.7937 | 0.0656 | 45 | 4298 | 4309 | EETYNQLLDKGR |
| 1465.7316 | 1465.7937 | 0.0621 | 42 | 4310 | 4323 | LMLLSRDDSGSG |
| | | | | | | SK |
| 1502.873 | 1502.8854 | 0.0124 | 8 | 380 | 391 | LLEVWIEFGRIK |
| 1532.6785 | 1532.8059 | 0.1274 | 83 | 3761 | 3773 | ELNPEEGEMVEEK |
| 1546.8727 | 1546.7936 | −0.0791 | −51 | 3908 | 3920 | EIKFLDVLELAEK |
| 1713.8728 | 1713.9213 | 0.0485 | 28 | 3102 | 3116 | HMLEEEGTLDLL |
| | | | | | | GLK |
| 1794.9636 | 1794.8539 | −0.1097 | −61 | 4976 | 4991 | QEFIDGILASKFP |
| | | | | | | TTK |
| 1838.8412 | 1839.0062 | 0.165 | 90 | 4830 | 4844 | ALIAEHQTFMEE |
| | | | | | | MTR |
| 1950.9412 | 1950.9768 | 0.0356 | 18 | 4830 | 4845 | ALIAEHQTFMEE |
| | | | | | | MTRK |
| 1966.9362 | 1966.9713 | 0.0351 | 18 | 4830 | 4845 | ALIAEHQTFMEE |
| | | | | | | MTRK |
| 2092.0266 | 2092.0271 | 0.0005 | 0 | 2275 | 2293 | WLKETEGSIPP |
| | | | | | | TETSMSA K |
| 2186.155 | 2186.0493 | −0.1057 | −48 | 1958 | 1978 | LLSDTVASDPG |
| | | | | | | VLQEQLA |
| | | | | | | TTK |
| 2186.155 | 2186.0493 | −0.1057 | −48 | 1958 | 1978 | LLSDTVASDPG |
| | | | | | | VLQEQLA |
| | | | | | | TTK |
| 2200.0632 | 2200.0908 | 0.0276 | 13 | 3885 | 3901 | EIQDKLDQMVF |
| | | | | | | FWEDIK |
| 2233.1135 | 2233.1709 | 0.0574 | 26 | 2441 | 2460 | EALAGLLVTYP |
| | | | | | | NSQEAEN |
| | | | | | | WK |
| 2299.0217 | 2299.2339 | 0.2122 | 92 | 3047 | 3067 | EMFSQLADLDD |
| | | | | | | ELDGMG |
| | | | | | | AIGR |
| | | | | | | FSQQYSTIVKD |
| 2501.2268 | 2501.3357 | 0.1089 | 44 | 1339 | 1358 | YELQLMT |
| | | | | | | YK |

| | |
|---|---|
| IPI00007676 | Tax_Id = 9606 Gene_Symbol = HSD17B12 Estradiol |
| | 17-beta-dehydrogenase 12 |

Peptide Information

| | | | | Start | End | |
|---|---|---|---|---|---|---|
| Calc. Mass | Obsrv. Mass | ±da | ±ppm | Seq. | Seq. | Sequence |
| 910.4516 | 910.4365 | −0.0151 | −17 | 65 | 72 | SYAEELAK |
| 1170.6517 | 1170.6501 | −0.0016 | −1 | 26 | 35 | ISYSLFTALR |
| 1225.6028 | 1225.5806 | −0.0222 | −18 | 293 | 302 | IVMNMNKSTR |
| 1261.6635 | 1261.6564 | −0.0071 | −6 | 85 | 95 | DKLDQVSSEIK |
| 1261.6635 | 1261.6564 | −0.0071 | −6 | 85 | 95 | DKLDQVSSEIK |
| 1320.7014 | 1320.6122 | −0.0892 | −68 | 157 | 167 | MININILSVCK |
| 1320.7014 | 1320.6122 | −0.0892 | −68 | 157 | 167 | MININILSVCK |
| 1967.1365 | 1966.9713 | −0.1652 | −84 | 224 | 241 | GVFVQSVLPYFV |
| | | | | | | ATKLAK |
| 2691.4065 | 2691.3652 | −0.0413 | −15 | 157 | 179 | MININILSVCKMT |
| | | | | | | QLVLPG |
| | | | | | | MVER |
| 2707.4014 | 2707.4404 | 0.039 | 14 | 157 | 179 | MININILSVCKMT |
| | | | | | | QLVLPG |
| | | | | | | MVER |

| | |
|---|---|
| IPI00021304 | Tax_id = 9606 Gene_Symbol = KRT2 Keratin, type II |
| | cytoskeletal 2 epidermal |

Peptide Information

| | | | | Start | | |
|---|---|---|---|---|---|---|
| Calc. Mass | Obsrv. Mass | ±da | ±ppm | Seq. | End Seq. | Sequence |
| 910.4152 | 910.4365 | 0.0213 | 23 | 274 | 280 | YEDEINK |
| 985.5789 | 985.5671 | −0.0118 | −12 | 460 | 467 | EDLARLLR |
| 1254.6074 | 1254.6842 | 0.0768 | 61 | 21 | 34 | GFSSGSAVVSGG |
| | | | | | | SR |
| 1287.6111 | 1287.6769 | 0.0658 | 51 | 35 | 45 | RSTSSFSCLSR |
| 1320.5829 | 1320.6122 | 0.0293 | 22 | 46 | 61 | HGGGGGGFGGG |
| | | | | | | GFGSR |
| 1320.5829 | 1320.6122 | 0.0293 | 22 | 46 | 61 | HGGGGGGFGGG |
| | | | | | | GFGSR |
| 1740.7057 | 1740.7649 | 0.0592 | 34 | 531 | 550 | GSSSGGGYSS |
| | | | | | | GSSSYGS |
| | | | | | | GGR |
| 1745.8235 | 1745.9114 | 0.0879 | 50 | 422 | 436 | QCKNVQDAIAD |
| | | | | | | AEQR |
| 1838.9144 | 1839.0062 | 0.0918 | 50 | 71 | 92 | SISISVAGGGG |
| | | | | | | GFGAAG |
| | | | | | | GFGGR |
| 2384.2166 | 2384.1665 | −0.0501 | −21 | 468 | 487 | DYQELMNVKLA |
| | | | | | | LDVEIAT |
| | | | | | | YR |

| | |
|---|---|
| IPI00976163 | Tax_id = 9606 Gene_Symbol = LOC731282 hypothetical |
| | protein LOC731282 |

Peptide Information

| | Obsrv. | | | Start | | |
|---|---|---|---|---|---|---|
| Calc. Mass | Mass | ±da | ±ppm | Seq. | End Seq. | Sequence |
| 856.4747 | 856.5074 | 0.0327 | 38 | 297 | 304 | NPGSLRGR |
| 912.4356 | 912.4548 | 0.0192 | 21 | 170 | 176 | LETHPCR |
| 985.5425 | 985.5671 | 0.0246 | 25 | 9 | 18 | GSIGQSAIPR |
| 1350.7311 | 1350.6978 | −0.0333 | −25 | 119 | 130 | SPCPIRSPLPAR |
| 1745.8929 | 1745.9114 | 0.0185 | 11 | 82 | 98 | ASAPWASLSTR |
| | | | | | | ADSGLR |
| 1901.975 | 1901.9828 | 0.0078 | 4 | 1 | 18 | MSPLETNKGSI |
| | | | | | | GQSAIPR |
| 2384.2722 | 2384.1665 | −0.1057 | −44 | 238 | 259 | ATSASLPQETP |
| | | | | | | FALSVVW |
| | | | | | | APRR |

| | Number of | Name of |
|---|---|---|
| Product | result | protein |
| HemoRAAS | 1 | Tax_Id = 9606 Gene_Symbol = ALB Uncharacterized protein |
| HemoRAAS | 2 | Tax_Id = 9606 Gene_Symbol = ALB Uncharacterized protein |
| FNG | 3 | Tax_Id = 9606 Gene_Symbol = FGG Isoform Gamma-A of |
| | | Fibrinogen gamma chain |
| FNG | 4 | Tax_Id = 9606 Gene_Symbol = FGG Isoform Gamma-A of |
| | | Fibrinogen gamma chain |
| FNG | 5 | Tax_Id = 9606 Gene_Symbol = KRT1 Keratin, type II cytoskeletal 1 |
| IVIG | 6 | Tax_Id = 9606 Gene_Symbol = CFH Isoform 1 of Complement |
| | | factor H |
| IVIG | 7 | NA |
| IVIG | 8 | Tax_Id = 9606 Gene_Symbol = IGHM Full-length cDNA clone |
| | | CS0DD006YL02 of Neuroblastoma of |
| | | Homo |
| IVIG | 9 | Tax_Id = 9606 Gene_Symbol = FGB Fibrinogen beta chain |
| IVIG | 10 | Tax_Id = 9606 Gene_Symbol = APOA1 Apolipoprotein A-I |
| AFCC | 11 | Tax_Id = 9606 Gene_Symbol = C3 Complement C3 (Fragment) |
| Fr 3-2 | 12 | Tax_Id = 9606 Gene_Symbol = ALB Uncharacterized protein |
| Fr3 | 13 | Tax_Id = 9606 Gene_Symbol = FGB Fibrinogen beta chain |
| AFCC | 14 | Tax_Id = 9606 Gene_Symbol = FGB Fibrinogen beta chain |
| FS-TB | 15 | Tax_Id = 9606 Gene_Symbol = IGHV4-31; IGHG1 Putative |
| | | uncharacterized protein DKFZp686P15220 |
| Fr3 | 16 | Tax_Id = 9606 Gene_Symbol = FGB fibrinogen beta chain isoform |
| | | 2 preproprotein |
| FS-TB | 17 | Tax_Id = 9606 Gene_Symbol = FGG Isoform Gamma-A of |
| AFOD | 18 | Tax_Id = 9606 Gene_Symbol = TF Serotransferrin |
| AFOD | 19 | Tax_Id = 9606 Gene_Symbol = ACTC1 Actin, alpha cardiac |
| | | muscle 1 |
| AFOD | 20 | Tax_Id = 9606 Gene_Symbol = APOA1 Apolipoprotein A-I |
| HA | 21 | Tax_Id = 9606 Gene_Symbol = ACTC1 Actin, alpha cardiac |
| | | muscle 1 |
| IVIG | 22 | Tax_Id = 9606 Gene_Symbol = IGHV4-31; IGHG1 Putative |
| | | uncharacterized protein DKFZp686G11190 |
| FS-FNG | 23 | Tax_Id = 9606 Gene_Symbol = ACTG1 cDNA FLJ57283, |
| | | highly similar to Actin, |
| | | cytoplasmic 2 |
| FS-TB | 24 | Tax_Id = 9606 Gene_Symbol = HRG Histidine-rich glycoprotein |
| FS-TB | 25 | Tax_Id = 9606 Gene_Symbol = HRG Histidine-rich glycoprotein |
| AFCC | 26 | Tax_Id = 9606 Gene_Symbol = ALB 23 kDa protein |

P12008

| No. | sample | Target# | Success |
|---|---|---|---|
| 1 | 1-76 | L1 | Y |
| 2 | 1-79 | L2 | Y |
| 3 | 2-66 | L3 | Y |
| 4 | 2-68 | L4 | Y |
| 5 | 2-313 | L5 | Y |
| 6 | 3-281 | L6 | Y |
| 7 | 3-285 | L7 | N |
| 8 | 3-303 | L8 | Y |
| 9 | 3-356 | L9 | Y |
| 10 | 3-419 | L10 | Y |
| 11 | 4-97 | L11 | Y |
| 12 | 4-108 | L12 | Y |
| 13 | 4-114 | L13 | Y |
| 14 | 4-145 | L14 | Y |
| 15 | 4-168 | L15 | Y |
| 16 | 4-136 | L16 | Y |
| 17 | 4-182 | L17 | Y |
| 18 | 6-187 | L19 | Y |
| 19 | 6-213 | L19 | Y |
| 20 | 6-284 | L20 | Y |
| 21 | 10-120 | L21 | Y |
| 22 | 11-234 | L22 | Y |
| 23 | 14-206 | L23 | Y |
| 24 | 17-38 | L24 | Y |
| 25 | 17-42 | M1 | Y |
| 26 | 12-116 | M2 | Y |

| | | Date of | |
|---|---|---|---|
| Database | Number of sequences | fasta file | Dye method |
| IPI_human_v3.87 | 91464 | 2011-10-9 | blue |

The following are the sequence of these 26 newfound proteins in 2 in HemoRAAS®, 3 in FibroRAAS®, 5 in GammaRAAS®, 3 in AFCC®, 1 in Fraction 3-2, 2 in Fraction 3, 4 in FibingluRAAS® (Thrombin), 1 in FibingluRAAS® (High concentrate Fibrinogen), 3 in AFOD® and 1 in AlbuRAAS® from Human Plasma and their name KH28 through KH52.

1—KH28 (SEQ ID NO. KH28)

| 273/L1 | Instr./Gel Origin |
|---|---|
| [1] Sample Project | |
| 20120229 | Instrument Sample |

| | | |
|---|---|---|
| Accession No. | Protein Name | Name |

| | |
|---|---|
| IPI00022434 | Tax_Id = 9606 Gene_Symbol = |
| | ALB Uncharacterized protein |

Peptide Information

| | Obsrv. | | | Start | | |
|---|---|---|---|---|---|---|
| Calc. Mass | Mass | ±da | ±ppm | Seq. | End Seq. | Sequence |
| 875.5098 | 875.5438 | 0.034 | 39 | 243 | 249 | LSQRFPK |
| 927.4934 | 927.5287 | 0.0353 | 38 | 162 | 168 | YLYEIAR |
| 927.4934 | 927.5287 | 0.0353 | 38 | 162 | 168 | YLYEIAR |
| 960.5625 | 960.5985 | 0.036 | 37 | 427 | 434 | FQNALLVR |
| 1055.5884 | 1055.629 | 0.0406 | 38 | 161 | 168 | KYLYEIAR |
| 1074.5426 | 1074.5784 | 0.0358 | 33 | 206 | 214 | LDELRDE |
| | | | | | | GK |
| 1138.498 | 1138.537 | 0.039 | 34 | 500 | 508 | CCTESLV |
| | | | | | | NR |
| 1149.615 | 1149.6412 | 0.0262 | 23 | 66 | 75 | LVNEVTE |
| | | | | | | FAK |
| 1149.615 | 1149.6412 | 0.0262 | 23 | 66 | 75 | LVNEVTE |
| | | | | | | FAK |
| 1226.6052 | 1226.6315 | 0.0263 | 21 | 35 | 44 | FKDLGEE |
| | | | | | | NFK |
| 1342.6348 | 1342.6599 | 0.0251 | 19 | 570 | 581 | AVMDDFA |
| | | | | | | AFVEK |
| 1342.6348 | 1342.6599 | 0.0251 | 19 | 570 | 581 | AVMDDFA |
| | | | | | | AFVEK |
| 1358.6298 | 1358.6572 | 0.0274 | 20 | 570 | 581 | AVMDDFA |
| | | | | | | AFVEK |
| 1371.5668 | 1371.5984 | 0.0316 | 23 | 187 | 198 | AAFTECC |
| | | | | | | QAADK |
| 1434.5334 | 1434.5642 | 0.0308 | 21 | 106 | 117 | ETYGEMADCC |
| | | | | | | AK |
| 1443.6421 | 1443.6724 | 0.0303 | 21 | 287 | 298 | YICENQDSISSK |
| 1498.5785 | 1498.6459 | 0.0674 | 45 | 76 | 88 | TCVADESAEN |
| | | | | | | CDK |
| 1511.8429 | 1511.8618 | 0.0189 | 13 | 439 | 452 | VPQVSTPTLV |
| | | | | | | EVSR |
| 1546.7968 | 1546.816 | 0.0192 | 12 | 299 | 310 | LKECCEKPLLEK |
| 1552.5978 | 1552.6199 | 0.0221 | 14 | 384 | 396 | CCAAADPHEC |
| | | | | | | YAK |
| 1623.7876 | 1623.8287 | 0.0411 | 25 | 348 | 360 | DVFLGMFLYE |
| | | | | | | YAR |
| 1627.6904 | 1627.7432 | 0.0528 | 32 | 585 | 598 | ADDKETCFAE |
| | | | | | | EGQK |
| 1639.9379 | 1639.9312 | −0.0067 | −4 | 438 | 452 | KVPQVSTPTL |
| | | | | | | VEVSR |
| 1639.9379 | 1639.9312 | −0.0067 | −4 | 438 | 452 | KVPQVSTPTL |
| | | | | | | VEVSR |
| 1657.7527 | 1657.7694 | 0.0167 | 10 | 414 | 426 | QNCELFEQLG |
| | | | | | | EYK |
| 1714.7966 | 1714.822 | 0.0254 | 15 | 118 | 130 | QEPERNECFL |
| | | | | | | QHK |
| 1910.9318 | 1910.9443 | 0.0125 | 7 | 509 | 524 | RPCFSALEVD |
| | | | | | | ETYVPK |
| 1910.9318 | 1910.9443 | 0.0125 | 7 | 509 | 524 | RPCFSALEVD |
| | | | | | | ETYVPK |
| 1932.0372 | 1932.0525 | 0.0153 | 8 | 89 | 105 | SLHTLFGDKL |
| | | | | | | CTVATLR |
| 1996.9294 | 1996.9454 | 0.016 | 8 | 123 | 138 | NECFLQHKDD |
| | | | | | | NPNLPR |
| 2045.0955 | 2045.1028 | 0.0073 | 4 | 397 | 413 | VFDEFKPLVE |
| | | | | | | EPQNLIK |
| 2045.0955 | 2045.1028 | 0.0073 | 4 | 397 | 413 | VFDEFKPLVE |
| | | | | | | EPQNLIK |
| 2086.8376 | 2086.8652 | 0.0276 | 13 | 265 | 281 | VHTECCHGDL |
| | | | | | | LECADDR |
| 2124.9875 | 2124.9919 | 0.0044 | 2 | 187 | 205 | AAFTECCQAA |
| | | | | | | DKAACLLPK |
| 2260.0227 | 2260.0281 | 0.0054 | 2 | 525 | 543 | EFNAETFTFH |
| | | | | | | ADICTLSEK |
| 2545.1665 | 2545.1902 | 0.0237 | 9 | 525 | 545 | EFNAETFTFH |
| | | | | | | ADICTLSEK |
| | | | | | | ER |
| 2545.1665 | 2545.1902 | 0.0237 | 9 | 525 | 545 | EFNAETFTFH |
| | | | | | | ADICTLSEK |
| | | | | | | ER |
| 2585.1177 | 2585.145 | 0.0273 | 11 | 265 | 286 | VHTECCHGDL |
| | | | | | | LECADDR |
| | | | | | | ADLAK |
| 2585.1177 | 2585.145 | 0.0273 | 11 | 265 | 286 | VHTECCHGDL |
| | | | | | | LECADDR |
| | | | | | | ADLAK |
| 2599.2974 | 2599.2307 | −0.0667 | −26 | 414 | 434 | QNCELFEQLG |
| | | | | | | EYKFQNA |
| | | | | | | LLVR |
| 2650.2642 | 2650.2622 | −0.002 | −1 | 139 | 160 | LVRPEVDVMC |
| | | | | | | TAFHDNE |
| | | | | | | ETFLK |
| 2778.3589 | 2778.3748 | 0.0159 | 6 | 139 | 161 | LVRPEVDVMC |
| | | | | | | TAFHDNE |
| | | | | | | ETFLKK |
| 2778.3589 | 2778.3748 | 0.0159 | 6 | 139 | 161 | LVRPEVDVMC |
| | | | | | | TAFHDNE |
| | | | | | | ETFLKK |
| 2794.354 | 2794.363 | 0.009 | 3 | 139 | 161 | LVRPEVDVMC |
| | | | | | | TAFHDNE |
| | | | | | | ETFLKK |
| 2974.3445 | 2974.3828 | 0.0383 | 13 | 311 | 337 | SHCIAEVEND |
| | | | | | | EMPADLPS |
| | | | | | | LAADFVESK |

2—KH29 (SEQ ID NO. KH29)

| 274/L2 | Instr./Gel Origin |
|---|---|
| [1] Sample Project | |
| 20120229 | Instrument Sample |

| | | |
|---|---|---|
| Accession No. | Protein Name | Name |

| | |
|---|---|
| IPI00022434 | Tax_Id = 9606 Gene_Symbol = |
| | ALB Uncharacterized protein |

Peptide Information

| | | | | Start | End | |
|---|---|---|---|---|---|---|
| Calc. Mass | Obsrv. Mass | ±da | ±ppm | Seq. | Seq. | Sequence |
| 875.5098 | 875.5159 | 0.0061 | 7 | 243 | 249 | LSQRFPK |
| 927.4934 | 927.503 | 0.0096 | 10 | 162 | 168 | YLYEIAR |
| 927.4934 | 927.503 | 0.0096 | 10 | 162 | 168 | YLYEIAR |
| 960.5625 | 960.5728 | 0.0103 | 11 | 427 | 434 | FQNALLVR |
| 1019.5785 | 1019.5828 | 0.0043 | 4 | 234 | 242 | AFKAWAVAR |
| 1055.5884 | 1055.6036 | 0.0152 | 14 | 161 | 168 | KYLYEIAR |
| 1074.5426 | 1074.5536 | 0.011 | 10 | 206 | 214 | LDELRDEGK |
| 1083.5946 | 1083.6024 | 0.0078 | 7 | 162 | 169 | YLYEIARR |
| 1138.498 | 1138.4996 | 0.0016 | 1 | 500 | 508 | CCTESLVNR |
| 1149.615 | 1149.6121 | −0.0029 | −3 | 66 | 75 | LVNEVTEFAK |
| 1149.615 | 1149.6121 | −0.0029 | −3 | 66 | 75 | LVNEVTEFAK |
| 1226.6052 | 1226.6 | −0.0052 | −4 | 35 | 44 | FKDLGEENFK |
| 1311.7419 | 1311.7374 | −0.0045 | −3 | 362 | 372 | HPDYSVVLLLR |
| 1342.6348 | 1342.6281 | −0.0067 | −5 | 570 | 581 | AVMDDFAAFVEK |
| 1342.6348 | 1342.6281 | −0.0067 | −5 | 570 | 581 | AVMDDFAAFVEK |
| 1358.6298 | 1358.6241 | −0.0057 | −4 | 570 | 581 | AVMDDFAAFVEK |
| 1371.5668 | 1371.5563 | −0.0105 | −8 | 187 | 198 | AAFTECC |
| | | | | | | QAADK |
| 1434.5334 | 1434.5201 | −0.0133 | −9 | 106 | 117 | ETYGEMA |
| | | | | | | DCCAK |
| 1443.6421 | 1443.639 | −0.0031 | −2 | 287 | 298 | YICENQD |
| | | | | | | SISSK |
| 1450.5283 | 1450.5404 | 0.0121 | 8 | 106 | 117 | ETYGEMA |
| | | | | | | DCCAK |
| 1467.8431 | 1467.8247 | −0.0184 | −13 | 361 | 372 | RHPDYSV |
| | | | | | | VLLLR |
| 1467.8431 | 1467.8247 | −0.0184 | −13 | 361 | 372 | RHPDYSVVLL |
| | | | | | | LR |
| 1511.8429 | 1511.8169 | −0.026 | −17 | 439 | 452 | VPQVSTPTLV |
| | | | | | | EVSR |
| 1546.7968 | 1546.7704 | −0.0264 | −17 | 299 | 310 | LKECCEKPLLEK |
| 1552.5978 | 1552.5833 | −0.0145 | −9 | 384 | 396 | CCAAADPHEC |
| | | | | | | YAK |
| 1552.5978 | 1552.5833 | −0.0145 | −9 | 384 | 396 | CCAAADPHEC |
| | | | | | | YAK |
| 1623.7876 | 1623.7695 | −0.0181 | −11 | 348 | 360 | DVFLGMFLYE |
| | | | | | | YAR |
| 1627.6904 | 1627.7004 | 0.01 | 6 | 585 | 598 | ADDKETCFAE |
| | | | | | | EGQK |
| 1639.9379 | 1639.8976 | −0.0403 | −25 | 438 | 452 | KVPQVSTPTL |
| | | | | | | VEVSR |
| 1639.9379 | 1639.8976 | −0.0403 | −25 | 438 | 452 | KVPQVSTPTL |
| | | | | | | VEVSR |
| 1657.7527 | 1657.7295 | −0.0232 | −14 | 414 | 426 | QNCELFEQLG |
| | | | | | | EYK |
| 1662.7251 | 1662.7451 | 0.02 | 12 | 185 | 198 | YKAAFTECCQ |
| | | | | | | AADK |
| 1714.7966 | 1714.7606 | −0.036 | −21 | 118 | 130 | QEPERNECFL |
| | | | | | | QHK |
| 1910.9318 | 1910.8839 | −0.0479 | −25 | 509 | 524 | RPCFSALEVD |
| | | | | | | ETYVPK |
| 1910.9318 | 1910.8839 | −0.0479 | −25 | 509 | 524 | RPCFSALEVD |
| | | | | | | ETYVPK |
| 1932.0372 | 1931.9843 | −0.0529 | −27 | 89 | 105 | SLHTLFGDKL |
| | | | | | | CTVATLR |
| 1996.9294 | 1996.8815 | −0.0479 | −24 | 123 | 138 | NECFLQHKDD |
| | | | | | | NPNLPR |
| 2045.0955 | 2045.0405 | −0.055 | −27 | 397 | 413 | VFDEFKPLVE |
| | | | | | | EPQNLIK |
| 2045.0955 | 2045.0405 | −0.055 | −27 | 397 | 413 | VFDEFKPLVE |
| | | | | | | EPQNLIK |
| 2086.8376 | 2086.7974 | −0.0402 | −19 | 265 | 281 | VHTECCHGDL |
| | | | | | | LECADDR |
| 2124.9875 | 2124.9387 | −0.0488 | −23 | 187 | 205 | AAFTECCQAA |
| | | | | | | DKAACLLPK |
| 2260.0227 | 2259.9587 | −0.064 | −28 | 525 | 543 | EFNAETFTFH |
| | | | | | | ADICTLSEK |
| 2545.1665 | 2545.1023 | −0.0642 | −25 | 525 | 545 | EFNAETFTFH |
| | | | | | | ADICTLSEK |
| | | | | | | ER |
| 2585.1177 | 2585.0464 | −0.0713 | −28 | 265 | 286 | VHTECCHGDL |
| | | | | | | LECADDR |
| | | | | | | ADLAK |
| 2585.1177 | 2585.0464 | −0.0713 | −28 | 265 | 286 | VHTECCHGDL |
| | | | | | | LECADDR |
| | | | | | | ADLAK |
| 2599.2974 | 2599.1162 | −0.1812 | −70 | 414 | 434 | QNCELFEQLG |
| | | | | | | EYKFQNA |
| | | | | | | LLVR |
| 2650.2642 | 2650.1494 | −0.1148 | −43 | 139 | 160 | LVRPEVDVMC |
| | | | | | | TAFHDNE |
| | | | | | | ETFLK |
| 2778.3589 | 2778.2773 | −0.0816 | −29 | 139 | 161 | LVRPEVDVMC |
| | | | | | | TAFHDNE |
| | | | | | | ETFLKK |
| 2794.354 | 2794.2808 | −0.0732 | −26 | 139 | 161 | LVRPEVDVMC |
| | | | | | | TAFHDNE |

3—KH30 (SEQ ID NO. KH30)

| 275/L3 | Instr./Gel Origin |
|---|---|
| [1] Sample Project | |
| 20120229 | Instrument Sample |

| | | |
|---|---|---|
| Accession No. | Protein Name | Name |

| | |
|---|---|
| IPI00219713 | Tax_Id = 9606 Gene_Symbol = |
| | FGG Isoform Gamma-A of |
| | Fibrinogen gamma chain |

Peptide Information

| | | | | Start | End | |
|---|---|---|---|---|---|---|
| Calc. Mass | Obsrv. Mass | ±da | ±ppm | Seq. | Seq. | Sequence |
| 851.441 | 851.4166 | −0.0244 | −29 | 233 | 238 | NWIQYK |
| 1034.5266 | 1034.5286 | 0.002 | 2 | 293 | 301 | VGPEADKYR |
| 1034.5266 | 1034.5286 | 0.002 | 2 | 293 | 301 | VGPEADKYR |
| 1117.5273 | 1117.5242 | −0.0031 | −3 | 274 | 282 | VELEDWNGR |
| 1117.5273 | 1117.5242 | −0.0031 | −3 | 274 | 282 | VELEDWNGR |
| 1134.5137 | 1134.5151 | 0.0014 | 1 | 283 | 292 | TSTADYAMFK |
| 1150.5085 | 1150.5145 | 0.006 | 5 | 283 | 292 | TSTADYAMFK |
| 1194.4878 | 1194.4874 | −0.0004 | 0 | 32 | 40 | DNCCILDER |
| 1194.4878 | 1194.4874 | −0.0004 | 0 | 32 | 40 | DNCCILDER |
| 1293.7566 | 1293.7542 | −0.0024 | −2 | 189 | 199 | QSGLYFIKPLK |
| 1491.7438 | 1491.7455 | 0.0017 | 1 | 122 | 134 | YEASILTHDSS |
| | | | | | | IR |
| 1491.7438 | 1491.7455 | 0.0017 | 1 | 122 | 134 | YEASILTHDSS |
| | | | | | | IR |
| 1513.7281 | 1513.7213 | −0.0068 | −4 | 135 | 146 | YLQEIYNSNN |
| | | | | | | QK |
| 1513.7281 | 1513.7213 | −0.0068 | −4 | 135 | 146 | YLQEIYNSNN |
| | | | | | | QK |
| 1682.9589 | 1682.9558 | −0.0031 | −2 | 259 | 273 | IHLISTQSAIPY |
| | | | | | | ALR |
| 1682.9589 | 1682.9558 | −0.0031 | −2 | 259 | 273 | IHLISTQSAIPY |
| | | | | | | ALR |
| 1893.9131 | 1893.9062 | −0.0069 | −4 | 383 | 399 | ASTPNGYDNG |
| | | | | | | IIWATWK |
| 1893.9131 | 1893.9062 | −0.0069 | −4 | 383 | 399 | ASTPNGYDNG |
| | | | | | | IIWATWK |
| 2011.908 | 2011.9115 | 0.0035 | 2 | 365 | 382 | CHAGHLNGVY |
| | | | | | | YQGGTYSK |
| 2207.0405 | 2207.0403 | −0.0002 | 0 | 239 | 258 | EGFGHLSPTG |
| | | | | | | TTEFWLG |
| | | | | | | NEK |
| 2207.0405 | 2207.0403 | −0.0002 | 0 | 239 | 258 | EGFGHLSPTG |
| | | | | | | TTEFWLG |
| | | | | | | NEK |
| 2233.0229 | 2233.074 | 0.0511 | 23 | 274 | 292 | VELEDWNGRT |
| | | | | | | STADYAM |
| | | | | | | FK |
| 2417.0789 | 2417.1531 | 0.0742 | 31 | 41 | 61 | FGSYCPTTCG |
| | | | | | | IADFLSTY |
| | | | | | | QTK |
| 2520.2651 | 2520.2407 | −0.0244 | −10 | 89 | 111 | AIQLTYNPDES |
| | | | | | | SKPNMID |
| | | | | | | AATLK |
| 2536.26 | 2536.2615 | 0.0015 | 1 | 89 | 111 | AIQLTYNPDE |
| | | | | | | SSKPNMID |
| | | | | | | AATLK |
| 2536.26 | 2536.2615 | 0.0015 | 1 | 89 | 111 | AIQLTYNPDE |
| | | | | | | SSKPNMID |
| | | | | | | AATLK |
| 2768.3342 | 2768.345 | 0.0108 | 4 | 154 | 177 | VAQLEAQCQE |
| | | | | | | PCKDTVQ |
| | | | | | | IHDITGK |
| 2768.3342 | 2768.345 | 0.0108 | 4 | 154 | 177 | VAQLEAQCQE |
| | | | | | | PCKDTVQ |
| | | | | | | IHDITGK |

4—KH31 (SEQ ID NO. KH31)

| 276/L4 | Instr./Gel Origin |
|---|---|
| [1] Sample Project | |
| 20120229 | Instrument Sample |

| | | |
|---|---|---|
| Accession No. | Protein Name | Name |

| | |
|---|---|
| IPI00219713 | Tax_Id = 9606 Gene_Symbol = |
| | FGG Isoform Gamma-A of |
| | Fibrinogen gamma chain |

Peptide Information

| | | | | Start | End | |
|---|---|---|---|---|---|---|
| Calc. Mass | Obsrv. Mass | ±da | ±ppm | Seq. | Seq. | Sequence |
| 851.441 | 851.4302 | −0.0108 | −13 | 233 | 238 | NWIQYK |
| 1034.5266 | 1034.5197 | −0.0069 | −7 | 293 | 301 | VGPEADKYR |
| 1034.5266 | 1034.5197 | −0.0069 | −7 | 293 | 301 | VGPEADKYR |
| 1117.5273 | 1117.5216 | −0.0057 | −5 | 274 | 282 | VELEDWNGR |
| 1117.5273 | 1117.5216 | −0.0057 | −5 | 274 | 282 | VELEDWNGR |
| 1134.5137 | 1134.5092 | −0.0045 | −4 | 283 | 292 | TSTADYAMFK |
| 1150.5085 | 1150.5099 | 0.0014 | 1 | 283 | 292 | TSTADYAMFK |
| 1194.4878 | 1194.4838 | −0.004 | −3 | 32 | 40 | DNCCILDER |
| 1194.4878 | 1194.4838 | −0.004 | −3 | 32 | 40 | DNCCILDER |
| 1293.7566 | 1293.7471 | −0.0095 | −7 | 189 | 199 | QSGLYFIKPLK |
| 1491.7438 | 1491.7361 | −0.0077 | −5 | 122 | 134 | YEASILTHDSSIR |
| 1491.7438 | 1491.7361 | −0.0077 | −5 | 122 | 134 | YEASILTHDSSIR |
| 1513.7281 | 1513.7198 | −0.0083 | −5 | 135 | 146 | YLQEIYNSNNQK |
| 1513.7281 | 1513.7198 | −0.0083 | −5 | 135 | 146 | YLQEIYNSNNQK |
| 1682.9589 | 1682.9517 | −0.0072 | −4 | 259 | 273 | IHLISTQSAIPYA |
| | | | | | | LR |
| 1682.9589 | 1682.9517 | −0.0072 | −4 | 259 | 273 | IHLISTQSAIPYA |
| | | | | | | LR |
| 1893.9131 | 1893.8938 | −0.0193 | −10 | 383 | 399 | ASTPNGYDNGII |
| | | | | | | WATWK |
| 2011.908 | 2011.9026 | −0.0054 | −3 | 365 | 382 | CHAGHLNGVYY |
| | | | | | | QGGTYSK |
| 2207.0405 | 2207.0486 | 0.0081 | 4 | 239 | 258 | EGFGHLSPTGT |
| | | | | | | TEFWLG |
| | | | | | | NEK |
| | | | | | | NEK |
| | | | | | | FKR |
| | | | | | | QTK |
| | 2520.26 | | | | | AATLK |
| | | | | | | AIQLTYNPDE |
| | | | | | | AATLK |
| | | | | | | AATLK |
| | | | | | | WTVFQK |
| | | | | | | IHDITGK |

5—KH32 (SEQ ID NO. KH32)

| | |
|---|---|
| 277/L5 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00220327 | Tax_Id = 9606 Gene_Symbol = |
| | KRT1 Keratin, type II |
| | cytoskeletal 1 |

Peptide Information

| | | | | Start | End | |
|---|---|---|---|---|---|---|
| Calc. Mass | Obsrv. Mass | ±da | ±ppm | Seq. | Seq. | Sequence |
| 1033.516 | 1033.528 | 0.012 | 12 | 484 | 492 | TLLEGEESR |
| 1065.5211 | 1065.5406 | 0.0195 | 18 | 356 | 364 | AQYEDIAQK |
| 1065.5211 | 1065.5406 | 0.0195 | 18 | 356 | 364 | AQYEDIAQK |
| 1265.6372 | 1265.6401 | 0.0029 | 2 | 278 | 288 | TNAENEFVTIK |
| 1302.7012 | 1302.6802 | −0.021 | −16 | 393 | 403 | NSKIEISELNR |
| 1302.7152 | 1302.6802 | −0.035 | −27 | 344 | 355 | SLDLDSIIAEVK |
| 1316.5247 | 1316.5283 | 0.0036 | 3 | 258 | 267 | NMQDMVEDYR |
| 1340.6692 | 1340.6746 | 0.0054 | 4 | 365 | 376 | SKAEAESLYQ |
| | | | | | | SK |
| 1357.6958 | 1357.7135 | 0.0177 | 13 | 444 | 455 | LNDLEDALQQ |
| | | | | | | AK |
| 1357.6958 | 1357.7135 | 0.0177 | 13 | 444 | 455 | LNDLEDALQQ |
| | | | | | | AK |
| 1393.7322 | 1393.739 | 0.0068 | 5 | 278 | 289 | TNAENEFVTIKK |
| 1393.7322 | 1393.739 | 0.0068 | 5 | 278 | 289 | TNAENEFVTIKK |
| 1523.7887 | 1523.7927 | 0.004 | 3 | 461 | 472 | LLRDYQELMN |
| | | | | | | TK |
| 1657.793 | 1657.7776 | −0.0154 | −9 | 13 | 29 | SGGGFSSGS |
| | | | | | | AGIINYQR |
| 1985.8943 | 1985.9266 | 0.0323 | 16 | 252 | 267 | LDSELKNMQD |
| | | | | | | MVEDYR |
| 2286.1248 | 2286.1003 | −0.0245 | −11 | 367 | 386 | AEAESLYQSK |
| | | | | | | YEELQITA |
| | | | | | | GR |
| 3312.3083 | 3312.375 | 0.0667 | 20 | 550 | 588 | GSYGSGGSS |
| | | | | | | YGSGGGS |
| | | | | | | YGSGGGGGG |
| | | | | | | HGSYGSG |
| | | | | | | SSSGGYR |
| 3312.3083 | 3312.375 | 0.0667 | 20 | 550 | 588 | GSYGSGGSS |
| | | | | | | YGSGGGS |
| | | | | | | YGSGGGGGG |
| | | | | | | HGSYGSG |
| | | | | | | SSSGGYR |

6—KH33 (SEQ ID NO. KH33)

| | |
|---|---|
| 278/L6 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00029739 | Tax_Id = 9606 Gene_Symbol = |
| | CFH Isoform 1 of Complement |
| | factor H |

Peptide Information

| | | | | Start | End | |
|---|---|---|---|---|---|---|
| Calc. Mass | Obsrv. Mass | ±da | ±ppm | Seq. | Seq. | Sequence |
| 1063.5895 | 1063.6074 | 0.0179 | 17 | 574 | 582 | IDVHLVPDR |
| 1064.5735 | 1064.5974 | 0.0239 | 22 | 225 | 232 | IIYKENER |
| 1131.5178 | 1131.5262 | 0.0084 | 7 | 770 | 778 | EFDHNSNIR |
| 1133.6466 | 1133.6283 | −0.0183 | −16 | 342 | 351 | RPYFPVAVGK |
| 1141.5605 | 1141.571 | 0.0105 | 9 | 1163 | 1171 | CLHPCVISR |
| 1189.5306 | 1189.551 | 0.0204 | 17 | 897 | 906 | LSYTCEGGFR |
| 1189.5306 | 1189.551 | 0.0204 | 17 | 897 | 906 | LSYTCEGGFR |
| 1194.5386 | 1194.5817 | 0.0431 | 36 | 886 | 896 | SSQESYAHGTK |
| 1213.5531 | 1213.6311 | 0.078 | 64 | 332 | 341 | HGGLYHENMR |
| 1242.542 | 1242.5555 | 0.0135 | 11 | 286 | 295 | TGDEITYQCR |
| 1242.542 | 1242.5555 | 0.0135 | 11 | 286 | 295 | TGDEITYQCR |
| 1245.6045 | 1245.6077 | 0.0032 | 3 | 309 | 319 | CTSTGWIPAPR |
| 1259.6453 | 1259.646 | 0.0007 | 1 | 858 | 867 | WQSIPLCVEK |
| 1262.5835 | 1262.6198 | 0.0363 | 29 | 497 | 507 | DGWSAQPTCIK |
| 1309.6028 | 1309.6433 | 0.0405 | 31 | 508 | 518 | SCDIPVFMNAR |
| 1309.6028 | 1309.6433 | 0.0405 | 31 | 508 | 518 | SCDIPVFMNAR |
| 1311.6362 | 1311.6445 | 0.0083 | 6 | 1193 | 1203 | TGESVEFVCKR |
| 1325.5977 | 1325.6309 | 0.0332 | 25 | 508 | 518 | SCDIPVFMNAR |
| 1325.5977 | 1325.6309 | 0.0332 | 25 | 508 | 518 | SCDIPVFMNAR |
| 1381.678 | 1381.6797 | 0.0017 | 1 | 1172 | 1182 | EIMENYNIALR |
| | 1381.6797 | | | | | |
| 1395.7842 | 1395.7526 | −0.0316 | −23 | 755 | 766 | SSNLIILEEHLK |
| 1527.7625 | 1527.785 | 0.0225 | 15 | 411 | 424 | SIDVACHPGY |
| | | | | | | ALPK |
| 1654.7418 | 1654.7844 | 0.0426 | 26 | 1010 | 1023 | AGEQVTYTCA |
| | | | | | | TYYK |
| 1851.9421 | 1851.947 | 0.0049 | 3 | 1172 | 1186 | EIMENYNIALR |
| | | | | | | WTAK |
| 1851.9421 | 1851.947 | 0.0049 | 3 | 1172 | 1186 | EIMENYNIALR |
| | | | | | | WTAK |
| 1867.828 | 1867.9028 | 0.0748 | 40 | 266 | 281 | SCDNPYIPNG |
| | | | | | | DYSPLR |
| 1867.9371 | 1867.9028 | −0.0343 | −18 | 1172 | 1186 | EIMENYNIALR |
| | | | | | | WTAK |
| 1965.9698 | 1966.0179 | 0.0481 | 24 | 625 | 642 | EQVQSCGPPP |
| | | | | | | ELLNGNV K |
| 2020.9757 | 2020.9901 | 0.0144 | 7 | 1045 | 1062 | DTSCVNPPTV |
| | | | | | | QNAYIVSR |
| 2020.9869 | 2020.9901 | 0.0032 | 2 | 868 | 885 | IPCSQPPQIEH |
| | | | | | | GTINSSR |
| 2057.8811 | 2057.9634 | 0.0823 | 40 | 389 | 404 | CYFPYLENGY |
| | | | | | | NQNYGR |
| 2150.937 | 2150.9851 | 0.0481 | 22 | 128 | 145 | ECDTDGWTN |
| | | | | | | DIPICEVVK |
| 2163.0176 | 2163.0195 | 0.0019 | 1 | 110 | 127 | AVYTCNEGYQ |
| | | | | | | LLGEINYR |
| 2192.0554 | 2192.0154 | −0.04 | −18 | 157 | 175 | IVSSAMEPDR |
| | | | | | | EYHFGQA VR |
| 2208.0503 | 2208.0881 | 0.0378 | 17 | 157 | 175 | IVSSAMEPDR |
| | | | | | | EYHFGQA |
| | | | | | | VR |
| 2259.9976 | 2260.0339 | 0.0363 | 16 | 645 | 662 | TKEEYGHSEV |
| | | | | | | VEYYCNPR |
| 2259.9976 | 2260.0339 | 0.0363 | 16 | 645 | 662 | TKEEYGHSEV |
| | | | | | | VEYYCNPR |

7—KH34 (SEQ ID NO. KH34)

| | |
|---|---|
| 279/L7 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00384853 | Tax_Id = 9606 Gene_Symbol = - |
| | Conserved hypothetical protein |

Peptide Information

| | | | | Start | End | |
|---|---|---|---|---|---|---|
| Calc. Mass | Obsrv. Mass | ±da | ±ppm | Seq. | Seq. | Sequence |
| 1262.6344 | 1262.5671 | −0.0673 | −53 | 100 | 110 | CLVGAQCVLSR |
| 1302.5719 | 1302.6827 | 0.1108 | 85 | 64 | 74 | CSAGAQFVFCR |
| 1320.6324 | 1320.6317 | −0.0007 | −1 | 76 | 88 | SAGAACTQQAL |
| | | | | | | SR |
| 1323.6145 | 1323.6329 | 0.0184 | 14 | 125 | 135 | CTVCTQQALSR |
| 1458.673 | 1458.7278 | 0.0548 | 38 | 64 | 75 | CSAGAQFVFCRR |
| 1851.849 | 1851.9384 | 0.0894 | 48 | 59 | 74 | QAHGRCSAGA |
| | | | | | | QFVFCR |
| 1851.849 | 1851.9384 | 0.0894 | 48 | 59 | 74 | QAHGRCSAGA |
| | | | | | | QFVFCR |

8—KH35 (SEQ ID NO. KH35)

| | |
|---|---|
| 280/L8 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00479708 | Tax_Id = 9606 Gene_Symbol = |
| | IGHM Full-length cDNA clone |
| | CS0DD006YL02 of Neuro- |
| | blastoma of Homo sapiens |

Peptide Information

| | | | | Start | End | |
|---|---|---|---|---|---|---|
| Calc. Mass | Obsrv. Mass | ±da | ±ppm | Seq. | Seq. | Sequence |
| 900.5302 | 900.5189 | −0.0113 | −13 | 35 | 42 | VSVFVPPR |
| 909.4213 | 909.4136 | −0.0077 | −8 | 43 | 50 | DGFFGNPR |
| 909.4213 | 909.4136 | −0.0077 | −8 | 43 | 50 | DGFFGNPR |
| 1029.584 | 1029.5831 | −0.0009 | −1 | 65 | 72 | QIQVSWLR |
| 1029.584 | 1029.5831 | −0.0009 | −1 | 65 | 72 | QIQVSWLR |
| 1249.6359 | 1249.6318 | −0.0041 | −3 | 54 | 64 | LICQATGFSPR |
| 1249.6359 | 1249.6318 | −0.0041 | −3 | 54 | 64 | LICQATGFSPR |
| 1386.8356 | 1386.8224 | −0.0132 | −10 | 22 | 34 | NVPLPVIAELPPK |
| 1464.7628 | 1464.7626 | −0.0002 | 0 | 52 | 64 | SKLICQATGFSPR |
| 1600.7788 | 1600.7754 | −0.0034 | −2 | 299 | 313 | YVTSAPMPEPQ |
| | | | | | | APGR |
| 1600.7788 | 1600.7754 | −0.0034 | −2 | 299 | 313 | YVTSAPMPEPQ |
| | | | | | | APGR |
| 1616.7738 | 1616.7579 | −0.0159 | −10 | 299 | 313 | YVTSAPMPEPQ |
| | | | | | | APGR |
| 1616.7738 | 1616.7579 | −0.0159 | −10 | 299 | 313 | YVTSAPMPEPQ |
| | | | | | | APGR |
| 1646.6938 | 1646.7057 | 0.0119 | 7 | 108 | 120 | ESDWLSQSMFT |
| | | | | | | CR |
| 1646.6938 | 1646.7057 | 0.0119 | 7 | 108 | 120 | ESDWLSQSMFT |
| | | | | | | CR |
| 1662.6887 | 1662.7025 | 0.0138 | 8 | 108 | 120 | ESDWLSQSMFT |
| | | | | | | CR |
| 1662.6887 | 1662.7025 | 0.0138 | 8 | 108 | 120 | ESDWLSQSMFT |
| | | | | | | CR |
| 1716.8627 | 1716.8518 | −0.0109 | −6 | 223 | 237 | FTCTVTHTDLPS |
| | | | | | | PLK |
| 1774.0123 | 1773.9991 | −0.0132 | −7 | 245 | 260 | GVALHRPDV |
| | | | | | | YLLPPAR |
| 1774.0123 | 1773.9991 | −0.0132 | −7 | 245 | 260 | GVALHRPDVY |
| | | | | | | LLPPAR |
| 1790.9337 | 1790.9369 | 0.0032 | 2 | 35 | 50 | VSVFVPPRDG |
| | | | | | | FFGNPR |
| 1931.9669 | 1931.9622 | −0.0047 | −2 | 73 | 91 | EGKQVGSGVT |
| | | | | | | TDQVQAE |
| | | | | | | AK |
| 2453.2129 | 2453.2119 | −0.001 | 0 | 291 | 313 | GQPLSPEKYV |
| | | | | | | TSAPMPE |
| | | | | | | PQAPGR |

9—KH36 (SEQ ID NO. KH36)

| | |
|---|---|
| 281/L9 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00298497 | Tax_Id = 9606 Gene_Symbol = |
| | FGB Fibrinogen beta chain |

Peptide Information

| | | | | Start | | |
|---|---|---|---|---|---|---|
| Calc. Mass | Obsrv. Mass | ±da | ±ppm | Seq. | End Seq. | Sequence |
| 844.4886 | 844.4984 | 0.0098 | 12 | 200 | 206 | SILENLR |
| 886.3876 | 886.3976 | 0.01 | 11 | 472 | 478 | GSWYSMR |
| 980.4432 | 980.4614 | 0.0182 | 19 | 286 | 294 | QDGSVDFGR |
| 1032.5626 | 1032.5872 | 0.0246 | 24 | 484 | 491 | IRPFFPQQ |
| 1032.5626 | 1032.5872 | 0.0246 | 24 | 484 | 491 | IRPFFPQQ |
| 1239.5178 | 1239.558 | 0.0402 | 32 | 427 | 436 | EDGGGWWYNR |
| 1239.5178 | 1239.558 | 0.0402 | 32 | 427 | 436 | EDGGGWWYNR |
| 1275.5601 | 1275.6143 | 0.0542 | 42 | 411 | 421 | DNDGWLTSDPR |
| 1535.7238 | 1535.7637 | 0.0399 | 26 | 354 | 367 | AHYGGFTVQNE |
| | | | | | | ANK |
| 1544.6952 | 1544.7599 | 0.0647 | 42 | 459 | 471 | HGTDDGVVWM |
| | | | | | | NWK |
| 1560.6901 | 1560.7675 | 0.0774 | 50 | 459 | 471 | HGTDDGVVWM |
| | | | | | | NWK |
| 1587.6777 | 1587.7399 | 0.0622 | 39 | 212 | 224 | LESDVSAQMEY |
| | | | | | | CR |
| 1603.6727 | 1603.744 | 0.0713 | 44 | 212 | 224 | LESDVSAQMEY |
| | | | | | | CR |
| 1618.7928 | 1618.8191 | 0.0263 | 16 | 225 | 239 | TPCTVSCNIPVV |
| | | | | | | SGK |
| 1668.7152 | 1668.7885 | 0.0733 | 44 | 446 | 458 | YYWGGQYTWD |
| | | | | | | MAK |
| 1684.7101 | 1684.7656 | 0.0555 | 33 | 446 | 458 | YYWGGQYTWD |
| | | | | | | MAK |
| 1742.734 | 1742.7931 | 0.0591 | 34 | 423 | 436 | QCSKEDGGGW |
| | | | | | | WYNR |
| 1742.734 | 1742.7931 | 0.0591 | 34 | 423 | 436 | QCSKEDGGGW |
| | | | | | | WYNR |
| 1785.7902 | 1785.8638 | 0.0736 | 41 | 314 | 328 | NYCGLPGEYWL |
| | | | | | | GNDK |
| 1951.0032 | 1951.0822 | 0.079 | 40 | 54 | 72 | EEAPSLRPAPP |
| | | | | | | PISGGGYR |
| 1951.0032 | 1951.0822 | 0.079 | 40 | 54 | 72 | EEAPSLRPAP |
| | | | | | | PPISGGGYR |
| 1956.9154 | 1957.0085 | 0.0931 | 48 | 209 | 224 | IQKLESDVSA |
| | | | | | | QMEYCR |
| 1972.9103 | 1973.0043 | 0.094 | 48 | 209 | 224 | IQKLESDVSA |
| | | | | | | QMEYCR |
| 2107.1042 | 2107.2041 | 0.0999 | 47 | 53 | 72 | REEAPSLRPA |
| | | | | | | PPPISGGG YR |
| 2127.083 | 2127.1501 | 0.0671 | 32 | 179 | 196 | HQLYIDETVNS |
| | | | | | | NIPTNLR |
| | 2127.1501 | | | | | |
| 2156.9487 | 2157.0571 | 0.1084 | 50 | 268 | 285 | VYCDMNTENG |
| | | | | | | GWTVIQN R |
| 2156.9487 | 2157.0571 | 0.1084 | 50 | 268 | 285 | VYCDMNTEN |
| | | | | | | GGWTVIQN R |
| 2172.9438 | 2173.0884 | 0.1446 | 67 | 268 | 285 | VYCDMNTEN |
| | | | | | | GGWTVIQN R |
| 2257.0806 | 2257.1973 | 0.1167 | 52 | 248 | 267 | GGETSEMYLI |
| | | | | | | QPDSSVKP |
| | | | | | | YR |
| 2257.0806 | 2257.1973 | 0.1167 | 52 | 248 | 267 | GGETSEMYLI |
| | | | | | | QPDSSVKP |
| | | | | | | YR |
| 2273.0754 | 2273.1951 | 0.1197 | 53 | 248 | 267 | GGETSEMYLI |
| | | | | | | QPDSSVKP |
| | | | | | | YR |
| 2368.168 | 2368.2822 | 0.1142 | 48 | 354 | 374 | AHYGGFTVQN |
| | | | | | | EANKYQI |
| | | | | | | SVNK |
| 2385.1755 | 2385.2944 | 0.1189 | 50 | 247 | 267 | KGGETSEMYL |
| | | | | | | IQPDSSVK |
| | | | | | | PYR |
| 2385.1755 | 2385.2944 | 0.1189 | 50 | 247 | 267 | KGGETSEMYL |
| | | | | | | IQPDSSVK |
| | | | | | | PYR |
| 2401.1704 | 2401.3027 | 0.1323 | 55 | 247 | 267 | KGGETSEMYL |
| | | | | | | IQPDSSVK |
| | | | | | | PYR |
| 2484.1978 | 2484.3455 | 0.1477 | 59 | 314 | 334 | NYCGLPGEY |
| | | | | | | WLGNDKIS |
| | | | | | | QLTR |
| 2548.2205 | 2548.3574 | 0.1369 | 54 | 225 | 246 | TPCTVSCNIPV |
| | | | | | | VSGKECE |
| | | | | | | EIIR |
| 2548.2205 | 2548.3574 | 0.1369 | 54 | 225 | 246 | TPCTVSCNIPV |
| | | | | | | VSGKECE |
| | | | | | | EIIR |

10—KH37 (SEQ ID NO. KH37)

| | |
|---|---|
| 282/L10 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00021841 | Tax_Id = 9606 Gene_Symbol = |
| | APOA1 Apolipoprotein A-I |

Peptide Information

| | Obsrv. | | | Start | End | |
|---|---|---|---|---|---|---|
| Calc. Mass | Mass | ±da | ±ppm | Seq. | Seq. | Sequence |
| 1012.5786 | 1012.5571 | −0.0215 | −21 | 231 | 239 | AKPALEDLR |
| 1031.519 | 1031.5276 | 0.0086 | 8 | 165 | 173 | LSPLGEEMR |
| 1152.6372 | 1152.6154 | −0.0218 | −19 | 156 | 164 | QKLHELQEK |
| 1215.6216 | 1215.6481 | 0.0265 | 22 | 220 | 230 | ATEHLSTLSEK |
| 1230.7092 | 1230.7098 | 0.0006 | 0 | 240 | 250 | QGLLPVLESFK |
| 1235.6882 | 1235.66 | −0.0282 | −23 | 37 | 47 | DLATVYVDVLK |
| 1252.6208 | 1252.6462 | 0.0254 | 20 | 121 | 130 | VQPYLDDFQK |
| 1283.5725 | 1283.597 | 0.0245 | 19 | 132 | 140 | WQEEMELYR |
| 1283.5725 | 1283.597 | 0.0245 | 19 | 132 | 140 | WQEEMELYR |
| 1299.5674 | 1299.6086 | 0.0412 | 32 | 132 | 140 | WQEEMELYR |
| 1301.6484 | 1301.6685 | 0.0201 | 15 | 185 | 195 | THLAPYSDELR |
| 1301.6484 | 1301.6685 | 0.0201 | 15 | 185 | 195 | THLAPYSDELR |
| 1318.642 | 1318.6667 | 0.0247 | 19 | 165 | 175 | LSPLGEEMRDR |
| 1318.642 | 1318.6667 | 0.0247 | 19 | 165 | 175 | LSPLGEEMRDR |
| 1323.621 | 1323.6504 | 0.0294 | 22 | 102 | 112 | ETEGLRQEMSK |
| 1380.7158 | 1380.7335 | 0.0177 | 13 | 121 | 131 | VQPYLDDFQKK |
| 1380.7158 | 1380.7335 | 0.0177 | 13 | 121 | 131 | VQPYLDDFQKK |
| 1386.7152 | 1386.7363 | 0.0211 | 15 | 251 | 262 | VSFLSALEEY |
| | | | | | | TK |
| 1400.6693 | 1400.7068 | 0.0375 | 27 | 52 | 64 | DYVSQFEGSA |
| | | | | | | LGK |
| 1400.6693 | 1400.7068 | 0.0375 | 27 | 52 | 64 | DYVSQFEGSA |
| | | | | | | LGK |
| 1411.6675 | 1411.7057 | 0.0382 | 27 | 131 | 140 | KWQEEMELYR |
| 1427.6624 | 1427.7134 | 0.051 | 36 | 131 | 140 | KWQEEMELYR |
| 1451.7529 | 1451.7086 | −0.0443 | −31 | 119 | 130 | AKVQPYLDDF |
| | | | | | | QK |
| 1462.8516 | 1462.876 | 0.0244 | 17 | 35 | 47 | VKDLATVYVD |
| | | | | | | VLK |
| 1467.7914 | 1467.8289 | 0.0375 | 26 | 143 | 155 | VEPLRAELQE |
| | | | | | | GAR |
| 1467.7914 | 1467.8289 | 0.0375 | 26 | 143 | 155 | VEPLRAELQE |
| | | | | | | GAR |
| 1539.7261 | 1539.7433 | 0.0172 | 11 | 132 | 142 | WQEEMELYR |
| | | | | | | QK |
| 1612.7854 | 1612.8112 | 0.0258 | 16 | 70 | 83 | LLDNWDSVTS |
| | | | | | | TFSK |
| 1650.8698 | 1650.9153 | 0.0455 | 28 | 37 | 51 | DLATVYVDVL |
| | | | | | | KDSGR |
| 1815.8508 | 1815.8988 | 0.048 | 26 | 48 | 64 | DSGRDYVSQ |
| | | | | | | FEGSALGK |
| 1815.8508 | 1815.8988 | 0.048 | 26 | 48 | 64 | DSGRDYVSQ |
| | | | | | | FEGSALGK |
| 1932.9338 | 1933.0148 | 0.081 | 42 | 86 | 101 | EQLGPVTQEF |
| | | | | | | WDNLEK |
| 2202.1189 | 2202.2029 | 0.084 | 38 | 84 | 101 | LREQLGPVTQ |
| | | | | | | EFWDNLEK |

11—KH38 (SEQ ID NO. KH38)

| | |
|---|---|
| 283/L11 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00783987 | Tax_Id = 9606 Gene_Symbol = |
| | C3 Complement C3 (Fragment) |

Peptide Information

| | | | | Start | End | |
|---|---|---|---|---|---|---|
| Calc. Mass | Obsrv. Mass | ±da | ±ppm | Seq. | Seq. | Sequence |
| 842.5345 | 842.4744 | −0.0601 | −71 | 593 | 600 | VVLVAVDK |
| 854.4115 | 854.4087 | −0.0028 | −3 | 585 | 592 | IEGDHGAR |
| 888.4825 | 888.4628 | −0.0197 | −22 | 616 | 622 | IWDVVEK |
| 959.5407 | 959.5392 | −0.0015 | −2 | 1592 | 1599 | EALKLEEK |
| 1042.6255 | 1042.6213 | −0.0042 | −4 | 282 | 290 | ISLPESLKR |
| 1110.6266 | 1110.6171 | −0.0095 | −9 | 306 | 315 | VLLDGVQNPR |
| 1139.5408 | 1139.5358 | −0.005 | −4 | 242 | 249 | FYYIYNEK |
| 1208.6021 | 1208.6031 | 0.001 | 1 | 489 | 497 | YYTYLIMNK |
| 1224.5969 | 1224.5972 | 0.0003 | 0 | 489 | 497 | YYTYLIMNK |
| 1238.7216 | 1238.7168 | −0.0048 | −4 | 305 | 315 | KVLLDGVQNPR |
| 1238.7216 | 1238.7168 | −0.0048 | −4 | 305 | 315 | KVLLDGVQNPR |
| 1345.6998 | 1345.6907 | −0.0091 | −7 | 545 | 556 | EVVADSVWVDVK |
| 1370.7314 | 1370.7313 | −0.0001 | 0 | 137 | 148 | TIYTPGSTVLYR |
| 1370.7314 | 1370.7313 | −0.0001 | 0 | 137 | 148 | TIYTPGSTVLYR |
| 1389.6969 | 1389.6991 | 0.0022 | 2 | 428 | 439 | KQELSEAEQATR |
| 1470.7799 | 1470.776 | −0.0039 | −3 | 291 | 304 | IPIEDGSGEVVL |
| | | | | | | SR |
| 1470.7799 | 1470.776 | −0.0039 | −3 | 291 | 304 | IPIEDGSGEVVL |
| | | | | | | SR |
| 1477.7872 | 1477.7933 | 0.0061 | 4 | 487 | 497 | IRYYTYLIMNK |
| 1511.8217 | 1511.8231 | 0.0014 | 1 | 531 | 544 | LVAYYTLIGASG |
| | | | | | | QR |
| 1511.8217 | 1511.8231 | 0.0014 | 1 | 531 | 544 | LVAYYTLIGASG |
| | | | | | | QR |
| 1598.8749 | 1598.8751 | 0.0002 | 0 | 291 | 305 | IPIEDGSGEVVL |
| | | | | | | SRK |
| 1626.881 | 1626.8781 | −0.0029 | −2 | 290 | 304 | RIPIEDGSGEVV |
| | | | | | | LSR |
| 1639.8724 | 1639.8807 | 0.0083 | 5 | 162 | 176 | TVMVNIENPEGI |
| | | | | | | PVK |
| 1787.9691 | 1787.9709 | 0.0018 | 1 | 344 | 359 | SGIPIVTSPYQIH |
| | | | | | | FTK |
| 1787.9691 | 1787.9709 | 0.0018 | 1 | 344 | 359 | SGIPIVTSPYQIH |
| | | | | | | FTK |
| 1823.0022 | 1823.0173 | 0.0151 | 8 | 306 | 322 | VLLDGVQNPR |
| | | | | | | AEDLVGK |
| 1841.9908 | 1841.8628 | −0.128 | −69 | 1463 | 1478 | VHQYFNVELI |
| | | | | | | QPGAVK |
| 1865.9393 | 1865.9467 | 0.0074 | 4 | 265 | 281 | VEGTAFVIFGI |
| | | | | | | QDGEQR |
| 1872.0338 | 1872.0364 | 0.0026 | 1 | 463 | 478 | TELRPGETLN |
| | | | | | | VNFLLR |
| 1872.0338 | 1872.0364 | 0.0026 | 1 | 463 | 478 | TELRPGETLN |
| | | | | | | VNFLLR |
| 1878.9735 | 1878.9792 | 0.0057 | 3 | 226 | 241 | EYVLPSFEVIV |
| | | | | | | EPTEK |
| 1891.076 | 1891.0774 | 0.0014 | 1 | 409 | 425 | LSINTHPSQKP |
| | | | | | | LSITVR |
| 1891.076 | 1891.0774 | 0.0014 | 1 | 409 | 425 | LSINTHPSQKP |
| | | | | | | LSITVR |
| 1913.0314 | 1913.0513 | 0.0199 | 10 | 1186 | 1203 | SYTVAIAGYAL |
| | | | | | | AQMGRLK |
| 1985.9709 | 1985.9758 | 0.0049 | 2 | 567 | 584 | SGQSEDRQP |
| | | | | | | VPGQQMT |
| | | | | | | LK |
| 1985.9709 | 1985.9758 | 0.0049 | 2 | 567 | 584 | SGQSEDRQP |
| | | | | | | VPGQQMT |
| | | | | | | LK |
| 2001.9658 | 2001.972 | 0.0062 | 3 | 567 | 584 | SGQSEDRQP |
| | | | | | | VPGQQMT |
| | | | | | | LK |
| 2166.0027 | 2166.0095 | 0.0068 | 3 | 208 | 225 | AYYENSPQQV |
| | | | | | | FSTEFEVK |
| 2198.1301 | 2198.1296 | −0.0005 | 0 | 387 | 408 | VPVAVQGEDT |
| | | | | | | VQSLTQG |
| | | | | | | DGVAK |
| 2390.2273 | 2390.239 | 0.0117 | 5 | 545 | 566 | EVVADSVWVD |
| | | | | | | VKDSCVG |
| | | | | | | SLVVK |
| 2444.3071 | 2444.343 | 0.0359 | 15 | 509 | 530 | EPGQDLVVLP |
| | | | | | | LSITTDFIP |
| | | | | | | SFR |
| 2494.1482 | 2494.2024 | 0.0542 | 22 | 634 | 657 | DYAGVFSDAG |
| | | | | | | LTFTSSS |
| | | | | | | GQQTAQR |
| 2578.3083 | 2578.3513 | 0.043 | 17 | 440 | 462 | TMQALPYSTV |
| | | | | | | GNSNNYL |
| | | | | | | HLSVLR |
| 2594.3032 | 2594.3254 | 0.0222 | 9 | 440 | 462 | TMQALPYSTV |
| | | | | | | GNSNNYL |
| | | | | | | HLSVLR |
| 3378.6523 | 3378.7566 | 0.1043 | 31 | 36 | 66 | LESEETMVLE |
| | | | | | | AHDAQGD |
| | | | | | | VPVTVTVHDF |
| | | | | | | PGKK |
| 3537.6187 | 3537.7158 | 0.0971 | 27 | 623 | 657 | ADIGCTPGSG |
| | | | | | | KDYAGVF |
| | | | | | | SDAGLTFTSS |
| | | | | | | SGQQTAQR |

12—KH39 (SEQ ID NO. KH39)

| | |
|---|---|
| 284/L12 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00022434 | Tax_Id = 9606 Gene_Symbol = |
| | ALB Uncharacterized protein |

Peptide Information

| | Obsrv. | | | Start | End | |
|---|---|---|---|---|---|---|
| Calc. Mass | Mass | ±da | ±ppm | Seq. | Seq. | Sequence |
| 875.5098 | 875.4982 | −0.0116 | −13 | 243 | 249 | LSQRFPK |
| 927.4934 | 927.4836 | −0.0098 | −11 | 162 | 168 | YLYEIAR |
| 927.4934 | 927.4836 | −0.0098 | −11 | 162 | 168 | YLYEIAR |
| 960.5625 | 960.5525 | −0.01 | −10 | 427 | 434 | FQNALLVR |
| 1055.5884 | 1055.5841 | −0.0043 | −4 | 161 | 168 | KYLYEIAR |
| | 1055.5841 | | | | | |
| 1074.5426 | 1074.5417 | −0.0009 | −1 | 206 | 214 | LDELRDEGK |
| 1138.498 | 1138.4924 | −0.0056 | −5 | 500 | 508 | CCTESLVNR |
| 1149.615 | 1149.6042 | −0.0108 | −9 | 66 | 75 | LVNEVTEFAK |
| 1149.615 | 1149.6042 | −0.0108 | −9 | 66 | 75 | LVNEVTEFAK |
| 1226.6052 | 1226.5979 | −0.0073 | −6 | 35 | 44 | FKDLGEENFK |
| 1311.7419 | 1311.7471 | 0.0052 | 4 | 362 | 372 | HPDYSVVLLLR |
| 1342.6348 | 1342.6254 | −0.0094 | −7 | 570 | 581 | AVMDDFAAFVEK |
| 1342.6348 | 1342.6254 | −0.0094 | −7 | 570 | 581 | AVMDDFAAFVEK |
| 1358.6298 | 1358.6239 | −0.0059 | −4 | 570 | 581 | AVMDDFAAFVEK |
| 1371.5668 | 1371.5664 | −0.0004 | 0 | 187 | 198 | AAFTECCQAADK |
| 1434.5334 | 1434.5306 | −0.0028 | −2 | 106 | 117 | ETYGEMADCCAK |
| 1443.6421 | 1443.6412 | −0.0009 | −1 | 287 | 298 | YICENQDSISSK |
| 1467.8431 | 1467.8422 | −0.0009 | −1 | 361 | 372 | RHPDYSVVLLLR |
| 1511.8429 | 1511.8425 | −0.0004 | 0 | 439 | 452 | VPQVSTPTLVEV |
| | | | | | | SR |
| 1546.7968 | 1546.7894 | −0.0074 | −5 | 299 | 310 | LKECCEKPLLEK |
| 1552.5978 | 1552.5894 | −0.0084 | −5 | 384 | 396 | CCAAADPHECY |
| | | | | | | AK |
| 1623.7876 | 1623.7841 | −0.0035 | −2 | 348 | 360 | DVFLGMFLYEY |
| | | | | | | AR |
| 1627.6904 | 1627.7179 | 0.0275 | 17 | 585 | 598 | ADDKETCFAEE |
| | | | | | | GQK |
| 1639.9379 | 1639.906 | −0.0319 | −19 | 438 | 452 | KVPQVSTPTLVE |
| | | | | | | VSR |
| 1639.9379 | 1639.906 | −0.0319 | −19 | 438 | 452 | KVPQVSTPTLVE |
| | | | | | | VSR |
| 1657.7527 | 1657.7477 | −0.005 | −3 | 414 | 426 | QNCELFEQLGE |
| | | | | | | YK |
| 1662.7251 | 1662.7285 | 0.0034 | 2 | 185 | 198 | YKAAFTECCQA |
| | | | | | | ADK |
| 1714.7966 | 1714.7916 | −0.005 | −3 | 118 | 130 | QEPERNECFLQ |
| | | | | | | HK |
| 1910.9318 | 1910.9291 | −0.0027 | −1 | 509 | 524 | RPCFSALEVDE |
| | | | | | | TYVPK |
| 1910.9318 | 1910.9291 | −0.0027 | −1 | 509 | 524 | RPCFSALEVDE |
| | | | | | | TYVPK |
| 1932.0372 | 1932.0348 | −0.0024 | −1 | 89 | 105 | SLHTLFGDKLCT |
| | | | | | | VATLR |
| 1996.9294 | 1996.9313 | 0.0019 | 1 | 123 | 138 | NECFLQHKDDN |
| | | | | | | PNLPR |
| 2045.0955 | 2045.0941 | −0.0014 | −1 | 397 | 413 | VFDEFKPLVEEP |
| | | | | | | QNLIK |
| 2045.0955 | 2045.0941 | −0.0014 | −1 | 397 | 413 | VFDEFKPLVEEP |
| | | | | | | QNLIK |
| 2086.8376 | 2086.8457 | 0.0081 | 4 | 265 | 281 | VHTECCHGDLL |
| | | | | | | ECADDR |
| 2124.9875 | 2124.9778 | −0.0097 | −5 | 187 | 205 | AAFTECCQAAD |
| | | | | | | KAACLLPK |
| 2260.0227 | 2260.0229 | 0.0002 | 0 | 525 | 543 | EFNAETFTFHAD |
| | | | | | | ICTLSEK |
| 2545.1665 | 2545.2017 | 0.0352 | 14 | 525 | 545 | EFNAETFTFHAD |
| | | | | | | ICTLSEK |
| | | | | | | ER |
| 2545.1665 | 2545.2017 | 0.0352 | 14 | 525 | 545 | EFNAETFTFH |
| | | | | | | ADICTLSEK |
| | | | | | | ER |
| 2585.1177 | 2585.1587 | 0.041 | 16 | 265 | 286 | VHTECCHGDL |
| | | | | | | LECADDR |
| | 2585.1587 | | | | | ADLAK |
| 2585.1177 | 2585.1587 | 0.041 | 16 | 265 | 286 | VHTECCHGDL |
| | | | | | | LECADDR |
| | | | | | | ADLAK |
| 2599.2974 | 2599.2747 | −0.0227 | −9 | 414 | 434 | QNCELFEQLG |
| | | | | | | EYKFQNA |
| | | | | | | LLVR |
| 2650.2642 | 2650.2732 | 0.009 | 3 | 139 | 160 | LVRPEVDVMC |
| | | | | | | TAFHDNE |
| | | | | | | ETFLK |
| 2778.3589 | 2778.3901 | 0.0312 | 11 | 139 | 161 | LVRPEVDVMC |
| | | | | | | TAFHDNE |
| | | | | | | ETFLKK |
| 2778.3589 | 2778.3901 | 0.0312 | 11 | 139 | 161 | LVRPEVDVMC |
| | | | | | | TAFHDNE |
| | | | | | | ETFLKK |
| 2794.354 | 2794.3987 | 0.0447 | 16 | 139 | 161 | LVRPEVDVMC |
| | | | | | | TAFHDNE |
| | | | | | | ETFLKK |
| 2974.3445 | 2974.377 | 0.0325 | 11 | 311 | 337 | SHCIAEVEND |
| | | | | | | EMPADLPS |
| | | | | | | LAADFVESK |

13—KH40 (SEQ ID NO. KH40)

| | |
|---|---|
| 285/L13 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00298497 | Tax_Id = 9606 Gene_Symbol = |
| | FGB Fibrinogen beta chain |

Peptide Information

| | Obsrv. | | | Start | End | |
|---|---|---|---|---|---|---|
| Calc. Mass | Mass | ±da | ±ppm | Seq. | Seq. | Sequence |
| 844.4886 | 844.4741 | −0.0145 | −17 | 200 | 206 | SILENLR |
| 886.3876 | 886.41 | 0.0224 | 25 | 472 | 478 | GSWYSMR |
| 980.4432 | 980.4583 | 0.0151 | 15 | 286 | 294 | QDGSVD |
| | | | | | | FGR |
| 1032.5626 | 1032.5824 | 0.0198 | 19 | 484 | 491 | IRPFFPQQ |
| 1032.5626 | 1032.5824 | 0.0198 | 19 | 484 | 491 | IRPFFPQQ |
| 1239.5178 | 1239.5336 | 0.0158 | 13 | 427 | 436 | EDGGGW |
| | | | | | | WYNR |
| 1239.5178 | 1239.5336 | 0.0158 | 13 | 427 | 436 | EDGGGW |
| | | | | | | WYNR |
| 1544.6952 | 1544.7151 | 0.0199 | 13 | 459 | 471 | HGTDDGVVW |
| | | | | | | MNWK |
| 1587.6777 | 1587.7048 | 0.0271 | 17 | 212 | 224 | LESDVSAQME |
| | | | | | | YCR |
| 1603.6727 | 1603.703 | 0.0303 | 19 | 212 | 224 | LESDVSAQME |
| | | | | | | YCR |
| 1684.7101 | 1684.7894 | 0.0793 | 47 | 446 | 458 | YYWGGQYTW |
| | | | | | | DMAK |
| 1742.734 | 1742.7664 | 0.0324 | 19 | 423 | 436 | QCSKEDGGG |
| | | | | | | WWYNR |
| 1951.0032 | 1951.0122 | 0.009 | 5 | 54 | 72 | EEAPSLRPAP |
| | | | | | | PPISGGGYR |
| 1951.0032 | 1951.0122 | 0.009 | 5 | 54 | 72 | EEAPSLRPAP |
| | | | | | | PPISGGGYR |
| 1956.9154 | 1956.9503 | 0.0349 | 18 | 209 | 224 | IQKLESDVSA |
| | | | | | | QMEYCR |
| 1972.9103 | 1972.9404 | 0.0301 | 15 | 209 | 224 | IQKLESDVSA |
| | | | | | | QMEYCR |
| 2107.1042 | 2107.1023 | −0.0019 | −1 | 53 | 72 | REEAPSLRPA |
| | | | | | | PPPISGGG |
| | | | | | | YR |
| 2127.083 | 2127.0779 | −0.0051 | −2 | 179 | 196 | HQLYIDETVNS |
| | | | | | | NIPTNLR |
| 2156.9487 | 2157.0083 | 0.0596 | 28 | 268 | 285 | VYCDMNTEN |
| | | | | | | GGWTVIQNR |
| 2156.9487 | 2157.0083 | 0.0596 | 28 | 268 | 285 | VYCDMNTEN |
| | | | | | | GGWTVIQNR |
| 2172.9438 | 2173.03 | 0.0862 | 40 | 268 | 285 | VYCDMNTEN |
| | | | | | | GGWTVIQNR |
| 2257.0806 | 2257.1052 | 0.0246 | 11 | 248 | 267 | GGETSEMYLI |
| | | | | | | QPDSSVKP |
| | | | | | | YR |
| 2273.0754 | 2273.0857 | 0.0103 | 5 | 248 | 267 | GGETSEMYLI |
| | | | | | | QPDSSVKP |
| | | | | | | YR |
| 2368.168 | 2368.1965 | 0.0285 | 12 | 354 | 374 | AHYGGFTVQN |
| | | | | | | EANKYQI |
| | | | | | | SVNK |
| 2385.1755 | 2385.1917 | 0.0162 | 7 | 247 | 267 | KGGETSEMYL |
| | | | | | | IQPDSSVK |
| | | | | | | PYR |
| 2401.1704 | 2401.1926 | 0.0222 | 9 | 247 | 267 | KGGETSEMYL |
| | | | | | | IQPDSSVK |
| | | | | | | PYR |
| 2548.2205 | 2548.196 | −0.0245 | −10 | 225 | 246 | TPCTVSCNIPV |
| | | | | | | VSGKECE |
| | | | | | | EIIR |

14—KH41 (SEQ ID NO. KH41)

| | |
|---|---|
| 287/L15 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00645363 | Tax_Id = 9606 Gene_Symbol = |
| | IGHV4-31; IGHG1 Putative |
| | uncharacterized protein |
| | DKFZp686P15220 |

Peptide Information

| | | | | Start | End | |
|---|---|---|---|---|---|---|
| Calc. Mass | Obsrv. Mass | ±da | ±ppm | Seq. | Seq. | Sequence |
| 838.5032 | 838.5 | −0.0032 | −4 | 352 | 359 | ALPAPIEK |
| 1161.6296 | 1161.6312 | 0.0016 | 1 | 386 | 395 | NQVSLTCLVK |
| 1186.6467 | 1186.642 | −0.0047 | −4 | 147 | 158 | GPSVFPLAPSSK |
| 1286.674 | 1286.6803 | 0.0063 | 5 | 370 | 380 | EPQVYTLPPSR |
| 1304.5828 | 1304.6138 | 0.031 | 24 | 107 | 117 | AEDTALYYCAK |
| 1338.6835 | 1338.6791 | −0.0044 | −3 | 96 | 106 | NSLYLQMNSLR |
| 1677.802 | 1677.8113 | 0.0093 | 6 | 300 | 313 | FNWYVDGVEVH |
| | | | | | | NAK |
| 1677.802 | 1677.8113 | 0.0093 | 6 | 300 | 313 | FNWYVDGVEVH |
| | | | | | | NAK |
| 1724.9078 | 1724.9014 | −0.0064 | −4 | 366 | 380 | GQPREPQVYTL |
| | | | | | | PPSR |
| 1724.9078 | 1724.9014 | −0.0064 | −4 | 366 | 380 | GQPREPQVYTL |
| | | | | | | PPSR |
| 1747.9259 | 1747.865 | −0.0609 | −35 | 381 | 395 | DELTKNQVSLT |
| | | | | | | CLVK |
| 1872.9702 | 1872.9886 | 0.0184 | 10 | 370 | 385 | EPQVYTLPPSR |
| | | | | | | DELTK |
| 1872.9702 | 1872.9886 | 0.0184 | 10 | 370 | 385 | EPQVYTLPPSR |
| | | | | | | DELTK |
| 2139.0276 | 2139.0286 | 0.001 | 0 | 281 | 299 | TPEVTCVVVDV |
| | | | | | | SHEDPE |
| | | | | | | VK |
| 2139.0276 | 2139.0286 | 0.001 | 0 | 281 | 299 | TPEVTCVVVDV |
| | | | | | | SHEDPE |
| | | | | | | VK |
| 2544.1313 | 2544.175 | 0.0437 | 17 | 396 | 417 | GFYPSDIAVEW |
| | | | | | | ESNGQP |
| | | | | | | ENNYK |
| 2544.1313 | 2544.175 | 0.0437 | 17 | 396 | 417 | GFYPSDIAV |
| | | | | | | EWESNGQP |
| | | | | | | ENNYK |

15—KH42 (SEQ ID NO. KH42)

| | |
|---|---|
| 288/L16 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00965713 | Tax_Id = 9606 Gene_Symbol = |
| | FGB fibrinogen beta chain |
| | isoform 2 preproprotein |

Peptide Information

| | Obsrv. | | | Start | End | |
|---|---|---|---|---|---|---|
| Calc. Mass | Mass | ±da | ±ppm | Seq. | Seq. | Sequence |
| 844.4886 | 844.4952 | 0.0066 | 8 | 141 | 147 | SILENLR |
| 886.3876 | 886.4086 | 0.021 | 24 | 413 | 419 | GSWYSMR |
| 980.4432 | 980.4391 | −0.0041 | −4 | 227 | 235 | QDGSVDFGR |
| 980.4432 | 980.4391 | −0.0041 | −4 | 227 | 235 | QDGSVDFGR |
| 1032.5626 | 1032.5549 | −0.0077 | −7 | 425 | 432 | IRPFFPQQ |
| 1032.5626 | 1032.5549 | −0.0077 | −7 | 425 | 432 | IRPFFPQQ |
| 1239.5178 | 1239.5162 | −0.0016 | −1 | 368 | 377 | EDGGGWWYNR |
| 1239.5178 | 1239.5162 | −0.0016 | −1 | 368 | 377 | EDGGGWWYNR |
| 1308.6179 | 1308.6721 | 0.0542 | 41 | 242 | 254 | QGFGNVATNT |
| | | | | | | DGK |
| 1535.7238 | 1535.7338 | 0.01 | 7 | 295 | 308 | AHYGGFTVQN |
| | | | | | | EANK |
| 1587.6777 | 1587.6759 | −0.0018 | −1 | 153 | 165 | LESDVSAQME |
| | | | | | | YCR |
| 1603.6727 | 1603.6925 | 0.0198 | 12 | 153 | 165 | LESDVSAQME |
| | | | | | | YCR |
| 1742.734 | 1742.7518 | 0.0178 | 10 | 364 | 377 | QCSKEDGGG |
| | | | | | | WWYNR |
| 1956.9154 | 1956.9871 | 0.0717 | 37 | 150 | 165 | IQKLESDVSA |
| | | | | | | QMEYCR |
| 2127.083 | 2127.0815 | −0.0015 | −1 | 120 | 137 | HQLYIDETVNS |
| | | | | | | NIPTNLR |
| 2156.9487 | 2156.9648 | 0.0161 | 7 | 209 | 226 | VYCDMNTEN |
| | | | | | | GGWTVIQNR |
| 2172.9438 | 2172.9812 | 0.0374 | 17 | 209 | 226 | VYCDMNTEN |
| | | | | | | GGWTVIQNR |
| 2257.0806 | 2257.0881 | 0.0075 | 3 | 189 | 208 | GGETSEMYLI |
| | | | | | | QPDSSVKP |
| | | | | | | YR |
| 2273.0754 | 2273.1035 | 0.0281 | 12 | 189 | 208 | GGETSEMYLI |
| | | | | | | QPDSSVKP |
| | | | | | | YR |
| 2401.1704 | 2401.1489 | −0.0215 | −9 | 188 | 208 | KGGETSEMYL |
| | | | | | | IQPDSSVK |
| | | | | | | PYR |

16—KH43 (SEQ ID NO. KH43)

| | |
|---|---|
| 289/L17 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00219713 | Tax_Id = 9606 Gene_Symbol = |
| | FGG Isoform Gamma-A of |
| | Fibrinogen gamma chain |

Peptide Information

| | Obsrv. | | | Start | End | |
|---|---|---|---|---|---|---|
| Calc. Mass | Mass | ±da | ±ppm | Seq. | Seq. | Sequence |
| 1034.5266 | 1034.5413 | 0.0147 | 14 | 293 | 301 | VGPEADKYR |
| 1034.5266 | 1034.5413 | 0.0147 | 14 | 293 | 301 | VGPEADKYR |
| 1117.5273 | 1117.5367 | 0.0094 | 8 | 274 | 282 | VELEDWNGR |
| 1117.5273 | 1117.5367 | 0.0094 | 8 | 274 | 282 | VELEDWNGR |
| 1134.5137 | 1134.5328 | 0.0191 | 17 | 283 | 292 | TSTADYAMFK |
| 1150.5085 | 1150.5295 | 0.021 | 18 | 283 | 292 | TSTADYAMFK |
| 1194.4878 | 1194.507 | 0.0192 | 16 | 32 | 40 | DNCCILDER |
| 1194.4878 | 1194.507 | 0.0192 | 16 | 32 | 40 | DNCCILDER |
| 1293.7566 | 1293.766 | 0.0094 | 7 | 189 | 199 | QSGLYFIKPLK |
| 1491.7438 | 1491.7627 | 0.0189 | 13 | 122 | 134 | YEASILTHDSS |
| | | | | | | IR |
| 1491.7438 | 1491.7627 | 0.0189 | 13 | 122 | 134 | YEASILTHDSS |
| | | | | | | IR |
| 1513.7281 | 1513.7418 | 0.0137 | 9 | 135 | 146 | YLQEIYNSNN |
| | | | | | | QK |
| 1513.7281 | 1513.7418 | 0.0137 | 9 | 135 | 146 | YLQEIYNSNN |
| | | | | | | QK |
| 1545.8132 | 1545.8203 | 0.0071 | 5 | 418 | 432 | LTIGEGQQHH |
| | | | | | | LGGAK |
| 1682.9589 | 1682.9622 | 0.0033 | 2 | 259 | 273 | IHLISTQSAIPY |
| | | | | | | ALR |
| 1682.9589 | 1682.9622 | 0.0033 | 2 | 259 | 273 | IHLISTQSAIPY |
| | | | | | | ALR |
| 1893.9131 | 1893.9221 | 0.009 | 5 | 383 | 399 | ASTPNGYDNG |
| | | | | | | IIWATWK |
| 1893.9131 | 1893.9221 | 0.009 | 5 | 383 | 399 | ASTPNGYDNG |
| | | | | | | IIWATWK |
| 2011.908 | 2011.9553 | 0.0473 | 24 | 365 | 382 | CHAGHLNGVY |
| | | | | | | YQGGTYSK |
| 2207.0405 | 2207.0752 | 0.0347 | 16 | 239 | 258 | EGFGHLSPTG |
| | | | | | | TTEFWLG |
| | | | | | | NEK |
| 2207.0405 | 2207.0752 | 0.0347 | 16 | 239 | 258 | EGFGHLSPTG |
| | | | | | | TTEFWLG |
| | | | | | | NEK |
| 2233.0229 | 2233.1147 | 0.0918 | 41 | 274 | 292 | VELEDWNGRT |
| | | | | | | STADYAM |
| | | | | | | FK |
| 2286.2466 | 2286.167 | −0.0796 | −35 | 412 | 432 | IIPFNRLTIGEG |
| | | | | | | QQHHLG |
| | | | | | | GAK |
| 2520.2651 | 2520.2727 | 0.0076 | 3 | 89 | 111 | AIQLTYNPDES |
| | | | | | | SKPNMID |
| | | | | | | AATLK |
| 2520.2651 | 2520.2727 | 0.0076 | 3 | 89 | 111 | AIQLTYNPDES |
| | | | | | | SKPNMID |
| | | | | | | AATLK |
| 2536.26 | 2536.2661 | 0.0061 | 2 | 89 | 111 | AIQLTYNPDES |
| | | | | | | SKPNMID |
| | | | | | | AATLK |
| 2768.3342 | 2768.3794 | 0.0452 | 16 | 154 | 177 | VAQLEAQCQE |
| | | | | | | PCKDTVQ |
| | | | | | | IHDITGK |

17—KH44 (SEQ ID NO. KH44)

| | |
|---|---|
| 290/L18 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00022463 | Tax_Id = 9606 Gene_Symbol = |
| | TF Serotransferrin |

Peptide Information

| | | | | Start | End | |
|---|---|---|---|---|---|---|
| Calc. Mass | Obsrv. Mass | ±da | ±ppm | Seq. | Seq. | Sequence |
| 887.4152 | 887.3967 | −0.0185 | −21 | 468 | 475 | SCHTAVGR |
| 997.4771 | 997.474 | −0.0031 | −3 | 62 | 69 | ASYLDCIR |
| 997.4771 | 997.474 | −0.0031 | −3 | 62 | 69 | ASYLDCIR |
| 1015.5101 | 1015.5061 | −0.004 | −4 | 467 | 475 | KSCHTAVGR |
| 1125.5721 | 1125.5708 | −0.0013 | −1 | 61 | 69 | KASYLDCIR |
| 1125.5721 | 1125.5708 | −0.0013 | −1 | 61 | 69 | KASYLDCIR |
| 1195.5525 | 1195.5483 | −0.0042 | −4 | 123 | 132 | DSGFQMNQLR |
| 1195.5525 | 1195.5483 | −0.0042 | −4 | 123 | 132 | DSGFQMNQLR |
| 1208.5299 | 1208.5386 | 0.0087 | 7 | 38 | 46 | CQSFRDHMK |
| 1211.5474 | 1211.5521 | 0.0047 | 4 | 123 | 132 | DSGFQMNQLR |
| 1211.5474 | 1211.5521 | 0.0047 | 4 | 123 | 132 | DSGFQMNQLR |
| 1224.5249 | 1224.5237 | −0.0012 | −1 | 38 | 46 | CQSFRDHMK |
| 1249.606 | 1249.6061 | 0.0001 | 0 | 454 | 464 | SASDLTWDN |
| | | | | | | LK |
| 1249.606 | 1249.6061 | 0.0001 | 0 | 454 | 464 | SASDLTWDN |
| | | | | | | LK |
| 1273.6536 | 1273.658 | 0.0044 | 3 | 226 | 236 | HSTIFENLANK |
| 1276.6321 | 1276.6476 | 0.0155 | 12 | 300 | 310 | EFQLFSSPH |
| | | | | | | GK |
| 1317.5892 | 1317.5781 | −0.0111 | −8 | 27 | 37 | WCAVSEHEA |
| | | | | | | TK |
| 1323.6475 | 1323.6814 | 0.0339 | 26 | 122 | 132 | KDSGFQMN |
| | | | | | | QLR |
| 1323.7168 | 1323.6814 | −0.0354 | −27 | 316 | 327 | DSAHGFLKV |
| | | | | | | PPR |
| 1339.6423 | 1339.6335 | −0.0088 | −7 | 122 | 132 | KDSGFQMN |
| | | | | | | QLR |
| 1377.7009 | 1377.7019 | 0.001 | 1 | 453 | 464 | KSASDLTWD |
| | | | | | | NLK |
| 1379.6989 | 1379.7036 | 0.0047 | 3 | 213 | 225 | CLKDGAGDV |
| | | | | | | AFVK |
| 1415.72 | 1415.7271 | 0.0071 | 5 | 47 | 60 | SVIPSDGPSV |
| | | | | | | ACVK |
| 1478.7349 | 1478.7434 | 0.0085 | 6 | 332 | 343 | MYLGYEYVT |
| | | | | | | AIR |
| 1491.759 | 1491.7697 | 0.0107 | 7 | 298 | 310 | SKEFQLFSS |
| | | | | | | PHGK |
| 1494.7297 | 1494.7303 | 0.0006 | 0 | 332 | 343 | MYLGYEYVT |
| | | | | | | AIR |
| 1494.7297 | 1494.7303 | 0.0006 | 0 | 332 | 343 | MYLGYEYVT |
| | | | | | | AIR |
| 1521.7367 | 1521.7426 | 0.0059 | 4 | 372 | 384 | LKCDEWSVN |
| | | | | | | SVGK |
| 1539.7108 | 1539.7416 | 0.0308 | 20 | 240 | 251 | DQYELLCLD |
| | | | | | | NTR |
| 1543.8149 | 1543.7887 | −0.0262 | −17 | 47 | 61 | SVIPSDGPSV |
| | | | | | | ACVKK |
| 1629.8159 | 1629.8063 | −0.0096 | −6 | 108 | 121 | EDPQTFYYA |
| | | | | | | VAVVK |
| 1689.849 | 1689.858 | 0.009 | 5 | 259 | 273 | DCHLAQVPS |
| | | | | | | HTVVAR |
| 1725.767 | 1725.7739 | 0.0069 | 4 | 385 | 399 | IECVSAETTE |
| | | | | | | DCIAK |
| 1757.9109 | 1757.8904 | −0.0205 | −12 | 108 | 122 | EDPQTFYYA |
| | | | | | | VAVVKK |
| 1817.8044 | 1817.8302 | 0.0258 | 14 | 347 | 362 | EGTCPEAPT |
| | | | | | | DECKPVK |
| 1881.876 | 1881.8823 | 0.0063 | 3 | 237 | 251 | ADRDQYELL |
| | | | | | | CLDNTR |
| 1881.876 | 1881.8823 | 0.0063 | 3 | 237 | 251 | ADRDQYELL |
| | | | | | | CLDNTR |
| 1939.9292 | 1940.0519 | 0.1227 | 63 | 328 | 343 | MDAKMYLGY |
| | | | | | | EYVTAIR |
| 2171.0955 | 2171.116 | 0.0205 | 9 | 144 | 162 | SAGWNIPIGLL |
| | | | | | | YCDLPEPR |
| 2175.0098 | 2175.0344 | 0.0246 | 11 | 400 | 420 | IMNGEADAMS |
| | | | | | | LDGGFVYI |
| | | | | | | AGK |
| 2191.0046 | 2191.0286 | 0.024 | 11 | 400 | 420 | IMNGEADAMS |
| | | | | | | LDGGFVYI |
| | | | | | | AGK |
| 2201.0325 | 2201.03 | −0.0025 | −1 | 344 | 362 | NLREGTCPEA |
| | | | | | | PTDECKP |
| | | | | | | VK |
| 2233.1248 | 2233.1216 | −0.0032 | −1 | 216 | 236 | DGAGDVAFVK |
| | | | | | | HSTIFENL |
| | | | | | | ANK |
| 2549.293 | 2549.3047 | 0.0117 | 5 | 252 | 273 | KPVDEYKDCH |
| | | | | | | LAQVPSH |
| | | | | | | TVVAR |
| 2549.293 | 2549.3047 | 0.0117 | 5 | 252 | 273 | KPVDEYKDCH |
| | | | | | | LAQVPSH |
| | | | | | | TVVAR |

18—KH45 (SEQ ID NO. KH45)

| | |
|---|---|
| 291/L19 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00023006 | Tax_Id = 9606 Gene_Symbol = |
| | ACTC1 Actin, alpha cardiac |
| | muscle 1 |

Peptide Information

| | |
|---|---|
| IPI00008603 | Tax_Id = 9606 |
| | Gene_Symbol = ACTA2 Actin, |
| | aortic smooth muscle |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.5524 | −0.0148 | −16 | 331 | 338 | IIAPPERK |
| 976.4483 | 976.4329 | −0.0154 | −16 | 21 | 30 | AGFAGDDAPR |
| 1036.6514 | 1036.6401 | −0.0113 | −11 | 329 | 337 | IKIIAPPER |
| 1130.5477 | 1130.532 | −0.0157 | −14 | 199 | 208 | GYSFVTTAER |
| 1130.5477 | 1130.532 | −0.0157 | −14 | 199 | 208 | GYSFVTTAER |
| 1171.5712 | 1171.5664 | −0.0048 | −4 | 42 | 52 | HQGVMVGMGQK |
| 1187.566 | 1187.5728 | 0.0068 | 6 | 42 | 52 | HQGVMVGMGQK |
| 1198.7056 | 1198.6915 | −0.0141 | −12 | 31 | 41 | AVFPSIVGRPR |
| 1198.7056 | 1198.6915 | −0.0141 | −12 | 31 | 41 | AVFPSIVGRPR |
| 1203.5609 | 1203.5902 | 0.0293 | 24 | 42 | 52 | HQGVMVGMGQK |
| 1354.6234 | 1354.6309 | 0.0075 | 6 | 53 | 64 | DSYVGDEAQSKR |
| 1500.7078 | 1500.7155 | 0.0077 | 5 | 362 | 374 | QEYDEAGPSI |
| | | | | | | VHR |
| 1500.7078 | 1500.7155 | 0.0077 | 5 | 362 | 374 | QEYDEAGPSI |
| | | | | | | VHR |
| 1623.8411 | 1623.8591 | 0.018 | 11 | 180 | 193 | LDLAGRDLTD |
| | | | | | | YLMK |
| 1627.8439 | 1627.8619 | 0.018 | 11 | 199 | 212 | GYSFVTTAER |
| | | | | | | EIVR |
| 1639.8361 | 1639.8571 | 0.021 | 13 | 180 | 193 | LDLAGRDLTD |
| | | | | | | YLMK |
| 1790.892 | 1790.9082 | 0.0162 | 9 | 241 | 256 | SYELPDGQVIT |
| | | | | | | IGNER |
| 1790.892 | 1790.9082 | 0.0162 | 9 | 241 | 256 | SYELPDGQVIT |
| | | | | | | IGNER |
| 1956.0437 | 1956.0748 | 0.0311 | 16 | 98 | 115 | VAPEEHPTLLT |
| | | | | | | EAPLNPK |
| 1956.0437 | 1956.0748 | 0.0311 | 16 | 98 | 115 | VAPEEHPTLLT |
| | | | | | | EAPLNPK |
| 2228.0652 | 2228.1257 | 0.0605 | 27 | 294 | 314 | DLYANNVLSG |
| | | | | | | GTTMYPGI |
| | | | | | | ADR |

19—KH46 (SEQ ID NO. KH46)

| | |
|---|---|
| 292/L20 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00021841 | Tax_Id = 9606 Gene_Symbol = |
| | APOA1 Apolipoprotein A-I |
| | Peptide Information |

| | | | | Start | End | |
|---|---|---|---|---|---|---|
| Calc. Mass | Obsrv. Mass | ±da | ±ppm | Seq. | Seq. | Sequence |
| 869.5203 | 869.5239 | 0.0036 | 4 | 141 | 147 | QKVEPLR |
| 873.4424 | 873.4615 | 0.0191 | 22 | 148 | 155 | AELQEGAR |
| 1012.5786 | 1012.5906 | 0.012 | 12 | 231 | 239 | AKPALEDLR |
| 1012.5786 | 1012.5906 | 0.012 | 12 | 231 | 239 | AKPALEDLR |
| 1047.5139 | 1047.5322 | 0.0183 | 17 | 165 | 173 | LSPLGEEMR |
| 1152.6372 | 1152.6506 | 0.0134 | 12 | 156 | 164 | QKLHELQEK |
| 1215.6216 | 1215.6224 | 0.0008 | 1 | 220 | 230 | ATEHLSTLSEK |
| 1252.6208 | 1252.6204 | −0.0004 | 0 | 121 | 130 | VQPYLDDFQK |
| 1283.5725 | 1283.5679 | −0.0046 | −4 | 132 | 140 | WQEEMELYR |
| 1299.5674 | 1299.5654 | −0.002 | −2 | 132 | 140 | WQEEMELYR |
| 1301.6484 | 1301.6364 | −0.012 | −9 | 185 | 195 | THLAPYSDELR |
| 1301.6484 | 1301.6364 | −0.012 | −9 | 185 | 195 | THLAPYSDELR |
| 1307.626 | 1307.5776 | −0.0484 | −37 | 102 | 112 | ETEGLRQEMSK |
| 1318.642 | 1318.6289 | −0.0131 | −10 | 165 | 175 | LSPLGEEMRDR |
| 1318.642 | 1318.6289 | −0.0131 | −10 | 165 | 175 | LSPLGEEMRDR |
| 1323.621 | 1323.6036 | −0.0174 | −13 | 102 | 112 | ETEGLRQEMSK |
| 1380.7158 | 1380.6981 | −0.0177 | −13 | 121 | 131 | VQPYLDDFQKK |
| 1380.7158 | 1380.6981 | −0.0177 | −13 | 121 | 131 | VQPYLDDFQKK |
| 1400.6693 | 1400.6514 | −0.0179 | −13 | 52 | 64 | DYVSQFEGSA |
| | | | | | | LGK |
| 1427.6624 | 1427.649 | −0.0134 | −9 | 131 | 140 | KWQEEMELYR |
| 1462.8516 | 1462.7736 | −0.078 | −53 | 35 | 47 | VKDLATVYVD |
| | | | | | | VLK |
| 1467.7914 | 1467.7683 | −0.0231 | −16 | 143 | 155 | VEPLRAELQE |
| | | | | | | GAR |
| 1467.7914 | 1467.7683 | −0.0231 | −16 | 143 | 155 | VEPLRAELQE |
| | | | | | | GAR |
| 1585.8081 | 1585.7871 | −0.021 | −13 | 185 | 197 | THLAPYSDEL |
| | | | | | | RQR |
| 1612.7854 | 1612.7574 | −0.028 | −17 | 70 | 83 | LLDNWDSVTS |
| | | | | | | TFSK |
| 1650.8698 | 1650.8358 | −0.034 | −21 | 37 | 51 | DLATVYVDVL |
| | | | | | | KDSGR |
| 1815.8508 | 1815.8008 | −0.05 | −28 | 48 | 64 | DSGRDYVSQ |
| | | | | | | FEGSALGK |
| 1815.8508 | 1815.8008 | −0.05 | −28 | 48 | 64 | DSGRDYVSQ |
| | | | | | | FEGSALGK |
| 2202.1189 | 2202.0535 | −0.0654 | −30 | 84 | 101 | LREQLGPVTQ |
| | | | | | | EFWDNLE |

20—KH47 (SEQ ID NO. KH47)

| | |
|---|---|
| 293/L21 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00023006 | Tax_Id = 9606 Gene_Symbol = |
| | ACTC1 Actin, alpha cardiac |
| | muscle 1 |

Protein Group

| | |
|---|---|
| IPI00008603 | Tax_Id = 9606 |
| | Gene_Symbol = ACTA2 Actin, |
| | aortic smooth muscle |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.5429 | −0.0243 | −26 | 331 | 338 | IIAPPERK |
| 976.4483 | 976.4276 | −0.0207 | −21 | 21 | 30 | AGFAGDDAPR |
| 1036.6514 | 1036.6403 | −0.0111 | −11 | 329 | 337 | IKIIAPPER |
| 1130.5477 | 1130.5374 | −0.0103 | −9 | 199 | 208 | GYSFVTTAER |
| 1130.5477 | 1130.5374 | −0.0103 | −9 | 199 | 208 | GYSFVTTAER |
| 1198.7056 | 1198.7004 | −0.0052 | −4 | 31 | 41 | AVFPSIVGRPR |
| 1198.7056 | 1198.7004 | −0.0052 | −4 | 31 | 41 | AVFPSIVGRPR |
| 1203.5609 | 1203.5531 | −0.0078 | −6 | 42 | 52 | HQGVMVGMGQK |
| 1354.6234 | 1354.6332 | 0.0098 | 7 | 53 | 64 | DSYVGDEAQSKR |
| 1500.7078 | 1500.7264 | 0.0186 | 12 | 362 | 374 | QEYDEAGPSIV |
| | | | | | | HR |

21—KH48 (SEQ ID NO. KH48)

| | |
|---|---|
| 294/L22 | Instr./Gel Origin |
| [1] Sample Project | |
| 20120229 | |
| | Instrument Sample |

| Accession No. | Protein Name | Name |
|---|---|---|
| IPI00784842 | Tax_Id = 9606 Gene_Symbol = | |
| | IGHV4-31; IGHG1 Putative | |
| | uncharacterized protein | |
| | DKFZp686G11190 | |

Peptide Information

| | | | | Start | End | |
|---|---|---|---|---|---|---|
| Calc. Mass | Obsrv. Mass | ±da | ±ppm | Seq. | Seq. | Sequence |
| 838.5032 | 838.4819 | −0.0213 | −25 | 355 | 362 | ALPAPIEK |
| 1161.6296 | 1161.6184 | −0.0112 | −10 | 389 | 398 | NQVSLTCLVK |
| 1286.674 | 1286.6669 | −0.0071 | −6 | 373 | 383 | EPQVYTLPPSR |
| 1286.674 | 1286.6669 | −0.0071 | −6 | 373 | 383 | EPQVYTLPPSR |
| 1677.802 | 1677.7974 | −0.0046 | −3 | 303 | 316 | FNWYVDGVEVH |
| | | | | | | NAK |
| 1677.802 | 1677.7974 | −0.0046 | −3 | 303 | 316 | FNWYVDGVE |
| | | | | | | VHNAK |
| 1724.9078 | 1724.8474 | −0.0604 | −35 | 369 | 383 | GQPREPQVY |
| | | | | | | TLPPSR |
| 1872.9702 | 1872.979 | 0.0088 | 5 | 373 | 388 | EPQVYTLPPS |
| | | | | | | RDELTK |
| 1872.9702 | 1872.979 | 0.0088 | 5 | 373 | 388 | EPQVYTLPPS |
| | | | | | | RDELTK |
| 2139.0276 | 2139.0415 | 0.0139 | 6 | 284 | 302 | TPEVTCVVVD |
| | | | | | | VSHEDPE |
| | | | | | | VK |
| 2139.0276 | 2139.0415 | 0.0139 | 6 | 284 | 302 | TPEVTCVVVD |
| | | | | | | VSHEDPE |
| | | | | | | VK |
| 2510.282 | 2510.2266 | −0.0554 | −22 | 85 | 106 | GRFTISGDIST |
| | | | | | | NTLYLQM |
| | | | | | | HSLR |
| 2544.1313 | 2544.2078 | 0.0765 | 30 | 399 | 420 | GFYPSDIAVE |
| | | | | | | WESNGQP |
| | | | | | | ENNYK |
| 2544.1313 | 2544.2078 | 0.0765 | 30 | 399 | 420 | GFYPSDIAVE |
| | | | | | | WESNGQP |
| | | | | | | ENNYK |

22—KH49 (SEQ ID NO. KH49)

| | |
|---|---|
| 295/L23 | Instr./Gel Origin |
| [1] Sample Project | |
| 20120229 | |
| | Instrument Sample |

| | | |
|---|---|---|
| Accession No. | Protein Name | Name |

| | |
|---|---|
| IPI00930226 | Tax_Id = 9606 Gene_Symbol = |
| | ACTG1 cDNA FLJ57283, highly |
| | similar to Actin, cytoplasmic 2 |

Peptide Information

| | | | | Start | End | |
|---|---|---|---|---|---|---|
| Calc. Mass | Obsrv. Mass | ±da | ±ppm | Seq. | Seq. | Sequence |
| 923.5672 | 923.568 | 0.0008 | 1 | 310 | 317 | IIAPPERK |
| 976.4483 | 976.4448 | −0.0035 | −4 | 19 | 28 | AGFAGDDAPR |
| 976.4483 | 976.4448 | −0.0035 | −4 | 19 | 28 | AGFAGDDAPR |
| 998.4863 | 998.47 | −0.0163 | −16 | 165 | 172 | DLTDYLMK |
| 1036.6514 | 1036.6456 | −0.0058 | −6 | 308 | 316 | IKIIAPPER |
| 1132.527 | 1132.5206 | −0.0064 | −6 | 178 | 187 | GYSFTTTAER |
| 1132.527 | 1132.5206 | −0.0064 | −6 | 178 | 187 | GYSFTTTAER |
| 1171.5712 | 1171.5726 | 0.0014 | 1 | 40 | 50 | HQGVMVGMGQK |
| 1187.566 | 1187.5695 | 0.0035 | 3 | 40 | 50 | HQGVMVGMGQK |
| 1198.7056 | 1198.6975 | −0.0081 | −7 | 29 | 39 | AVFPSIVGRPR |
| 1198.7056 | 1198.6975 | −0.0081 | −7 | 29 | 39 | AVFPSIVGRPR |
| 1203.5609 | 1203.5579 | −0.003 | −2 | 40 | 50 | HQGVMVGMGQK |
| 1354.6234 | 1354.6196 | −0.0038 | −3 | 51 | 62 | DSYVGDEAQSKR |
| 1515.7491 | 1515.7416 | −0.0075 | −5 | 85 | 95 | IWHHTFYNELR |
| 1516.7028 | 1516.6996 | −0.0032 | −2 | 341 | 353 | QEYDESGPSI |
| | | | | | | VHR |
| 1516.7028 | 1516.6996 | −0.0032 | −2 | 341 | 353 | QEYDESGPSI |
| | | | | | | VHR |
| 1623.8411 | 1623.8331 | −0.008 | −5 | 159 | 172 | LDLAGRDLTD |
| | | | | | | YLMK |
| 1629.8231 | 1629.8138 | −0.0093 | −6 | 178 | 191 | GYSFTTTAER |
| | | | | | | EIVR |
| 1639.8361 | 1639.8345 | −0.0016 | −1 | 159 | 172 | LDLAGRDLTD |
| | | | | | | YLMK |
| 1644.7976 | 1644.7946 | −0.003 | −2 | 341 | 354 | QEYDESGPSI |
| | | | | | | VHRK |
| 1790.892 | 1790.885 | −0.007 | −4 | 220 | 235 | SYELPDGQVI |
| | | | | | | TIGNER |
| 1790.892 | 1790.885 | −0.007 | −4 | 220 | 235 | SYELPDGQVI |
| | | | | | | TIGNER |
| 1954.0645 | 1954.0524 | −0.0121 | −6 | 96 | 113 | VAPEEHPVLL |
| | | | | | | TEAPLNPK |
| 1954.0645 | 1954.0524 | −0.0121 | −6 | 96 | 113 | VAPEEHPVLL |
| | | | | | | TEAPLNPK |
| 2215.0701 | 2215.0815 | 0.0114 | 5 | 273 | 293 | DLYANTVLSG |
| | | | | | | GTTMYPGI |
| | | | | | | ADR |
| 2215.0701 | 2215.0815 | 0.0114 | 5 | 273 | 293 | DLYANTVLSG |
| | | | | | | GTTMYPGI |
| | | | | | | ADR |
| 2231.0649 | 2231.0654 | 0.0005 | 0 | 273 | 293 | DLYANTVLSG |
| | | | | | | GTTMYPGI |
| | | | | | | ADR |
| 2343.165 | 2343.1611 | −0.0039 | −2 | 272 | 293 | KDLYANTVLS |
| | | | | | | GGTTMYP |
| | | | | | | GIADR |

23—KH50 (SEQ ID NO. KH50)

| | |
|---|---|
| 296/L24 | Instr./Gel Origin |
| [1] Sample Project | |
| 20120229 | |
| | Instrument Sample |

| Accession No. | Protein Name | Name |
|---|---|---|
| IPI00022371 | Tax_Id = 9606 | |
| | Gene_Symbol = HRG Histidine- | |
| | rich glycoprotein | |

Peptide Information

| | Obsrv. | | | Start | End | |
|---|---|---|---|---|---|---|
| Calc. Mass | Mass | ±da | ±ppm | Seq. | Seq. | Sequence |
| 942.5731 | 942.5751 | 0.002 | 2 | 34 | 41 | ALDLINKR |
| 1108.5131 | 1108.5219 | 0.0088 | 8 | 112 | 120 | HSHESQDLR |
| 1124.6099 | 1124.6121 | 0.0022 | 2 | 44 | 52 | DGYLFQLLR |
| 1124.6099 | 1124.6121 | 0.0022 | 2 | 44 | 52 | DGYLFQLLR |
| 1133.531 | 1133.53 | −0.001 | −1 | 211 | 219 | HPNVFGFCR |
| 1133.531 | 1133.53 | −0.001 | −1 | 211 | 219 | HPNVFGFCR |
| 1280.7111 | 1280.7106 | −0.0005 | 0 | 43 | 52 | RDGYLFQLLR |
| 1286.6886 | 1286.6846 | −0.004 | −3 | 96 | 106 | RPSEIVIGQCK |
| 1286.6886 | 1286.6846 | −0.004 | −3 | 96 | 106 | RPSEIVIGQCK |
| | 1286.6846 | | | | | |
| 1438.5692 | 1438.5994 | 0.0302 | 21 | 85 | 95 | YWNDCEPPDSR |
| 1490.6912 | 1490.6945 | 0.0033 | 2 | 188 | 201 | GGEGTGYFVDFSVR |
| 1490.6912 | 1490.6945 | 0.0033 | 2 | 188 | 201 | GGEGTGYFVDFSVR |
| 1520.6653 | 1520.6693 | 0.004 | 3 | 165 | 176 | YKEENDDFASFR |
| 1520.6653 | 1520.6693 | 0.004 | 3 | 165 | 176 | YKEENDDFASFR |
| 1566.6642 | 1566.6665 | 0.0023 | 1 | 84 | 95 | KYWNDCEPPDSR |
| 1566.6642 | 1566.6665 | 0.0023 | 1 | 84 | 95 | KYWNDCEPPDSR |
| 1682.7909 | 1682.7927 | 0.0018 | 1 | 140 | 153 | DSPVLIDFFEDTER |
| 1682.7909 | 1682.7927 | 0.0018 | 1 | 140 | 153 | DSPVLIDFFEDTER |
| 1745.8606 | 1745.8588 | −0.0018 | −1 | 186 | 201 | VRGGEGTGYFVDFSVR |
| 1745.8606 | 1745.8588 | −0.0018 | −1 | 186 | 201 | VRGGEGTGYFVDFSVR |
| 1824.8903 | 1824.8805 | −0.0098 | −5 | 220 | 235 | ADLFYDVEALDLESPK |
| 2001.9553 | 2001.9672 | 0.0119 | 6 | 140 | 155 | DSPVLIDFFEDTERYR |

24—KH51 (SEQ ID NO. KH51)

| | |
|---|---|
| 299/M1 | Instr./Gel Origin |
| [1] Sample Project | |
| 20120229 | |
| | Instrument Sample |

| Accession No. | Protein Name | Name |
|---|---|---|
| IPI00022371 | Tax_Id = 9606 Gene_Symbol = | |
| | HRG Histidine-rich glycoprotein | |

Peptide Information

| | | | | Start | End | |
|---|---|---|---|---|---|---|
| Calc. Mass | Obsrv. Mass | ±da | ±ppm | Seq. | Seq. | Sequence |
| 942.5731 | 942.5731 | 0 | 0 | 34 | 41 | ALDLINKR |
| 1108.5131 | 1108.5183 | 0.0052 | 5 | 112 | 120 | HSHESQDLR |
| 1124.6099 | 1124.6101 | 0.0002 | 0 | 44 | 52 | DGYLFQLLR |
| 1124.6099 | 1124.6101 | 0.0002 | 0 | 44 | 52 | DGYLFQLLR |
| 1133.531 | 1133.5333 | 0.0023 | 2 | 211 | 219 | HPNVFGFCR |
| 1133.531 | 1133.5333 | 0.0023 | 2 | 211 | 219 | HPNVFGFCR |
| 1280.7111 | 1280.7091 | −0.002 | −2 | 43 | 52 | RDGYLFQLLR |
| 1286.6886 | 1286.6833 | −0.0053 | −4 | 96 | 106 | RPSEIVIGQCK |
| 1286.6886 | 1286.6833 | −0.0053 | −4 | 96 | 106 | RPSEIVIGQCK |
| 1438.5692 | 1438.5841 | 0.0149 | 10 | 85 | 95 | YWNDCEPPDSR |
| 1490.6912 | 1490.6917 | 0.0005 | 0 | 188 | 201 | GGEGTGYFVDF |
| | | | | | | SVR |
| 1490.6912 | 1490.6917 | 0.0005 | 0 | 188 | 201 | GGEGTGYFVDF |
| | | | | | | SVR |
| 1520.6653 | 1520.6692 | 0.0039 | 3 | 165 | 176 | YKEENDDFASFR |
| 1520.6653 | 1520.6692 | 0.0039 | 3 | 165 | 176 | YKEENDDFASFR |
| 1566.6642 | 1566.6675 | 0.0033 | 2 | 84 | 95 | KYWNDCEPPDSR |
| 1566.6642 | 1566.6675 | 0.0033 | 2 | 84 | 95 | KYWNDCEPPDSR |
| 1682.7909 | 1682.7971 | 0.0062 | 4 | 140 | 153 | DSPVLIDFFEDT |
| | | | | | | ER |
| 1682.7909 | 1682.7971 | 0.0062 | 4 | 140 | 153 | DSPVLIDFFEDT |
| | | | | | | ER |
| 1745.8606 | 1745.8633 | 0.0027 | 2 | 186 | 201 | VRGGEGTGYFV |
| | | | | | | DFSVR |
| 1745.8606 | 1745.8633 | 0.0027 | 2 | 186 | 201 | VRGGEGTGYFV |
| | | | | | | DFSVR |
| 1824.8903 | 1824.8855 | −0.0048 | −3 | 220 | 235 | ADLFYDVEALDL |
| | | | | | | ESPK |
| 1824.8903 | 1824.8855 | −0.0048 | −3 | 220 | 235 | ADLFYDVEALDL |
| | | | | | | ESPK |
| 2001.9553 | 2001.99 | 0.0347 | 17 | 140 | 155 | DSPVLIDFFEDT |
| | | | | | | ERYR |

25—KH52 (SEQ ID NO. KH52)

| | |
|---|---|
| 300/M2 | Instr./Gel Origin |
| [1] Sample Project | |
| 20120229 | |
| | Instrument Sample |

| Accession No. | Protein Name | Name |
|---|---|---|
| IPI00878282 | Tax_Id = 9606 Gene_Symbol = | |
| | ALB 23 kDa protein | |

Peptide Information

| | | | | Start | End | |
|---|---|---|---|---|---|---|
| Calc. Mass | Obsrv. Mass | ±da | ±ppm | Seq. | Seq. | Sequence |
| 927.4934 | 927.4923 | −0.0011 | −1 | 164 | 170 | YLYEIAR |
| 927.4934 | 927.4923 | −0.0011 | −1 | 164 | 170 | YLYEIAR |
| 933.5186 | 933.5193 | 0.0007 | 1 | 100 | 107 | LCTVATLR |
| 940.4482 | 940.4518 | 0.0036 | 4 | 133 | 140 | DDNPNLPR |
| | 940.4518 | | | | | |
| 1055.5884 | 1055.5844 | −0.004 | −4 | 163 | 170 | KYLYEIAR |
| 1083.5946 | 1083.5922 | −0.0024 | −2 | 164 | 171 | YLYEIARR |
| 1149.615 | 1149.5994 | −0.0156 | −14 | 68 | 77 | LVNEVTEFAK |
| 1149.615 | 1149.5994 | −0.0156 | −14 | 68 | 77 | LVNEVTEFAK |
| 1226.6052 | 1226.5889 | −0.0163 | −13 | 37 | 46 | FKDLGEENFK |
| 1226.6052 | 1226.5889 | −0.0163 | −13 | 37 | 46 | FKDLGEENFK |
| 1434.5334 | 1434.5005 | −0.0329 | −23 | 108 | 119 | ETYGEMADCC |
| | | | | | | AK |
| 1434.5334 | 1434.5005 | −0.0329 | −23 | 108 | 119 | ETYGEMADCC |
| | | | | | | AK |
| 1450.5283 | 1450.5132 | −0.0151 | −10 | 108 | 119 | ETYGEMADCC |
| | | | | | | AK |
| 1498.5785 | 1498.5447 | −0.0338 | −23 | 78 | 90 | TCVADESAEN |
| | | | | | | CDK |
| 1714.7966 | 1714.7405 | −0.0561 | −33 | 120 | 132 | QEPERNECFL |
| | | | | | | QHK |
| 1714.7966 | 1714.7405 | −0.0561 | −33 | 120 | 132 | QEPERNECFL |
| | | | | | | QHK |
| 1932.0372 | 1931.9761 | −0.0611 | −32 | 91 | 107 | SLHTLFGDKL |
| | | | | | | CTVATLR |
| 1996.9294 | 1996.8643 | −0.0651 | −33 | 125 | 140 | NECFLQHKDD |
| | | | | | | NPNLPR |
| 1996.9294 | 1996.8643 | −0.0651 | −33 | 125 | 140 | NECFLQHKDD |
| | | | | | | NPNLPR |
| 2497.0969 | 2497.0144 | −0.0825 | −33 | 78 | 99 | TCVADESAEN |
| | | | | | | CDKSLHTL |
| 2650.2642 | 2650.1545 | −0.1097 | −41 | 141 | 162 | FGDK |
| | | | | | | LVRPEVDVMC |
| | | | | | | TAFHDNE |
| | | | | | | ETFLK |
| 2778.3589 | 2778.2568 | −0.1021 | −37 | 141 | 163 | LVRPEVDVMC |
| | | | | | | TAFHDNE |
| | | | | | | ETFLKK |
| 2778.3589 | 2778.2568 | −0.1021 | −37 | 141 | 163 | LVRPEVDVMC |
| | | | | | | TAFHDNE |
| | | | | | | ETFLKK |
| 2794.354 | 2794.2327 | −0.1213 | −43 | 141 | 163 | LVRPEVDVMC |
| | | | | | | TAFHDNE |
| | | | | | | ETFLKK |

In addition to the 52 KH discovered proteins, the inventor has found 3 more new found proteins. 1 in AFCC® (From fraction IV) and 2 in Transferrin with their sequence as follows:

| | | | |
|---|---|---|---|
| Gel Idx/Pos | | 102/E1 | Instr./Gel Origin |
| Plate [#] Name | | [1] Sample Project | Instrument Sample |
| | | 20120802 | Name |
| Rank | Accession No. | Protein Name | |

| | |
|---|---|
| gi\|194380034 | unnamed protein product [Homo sapiens] |
| Protein Group | |
| gi\|6650772 | PRO1400 [Homo sapiens] |
| | Project\Project sample_20120802\ |
| | P12139-2fncbi |

Peptide Information

| | | | | Start | End | |
|---|---|---|---|---|---|---|
| Calc. Mass | Obsrv. Mass | ±da | ±ppm | Seq. | Seq. | Sequence |
| 964.5323 | 964.5747 | 0.0424 | 44 | 474 | 482 | APNHAVVTR |
| 1000.4985 | 1000.5438 | 0.0453 | 45 | 542 | 549 | YLGEEYVK |
| 1195.5426 | 1195.6343 | 0.0917 | 77 | 236 | 244 | WCALSHHER |
| 1249.606 | 1249.7004 | 0.0944 | 76 | 327 | 337 | SASDLTWDNLK |
| 1283.5692 | 1283.6665 | 0.0973 | 76 | 404 | 414 | EGYYGYTGAFR |
| 1283.5692 | 1283.6665 | 0.0973 | 76 | 404 | 414 | EGYYGYTGAFR |
| 1323.7168 | 1323.8091 | 0.0923 | 70 | 189 | 200 | DSAHGFLKVPPR |
| 1476.7516 | 1476.8101 | 0.0585 | 40 | 294 | 306 | CGLVPVLAENYNK |
| 1478.7349 | 1478.8625 | 0.1276 | 86 | 205 | 216 | MYLGYEYVTAIR |
| 1478.7349 | 1478.8625 | 0.1276 | 86 | 205 | 216 | MYLGYEYVTAIR |
| 1491.759 | 1491.8854 | 0.1264 | 85 | 171 | 183 | SKEFQLFSSPHGK |
| 1494.7297 | 1494.8582 | 0.1285 | 86 | 205 | 216 | MYLGYEYVTAIR |
| 1494.7297 | 1494.8582 | 0.1285 | 86 | 205 | 216 | MYLGYEYVTAIR |
| 1531.688 | 1531.8146 | 0.1266 | 83 | 557 | 569 | CSTSSLLEACTFR |
| 1531.688 | 1531.8146 | 0.1266 | 83 | 557 | 569 | CSTSSLLEACTFR |
| 1565.7992 | 1565.9364 | 0.1372 | 88 | 520 | 532 | DLLFRDDTVCLAK |
| 1565.7992 | 1565.9364 | 0.1372 | 88 | 520 | 532 | DLLFRDDTVCLAK |
| 1577.8145 | 1577.8724 | 0.0579 | 37 | 349 | 362 | TAGWNIPMGLLYNK |
| 1586.7744 | 1586.9088 | 0.1344 | 85 | 461 | 473 | KPVEEYANCHLAR |
| 1659.783 | 1659.9326 | 0.1496 | 90 | 556 | 569 | KCSTSSLLEACTFR |
| 1689.849 | 1690.0056 | 0.1566 | 93 | 132 | 146 | DCHLAQVPSHTVVAR |
| 1705.7527 | 1705.9202 | 0.1675 | 98 | 368 | 382 | FDEFFSEGCAPGSKK |
| 1881.876 | 1882.0433 | 0.1673 | 89 | 110 | 124 | ADRDQYELLCLDNTR |
| 1952.9382 | 1953.1014 | 0.1632 | 84 | 445 | 460 | NLNEKDYELLCLDGTR |
| 1952.9382 | 1953.1014 | 0.1632 | 84 | 445 | 460 | NLNEKDYELLCLDGTR |
| 1974.9781 | 1975.1069 | 0.1288 | 65 | 427 | 444 | HQTVPQNTGGKNPDPW |
| | | | | | | AK |
| 2159.0149 | 2159.1621 | 0.1472 | 68 | 273 | 293 | IMNGEADAMSLDGGFVYI |
| | | | | | | AGK |
| 2171.0955 | 2171.3062 | 0.2107 | 97 | 17 | 35 | SAGWNIPIGLLYCDLPEPR |
| 2175.0098 | 2175.0962 | 0.0864 | 40 | 273 | 293 | IMNGEADAMSLDGGFVYI |

| | |
|---|---|
| Project\Project | 2175.096 |
| sample_20120802\P12139- | 2 |
| 2fncbi | |

| | | | | | | |
|---|---|---|---|---|---|---|
| | | | | | | AGK |
| 2258.1321 | 2258.1755 | 0.0434 | 19 | 349 | 367 | TAGWNIPMGLLYNKIN |
| | | | | | | HCR |
| 2549.293 | 2549.4858 | 0.1928 | 76 | 125 | 146 | KPVDEYKDCHLAQVP |
| | | | | | | SH |
| | | | | | | TVVAR |
| 2549.293 | 2549.4858 | 0.1928 | 76 | 125 | 146 | KPVDEYKDCHLAQVP |
| | | | | | | SH |
| | | | | | | TVVAR |
| 2971.3171 | 2971.553 | 0.2359 | 79 | 389 | 414 | LCMGSGLNLCEPNNK |
| | | | | | | EG |
| | | | | | | YYGYTGAFR |
| 2971.3171 | 2971.553 | 0.2359 | 79 | 389 | 414 | LCMGSGLNLCEPNNK |
| | | | | | | EG |
| | | | | | | YYGYTGAFR |
| 2987.312 | 2987.5488 | 0.2368 | 79 | 389 | 414 | LCMGSGLNLCEPNNK |
| | | | | | | EG |
| | | | | | | YYGYTGAFR |

| | | | |
|---|---|---|---|
| Gel Idx/Pos | | 103/E2 | Instr./Gel Origin |
| Plate [#] Name | | [1] Sample Project | Instrument Sample |
| | | 20120802 | Name |
| Rank | Accession No. | Protein Name | |

| | |
|---|---|
| gi\|194380034 | unnamed protein product [Homo sapiens] |
| Protein Group | |
| gi\|6650772 | PRO1400 [Homo sapiens] |

Peptide Information

| | Obsrv. | | | Start | End | |
|---|---|---|---|---|---|---|
| Calc. Mass | Mass | ±da | ±ppm | Seq. | Seq. | Sequence |
| 887.4152 | 887.4493 | 0.0341 | 38 | 341 | 348 | SCHTAVGR |
| 964.5323 | 964.5878 | 0.0555 | 58 | 474 | 482 | APNHAVVTR |
| 1000.4985 | 1000.5601 | 0.0616 | 62 | 542 | 549 | YLGEEYVK |
| 1175.5803 | 1175.6743 | 0.094 | 80 | 533 | 541 | LHDRNTYEK |
| 1195.5426 | 1195.6356 | 0.093 | 78 | 236 | 244 | WCALSHHER |
| 1249.606 | 1249.7087 | 0.1027 | 82 | 327 | 337 | SASDLTWDNLK |
| 1283.5692 | 1283.6683 | 0.0991 | 77 | 404 | 414 | EGYYGYTGAFR |
| 1283.5692 | 1283.6683 | 0.0991 | 77 | 404 | 414 | EGYYGYTGAFR |
| 1323.7168 | 1323.8109 | 0.0941 | 71 | 189 | 200 | DSAHGFLKVPPR |
| 1377.7009 | 1377.7806 | 0.0797 | 58 | 326 | 337 | KSASDLTWDNLK |
| 1478.7349 | 1478.8579 | 0.123 | 83 | 205 | 216 | MYLGYEYVTAIR |
| 1478.7349 | 1478.8579 | 0.123 | 83 | 205 | 216 | MYLGYEYVTAIR |
| 1494.7297 | 1494.8467 | 0.117 | 78 | 205 | 216 | MYLGYEYVTAIR |
| 1494.7297 | 1494.8467 | 0.117 | 78 | 205 | 216 | MYLGYEYVTAIR |
| 1521.7367 | 1521.8425 | 0.1058 | 70 | 245 | 257 | LKCDEWSVNSVGK |
| 1531.688 | 1531.8191 | 0.1311 | 86 | 557 | 569 | CSTSSLLEACTFR |
| 1531.688 | 1531.8191 | 0.1311 | 86 | 557 | 569 | CSTSSLLEACTFR |
| 1539.7108 | 1539.8378 | 0.127 | 82 | 113 | 124 | DQYELLCLDNTR |
| 1565.7992 | 1565.9386 | 0.1394 | 89 | 520 | 532 | DLLFRDDTVCLAK |
| 1565.7992 | 1565.9386 | 0.1394 | 89 | 520 | 532 | DLLFRDDTVCLAK |
| 1577.8145 | 1577.8429 | 0.0284 | 18 | 349 | 362 | TAGWNIPMGLLYNK |

| | |
|---|---|
| Project\Project | 1577.842 |
| sample_20120802\P12139- | 9 |
| 2fncbi | |

| | | | | | | |
|---|---|---|---|---|---|---|
| 1586.7744 | 1586.9095 | 0.1351 | 85 | 461 | 473 | KPVEEYANCHLAR |
| 1586.7744 | 1586.9095 | 0.1351 | 85 | 461 | 473 | KPVEEYANCHLAR |
| 1659.783 | 1659.9297 | 0.1467 | 88 | 556 | 569 | KCSTSSLLEACTFR |
| 1689.849 | 1689.9705 | 0.1215 | 72 | 132 | 146 | DCHLAQVPSHTVVAR |
| 1705.7527 | 1705.9098 | 0.1571 | 92 | 368 | 382 | FDEFFSEGCAPGSKK |
| 1881.876 | 1882.0377 | 0.1617 | 86 | 110 | 124 | ADRDQYELLCLDNTR |
| 1952.9382 | 1953.1041 | 0.1659 | 85 | 445 | 460 | NLNEKDYELLCLDGTR |
| 1952.9382 | 1953.1041 | 0.1659 | 85 | 445 | 460 | NLNEKDYELLCLDGTR |
| 1974.9781 | 1975.1078 | 0.1297 | 66 | 427 | 444 | HQTVPQNTGGKNPDPW |
| | | | | | | AK |
| 2200.0227 | 2200.2285 | 0.2058 | 94 | 307 | 326 | SDNCEDTPEAGYFAVAV |
| | | | | | | VKK |
| 2201.0325 | 2201.1921 | 0.1596 | 73 | 217 | 235 | NLREGTCPEAPTDECKP |
| | | | | | | VK |
| 2258.1321 | 2258.1638 | 0.0317 | 14 | 349 | 367 | TAGWNIPMGLLYNKINHCR |
| 2549.293 | 2549.4946 | 0.2016 | 79 | 125 | 146 | KPVDEYKDCHLAQVPSH |
| | | | | | | TVVAR |
| 2549.293 | 2549.4946 | 0.2016 | 79 | 125 | 146 | KPVDEYKDCHLAQVPSH |
| | | | | | | TVVAR |
| 2971.3171 | 2971.5352 | 0.2181 | 73 | 389 | 414 | LCMGSGLNLCEPNNKEG |
| | | | | | | YYGYTGAFR |

| | | | |
|---|---|---|---|
| Gel Idx/Pos | | 108/E7 | Instr./Gel Origin |
| Plate [#] Name | | [1] Sample Project | Instrument Sample |
| | | 20120802 | Name |
| Rank | Accession No. | Protein Name | |

| | |
|---|---|
| gi\|194373497 | unnamed protein product [Homo sapiens] |
| | Peptide Information Project\ |
| | Project sample_20120802\ |
| | P12139-2fncbi |

| Calc. | Obsrv. | | | Start | End | |
|---|---|---|---|---|---|---|
| Mass | Mass | ±da | ±ppm | Seq. | Seq. | Sequence |
| 836.4009 | 836.3804 | −0.0205 | −25 | 67 | 73 | GQQYQGR |
| 836.4009 | 836.3804 | −0.0205 | −25 | 67 | 73 | GQQYQGR |
| 1189.579 | 1189.5811 | 0.0021 | 2 | 449 | 457 | YGFYTHVFR |
| 1189.579 | 1189.5811 | 0.0021 | 2 | 449 | 457 | YGFYTHVFR |
| 1194.6001 | 1194.5938 | −0.0063 | −5 | 203 | 212 | ELLESYIDGR |
| 1464.7805 | 1464.7512 | −0.0293 | −20 | 336 | 349 | ETAASLLQAGYKGR |
| 1489.7223 | 1489.7413 | 0.019 | 13 | 446 | 457 | DGKYGFYTHVFR |
| 1489.7223 | 1489.7413 | 0.019 | 13 | 446 | 457 | DGKYGFYTHVFR |
| 1515.8352 | 1515.8251 | −0.0101 | −7 | 290 | 301 | ISMLEKIYIHPR |
| 1518.8275 | 1518.6852 | −0.1423 | −94 | 265 | 277 | NFTENDLLVRIGK |
| 1526.6251 | 1526.655 | 0.0299 | 20 | 410 | 424 | GDACEGDSGGPFVMK |
| 1542.62 | 1542.7037 | 0.0837 | 54 | 410 | 424 | GDACEGDSGGPFVMK |
| 1561.7281 | 1561.7467 | 0.0186 | 12 | 164 | 176 | TATSEYQTFFNPR |
| 1561.7281 | 1561.7467 | 0.0186 | 12 | 164 | 176 | TATSEYQTFFNPR |
| 1645.8214 | 1645.8168 | −0.0046 | −3 | 234 | 248 | SPQELLCGASLISDR |
| 1682.7262 | 1682.7006 | −0.0256 | −15 | 409 | 424 | RGDACEGDSGGPFVMK |
| 1860.9603 | 1860.9722 | 0.0119 | 6 | 350 | 366 | VTGWGNLKETWTANVGK |
| 1883.8956 | 1883.9106 | 0.015 | 8 | 177 | 193 | TFGSGEADCGLRPLFEK |
| 1883.8956 | 1883.9106 | 0.015 | 8 | 177 | 193 | TFGSGEADCGLRPLFEK |
| 1963.908 | 1963.8922 | −0.0158 | −8 | 97 | 112 | HQDFNSAVQLVENFCR |
| 1995.0481 | 1995.0499 | 0.0018 | 1 | 74 | 92 | LAVTTHGLPCLAWASAQ |
| | | | | | | AK |
| 1995.0481 | 1995.0499 | 0.0018 | 1 | 74 | 92 | LAVTTHGLPCLAWASAQ |
| | | | | | | AK |
| 2001.9158 | 2001.9205 | 0.0047 | 2 | 393 | 409 | ITDNMFCAGYKPDEGKR |
| 2011.9906 | 2012.0179 | 0.0273 | 14 | 177 | 194 | TFGSGEADCGLRPLFEKK |
| 2011.9906 | 2012.0179 | 0.0273 | 14 | 177 | 194 | TFGSGEADCGLRPLFEKK |
| 2017.9106 | 2018.0586 | 0.148 | 73 | 393 | 409 | ITDNMFCAGYKPDEGKR |
| 2232.2534 | 2232.2668 | 0.0134 | 6 | 367 | 386 | GQPSVLQVVNLPIVERPV |
| | | | | | | CK |
| 2232.2534 | 2232.2668 | 0.0134 | 6 | 367 | 386 | GQPSVLQVVNLPIVERPV |
| | | | | | | CK |
| 2281.0991 | 2281.1819 | 0.0828 | 36 | 213 | 232 | IVEGSDAEIGMSPWQVM |
| | | | | | | LFR |
| 2888.3591 | 2888.3918 | 0.0327 | 11 | 48 | 73 | SEGSSVNLSPPLEQCVP |
| | | | | | | DRGQQYQGR |
| 2888.3591 | 2888.3918 | 0.0327 | 11 | 48 | 73 | SEGSSVNLSPPLEQCVP |
| | | | | | | DRGQQYQGR |

### Figure Descriptions

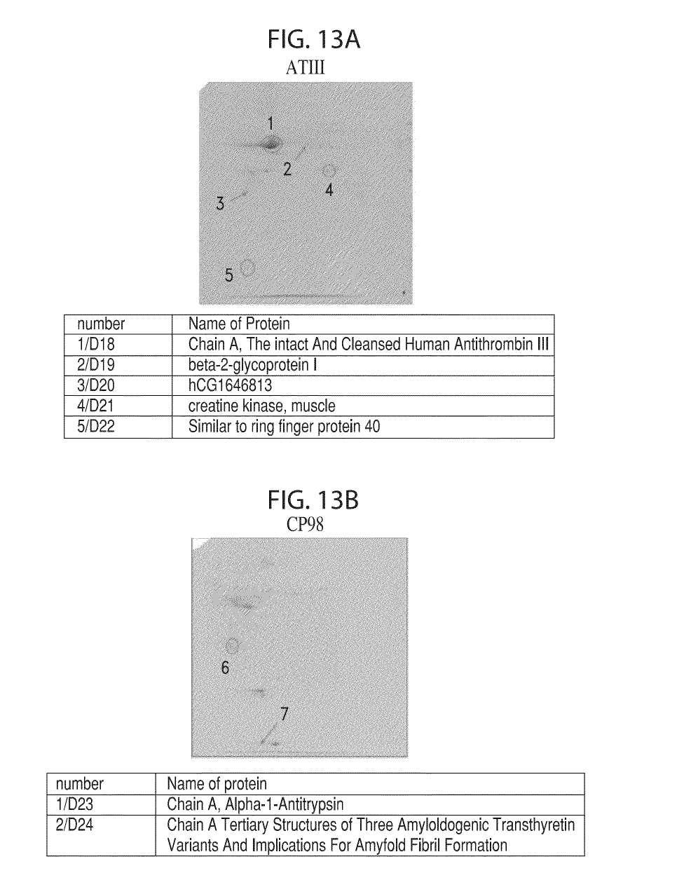

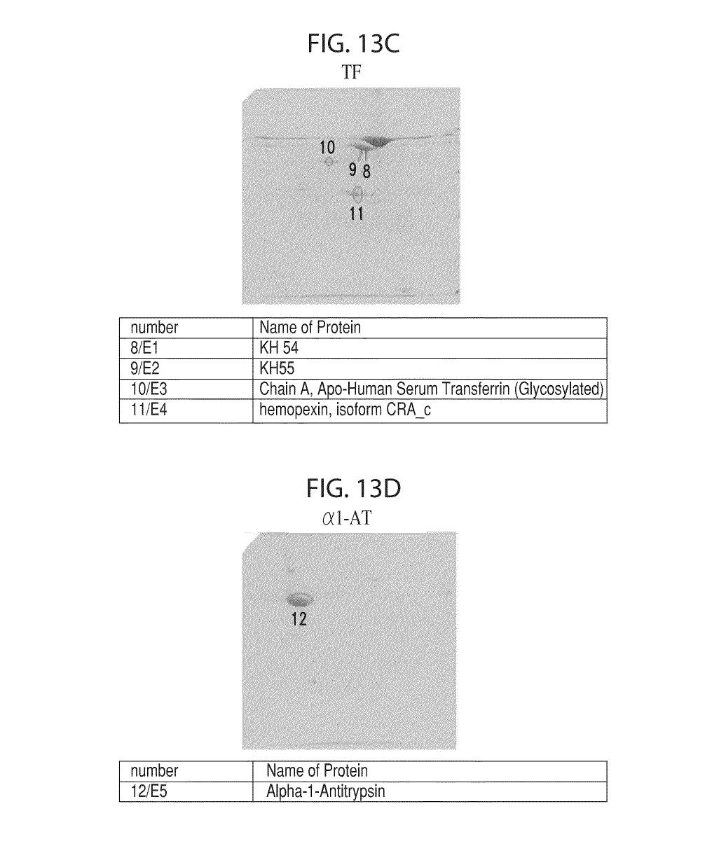

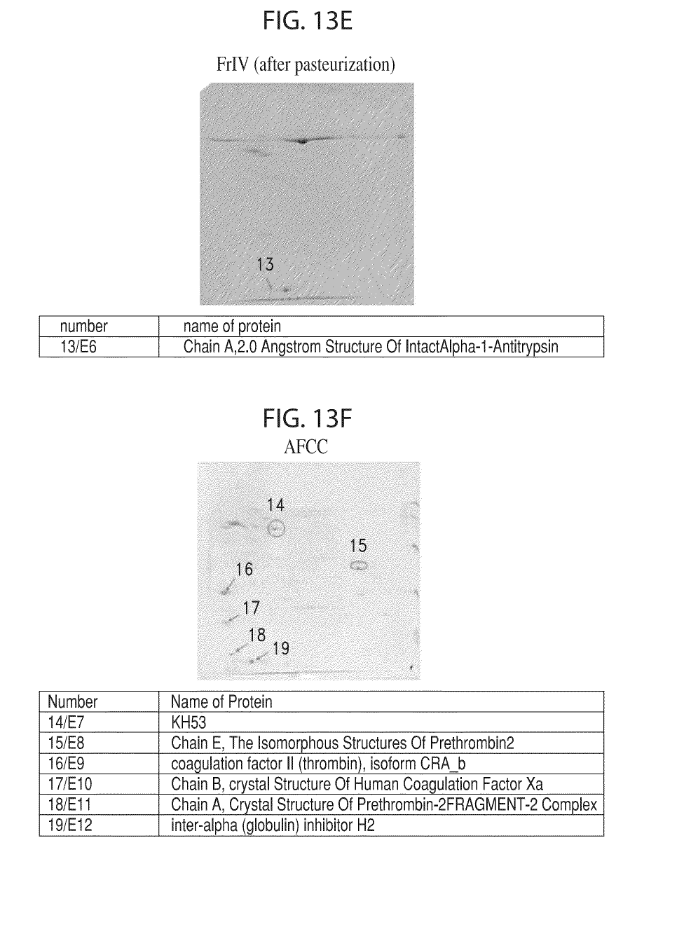

FIG. 13A—2D Electrophoresis of Anti Thrombin III

FIG. 13B—2D Electrophoresis of CP98

FIG. 13C—2D Electrophoresis of Transferrin

FIG. 13D—2D Electrophoresis of α1-Anti Trypsin

FIG. 13E—2D Electrophoresis of Human Albumin after pasteurization from Fraction IV

FIG. 13F—2D Electrophoresis of AFCC®

Along with these three newly discovered proteins for all the products derived from fraction IV, we have found the following existing discovered proteins from Anti Thrombin III in which in which good KH healthy cells exist from Anti Thrombin III protein and their sequence:
Chain A, The Intact And Cleaved Human Antithrombin III
Beta-2-glycoprotein I
hCG1646813
Creatine kinase, muscle
Similar to ring finger protein 40

### FIG. 13A

| | | | |
|---|---|---|---|
| Gel Idx/Pos | | 93/D18 | Instr./Gel Origin |
| Plate [#] Name | | [1] Sample Project | Instrument Sample |
| | | 20120802 | Name |
| Rank | Accession No. | Protein Name | |

| | |
|---|---|
| gi\|28580349 | Chain I, Crystal Structure Of |
| | The Antithrombin-Factor 4 |
| | Ixa-Pentasaccharide Complex |
| Protein Group | |
| gi\|999513 | Chain A, The Intact And |
| | Cleaved Human Antithrombin |
| | Iii Complex As A Model |
| | For Serpin-Proteinase Interactions |

Peptide Information

| | Obsrv. | | | Start | End | |
|---|---|---|---|---|---|---|
| Calc. Mass | Mass | ±da | ±ppm | Seq. | Seq. | Sequence |
| 917.5203 | 917.4625 | −0.0578 | −63 | 47 | 53 | RVWELSK |
| 1065.5323 | 1065.4781 | −0.0542 | −51 | 227 | 235 | SKFSPENTR |
| 1175.6532 | 1175.6271 | −0.0261 | −22 | 394 | 403 | SLNPNRVTFK |
| 1211.642 | 1211.6008 | −0.0412 | −34 | 323 | 332 | FRIEDGFSLK |
| 1211.642 | 1211.6008 | −0.0412 | −34 | 323 | 332 | FRIEDGFSLK |
| 1232.7263 | 1232.6871 | −0.0392 | −32 | 404 | 413 | ANRPFLVFIR |
| 1232.7263 | 1232.6871 | −0.0392 | −32 | 404 | 413 | ANRPFLVFIR |
| 1340.6635 | 1340.6451 | −0.0184 | −14 | 115 | 125 | TSDQIHFFFAK |
| 1340.6635 | 1340.6451 | −0.0184 | −14 | 115 | 125 | TSDQIHFFFAK |
| 1389.7559 | 1389.731 | −0.0249 | −18 | 414 | 425 | EVPLNTIIFMGR |
| 1389.7559 | 1389.731 | −0.0249 | −18 | 414 | 425 | EVPLNTIIFMGR |

Project\Project sample_{—}20120802\P12139-2fncbi

| | | | | | | |
|---|---|---|---|---|---|---|
| 1405.7509 | 1405.7101 | −0.0408 | −29 | 414 | 425 | EVPLNTIIFMGR |
| 1409.6375 | 1409.589 | −0.0485 | −34 | 14 | 24 | DIPMNPMCIYR |
| 1409.6375 | 1409.589 | −0.0485 | −34 | 14 | 24 | DIPMNPMCIYR |
| 1425.6323 | 1425.6094 | −0.0229 | −16 | 14 | 24 | DIPMNPMCIYR |
| 1425.6323 | 1425.6094 | −0.0229 | −16 | 14 | 24 | DIPMNPMCIYR |
| 1437.7268 | 1437.6921 | −0.0347 | −24 | 1 | 13 | HGSPVDICTAKPR |
| 1437.7268 | 1437.6921 | −0.0347 | −24 | 1 | 13 | HGSPVDICTAKPR |
| 1441.6273 | 1441.6387 | 0.0114 | 8 | 14 | 24 | DIPMNPMCIYR |
| 1528.7432 | 1528.736 | −0.0072 | −5 | 58 | 70 | FATTFYQHLADSK |
| 1674.8446 | 1674.7909 | −0.0537 | −32 | 170 | 183 | LQPLDFKENA |
| | | | | | | EQSR |
| 1674.8446 | 1674.7909 | −0.0537 | −32 | 170 | 183 | LQPLDFKENAE |
| | | | | | | QSR |
| 1708.0057 | 1707.9534 | −0.0523 | −31 | 400 | 413 | VTFKANRPFL |
| | | | | | | VFIR |
| 2202.1189 | 2202.054 | −0.0649 | −29 | 351 | 370 | LPGIVAEGRD |
| | | | | | | DLYVSDAFHK |
| 2291.1514 | 2291.0154 | −0.136 | −59 | 371 | 393 | AFLEVNEEGSE |
| | | | | | | AAASTAV |

| | | | |
|---|---|---|---|
| Gel Idx/Pos | | 94/D19 | Instr./Gel Origin |
| Plate [#] Name | | [1] Sample Project | Instrument Sample |
| | | 20120802 | Name |
| Rank | Accession No. | Protein Name | |

| | |
|---|---|
| gi\|300253216 | beta-2-glycoprotein I [Homo sapiens] |
| Protein Group | |
| gi\|6573461 | Chain A, Crystal Structure Of |
| | Human Beta-2-Glycoprotein-I |
| | (Apolipoprotein-H) |

Peptide Information

| | Obsrv. | | | Start | End | |
|---|---|---|---|---|---|---|
| Calc. Mass | Mass | ±da | ±ppm | Seq. | Seq. | Sequence |
| 1022.5266 | 1022.4513 | −0.0753 | −74 | 252 | 260 | ATVVYQGER |
| 1022.5266 | 1022.4513 | −0.0753 | −74 | 252 | 260 | ATVVYQGER |
| 1104.5472 | 1104.4421 | −0.1051 | −95 | 309 | 317 | EHSSLAFWK |
| 1150.6216 | 1150.5341 | −0.0875 | −76 | 251 | 260 | KATVVYQGER |
| 1249.6899 | 1249.6323 | −0.0576 | −46 | 252 | 262 | ATVVYQGERVK |
| 1502.7784 | 1502.6923 | −0.0861 | −57 | 64 | 77 | VCPFAGILENGAVR |

Project\Project sample_{—}20120802\P12139-2fncbi

| | | | | | | |
|---|---|---|---|---|---|---|
| 1502.7784 | 1502.6923 | −0.0861 | −57 | 64 | 77 | VCPFAGILEN |
| | | | | | | GAVR |
| 1539.7413 | 1539.6655 | −0.0758 | −49 | 306 | 317 | CFKEHSSLAFWK |
| 2127.1267 | 2126.9858 | −0.1409 | −66 | 1 | 19 | GRTCPKPDDLPF |
| | | | | | | STVVPLK |
| 2383.0911 | 2382.9473 | −0.1438 | −60 | 20 | 39 | TFYEPGEEIT |
| | | | | | | YSCKPGYVSR |
| 2731.3337 | 2731.199 | −0.1347 | −49 | 186 | 208 | CPFPSRPDNG |
| | | | | | | FVNYPAKPTLYYK |
| 2974.4556 | 2974.2261 | −0.2295 | −77 | 186 | 210 | CPFPSRPD |
| | | | | | | NGFVNYPAK |

| Gel Idx/Pos | | 95/D20 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|119622576 | hCG1646813 | |
| | | [Homo sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 842.3978 | 842.4335 | 0.0357 | 42 | 33 | 39 | GPMHWSK |
| 1428.8461 | 1428.8052 | −0.0409 | −29 | 103 | 116 | LIGVFLDSGLVPAK |
| 1956.8352 | 1956.936 | 0.1008 | 52 | 2 | 18 | GCAQQQTTEEYRDGADK |
| 1960.1073 | 1960.0238 | −0.0835 | −43 | 62 | 81 | SGTTKLEALAGIALLATSSR |
| 1960.1073 | 1960.0238 | −0.0835 | −43 | 62 | 81 | SGTTKLEALAGIALLATSSR |

| Gel Idx/Pos | | 96/D21 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|119577741 | creatine kinase, | |
| | | muscle [Homo | |
| | | sapiens] | |

Peptide Information

| | Obsrv. | | | Start | End | |
|---|---|---|---|---|---|---|
| Calc. Mass | Mass | ±da | ±ppm | Seq. | Seq. | Sequence |
| 851.394 | 851.379 | −0.015 | −18 | 51 | 57 | HNNHMAK |
| | 851.379 | | | | | |

Project\Project sample_{—}20120802\P12139-2fncbi

| | | | | | | |
|---|---|---|---|---|---|---|
| 868.4927 | 868.5339 | 0.0412 | 47 | 196 | 202 | GKYYPLK |
| 907.4883 | 907.4175 | −0.0708 | −78 | 333 | 339 | FEEILTR |
| 907.4883 | 907.4175 | −0.0708 | −78 | 333 | 339 | FEEILTR |
| 1507.7024 | 1507.5952 | −0.1072 | −71 | 142 | 155 | GGDDLD |
| | | | | | | PNYVLSSR |
| 1643.8176 | 1643.7117 | −0.1059 | −64 | 249 | 261 | SFLVWVNE |
| | | | | | | EDHLR |
| 1994.9415 | 1994.7739 | −0.1676 | −84 | 346 | 366 | GTGGVDTA |
| | | | | | | AVGSVFDVSNA |
| | | | | | | DR |
| 1994.9415 | 1994.7739 | −0.1676 | −84 | 346 | 366 | GTGGVDTAA |
| | | | | | | VGSVFDVS |

| Gel Idx/Pos | | 97/D22 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|28175093 | Similar to ring | |
| | | finger protein 40 | |
| | | [Homo sapiens] | |

Peptide Information

| Calc. | Obsrv. | | | Start | End | |
|---|---|---|---|---|---|---|
| Mass | Mass | ±da | ±ppm | Seq. | Seq. | Sequence |
| 1257.6686 | 1257.697 | 0.0284 | 23 | 523 | 534 | AVEAAQLAEDLK |
| 1385.7634 | 1385.673 | −0.0904 | −65 | 522 | 534 | KAVEAAQLAEDLK |
| 1649.8718 | 1649.7906 | −0.0812 | −49 | 390 | 402 | IRQAEEQIEHLQR |
| 1663.8286 | 1663.7806 | −0.048 | −29 | 36 | 49 | SLPEEVVRETGEYR |
| 1681.785 | 1681.7722 | −0.0128 | −8 | 86 | 99 | HIEHMESDELGLQK |
| 1748.9099 | 1748.7834 | −0.1265 | −72 | 223 | 239 | EMAPVPGTTTTTTSVKK |
| 1748.9099 | 1748.7834 | −0.1265 | −72 | 222 | 238 | KEMAPVPGTTTTTTSVK |
| 1773.7771 | 1773.8884 | 0.1113 | 63 | 623 | 635 | CFHVFCFECVRGR |
| 1801.9476 | 1801.9485 | 0.0009 | 0 | 10 | 25 | MAELEKLQAELQGAVR |

Project\Project sample_{—}20120802\P12139-2fncbi

| | | | | | | |
|---|---|---|---|---|---|---|
| 1819.9661 | 1819.937 | −0.0291 | −16 | 276 | 293 | EREGPSLGPPPVA |
| | | | | | | SALSR |
| 1819.9661 | 1819.937 | −0.0291 | −16 | 276 | 293 | EREGPSLGPPPV |
| | | | | | | ASALSR |
| 2348.2568 | 2348.1741 | −0.0827 | −35 | 523 | 543 | AVEAAQLAEDLK |
| | | | | | | VQLEHV |

Along with these three newly discovered proteins for all the products derived from fraction IV, we have found the following existing discovered proteins from CP98 in which in which good KH healthy cells exist from CP98 protein and their sequence:
Chain A, Alpha-1-Antitrypsin
Chain A, Tertiary Structures Of Three Amyloidogenic Transthyretin Variants And Implications For Amyloid Fibril Formation

### FIG. 13B

Protein Group

| Gel Idx/Pos | | 98/D23 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|196049621 | Chain A, Crystal | |
| | | Structure Of Alpha- | |
| | | 1-Antitrypsin, | |
| | | Crystal Form A | |
| | gi\|157831596 | Chain A, Alpha1- | |
| | | Antitrypsin | |

Peptide Information

| | Obsrv. | | | Start | End | |
|---|---|---|---|---|---|---|
| Calc. Mass | Mass | ±da | ±ppm | Seq. | Seq. | Sequence |
| 1008.4996 | 1008.4 | −0.0996 | −99 | 156 | 163 | QINDYVEK |
| 1058.475 | 1058.3909 | −0.0841 | −79 | 1 | 10 | EDPQGDAAQK |
| 1058.475 | 1058.3909 | −0.0841 | −79 | 1 | 10 | EDPQGDAAQK |
| 1136.5946 | 1136.5233 | −0.0713 | −63 | 155 | 163 | KQINDYVEK |
| 1275.6844 | 1275.583 | −0.1014 | −79 | 192 | 201 | GKWERPFEVK |
| 1479.7438 | 1479.6862 | −0.0576 | −39 | 156 | 168 | QINDYVEKGTQGK |
| 1641.8635 | 1641.8329 | −0.0306 | −19 | 26 | 39 | ITPNLAEFAFSLYR |
| 1641.8635 | 1641.8329 | −0.0306 | −19 | 26 | 39 | ITPNLAEFAFSLYR |
| 1779.7682 | 1779.7164 | −0.0518 | −29 | 11 | 25 | TDTSHHDQDHPTFNK |
| 1779.7682 | 1779.7164 | −0.0518 | −29 | 11 | 25 | TDTSHHDQDHPTFNK |
| 1891.8557 | 1891.8124 | −0.0433 | −23 | 202 | 217 | DTEEEDFHVDQVTTVK |
| 1891.8557 | 1891.8124 | −0.0433 | −23 | 202 | 217 | DTEEEDFHVDQVTTVK |
| 2186.04 | 2186.03 | −0.01 | −5 | 137 | 155 | LYHSEAFTVNFGDTEEAKK |
| 2186.04 | 2186.03 | −0.01 | −5 | 137 | 155 | LYHSEAFTVNFGDTEEAKK |
| 2574.3411 | 2574.3967 | 0.0556 | 22 | 102 | 125 | TLNQPDSQLQLTTGNGL |
| | | | | | | FLSEGLK |
| 2574.3411 | 2574.3967 | 0.0556 | 22 | 102 | 125 | TLNQPDSQLQLTTGNGL |
| | | | | | | FLSEGLK |

| Gel Idx/Pos | | 99/D24 | Instr./Gel Origin |
|---|---|---|---|
| Plate [#] | | [1] Sample Project | Instrument |
| Name | | 20120802 | Sample Name |
| Rank | Accession No. | Protein Name | |
| | gi\|3891560 | Chain A, Tertiary | |
| | | Structures Of Three | |
| | | Amyloidogenic | |
| | | Transthyretin Variants | |
| | | And Implications | |
| | | For Amyloid | |
| | | Fibril Formation | |

Peptide Information

| Calc. | Obsrv. | | | Start | End | |
|---|---|---|---|---|---|---|
| Mass | Mass | ±da | ±ppm | Seq. | Seq. | Sequence |
| 863.4105 | 863.3267 | −0.0838 | −97 | 1 | 9 | GPTGTSESK |
| 1394.6223 | 1394.5464 | −0.0759 | −54 | 36 | 48 | AADDTWEPFASGK |
| 1394.6223 | 1394.5464 | −0.0759 | −54 | 36 | 48 | AADDTWEPFASGK |
| 1494.854 | 1494.7693 | −0.0847 | −57 | 22 | 35 | GSPAINVAVHVFRK |
| 1522.7173 | 1522.6631 | −0.0542 | −36 | 35 | 48 | KAADDTWEPFASGK |
| 1522.7173 | 1522.6631 | −0.0542 | −36 | 35 | 48 | KAADDTWEPFASGK |
| 2451.2053 | 2451.0967 | −0.1086 | −44 | 81 | 103 | ALGISPFHEHAEVVFTAN |
| | | | | | | DSGPR |

Project\Project sample_{—}20120802\P12139-2fncbi

| | | | | | | |
|---|---|---|---|---|---|---|
| 2451.2053 | 2451.0967 | −0.1086 | −44 | 81 | 103 | ALGISPFHEHAEV |
| | | | | | | VFTANDSGPR |
| 2455.1511 | 2455.0598 | −0.0913 | −37 | 49 | 70 | TSESGELHGLTT |
| | | | | | | EEEFVEGIYK |
| 2607.3064 | 2607.1531 | −0.1533 | −59 | 81 | 104 | ALGISPFHEHAEV |
| | | | | | | VFTANDSGPRR |
| 3140.5159 | 3140.3345 | −0.1814 | −58 | 49 | 76 | TSESGELHGLTTE |
| | | | | | | EEFVEGIYKVEI |
| | | | | | | DTK |
| 3140.5159 | 3140.3345 | −0.1814 | −58 | 49 | 76 | TSESGELHGLTTE |
| | | | | | | EEFVEGIYKVEI |
| | | | | | | DTK |

Along with these three newly discovered proteins for all the products derived from fraction IV, we have found the following existing discovered proteins from Transferrin in which in which good KH healthy cells exist from Transferrin protein and their sequence:
KH54
KH55
Chain A, Apo-Human Serum Transferrin (Glycosylated)
hemopexin, isoform CRA_c

### FIG. 13C

| Gel Idx/Pos | | 104/E3 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|110590599 | Chain A, Apo- | |
| | | Human Serum | |
| | | Transferrin | |
| | | (Glycosylated) | |

Peptide information

| Calc. | Obsrv. | | | Start | End | |
|---|---|---|---|---|---|---|
| Mass | Mass | ±da | ±ppm | Seq. | Seq. | Sequence |
| 874.4417 | 874.445 | 0.0033 | 4 | 294 | 301 | DSAHGFLK |
| 887.4152 | 887.4274 | 0.0122 | 14 | 446 | 453 | SCHTAVGR |
| 997.4771 | 997.5176 | 0.0405 | 41 | 40 | 47 | ASYLDCIR |
| 997.4771 | 997.5176 | 0.0405 | 41 | 40 | 47 | ASYLDCIR |
| 1125.5721 | 1125.6246 | 0.0525 | 47 | 39 | 47 | KASYLD |
| | | | | | | CIR |

Project\Project sample_{—}20120802T12139-2fncbi

| | | | | | | |
|---|---|---|---|---|---|---|
| 1125.6198 | 1125.6246 | 0.0048 | 4 | 591 | 599 | EACVHKILR |
| 1195.5525 | 1195.6206 | 0.0681 | 57 | 101 | 110 | DSGFQMNQLR |
| 1195.5525 | 1195.6206 | 0.0681 | 57 | 101 | 110 | DSGFQMNQLR |
| 1208.5299 | 1208.5984 | 0.0685 | 57 | 16 | 24 | CQSFRDHMK |
| 1211.5474 | 1211.606 | 0.0586 | 48 | 101 | 110 | DSGFQMNQLR |
| 1211.5474 | 1211.606 | 0.0586 | 48 | 101 | 110 | DSGFQMNQLR |
| 1224.5249 | 1224.5942 | 0.0693 | 57 | 16 | 24 | CQSFRDHMK |
| 1249.606 | 1249.6862 | 0.0802 | 64 | 432 | 442 | SASDLTWDNLK |
| 1317.5892 | 1317.6625 | 0.0733 | 56 | 5 | 15 | WCAVSEHEATK |
| 1323.7168 | 1323.748 | 0.0312 | 24 | 294 | 305 | DSAHGFLKVPPR |
| 1323.7168 | 1323.748 | 0.0312 | 24 | 294 | 305 | DSAHGFLKVPPR |
| 1339.6423 | 1339.714 | 0.0717 | 54 | 100 | 110 | KDSGFQMNQLR |
| 1379.6989 | 1379.7874 | 0.0885 | 64 | 191 | 203 | CLKDGAGDVAFVK |
| 1415.72 | 1415.8167 | 0.0967 | 68 | 25 | 38 | SVIPSDGPSVACVK |
| 1478.7349 | 1478.8274 | 0.0925 | 63 | 310 | 321 | MYLGYEYVTAIR |
| 1478.7349 | 1478.8274 | 0.0925 | 63 | 310 | 321 | MYLGYEYVTAIR |
| 1494.7297 | 1494.8149 | 0.0852 | 57 | 310 | 321 | MYLGYEYVTAIR |
| 1494.7297 | 1494.8149 | 0.0852 | 57 | 310 | 321 | MYLGYEYVTAIR |
| 1521.7367 | 1521.8673 | 0.1306 | 86 | 350 | 362 | LKCDEWSVNSVGK |
| 1817.8044 | 1817.906 | 0.1016 | 56 | 325 | 340 | EGTCPEAPTDECK |
| | | | | | | PVK |
| 1881.876 | 1881.9436 | 0.0676 | 36 | 215 | 229 | ADRDQYELLCL |
| | | | | | | DNTR |
| 2071.9277 | 2072.0383 | 0.1106 | 53 | 412 | 430 | SDNCEDTPEAGYF |
| | | | | | | AVAVVK |
| 2159.0149 | 2159.1138 | 0.0989 | 46 | 378 | 398 | IMNGEADAMSLDG |
| | | | | | | GFVYIAGK |
| 2171.0955 | 2171.231 | 0.1355 | 62 | 122 | 140 | SAGWNIPIGLLYC |
| | | | | | | DLPEPR |
| 2175.0098 | 2175.1689 | 0.1591 | 73 | 378 | 398 | IMNGEADAMSLDG |
| | | | | | | GFVYIAGK |
| 2191.0046 | 2191.0989 | 0.0943 | 43 | 378 | 398 | IMNGEADAMSLDG |
| | | | | | | GFVYIAGK |
| 2201.0325 | 2201.1443 | 0.1118 | 51 | 322 | 340 | NLREGTCPEAPTD |
| | | | | | | ECKPVK |
| 2549.293 | 2549.4204 | 0.1274 | 50 | 230 | 251 | KPVDEYKDCHLAQ |
| | | | | | | VPSHTVVAR |
| 2549.293 | 2549.4204 | 0.1274 | 50 | 230 | 251 | KPVDEYKDCHLAQ |
| | | | | | | VPSHTVVAR |

| Gel Idx/Pos | | 105/E4 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|119589126 | hemopexin, isoform | |
| | | CRA_c | |
| | | [Homo sapiens] | |

Peptide Information

| | | | | Start | End | |
|---|---|---|---|---|---|---|
| Calc. Mass | Obsrv. Mass | ±da | ±ppm | Seq. | Seq. | Sequence |
| 813.4399 | 813.4396 | −0.0003 | 0 | 170 | 176 | LHIMAGR |
| 813.4399 | 813.4396 | −0.0003 | 0 | 170 | 176 | LHIMAGR |
| 953.5461 | 953.564 | 0.0179 | 19 | 170 | 177 | LHIMAGRR |
| 969.5411 | 969.6055 | 0.0644 | 66 | 170 | 177 | LHIMAGRR |
| 1022.4645 | 1022.4827 | 0.0182 | 18 | 201 | 209 | VDGALCMEK |
| 1038.4595 | 1038.5132 | 0.0537 | 52 | 201 | 209 | VDGALCMEK |
| 1129.6517 | 1129.6848 | 0.0331 | 29 | 177 | 184 | RLWWLDLK |
| 1141.5889 | 1141.6267 | 0.0378 | 33 | 131 | 141 | GGYTLVSGYPK |
| 1141.5889 | 1141.6267 | 0.0378 | 33 | 131 | 141 | GGYTLVSGYPK |
| 1837.8868 | 1837.9629 | 0.0761 | 41 | 185 | 200 | SGAQATWTELPWPHEK |
| 1837.8868 | 1837.9629 | 0.0761 | 41 | 185 | 200 | SGAQATWTELPWPHEK |
| 1851.7939 | 1851.9684 | 0.1745 | 94 | 38 | 54 | NGTGHGNSTHHGPEYMR |

Project\Project sample_{—}20120802\P12139-2fncbi

| | | | | | | |
|---|---|---|---|---|---|---|
| 2498.2344 | 2498.3313 | 0.0969 | 39 | 146 | 169 | EVGTPHGIILDSVDAAFIC |
| | | | | | | PGSSR |
| 2498.2344 | 2498.3313 | 0.0969 | 39 | 146 | 169 | EVGTPHGIILDSVDAAFIC |
| | | | | | | PGSSR |
| 2821.2349 | 2821.5139 | 0.279 | 99 | 1 | 24 | MGPGVNLREMEEFPQPC |
| | | | | | | GCCIPSR |
| 2841.3335 | 2841.427 | 0.0935 | 33 | 185 | 209 | SGAQATWTELPWPHEKV |
| | | | | | | DGALCMEK |
| 2853.2246 | 2853.4355 | 0.2109 | 74 | 1 | 24 | MGPGVNLREMEEFPQPC |
| | | | | | | GCCIPSR |
| 2857.3284 | 2857.0476 | −0.2808 | −98 | 185 | 209 | SGAQATWTELPWPHEKV |
| | | | | | | DGALCMEK |
| 2857.3284 | 2857.4575 | 0.1291 | 45 | 185 | 209 | SGAQATWTELPWPHEKV |
| | | | | | | DGALCMEK |
| 3282.5195 | 3282.6726 | 0.1531 | 47 | 210 | 239 | SLGPNSCSANGPGLYLIH |
| | | | | | | GPNLYCYSDVEK |
| 3779.8157 | 3779.9861 | 0.1704 | 45 | 210 | 244 | SLGPNSCSANGPGLYLIH |
| | | | | | | GPNLYCYSDVEKLNAAK |
| 3779.8157 | 3779.9861 | 0.1704 | 45 | 210 | 244 | SLGPNSCSANGPGLYLIH |
| | | | | | | GPNLYCYSDVEKLNAAK |

Along with these three newly discovered proteins for all the products derived from fraction IV, we have found the following existing discovered proteins from α1-Anti Trypsin in which in which good KH healthy cells exist from α1-Anti Trypsin protein and their sequence:
Alpha-1-Antitrypsin

### FIG. 13D

| Gel Idx/Pos | | 106/E5 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|6137432 | Chain A, 2.0 | |
| | | Angstrom Structure | |
| | | Of Intact | |
| | | Alpha-1- | |
| | | Antitrypsin: A | |
| | | Canonical Template | |
| | | For Active Serpins | |

Peptide Information

| Calc. | Obsrv. | | | Start | End | |
|---|---|---|---|---|---|---|
| Mass | Mass | ±da | ±ppm | Seq. | Seq. | Sequence |
| 1078.5276 | 1078.5848 | 0.0572 | 53 | 275 | 282 | FLENEDRR |
| 1078.5276 | 1078.5848 | 0.0572 | 53 | 275 | 282 | FLENEDRR |
| 1204.7123 | 1204.7484 | 0.0361 | 30 | 234 | 243 | KLSSWVLL |
| | | | | | | MK |
| 1247.6024 | 1247.6445 | 0.0421 | 34 | 224 | 233 | LGMFNIQH |
| | | | | | | CK |
| 1275.6844 | 1275.7573 | 0.0729 | 57 | 192 | 201 | GKWERPFE |
| | | | | | | VK |
| 1275.6844 | 1275.7573 | 0.0729 | 57 | 192 | 201 | GKWERPFE |
| | | | | | | VK |
| 1403.7035 | 1403.7723 | 0.0688 | 49 | 223 | 233 | RLGMFNIQ |
| | | | | | | HCK |

Project\Project sample_{—}20120802\P12139-2fncbi

| | | | | | | |
|---|---|---|---|---|---|---|
| 1641.8635 | 1641.9781 | 0.1146 | 70 | 26 | 39 | ITPNLAEFAFSLYR |
| 1641.8635 | 1641.9781 | 0.1146 | 70 | 26 | 39 | ITPNLAEFAFSLYR |
| 1779.7682 | 1779.8448 | 0.0766 | 43 | 11 | 25 | TDTSHHDQDHPTFNK |
| 1855.9775 | 1856.0656 | 0.0881 | 47 | 366 | 380 | FNKPFVFLMIEQNTK |
| 1871.9724 | 1872.0657 | 0.0933 | 50 | 366 | 380 | FNKPFVFLMIEQNTK |
| 2057.9451 | 2057.9675 | 0.0224 | 11 | 137 | 154 | LYHSEAFTVNFGDTEEAK |
| 2090.0957 | 2090.1877 | 0.092 | 44 | 175 | 191 | ELDRDTVFALVNYIFFK |
| 2186.04 | 2186.1306 | 0.0906 | 41 | 137 | 155 | LYHSEAFTVNFGDTEEAKK |
| 2186.04 | 2186.1306 | 0.0906 | 41 | 137 | 155 | LYHSEAFTVNFGDTEEAKK |
| 2259.1399 | 2259.2156 | 0.0757 | 34 | 344 | 365 | GTEAAGAMFLEAIPMSIP |
| | | | | | | PEVK |
| 2259.1399 | 2259.2156 | 0.0757 | 34 | 344 | 365 | GTEAAGAMFLEAIPMSIP |
| | | | | | | PEVK |
| 2574.3411 | 2574.5083 | 0.1672 | 65 | 102 | 125 | TLNQPDSQLQLTTGNGL |
| | | | | | | FLSEGLK |
| 2963.4058 | 2963.5151 | 0.1093 | 37 | 194 | 217 | WERPFEVKDTEEEDFHV |
| | | | | | | DQVTTVK |
| 3128.6257 | 3128.7175 | 0.0918 | 29 | 336 | 365 | AVLTIDEKGTEAAGAMFL |
| | | | | | | EAIPMSIPPEVK |
| 3160.6157 | 3160.6826 | 0.0669 | 21 | 336 | 365 | AVLTIDEKGTEAAGAMFL |
| | | | | | | EAIPMSIPPEVK |
| 3181.635 | 3181.6121 | −0.0229 | −7 | 40 | 69 | QLAHQSNSTNIFFSPVSIA |
| | | | | | | TAFAMLSLGTK |

Along with these three newly discovered proteins for all the products derived from fraction IV, we have found the following existing discovered proteins from Human Albumin after pasteurization in which in which good KH healthy cells exist Human Albumin after pasteurization protein and their sequence:
Chain A, 2.0 Angstrom Structure Of IntactAlpha-1-Antitrypsin

### FIG. 13C

| Gel Idx/Pos | | 107/E6 | Instr./Gel Origin |
|---|---|---|---|
| Plate [#] | | [1] Sample Project | Instrument |
| Name | | 20120802 | Sample Name |
| Rank | Accession No. | Protein Name | |
| | gi\|6137432 | Chain A, 2.0 | |
| | | Angstrom Structure | |
| | | Of Intact | |
| | | Alpha-1-Antitrypsin: | |
| | | A Canonical | |
| | | Template For Active | |
| | | Serpins | |
| | Protein Group | | |
| | gi\|157831596 | Chain A, Alpha1- | |
| | | Antitrypsin | |

Peptide Information

| | Obsrv. | | | Start | End | |
|---|---|---|---|---|---|---|
| Calc. Mass | Mass | ±da | ±ppm | Seq. | Seq. | Sequence |
| 1076.6173 | 1076.589 | −0.0283 | −26 | 235 | 243 | LSSWVLLMK |
| 1078.5276 | 1078.5682 | 0.0406 | 38 | 275 | 282 | FLENEDRR |
| 1078.5276 | 1078.5682 | 0.0406 | 38 | 275 | 282 | FLENEDRR |
| 1247.6024 | 1247.6372 | 0.0348 | 28 | 224 | 233 | LGMFNIQHCK |
| 1263.5973 | 1263.65 | 0.0527 | 42 | 224 | 233 | LGMFNIQHCK |
| 1323.7631 | 1323.6909 | −0.0722 | −55 | 332 | 343 | AVHKAVLTIDEK |
| 1641.8635 | 1641.8929 | 0.0294 | 18 | 26 | 39 | ITPNLAEFAFSLYR |
| 1641.8635 | 1641.8929 | 0.0294 | 18 | 26 | 39 | ITPNLAEFAFSLYR |
| 1779.7682 | 1779.7826 | 0.0144 | 8 | 11 | 25 | TDTSHHDQDHPTFNK |
| 1779.7682 | 1779.7826 | 0.0144 | 8 | 11 | 25 | TDTSHHDQDHPTFNK |
| 1803.96 | 1804.0195 | 0.0595 | 33 | 260 | 274 | LQHLENELTHDIITK |
| 1833.923 | 1833.8859 | −0.0371 | −20 | 311 | 328 | VFSNGADLSGVTEEAPLK |
| 1855.9775 | 1855.9918 | 0.0143 | 8 | 366 | 380 | FNKPFVFLMIEQNTK |
| 1891.8557 | 1891.8741 | 0.0184 | 10 | 202 | 217 | DTEEEDFHVDQVTTVK |
| 2186.04 | 2186.0742 | 0.0342 | 16 | 137 | 155 | LYHSEAFTVNFGDTEEAKK |
| 2186.04 | 2186.0742 | 0.0342 | 16 | 136 | 154 | KLYHSEAFTVNFGDTEEAK |

Project\Project sample_{—}20120802\P12139-2fncbi

| | | | | | | |
|---|---|---|---|---|---|---|
| 2574.3411 | 2574.3052 | −0.0359 | −14 | 102 | 125 | TLNQPDSQLQL |
| | | | | | | TIFLSEGLK |

Along with these three newly discovered proteins for all the products derived from fraction IV, we have found the following existing discovered proteins from AFCC® in which in which good KH healthy cells exist AFCC® protein and their sequence:
KH53
Chain E, The Isomorphous Structures Of Prethrombin2
coagulation factor II (thrombin), isoform CRA_b
Chain B, Crystal Structure Of Human Coagulation Factor Xa
Chain A, Crystal Structure Of Prethrombin-2FRAGMENT-2 Complex
inter-alpha (globulin) inhibitor H2

### FIG. 13F

| Gel Idx/Pos | | 108/E7 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|194373497 | unnamed protein | |
| | | product | |
| | | [Homo sapiens] | |

KH53 (SEQ ID NO. KH 53)
Peptide Information
Project\Project
sample_20120802\P12139-
2fncbi

| | Obsrv. | | | Start | End | |
|---|---|---|---|---|---|---|
| Calc. Mass | Mass | ±da | ±ppm | Seq. | Seq. | Sequence |
| 836.4009 | 836.3804 | −0.0205 | −25 | 67 | 73 | GQQYQGR |
| 836.4009 | 836.3804 | −0.0205 | −25 | 67 | 73 | GQQYQGR |
| 1189.579 | 1189.5811 | 0.0021 | 2 | 449 | 457 | YGFYTHVFR |
| 1189.579 | 1189.5811 | 0.0021 | 2 | 449 | 457 | YGFYTHVFR |
| 1194.6001 | 1194.5938 | −0.0063 | −5 | 203 | 212 | ELLESYIDGR |
| 1464.7805 | 1464.7512 | −0.0293 | −20 | 336 | 349 | ETAASLLQAGYKGR |
| 1489.7223 | 1489.7413 | 0.019 | 13 | 446 | 457 | DGKYGFYTHVFR |
| 1489.7223 | 1489.7413 | 0.019 | 13 | 446 | 457 | DGKYGFYTHVFR |
| 1515.8352 | 1515.8251 | −0.0101 | −7 | 290 | 301 | ISMLEKIYIHPR |
| 1518.8275 | 1518.6852 | −0.1423 | −94 | 265 | 277 | NFTENDLLVRIGK |
| 1526.6251 | 1526.655 | 0.0299 | 20 | 410 | 424 | GDACEGDSGGPFVMK |
| 1542.62 | 1542.7037 | 0.0837 | 54 | 410 | 424 | GDACEGDSGGPFVMK |
| 1561.7281 | 1561.7467 | 0.0186 | 12 | 164 | 176 | TATSEYQTFFNPR |
| 1561.7281 | 1561.7467 | 0.0186 | 12 | 164 | 176 | TATSEYQTFFNPR |
| 1645.8214 | 1645.8168 | −0.0046 | −3 | 234 | 248 | SPQELLCGASLISDR |
| 1682.7262 | 1682.7006 | −0.0256 | −15 | 409 | 424 | RGDACEGDSGGPFVMK |
| 1860.9603 | 1860.9722 | 0.0119 | 6 | 350 | 366 | VTGWGNLKETWTANVGK |
| 1883.8956 | 1883.9106 | 0.015 | 8 | 177 | 193 | TFGSGEADCGLRPLFEK |
| 1883.8956 | 1883.9106 | 0.015 | 8 | 177 | 193 | TFGSGEADCGLRPLFEK |
| 1963.908 | 1963.8922 | −0.0158 | −8 | 97 | 112 | HQDFNSAVQLVENFCR |
| 1995.0481 | 1995.0499 | 0.0018 | 1 | 74 | 92 | LAVTTHGLPCLAWASAQ |
| | | | | | | AK |
| 1995.0481 | 1995.0499 | 0.0018 | 1 | 74 | 92 | LAVTTHGLPCLAWASAQ |
| | | | | | | AK |
| 2001.9158 | 2001.9205 | 0.0047 | 2 | 393 | 409 | ITDNMFCAGYKPDEGKR |
| 2011.9906 | 2012.0179 | 0.0273 | 14 | 177 | 194 | TFGSGEADCGLRPLFEKK |
| 2011.9906 | 2012.0179 | 0.0273 | 14 | 177 | 194 | TFGSGEADCGLRPLFEKK |
| 2017.9106 | 2018.0586 | 0.148 | 73 | 393 | 409 | ITDNMFCAGYKPDEGKR |
| 2232.2534 | 2232.2668 | 0.0134 | 6 | 367 | 386 | GQPSVLQVVNLPIVERPV |
| | | | | | | CK |
| 2232.2534 | 2232.2668 | 0.0134 | 6 | 367 | 386 | GQPSVLQVVNLPIVERPV |
| | | | | | | CK |
| 2281.0991 | 2281.1819 | 0.0828 | 36 | 213 | 232 | IVEGSDAEIGMSPWQVM |
| | | | | | | LFR |
| 2888.3591 | 2888.3918 | 0.0327 | 11 | 48 | 73 | SEGSSVNLSPPLEQCVP |
| | | | | | | DRGQQYQGR |
| 2888.3591 | 2888.3918 | 0.0327 | 11 | 48 | 73 | SEGSSVNLSPPLEQCVP |
| | | | | | | DRGQQYQGR |

| Gel Idx/Pos | | 109/E8 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|809450 | Chain E, The Isomorphous Structures Of Prethrombin2, | |
| | | Hirugen-And Ppack-Thrombin: Changes Accompanying | |
| | | Activation And Exosite Binding To | |
| | | Thrombin | |

Peptide Information

| Calc. | Obsrv. | | | Start | End | |
|---|---|---|---|---|---|---|
| Mass | Mass | ±da | ±ppm | Seq. | Seq. | Sequence |
| 874.4781 | 874.436 | −0.0421 | −48 | 174 | 181 | VTGWGNLK |
| 977.4897 | 977.473 | −0.0167 | −17 | 19 | 26 | SLEDKTER |
| 1189.579 | 1189.584 | 0.005 | 4 | 273 | 281 | YGFYTHVFR |
| 1189.579 | 1189.584 | 0.005 | 4 | 273 | 281 | YGFYTHVFR |
| 1194.6001 | 1194.6135 | 0.0134 | 11 | 27 | 36 | ELLESYIDGR |
| 1265.6022 | 1265.6649 | 0.0627 | 50 | 126 | 134 | YNWRENLDR |
| 1464.7805 | 1464.7592 | −0.0213 | −15 | 160 | 173 | ETAASLLQAGYKGR |
| 1489.7223 | 1489.7659 | 0.0436 | 29 | 270 | 281 | DGKYGFYTHVFR |
| 1489.7223 | 1489.7659 | 0.0436 | 29 | 270 | 281 | DGKYGFYTHVFR |
| 1515.8352 | 1515.8552 | 0.02 | 13 | 114 | 125 | ISMLEKIYIHPR |
| 1526.6251 | 1526.6322 | 0.0071 | 5 | 234 | 248 | GDACEGDSGGPFVMK |
| 1542.62 | 1542.5978 | −0.0222 | −14 | 234 | 248 | GDACEGDSGGPFVMK |
| 1682.7262 | 1682.7395 | 0.0133 | 8 | 233 | 248 | RGDACEGDSGGPFVMK |
| 1698.7211 | 1698.7263 | 0.0052 | 3 | 233 | 248 | RGDACEGDSGGPFVMK |
| 1859.7841 | 1859.9163 | 0.1322 | 71 | 255 | 269 | WYQMGIVSWGEGCDR |
| 1860.9603 | 1860.9381 | −0.0222 | −12 | 174 | 190 | VTGWGNLKETWTANVGK |
| 1860.9603 | 1860.9381 | −0.0222 | −12 | 174 | 190 | VTGWGNLKETWTANVGK |
| 1883.8956 | 1883.9144 | 0.0188 | 10 | 1 | 17 | TFGSGEADCGLRPLFEK |
| 1883.8956 | 1883.9144 | 0.0188 | 10 | 1 | 17 | TFGSGEADCGLRPLFEK |
| 2001.9158 | 2001.8892 | −0.0266 | −13 | 217 | 233 | ITDNMFCAGYKPDEGKR |
| 2001.9158 | 2001.8892 | −0.0266 | −13 | 217 | 233 | ITDNMFCAGYKPDEGKR |
| 2011.9906 | 2011.9617 | −0.0289 | −14 | 1 | 18 | TFGSGEADCGLRPLFEKK |
| 2011.9906 | 2011.9617 | −0.0289 | −14 | 1 | 18 | TFGSGEADCGLRPLFEKK |
| 2017.9106 | 2017.9246 | 0.014 | 7 | 217 | 233 | ITDNMFCAGYKPDEGKR |

Project\Project sample_{—}20120802T12139-2fncbi

| | | | | | | |
|---|---|---|---|---|---|---|
| 2114.9998 | 2114.981 | −0.0188 | −9 | 215 | 232 | IRITDNMFCAGYKPDEGK |
| 2232.2534 | 2232.2441 | −0.0093 | −4 | 191 | 210 | GQPSVLQVVNLPIVERPVCK |
| 2232.2534 | 2232.2441 | −0.0093 | −4 | 191 | 210 | GQPSVLQVVNLPIVERPVCK |
| 2255.2004 | 2255.1882 | −0.0122 | −5 | 141 | 159 | LKKPVAFSDYIHPVCLPDR |
| 2265.1042 | 2265.0938 | −0.0104 | −5 | 37 | 56 | IVEGSDAEIGMSPWQVMLFR |
| 2281.0991 | 2281.1055 | 0.0064 | 3 | 37 | 56 | IVEGSDAEIGMSPWQVMLFR |
| 2297.0942 | 2297.1052 | 0.011 | 5 | 37 | 56 | IVEGSDAEIGMSPWQVMLFR |
| 2691.4612 | 2691.4736 | 0.0124 | 5 | 191 | 214 | GQPSVLQVVNLPIVERPVCKDSTR |

| | | | |
|---|---|---|---|
| Gel Idx/Pos | | 110/E9 | Instr./Gel Origin |
| Plate [#] Name | | [1] Sample Project | Instrument |
| | | 20120802 | Sample Name |
| Rank | Accession No. | Protein Name | |

| | |
|---|---|
| gi\|119588384 | coagulation factor II |
| | (thrombin), isoform |
| | CRA_b |
| | [Homo sapiens] |

Peptide Information

| | | | | | | |
|---|---|---|---|---|---|---|
| Calc. Mass | Obsrv. | ±da | ±ppm | Start | End | Sequence |
| | Mass | | | Seq. | Seq. | |
| Calc. Mass | Obsrv. | ±da | ±ppm | | | Sequence |
| | Mass | | | | | |

Project\Project sample_{—}20120802\P12139-2fncbi

| | | | | | | |
|---|---|---|---|---|---|---|
| 971.425 | 971.3926 | −0.0324 | −33 | 87 | 94 | YTACETAR |
| 971.425 | 971.3926 | −0.0324 | −33 | 87 | 94 | YTACETAR |
| 1148.5518 | 1148.5323 | −0.0195 | −17 | 125 | 133 | SGIECQLWR |
| 1148.5518 | 1148.5323 | −0.0195 | −17 | 125 | 133 | SGIECQLWR |
| 1172.5074 | 1172.4919 | −0.0155 | −13 | 544 | 553 | ITDNMFCAGK |
| 1194.6001 | 1194.5298 | −0.0703 | −59 | 354 | 363 | ELLESYIDGR |
| 1220.6271 | 1220.5682 | −0.0589 | −48 | 416 | 425 | NFTENDLLVR |
| 1968.8903 | 1968.8732 | −0.0171 | −9 | 100 | 117 | LAACLEGNCAEGLGTNYR |
| 2154.9397 | 2154.9436 | 0.0039 | 2 | 159 | 177 | NPDSSTTGPWCYTTDPTVR |
| 2212.0122 | 2212.0112 | −0.001 | 0 | 98 | 117 | DKLAACLEGNCAEGLGTNYR |
| 2212.0122 | 2212.0112 | −0.001 | 0 | 98 | 117 | DKLAACLEGNCAEGLGTNYR |
| 2276.0469 | 2276.0325 | −0.0144 | −6 | 179 | 198 | QECSIPVCGQDQVTVAMTPR |
| 2292.0417 | 2292.0347 | −0.007 | −3 | 179 | 198 | QECSIPVCGQDQVTVAMTPR |
| 2432.1479 | 2432.1167 | −0.0312 | −13 | 178 | 198 | RQECSIPVCGQDQVTVAMTPR |
| 2432.1479 | 2432.1167 | −0.0312 | −13 | 178 | 198 | RQECSIPVCGQDQVTVAMTPR |
| 2448.1428 | 2448.137 | −0.0058 | −2 | 178 | 198 | RQECSIPVCGQDQVTVAMTPR |
| 2448.1428 | 2448.137 | −0.0058 | −2 | 178 | 198 | RQECSIPVCGQDQVTVAMTPR |

| Gel Idx/Pos | | 111/E10 | Instr./Gel Origin |
|---|---|---|---|
| Plate [#] | | [1] Sample Project | Instrument |
| Name | | 20120802 | Sample Name |
| Rank | Accession No. | Protein Name | |
| | gi\|10835687 | Chain B, Crystal | |
| | | Structure Of Human | |
| | | Coagulation | |
| | | Factor Xa Cornplexed | |
| | | With Rpr128515 | |

Peptide Information

| | Obsrv. | | | Start | End | |
|---|---|---|---|---|---|---|
| Calc. Mass | Mass | ±da | ±ppm | Seq. | Seq. | Sequence |
| 938.4366 | 938.3956 | −0.041 | −44 | 32 | 38 | TNEFWNK |
| 939.4352 | 939.3825 | −0.0527 | −56 | 75 | 81 | NCELFTR |
| 939.4352 | 939.3825 | −0.0527 | −56 | 75 | 81 | NCELFTR |
| 968.4207 | 968.4991 | 0.0784 | 81 | 24 | 31 | EVFEDSDK |
| 1067.5303 | 1067.4808 | −0.0495 | −46 | 75 | 82 | NCELFTRK |
| 1067.5303 | 1067.4808 | −0.0495 | −46 | 75 | 82 | NCELFTRK |
| 1320.6075 | 1320.5415 | −0.066 | −50 | 118 | 129 | ACIPTGPYPCGK |
| 1320.6075 | 1320.5415 | −0.066 | −50 | 118 | 129 | ACIPTGPYPCGK |
| 1723.6647 | 1723.5886 | −0.0761 | −44 | 41 | 55 | DGDQCETSPCQNQGK |
| 1723.6647 | 1723.5886 | −0.0761 | −44 | 41 | 55 | DGDQCETSPCQNQGK |
| 2014.823 | 2014.7344 | −0.0886 | −44 | 39 | 55 | YKDGDQCETSPCQNQGK |
| 2014.823 | 2014.7344 | −0.0886 | −44 | 39 | 55 | YKDGDQCETSPCQNQGK |
| 2223.9355 | 2223.8198 | −0.1157 | −52 | 56 | 74 | CKDGLGEYTCTCLEGFE |
| | | | | | | GK |
| 2240.0476 | 2239.9363 | −0.1113 | −50 | 109 | 129 | GYTLADNGKACIPTGPYP |
| | | | | | | CGK |
| 2240.0476 | 2239.9363 | −0.1113 | −50 | 109 | 129 | GYTLADNGKACIPTGPYP |
| | | | | | | CGK |

| Gel Idx/Pos | | 112/E11 | Instr./Gel Origin |
|---|---|---|---|
| Plate [#] Name | | [1] Sample Project | Instrument |
| | | 20120802 | Sample Name |
| Rank | Accession No. | Protein Name | |
| | gi\|307776408 | Chain A, Crystal | |
| | | Structure Of | |
| | | Prethombin- | |
| | | 2FRAGMENT-2 | |
| | | Complex | |

Peptide Information

| | Obsrv. | | | Start | End | |
|---|---|---|---|---|---|---|
| Calc. Mass | Mass | ±da | ±ppm | Seq. | Seq. | Sequence |
| 836.4009 | 836.3383 | −0.0626 | −75 | 20 | 26 | GQQYQGR |
| 836.4009 | 836.3383 | −0.0626 | −75 | 20 | 26 | GQQYQGR |
| 1963.908 | 1963.8728 | −0.0352 | −18 | 50 | 65 | HQDFNSAVQLVENFCR |
| 1995.0481 | 1994.9705 | −0.0776 | −39 | 27 | 45 | LAVTTHGLPCLAWASAQ |
| | | | | | | AK |
| 1995.0481 | 1994.9705 | −0.0776 | −39 | 27 | 45 | LAVTTHGLPCLAWASAQ |
| | | | | | | AK |
| 2070.9761 | 2070.8914 | −0.0847 | −41 | 1 | 19 | SEGSSVNLSPPLEQCVP |
| | | | | | | DR |
| 2888.3591 | 2888.2969 | −0.0622 | −22 | 1 | 26 | SEGSSVNLSPPLEQCVP |
| | | | | | | DRGQQYQGR |
| 2888.3591 | 2888.2969 | −0.0622 | −22 | 1 | 26 | SEGSSVNLSPPLEQCVP |
| | | | | | | DRGQQYQGR |

| Gel Idx/Pos | | 113/E12 | Instr./Gel Origin |
|---|---|---|---|
| Plate [#] | | [1] Sample Project | Instrument |
| Name | | 20120802 | Sample Name |
| Rank | Accession No. | Protein Name | |
| | gi\|55958061 | inter-alpha (globulin) | |
| | | inhibitor H2 | |
| | | [Homo sapiens] | |

Peptide Information

| Calc. | Obsrv. | | | Start | End | |
|---|---|---|---|---|---|---|
| Mass | Mass | ±da | ±ppm | Seq. | Seq | Sequence |
| 846.4832 | 846.4487 | −0.0345 | −41 | 173 | 179 | IYLQPGR |
| 891.4894 | 891.4301 | −0.0593 | −67 | 52 | 59 | VQSTITSR |

Project\Project sample_{—}20120802\P12139-2fncbi

| | | | | | | |
|---|---|---|---|---|---|---|
| 891.4894 | 891.4301 | −0.0593 | −67 | 52 | 59 | QSTITSR |
| 1010.5009 | 1010.4343 | −0.0666 | −66 | 60 | 68 | MATTMIQSK |
| 1010.5009 | 1010.4343 | −0.0666 | −66 | 60 | 68 | MATTMIQSK |
| 1026.4958 | 1026.4567 | −0.0391 | −38 | 60 | 68 | MATTMIQSK |
| 1042.4907 | 1042.4937 | 0.003 | 3 | 60 | 68 | MATTMIQSK |
| 1158.6993 | 1158.6176 | −0.0817 | −71 | 173 | 182 | IYLQPGRLAK |
| 2122.1292 | 2122.0242 | −0.105 | −49 | 69 | 87 | VVNNSPQPQNVVFDVQIPK |
| 2122.1292 | 2122.0242 | −0.105 | −49 | 69 | 87 | VVNNSPQPQNVVFDVQIPK |
| 2178.0859 | 2177.9634 | −0.1225 | −56 | 132 | 151 | SSALDMENFRTEVNVLPGAK |

In addition to these a lot more new found KH proteins and existing proteins whose cells are good healthy KH cells are being discovered from two series (32) process from the manufacturing of AFOD® from fraction IV and AFCC® from fraction III (under different patent application).

1. KH1 (new found protein from Cryoprecipitate)—Sequence: SEQ ID. NO. KH1.

2. KH2 (new found protein from Cryoprecipitate)—Sequence SEQ ID. NO. KH2.

3. KH3 (new found protein from Fraction III)—Sequence: SEQ ID. No. KH3.

4. KH4 (new found protein from fraction III)—Sequence: SEQ ID. No. KH4.

5. KH5 (new found protein from fraction III)—Sequence SEQ ID. NO. KH5.

6. KH6 new found protein from fraction III)—Sequence SEQ ID. NO. KH6.

7. KH7 (new found protein from fraction III)—Sequence SEQ ID. NO. KH7.

8. KH8 (new found protein from fraction III)—Sequence SEQ ID. NO. KH8.

9. KH9 (new found protein from fraction III)—Sequence SEQ ID. NO. KH9.

10. KH10 (new found protein from fraction III)—Sequence SEQ ID. NO. KH10.

11. KH11 (new found protein from Prothrombin Complex Concentrate)—Sequence SEQ ID. NO. KH11.

12. KH12 (new found protein from Prothrombin Complex Concentrate)—Sequence SEQ ID. NO. KH12.

13. KH13 (new found protein from Prothrombin Complex Concentrate)—Sequence SEQ ID. NO. KH13.

14. KH14 (new found protein from Prothrombin Complex Concentrate)—Sequence SEQ ID. NO. KH14.

15. KH15 (new found protein from Prothrombin Complex Concentrate)—Sequence SEQ ID. NO. KH15.

16. KH16 (new found protein from Prothrombin Complex Concentrate)—Sequence SEQ ID. NO. KH16.

17. KH17 (new found protein from Prothrombin Complex Concentrate)—Sequence SEQ ID. NO. KH17.

18. KH18 (new found protein from Prothrombin Complex Concentrate)—Sequence SEQ ID. NO. KH18.

19. KH19 (new found protein from AFCC)—Sequence SEQ ID. NO. KH19.

20. KH20 (new found protein from AFCC)—Sequence SEQ ID. NO. KH20.

21. KH 21 (new found protein from fraction IV)—Sequence SEQ ID. NO. KH21.

22. KH 22 (new found protein from fraction IV)—Sequence SEQ ID. NO. KH22.

23. KH 23 (new found protein from fraction IV)—Sequence SEQ ID. NO. KH23.

24. KH 24 (new found protein from AFOD)—Sequence SEQ ID. NO. KH24

25. KH 25 (new found protein from AFOD)—Sequence SEQ ID. NO. KH25.

26. KH 26 (new found protein from AFOD)—Sequence SEQ ID. NO. KH26.

27. KH 27 (new found protein from AFOD)—Sequence SEQ ID NO. KH27.

28. KH28 (New Found protein in HemoRAAS®)—Sequence SEQ ID NO. KH28.

29. KH29 (New Found protein in HemoRAAS®)—Sequence SEQ ID NO. KH29.

30. KH30 (New Found protein in FibroRAAS®)—Sequence SEQ ID NO. KH30.

31. KH31 (New Found protein in FibroRAAS®)—Sequence: SEQ ID NO. KH31.

32. KH32 (New Found protein in FibroRAAS®)—Sequence: SEQ ID NO. KH32.

33. KH33 (New Found protein in GammaRAAS®) Sequence: SEQ ID NO. KH33.

34. KH34 (New Found protein in GammaRAAS®)—Sequence: SEQ ID NO. KH34.

35. KH35 (New Found protein in GammaRAAS®)—Sequence: SEQ ID NO. KH35.

36. KH36 (New Found protein in GammaRAAS®)—Sequence: SEQ ID NO. KH36.

37. KH37 (New Found protein in GammaRAAS®)—Sequence: SEQ ID NO. KH 37.

38. KH38 (New Found protein in AFCC®)—Sequence: SEQ ID NO. KH38.

39. KH39 (New Found protein in AFCC®)—Sequence: SEQ ID NO. KH39.

40. KH40 (New Found protein in AFCC®)—Sequence: SEQ ID NO. KH40.

41. KH41 (New Found protein in Fraction 3-2)—Sequence: SEQ ID NO. KH41.

42. KH42 (New Found protein in Fraction 3)—Sequence: SEQ ID NO. KH42.

43. KH43 (New Found protein in Fraction 3)—Sequence: SEQ ID NO. KH43.

44. KH44 (New Found protein in FibingluRAAS®Thrombin)—Sequence: SEQ ID NO. KH44.

45. KH45 (New Found protein in FibingluRAAS®Thrombin)—Sequence: SEQ ID NO. KH45.

46. KH46 (New Found protein in FibingluRAAS®Thrombin)—Sequence: SEQ ID NO. KH46.

47. KH47 (New Found protein in FibingluRAAS®Thrombin)—Sequence: SEQ ID NO. KH47.

48. KH48 (New Found protein in AFOD®)—Sequence: SEQ ID NO. KH48.

49. KH49 (New Found protein in AFOD®)—Sequence: SEQ ID NO. KH49.

50. KH50 (New Found protein in AFOD®)—Sequence: SEQ ID NO. KH50.

51. KH51 (New Found protein in AlbuRAAS®)—Sequence: SEQ ID NO. KH51.

52. KH52 (New Found protein in FibingluRAAS® High Concentrate Fibrinogen)—Sequence: SEQ ID NO. KH52.

53. KH53 protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

54. KH54 protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

55. KH55 protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

56. Chain A, The Intact And Cleaved Human Antithrombin III protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

57. Beta-2-glycoprotein I protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

58. hCG1646813 protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

59. Creatine kinase, muscle protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

60. Similar to ring finger protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

61. Chain A, Alpha-1-Antitrypsin protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

62. Chain A, Tertiary Structures Of Three Amyloidogenic Transthyretin Variants And Implications For Amyloid Fibril Formation protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

63. Chain A, Apo-Human Serum Transferrin (Glycosylated) protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

64. hemopexin, isoform CRA_c protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

65. Alpha-1-Antitrypsin protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

66. Chain A, 2.0 Angstrom Structure Of IntactAlpha-1-Antitrypsin protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

67. Chain E, The Isomorphous Structures Of Prethrombin2 protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

68. coagulation factor II (thrombin), isoform CRA_b protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

69. Chain B, Crystal Structure Of Human Coagulation Factor Xa protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

70. Chain A, Crystal Structure Of Prethrombin-2FRAGMENT-2 Complex protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

71. inter-alpha (globulin) inhibitor H2 protein whose KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

72. KH 53 new found protein and its sequence: SEQ ID NO. KH53.

73. KH54 new found protein and its sequence: SEQ ID NO. KH54.

74. KH55 new found protein and its sequence: SEQ ID NO. KH55.
